(19) United States
(12) Patent Application Publication
(10) Pub. No.: US 2013/0171103 A1
(43) Pub. Date: Jul. 4, 2013

(54) METHODS FOR TREATING VIRAL CONDITIONS

(71) Applicants: Thomas Davis, South Orange, NJ (US); Jason D. Graci, Scranton, PA (US); Zhengxian Gu, Princeton, NJ (US); Christopher Trotta, Somerset, NJ (US)

(72) Inventors: Thomas Davis, South Orange, NJ (US); Jason D. Graci, Scranton, PA (US); Zhengxian Gu, Princeton, NJ (US); Christopher Trotta, Somerset, NJ (US)

(73) Assignee: PTC Therapeutics, Inc., South Plainfield, NJ (US)

(21) Appl. No.: 13/699,849

(22) PCT Filed: May 26, 2011
(86) PCT No.: PCT/US11/38067

§ 371 Date: Mar. 22, 2013

Related U.S. Application Data
(60) Provisional application No. 61/349,186, filed on May 27, 2010.

(51) Int. Cl. C07D 471/04; A61K 45/06; A61K 31/4545; A61K 38/21; A61K 31/7056; A61K 39/29; A61K 39/42; A61K 31/7088; A61K 31/437; A61K 31/5377

(52) U.S. Cl. CPC C07D 471/04; A61K 31/437; A61K 45/06; A61K 31/4545; A61K 31/5377; A61K 31/7056; A61K 39/29; A61K 39/42; A61K 31/7088; A61K 38/21
USPC 424/85.4; 514/292; 514/232.8; 514/43; 424/228.1; 514/3.7; 424/147.1; 514/44 A

(57) ABSTRACT

Compounds that selectively inhibit viral replication or the production of viral RNA or DNA, viral protein or virus induced cytopathic effects and compositions comprising such Compounds are described. Also described are methods of inhibiting viral replication or the production of viral RNA or DNA, viral protein or virus induced cytopathic effects using such Compounds and methods for treating viral infections involving the administration of such Compounds. The Compounds may be administered as a single agent therapy or in combination with one or more additional therapies to a human in need of such treatments.

### 1. CROSS-REFERENCE TO RELATED APPLICATIONS

This application claims the benefit of and priority to U.S. Provisional Application No. 61/349,186, filed May 27, 2010, which is incorporated herein by reference herein in its entirety.

### 2. INTRODUCTION

Compounds that selectively inhibit viral replication or the production of viral RNA or DNA, viral protein or virus induced cytopathic effects and compositions comprising such Compounds are described. Further described are methods of inhibiting viral replication or the production of viral RNA or DNA, viral protein or virus induced cytopathic effects using such Compounds and methods for treating viral infections involving the administration of such Compounds. The Compounds may be administered as a single agent therapy or in combination with one or more additional therapies to a human in need of such treatments.

### 3. BACKGROUND

3.1 Viral Conditions

As obligate intracellular parasites, viruses are intimately dependent upon the biological functions of their hosts. Small molecules that affect the host cell biological processes involved in viral replication or the production of viral RNA or DNA, viral protein or virus induced cytopathic effects may therefore inhibit a wide variety of viruses requiring these functions for essential events in the viral life cycle and therefore can be used for treatment of virus infection. Notably, molecules directly affecting host functions that are essential for viral replication or the production of viral RNA or DNA, viral protein or virus induced cytopathic effects should provide a high barrier to the emergence of resistant strains relative to classical antivirals that directly target viral enzymes.

An estimated 170 million people worldwide are reported to be infected with hepatitis C virus (HCV), of which at least 6 known genotypes are the causative agent of hepatitis C infection. Up to 80 percent of HCV infections lead to chronic liver infection, which in turn may result in severe liver diseases, including liver fibrosis, cirrhosis, and hepatocellular carcinoma (see Saito I, et al., Hepatitis C virus infection is associated with the development of hepatocellular carcinoma, Proc Natl Acad Sci USA, 1990, 87:6547-6549). References have described small molecule β-carboline compounds with antiviral activity against viruses such as human papillomavirus (HPV)(J F Miller et al, Bioorganic & Medicinal Chemistry Letters, 2010, 20(1):256-259), poliovirus (PV) and herpes simplex virus (HSV) (ASN Formagio et al, European Journal of Medicinal Chemistry, 2009, 44(11):4695-4701). International Patent publications WO2006/015035 and WO2007/002051 describe β-carboline compounds with antiviral activity against human papillomavirus infection (HPV) and a flavivirus infection, including dengue virus, yellow fever virus, West Nile virus and HCV infection. Accordingly, new small molecule therapies for treating patients with viral conditions, particularly dengue virus and HCV, are needed.

### 4. SUMMARY

Encompassed herein are compounds having the formula set forth herein and compositions comprising such Compounds. The Compounds can demonstrate one or more of the following activities: (a) prolongation of the G1/S phase of the cell cycle; and/or (b) inhibition of a viral infection.

Methods for treating or preventing viral infections are described, involving the administration of a Compound to a human subject in need of such treatment. Preferably, the Compound used in the therapeutic method demonstrates one or more of the following activities as determined in cell culture and/or animal model systems, such as those described herein: (a) prolongation of the G1/S phase of the cell cycle; and/or (b) inhibition of viral infection. The Compound can be administered as a single agent therapy to a human in need of such treatment. Alternatively, the Compound can be administered in combination with one or more additional therapies to a human in need of such treatment. Such therapies may include the use of antiviral agents.

The therapies described herein should be effective because they are aimed at interfering with basic mechanisms required for the manifestation of disease (i.e., the biological processes involved in viral replication or the production of viral RNA or DNA, viral protein or virus induced cytopathic effects). While not bound by any theory, the therapies described are based, in part, on the pharmacodynamic activities of the Compounds as measured in cell culture and in animal models; in particular, these include: (a) prolongation of the G1/S phase of the cell cycle of aberrantly proliferating cells and/or (b) inhibition of viral replication or the production of viral RNA or DNA, viral protein or virus induced cytopathic effects.

These pharmacologic activities contribute to limiting viral infection in several ways. For example, prolongation of the cell cycle may contribute to the induction of apoptotic death of the infected cells, and/or allow for increased efficacy when the Compound is used in combination with a therapy or therapies (e.g., antiviral agents) that interfere with nucleic acid synthesis during the cell cycle (e.g., the G1/S phase). Because viral replication is directly dependent on host cells, a Compound that interferes with cellular molecular processes that participate in viral replication may inhibit one or more events of the viral life cycle and thus be used for treatment of a viral infection. Finally, a Compound that interferes with viral replication or the production of viral RNA or DNA, viral protein or virus induced cytopathic effects may inhibit relapse of one or more symptoms associated with recurrence of a viral infection.

Thus, in specific embodiments, the methods for treating a viral infection can result in inhibition or reduction of the viral infection, thus reducing viral DNA, RNA or viral proteins in biological specimens of an afflicted subject; inhibition of viral latency in the subject; stabilization or reduction of the viral titer in the subject; stabilization or reduction of organ pathology and/or organ failure in the subject; reduction of the concentrations of viral DNA, RNA or viral proteins in biological specimens (e.g., plasma, serum, urine, or infected tissues); and/or a delayed or prolonged late G1/S phase of the cell cycle (i.e., the period between the late resting or pre-DNA synthesis phase, and the early DNA synthesis phase) in infected cells of the subject. Without being bound by any particular theory, the methods of treating a viral infection can result in interference with viral replication in infected cells in an afflicted subject and prevent or reduce the ability of the virus to appropriate the host apparatus and molecular processes in the subject.

Existing antiviral therapies are a combination of interferon and ribavirin, leading to variable outcomes among the six major HCV genotypes. However, only about one-half of all treated patients respond to this combination therapy. Since the Compounds used in the therapeutic methods described herein are small molecules that selectively inhibit viral replication or the production of viral RNA or DNA, viral protein or virus induced cytopathic effects, side effects that are unacceptable for standard antiviral treatment may be reduced.

The efficacy of the therapeutic intervention is supported by the data presented herein, demonstrating that the Compounds delay the cell cycle by prolonging the G1/S phase; and that the Compounds inhibit viral replication or the production of viral RNA or DNA, viral protein or virus induced cytopathic effects by interfering with biological processes involved in viral replication or the production of viral RNA or DNA, viral protein or virus induced cytopathic effects.

4.1 Definitions

As used herein, the term “effective amount” in the context of administering a Compound to a subject with a viral infection refers to the amount of a Compound that results in a beneficial or therapeutic effect. In specific embodiments, an “effective amount” of a Compound refers to an amount of a Compound which is sufficient to achieve at least one, two, three, four or more of the following effects: (i) the reduction or amelioration of the severity of one or more symptoms associated with viral infection; (ii) the reduction in the duration of one or more symptoms associated with viral infection; (iii) the prevention in the recurrence of a viral infection or one or more symptoms associated with viral infection; (iv) the regression of viral infection and/or one or more symptoms associated therewith; (v) the inhibition of the progression of viral infection and/or one or more symptoms associated therewith; (vi) the enhancement of and/or improvement of the therapeutic effect of another antiviral therapy; (vii) a reduction in a viral titer; (viii) a reduction in the progression of viral infection; (ix) a reduction in viral sequestration and/or latency; (x) a decrease in viral proteins in the cells of a subject having a viral infection; (xi) an increase in relapse free infection; (xii) an increase in the number of patients in remission of viral infection; (xiii) a decrease in hospitalization rate associated with viral infection; (xiv) a decrease in organ transplant rate associated with viral infection; (xv) the prevention of the development or onset of one or more symptoms associated with viral infection; (xvi) an increase in the length of remission of viral infection in patients; (xvii) the reduction in the number of one or more symptoms associated with viral infection; (xviii) an increase in symptom-free survival of patients having a viral infection; (xix) a decrease in the concentration of circulating viral RNA or DNA or viral protein in the plasma of a subject having a viral infection; (xx) a decrease in viral replication in the cells of a subject having a viral infection; (xxi) a decrease in the concentration of viral RNA or DNA, viral protein or the production of virus induced cytopathic effects in a biological specimen (e.g., the plasma, serum, urine or tissue of a subject having a viral infection; (xxii) the inhibition or reduction in viral re-infection following organ transplant; (xxiii) the inhibition or reduction in the occurrence of viral infection following a period of latency; (xxiv) improvement in organ function, e.g., liver cirrhosis; (xxv) a decrease in organ function pathology, e.g., liver failure; (xxvi) the inhibition or reduction in production of viral RNA or DNA, viral protein or virus induced cytopathic effects; (xxvii) the stabilization or reduction of viral replication in the cells of a subject; (xxviii) the reduction of the concentration of viral RNA or DNA or viral protein or other viral mediators (e.g., chemokines, cytokines or interleukins) in biological specimens (e.g., plasma, serum, urine, or any other biofluids or tissue specimens); (xxix) the decrease in production of viral proteins or virus induced cytopathic effects; (xxx) the inhibition or reduction in viral protein translation; (xxxi) the inhibition or reduction in viral RNA or DNA or viral protein synthesis or production of virus induced cytopathic effects; (xxxii) inhibition or prevention of the formation of a viral replication complex in a cell; (xxxiii) inhibition or prevention of the assembly of a viral replication complex in the endoplasmic reticulum (ER); (xxxiv) inhibition or prevention of the assembly and/or release of viral particles from cells; (xxxv) the improvement in quality of life after a viral infection as assessed by methods well known in the art, e.g., questionnaires; (xxxvi) ease in treating, preventing or ameliorating viral infection by oral delivery of a Compound; and/or (xxxvii) an alteration (e.g, a decrease) in a viral marker (e.g., a decrease of viral RNA or DNA, viral protein or virus induced cytopathic effects in a subject having a viral infection).

As used herein, the term “elderly human” refers to a human 65 years or older.

As used herein, the term “human adult” refers to a human that is 18 years or older.

As used herein, the term “middle-aged human” refers to a human between the ages of 30 and 64.

As used herein, the term “human child” refers to a human that is 1 year to 18 years old.

As used herein, the term “human toddler” refers to a human that is 1 year to 3 years old.

As used herein, the term “human infant” refers to a newborn to 1 year old year human.

As used herein, the terms “subject” and “patient” are used interchangeably to refer to an individual being treated for a viral infection in accordance with the methods provided herein.

As used herein, the terms “therapies” and “therapy” can refer to any protocol(s), method(s), compositions, formulations, and/or agent(s) that can be used in the prevention, treatment, management, or amelioration of a condition or disorder or one or more symptoms thereof (e.g., a viral infection or one or more symptoms or one or more conditions associated therewith). In certain embodiments, the terms “therapies” and “therapy” refer to drug therapy such as adjuvant therapy, surgery, biological therapy, supportive therapy, antiviral therapy and/or other therapies useful in treatment, management, prevention, or amelioration of a condition or disorder or one or more symptoms thereof (e.g., a viral infection or one or more symptoms or one or more conditions associated therewith). In certain embodiments, the term “therapy” refers to a therapy other than a Compound or pharmaceutical composition thereof. In specific embodiments, an “additional therapy” and “additional therapies” refer to a therapy other than a treatment using a Compound or pharmaceutical composition thereof. In a specific embodiment, a therapy includes the use of a Compound as an adjuvant therapy. For example, using a Compound in conjunction with a drug therapy such as biological therapy, surgery, supportive therapy, antiviral therapy and/or other therapies useful in treatment, management, prevention, or amelioration of a condition or disorder or one or more symptoms thereof (e.g., a viral infection or one or more symptoms or one or more conditions associated therewith).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the Compounds provided herein include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein, the term “botanical” generally refers to extracts from natural sources including, without limitation, plant extracts and the like.

As used herein, the term “non-specific pharmaceutical” generally refers to a pharmaceutical agent having multiple known uses including, for example, and without limitation, rapamycin for use as an anti-HCV agent, an antifungal agent, anti-cancer agent, immunosuppressive agent and the like.

As used herein, the terms “Compound” or “Compound provided herein” generally refer to a Compound of Table 1, and pharmaceutically acceptable salts, racemates, tautomers and stereoisomers thereof. In one embodiment, the terms refer to a compound of Formula I, II, III, IV, V, VI or VII. In another embodiment, the terms refer to a compound of Formula Ia, IIa, Ma, IVa, Va, VIIa, VIIa, VIIIa, IXa, Xa, XIa, XIIa, XIIIa or XIVa. In a specific embodiment, the terms refer to a compound depicted in Table 1 and pharmaceutically acceptable salts thereof. In one embodiment, the terms refer to a Compound disclosed in WO2005/089764, e.g., Compounds in the table on pages 26-98; WO2006/113703, e.g., Compounds in the table on pages 29-102; WO2008/127715, e.g., Compounds in the table on pages 52-126; WO2008/127714, e.g., Compounds in the table on pages 48-123; and WO 2010/138758, e.g., Compounds in the table on pages 33-93, all of which are herewith incorporated by reference in their entirety. In certain embodiments, the terms “Compound” or “Compound provided herein” refer to a stereoisomer of a Compound. The “Compound” or “Compound provided herein” may comprise one or more asymmetric carbon atoms, i.e. n asymmetric carbon atoms, having either R or S configuration as determined by a person skilled in the art. In one embodiment, the terms refer to a particular enantiomer, such as an R or S enantiomer of a “Compound” or “Compound provided herein”. In one embodiment, the terms refer to an R or S enantiomer of a compound of Formula I, II, III, IV, V, VI or VII. In another embodiment, the terms refer to an R or S enantiomer of a compound of Formula Ia, IIa, IIIa, IVa, Va, VIIa, VIIa, VIIIa, IXa, Xa, XIa, XIIa, XIIIa or XIVa. In a specific embodiment, the terms refer to an R or S enantiomer of a compound depicted in Table 1. It is understood that the terms “Compound” or “Compound provided herein” encompass all possible stereoisomers that may be generated based on all asymmetric carbon atoms. For example, if a Compound has two (n=2) asymmetric carbon atoms, the terms “Compound” or “Compound provided herein” encompass all four, i.e. 2ⁿ=2²=4, stereoisomers (R,S; R,R; S,S; S;R). The “Compound” or “Compound provided herein” may be a substantially pure (e.g., about 90%, about 95%, about 98%, about 99%, or about 99.9% pure) single stereoisomer or a mixture of two or more stereoisomers.

As used herein, the term “about” means a range around a given value wherein the resulting value is substantially the same as the expressly recited value. In one embodiment, “about” means within 25% of a given value or range. For example, the phrase “about 70% by weight” comprises at least all values from 52% to 88% by weight. In another embodiment, the term “about” means within 10% of a given value or range. For example, the phrase “about 70% by weight” comprises at least all values from 63% to 77% by weight. In another embodiment, the term “about” means within 7% of a given value or range. For example, the phrase “about 70% by weight” comprises at least all values from 65% to 75% by weight.

Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein, the term “viral infection” refers to one or more RNA viruses belonging to families Bunyaviridae, Coronaviridae, Filoviridae, Flaviviridae, Paramyxoviridae, Picornaviridae, Orthomyxoviridae or Rhabdoviridae. Other embodiments include one or more viruses belonging to families Hepadnaviridae, Reoviridae or Retroviridae. Another embodiment includes one or more DNA viruses belonging to families Adenoviridae, Herpesviridae, Papillomaviridae or Papovaviridae.

As used herein, the term “viral replication,” in the context of viral infection, refers to production of viral RNA or DNA or production of one or more viral proteins or production of one or more virus induced cytopathic effects from viruses using double-stranded (ds) DNA or RNA and/or single-stranded (ss) RNA and/or partial-double-stranded (ps) DNA or RNA and/or positive (+) strand RNA and/or negative (−) strand RNA. In one embodiment, the term includes viral DNA replication or viral RNA replication or viral RNA transcription and translation, resulting in the expression of one or more viral proteins by infected cells in tissues of a subject. In another embodiment, the term includes viral expression and/or sequestration and/or latency of viral proteins in chronic viral infection. In another embodiment, the term includes the effect of viruses on cellular biological processes to produce viral RNA or DNA or one or more viral proteins or one or more virus induced cytopathic effects. As applicable, expression of one or more viral proteins may result in viral sequestration and/or latency, inflammation, organ failure and/or tumor growth. The inhibition or reduction in production of viral RNA or DNA or one or more viral proteins or one or more virus induced cytopathic effects by a Compound can be assessed in cell culture and/or animal models as described herein.

As used herein, the term “viral replication complex,” in the context of viral infection, refers to a membrane-associated complex composed of viral proteins, replicating RNA and altered cellular membranes where viral RNA is replicated.

As used herein, unless otherwise specified, the term “substituted” means that a Compound is substituted at one or more positions by one or more substituents where allowed by available valences. Examples of radicals that may be used as substituents are known to those skilled in the art, including those of the compounds described herein.

As used herein, unless otherwise specified, the term “one or more” means that a Compound is substituted at one or more positions by that amount of substituents allowed by available valences.

As used herein, unless otherwise specified, the term “C₁ to C₈ alkyl” means a saturated straight chain or branched non-cyclic hydrocarbon radical having from 1 to 8 carbon atoms, in one embodiment 1-6 carbon atoms, and in another embodiment 1-4 carbon atoms. Representative saturated straight chain C₁ to C₈ alkyl include -methyl, -ethyl, -n propyl, -n-butyl, -n-pentyl, -n-hexyl, -n-heptyl and -n-octyl; while saturated branched C₁ to C₈ alkyl include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl and the like. A C₁ to C₈ alkyl group can be unsubstituted or substituted. Unsaturated alkyl radicals include alkenyl radicals and alkynyl radicals, which are discussed below.

As used herein, unless otherwise specified the term “C₂ to C₈ alkenyl” means a straight chain or branched non-cyclic hydrocarbon radical having from 2 to 8 carbon atoms, in one embodiment 2-6 carbon atoms, and in another embodiment 2-4 carbon atoms, and including at least one carbon-carbon double bond. Representative straight chain and branched C₁ to C₈ alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, and the like. The double bond of an alkenyl radical can be unconjugated or conjugated (where allowed by available valences) to another saturated or unsaturated moiety. An alkenyl radical can be unsubstituted or substituted.

As used herein, unless otherwise specified the term “C₂ to C₈ alkynyl” means a straight chain or branched non-cyclic hydrocarbon radical having from 2 to 8 carbon atoms, in one embodiment 2-6 carbon atoms, and in another embodiment 2-4 carbon atoms, and including at least one carbon-carbon triple bond. Representative straight chain and branched C₂ to C₈ alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, and the like. The triple bond of an alkynyl radical can be unconjugated or conjugated to another saturated or unsaturated moiety. An alkynyl radical can be unsubstituted or substituted.

As used herein, unless otherwise specified the term “cyano” means a radical of the formula: —CN.

As used herein, unless otherwise specified the term “guanidino” means a radical of the formula: —NH—C(═NH)—NH₂. A guanidino may be substituted or unsubstituted.

As used herein, unless otherwise specified the term “halogen” means a radical chlorine, fluorine, bromine or iodine atom of the formula: —Cl, —F, —Br or —I.

As used herein, unless otherwise specified the term “hydroxyl” means a radical of the formula: —OH.

As used herein, unless otherwise specified the term “hydroxyl-C₁ to C₈ alkyl” means a radical of the formula: —C₁ to C₈ alkyl-OH.

As used herein, unless otherwise specified the term “imino” means a radical of the formula: ═NH.

As used herein, unless otherwise specified the term “nitro” means a radical of the formula: —NO₂.

As used herein, unless otherwise specified the term “alkyl-sulfonyl” means a radical of the formula: —SO₂—C₁ to C₈ alkyl, wherein C₁ to C₈ alkyl is defined as above, including —SO₂—CH₃, —SO₂—CH₂CH₃, —SO₂—(CH₂)₂CH₃, —SO₂—(CH₂)₃CH₃, —SO₂—(CH₂)₄CH₃, —SO₂—(CH₂)₅CH₃, and the like.

As used herein, unless otherwise specified the term “cycloalkylcarbonyl” means a radical of the formula: —C(O)-cycloalkyl, wherein cycloalkyl is defined as above, including —C(O)-cyclopropyl, —C(O)-cyclobutyl, —C(O)-cyclopentyl, —C(O)-cyclohexyl, —C(O)-cycloheptyl, —C(O)-cyclooctyl, and the like.

As used herein, unless otherwise specified the term “heteroarylcarbonyl” means a radical of the formula: —C(O)-heteroaryl, wherein heteroaryl is defined as above.

As used herein, unless otherwise specified the term “cycloalkylsulfonyl” means a radical of the formula: —SO₂cycloalkyl, wherein cycloalkyl is defined as above, including —SO₂-cyclopropyl, —SO₂-cyclobutyl, —SO₂-cyclopentyl, —SO₂-cyclohexyl, —SO₂-cycloheptyl, —SO₂-cyclooctyl, and the like.

As used herein, unless otherwise specified the term “carboxyl” and “carboxy” mean a radical of the formula: —COOH or —CO₂H.

As used herein, unless otherwise specified the term “C₁ to C₈ alkoxy” means a radical of the formula: —O—C₁ to C₈ alkyl, wherein C₁ to C₈ alkyl is defined above, including —OCH₃, —OCH₂CH₃, —O(CH₂)₂CH₃, —O(CH₂)₃CH₃, —O(CH₂)₄CH₃, —O(CH₂)₅CH₃, and the like.

As used herein, unless otherwise specified the term “alkoxycarbonyl” means a radical of the formula: —C(═O)—O—C₁ to C₈ alkyl, wherein C₁ to C₈ alkyl is defined above, including —C(═O)O—CH₃, —C(═O)O—CH₂CH₃, —C(═O)O—(CH₂)₂CH₃, —C(═O)O—(CH₂)₃CH₃, —C(═O)O—(CH₂)₄CH₃, —C(═O)O—(CH₂)₅CH₃, and the like. In one embodiment, the esters are biohydrolyzable (i.e., the ester is hydrolyzed to a carboxylic acid in vitro or in vivo).

As used herein, unless otherwise specified the term “alkylcarbonyl” means a radical of the formula: —C(═O)—C₁ to C₈ alkyl, wherein C₁ to C₈ alkyl is defined above, including —C(═O)—CH₃, —C(═O)—CH₂CH₃, —C(═O)—(CH₂)₂CH₃, —C(═O)—(CH₂)₃CH₃, —C(═O)—(CH₂)₄CH₃, —C(═O)—(CH₂)₅CH₃, and the like.

As used herein, unless otherwise specified the term “aryl” means an aromatic carbocyclic ring containing from 6 to 14 ring atoms. The ring atoms of a carbocyclic ring are all carbon atoms. Aryl ring structures include one or more ring structures such as mono-, bi-, or tricyclic as well as fused aromatic carbocyclic moieties. Representative aryl rings include phenyl, anthracenyl, fluorenyl, indenyl, azulenyl, phenanthrenyl, naphthyl and the like. An aryl ring can be unsubstituted or substituted.

As used herein, unless otherwise specified the term “heteroaryl” means a carbocyclic aromatic ring, wherein at least one of the carbocyclic ring atoms is replaced with at least one heteroatom, in one embodiment 1 to 3 heteroatoms, independently selected from nitrogen, oxygen, or sulfur. In one embodiment, the heteroaryl ring is a 5 to 12 membered heteroaryl ring, containing from 5 to 12 ring atom members. Heteroaryl ring structures include one or more ring structures such as mono-, bi-, or tricyclic as well as fused aromatic carbocyclic (i.e. benzo-fused) and heterocarbocyclic moities. Representative heteroaryl rings include triazolyl, tetrazolyl, oxadiazolyl, pyridinyl (also referred to as pyridyl), furanyl, benzofuranyl, thienyl (also referred to as thiophenyl), benzothienyl (also referred to as benzothiophenyl), benzoisoxazolyl, benzoisothiazolyl, quinolinyl, isoquinolinyl, pyrrolyl, indolyl, indazolyl, oxazolyl, benzoxazolyl, imidazolyl, benzimidazolyl, thiazolyl, benzothiazolyl, thiadiazolyl, isoxazolyl, pyrazolyl, isothiazolyl, pyridazinyl, pyrimidinyl, pyrazinyl, triazinyl, cinnolinyl, phthalazinyl, quinazolinyl, benzoquinazolinyl, acridinyl, and the like. A heteroaryl ring can be unsubstituted or substituted.

As used herein, unless otherwise specified the term “cycloalkyl” means a monocyclic or polycyclic saturated or partially unsaturated carbocyclic ring. In one embodiment, the cycloalkyl is a C₃ to C₁₄ cycloalkyl, containing from 3 to 14 ring atom members. Cycloalkyl ring structures include one or more ring structures such as mono-, bi-, or tricyclic as well as fused saturated or aromatic carbocyclic moieties such as 5,6,7,8-tetrahydronaphthalenyl, indanyl, fluorenyl, norbornanyl, adamantanyl and the like. Examples of cycloalkyl rings include, but are not limited to, (C₃-C₇)cycloalkyl groups, including cyclopropyl, cyclobutyl, cyclopentyl, cyclohexyl, and cycloheptyl, and saturated or partially unsaturated cyclic and bicyclic ring systems such as cyclopropenyl, cyclobutenyl, cyclopentenyl, cyclohexenyl, indenyl and the like. A cycloalkyl group can be unsubstituted or substituted. Preferably, the cycloalkyl group is a monocyclic ring or bicyclic ring system.

As used herein, unless otherwise specified the term “heterocyclyl” means a monocyclic or polycyclic saturated or partially unsaturated carbocyclic ring, wherein at least one of the carbocyclic ring atoms is replaced with at least one heteroatom, preferably 1 to 3 heteroatoms, independently selected from nitrogen, oxygen, and sulfur. In one embodiment, the heterocyclyl is a 3 to 12 membered heterocyclyl, containing from 3 to 12 ring atom members. Heterocyclyl ring structures include compounds having one or more ring structures such as mono-, bi-, or tricylic compounds. Preferably, heterocyclyl is a monocyclic ring or bicyclic ring system. Representative heterocyclyl rings include, but are not limited to morpholinyl, thiomorpholinyl, pyrrolidinonyl, pyrrolinyl, pyrrolidinyl, 1,3-dioxolanyl, 1,3-dioxanyl, 1,4-dioxolanyl, imidazolinyl, imidazolidinyl, pyrazolinyl, pyrazolidinyl, piperidinyl, piperazinyl, pyranyl, hydantoinyl, valerolactamyl, oxiranyl, oxetanyl, azetidinyl, tetrahydrofuranyl, tetrahydropyranyl, tetrahydropyridinyl, tetrahydroprimidinyl, tetrahydrothienyl (also referred to as tetrahydrothiophenyl), tetrahydrothiopyranyl, benzo[1,3]dioxolyl, benzo[1,4]dioxanyl, 3H-indolyl, indolinyl, quinuclidinyl and the like. A heterocyclyl ring can be unsubstituted or substituted.

As used herein, unless otherwise specified the term “cycloalkyloxy” means a radical of the formula: —O-cycloalkyl, wherein cycloalkyl is defined above, including, but not limited to —O-cyclopropyl, —O-cyclobutyl, —O-cyclopentyl, —O-cyclohexyl, —O-cycloheptyl and the like.

As used herein, unless otherwise specified the term “amino” means a radical of the formula: —NH₂. An amino group can be substituted or unsubstituted.

As used herein, unless otherwise specified the term “alkylamino” means a radical of the formula: —NH(C₁ to C₈ alkyl) (“monoalkylamino”) or —N(C₁ to C₈ alkyl)(C₁ to C₈ alkyl) (“dialkylamino’), wherein C₁ to C₈ alkyl is defined above, including, but not limited to —NHCH₃, —NHCH₂CH₃, —NH(CH₂)₂CH₃, —NH(CH₂)₃CH₃, —NH(CH₂)₄CH₃, —NH(CH₂)₅CH₃, —N(C H₃)₂, —N(CH₂CH₃)₂, —N((CH₂)₂CH₃)₂, —N(CH₃)(CH₂CH₃), and the like.

As used herein, unless otherwise specified the term “acetamino” means a radical of the formula: —NH—C(O)CH₃.

As used herein, unless otherwise specified the term “phosphonic acid” means a radical of the formula: —P(═O)(OH)₂. A phosphonic acid group can be substituted or unsubstituted.

As used herein, unless otherwise specified the term “phosphonate” means a radical of the formula: —P[(═O)(O—C₁ to C₈ alkyl)₂] or —P[(═O)(OH)(O—C₁ to C₈ alkyl)], wherein C₁ to C₈ alkyl is as defined above.

(C₁ to C₈ alkyl), wherein C₁ to C₈ alkyl is as defined above.

As used herein, unless otherwise specified the term “thioether” means a radical of the formula: —S(C₁ to C₈ alkyl), wherein C₁ to C₈ alkyl is defined above, including —S—CH₃, —S—CH₂CH₃, —S—(CH₂)₂CH₃, —S—(CH₂)₃CH₃, —S—(CH₂)₄CH₃, —S—(CH₂)₅CH₃, and the like. A thioether group can be substituted or unsubstituted.

As used herein, unless otherwise specified the term “thiol” means a radical of the formula: —SH. A thiol group can be substituted or unsubstituted.

As used herein, unless otherwise specified the term “sulfinyl” means a radical of the formula: —S(O)2H. A sulfinyl group can be substituted or unsubstituted.

As used herein, unless otherwise specified the term “sulfonyl” means a radical of the formula: —S(O)₂H. A sulfonyl group can be substituted or unsubstituted.

As used herein, unless otherwise specified the term “oxo” means ═O.

As used herein, unless otherwise specified the term “sulfonamide” means a radical of the formula: —S(O)₂—NH₂. A sulfonamide can be substituted or unsubstituted.

As used herein, unless otherwise specified the term “acetate” means a radical of the formula: —O—C(O)—C₁ to C₈ alkyl. An acetate may be substituted or unsubstituted.

As used herein, unless otherwise specified the term “acetyl” means a radical of the formula: —C(O)—CH₃. An acetyl may be substituted or unsubstituted.

As used herein, unless otherwise specified the term “thiazoleamino” means a radical of the formula: —NH-thiazole. A thiazole amino may be substituted or unsubstituted.

As used herein, unless otherwise specified the term “trimethylsilyl-alkyl-sulfonyl” means a radical of the formula: —SO₂—C₁ to C₈ alkyl-Si(CH₃)₃, wherein C₁ to C₈ alkyl and alkyl-sulfonyl are as defined above, including —SO₂—CH₂—Si(CH₃)₃, —SO₂—(CH₂)₂—Si(CH₃)₃, —SO₂—(CH₂)₃—Si(CH₃)₃, —SO₂—(CH₂)₄—Si(CH₃)₃, —SO₂—(CH₂)₅—Si(CH₃)₃, —SO₂—(CH₂)₆—Si(CH₃)₃, and the like.

As used herein, unless otherwise specified the term “phenyloxy” means a radical of the formula: —O-phenyl. A phenyloxy radical can be unsubstituted or substituted.

As used herein, unless otherwise specified the term “haloalkyl” means a C₁ to C₈ alkyl, as described above, substituted by one or more halogens, as described above.

Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 5. BRIEF DESCRIPTION OF THE DRAWINGS

FIG. 1A-G. Cell Cycle Effects in HT1080 Cells by Compound #10 Concentration. Histograms depicting relative DNA content in HT1080 cells under normoxic conditions after treatment with varying concentrations of Compound #10 compared to vehicle. FIG. 1A. Histogram showing the effect of treatment with vehicle. FIG. 1B-G. Histograms showing the effect of treatment with Compound #10 at 0.3 nm, 1 nm, 3 nm, 10 nm, 30 nm and 100 nm, respectively. The acronyms have the following definitions: G₁=gap 1 phase (resting or pre-DNA synthesis phase—2 chromosomes present); G₂=gap 2 phase (gap between DNA synthesis and mitosis—4 chromosomes present); S=synthesis phase (DNA synthesis ongoing); and, PI=propidium iodide.

FIG. 2A-F. Cell Cycle Effects in HT1080 Cells by Time from Discontinuation of Compound #10. Histograms depicting relative DNA content in HT1080 cells under normoxic conditions after discontinuation of treatment with Compound #10 compared to vehicle. FIG. 2A. Histogram showing the effect of treatment with vehicle. FIGS. 2B-F. Histograms showing the effect of discontinuation of treatment with Compound #10 at 0 hours, 2 hours, 5 hours, 8 hours and 26 hours, respectively. The acronyms have the following definitions: G₁=gap 1 phase (resting or pre-DNA synthesis phase—2 chromosomes present); G₂=gap 2 phase (gap between DNA synthesis and mitosis—4 chromosomes present); S=synthesis phase (DNA synthesis ongoing); and, PI=propidium iodide.

FIG. 3A-B. Cell Cycle Delay After Overnight Exposure to Compound 1205. Histograms depicting relative DNA content in HT1080 cells under normoxic conditions after treatment with Compound 1205 compared to vehicle. FIG. 3A. Histogram showing the effect of treatment with Compound 1205 at 10 nm. FIG. 3B. Histogram showing the effect of treatment with vehicle.

FIG. 4A-F. Treatment of BrdU labeled HT1080 cells with increasing doses of Compound #10. FIG. 4A. The effect of DMSO control on percentage of cells residing in S-phase.

FIGS. 4B-F. The effect of increasing concentration of Compound #10 at 1 nm, 3 nm, 10 nm, 30 nm and 100 nm, respectively, on percentage of cells residing in S-phase.

FIG. 5A-B. FIG. 5A. The percentage of cells incorporating BrdU. FIG. 5B. The relative level of BrdU at each Compound #10 concentration.

FIG. 6A-B-C. BrdU Histogram and Quantification: FIG. 6(A). Histograms of DNA content demonstrating that the cell cycle distribution for HT1080 spheroids treated for 24 hours is not affected by exposure to Compound #10; FIG. 6(A)(i). Data.001 shows the control results; FIG. 6(A)(ii). Data.002 shows the results of exposure at 5 nm Compound #10; and, FIG. 6(A)(iii). Data.003 shows the results of exposure at 50 nm Compound #10. FIG. 6(B). BrdU quantification indicating the fraction of cells actively synthesizing DNA; FIG. 6(B)(i). The effect of the DMSO control; FIG. 6(B)(ii). Represents the Data.001 results; and, FIG. 6(B)(iii). Represents the Data.003 results. FIG. 6(C) A graphical representation of the percentage of cells that incorporated BrdU (i.e., the cells in S-phase) after treatment with Compound #10 at various concentrations.

FIG. 7A-B-C. BrdU Histogram and Quantification: FIG. 7(A). Histograms of DNA content demonstrating that the cell cycle distribution for HT1080 spheroids treated for 48 hours is not affected by exposure to Compound #10; FIG. 7(A)(i). Data.004 shows the control results; FIG. 7(A)(ii). Data.005 shows the results of exposure at 10 nm Compound #10; and, FIG. 7(A)(iii). Data.006 shows the results of exposure at 50 nm Compound #10. FIG. 7(B). BrdU quantification indicating the fraction of cells actively synthesizing DNA; FIG. 7(B)(i). Represents the Data.004 results; FIG. 7(B)(ii). Represents the Data.005 results; and, FIG. 7(B)(iii). Represents the Data.006 results. FIG. 7(C) A graphical representation of the percentage of cells that incorporated BrdU (i.e., the cells in S-phase) after treatment with Compound #10 at various concentrations.

### 6. DETAILED DESCRIPTION

Encompassed herein are Compounds capable of inhibiting the production of viral RNA or DNA or production of one or more viral proteins or production of one or more virus induced cytopathic effects. Also encompassed herein are methods of treating viral infections using the Compounds as well as methods of using the Compounds to inhibit or reduce viral replication and/or production of viral RNA or DNA, viral protein or virus induced cytopathic effects.

6.1 Compounds

In one embodiment, provided herein are Compounds having Formula (I):

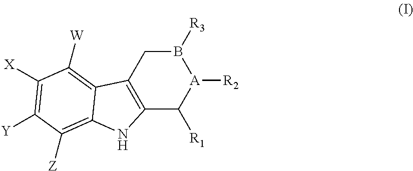

or a pharmaceutically acceptable salt, racemate, tautomer or stereoisomer thereof,

wherein,
W is hydrogen; or halogen;
X is hydrogen; optionally substituted C₁ to C₈ alkyl; hydroxyl; halogen; thioether; sulfinyl;
alkylsulfinyl; sulfonyl; alkylsulfonyl; cyano; or optionally substituted C₁ to C₈ alkoxy;
Y is hydrogen; optionally substituted C₁ to C₈ alkyl; or halogen;
Z is hydrogen; or C₁ to C₈ alkyl;
A is CH or N;
B is CH or N, with the proviso that at least one of A or B is N, and that when A is N, B is CH;
R₁ is hydroxyl; optionally substituted C₁ to C₈ alkyl; C₂ to C₈ alkenyl; C₂ to C₈ alkynyl; optionally substituted heterocyclyl; optionally substituted heteroaryl; or optionally substituted aryl;
R₂ is hydrogen; hydroxyl; halogen; optionally substituted heteroaryl; optionally substituted C₁ to C₈ alkyl, phosphonic acid; phosphonate; —C(O)—R_{c}; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; optionally substituted amino; optionally substituted aryl; —C(O)—Rₙ; optionally substituted heterocyclyl; heteroaryl; thiazoleamino; optionally substituted C₁ to C₈ alkyl; cycloalkyl; or optionally substituted C₂ to C₈ alkenyl;
R_{d} is at each occurrence independently hydrogen; C₂ to C₈ alkenyl; C₂ to C₈ alkynyl; optionally substituted aryl; cycloalkyl; or optionally substituted C₁ to C₈ alkyl;
Rₑ is hydrogen; optionally substituted C₁ to C₈ alkyl; optionally substituted cycloalkyl; or optionally substituted aryl;
R_{f} is optionally substituted C₁ to C₈ alkyl;
Rₙ is hydroxyl; C₁ to C₈ alkoxy; amino; optionally substituted aryl;
R₃ is hydrogen; or —C(O)—R_{g}; and
R_{g} is hydroxyl; alkylcarbonyl, optionally substituted amino; optionally substituted heteroaryl; or optionally substituted heterocyclyl.

In a particular embodiment, compounds of Formula (I) are those wherein:
X is optionally substituted C₁ to C₈ alkyl, wherein the optional substituents are one or more independently selected halogen substituents;
X is optionally substituted C₁ to C₈ alkoxy, wherein the optional substituents are one or more substituents independently selected from aryl or C₁ to C₈ alkyl;
Y is optionally substituted C₁ to C₈ alkyl, wherein the optional substituents are one or more independently selected halogen substituents;
R₁ is optionally substituted C₁ to C₈ alkyl, wherein the optional substituents are one or more substituents independently selected from thioether, heteroaryl or optionally substituted aryl, wherein the optional aryl substituents are one or more independently selected Rₒ substituents;
R₁ is optionally substituted heterocyclyl, wherein the optional substituents are one or more substituents independently selected from halogen, oxo, amino, alkylamino, acetamino, thiol or thioether;
R₁ is optionally substituted heteroaryl, wherein the optional substituents are one or more substituents independently selected from halogen, oxo, amino, alkylamino, acetamino, thiol, C₁ to C₈ alkoxy or thioether;
R₁ is optionally substituted aryl, wherein the optional substituents are one or more independently selected Rₒ substituents;
Rₒ is hydrogen; halogen; cyano; nitro; optionally substituted sulfonyl; optionally substituted amino; —C(O)—N(R_{b})₂; heterocyclyl; heteroaryl; optionally substituted C₁ to C₈ alkyl; —C(O)—Rₙ; or —ORₐ;
Rₐ is hydrogen; C₂ to C₈ alkenyl; C₂ to C₈ alkynyl; —C(O)—Rₙ; —C(O)O—R_{b}; —C(O)—NH—R_{b}; cycloalkyl; aryl; optionally substituted heteroaryl; optionally substituted heterocyclyl; or, optionally substituted C₁ to C₈ alkyl;
R_{b} is hydrogen; hydroxyl; amino; optionally substituted monoalkylamino; optionally substituted dialkylamino; C₁ to C₈ alkoxy; C₂ to C₈ alkenyl; C₂ to C₈ alkynyl; optionally substituted aryl; heteroaryl; optionally substituted heterocyclyl; or optionally substituted C₁ to C₈ alkyl;
R₂ is optionally substituted heteroaryl, wherein the optional substituents are one or more substituents independently selected from hydroxyl, heteroaryl, —C(O)—Rₙ—C(O)O—R_{d}, —C(O)—N(R_{d}R_{d}), optionally substituted C₁ to C₈ alkyl or optionally substituted aryl, wherein the optional C₁ to C₈ alkyl substituents are one or more substituents independently selected from halogen, aryl or —C(O)—R_{c}; and, wherein the optional aryl substituents are one or more substituents independently selected from halogen or C₁ to C₈ alkoxy;
R₂ is optionally substituted C₁ to C₈ alkyl, wherein the optional substituents are one or more substituents independently selected from hydroxyl, C₁ to C₈ alkoxy, heterocyclyl, heteroaryl or aryl;
R_{c} is optionally substituted amino, wherein the optional substituents are one or more substituents independently selected from C₁ to C₈ alkyl or aryl;
R_{c} is optionally substituted aryl, wherein the optional substituents are one or more substituents independently selected from halogen, haloalkyl, hydroxyl, C₁ to C₈ alkoxy, or C₁ to C₈ alkyl;
R_{c} is optionally substituted heterocyclyl, wherein the optional substituents are one or more independently selected —C(O)—Rₙ substituents;
R_{c} is optionally substituted C₁ to C₈ alkyl, wherein the optional substituents are one or more substituents independently selected from halogen, C₁ to C₈ alkoxy, phenyloxy, aryl, —C(O)—Rₙ, —O—C(O)—Rₙ, hydroxyl, or optionally substituted amino, wherein the optional amino substituents are one or more substituents independently selected from —C(O)O—Rₙ or —C(O)—Rₙ;
R_{c} is optionally substituted C₂ to C₈ alkenyl, wherein the optional substituents are one or more independently selected aryl substituents;
R_{d} is at each occurrence optionally substituted aryl, wherein the optional substituents are one or more substituents independently selected from halogen, cyano, nitro, C₁ to C₈ alkyl, —C(O)O—Rₑ, or —ORₑ;
R_{d} is at each occurrence optionally substituted C₁ to C₈ alkyl, wherein the optional substituents are one or more substituents independently selected from halogen, C₁ to C₈ alkyl, C₁ to C₈ alkoxy, optionally substituted cycloalkyl, phenyloxy, optionally substituted aryl, heteroaryl, —C(O)—Rₙ, —C(O)O—Rₙ, or hydroxyl, wherein the optional aryl substituents are one or more substituents independently selected from C₁ to C₈ alkyl, C₁ to C₈ alkoxy, cyano, halogen or haloalkyl; and, wherein the optional cycloalkyl substituents are one or more independently selected C₁ to C₈ alkyl substituents;
Rₑ is optionally substituted C₁ to C₈ alkyl, wherein the optional substituents are one or more substituents independently selected from halogen, —C(O)—Rₙ or C₁ to C₈ alkoxy;
Rₑ is optionally substituted cycloalkyl, wherein the optional substituents are one or more independently selected oxo substituents;
Rₑ is optionally substituted aryl, wherein the optional substituents are one or more substituents independently selected from halogen or C₁ to C₈ alkoxy;
R_{f} is optionally substituted C₁ to C₈ alkyl, wherein the optional substituents are one or more substituents independently selected from halogen, hydroxyl, optionally substituted C₁ to C₈ alkoxy, cyano, optionally substituted aryl, or —C(O)—Rₙ, wherein the optional C₁ to C₈ alkoxy substituents are one or more C₁ to C₈ alkoxy substituents; and, wherein the optional aryl substituents are one or more substituents independently selected from halogen, hydroxyl, C₁ to C₈ alkoxy, cyano, or C₁ to C₈ alkyl;
R_{g} is optionally substituted amino, wherein the optional substituents are one or more substituents independently selected from cycloalkyl or heteroaryl;
R_{g} is optionally substituted heteroaryl, wherein the optional substituents are one or more independently selected amino substituents;
R_{g} is optionally substituted heterocyclyl, wherein the optional substituents are one or more substituents independently selected from C₁ to C₈ alkyl, aryl or —C(O)—Rₙ; and,
Rₙ is optionally substituted aryl, wherein the optional substituents are one or more substituents independently selected from halogen, C₁ to C₈ alkyl or C₁ to C₈ alkoxy.

In a particular embodiment, compounds of Formula (I) are those wherein:
Rₒ is optionally substituted sulfonyl, wherein the optional substituents are one or more substituents independently selected from C₁ to C₈ alkyl or heterocyclyl;
Rₒ is optionally substituted amino, wherein the optional substituents are one or more substituents independently selected from C₁ to C₆ alkyl, —C(O)—R_{b}, —C(O)O—R_{b}, sulfonyl, alkylsulfonyl or optionally substituted heterocyclyl, wherein the optional heterocyclyl substituents are one or more independently selected —C(O)O—Rₙ, substituents;
Rₒ is optionally substituted C₁ to C₈ alkyl, wherein the optional substituents are one or more substituents independently selected from hydroxyl, halogen, optionally substituted amino or optionally substituted heterocyclyl, wherein the optional amino and heterocyclyl substituents are one or more independently selected optionally substituted C₁ to C₈ alkyl substituents, wherein the optional C₁ to C₈ alkyl substituents are one or more substituents independently selected from C₁ to C₈ alkoxy, amino, alkylamino, or heterocyclyl;
Rₐ is optionally substituted heteroaryl, wherein the optional substituents are one or more substituents independently selected from halogen, amino, or optionally substituted C₁ to C₈ alkyl, wherein the optional C₁ to C₈ alkyl substituents are one or more substituents independently selected from alkylsulfonyl or optionally substituted heterocyclyl, wherein the optional heterocyclyl substituents are one or more independently selected C₁ to C₈ alkyl substituents;
Rₐ is optionally substituted heterocyclyl, wherein the optional substituents are one or more substituents independently selected from hydroxyl, acetate, amino, heteroaryl, trimethylsilyl-C₁ to C₈ alkyl-sulfonyl or optionally substituted C₁ to C₈ alkyl, wherein the optional C₁ to C₈ alkyl substituents are one or more substituents independently selected from hydroxyl, heterocyclyl, aryl, heteroaryl, —C(O)O—R_{b}, sulfonyl, or C₁ to C₈ alkyl-sulfonyl;
Rₐ is optionally substituted C₁ to C₈ alkyl, wherein the optional substituents are one or more substituents independently selected from hydroxyl, halogen, cyano, optionally substituted C₁ to C₈ alkoxy, optionally substituted amino, optionally substituted monoalkylamino, optionally substituted dialkylamino, optionally substituted acetamino, guanidino, optionally substituted sulfonyl, thioether, optionally substituted sulfonamide, —C(O)—R_{b}, —C(O)O—R_{b}, —OR_{g}, aryl, optionally substituted heterocyclyl or optionally substituted heteroaryl,
   wherein the optional C₁ to C₈ alkoxy substituents are one or more substituents independently selected from C₁ to C₈ alkoxy or heterocyclyl,
   wherein the optional amino substituents are one or more substituents independently selected from optionally substituted alkoxycarbonyl, optionally substituted alkylcarbonyl, alkylsulfonyl, cycloalkyl, cycloalkylcarbonyl, heteroarylcarbonyl, cycloalkylsulfonyl, optionally substituted heteroaryl or optionally substituted sulfonyl, wherein the optional heteroaryl, alkoxycarbonyl, alkylcarbonyl, and sulfonyl substituents are one or more substituents independently selected from C₁ to C₈ alkyl or C₁ to C₈ alkoxy,
   wherein the optional monoalkylamino or dialkylamino substituents are one or more substituents substituted on alkyl independently selected from hydroxyl, C₁ to C₈ alkoxy, amino, heterocyclyl, dialkylamino or heteroaryl optionally substituted with C₁ to C₈ alkyl,
   wherein the optional acetamino substituents are one or more substituents independently selected from C₁ to C₈ alkyl or optionally substituted C₁ to C₈ alkoxy, wherein the optional C₁ to C₈ alkoxy substituents are one or more substituents independently selected from C₁ to C₈ alkoxy, cycloalkyl, heteroaryl, sulfonyl or alkylsulfonyl,
   wherein the optional sulfonyl substituents are one or more independently selected C₁ to C₈ alkyl substituents, and
   wherein the optional sulfonamide substituents are one or more substituents independently selected from C₁ to C₈ alkyl or cycloalkyl,
   wherein the optional heterocyclyl substituents are one or more substituents independently selected from imino, —C(O)—R_{f}, —C(O)O—R_{f}, oxo or optionally substituted C₁ to C₈ alkyl, wherein the optional C₁ to C₈ alkyl substituents are one or more independently selected hydroxyl substituents, and,
   wherein the optional heteroaryl substituents are one or more substituents independently selected from C₁ to C₈ alkyl, —C(O)—NH—R_{b}, imino, amino, cyano or optionally substituted heterocyclyl, wherein the optional heterocyclyl substituents are one or more acetate or hydroxyl substituents;
R_{b} is optionally substituted monoalkylamino or optionally substituted dialkylamino, wherein one or more substituents substituted on alkyl are independently selected from hydroxyl, amino, alkylamino, C₁ to C₈ alkoxy, optionally substituted heterocyclyl or optionally substituted heteroaryl, wherein the optional heterocyclyl substituents are one or more substituents independently selected from C₁ to C₈ alkyl, oxo or —C(O)O—Rₙ, and, wherein the optional heteroaryl substituents are one or more independently selected C₁ to C₈ alkyl substituents;
R_{b} is optionally substituted aryl, wherein the optional substituents are one or more substituents independently selected from halogen or C₁ to C₈ alkoxy;
R_{b} is optionally substituted heterocyclyl, wherein the optional substituents are one or more substituents independently selected from acetamino, —C(O)O—Rₙ, heterocyclyl or optionally substituted C₁ to C₈ alkyl, wherein the optional C₁ to C₈ alkyl substituents are one or more substituents independently selected from hydroxyl, C₁ to C₈ alkoxy, amino, monoalkylamino or dialkylamino; and.
R_{b} is optionally substituted C₁ to C₈ alkyl, wherein the optional substituents are one or more substituents independently selected from C₁ to C₈ alkoxy, aryl, optionally substituted amino, —C(O)—Rₙ, or optionally substituted heterocyclyl, wherein the optional amino and heterocyclyl substituents are one or more substituents independently selected from C₁ to C₈ alkyl, oxo, or —C(O)O—Rₙ.

In a particular embodiment, compounds of Formula (I) are those wherein:
W is hydrogen; or halogen;
X is hydrogen; C₁ to C₈ alkyl optionally substituted with one or more halogen substituents; hydroxyl; halogen; thioether; sulfinyl; sulfonyl; cyano; or C₁ to C₈ alkoxy optionally substituted with aryl or C₁ to C₈ alkyl;
Y is hydrogen; C₁ to C₈ alkyl optionally substituted with one or more halogen substituents; or halogen;
Z is hydrogen; or C₁ to C₈ alkyl;
A is CH or N;
B is CH or N, with the proviso that at least one of A or B is N, and that when A is N, B is CH;
R₁ is hydroxyl; C₁ to C₈ alkyl optionally substituted with thioether, heteroaryl, or optionally substituted aryl; C₂ to C₈ alkenyl; C₂ to C₈ alkynyl; heterocyclyl optionally substituted with one or more substituents independently selected from halogen, oxo, amino, alkylamino, acetamino, thiol, or thioether; heteroaryl optionally substituted with one or more substituents independently selected from halogen, oxo, amino, alkylamino, acetamino, thiol, C₁ to C₈ alkoxy or thioether; or aryl optionally substituted with one or more independently selected Rₒ substituents;
Rₒ is a halogen; cyano; nitro; sulfonyl optionally substituted with C₁ to C₈ alkyl or heterocyclyl; amino optionally substituted with C₁ to C₆ alkyl, —C(O)—R_{b}, —C(O)O—R_{b}, sulfonyl, alkylsulfonyl or optionally substituted heterocyclyl; —C(O)—NH—R_{b}; heterocyclyl; heteroaryl; C₁ to C₈ alkyl optionally substituted with one or more substituents independently selected from hydroxyl, halogen, optionally substituted amino or optionally substituted heterocyclyl; —C(O)—Rₙ; or —ORₐ;
Rₐ is hydrogen; C₂ to C₈ alkenyl; C₂ to C₈ alkynyl; —C(O)—Rₙ; —C(O)O—R_{b}; —C(O)—NH—R_{b}; aryl; heteroaryl optionally substituted with halogen, amino, optionally substituted C₁ to C₈ alkyl; heterocyclyl optionally substituted with hydroxyl, optionally substituted C₁ to C₈ alkyl; C₁ to C₈ alkyl optionally substituted with one or more substituents independently selected from hydroxyl, halogen, cyano, optionally substituted C₁ to C₈ alkoxy, optionally substituted amino, optionally substituted monoalkylamino, optionally substituted dialkylamino, optionally substituted acetamino, sulfonyl, thioether, optionally substituted sulfonamide, —C(O)—R_{b}, —C(O)O—R_{b}, —OR_{g}, aryl, optionally substituted heterocyclyl, or optionally substituted heteroaryl;
R_{b} is hydroxyl; amino; or monoalkylamino or dialkylamino, wherein alkyl is independently optionally substituted with one or more substituents independently selected from hydroxyl, amino, alkylamino, C₁ to C₈ alkoxy, optionally substituted heterocyclyl; C₁ to C₈ alkoxy; C₂ to C₈ alkenyl; C₂ to C₈ alkynyl; aryl optionally substituted with one or more substituents independently selected from halogen or C₁ to C₈ alkoxy; heteroaryl; heterocyclyl optionally substituted with one or more substituents independently selected from acetamino, —C(O)O—Rₙ, heterocyclyl, or optionally substituted C₁ to C₈ alkyl; or C₁ to C₈ alkyl optionally substituted with one or more substituents independently selected from C₁ to C₈ alkoxy, aryl, optionally substituted amino, —C(O)O—Rₙ, or optionally substituted heterocyclyl;
R₂ is hydrogen; hydroxyl; halogen; heteroaryl optionally substituted with hydroxyl, optionally substituted C₁ to C₈ alkyl, optionally substituted aryl, heteroaryl, —C(O)O—R_{d}, —C(O)—N(R_{d}R_{d}); C₁ to C₈ alkyl optionally substituted with hydroxyl, C₁ to C₈ alkoxy, heterocyclyl, heteroaryl, or aryl; —C(O)—R_{c}; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; amino optionally substituted with one or more substituents independently selected from C₁ to C₈ alkyl or aryl; aryl optionally substituted with one or more substituents independently selected from halogen, haloalkyl, hydroxyl, C₁ to C₈ alkoxy, or C₁ to C₈ alkyl; —C(O)—Rₙ; heterocyclyl optionally substituted with —C(O)—Rₙ; heteroaryl; thiazoleamino; C₁ to C₈ alkyl optionally substituted with one or more substituents independently selected from halogen, C₁ to C₈ alkoxy, phenyloxy, aryl, —C(O)—Rₙ, —O—C(O)—Rₙ, hydroxyl, or optionally substituted amino; cycloalkyl; or C₂ to C₈ alkenyl optionally substituted with aryl;
R_{d} is at each occurrence independently hydrogen; C₂ to C₈ alkenyl; C₂ to C₈ alkynyl; aryl optionally substituted with one or more substituents independently selected from halogen, nitro, C₁ to C₈ alkyl, —C(O)O—Rₑ, or —ORₑ; cycloalkyl; or C₁ to C₈ alkyl optionally substituted with one or more substituents independently selected from halogen, C₁ to C₈ alkyl, C₁ to C₈ alkoxy, cycloalkyl, phenyloxy, optionally substituted aryl, heteroaryl, —C(O)—Rₙ, —C(O)O—Rₙ, or hydroxyl;
Rₑ is hydrogen; C₁ to C₈ alkyl optionally substituted with one or more substituents independently selected from halogen, —C(O)—Rₙ or C₁ to C₈ alkoxy; cycloalkyl optionally substituted with oxo; or aryl optionally substituted with one or more substituents independently selected from halogen or C₁ to C₈ alkoxy;
R_{f} is C₁ to C₈ alkyl optionally substituted with one or more substituents independently selected from halogen, hydroxyl, optionally substituted C₁ to C₈ alkoxy, cyano, optionally substituted aryl, or —C(O)—Rₙ;
Rₙ is hydroxyl; C₁ to C₈ alkoxy; amino; or aryl optionally substituted with halogen or C₁ to C₈ alkyl;
R₃ is hydrogen; or —C(O)—R_{g}; and
R_{g} is hydroxyl; amino optionally substituted with cycloalkyl or heteroaryl; heteroaryl optionally substituted with amino; or heterocyclyl optionally substituted with —C(O)—Rₙ.

In a more particular embodiment, compounds of Formula (I) are those wherein:
W is hydrogen; or halogen;
X is hydrogen; C₁ to C₈ alkyl optionally substituted with one or more halogen substituents; hydroxyl; halogen; thioether; sulfinyl; sulfonyl; cyano; or C₁ to C₈ alkoxy optionally substituted with aryl or C₁ to C₈ alkyl;
Y is hydrogen; C₁ to C₈ alkyl optionally substituted with one or more halogen substituents; or halogen;
Z is hydrogen; or C₁ to C₈ alkyl;
A is CH or N;
B is CH or N, with the proviso that at least one of A or B is N, and that when A is N, B is CH;
R₁ is hydroxyl; C₁ to C₈ alkyl optionally substituted with thioether, heteroaryl, or aryl optionally substituted with one or more independently selected Rₒ substituents; C₂ to C₈ alkenyl; C₂ to C₈ alkynyl; heterocyclyl optionally substituted with one or more substituents independently selected from halogen, oxo, amino, alkylamino, acetamino, thiol, or thioether; heteroaryl optionally substituted with one or more substituents independently selected from halogen, oxo, amino, alkylamino, acetamino, thiol, C₁ to C₈ alkoxy or thioether; or aryl optionally substituted with one or more independently selected Rₒ substituents;
Rₒ is a halogen; cyano; nitro; sulfonyl optionally substituted with C₁ to C₈ alkyl or heterocyclyl; amino optionally substituted with C₁ to C₆ alkyl, —C(O)—R_{b}, —C(O)O—R_{b}, sulfonyl, alkylsulfonyl or heterocyclyl optionally substituted with —C(O)O—Rₙ; —C(O)—NH—R_{b}; heterocyclyl; heteroaryl; C₁ to C₈ alkyl optionally substituted with one or more substituents independently selected from hydroxyl, halogen, amino or heterocyclyl, wherein amino and heterocyclyl are optionally substituted with one or more C₁ to C₈ alkyl substituents optionally substituted with one or more substituents independently selected from C₁ to C₈ alkoxy, amino, alkylamino, or heterocyclyl; —C(O)—Rₙ; or —ORₐ;
Rₐ is hydrogen; C₂ to C₈ alkenyl; C₂ to C₈ alkynyl; —C(O)—Rₙ; —C(O)O—R_{b}; —C(O)—NH—R_{b}; cycloalkyl; aryl; heteroaryl optionally substituted with halogen, amino, or C₁ to C₈ alkyl optionally substituted with heterocyclyl or alkylsulfonyl, wherein heterocyclyl is optionally substituted with C₁ to C₈ alkyl; heterocyclyl optionally substituted with hydroxyl, C₁ to C₈ alkyl optionally substituted with hydroxyl, heterocyclyl, aryl, heteroaryl, —C(O)O—R_{b}, sulfonyl, C₁ to C₈ alkyl-sulfonyl or silyl-C₁ to C₈ alkyl-sulfonyl; C₁ to C₈ alkyl optionally substituted with one or more substituents independently selected from hydroxyl, halogen, cyano, C₁ to C₈ alkoxy, amino, monoalkylamino, dialkylamino, acetamino, sulfonyl, thioether, sulfonamide, —C(O)—R_{b}, —C(O)O—R_{b}, —OR_{g}, aryl, heterocyclyl, or heteroaryl, wherein the amino is optionally substituted with cycloalkyl, the alkyl of the monoalkylamino or dialkylamino is independently optionally substituted with one or more substituents independently selected from hydroxyl, C₁ to C₈ alkoxy, imino, amino, heterocyclyl, dialkylamino or heteroaryl optionally substituted with C₁ to C₈ alkyl, wherein the heteroaryl is optionally substituted with one or more substituents independently selected from —C(O)—NH—R_{b}, amino, cyano or heterocyclyl optionally substituted with one or more acetate or hydroxyl substituents, wherein the acetamino is optionally substituted with C₁ to C₈ alkyl, C₁ to C₈ alkoxy optionally substituted with C₁ to C₈ alkoxy, cycloalkyl, heteroaryl, sulfonyl, or alkylsulfonyl, wherein the heterocyclyl is optionally substituted with imino, —C(O)—Rₙ, —C(O)O—Rₙ, oxo or C₁ to C₈ alkyl optionally substituted with hydroxyl, further wherein the C₁ to C₈ alkoxy is optionally substituted with heterocyclyl, further wherein the sulfonamide is optionally substituted with C₁ to C₈ alkyl or cycloalkyl, further wherein the amino is optionally substituted with alkoxycarbonyl, alkylsulfonyl, cycloalkylsulfonyl, imidazole, isothiazole, pyrazole, pyridine, pyrazine, pyrimidine, pyrrole, thiazole, isoxazole, triazine or sulfonyl substituted with C₁ to C₈ alkyl, wherein pyridine, isoxazole, and thiazole are each optionally substituted with C₁ to C₈ alkyl;
R_{b} is hydroxyl; amino; or monoalkylamino or dialkylamino, wherein alkyl is independently optionally substituted with one or more substituents independently selected from hydroxyl, amino, alkylamino, C₁ to C₈ alkoxy, heterocyclyl optionally substituted with one or more substituents independently selected from C₁ to C₈ alkyl, oxo, —C(O)O—Rₙ, or heteroaryl optionally substituted with C₁ to C₈ alkyl; C₁ to C₈ alkoxy; C₂ to C₈ alkenyl; C₂ to C₈ alkynyl; aryl optionally substituted with one or more substituents independently selected from halogen or C₁ to C₈ alkoxy; heteroaryl; heterocyclyl optionally substituted with one or more substituents independently selected from acetamino, —C(O)O—R, heterocyclyl, or C₁ to C₈ alkyl optionally substituted with hydroxyl, C₁ to C₈ alkoxy, amino, monoalkylamino or dialkylamino; or C₁ to C₈ alkyl optionally substituted with one or more substituents independently selected from C₁ to C₈ alkoxy, aryl, amino, —C(O)O—R, or heterocyclyl, wherein the amino and heterocyclyl are optionally substituted with one or more substituents independently selected from C₁ to C₈ alkyl, oxo, or —C(O)O—Rₙ;
R₂ is hydrogen; hydroxyl; halogen; heteroaryl optionally substituted with hydroxyl, C₁ to C₈ alkyl optionally substituted with halogen, aryl or —C(O)—R_{c}, aryl optionally substituted with halogen or C₁ to C₈ alkoxy, heteroaryl, —C(O)O—R_{d}, —C(O)—N(R_{d}R_{d}); C₁ to C₈ alkyl optionally substituted with hydroxyl, C₁ to C₈ alkoxy, heterocyclyl, heteroaryl, or aryl; —C(O)—Rₑ; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
Rₑ is hydrogen; amino optionally substituted with one or more substituents independently selected from C₁ to C₈ alkyl or aryl; aryl optionally substituted with one or more substituents independently selected from halogen, haloalkyl, hydroxyl, C₁ to C₈ alkoxy, or C₁ to C₈ alkyl; —C(O)—Rₙ; heterocyclyl optionally substituted with —C(O)—Rₙ; heteroaryl; thiazoleamino; C₁ to C₈ alkyl optionally substituted with one or more substituents independently selected from halogen, C₁ to C₈ alkoxy, phenyloxy, aryl, —C(O)—Rₙ, —O—C(O)—Rₙ, hydroxyl, or amino optionally substituted with —C(O)O—Rₙ, or —C(O)—Rₙ; cycloalkyl; or C₂ to C₈ alkenyl optionally substituted with aryl;
R_{d} is at each occurrence independently hydrogen; C₂ to C₈ alkenyl; C₂ to C₈ alkynyl; aryl optionally substituted with one or more substituents independently selected from halogen, nitro, C₁ to C₈ alkyl, —C(O)O—Rₑ, or —ORₑ; cycloalkyl; or C₁ to C₈ alkyl optionally substituted with one or more substituents independently selected from halogen, C₁ to C₈ alkyl, C₁ to C₈ alkoxy, cycloalkyl, phenyloxy, aryl, heteroaryl, —C(O)—Rₙ, —C(O)O—Rₙ, or hydroxyl, wherein the aryl is optionally substituted with one or more substituents independently selected from C₁ to C₈ alkyl, C₁ to C₈ alkoxy, cyano, halogen or haloalkyl;
Rₑ is hydrogen; C₁ to C₈ alkyl optionally substituted with one or more substituents independently selected from halogen, —C(O)—Rₙ or C₁ to C₈ alkoxy; cycloalkyl optionally substituted with oxo; or aryl optionally substituted with one or more substituents independently selected from halogen or C₁ to C₈ alkoxy;
R_{f} is C₁ to C₈ alkyl optionally substituted with one or more substituents independently selected from halogen, hydroxyl, C₁ to C₈ alkoxy, cyano, aryl, or —C(O)—Rₙ, wherein the C₁ to C₈ alkoxy is optionally substituted with one or more C₁ to C₈ alkoxy substituents and the aryl is optionally substituted with one or more substituents independently selected from halogen, hydroxyl, C₁ to C₈ alkoxy, cyano, or C₁ to C₈ alkyl;
Rₙ is hydroxyl; C₁ to C₈ alkoxy; amino; or aryl optionally substituted with halogen or C₁ to C₈ alkyl;
R₃ is hydrogen; or —C(O)—R_{g}; and
R_{g} is hydroxyl; amino optionally substituted with cycloalkyl or heteroaryl; heteroaryl optionally substituted with amino; or heterocyclyl, wherein the heterocyclyl is optionally substituted with —C(O)—Rₙ.

In certain embodiments, compounds provided herein, including Compounds having Formula (I), do not include one or more of the following compounds:
(R)-1-(benzo[d][1,3]dioxol-5-yl)-2,3,4,9-tetrahydro-1H-pyrido[3,4-b]indole,
1-(benzo[d][1,3]dioxol-5-yl)-N-benzyl-3,4-dihydro-1H-pyrido[3,4-b]indole-2(9H)-carbothioamide,
(R)-1-(benzo[d][1,3]dioxol-5-yl)-N-benzyl-3,4-dihydro-1H-pyrido[3,4-b]indole-2(9H)-carbothioamide,
1-phenyl-2,3,4,9-tetrahydro-1H-pyrido[3,4-b]indole,
(R)-1-(benzo[d][1,3]dioxol-5-yl)-N-benzyl-3,4-dihydro-1H-pyrido[3,4-b]indole-2(9H)-carboxamide,
N-benzyl-1-phenyl-3,4-dihydro-1H-pyrido[3,4-b]indole-2(9H)-carboxamide,
N,1-diphenyl-3,4-dihydro-1H-pyrido[3,4-b]indole-2(9H)-carboxamide,
N-(naphthalen-1-yl)-1-phenyl-3,4-dihydro-1H-pyrido[3,4-b]indole-2(9H)-carboxamide,
1-(benzo[d][1,3]dioxol-5-yl)-N-cyclohexyl-3,4-dihydro-1H-pyrido[3,4-b]indole-2(9H)-carboxamide,
1-(benzo[d][1,3]dioxol-5-yl)-N-phenyl-3,4-dihydro-1H-pyrido[3,4-b]indole-2(9H)-carboxamide,
1-(3-chloro-4-methoxyphenyl)-N-phenyl-3,4-dihydro-1H-pyrido[3,4-b]indole-2(9H)-carboxamide,
(R)-1-(benzo[d][1,3]dioxol-5-yl)-N—((R)-1-phenylethyl)-3,4-dihydro-1H-pyrido[3,4-b]indole-2(9H)-carboxamide,
(R)-1-(benzo[d][1,3]dioxol-5-yl)-N—((S)-1-phenylethyl)-3,4-dihydro-1H-pyrido[3,4-b]indole-2(9H)-carboxamide,
(R)-1-(benzo[d][1,3]dioxol-5-yl)-N-benzoyl-3,4-dihydro-1H-pyrido[3,4-b]indole-2(9H)-carboxamide,
(R)—N-(1-(benzo[d][1,3]dioxol-5-yl)-2,3,4,9-tetrahydro-1H-pyrido[3,4-b]indole-2-carbonothioyl)benzamide,
benzyl 1-(benzo[d][1,3]dioxol-5-yl)-3,4-dihydro-1H-pyrido[3,4-b]indole-2(9H)-carboxylate,
(R)-benzyl 1-(benzo[d][1,3]dioxol-5-yl)-3,4-dihydro-1H-pyrido[3,4-b]indole-2(9H)-carboxylate,
methyl 1-phenyl-3,4-dihydro-1H-pyrido[3,4-b]indole-2(9H)-carboxylate,
methyl 5-oxo-5-(1-phenyl-3,4-dihydro-1H-pyrido[3,4-b]indol-2(9H)-yl)pentanoate,
5-(1-(3-chloro-4-methoxyphenyl)-3,4-dihydro-1H-pyrido[3,4-b]indol-2(9H)-yl)-5-oxopentanoic acid,
5-(1-(benzo[d][1,3]dioxol-5-yl)-3,4-dihydro-1H-pyrido[3,4-b]indol-2(9H)-yl)-5-oxopentanoic acid,
3-(2-aminophenyl)-1-(1-(benzo[d][1,3]dioxol-5-yl)-3,4-dihydro-1H-pyrido[3,4-b]indol-2(9H)-yl)propan-1-one,
(R)-1-(benzo[d][1,3]dioxol-5-yl)-N-(2-chlorobenzyl)-3,4-dihydro-1H-pyrido[3,4-b]indole-2(9H)-carbothioamide,
(R)-1-(benzo[d][1,3]dioxol-5-yl)-N-(2,4-dichlorobenzyl)-3,4-dihydro-1H-pyrido[3,4-b]indole-2(9H)-carbothioamide,
(R)-1-(benzo[d][1,3]dioxol-5-yl)-N-(2-fluorobenzyl)-3,4-dihydro-1H-pyrido[3,4-b]indole-2(9H)-carbothioamide,
(R)-1-(benzo[d][1,3]dioxol-5-yl)-N—((S)-1-phenylethyl)-3,4-dihydro-1H-pyrido[3,4-b]indole-2(9H)-carbothioamide,
(R)-4-((1-(benzo[d][1,3]dioxol-5-yl)-2,3,4,9-tetrahydro-1H-pyrido[3,4-b]indole-2-carbothioamido)methyl)benzoic acid,
(R)-methyl 4-((1-(benzo[d][1,3]dioxol-5-yl)-2,3,4,9-tetrahydro-1H-pyrido[3,4-b]indole-2-carbothioamido)methyl)benzoate,
(R)-3-((1-(benzo[d][1,3]dioxol-5-yl)-2,3,4,9-tetrahydro-1H-pyrido[3,4-b]indole-2-carbothioamido)methyl)benzoic acid,
(R)-methyl 3-((1-(benzo[d][1,3]dioxol-5-yl)-2,3,4,9-tetrahydro-1H-pyrido[3,4-b]indole-2-carbothioamido)methyl)benzoate,
(R)-1-(benzo[d][1,3]dioxol-5-yl)-N-(4-chloro-3-(trifluoromethyl)phenyl)-3,4-dihydro-1H-pyrido[3,4-b]indole-2(9H)-carbothioamide,
(R)-1-(benzo[d][1,3]dioxol-5-yl)-N-(2-(trifluoromethyl)phenyl)-3,4-dihydro-1H-pyrido[3,4-b]indole-2(9H)-carbothioamide,
(R)-1-(benzo[d][1,3]dioxol-5-yl)-N-(3-fluorobenzyl)-3,4-dihydro-1H-pyrido[3,4-b]indole-2(9H)-carbothioamide,
(R)-1-(benzo[d][1,3]dioxol-5-yl)-N-(4-chlorobenzyl)-3,4-dihydro-1H-pyrido[3,4-b]indole-2(9H)-carbothioamide,
(R)-1-(benzo[d][1,3]dioxol-5-yl)-N-(3,4-dichlorobenzyl)-3,4-dihydro-1H-pyrido[3,4-b]indole-2(9H)-carbothioamide,
(R)-1-(benzo[d][1,3]dioxol-5-yl)-N-(4-fluorobenzyl)-3,4-dihydro-1H-pyrido[3,4-b]indole-2(9H)-carbothioamide,
(R)-1-(benzo[d][1,3]dioxol-5-yl)-N-(3,4-dimethylbenzyl)-3,4-dihydro-1H-pyrido[3,4-b]indole-2(9H)-carbothioamide,
(R)-1-(benzo[d][1,3]dioxol-5-yl)-N-(3-chlorobenzyl)-3,4-dihydro-1H-pyrido[3,4-b]indole-2(9H)-carbothioamide,
(R)-1-(benzo[d][1,3]dioxol-5-yl)-N-(naphthalen-1-ylmethyl)-3,4-dihydro-1H-pyrido[3,4-b]indole-2(9H)-carbothioamide,
(3,4-difluorophenyl)-(1-phenyl-1,3,4,9-tetrahydro-β-carbolin-2-yl)-methanone,
6-methoxy-1,2,3,4-tetrahydronorharmane,
1,2,3,4-tetrahydronorharman-3-carboxylic acid,
6-methoxy-1,2,3,4-tetrahydronorharman-1-carboxylic acid,
1-(4-methoxyphenyl)-1,2,3,4-tetrahydronorharman-3-carboxylic acid,
1-methyl-1,2,3,4-tetrahydronorharman-3-carboxylic acid,
1-methyl-1,2,3,4-tetrahydronorharman-1,3-dicarboxylic acid,
1-(diethylmethyl)-1,2,3,4-tetrahydronorharman-3-carboxylic acid,
(6-bromo-1,2,3,4-tetrahydronorharman-1-yl)-3-propionic acid,
1-isobutyl-1,2,3,4-tetrahydronorharman-3-carboxylic acid,
1-phenyl-1,2,3,4-tetrahydronorharman-3-carboxylic acid,
1-propyl-1,2,3,4-tetrahydronorharman-3-carboxylic acid,
1-methyl-1-methoxycarbonyl-6-benzyloxy-1,2,3,4-tetrahydronorharmane,
1-methyl-1-methoxycarbonyl-6-methoxy-1,2,3,4-tetrahydronorharmane,
1-methyl-1-methoxycarbonyl-6-hydroxy-1,2,3,4-tetrahydronorharmane,
1-methyl-1-methoxycarbonyl-6-chloro-1,2,3,4-tetrahydronorharmane,
1-methyl-1-methoxycarbonyl-6-bromo-1,2,3,4-tetrahydronorharmane,
1-methyl-2-N-acetyl-6-methoxy-1,2,3,4-tetrahydro-β-carboline,
2-N-acetyl-1,2,3,4-tetrahydro-β-carboline,
1-methyl-2-N-acetyl-6-methoxy-1,2,3,4-tetrahydro-β-carboline,
4-chlorobenzyl (1S,3R)-1-(2,4-dichlorophenyl)-1,2,3,4-tetrahydro-β-carboline-3-carboxamide,
(3R)-1-(1-benzylindol-3-yl)-2-tert-butoxycarbonyl-1,2,3,4-tetrahydro-β-carboline-3-carboxylic acid,
(3R)-1-(1-butylindol-3-yl)-2-tert-butoxycarbonyl-1,2,3,4-tetrahydro-β-carboline-3-carboxylic acid,
(1S,3R)-1-(indol-3-yl)-2-tert-butoxycarbonyl-1,2,3,4-tetrahydro-β-carboline-3-carboxylic acid,
(1S,3R)-1-(1-methylindol-3-yl)-2-tert-butoxycarbonyl-1,2,3,4-tetrahydro-β-carboline-3-carboxylic acid,
benzothiazol-2-yl (1S,3R)-1-cyclohexyl-2-tert-butoxycarbonyl-1,2,3,4-tetrahydro-β-carboline-3-carboxylic acid,
benzothiazol-2-yl (1S,3R)-1-cyclohexyl-1,2,3,4-tetrahydro-β-carboline-3-carboxylic acid,
1-(4-chlorophenyl)-1,2,3,4-tetrahydro-β-carboline,
1-(4-bromophenyl)-1,2,3,4-tetrahydro-β-carboline,
1-(4-nitrophenyl)-1,2,3,4-tetrahydro-β-carboline,
1-(4-dimethylaminophenyl)-1,2,3,4-tetrahydro-β-carboline,
1-(4-diethylaminophenyl)-1,2,3,4-tetrahydro-β-carboline,
1-(2,4-dimethoxyphenyl)-1,2,3,4-tetrahydro-β-carboline,
1-(3,4-dimethoxyphenyl)-1,2,3,4-tetrahydro-β-carboline,
1-(2,5-dimethoxyphenyl)-1,2,3,4-tetrahydro-β-carboline,
1-(3,5-dimethoxyphenyl)-1,2,3,4-tetrahydro-β-carboline,
1-(3,4,5-trimethoxyphenyl)-1,2,3,4-tetrahydro-β-carboline,
1-(4-nitrobenzo[d][1,3]dioxol-5-yl)-1,2,3,4-tetrahydro-β-carboline,
1-(2-fluorenyl)-1,2,3,4-tetrahydro-β-carboline,
1-(9-ethyl-9H-carbazol-3-yl)-1,2,3,4-tetrahydro-β-carboline,
6-chloro-1-(4-methylphenyl)-2,3,4,9-tetrahydro-1H-β-carboline,
methyl 6-chloro-1-(4-methylphenyl)-1,3,4,9-tetrahydro-2H-β-carboline-2-carboxylate,
6-chloro-1-(4-methylphenyl)-2-(3-phenylpropanoyl)-2,3,4,9-tetrahydro-1H-β-carboline,
phenylmethyl 6-chloro-1-(4-methylphenyl)-1,3,4,9-tetrahydro-2H-β-carboline-2-carboxylate,
6-fluoro-1-(4-methylphenyl)-2,3,4,9-tetrahydro-1H-β-carboline,
methyl 6-fluoro-1-(4-methylphenyl)-1,3,4,9-tetrahydro-2H-β-carboline-2-carboxylate,
6-fluoro-1-(4-methylphenyl)-2-(3-phenylpropanoyl)-2,3,4,9-tetrahydro-1H-β-carboline,
phenylmethyl 6-fluoro-1-(4-methylphenyl)-1,3,4,9-tetrahydro-2H-β-carboline-2-carboxylate,
6-bromo-1-(4-methylphenyl)-2,3,4,9-tetrahydro-1H-β-carboline,
methyl 6-bromo-1-(4-methylphenyl)-1,3,4,9-tetrahydro-2H-β-carboline-2-carboxylate,
6-bromo-1-(4-methylphenyl)-2-(3-phenylpropanoyl)-2,3,4,9-tetrahydro-1H-β-carboline,
phenylmethyl 6-bromo-1-(4-methylphenyl)-1,3,4,9-tetrahydro-2H-β-carboline-2-carboxylate,
(1R)-6-chloro-1-(4-methylphenyl)-2-(3-phenylpropanoyl)-2,3,4,9-tetrahydro-1H-β-carboline,
(1S)-6-chloro-1-(4-methylphenyl)-2-(3-phenylpropanoyl)-2,3,4,9-tetrahydro-1H-β-carboline,
1-(4-methylphenyl)-2-(methylsulfonyl)-2,3,4,9-tetrahydro-1H-β-carboline,
2-acetyl-1-(4-methylphenyl)-2,3,4,9-tetrahydro-1H-β-carboline,
1-(4-methylphenyl)-2-(3-phenylpropanoyl)-2,3,4,9-tetrahydro-1H-β-carboline,
6-(methyloxy)-1-(4-methylphenyl)-2-(3-phenylpropanoyl)-2,3,4,9-tetrahydro-1H-β-carboline,
6-methyl-1-(4-methylphenyl)-2-(3-phenylpropanoyl)-2,3,4,9-tetrahydro-1H-β-carboline,
(1R/1S)-1-(2,3-dihydro-1-benzofuran-5-yl)-2,3,4,9-tetrahydro-1H-β-carboline, or
1-(1,3-benzodioxol-5-yl)-2-(2-pyrimidinyl)-2,3,4,9-tetrahydro-1H-β-carboline.

As will be evident to one of skill in the art, Compounds provided herein comprise at least one stereocenter, and may exist as a racemic mixture or as an enantiomerically pure composition. In one embodiment, a Compound provided herein is the (S) isomer, in an enantiomerically pure composition. In certain embodiments, the enantiomeric excess (e.e.) is about 90%, about 95%, about 99% or about 99.9% or greater.

In another embodiment, provided herein are Compounds having Formula (II):

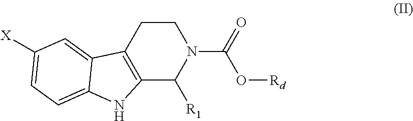

or a pharmaceutically acceptable salt, racemate, tautomer or stereoisomer thereof, wherein,
X is hydrogen; C₁ to C₈ alkyl optionally substituted with one or more halogen substituents; hydroxyl; halogen; thioether; sulfinyl, sulfonyl, cyano, or C₁ to C₈ alkoxy optionally substituted with aryl or C₁ to C₈ alkyl;
R₁ is hydroxyl; C₁ to C₈ alkyl optionally substituted with thioether, heteroaryl, or aryl optionally substituted with one or more independently selected Rₒ substituents; C₂ to C₈ alkenyl; C₂ to C₈ alkynyl; heterocyclyl optionally substituted with one or more substituents independently selected from halogen, oxo, amino, alkylamino, acetamino, thiol, or thioether; heteroaryl optionally substituted with one or more substituents independently selected from halogen, oxo, amino, alkylamino, acetamino, thiol, C₁ to C₈ alkoxy or thioether; or aryl optionally substituted with one or more independently selected Rₒ substituents;
Rₒ is a halogen; cyano; nitro; sulfonyl optionally substituted with C₁ to C₈ alkyl or heterocyclyl; amino optionally substituted with C₁ to C₆ alkyl, —C(O)—R_{b}, —C(O)O—R_{b}, sulfonyl, alkylsulfonyl or heterocyclyl optionally substituted with —C(O)O—Rₙ; —C(O)—NH—R_{b}; heterocyclyl; heteroaryl; C₁ to C₈ alkyl optionally substituted with one or more substituents independently selected from hydroxyl, halogen, amino or heterocyclyl, wherein amino and heterocyclyl are optionally substituted with one or more C₁ to C₈ alkyl substituents optionally substituted with one or more substituents independently selected from C₁ to C₈ alkoxy, amino, alkylamino, or heterocyclyl; —C(O)—Rₙ; or —ORₐ;
Rₐ is hydrogen; C₂ to C₈ alkenyl; C₂ to C₈ alkynyl; —C(O)—Rₙ; —C(O)O—R_{b}; —C(O)—NH—R_{b}; aryl; heteroaryl optionally substituted with halogen, amino, C₁ to C₈ alkyl optionally substituted with heterocyclyl or alkylsulfonyl, wherein heterocyclyl is optionally substituted with C₁ to C₈ alkyl; heterocyclyl optionally substituted with hydroxyl, C₁ to C₈ alkyl optionally substituted with hydroxyl, heterocyclyl, aryl, heteroaryl, —C(O)O—R_{b}, sulfonyl, C₁ to C₈ alkyl-sulfonyl or silyl-C₁ to C₈ alkyl-sulfonyl; C₁ to C₈ alkyl optionally substituted with one or more substituents independently selected from hydroxyl, halogen, cyano, C₁ to C₈ alkoxy, amino, monoalkylamino, dialkylamino, acetamino, sulfonyl, thioether, sulfonamide, —C(O)—R_{b}, —C(O)O—R_{b}, —OR_{g}, aryl, heterocyclyl, or heteroaryl, wherein the amino is optionally substituted with cycloalkyl, the alkyl of the monoalkylamino or dialkylamino is independently optionally substituted with one or more substituents independently selected from hydroxyl, C₁ to C₈ alkoxy, imino, amino, heterocyclyl, dialkylamino or heteroaryl optionally substituted with C₁ to C₈ alkyl, wherein the heteroaryl is optionally substituted with one or more substituents independently selected from —C(O)—NH—R_{b}, amino, cyano or heterocyclyl optionally substituted with one or more acetate or hydroxyl substituents, wherein the acetamino is optionally substituted with C₁ to C₈ alkyl, C₁ to C₈ alkoxy optionally substituted with C₁ to C₈ alkoxy, cycloalkyl, heteroaryl, sulfonyl, or alkylsulfonyl, wherein the heterocyclyl is optionally substituted with imino, —C(O)—Rₙ, —C(O)O—Rₙ, oxo or C₁ to C₈ alkyl optionally substituted with hydroxyl, further wherein the C₁ to C₈ alkoxy is optionally substituted with heterocyclyl, further wherein the sulfonamide is optionally substituted with C₁ to C₈ alkyl or cycloalkyl, further wherein the amino is optionally substituted with alkoxycarbonyl, alkylsulfonyl, cycloalkylsulfonyl, imidazole, isothiazole, pyrazole, pyridine, pyrazine, pyrimidine, pyrrole, thiazole, isoxazole, triazine or sulfonyl substituted with C₁ to C₈ alkyl, wherein pyridine, isoxazole, and thiazole are each optionally substituted with C₁ to C₈ alkyl;
R_{b} is hydroxyl; amino; or monoalkylamino or dialkylamino, wherein alkyl is independently optionally substituted with one or more substituents independently selected from hydroxyl, amino, alkylamino, C₁ to C₈ alkoxy, heterocyclyl optionally substituted with one or more substituents independently selected from C₁ to C₈ alkyl, oxo, —C(O)O—Rₙ, or heteroaryl optionally substituted with C₁ to C₈ alkyl; C₁ to C₈ alkoxy; C₂ to C₈ alkenyl; C₂ to C₈ alkynyl; aryl optionally substituted with one or more substituents independently selected from halogen or C₁ to C₈ alkoxy; heteroaryl; heterocyclyl optionally substituted with one or more substituents independently selected from acetamino, —C(O)O—Rₙ, heterocyclyl, or C₁ to C₈ alkyl optionally substituted with hydroxyl, C₁ to C₈ alkoxy, amino, monoalkylamino or dialkylamino; or C₁ to C₈ alkyl optionally substituted with one or more substituents independently selected from C₁ to C₈ alkoxy, aryl, amino, —C(O)O—Rₙ, or heterocyclyl, wherein the amino and heterocyclyl are optionally substituted with one or more substituents independently selected from C₁ to C₈ alkyl, oxo, or —C(O)O—Rₙ;
Rₙ is hydroxyl; C₁ to C₈ alkoxy; amino; or aryl optionally substituted with halogen or C₁ to C₈ alkyl; and
R_{g} is hydroxyl; amino optionally substituted with cycloalkyl or heteroaryl; heteroaryl optionally substituted with amino; or heterocyclyl, wherein the heterocyclyl is optionally substituted with —C(O)—Rₙ.

In another embodiment, provided herein are Compounds having Formula (III):

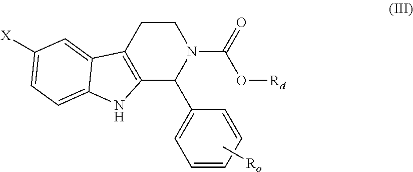

or a pharmaceutically acceptable salt, racemate, tautomer or stereoisomer thereof, wherein,
X is hydrogen; C₁ to C₈ alkyl optionally substituted with one or more halogen substituents; hydroxyl; halogen; thioether; sulfinyl, sulfonyl, cyano, or C₁ to C₈ alkoxy optionally substituted with aryl or C₁ to C₈ alkyl;
Rₒ is a halogen; cyano; nitro; sulfonyl optionally substituted with C₁ to C₈ alkyl or heterocyclyl; amino optionally substituted with C₁ to C₆ alkyl, —C(O)—R_{b}, —C(O)O—R_{b}, sulfonyl, alkylsulfonyl or heterocyclyl optionally substituted with —C(O)O—Rₙ; —C(O)—NH—R_{b}; heterocyclyl; heteroaryl; C₁ to C₈ alkyl optionally substituted with one or more substituents independently selected from hydroxyl, halogen, amino or heterocyclyl, wherein amino and heterocyclyl are optionally substituted with one or more C₁ to C₈ alkyl substituents optionally substituted with one or more substituents independently selected from C₁ to C₈ alkoxy, amino, alkylamino, or heterocyclyl; —C(O)—Rₙ; or —ORₐ;
Rₐ is hydrogen; C₂ to C₈ alkenyl; C₂ to C₈ alkynyl; —C(O)—Rₙ; —C(O)O—R_{b}; —C(O)—NH—R_{b}; aryl; heteroaryl optionally substituted with halogen, amino, C₁ to C₈ alkyl optionally substituted with heterocyclyl or alkylsulfonyl, wherein heterocyclyl is optionally substituted with C₁ to C₈ alkyl; heterocyclyl optionally substituted with hydroxyl, C₁ to C₈ alkyl optionally substituted with hydroxyl, heterocyclyl, aryl, heteroaryl, —C(O)O—R_{b}, sulfonyl, C₁ to C₈ alkyl-sulfonyl or silyl-C₁ to C₈ alkyl-sulfonyl; C₁ to C₈ alkyl optionally substituted with one or more substituents independently selected from hydroxyl, halogen, cyano, C₁ to C₈ alkoxy, amino, monoalkylamino, dialkylamino, acetamino, sulfonyl, thioether, sulfonamide, —C(O)—R_{b}, —C(O)O—R_{b}, —OR_{g}, aryl, heterocyclyl, or heteroaryl, wherein the amino is optionally substituted with cycloalkyl, the alkyl of the monoalkylamino or dialkylamino is independently optionally substituted with one or more substituents independently selected from hydroxyl, C₁ to C₈ alkoxy, imino, amino, heterocyclyl, dialkylamino or heteroaryl optionally substituted with C₁ to C₈ alkyl, wherein the heteroaryl is optionally substituted with one or more substituents independently selected from —C(O)—NH—R_{b}, amino, cyano or heterocyclyl optionally substituted with one or more acetate or hydroxyl substituents, wherein the acetamino is optionally substituted with C₁ to C₈ alkyl, C₁ to C₈ alkoxy optionally substituted with C₁ to C₈ alkoxy, cycloalkyl, heteroaryl, sulfonyl, or alkylsulfonyl, wherein the heterocyclyl is optionally substituted with imino, —C(O)—Rₙ, —C(O)O—Rₙ, oxo or C₁ to C₈ alkyl optionally substituted with hydroxyl, further wherein the C₁ to C₈ alkoxy is optionally substituted with heterocyclyl, further wherein the sulfonamide is optionally substituted with C₁ to C₈ alkyl or cycloalkyl, further wherein the amino is optionally substituted with alkoxycarbonyl, alkylsulfonyl, cycloalkylsulfonyl, imidazole, isothiazole, pyrazole, pyridine, pyrazine, pyrimidine, pyrrole, thiazole, isoxazole, triazine or sulfonyl substituted with C₁ to C₈ alkyl, wherein pyridine, isoxazole, and thiazole are each optionally substituted with C₁ to C₈ alkyl;
R_{b} is hydroxyl; amino; or monoalkylamino or dialkylamino, wherein alkyl is independently optionally substituted with one or more substituents independently selected from hydroxyl, amino, alkylamino, C₁ to C₈ alkoxy, heterocyclyl optionally substituted with one or more substituents independently selected from C₁ to C₈ alkyl, oxo, —C(O)O—Rₙ, or heteroaryl optionally substituted with C₁ to C₈ alkyl; C₁ to C₈ alkoxy; C₂ to C₈ alkenyl; C₂ to C₈ alkynyl; aryl optionally substituted with one or more substituents independently selected from halogen or C₁ to C₈ alkoxy; heteroaryl; heterocyclyl optionally substituted with one or more substituents independently selected from acetamino, —C(O)O—Rₙ, heterocyclyl, or C₁ to C₈ alkyl optionally substituted with hydroxyl, C₁ to C₈ alkoxy, amino, monoalkylamino or dialkylamino; or C₁ to C₈ alkyl optionally substituted with one or more substituents independently selected from C₁ to C₈ alkoxy, aryl, amino, —C(O)O—Rₙ, or heterocyclyl, wherein the amino and heterocyclyl are optionally substituted with one or more substituents independently selected from C₁ to C₈ alkyl, oxo, or —C(O)O—Rₙ;
Rₙ is hydroxyl; C₁ to C₈ alkoxy; amino; or aryl optionally substituted with halogen or C₁ to C₈ alkyl; and
R_{g} is hydroxyl; amino optionally substituted with cycloalkyl or heteroaryl; heteroaryl optionally substituted with amino; or heterocyclyl, wherein the heterocyclyl is optionally substituted with —C(O)—Rₙ.

In another embodiment, provided herein are Compounds having Formula (III):

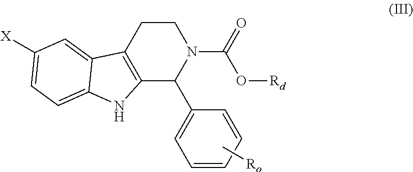

or a pharmaceutically acceptable salt, racemate, tautomer or stereoisomer thereof, wherein,
X is halogen;
Rₒ is halogen, substituted or unsubstituted C₁ to C₈ alkyl or ORₐ;
Rₐ is H, C₁ to C₈ alkyl optionally substituted with one or more substituents independently selected from hydroxyl and halogen; and
R_{d} is phenyl optionally substituted with one or more alkoxy or halogen substituents.

In one embodiment, X is chloro or bromo.

In one embodiment, R_{d} is chloro or bromo.

In one embodiment, Rₒ is ORₐ.

In one embodiment, Rₐ is methyl, ethyl, propyl, isopropyl, butyl, or pentyl, each optionally substituted with one or more hydroxyl substituents.

In another embodiment, provided herein are Compounds having Formula (III):

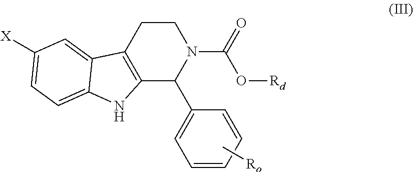

or a pharmaceutically acceptable salt, racemate, tautomer or stereoisomer thereof, wherein,
X is halogen;
Rₒ is halogen, substituted or unsubstituted C₁ to C₈ alkyl or ORₐ;
Rₐ is H, or C₁ to C₈ alkyl optionally substituted with one or more substituents independently selected from hydroxyl and halogen; and
R_{d} is phenyl optionally substituted with one or more halogen substituents.

In another embodiment, provided herein are Compounds having Formula (IV):

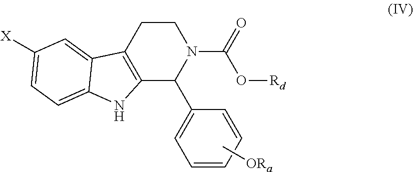

or a pharmaceutically acceptable salt, racemate, tautomer or stereoisomer thereof, wherein,
X is halogen;
Rₐ is H, C₁ to C₈ alkyl optionally substituted with one or more substituents independently selected from hydroxyl and halogen; and
R_{d} is phenyl substituted with one or more halogen substituents.

In another embodiment, provided herein are Compounds having Formula (V):

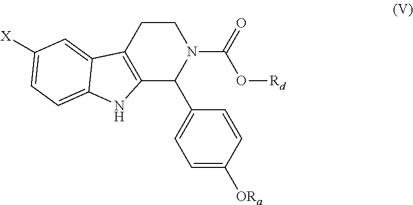

or a pharmaceutically acceptable salt, racemate, tautomer or stereoisomer thereof, wherein,
X is halogen;
Rₐ is H, C₁ to C₈ alkyl optionally substituted with one or more substituents independently selected from hydroxyl and halogen; and
R_{d} is phenyl substituted with one or more halogen substituents.

In another embodiment, provided herein are Compounds having Formula (V):

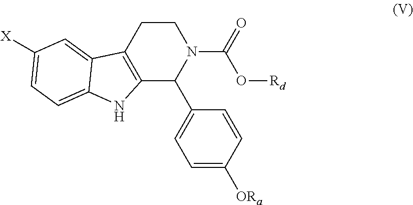

or a pharmaceutically acceptable salt, racemate, tautomer or stereoisomer thereof, wherein,
X is halogen;
Rₐ is H, C₁ to C₈ alkyl optionally substituted with one or more substituents independently selected from hydroxyl and halogen; and
R_{d} is phenyl substituted on a para position with a halogen substituent.

In another embodiment, provided herein are Compounds having Formula (VI):

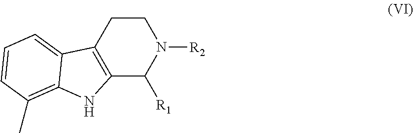

or a pharmaceutically acceptable salt, racemate, tautomer or stereoisomer thereof, wherein,
R₁ is hydroxyl; C₁ to C₈ alkyl optionally substituted with thioether, heteroaryl, or aryl optionally substituted with one or more independently selected Rₒ substituents; C₂ to C₈ alkenyl; C₂ to C₈ alkynyl; heterocyclyl optionally substituted with one or more substituents independently selected from halogen, oxo, amino, alkylamino, acetamino, thiol, or thioether; heteroaryl optionally substituted with one or more substituents independently selected from halogen, oxo, amino, alkylamino, acetamino, thiol, C₁ to C₈ alkoxy or thioether; or aryl optionally substituted with one or more independently selected Rₒ substituents;
R₂ is hydrogen; hydroxyl; halogen; heteroaryl optionally substituted with hydroxyl, C₁ to C₈ alkyl optionally substituted with halogen, aryl or —C(O)—Rₑ, aryl optionally substituted with halogen or C₁ to C₈ alkoxy, heteroaryl, —C(O)O—R_{d}, —C(O)—N(R_{d}R_{d}); C₁ to C₈ alkyl optionally substituted with hydroxyl, C₁ to C₈ alkoxy, heterocyclyl, heteroaryl, or aryl; —C(O)—R_{c}; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; amino optionally substituted with one or more substituents independently selected from C₁ to C₈ alkyl or aryl; aryl optionally substituted with one or more substituents independently selected from halogen, haloalkyl, hydroxyl, C₁ to C₈ alkoxy, or C₁ to C₈ alkyl; —C(O)—Rₙ; heterocyclyl optionally substituted with —C(O)—Rₙ; heteroaryl; thiazoleamino; C₁ to C₈ alkyl optionally substituted with one or more substituents independently selected from halogen, C₁ to C₈ alkoxy, phenyloxy, aryl, —C(O)—Rₙ, —O—C(O)—Rₙ, hydroxyl, or amino optionally substituted with —C(O)O—Rₙ or —C(O)—Rₙ; cycloalkyl; or C₂ to C₈ alkenyl optionally substituted with aryl;
R_{d} is at each occurrence independently hydrogen; C₂ to C₈ alkenyl; C₂ to C₈ alkynyl; aryl optionally substituted with one or more substituents independently selected from halogen, nitro, C₁ to C₈ alkyl, —C(O)O—Rₑ, or —ORₑ; cycloalkyl; or C₁ to C₈ alkyl optionally substituted with one or more substituents independently selected from halogen, C₁ to C₈ alkyl, C₁ to C₈ alkoxy, cycloalkyl, phenyloxy, aryl, heteroaryl, —C(O)—Rₙ, —C(O)O—Rₙ, or hydroxyl, wherein the aryl is optionally substituted with one or more substituents independently selected from C₁ to C₈ alkyl, C₁ to C₈ alkoxy, cyano, halogen or haloalkyl;
Rₑ is hydrogen; C₁ to C₈ alkyl optionally substituted with one or more substituents independently selected from halogen, —C(O)—Rₙ or C₁ to C₈ alkoxy; cycloalkyl optionally substituted with oxo; or aryl optionally substituted with one or more substituents independently selected from halogen or C₁ to C₈ alkoxy;
R_{f} is C₁ to C₈ alkyl optionally substituted with one or more substituents independently selected from halogen, hydroxyl, C₁ to C₈ alkoxy, cyano, aryl, or —C(O)—Rₙ, wherein the C₁ to C₈ alkoxy is optionally substituted with one or more C₁ to C₈ alkoxy substituents and the aryl is optionally substituted with one or more substituents independently selected from halogen, hydroxyl, C₁ to C₈ alkoxy, cyano, or C₁ to C₈ alkyl; and
Rₙ is hydroxyl; C₁ to C₈ alkoxy; amino; or aryl optionally substituted with halogen or C₁ to C₈ alkyl.

In another embodiment, provided herein are Compounds having Formula (VII):

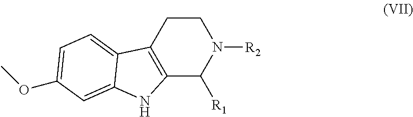

or a pharmaceutically acceptable salt, racemate, tautomer or stereoisomer thereof, wherein,
R₁ is hydroxyl; C₁ to C₈ alkyl optionally substituted with thioether, heteroaryl, or aryl optionally substituted with one or more independently selected Rₒ substituents; C₂ to C₈ alkenyl; C₂ to C₈ alkynyl; heterocyclyl optionally substituted with one or more substituents independently selected from halogen, oxo, amino, alkylamino, acetamino, thiol, or thioether; heteroaryl optionally substituted with one or more substituents independently selected from halogen, oxo, amino, alkylamino, acetamino, thiol, C₁ to C₈ alkoxy or thioether; or aryl optionally substituted with one or more independently selected Rₒ substituents;
R₂ is hydrogen; hydroxyl; halogen; heteroaryl optionally substituted with hydroxyl, C₁ to C₈ alkyl optionally substituted with halogen, aryl or —C(O)—R_{c}, aryl optionally substituted with halogen or C₁ to C₈ alkoxy, heteroaryl, —C(O)O—R_{d}, —C(O)—N(R_{d}R_{d}); C₁ to C₈ alkyl optionally substituted with hydroxyl, C₁ to C₈ alkoxy, heterocyclyl, heteroaryl, or aryl; —C(O)—R_{c}; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; amino optionally substituted with one or more substituents independently selected from C₁ to C₈ alkyl or aryl; aryl optionally substituted with one or more substituents independently selected from halogen, haloalkyl, hydroxyl, C₁ to C₈ alkoxy, or C₁ to C₈ alkyl; —C(O)—Rₙ; heterocyclyl optionally substituted with —C(O)—Rₙ; heteroaryl; thiazoleamino; C₁ to C₈ alkyl optionally substituted with one or more substituents independently selected from halogen, C₁ to C₈ alkoxy, phenyloxy, aryl, —C(O)—Rₙ, —O—C(O)—Rₙ, hydroxyl, or amino optionally substituted with —C(O)O—Rₙ or —C(O)—Rₙ; cycloalkyl; or C₂ to C₈ alkenyl optionally substituted with aryl;
R_{d} is at each occurrence independently hydrogen; C₂ to C₈ alkenyl; C₂ to C₈ alkynyl; aryl optionally substituted with one or more substituents independently selected from halogen, nitro, C₁ to C₈ alkyl, —C(O)O—Rₑ, or —ORₑ; cycloalkyl; or C₁ to C₈ alkyl optionally substituted with one or more substituents independently selected from halogen, C₁ to C₈ alkyl, C₁ to C₈ alkoxy, cycloalkyl, phenyloxy, aryl, heteroaryl, —C(O)—Rₙ, —C(O)O—Rₙ, or hydroxyl, wherein the aryl is optionally substituted with one or more substituents independently selected from C₁ to C₈ alkyl, C₁ to C₈ alkoxy, cyano, halogen or haloalkyl;
Rₑ is hydrogen; C₁ to C₈ alkyl optionally substituted with one or more substituents independently selected from halogen, —C(O)—Rₙ or C₁ to C₈ alkoxy; cycloalkyl optionally substituted with oxo; or aryl optionally substituted with one or more substituents independently selected from halogen or C₁ to C₈ alkoxy;
R_{f} is C₁ to C₈ alkyl optionally substituted with one or more substituents independently selected from halogen, hydroxyl, C₁ to C₈ alkoxy, cyano, aryl, or —C(O)—Rₙ, wherein the C₁ to C₈ alkoxy is optionally substituted with one or more C₁ to C₈ alkoxy substituents and the aryl is optionally substituted with one or more substituents independently selected from halogen, hydroxyl, C₁ to C₈ alkoxy, cyano, or C₁ to C₈ alkyl; and
Rₙ is hydroxyl; C₁ to C₈ alkoxy; amino; or aryl optionally substituted with halogen or C₁ to C₈ alkyl.

In another embodiment, the Compounds set forth above have a formula selected from Formula (Ia), Formula (Ha), Formula (Ma), Formula (IVa), Formula (Va), Formula (VIa), Formula (VIIa), Formula (VIIIa), Formula (IXa), Formula (Xa), Formula (XIa), Formula (XIIa), Formula (XIIIa) and Formula (XIVa):

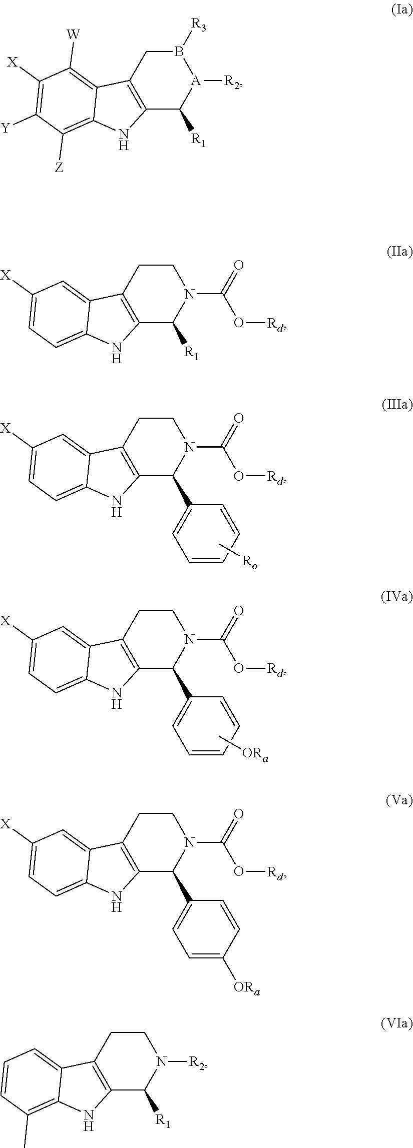

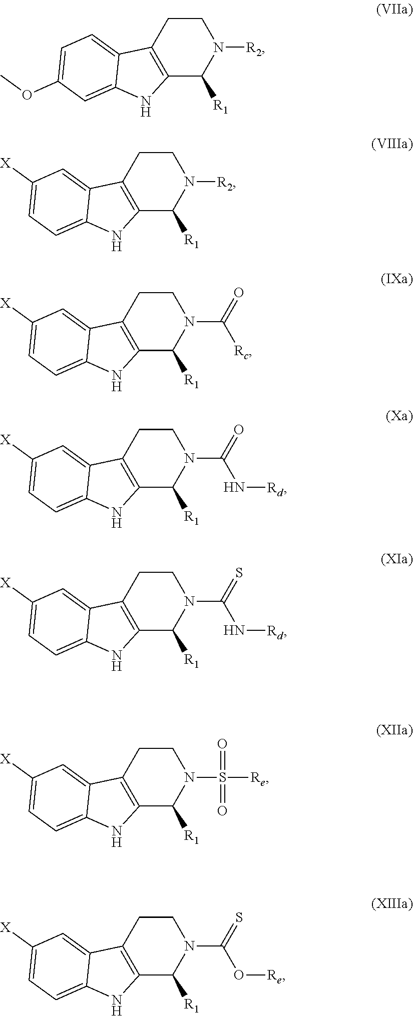

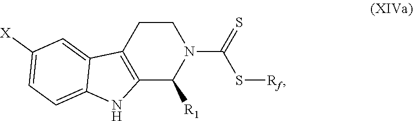

Illustrative examples of Compounds or a pharmaceutically acceptable salt, racemate, tautomer or stereoisomer thereof provided herein include:

TABLE 1

| | |
|---|---|
| 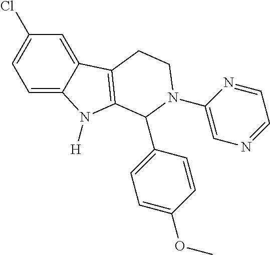 [SMILES: COc1ccc(C2c3[nH]c4ccc(Cl)cc4c3CCN2c2cnccn2)cc1] | 1 |
| 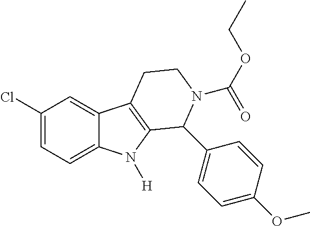 [SMILES: CCOC(=O)N1CCc2c([nH]c3ccc(Cl)cc23)C1c1ccc(OC)cc1] | 2 |
| 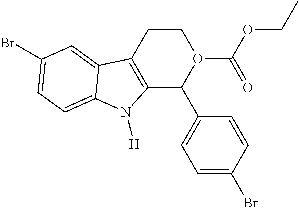 | 3 |
| 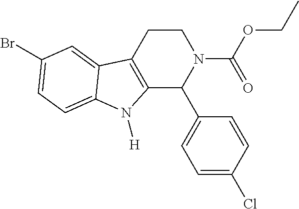 [SMILES: CCOC(=O)N1CCc2c([nH]c3ccc(Br)cc23)C1c1ccc(Cl)cc1] | 4 |
| 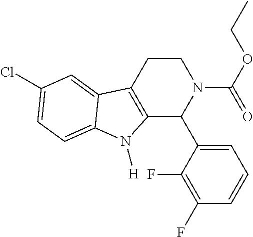 [SMILES: CCOC(=O)N1CCc2c([nH]c3ccc(Cl)cc23)C1c1cccc(F)c1F] | 5 |
| 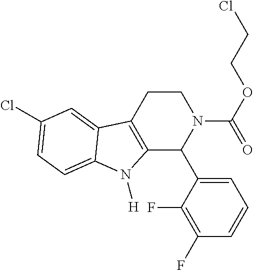 [SMILES: O=C(OCCCl)N1CCc2c([nH]c3ccc(Cl)cc23)C1c1cccc(F)c1F] | 6 |
| 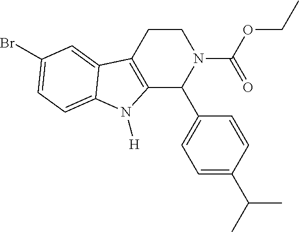 [SMILES: CCOC(=O)N1CCc2c([nH]c3ccc(Br)cc23)C1c1ccc(C(C)C)cc1] | 7 |
| 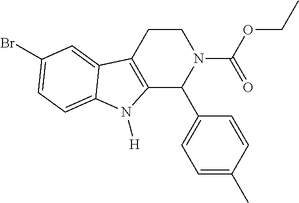 [SMILES: CCOC(=O)N1CCc2c([nH]c3ccc(Br)cc23)C1c1ccc(C)cc1] | 8 |
| 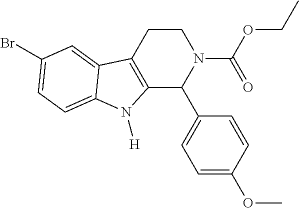 [SMILES: CCOC(=O)N1CCc2c([nH]c3ccc(Br)cc23)C1c1ccc(OC)cc1] | 9 |
| 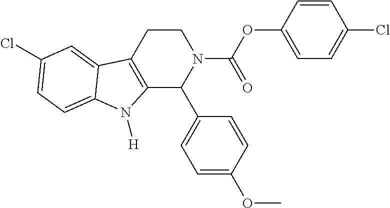 [SMILES: COc1ccc(C2c3[nH]c4ccc(Cl)cc4c3CCN2C(=O)Oc2ccc(Cl)cc2)cc1] | 10 |
| 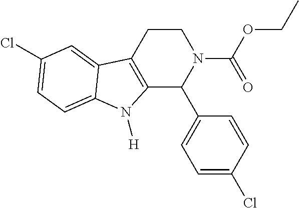 [SMILES: CCOC(=O)N1CCc2c([nH]c3ccc(Cl)cc23)C1c1ccc(Cl)cc1] | 11 |
| 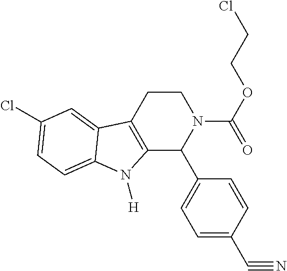 [SMILES: N#Cc1ccc(C2c3[nH]c4ccc(Cl)cc4c3CCN2C(=O)OCCCl)cc1] | 12 |
| 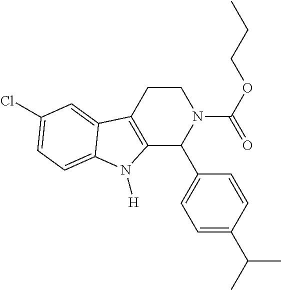 [SMILES: CCCOC(=O)N1CCc2c([nH]c3ccc(Cl)cc23)C1c1ccc(C(C)C)cc1] | 13 |
| 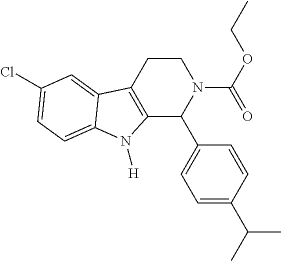 [SMILES: CCOC(=O)N1CCc2c([nH]c3ccc(Cl)cc23)C1c1ccc(C(C)C)cc1] | 14 |
| 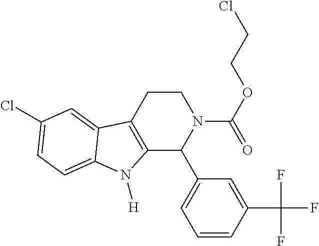 [SMILES: O=C(OCCCl)N1CCc2c([nH]c3ccc(Cl)cc23)C1c1cccc(C(F)(F)F)c1] | 15 |
| 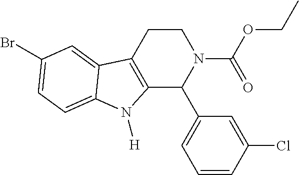 [SMILES: CCOC(=O)N1CCc2c([nH]c3ccc(Br)cc23)C1c1cccc(Cl)c1] | 16 |
| 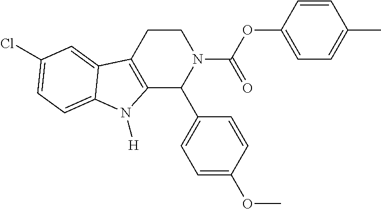 [SMILES: COc1ccc(C2c3[nH]c4ccc(Cl)cc4c3CCN2C(=O)Oc2ccc(C)cc2)cc1] | 17 |
| 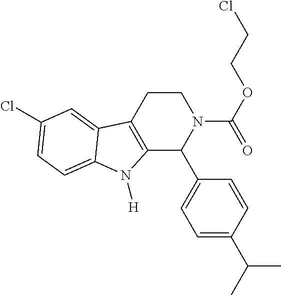 [SMILES: CC(C)c1ccc(C2c3[nH]c4ccc(Cl)cc4c3CCN2C(=O)OCCCl)cc1] | 18 |
| 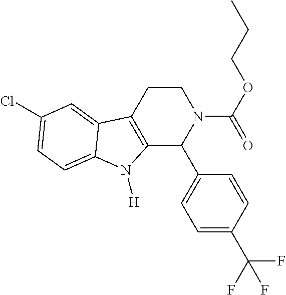 [SMILES: CCCOC(=O)N1CCc2c([nH]c3ccc(Cl)cc23)C1c1ccc(C(F)(F)F)cc1] | 19 |
| 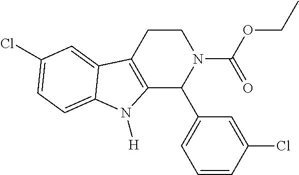 [SMILES: CCOC(=O)N1CCc2c([nH]c3ccc(Cl)cc23)C1c1cccc(Cl)c1] | 20 |
| 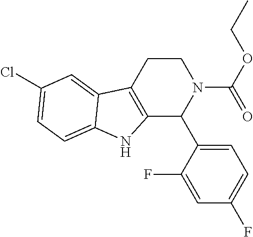 [SMILES: CCOC(=O)N1CCc2c([nH]c3ccc(Cl)cc23)C1c1ccc(F)cc1F] | 21 |
| 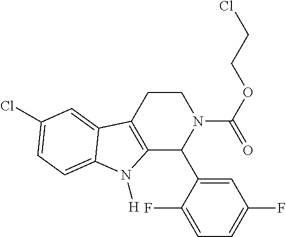 [SMILES: O=C(OCCCl)N1CCc2c([nH]c3ccc(Cl)cc23)C1c1cc(F)ccc1F] | 22 |
| 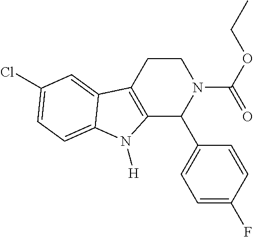 [SMILES: CCOC(=O)N1CCc2c([nH]c3ccc(Cl)cc23)C1c1ccc(F)cc1] | 23 |
| 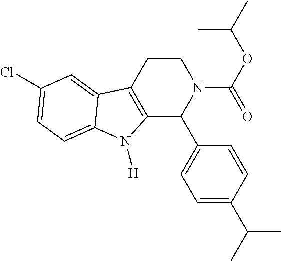 [SMILES: CC(C)OC(=O)N1CCc2c([nH]c3ccc(Cl)cc23)C1c1ccc(C(C)C)cc1] | 24 |
| 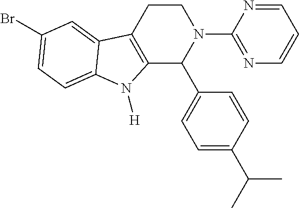 [SMILES: CC(C)c1ccc(C2c3[nH]c4ccc(Br)cc4c3CCN2c2ncccn2)cc1] | 25 |
| 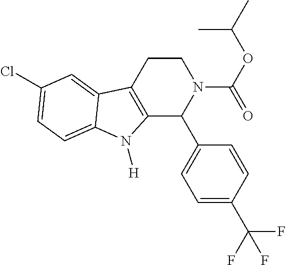 [SMILES: CC(C)OC(=O)N1CCc2c([nH]c3ccc(Cl)cc23)C1c1ccc(C(F)(F)F)cc1] | 26 |
| 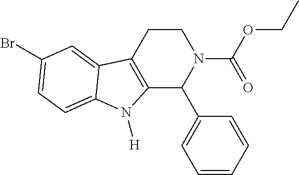 [SMILES: CCOC(=O)N1CCc2c([nH]c3ccc(Br)cc23)C1c1ccccc1] | 27 |
| 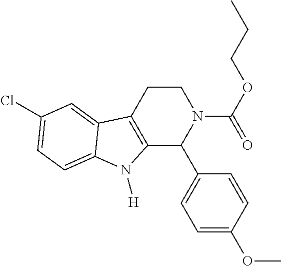 [SMILES: CCCOC(=O)N1CCc2c([nH]c3ccc(Cl)cc23)C1c1ccc(OC)cc1] | 28 |
| 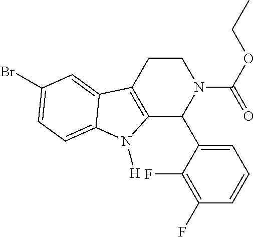 [SMILES: CCOC(=O)N1CCc2c([nH]c3ccc(Br)cc23)C1c1cccc(F)c1F] | 29 |
| 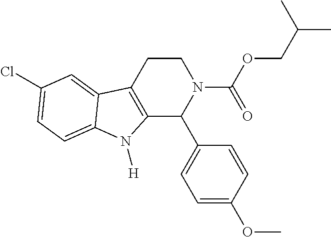 [SMILES: COc1ccc(C2c3[nH]c4ccc(Cl)cc4c3CCN2C(=O)OCC(C)C)cc1] | 30 |
| 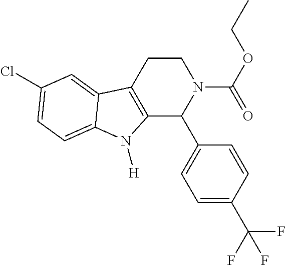 [SMILES: CCOC(=O)N1CCc2c([nH]c3ccc(Cl)cc23)C1c1ccc(C(F)(F)F)cc1] | 31 |
| 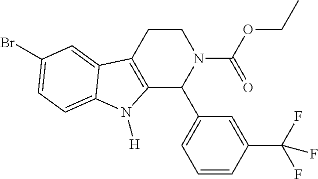 [SMILES: CCOC(=O)N1CCc2c([nH]c3ccc(Br)cc23)C1c1cccc(C(F)(F)F)c1] | 32 |
| 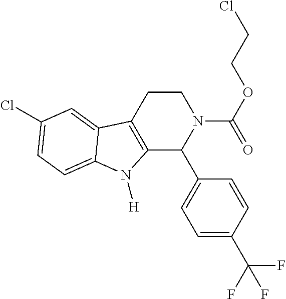 [SMILES: O=C(OCCCl)N1CCc2c([nH]c3ccc(Cl)cc23)C1c1ccc(C(F)(F)F)cc1] | 33 |
| 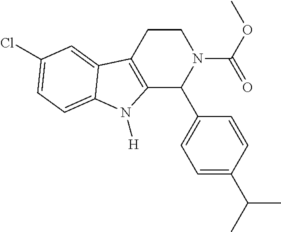 [SMILES: COC(=O)N1CCc2c([nH]c3ccc(Cl)cc23)C1c1ccc(C(C)C)cc1] | 34 |
| 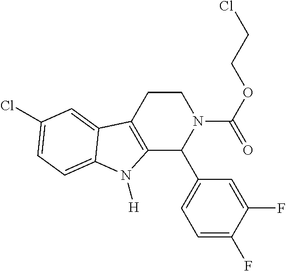 [SMILES: O=C(OCCCl)N1CCc2c([nH]c3ccc(Cl)cc23)C1c1ccc(F)c(F)c1] | 35 |
| 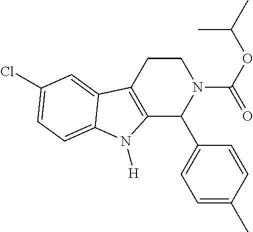 [SMILES: Cc1ccc(C2c3[nH]c4ccc(Cl)cc4c3CCN2C(=O)OC(C)C)cc1] | 36 |
| 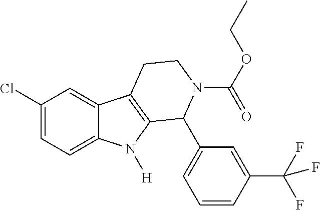 [SMILES: CCOC(=O)N1CCc2c([nH]c3ccc(Cl)cc23)C1c1cccc(C(F)(F)F)c1] | 37 |
| 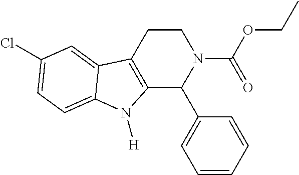 [SMILES: CCOC(=O)N1CCc2c([nH]c3ccc(Cl)cc23)C1c1ccccc1] | 38 |
| 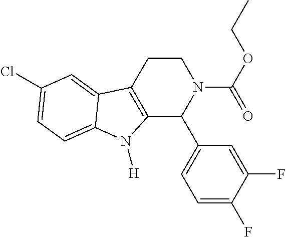 [SMILES: CCOC(=O)N1CCc2c([nH]c3ccc(Cl)cc23)C1c1ccc(F)c(F)c1] | 39 |
| 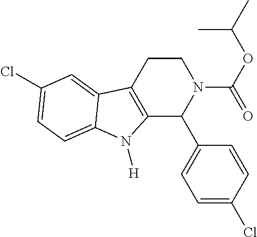 [SMILES: CC(C)OC(=O)N1CCc2c([nH]c3ccc(Cl)cc23)C1c1ccc(Cl)cc1] | 40 |
| 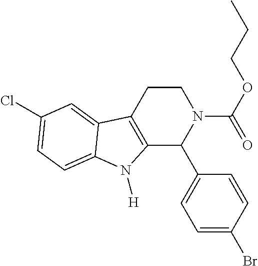 [SMILES: CCCOC(=O)N1CCc2c([nH]c3ccc(Cl)cc23)C1c1ccc(Br)cc1] | 41 |
| 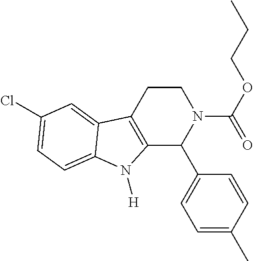 [SMILES: CCCOC(=O)N1CCc2c([nH]c3ccc(Cl)cc23)C1c1ccc(C)cc1] | 42 |
| 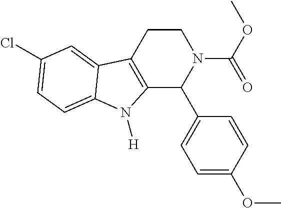 [SMILES: COC(=O)N1CCc2c([nH]c3ccc(Cl)cc23)C1c1ccc(OC)cc1] | 43 |
| 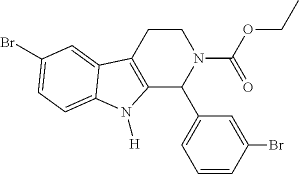 [SMILES: CCOC(=O)N1CCc2c([nH]c3ccc(Br)cc23)C1c1cccc(Br)c1] | 44 |
| 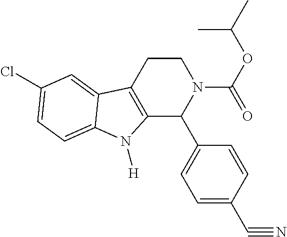 [SMILES: CC(C)OC(=O)N1CCc2c([nH]c3ccc(Cl)cc23)C1c1ccc(C#N)cc1] | 45 |
| 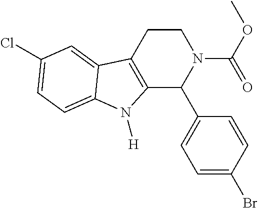 [SMILES: COC(=O)N1CCc2c([nH]c3ccc(Cl)cc23)C1c1ccc(Br)cc1] | 46 |
| 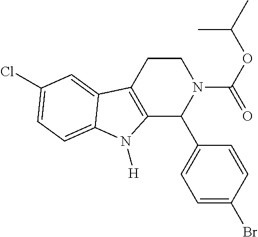 [SMILES: CC(C)OC(=O)N1CCc2c([nH]c3ccc(Cl)cc23)C1c1ccc(Br)cc1] | 47 |
| 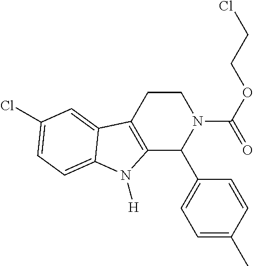 [SMILES: Cc1ccc(C2c3[nH]c4ccc(Cl)cc4c3CCN2C(=O)OCCCl)cc1] | 48 |
| 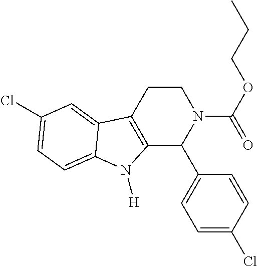 [SMILES: CCCOC(=O)N1CCc2c([nH]c3ccc(Cl)cc23)C1c1ccc(Cl)cc1] | 49 |
| 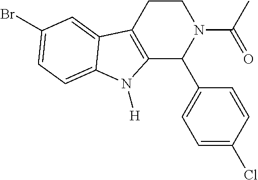 [SMILES: CC(=O)N1CCc2c([nH]c3ccc(Br)cc23)C1c1ccc(Cl)cc1] | 50 |
| 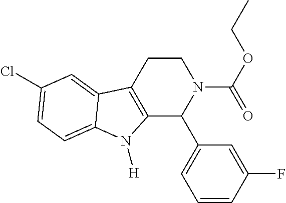 [SMILES: CCOC(=O)N1CCc2c([nH]c3ccc(Cl)cc23)C1c1cccc(F)c1] | 51 |
| 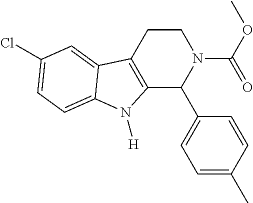 [SMILES: COC(=O)N1CCc2c([nH]c3ccc(Cl)cc23)C1c1ccc(C)cc1] | 52 |
| 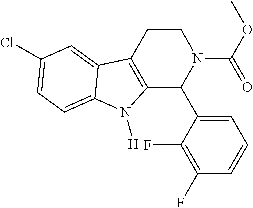 [SMILES: COC(=O)N1CCc2c([nH]c3ccc(Cl)cc23)C1c1cccc(F)c1F] | 53 |
| 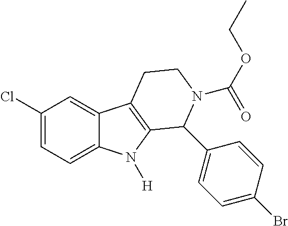 [SMILES: CCOC(=O)N1CCc2c([nH]c3ccc(Cl)cc23)C1c1ccc(Br)cc1] | 54 |
| 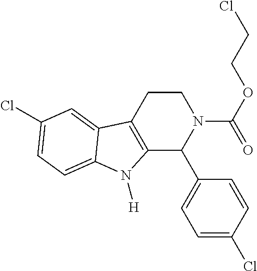 [SMILES: O=C(OCCCl)N1CCc2c([nH]c3ccc(Cl)cc23)C1c1ccc(Cl)cc1] | 55 |
| 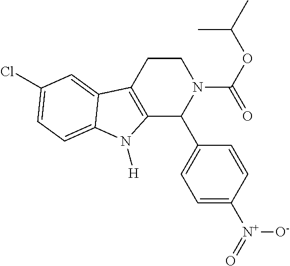 [SMILES: CC(C)OC(=O)N1CCc2c([nH]c3ccc(Cl)cc23)C1c1ccc([N+](=O)[O-])cc1] | 56 |
| 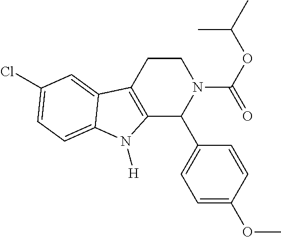 [SMILES: COc1ccc(C2c3[nH]c4ccc(Cl)cc4c3CCN2C(=O)OC(C)C)cc1] | 57 |
| 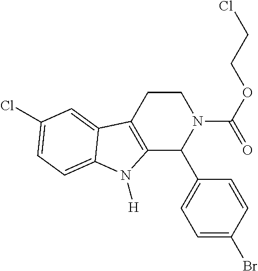 [SMILES: O=C(OCCCl)N1CCc2c([nH]c3ccc(Cl)cc23)C1c1ccc(Br)cc1] | 58 |
| 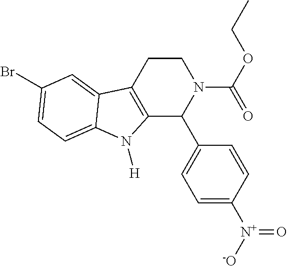 | 59 |
| 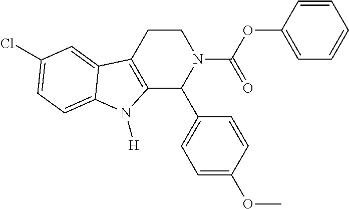 [SMILES: COc1ccc(C2c3[nH]c4ccc(Cl)cc4c3CCN2C(=O)Oc2ccccc2)cc1] | 60 |
| 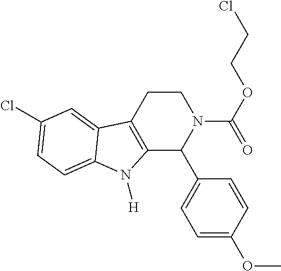 [SMILES: COc1ccc(C2c3[nH]c4ccc(Cl)cc4c3CCN2C(=O)OCCCl)cc1] | 61 |
| 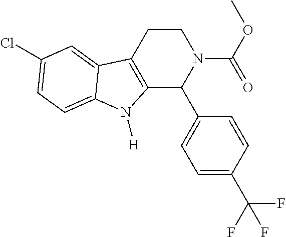 [SMILES: COC(=O)N1CCc2c([nH]c3ccc(Cl)cc23)C1c1ccc(C(F)(F)F)cc1] | 62 |
| 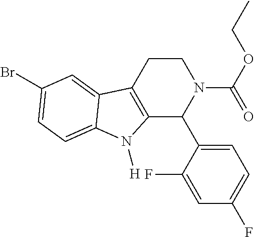 [SMILES: CCOC(=O)N1CCc2c([nH]c3ccc(Br)cc23)C1c1ccc(F)cc1F] | 63 |
| 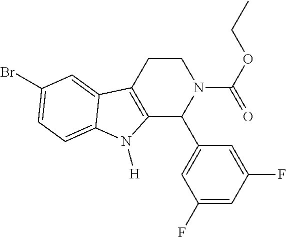 [SMILES: CCOC(=O)N1CCc2c([nH]c3ccc(Br)cc23)C1c1cc(F)cc(F)c1] | 64 |
| 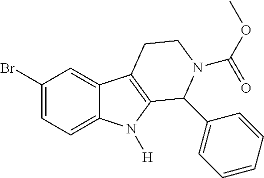 [SMILES: COC(=O)N1CCc2c([nH]c3ccc(Br)cc23)C1c1ccccc1] | 65 |
| 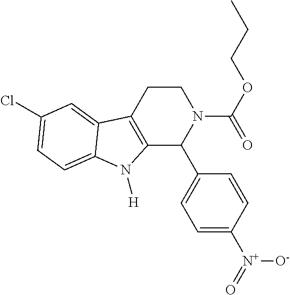 [SMILES: CCCOC(=O)N1CCc2c([nH]c3ccc(Cl)cc23)C1c1ccc([N+](=O)[O-])cc1] | 66 |
| 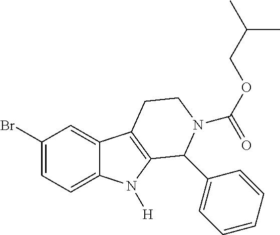 [SMILES: CC(C)COC(=O)N1CCc2c([nH]c3ccc(Br)cc23)C1c1ccccc1] | 67 |
| 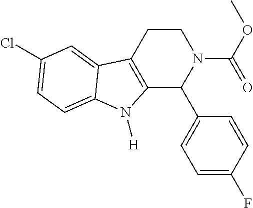 [SMILES: COC(=O)N1CCc2c([nH]c3ccc(Cl)cc23)C1c1ccc(F)cc1] | 68 |
| 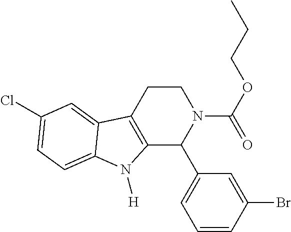 [SMILES: CCCOC(=O)N1CCc2c([nH]c3ccc(Cl)cc23)C1c1cccc(Br)c1] | 69 |
| 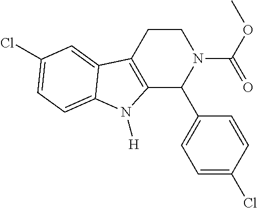 [SMILES: COC(=O)N1CCc2c([nH]c3ccc(Cl)cc23)C1c1ccc(Cl)cc1] | 70 |
| 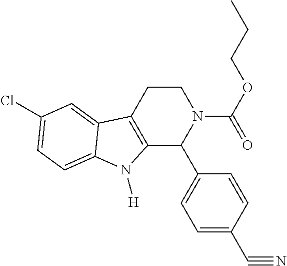 [SMILES: CCCOC(=O)N1CCc2c([nH]c3ccc(Cl)cc23)C1c1ccc(C#N)cc1] | 71 |
| 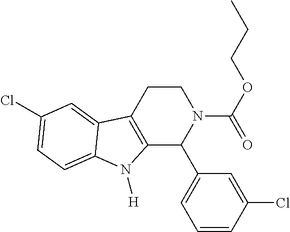 [SMILES: CCCOC(=O)N1CCc2c([nH]c3ccc(Cl)cc23)C1c1cccc(Cl)c1] | 72 |
| 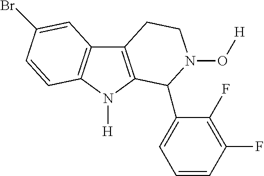 [SMILES: ON1CCc2c([nH]c3ccc(Br)cc23)C1c1cccc(F)c1F] | 73 |
| 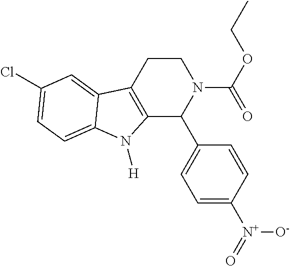 [SMILES: CCOC(=O)N1CCc2c([nH]c3ccc(Cl)cc23)C1c1ccc([N+](=O)[O-])cc1] | 74 |
| 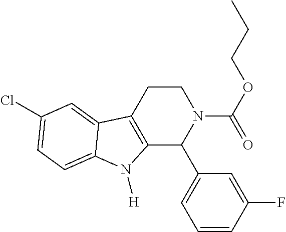 [SMILES: CCCOC(=O)N1CCc2c([nH]c3ccc(Cl)cc23)C1c1cccc(F)c1] | 75 |
| 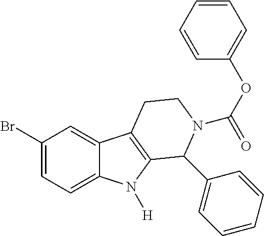 [SMILES: O=C(Oc1ccccc1)N1CCc2c([nH]c3ccc(Br)cc23)C1c1ccccc1] | 76 |
| 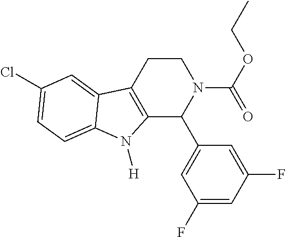 [SMILES: CCOC(=O)N1CCc2c([nH]c3ccc(Cl)cc23)C1c1cc(F)cc(F)c1] | 77 |
| 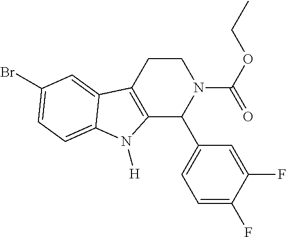 [SMILES: CCOC(=O)N1CCc2c([nH]c3ccc(Br)cc23)C1c1ccc(F)c(F)c1] | 78 |
| 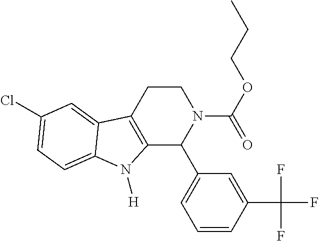 [SMILES: CCCOC(=O)N1CCc2c([nH]c3ccc(Cl)cc23)C1c1cccc(C(F)(F)F)c1] | 79 |
| 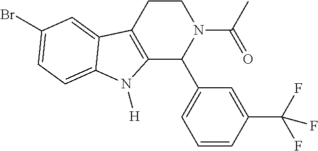 [SMILES: CC(=O)N1CCc2c([nH]c3ccc(Br)cc23)C1c1cccc(C(F)(F)F)c1] | 80 |
| 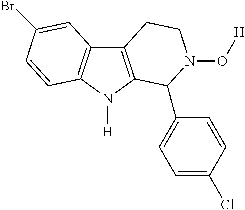 [SMILES: ON1CCc2c([nH]c3ccc(Br)cc23)C1c1ccc(Cl)cc1] | 81 |
| 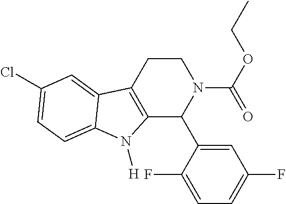 [SMILES: CCOC(=O)N1CCc2c([nH]c3ccc(Cl)cc23)C1c1cc(F)ccc1F] | 82 |
| 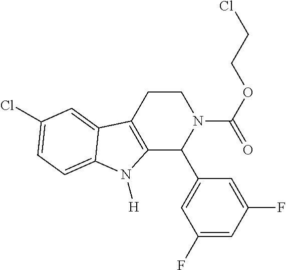 [SMILES: O=C(OCCCl)N1CCc2c([nH]c3ccc(Cl)cc23)C1c1cc(F)cc(F)c1] | 83 |
| 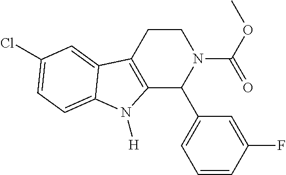 [SMILES: COC(=O)N1CCc2c([nH]c3ccc(Cl)cc23)C1c1cccc(F)c1] | 84 |
| 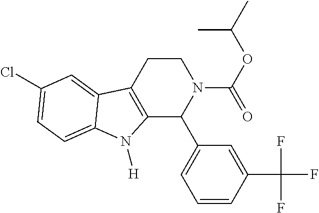 [SMILES: CC(C)OC(=O)N1CCc2c([nH]c3ccc(Cl)cc23)C1c1cccc(C(F)(F)F)c1] | 85 |
| 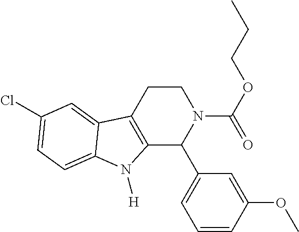 [SMILES: CCCOC(=O)N1CCc2c([nH]c3ccc(Cl)cc23)C1c1cccc(OC)c1] | 86 |
| 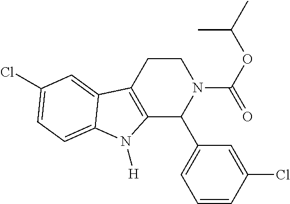 [SMILES: CC(C)OC(=O)N1CCc2c([nH]c3ccc(Cl)cc23)C1c1cccc(Cl)c1] | 87 |
| 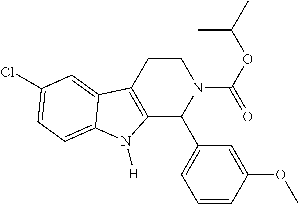 [SMILES: COc1cccc(C2c3[nH]c4ccc(Cl)cc4c3CCN2C(=O)OC(C)C)c1] | 88 |
| 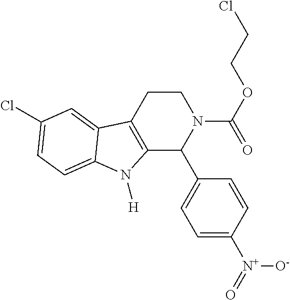 [SMILES: O=C(OCCCl)N1CCc2c([nH]c3ccc(Cl)cc23)C1c1ccc([N+](=O)[O-])cc1] | 89 |
| 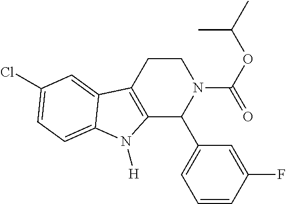 [SMILES: CC(C)OC(=O)N1CCc2c([nH]c3ccc(Cl)cc23)C1c1cccc(F)c1] | 90 |
| 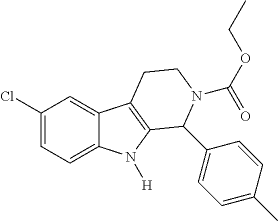 [SMILES: CCOC(=O)N1CCc2c([nH]c3ccc(Cl)cc23)C1c1ccc(C)cc1] | 91 |
| 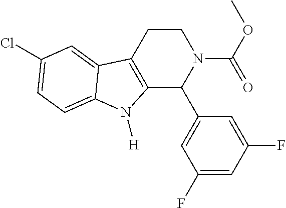 [SMILES: COC(=O)N1CCc2c([nH]c3ccc(Cl)cc23)C1c1cc(F)cc(F)c1] | 92 |
| 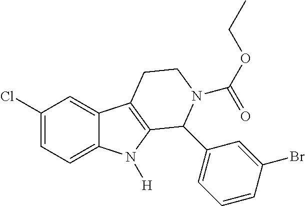 [SMILES: CCOC(=O)N1CCc2c([nH]c3ccc(Cl)cc23)C1c1cccc(Br)c1] | 93 |
| 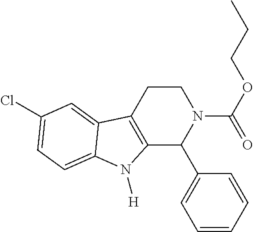 [SMILES: CCCOC(=O)N1CCc2c([nH]c3ccc(Cl)cc23)C1c1ccccc1] | 94 |
| 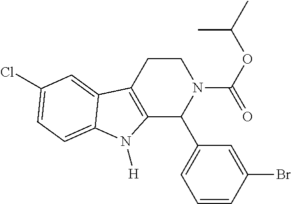 [SMILES: CC(C)OC(=O)N1CCc2c([nH]c3ccc(Cl)cc23)C1c1cccc(Br)c1] | 95 |
| 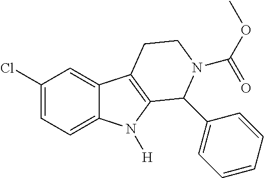 [SMILES: COC(=O)N1CCc2c([nH]c3ccc(Cl)cc23)C1c1ccccc1] | 96 |
| 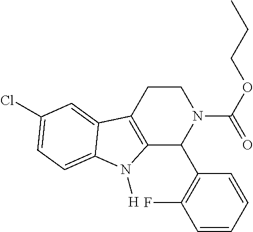 [SMILES: CCCOC(=O)N1CCc2c([nH]c3ccc(Cl)cc23)C1c1ccccc1F] | 97 |
| 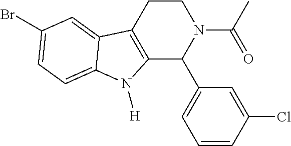 [SMILES: CC(=O)N1CCc2c([nH]c3ccc(Br)cc23)C1c1cccc(Cl)c1] | 98 |
| 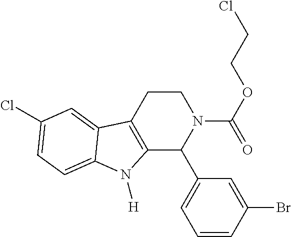 [SMILES: O=C(OCCCl)N1CCc2c([nH]c3ccc(Cl)cc23)C1c1cccc(Br)c1] | 99 |
| 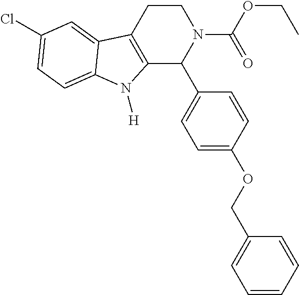 [SMILES: CCOC(=O)N1CCc2c([nH]c3ccc(Cl)cc23)C1c1ccc(OCc2ccccc2)cc1] | 100 |
| 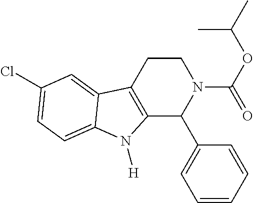 [SMILES: CC(C)OC(=O)N1CCc2c([nH]c3ccc(Cl)cc23)C1c1ccccc1] | 101 |
| 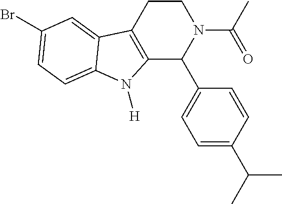 [SMILES: CC(=O)N1CCc2c([nH]c3ccc(Br)cc23)C1c1ccc(C(C)C)cc1] | 102 |
| 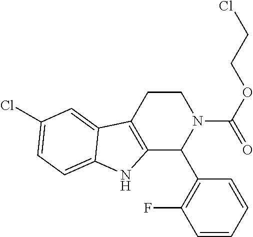 [SMILES: O=C(OCCCl)N1CCc2c([nH]c3ccc(Cl)cc23)C1c1ccccc1F] | 103 |
| 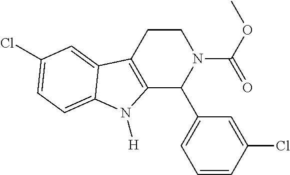 [SMILES: COC(=O)N1CCc2c([nH]c3ccc(Cl)cc23)C1c1cccc(Cl)c1] | 104 |
| 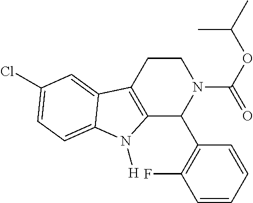 [SMILES: CC(C)OC(=O)N1CCc2c([nH]c3ccc(Cl)cc23)C1c1ccccc1F] | 105 |
| 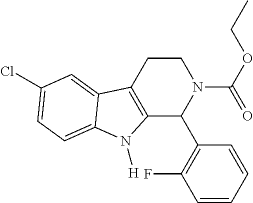 [SMILES: CCOC(=O)N1CCc2c([nH]c3ccc(Cl)cc23)C1c1ccccc1F] | 106 |
| 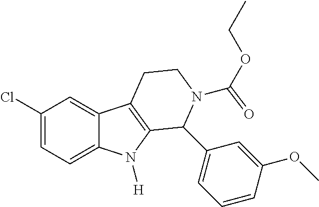 [SMILES: CCOC(=O)N1CCc2c([nH]c3ccc(Cl)cc23)C1c1cccc(OC)c1] | 107 |
| 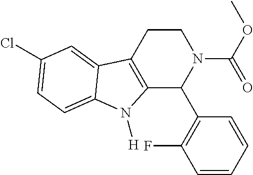 [SMILES: COC(=O)N1CCc2c([nH]c3ccc(Cl)cc23)C1c1ccccc1F] | 108 |
| 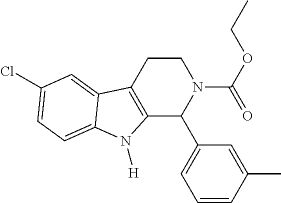 [SMILES: CCOC(=O)N1CCc2c([nH]c3ccc(Cl)cc23)C1c1cccc(C)c1] | 109 |
| 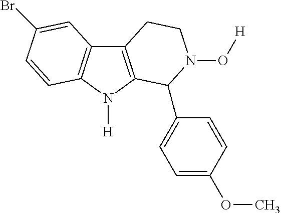 [SMILES: COc1ccc(C2c3[nH]c4ccc(Br)cc4c3CCN2O)cc1] | 110 |
| 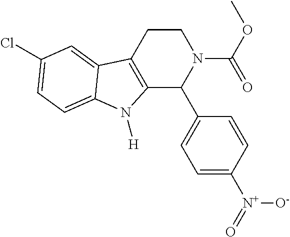 [SMILES: COC(=O)N1CCc2c([nH]c3ccc(Cl)cc23)C1c1ccc([N+](=O)[O-])cc1] | 111 |
| 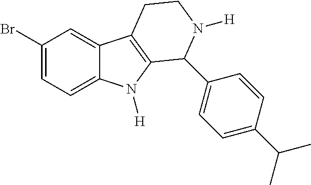 [SMILES: CC(C)c1ccc(C2NCCc3c2[nH]c2ccc(Br)cc32)cc1] | 112 |
| 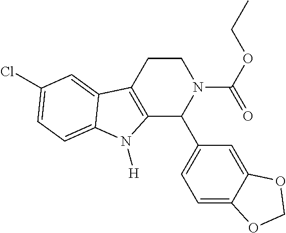 [SMILES: CCOC(=O)N1CCc2c([nH]c3ccc(Cl)cc23)C1c1ccc2c(c1)OCO2] | 113 |
| 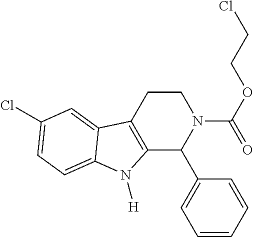 [SMILES: O=C(OCCCl)N1CCc2c([nH]c3ccc(Cl)cc23)C1c1ccccc1] | 114 |
| 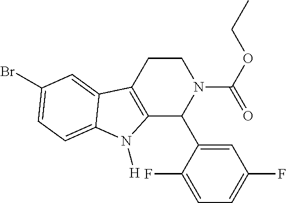 [SMILES: CCOC(=O)N1CCc2c([nH]c3ccc(Br)cc23)C1c1cc(F)ccc1F] | 115 |
| 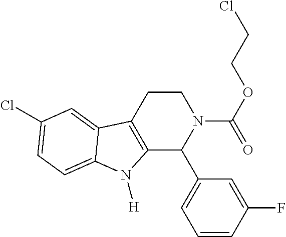 [SMILES: O=C(OCCCl)N1CCc2c([nH]c3ccc(Cl)cc23)C1c1cccc(F)c1] | 116 |
| 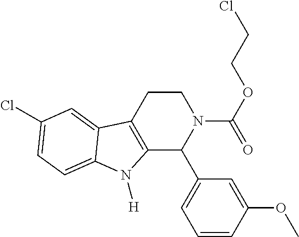 [SMILES: COc1cccc(C2c3[nH]c4ccc(Cl)cc4c3CCN2C(=O)OCCCl)c1] | 117 |
| 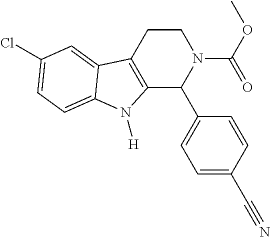 [SMILES: COC(=O)N1CCc2c([nH]c3ccc(Cl)cc23)C1c1ccc(C#N)cc1] | 118 |
| 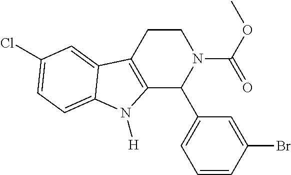 [SMILES: COC(=O)N1CCc2c([nH]c3ccc(Cl)cc23)C1c1cccc(Br)c1] | 119 |
| 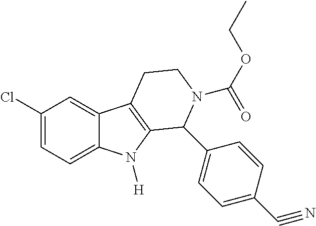 [SMILES: CCOC(=O)N1CCc2c([nH]c3ccc(Cl)cc23)C1c1ccc(C#N)cc1] | 120 |
| 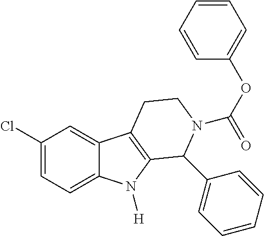 [SMILES: O=C(Oc1ccccc1)N1CCc2c([nH]c3ccc(Cl)cc23)C1c1ccccc1] | 121 |
| 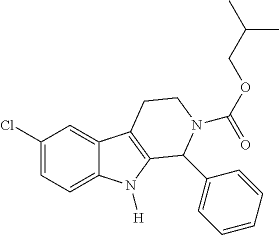 [SMILES: CC(C)COC(=O)N1CCc2c([nH]c3ccc(Cl)cc23)C1c1ccccc1] | 122 |
| 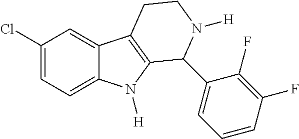 [SMILES: Fc1cccc(C2NCCc3c2[nH]c2ccc(Cl)cc32)c1F] | 123 |
| 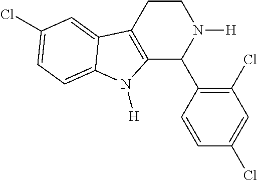 [SMILES: Clc1ccc(C2NCCc3c2[nH]c2ccc(Cl)cc32)c(Cl)c1] | 124 |
| 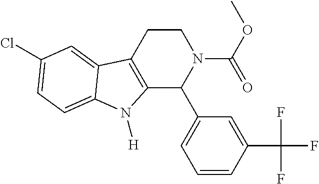 [SMILES: COC(=O)N1CCc2c([nH]c3ccc(Cl)cc23)C1c1cccc(C(F)(F)F)c1] | 125 |
| 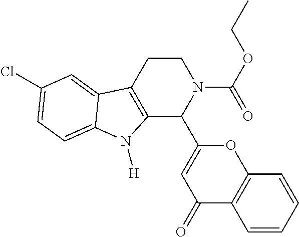 [SMILES: CCOC(=O)N1CCc2c([nH]c3ccc(Cl)cc23)C1c1cc(=O)c2ccccc2o1] | 126 |
| 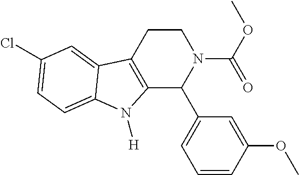 [SMILES: COC(=O)N1CCc2c([nH]c3ccc(Cl)cc23)C1c1cccc(OC)c1] | 127 |
| 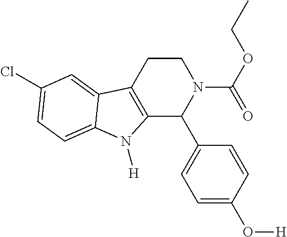 [SMILES: CCOC(=O)N1CCc2c([nH]c3ccc(Cl)cc23)C1c1ccc(O)cc1] | 128 |
| 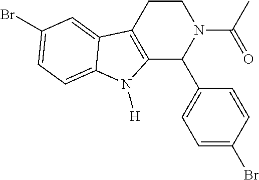 [SMILES: CC(=O)N1CCc2c([nH]c3ccc(Br)cc23)C1c1ccc(Br)cc1] | 129 |
| 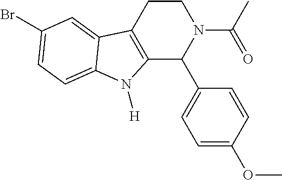 [SMILES: COc1ccc(C2c3[nH]c4ccc(Br)cc4c3CCN2C(C)=O)cc1] | 130 |
| 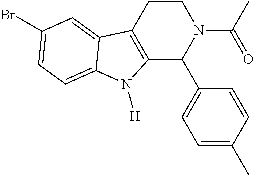 [SMILES: CC(=O)N1CCc2c([nH]c3ccc(Br)cc23)C1c1ccc(C)cc1] | 131 |
| 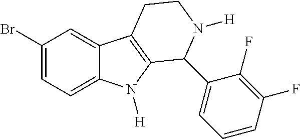 [SMILES: Fc1cccc(C2NCCc3c2[nH]c2ccc(Br)cc32)c1F] | 132 |
| 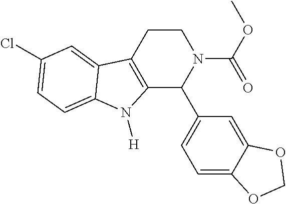 [SMILES: COC(=O)N1CCc2c([nH]c3ccc(Cl)cc23)C1c1ccc2c(c1)OCO2] | 133 |
| 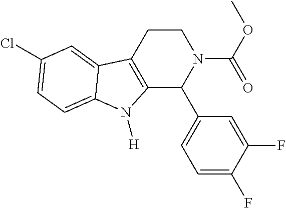 [SMILES: COC(=O)N1CCc2c([nH]c3ccc(Cl)cc23)C1c1ccc(F)c(F)c1] | 134 |
| 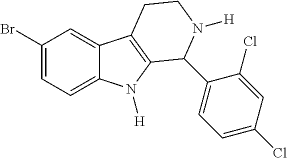 [SMILES: Clc1ccc(C2NCCc3c2[nH]c2ccc(Br)cc32)c(Cl)c1] | 135 |
| 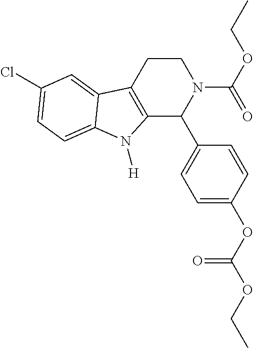 [SMILES: CCOC(=O)Oc1ccc(C2c3[nH]c4ccc(Cl)cc4c3CCN2C(=O)OCC)cc1] | 136 |
| 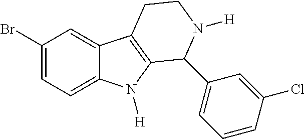 [SMILES: Clc1cccc(C2NCCc3c2[nH]c2ccc(Br)cc32)c1] | 137 |
| 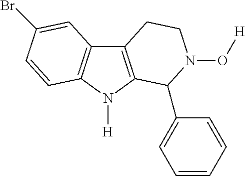 [SMILES: ON1CCc2c([nH]c3ccc(Br)cc23)C1c1ccccc1] | 138 |
| 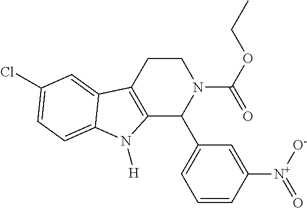 [SMILES: CCOC(=O)N1CCc2c([nH]c3ccc(Cl)cc23)C1c1cccc([N+](=O)[O-])c1] | 139 |
| 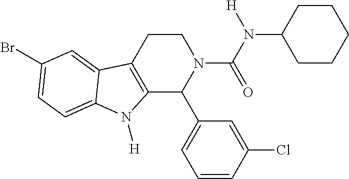 [SMILES: O=C(NC1CCCCC1)N1CCc2c([nH]c3ccc(Br)cc23)C1c1cccc(Cl)c1] | 140 |
| 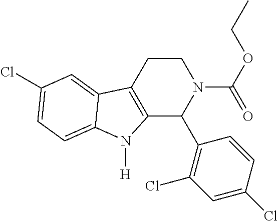 [SMILES: CCOC(=O)N1CCc2c([nH]c3ccc(Cl)cc23)C1c1ccc(Cl)cc1Cl] | 141 |
| 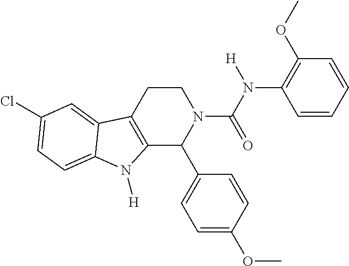 [SMILES: COc1ccc(C2c3[nH]c4ccc(Cl)cc4c3CCN2C(=O)Nc2ccccc2OC)cc1] | 142 |
| 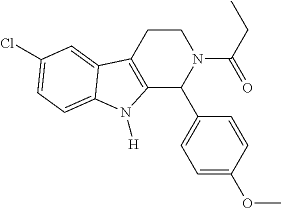 [SMILES: CCC(=O)N1CCc2c([nH]c3ccc(Cl)cc23)C1c1ccc(OC)cc1] | 143 |
| 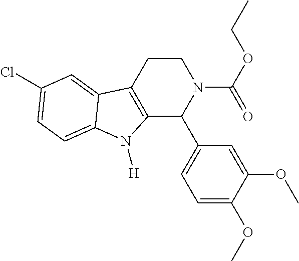 [SMILES: CCOC(=O)N1CCc2c([nH]c3ccc(Cl)cc23)C1c1ccc(OC)c(OC)c1] | 144 |
| 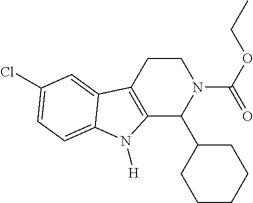 [SMILES: CCOC(=O)N1CCc2c([nH]c3ccc(Cl)cc23)C1C1CCCCC1] | 145 |
| 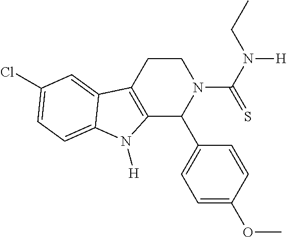 [SMILES: CCNC(=S)N1CCc2c([nH]c3ccc(Cl)cc23)C1c1ccc(OC)cc1] | 146 |
| 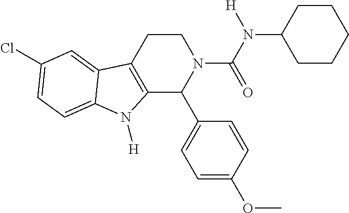 [SMILES: COc1ccc(C2c3[nH]c4ccc(Cl)cc4c3CCN2C(=O)NC2CCCCC2)cc1] | 147 |
| 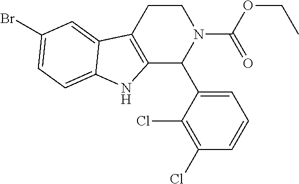 [SMILES: CCOC(=O)N1CCc2c([nH]c3ccc(Br)cc23)C1c1cccc(Cl)c1Cl] | 148 |
| 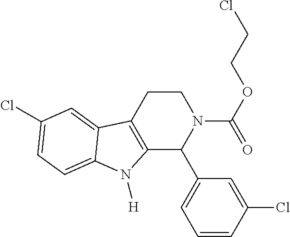 [SMILES: O=C(OCCCl)N1CCc2c([nH]c3ccc(Cl)cc23)C1c1cccc(Cl)c1] | 149 |
| 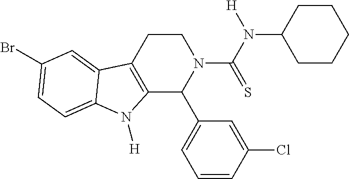 [SMILES: S=C(NC1CCCCC1)N1CCc2c([nH]c3ccc(Br)cc23)C1c1cccc(Cl)c1] | 150 |
| 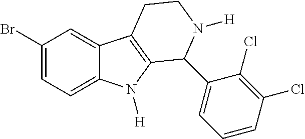 [SMILES: Clc1cccc(C2NCCc3c2[nH]c2ccc(Br)cc32)c1Cl] | 151 |
| 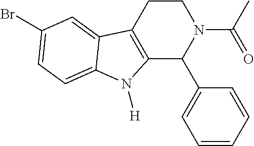 [SMILES: CC(=O)N1CCc2c([nH]c3ccc(Br)cc23)C1c1ccccc1] | 152 |
| 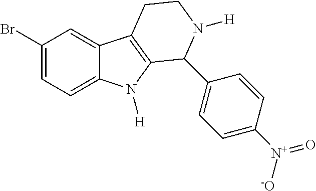 | 153 |
| 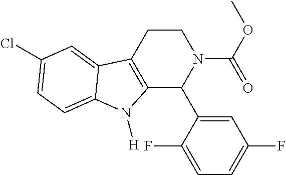 [SMILES: COC(=O)N1CCc2c([nH]c3ccc(Cl)cc23)C1c1cc(F)ccc1F] | 154 |
| 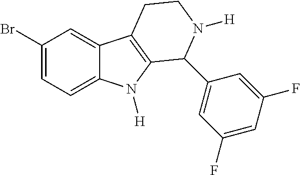 [SMILES: Fc1cc(F)cc(C2NCCc3c2[nH]c2ccc(Br)cc32)c1] | 155 |
| 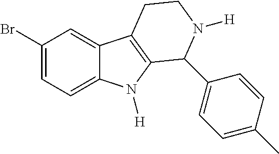 [SMILES: Cc1ccc(C2NCCc3c2[nH]c2ccc(Br)cc32)cc1] | 156 |
| 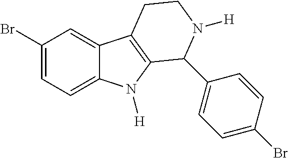 [SMILES: Brc1ccc(C2NCCc3c2[nH]c2ccc(Br)cc32)cc1] | 157 |
| 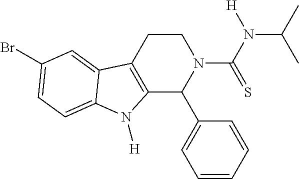 [SMILES: CC(C)NC(=S)N1CCc2c([nH]c3ccc(Br)cc23)C1c1ccccc1] | 158 |
| 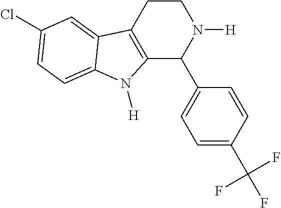 [SMILES: FC(F)(F)c1ccc(C2NCCc3c2[nH]c2ccc(Cl)cc32)cc1] | 159 |
| 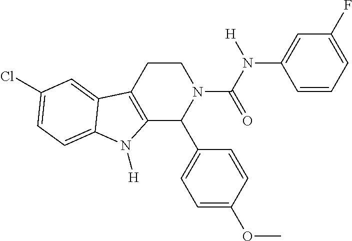 [SMILES: COc1ccc(C2c3[nH]c4ccc(Cl)cc4c3CCN2C(=O)Nc2cccc(F)c2)cc1] | 160 |
| 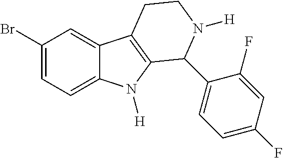 [SMILES: Fc1ccc(C2NCCc3c2[nH]c2ccc(Br)cc32)c(F)c1] | 161 |
| 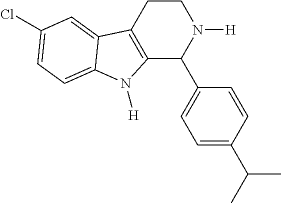 [SMILES: CC(C)c1ccc(C2NCCc3c2[nH]c2ccc(Cl)cc32)cc1] | 162 |
| 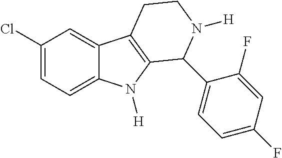 [SMILES: Fc1ccc(C2NCCc3c2[nH]c2ccc(Cl)cc32)c(F)c1] | 163 |
| 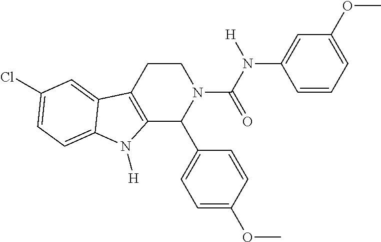 [SMILES: COc1ccc(C2c3[nH]c4ccc(Cl)cc4c3CCN2C(=O)Nc2cccc(OC)c2)cc1] | 164 |
| 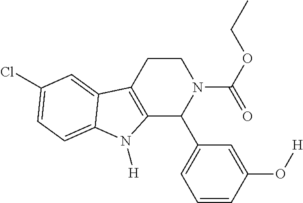 [SMILES: CCOC(=O)N1CCc2c([nH]c3ccc(Cl)cc23)C1c1cccc(O)c1] | 165 |
| 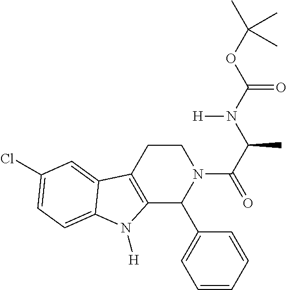 [SMILES: C[C@H](NC(=O)OC(C)(C)C)C(=O)N1CCc2c([nH]c3ccc(Cl)cc23)C1c1ccccc1] | 166 |
| 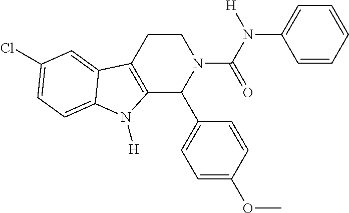 [SMILES: COc1ccc(C2c3[nH]c4ccc(Cl)cc4c3CCN2C(=O)Nc2ccccc2)cc1] | 167 |
| 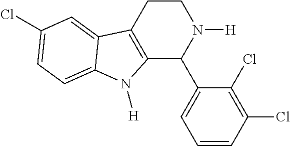 [SMILES: Clc1ccc2[nH]c3c(c2c1)CCNC3c1cccc(Cl)c1Cl] | 168 |
| 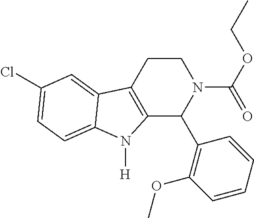 [SMILES: CCOC(=O)N1CCc2c([nH]c3ccc(Cl)cc23)C1c1ccccc1OC] | 169 |
| 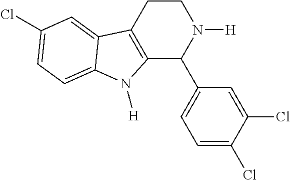 [SMILES: Clc1ccc2[nH]c3c(c2c1)CCNC3c1ccc(Cl)c(Cl)c1] | 170 |
| 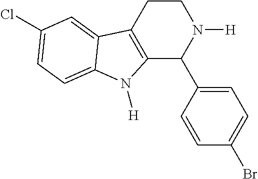 [SMILES: Clc1ccc2[nH]c3c(c2c1)CCNC3c1ccc(Br)cc1] | 171 |
| 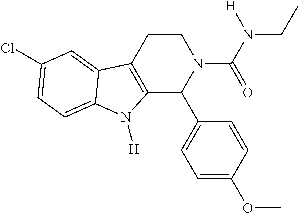 [SMILES: CCNC(=O)N1CCc2c([nH]c3ccc(Cl)cc23)C1c1ccc(OC)cc1] | 172 |
| 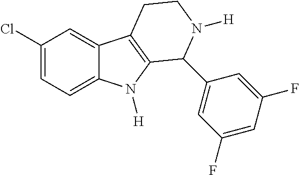 [SMILES: Fc1cc(F)cc(C2NCCc3c2[nH]c2ccc(Cl)cc32)c1] | 173 |
| 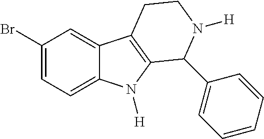 [SMILES: Brc1ccc2[nH]c3c(c2c1)CCNC3c1ccccc1] | 174 |
| 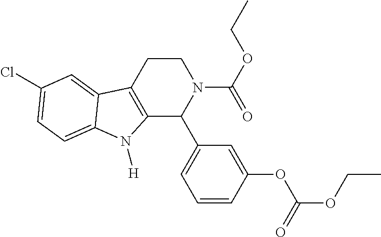 [SMILES: CCOC(=O)Oc1cccc(C2c3[nH]c4ccc(Cl)cc4c3CCN2C(=O)OCC)c1] | 175 |
| 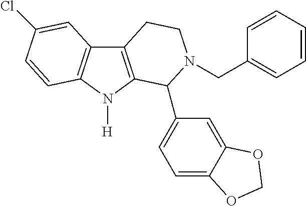 [SMILES: Clc1ccc2[nH]c3c(c2c1)CCN(Cc1ccccc1)C3c1ccc2c(c1)OCO2] | 176 |
| 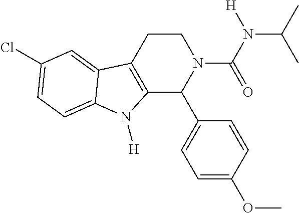 [SMILES: COc1ccc(C2c3[nH]c4ccc(Cl)cc4c3CCN2C(=O)NC(C)C)cc1] | 177 |
| 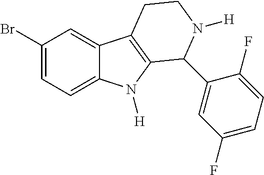 [SMILES: Fc1ccc(F)c(C2NCCc3c2[nH]c2ccc(Br)cc32)c1] | 178 |
| 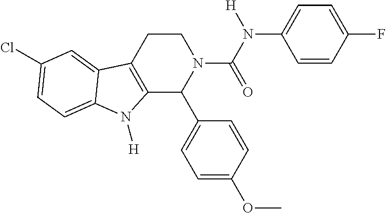 [SMILES: COc1ccc(C2c3[nH]c4ccc(Cl)cc4c3CCN2C(=O)Nc2ccc(F)cc2)cc1] | 179 |
| 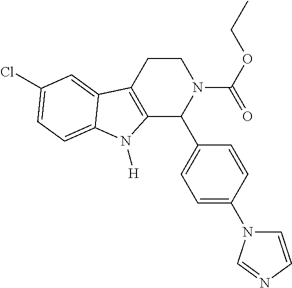 [SMILES: CCOC(=O)N1CCc2c([nH]c3ccc(Cl)cc23)C1c1ccc(-n2ccnc2)cc1] | 180 |
| 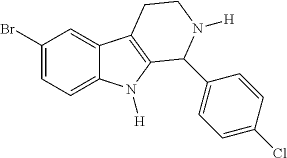 [SMILES: Clc1ccc(C2NCCc3c2[nH]c2ccc(Br)cc32)cc1] | 181 |
| 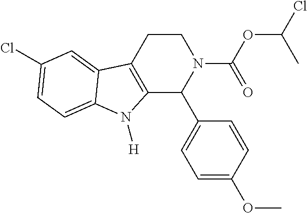 [SMILES: COc1ccc(C2c3[nH]c4ccc(Cl)cc4c3CCN2C(=O)OC(C)Cl)cc1] | 182 |
| 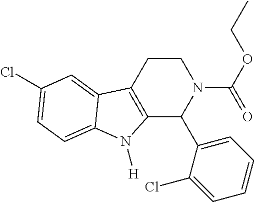 [SMILES: CCOC(=O)N1CCc2c([nH]c3ccc(Cl)cc23)C1c1ccccc1Cl] | 183 |
| 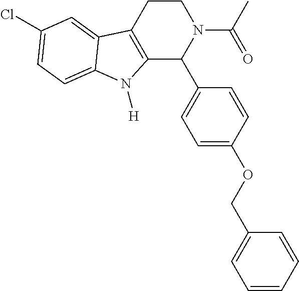 [SMILES: CC(=O)N1CCc2c([nH]c3ccc(Cl)cc23)C1c1ccc(OCc2ccccc2)cc1] | 184 |
| 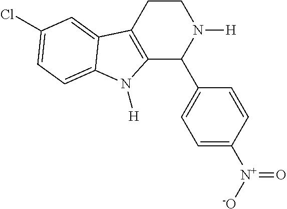 | 185 |
| 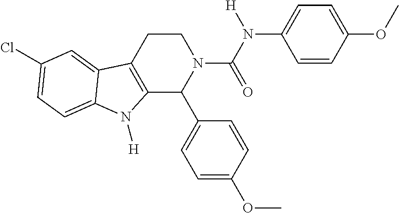 [SMILES: COc1ccc(NC(=O)N2CCc3c([nH]c4ccc(Cl)cc34)C2c2ccc(OC)cc2)cc1] | 186 |
| 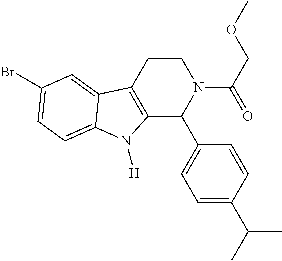 [SMILES: COCC(=O)N1CCc2c([nH]c3ccc(Br)cc23)C1c1ccc(C(C)C)cc1] | 187 |
| 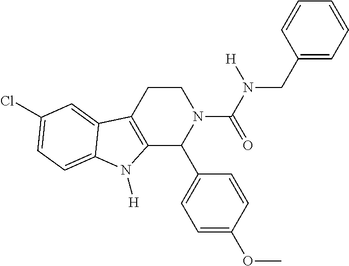 [SMILES: COc1ccc(C2c3[nH]c4ccc(Cl)cc4c3CCN2C(=O)NCc2ccccc2)cc1] | 188 |
| 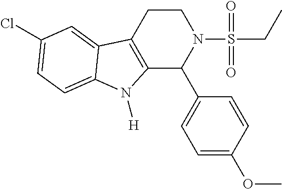 [SMILES: CCS(=O)(=O)N1CCc2c([nH]c3ccc(Cl)cc23)C1c1ccc(OC)cc1] | 189 |
| 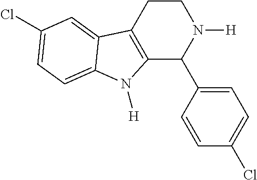 [SMILES: Clc1ccc(C2NCCc3c2[nH]c2ccc(Cl)cc32)cc1] | 190 |
| 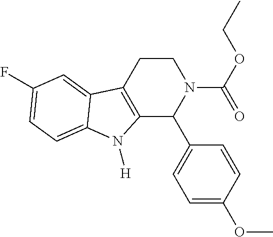 [SMILES: CCOC(=O)N1CCc2c([nH]c3ccc(F)cc23)C1c1ccc(OC)cc1] | 191 |
| 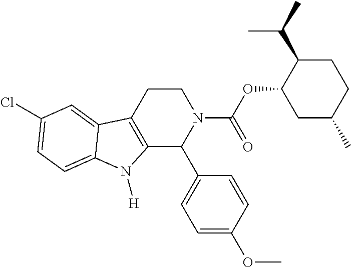 | 192 |
| 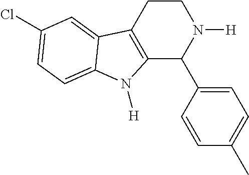 [SMILES: Cc1ccc(C2NCCc3c2[nH]c2ccc(Cl)cc32)cc1] | 193 |
| 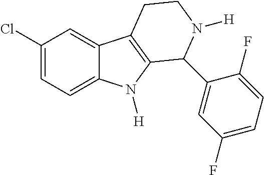 [SMILES: Fc1ccc(F)c(C2NCCc3c2[nH]c2ccc(Cl)cc32)c1] | 194 |
| 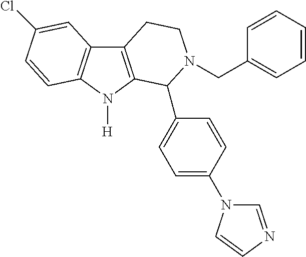 [SMILES: Clc1ccc2[nH]c3c(c2c1)CCN(Cc1ccccc1)C3c1ccc(-n2ccnc2)cc1] | 195 |
| 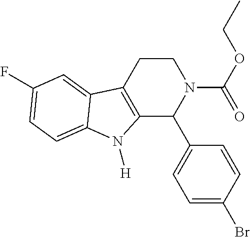 [SMILES: CCOC(=O)N1CCc2c([nH]c3ccc(F)cc23)C1c1ccc(Br)cc1] | 196 |
| 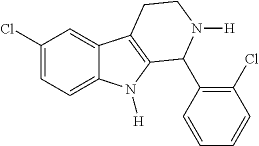 [SMILES: Clc1ccc2[nH]c3c(c2c1)CCNC3c1ccccc1Cl] | 197 |
| 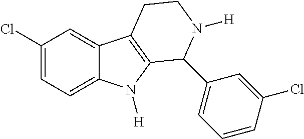 [SMILES: Clc1cccc(C2NCCc3c2[nH]c2ccc(Cl)cc32)c1] | 198 |
| 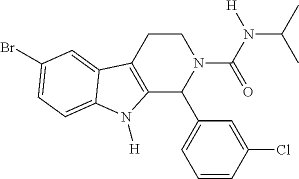 [SMILES: CC(C)NC(=O)N1CCc2c([nH]c3ccc(Br)cc23)C1c1cccc(Cl)c1] | 199 |
| 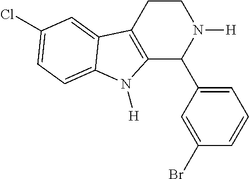 [SMILES: Clc1ccc2[nH]c3c(c2c1)CCNC3c1cccc(Br)c1] | 200 |
| 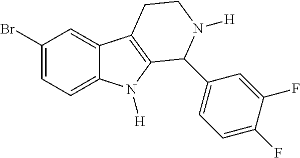 [SMILES: Fc1ccc(C2NCCc3c2[nH]c2ccc(Br)cc32)cc1F] | 201 |
| 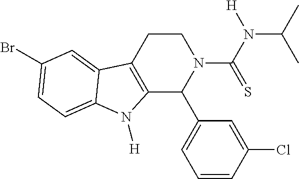 [SMILES: CC(C)NC(=S)N1CCc2c([nH]c3ccc(Br)cc23)C1c1cccc(Cl)c1] | 202 |
| 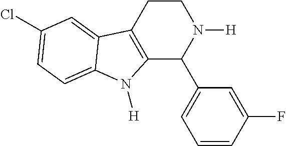 [SMILES: Fc1cccc(C2NCCc3c2[nH]c2ccc(Cl)cc32)c1] | 203 |
| 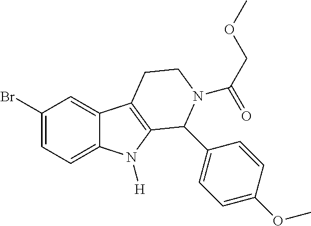 [SMILES: COCC(=O)N1CCc2c([nH]c3ccc(Br)cc23)C1c1ccc(OC)cc1] | 204 |
| 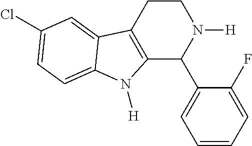 [SMILES: Fc1ccccc1C1NCCc2c1[nH]c1ccc(Cl)cc21] | 205 |
| 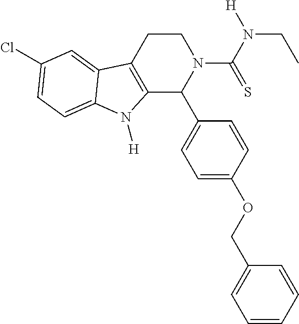 [SMILES: CCNC(=S)N1CCc2c([nH]c3ccc(Cl)cc23)C1c1ccc(OCc2ccccc2)cc1] | 206 |
| 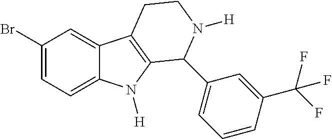 [SMILES: FC(F)(F)c1cccc(C2NCCc3c2[nH]c2ccc(Br)cc32)c1] | 207 |
| 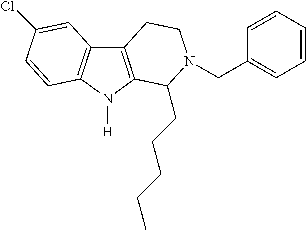 [SMILES: CCCCCC1c2[nH]c3ccc(Cl)cc3c2CCN1Cc1ccccc1] | 208 |
| 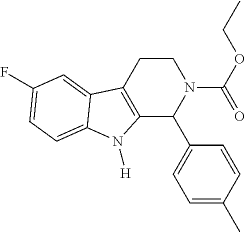 [SMILES: CCOC(=O)N1CCc2c([nH]c3ccc(F)cc23)C1c1ccc(C)cc1] | 209 |
| 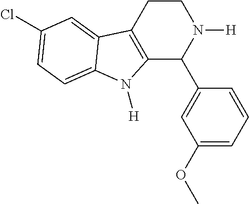 [SMILES: COc1cccc(C2NCCc3c2[nH]c2ccc(Cl)cc32)c1] | 210 |
| 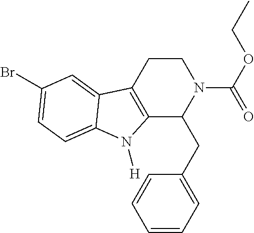 [SMILES: CCOC(=O)N1CCc2c([nH]c3ccc(Br)cc23)C1Cc1ccccc1] | 211 |
| 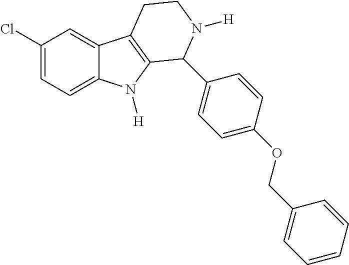 [SMILES: Clc1ccc2[nH]c3c(c2c1)CCNC3c1ccc(OCc2ccccc2)cc1] | 212 |
| 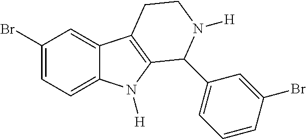 [SMILES: Brc1cccc(C2NCCc3c2[nH]c2ccc(Br)cc32)c1] | 213 |
| 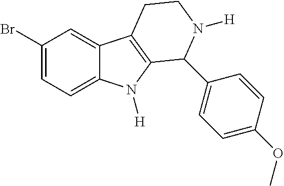 [SMILES: COc1ccc(C2NCCc3c2[nH]c2ccc(Br)cc32)cc1] | 214 |
| 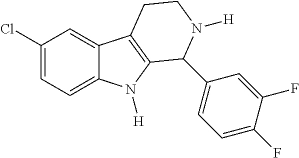 [SMILES: Fc1ccc(C2NCCc3c2[nH]c2ccc(Cl)cc32)cc1F] | 215 |
| 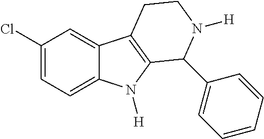 [SMILES: Clc1ccc2[nH]c3c(c2c1)CCNC3c1ccccc1] | 216 |
| 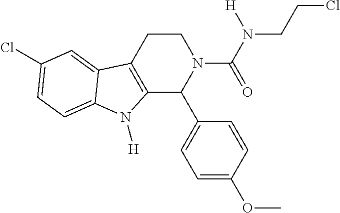 [SMILES: COc1ccc(C2c3[nH]c4ccc(Cl)cc4c3CCN2C(=O)NCCCl)cc1] | 217 |
| 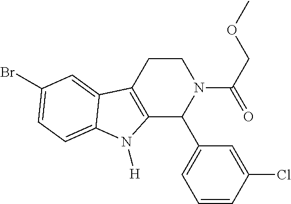 [SMILES: COCC(=O)N1CCc2c([nH]c3ccc(Br)cc23)C1c1cccc(Cl)c1] | 218 |
| 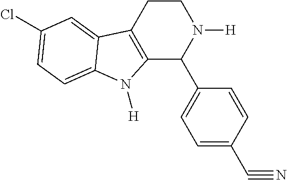 [SMILES: N#Cc1ccc(C2NCCc3c2[nH]c2ccc(Cl)cc32)cc1] | 220 |
| 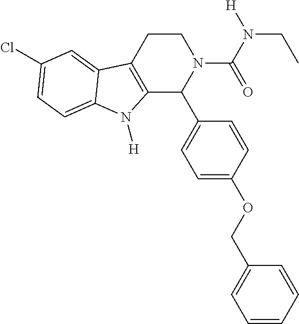 [SMILES: CCNC(=O)N1CCc2c([nH]c3ccc(Cl)cc23)C1c1ccc(OCc2ccccc2)cc1] | 221 |
| 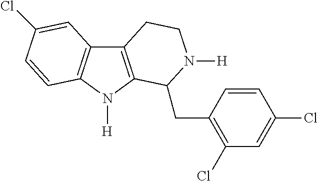 [SMILES: Clc1ccc(CC2NCCc3c2[nH]c2ccc(Cl)cc32)c(Cl)c1] | 222 |
| 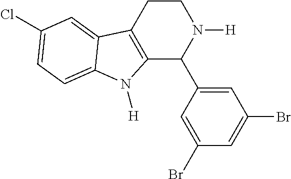 [SMILES: Clc1ccc2[nH]c3c(c2c1)CCNC3c1cc(Br)cc(Br)c1] | 223 |
| 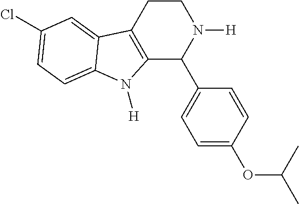 [SMILES: CC(C)Oc1ccc(C2NCCc3c2[nH]c2ccc(Cl)cc32)cc1] | 224 |
| 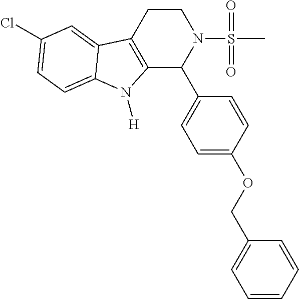 [SMILES: CS(=O)(=O)N1CCc2c([nH]c3ccc(Cl)cc23)C1c1ccc(OCc2ccccc2)cc1] | 225 |
| 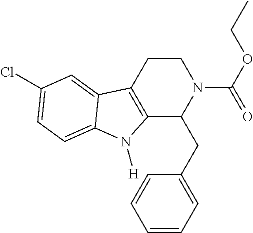 [SMILES: CCOC(=O)N1CCc2c([nH]c3ccc(Cl)cc23)C1Cc1ccccc1] | 226 |
| 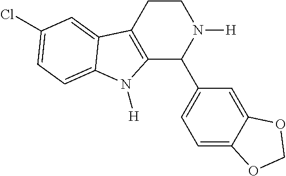 [SMILES: Clc1ccc2[nH]c3c(c2c1)CCNC3c1ccc2c(c1)OCO2] | 227 |
| 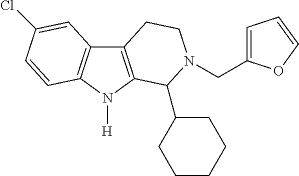 [SMILES: Clc1ccc2[nH]c3c(c2c1)CCN(Cc1ccco1)C3C1CCCCC1] | 228 |
| 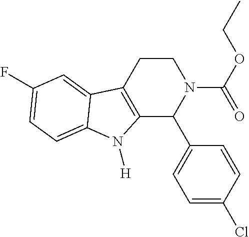 [SMILES: CCOC(=O)N1CCc2c([nH]c3ccc(F)cc23)C1c1ccc(Cl)cc1] | 229 |
| 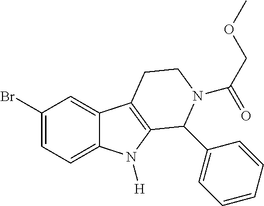 [SMILES: COCC(=O)N1CCc2c([nH]c3ccc(Br)cc23)C1c1ccccc1] | 230 |
| 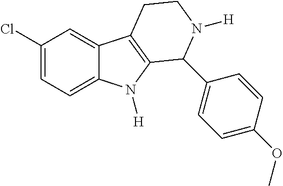 [SMILES: COc1ccc(C2NCCc3c2[nH]c2ccc(Cl)cc32)cc1] | 231 |
| 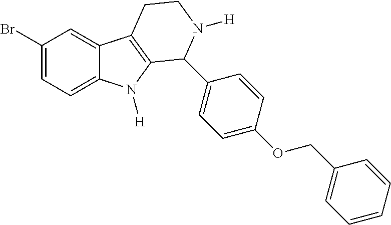 [SMILES: Brc1ccc2[nH]c3c(c2c1)CCNC3c1ccc(OCc2ccccc2)cc1] | 232 |
| 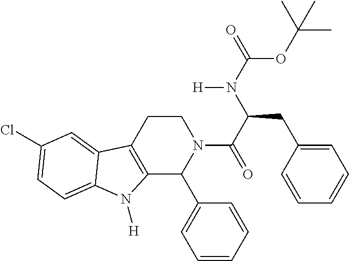 [SMILES: CC(C)(C)OC(=O)N[C@@H](Cc1ccccc1)C(=O)N1CCc2c([nH]c3ccc(Cl)cc23)C1c1ccccc1] | 233 |
| 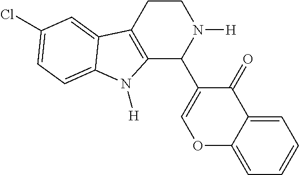 [SMILES: O=c1c(C2NCCc3c2[nH]c2ccc(Cl)cc32)coc2ccccc12] | 234 |
| 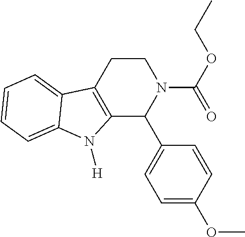 [SMILES: CCOC(=O)N1CCc2c([nH]c3ccccc23)C1c1ccc(OC)cc1] | 235 |
| 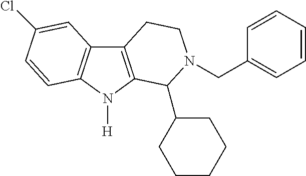 [SMILES: Clc1ccc2[nH]c3c(c2c1)CCN(Cc1ccccc1)C3C1CCCCC1] | 236 |
| 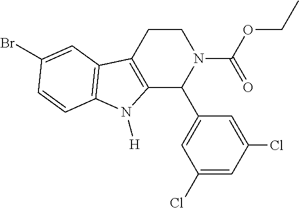 [SMILES: CCOC(=O)N1CCc2c([nH]c3ccc(Br)cc23)C1c1cc(Cl)cc(Cl)c1] | 237 |
| 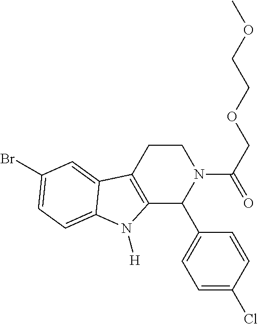 [SMILES: COCCOCC(=O)N1CCc2c([nH]c3ccc(Br)cc23)C1c1ccc(Cl)cc1] | 238 |
| 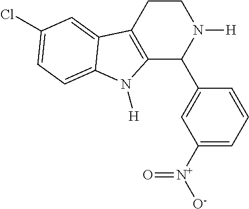 [SMILES: O=[N+]([O-])c1cccc(C2NCCc3c2[nH]c2ccc(Cl)cc32)c1] | 239 |
| 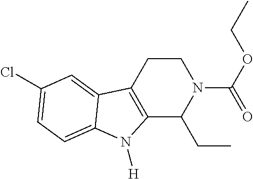 [SMILES: CCOC(=O)N1CCc2c([nH]c3ccc(Cl)cc23)C1CC] | 240 |
| 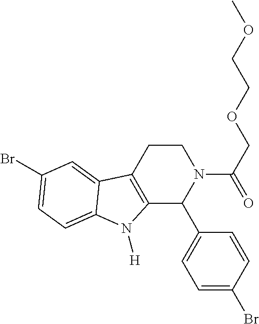 [SMILES: COCCOCC(=O)N1CCc2c([nH]c3ccc(Br)cc23)C1c1ccc(Br)cc1] | 241 |
| 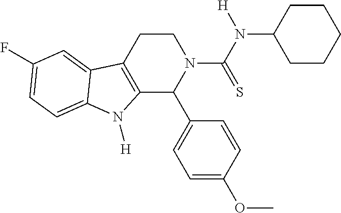 [SMILES: COc1ccc(C2c3[nH]c4ccc(F)cc4c3CCN2C(=S)NC2CCCCC2)cc1] | 242 |
| 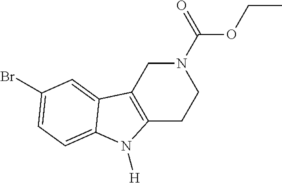 [SMILES: CCOC(=O)N1CCc2[nH]c3ccc(Br)cc3c2C1] | 243 |
| 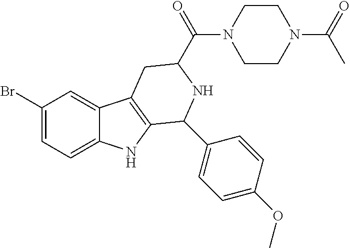 [SMILES: COc1ccc(C2NC(C(=O)N3CCN(C(C)=O)CC3)Cc3c2[nH]c2ccc(Br)cc32)cc1] | 244 |
| 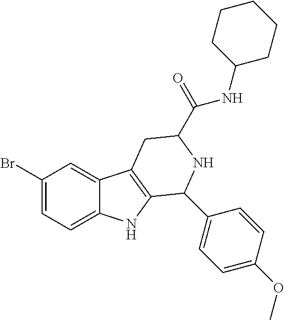 [SMILES: COc1ccc(C2NC(C(=O)NC3CCCCC3)Cc3c2[nH]c2ccc(Br)cc32)cc1] | 245 |
| 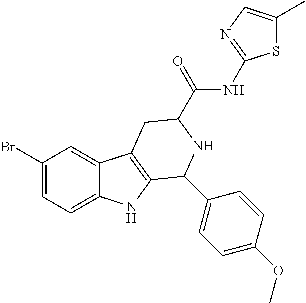 [SMILES: COc1ccc(C2NC(C(=O)Nc3ncc(C)s3)Cc3c2[nH]c2ccc(Br)cc32)cc1] | 246 |
| 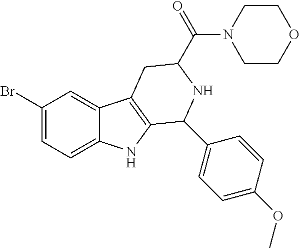 [SMILES: COc1ccc(C2NC(C(=O)N3CCOCC3)Cc3c2[nH]c2ccc(Br)cc32)cc1] | 247 |
| 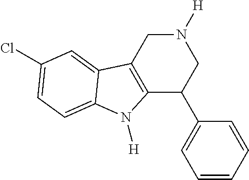 [SMILES: Clc1ccc2[nH]c3c(c2c1)CNCC3c1ccccc1] | 248 |
| 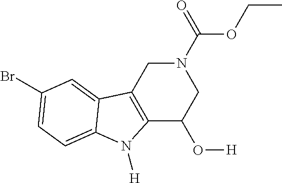 [SMILES: CCOC(=O)N1Cc2c([nH]c3ccc(Br)cc23)C(O)C1] | 249 |
| 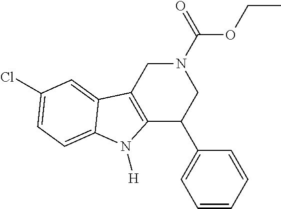 [SMILES: CCOC(=O)N1Cc2c([nH]c3ccc(Cl)cc23)C(c2ccccc2)C1] | 250 |
| 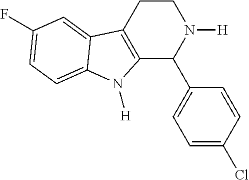 [SMILES: Fc1ccc2[nH]c3c(c2c1)CCNC3c1ccc(Cl)cc1] | 251 |
| 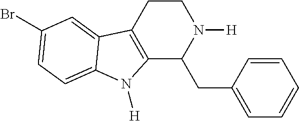 [SMILES: Brc1ccc2[nH]c3c(c2c1)CCNC3Cc1ccccc1] | 252 |
| 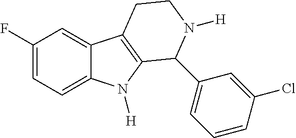 [SMILES: Fc1ccc2[nH]c3c(c2c1)CCNC3c1cccc(Cl)c1] | 253 |
| 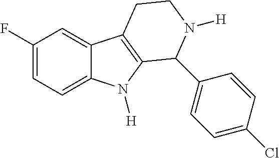 [SMILES: Fc1ccc2[nH]c3c(c2c1)CCNC3c1ccc(Cl)cc1] | 254 |
| 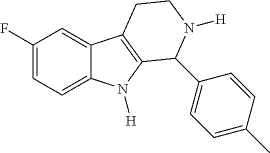 [SMILES: Cc1ccc(C2NCCc3c2[nH]c2ccc(F)cc32)cc1] | 255 |
| 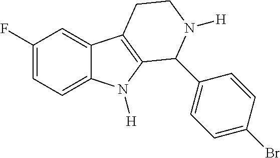 [SMILES: Fc1ccc2[nH]c3c(c2c1)CCNC3c1ccc(Br)cc1] | 256 |
| 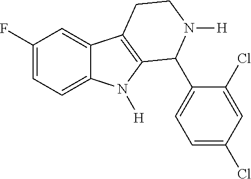 [SMILES: Fc1ccc2[nH]c3c(c2c1)CCNC3c1ccc(Cl)cc1Cl] | 257 |
| 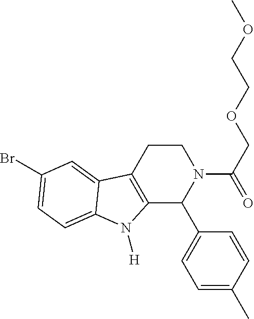 [SMILES: COCCOCC(=O)N1CCc2c([nH]c3ccc(Br)cc23)C1c1ccc(C)cc1] | 258 |
| 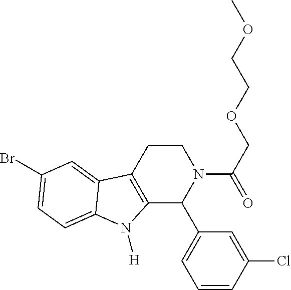 [SMILES: COCCOCC(=O)N1CCc2c([nH]c3ccc(Br)cc23)C1c1cccc(Cl)c1] | 259 |
| 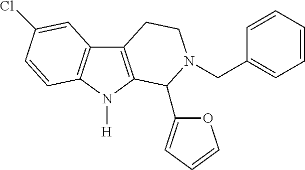 [SMILES: Clc1ccc2[nH]c3c(c2c1)CCN(Cc1ccccc1)C3c1ccco1] | 260 |
| 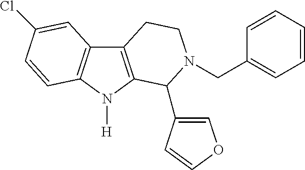 [SMILES: Clc1ccc2[nH]c3c(c2c1)CCN(Cc1ccccc1)C3c1ccoc1] | 261 |
| 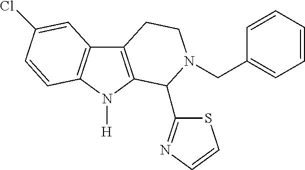 [SMILES: Clc1ccc2[nH]c3c(c2c1)CCN(Cc1ccccc1)C3c1nccs1] | 262 |
| 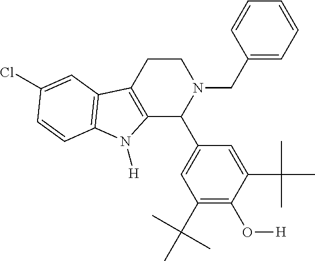 [SMILES: CC(C)(C)c1cc(C2c3[nH]c4ccc(Cl)cc4c3CCN2Cc2ccccc2)cc(C(C)(C)C)c1O] | 263 |
| 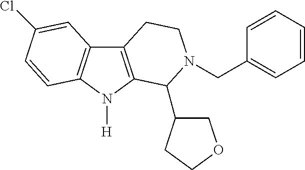 [SMILES: Clc1ccc2[nH]c3c(c2c1)CCN(Cc1ccccc1)C3C1CCOC1] | 264 |
| 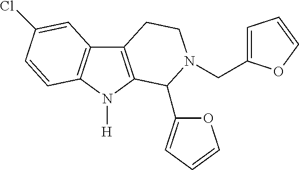 [SMILES: Clc1ccc2[nH]c3c(c2c1)CCN(Cc1ccco1)C3c1ccco1] | 265 |
| 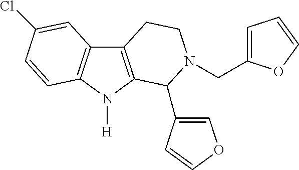 [SMILES: Clc1ccc2[nH]c3c(c2c1)CCN(Cc1ccco1)C3c1ccoc1] | 266 |
| 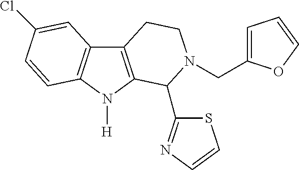 [SMILES: Clc1ccc2[nH]c3c(c2c1)CCN(Cc1ccco1)C3c1nccs1] | 267 |
| 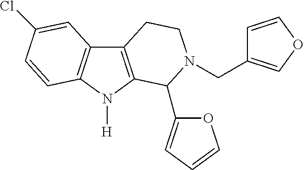 [SMILES: Clc1ccc2[nH]c3c(c2c1)CCN(Cc1ccoc1)C3c1ccco1] | 268 |
| 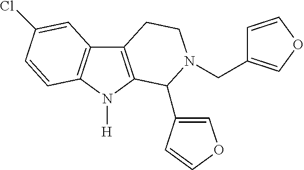 [SMILES: Clc1ccc2[nH]c3c(c2c1)CCN(Cc1ccoc1)C3c1ccoc1] | 269 |
| 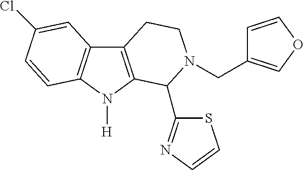 [SMILES: Clc1ccc2[nH]c3c(c2c1)CCN(Cc1ccoc1)C3c1nccs1] | 270 |
| 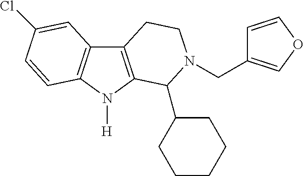 [SMILES: Clc1ccc2[nH]c3c(c2c1)CCN(Cc1ccoc1)C3C1CCCCC1] | 271 |
| 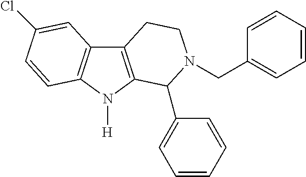 [SMILES: Clc1ccc2[nH]c3c(c2c1)CCN(Cc1ccccc1)C3c1ccccc1] | 272 |
| 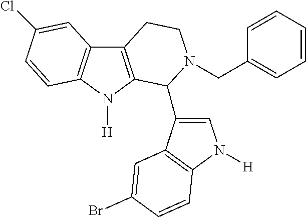 [SMILES: Clc1ccc2[nH]c3c(c2c1)CCN(Cc1ccccc1)C3c1c[nH]c2ccc(Br)cc12] | 273 |
| 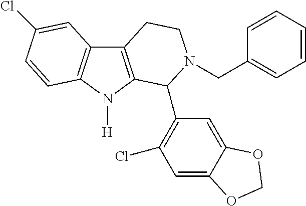 [SMILES: Clc1ccc2[nH]c3c(c2c1)CCN(Cc1ccccc1)C3c1cc2c(cc1Cl)OCO2] | 274 |
| 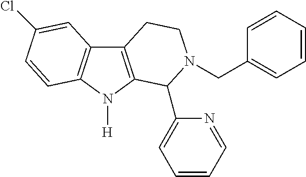 [SMILES: Clc1ccc2[nH]c3c(c2c1)CCN(Cc1ccccc1)C3c1ccccn1] | 275 |
| 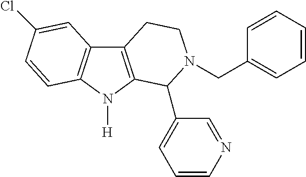 [SMILES: Clc1ccc2[nH]c3c(c2c1)CCN(Cc1ccccc1)C3c1cccnc1] | 276 |
| 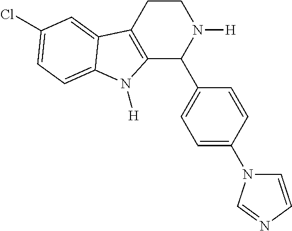 [SMILES: Clc1ccc2[nH]c3c(c2c1)CCNC3c1ccc(-n2ccnc2)cc1] | 277 |
| 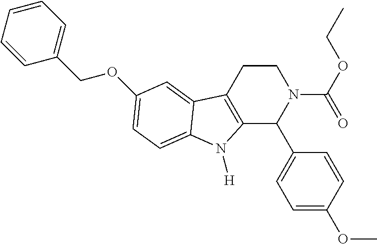 [SMILES: CCOC(=O)N1CCc2c([nH]c3ccc(OCc4ccccc4)cc23)C1c1ccc(OC)cc1] | 278 |
| 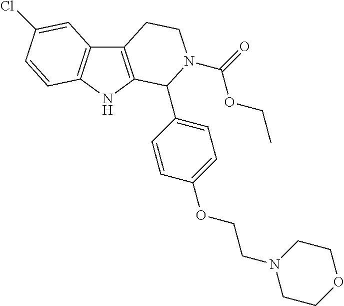 [SMILES: CCOC(=O)N1CCc2c([nH]c3ccc(Cl)cc23)C1c1ccc(OCCN2CCOCC2)cc1] | 279 |
| 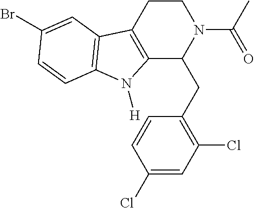 [SMILES: CC(=O)N1CCc2c([nH]c3ccc(Br)cc23)C1Cc1ccc(Cl)cc1Cl] | 280 |
| 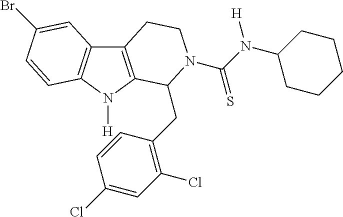 [SMILES: S=C(NC1CCCCC1)N1CCc2c([nH]c3ccc(Br)cc23)C1Cc1ccc(Cl)cc1Cl] | 281 |
| 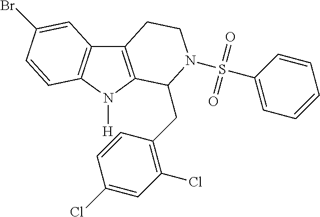 [SMILES: O=S(=O)(c1ccccc1)N1CCc2c([nH]c3ccc(Br)cc23)C1Cc1ccc(Cl)cc1Cl] | 282 |
| 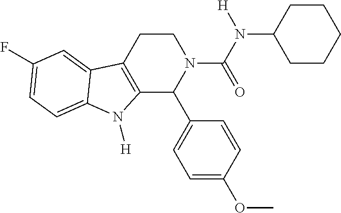 [SMILES: COc1ccc(C2c3[nH]c4ccc(F)cc4c3CCN2C(=O)NC2CCCCC2)cc1] | 283 |
| 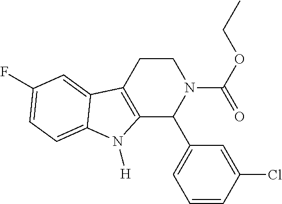 [SMILES: CCOC(=O)N1CCc2c([nH]c3ccc(F)cc23)C1c1cccc(Cl)c1] | 284 |
| 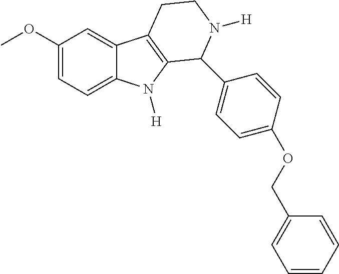 [SMILES: COc1ccc2[nH]c3c(c2c1)CCNC3c1ccc(OCc2ccccc2)cc1] | 285 |
| 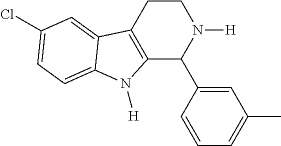 [SMILES: Cc1cccc(C2NCCc3c2[nH]c2ccc(Cl)cc32)c1] | 286 |
| 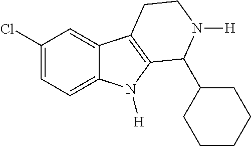 [SMILES: Clc1ccc2[nH]c3c(c2c1)CCNC3C1CCCCC1] | 287 |
| 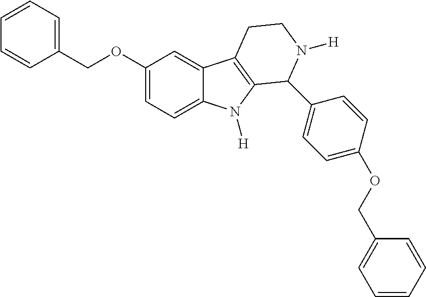 [SMILES: c1ccc(COc2ccc(C3NCCc4c3[nH]c3ccc(OCc5ccccc5)cc43)cc2)cc1] | 288 |
| 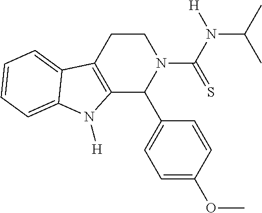 [SMILES: COc1ccc(C2c3[nH]c4ccccc4c3CCN2C(=S)NC(C)C)cc1] | 289 |
| 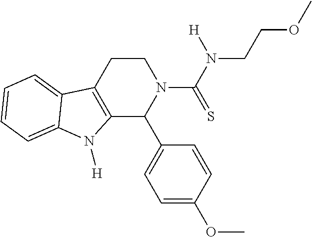 [SMILES: COCCNC(=S)N1CCc2c([nH]c3ccccc23)C1c1ccc(OC)cc1] | 290 |
| 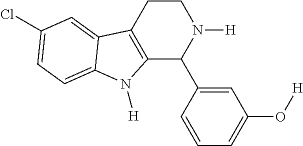 [SMILES: Oc1cccc(C2NCCc3c2[nH]c2ccc(Cl)cc32)c1] | 291 |
| 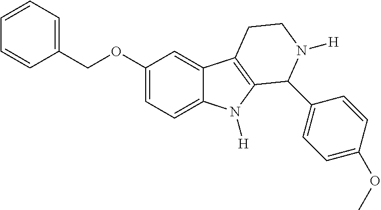 [SMILES: COc1ccc(C2NCCc3c2[nH]c2ccc(OCc4ccccc4)cc32)cc1] | 292 |
| 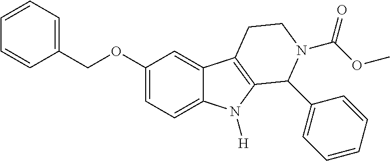 [SMILES: COC(=O)N1CCc2c([nH]c3ccc(OCc4ccccc4)cc23)C1c1ccccc1] | 293 |
| 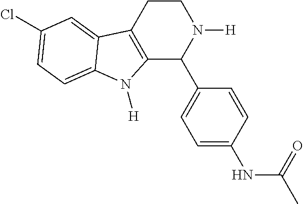 [SMILES: CC(=O)Nc1ccc(C2NCCc3c2[nH]c2ccc(Cl)cc32)cc1] | 294 |
| 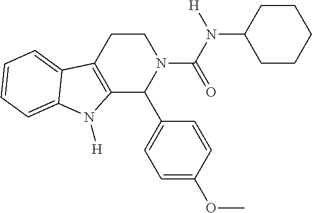 [SMILES: COc1ccc(C2c3[nH]c4ccccc4c3CCN2C(=O)NC2CCCCC2)cc1] | 295 |
| 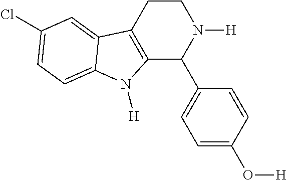 [SMILES: Oc1ccc(C2NCCc3c2[nH]c2ccc(Cl)cc32)cc1] | 296 |
| 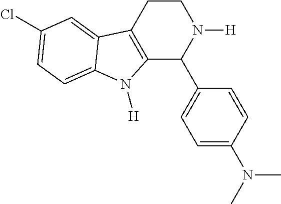 [SMILES: CN(C)c1ccc(C2NCCc3c2[nH]c2ccc(Cl)cc32)cc1] | 297 |
| 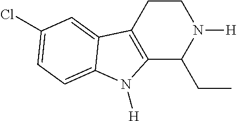 [SMILES: CCC1NCCc2c1[nH]c1ccc(Cl)cc21] | 298 |
| 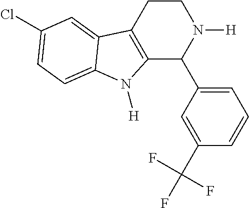 [SMILES: FC(F)(F)c1cccc(C2NCCc3c2[nH]c2ccc(Cl)cc32)c1] | 299 |
| 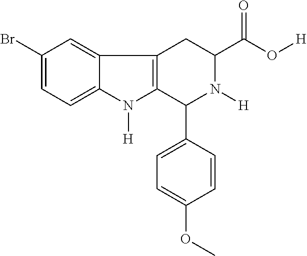 [SMILES: COc1ccc(C2NC(C(=O)O)Cc3c2[nH]c2ccc(Br)cc32)cc1] | 300 |
| 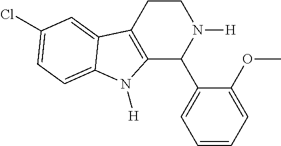 [SMILES: COc1ccccc1C1NCCc2c1[nH]c1ccc(Cl)cc21] | 301 |
| 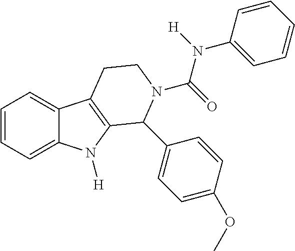 [SMILES: COc1ccc(C2c3[nH]c4ccccc4c3CCN2C(=O)Nc2ccccc2)cc1] | 302 |
| 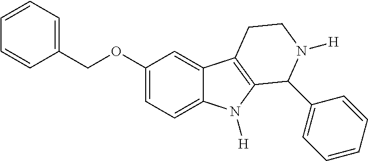 [SMILES: c1ccc(COc2ccc3[nH]c4c(c3c2)CCNC4c2ccccc2)cc1] | 303 |
| 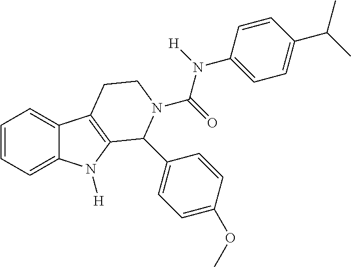 [SMILES: COc1ccc(C2c3[nH]c4ccccc4c3CCN2C(=O)Nc2ccc(C(C)C)cc2)cc1] | 304 |
| 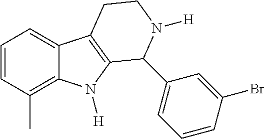 [SMILES: Cc1cccc2c3c([nH]c12)C(c1cccc(Br)c1)NCC3] | 305 |
| 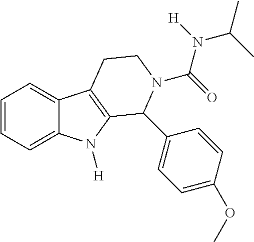 [SMILES: COc1ccc(C2c3[nH]c4ccccc4c3CCN2C(=O)NC(C)C)cc1] | 306 |
| 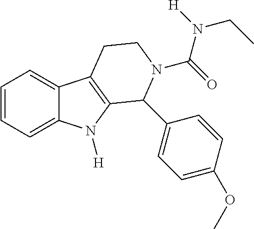 [SMILES: CCNC(=O)N1CCc2c([nH]c3ccccc23)C1c1ccc(OC)cc1] | 307 |
| 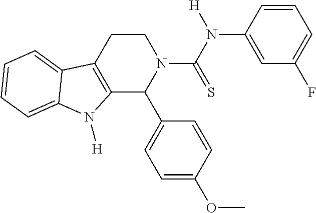 [SMILES: COc1ccc(C2c3[nH]c4ccccc4c3CCN2C(=S)Nc2cccc(F)c2)cc1] | 308 |
| 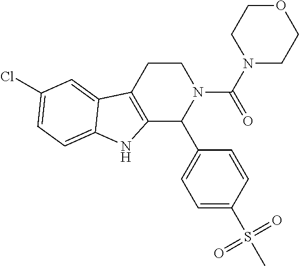 [SMILES: CS(=O)(=O)c1ccc(C2c3[nH]c4ccc(Cl)cc4c3CCN2C(=O)N2CCOCC2)cc1] | 309 |
| 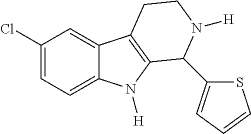 [SMILES: Clc1ccc2[nH]c3c(c2c1)CCNC3c1cccs1] | 310 |
| 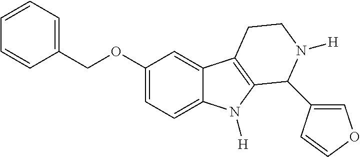 [SMILES: c1ccc(COc2ccc3[nH]c4c(c3c2)CCNC4c2ccoc2)cc1] | 311 |
| 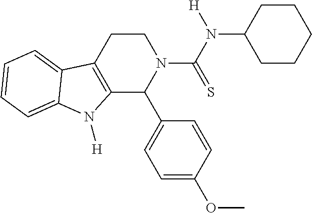 [SMILES: COc1ccc(C2c3[nH]c4ccccc4c3CCN2C(=S)NC2CCCCC2)cc1] | 312 |
| 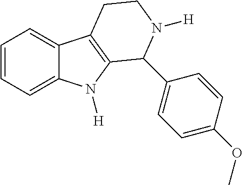 [SMILES: COc1ccc(C2NCCc3c2[nH]c2ccccc32)cc1] | 313 |
| 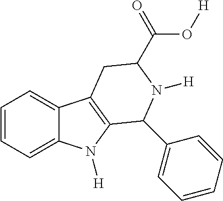 [SMILES: O=C(O)C1Cc2c([nH]c3ccccc23)C(c2ccccc2)N1] | 314 |
| 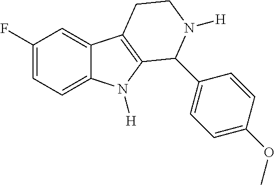 [SMILES: COc1ccc(C2NCCc3c2[nH]c2ccc(F)cc32)cc1] | 315 |
| 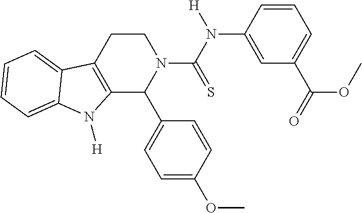 [SMILES: COC(=O)c1cccc(NC(=S)N2CCc3c([nH]c4ccccc34)C2c2ccc(OC)cc2)c1] | 316 |
| 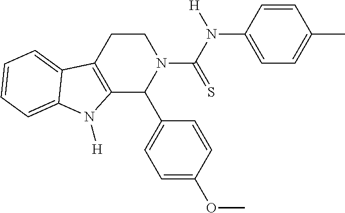 [SMILES: COc1ccc(C2c3[nH]c4ccccc4c3CCN2C(=S)Nc2ccc(C)cc2)cc1] | 317 |
| 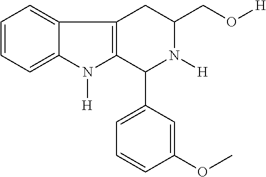 [SMILES: COc1cccc(C2NC(CO)Cc3c2[nH]c2ccccc32)c1] | 318 |
| 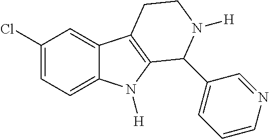 [SMILES: Clc1ccc2[nH]c3c(c2c1)CCNC3c1cccnc1] | 319 |
| 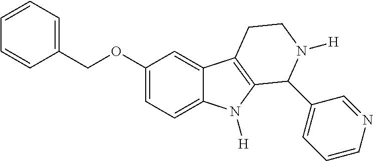 [SMILES: c1ccc(COc2ccc3[nH]c4c(c3c2)CCNC4c2cccnc2)cc1] | 320 |
| 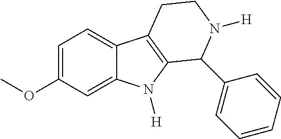 [SMILES: COc1ccc2c3c([nH]c2c1)C(c1ccccc1)NCC3] | 321 |
| 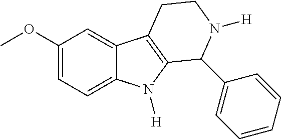 [SMILES: COc1ccc2[nH]c3c(c2c1)CCNC3c1ccccc1] | 322 |
| 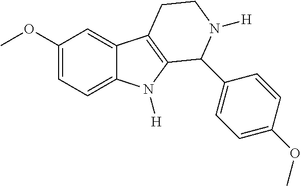 [SMILES: COc1ccc(C2NCCc3c2[nH]c2ccc(OC)cc32)cc1] | 323 |
| 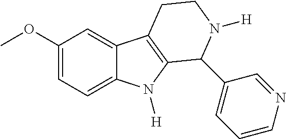 [SMILES: COc1ccc2[nH]c3c(c2c1)CCNC3c1cccnc1] | 324 |
| 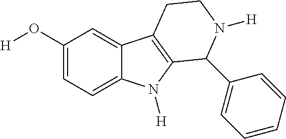 [SMILES: Oc1ccc2[nH]c3c(c2c1)CCNC3c1ccccc1] | 325 |
| 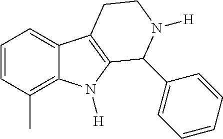 [SMILES: Cc1cccc2c3c([nH]c12)C(c1ccccc1)NCC3] | 326 |
| 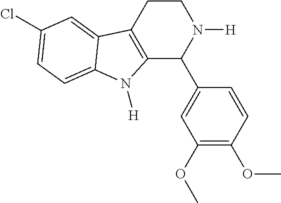 [SMILES: COc1ccc(C2NCCc3c2[nH]c2ccc(Cl)cc32)cc1OC] | 327 |
| 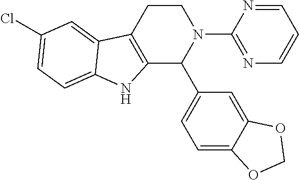 [SMILES: Clc1ccc2[nH]c3c(c2c1)CCN(c1ncccn1)C3c1ccc2c(c1)OCO2] | 328 |
| 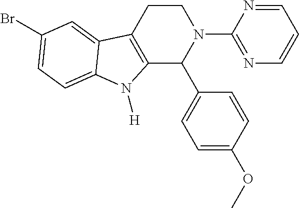 [SMILES: COc1ccc(C2c3[nH]c4ccc(Br)cc4c3CCN2c2ncccn2)cc1] | 329 |
| 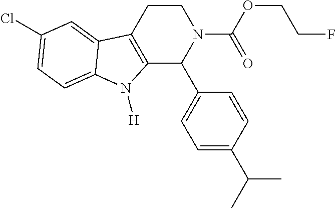 [SMILES: CC(C)c1ccc(C2c3[nH]c4ccc(Cl)cc4c3CCN2C(=O)OCCF)cc1] | 330 |
| 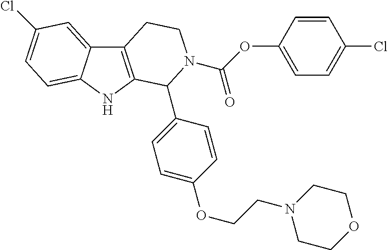 [SMILES: O=C(Oc1ccc(Cl)cc1)N1CCc2c([nH]c3ccc(Cl)cc23)C1c1ccc(OCCN2CCOCC2)cc1] | 331 |
| 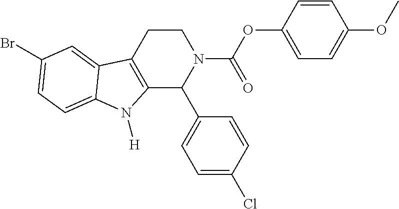 [SMILES: COc1ccc(OC(=O)N2CCc3c([nH]c4ccc(Br)cc34)C2c2ccc(Cl)cc2)cc1] | 332 |
| 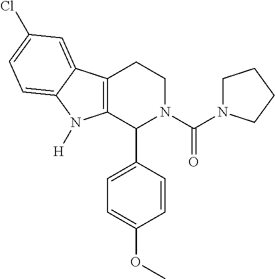 [SMILES: COc1ccc(C2c3[nH]c4ccc(Cl)cc4c3CCN2C(=O)N2CCCC2)cc1] | 333 |
| 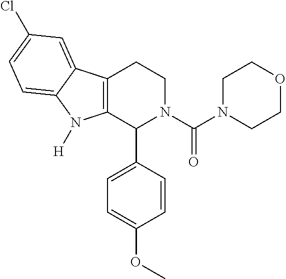 [SMILES: COc1ccc(C2c3[nH]c4ccc(Cl)cc4c3CCN2C(=O)N2CCOCC2)cc1] | 334 |
| 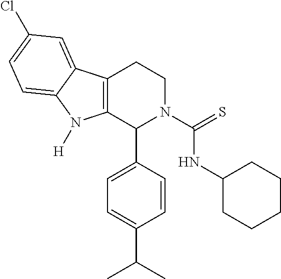 [SMILES: CC(C)c1ccc(C2c3[nH]c4ccc(Cl)cc4c3CCN2C(=S)NC2CCCCC2)cc1] | 335 |
| 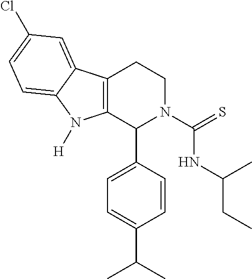 [SMILES: CCC(C)NC(=S)N1CCc2c([nH]c3ccc(Cl)cc23)C1c1ccc(C(C)C)cc1] | 336 |
| 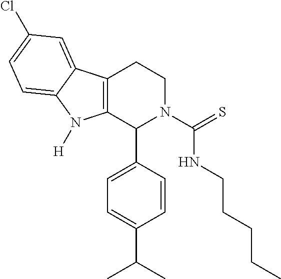 [SMILES: CCCCCNC(=S)N1CCc2c([nH]c3ccc(Cl)cc23)C1c1ccc(C(C)C)cc1] | 337 |
| 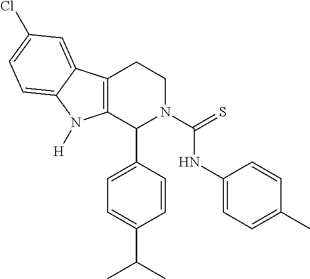 [SMILES: Cc1ccc(NC(=S)N2CCc3c([nH]c4ccc(Cl)cc34)C2c2ccc(C(C)C)cc2)cc1] | 338 |
| 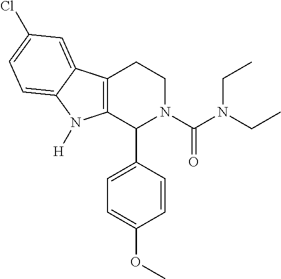 [SMILES: CCN(CC)C(=O)N1CCc2c([nH]c3ccc(Cl)cc23)C1c1ccc(OC)cc1] | 339 |
| 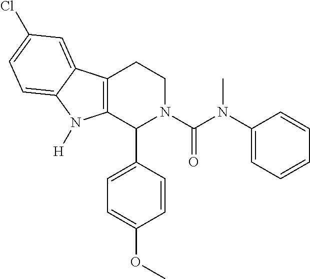 [SMILES: COc1ccc(C2c3[nH]c4ccc(Cl)cc4c3CCN2C(=O)N(C)c2ccccc2)cc1] | 340 |
| 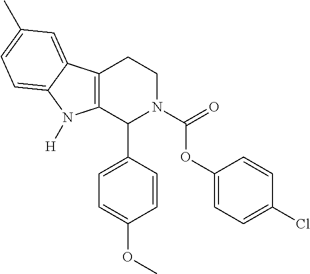 [SMILES: COc1ccc(C2c3[nH]c4ccc(C)cc4c3CCN2C(=O)Oc2ccc(Cl)cc2)cc1] | 341 |
| 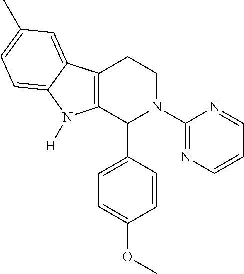 [SMILES: COc1ccc(C2c3[nH]c4ccc(C)cc4c3CCN2c2ncccn2)cc1] | 342 |
| 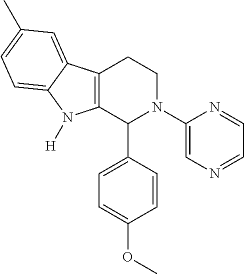 [SMILES: COc1ccc(C2c3[nH]c4ccc(C)cc4c3CCN2c2cnccn2)cc1] | 343 |
| 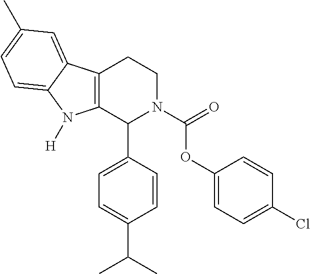 [SMILES: Cc1ccc2[nH]c3c(c2c1)CCN(C(=O)Oc1ccc(Cl)cc1)C3c1ccc(C(C)C)cc1] | 344 |
| 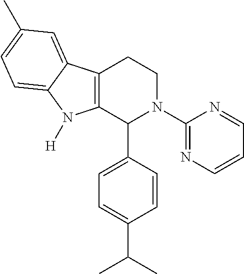 [SMILES: Cc1ccc2[nH]c3c(c2c1)CCN(c1ncccn1)C3c1ccc(C(C)C)cc1] | 345 |
| 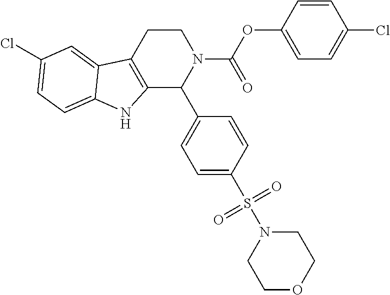 [SMILES: O=C(Oc1ccc(Cl)cc1)N1CCc2c([nH]c3ccc(Cl)cc23)C1c1ccc(S(=O)(=O)N2CCOCC2)cc1] | 346 |
| 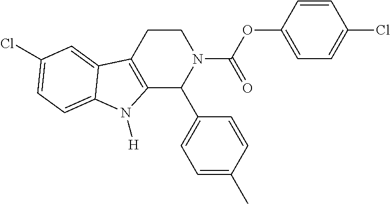 [SMILES: Cc1ccc(C2c3[nH]c4ccc(Cl)cc4c3CCN2C(=O)Oc2ccc(Cl)cc2)cc1] | 347 |
| 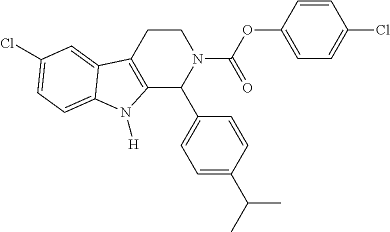 [SMILES: CC(C)c1ccc(C2c3[nH]c4ccc(Cl)cc4c3CCN2C(=O)Oc2ccc(Cl)cc2)cc1] | 348 |
| 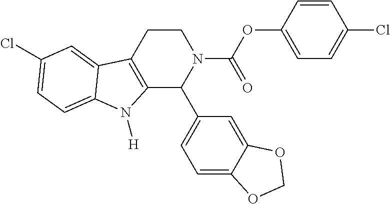 [SMILES: O=C(Oc1ccc(Cl)cc1)N1CCc2c([nH]c3ccc(Cl)cc23)C1c1ccc2c(c1)OCO2] | 349 |
| 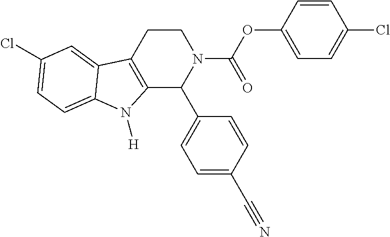 [SMILES: N#Cc1ccc(C2c3[nH]c4ccc(Cl)cc4c3CCN2C(=O)Oc2ccc(Cl)cc2)cc1] | 350 |
| 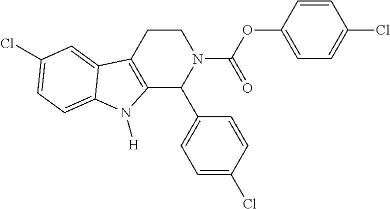 [SMILES: O=C(Oc1ccc(Cl)cc1)N1CCc2c([nH]c3ccc(Cl)cc23)C1c1ccc(Cl)cc1] | 351 |
| 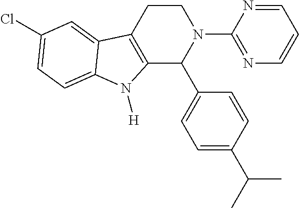 [SMILES: CC(C)c1ccc(C2c3[nH]c4ccc(Cl)cc4c3CCN2c2ncccn2)cc1] | 352 |
| 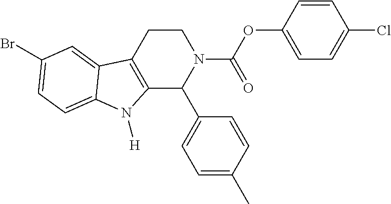 [SMILES: Cc1ccc(C2c3[nH]c4ccc(Br)cc4c3CCN2C(=O)Oc2ccc(Cl)cc2)cc1] | 353 |
| 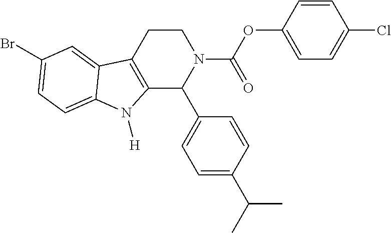 [SMILES: CC(C)c1ccc(C2c3[nH]c4ccc(Br)cc4c3CCN2C(=O)Oc2ccc(Cl)cc2)cc1] | 354 |
| 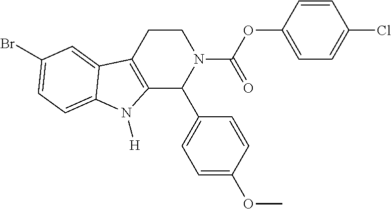 [SMILES: COc1ccc(C2c3[nH]c4ccc(Br)cc4c3CCN2C(=O)Oc2ccc(Cl)cc2)cc1] | 355 |
| 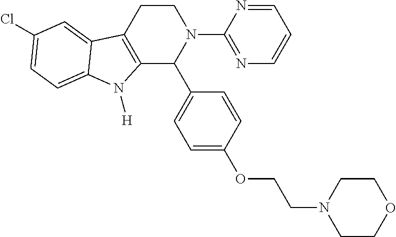 [SMILES: Clc1ccc2[nH]c3c(c2c1)CCN(c1ncccn1)C3c1ccc(OCCN2CCOCC2)cc1] | 356 |
| 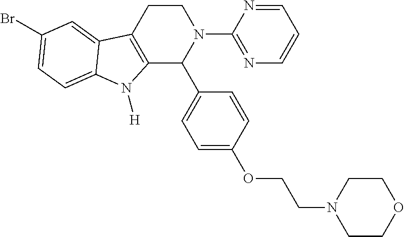 [SMILES: Brc1ccc2[nH]c3c(c2c1)CCN(c1ncccn1)C3c1ccc(OCCN2CCOCC2)cc1] | 357 |
| 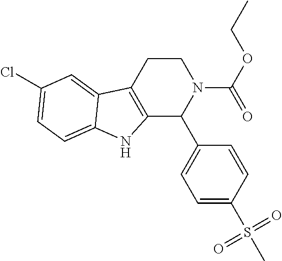 [SMILES: CCOC(=O)N1CCc2c([nH]c3ccc(Cl)cc23)C1c1ccc(S(C)(=O)=O)cc1] | 358 |
| 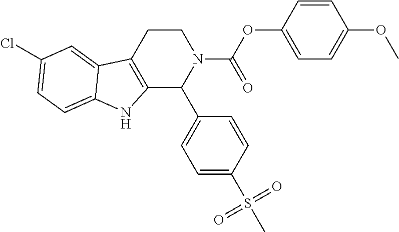 [SMILES: COc1ccc(OC(=O)N2CCc3c([nH]c4ccc(Cl)cc34)C2c2ccc(S(C)(=O)=O)cc2)cc1] | 359 |
| 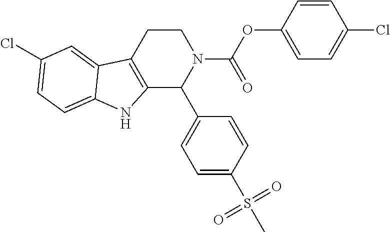 [SMILES: CS(=O)(=O)c1ccc(C2c3[nH]c4ccc(Cl)cc4c3CCN2C(=O)Oc2ccc(Cl)cc2)cc1] | 360 |
| 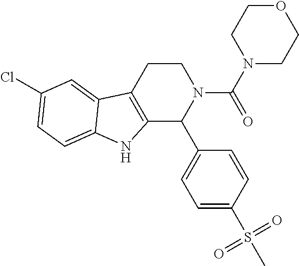 [SMILES: CS(=O)(=O)c1ccc(C2c3[nH]c4ccc(Cl)cc4c3CCN2C(=O)N2CCOCC2)cc1] | 361 |
| 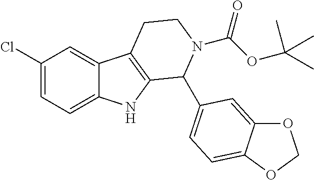 [SMILES: CC(C)(C)OC(=O)N1CCc2c([nH]c3ccc(Cl)cc23)C1c1ccc2c(c1)OCO2] | 362 |
| 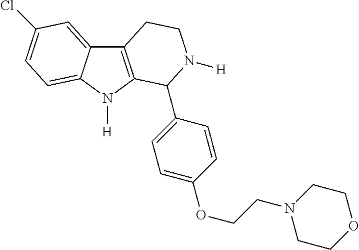 [SMILES: Clc1ccc2[nH]c3c(c2c1)CCNC3c1ccc(OCCN2CCOCC2)cc1] | 363 |
| 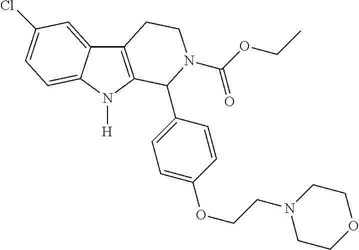 [SMILES: CCOC(=O)N1CCc2c([nH]c3ccc(Cl)cc23)C1c1ccc(OCCN2CCOCC2)cc1] | 364 |
| 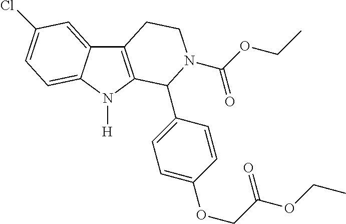 [SMILES: CCOC(=O)COc1ccc(C2c3[nH]c4ccc(Cl)cc4c3CCN2C(=O)OCC)cc1] | 365 |
| 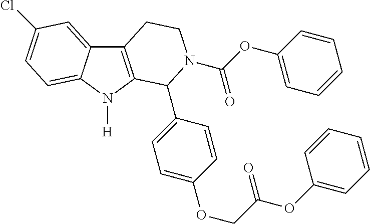 [SMILES: O=C(COc1ccc(C2c3[nH]c4ccc(Cl)cc4c3CCN2C(=O)Oc2ccccc2)cc1)Oc1ccccc1] | 366 |
| 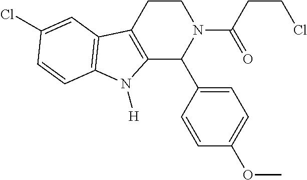 [SMILES: COc1ccc(C2c3[nH]c4ccc(Cl)cc4c3CCN2C(=O)CCCl)cc1] | 367 |
| 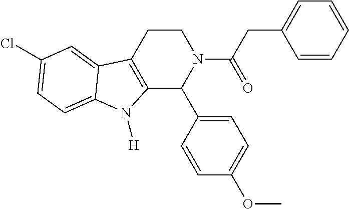 [SMILES: COc1ccc(C2c3[nH]c4ccc(Cl)cc4c3CCN2C(=O)Cc2ccccc2)cc1] | 368 |
| 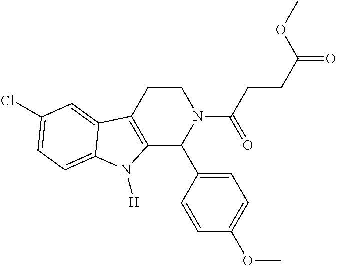 [SMILES: COC(=O)CCC(=O)N1CCc2c([nH]c3ccc(Cl)cc23)C1c1ccc(OC)cc1] | 369 |
| 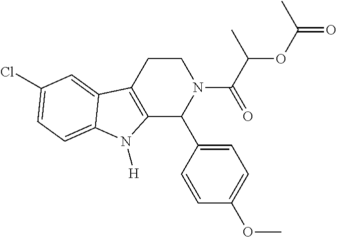 [SMILES: COc1ccc(C2c3[nH]c4ccc(Cl)cc4c3CCN2C(=O)C(C)OC(C)=O)cc1] | 370 |
| 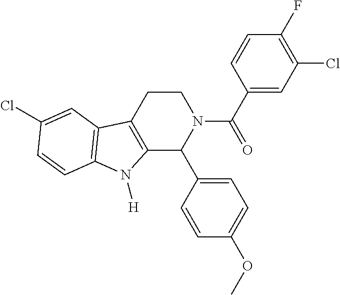 [SMILES: COc1ccc(C2c3[nH]c4ccc(Cl)cc4c3CCN2C(=O)c2ccc(F)c(Cl)c2)cc1] | 371 |
| 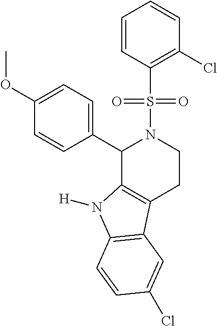 [SMILES: COc1ccc(C2c3[nH]c4ccc(Cl)cc4c3CCN2S(=O)(=O)c2ccccc2Cl)cc1] | 372 |
| 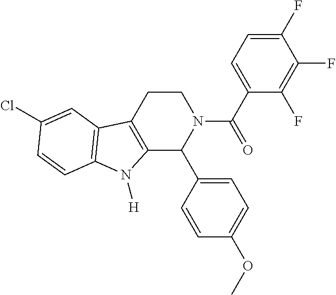 [SMILES: COc1ccc(C2c3[nH]c4ccc(Cl)cc4c3CCN2C(=O)c2ccc(F)c(F)c2F)cc1] | 373 |
| 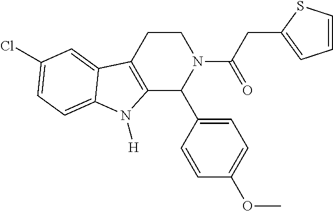 [SMILES: COc1ccc(C2c3[nH]c4ccc(Cl)cc4c3CCN2C(=O)Cc2cccs2)cc1] | 374 |
| 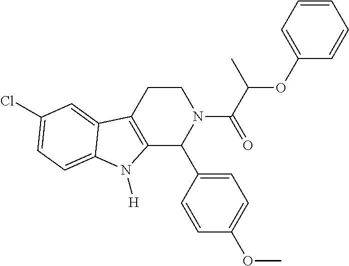 [SMILES: COc1ccc(C2c3[nH]c4ccc(Cl)cc4c3CCN2C(=O)C(C)Oc2ccccc2)cc1] | 375 |
| 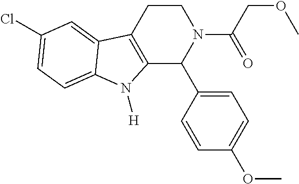 [SMILES: COCC(=O)N1CCc2c([nH]c3ccc(Cl)cc23)C1c1ccc(OC)cc1] | 376 |
| 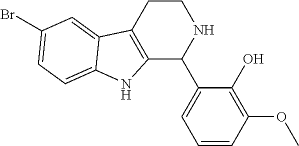 [SMILES: COc1cccc(C2NCCc3c2[nH]c2ccc(Br)cc32)c1O] | 377 |
| 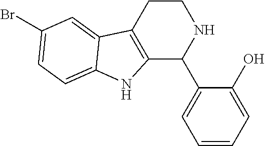 [SMILES: Oc1ccccc1C1NCCc2c1[nH]c1ccc(Br)cc21] | 378 |
| 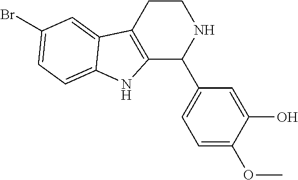 [SMILES: COc1ccc(C2NCCc3c2[nH]c2ccc(Br)cc32)cc1O] | 379 |
| 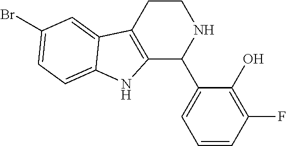 [SMILES: Oc1c(F)cccc1C1NCCc2c1[nH]c1ccc(Br)cc21] | 380 |
| 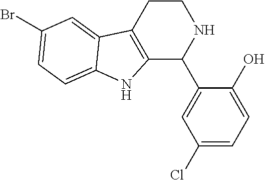 [SMILES: Oc1ccc(Cl)cc1C1NCCc2c1[nH]c1ccc(Br)cc21] | 381 |
| 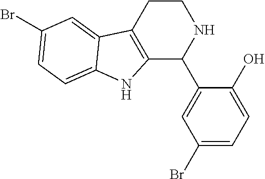 [SMILES: Oc1ccc(Br)cc1C1NCCc2c1[nH]c1ccc(Br)cc21] | 382 |
| 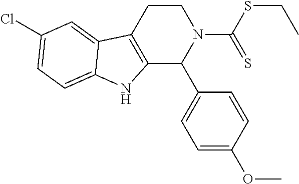 [SMILES: CCSC(=S)N1CCc2c([nH]c3ccc(Cl)cc23)C1c1ccc(OC)cc1] | 383 |
| 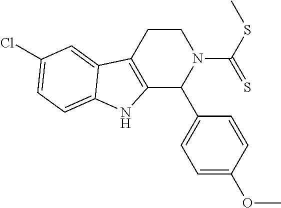 [SMILES: COc1ccc(C2c3[nH]c4ccc(Cl)cc4c3CCN2C(=S)SC)cc1] | 384 |
| 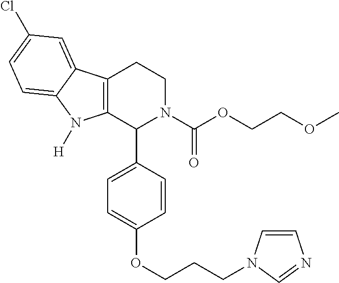 [SMILES: COCCOC(=O)N1CCc2c([nH]c3ccc(Cl)cc23)C1c1ccc(OCCCn2ccnc2)cc1] | 385 |
| 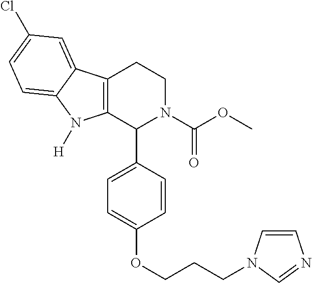 [SMILES: COC(=O)N1CCc2c([nH]c3ccc(Cl)cc23)C1c1ccc(OCCCn2ccnc2)cc1] | 386 |
| 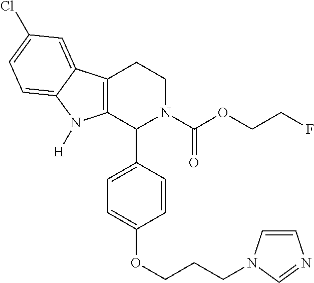 [SMILES: O=C(OCCF)N1CCc2c([nH]c3ccc(Cl)cc23)C1c1ccc(OCCCn2ccnc2)cc1] | 387 |
| 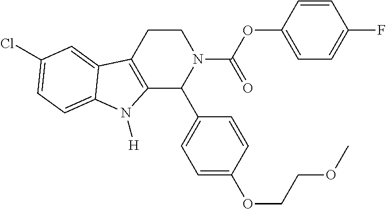 [SMILES: COCCOc1ccc(C2c3[nH]c4ccc(Cl)cc4c3CCN2C(=O)Oc2ccc(F)cc2)cc1] | 388 |
| 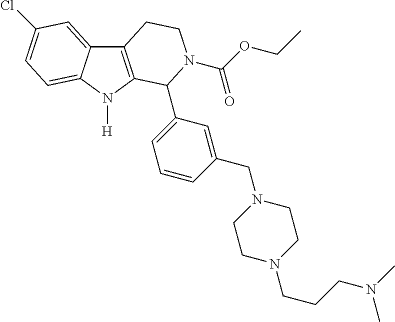 [SMILES: CCOC(=O)N1CCc2c([nH]c3ccc(Cl)cc23)C1c1cccc(CN2CCN(CCCN(C)C)CC2)c1] | 389 |
| 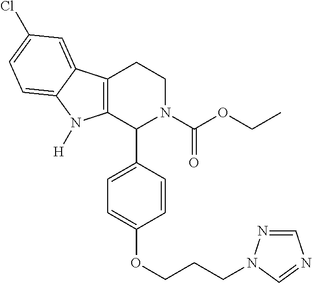 [SMILES: CCOC(=O)N1CCc2c([nH]c3ccc(Cl)cc23)C1c1ccc(OCCCn2cncn2)cc1] | 390 |
| 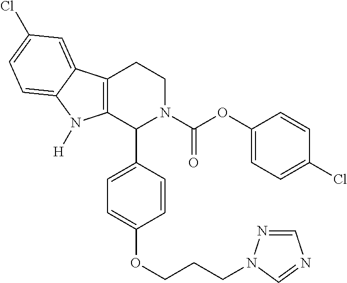 [SMILES: O=C(Oc1ccc(Cl)cc1)N1CCc2c([nH]c3ccc(Cl)cc23)C1c1ccc(OCCCn2cncn2)cc1] | 391 |
| 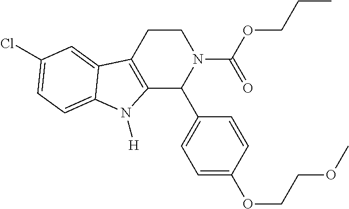 [SMILES: CCCOC(=O)N1CCc2c([nH]c3ccc(Cl)cc23)C1c1ccc(OCCOC)cc1] | 392 |
| 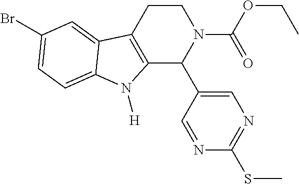 [SMILES: CCOC(=O)N1CCc2c([nH]c3ccc(Br)cc23)C1c1cnc(SC)nc1] | 393 |
| 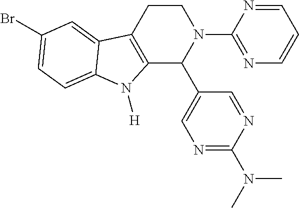 [SMILES: CN(C)c1ncc(C2c3[nH]c4ccc(Br)cc4c3CCN2c2ncccn2)cn1] | 394 |
| 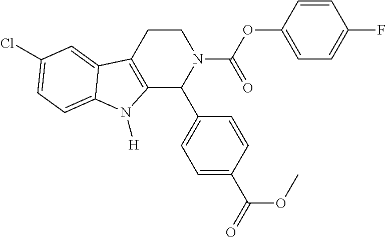 [SMILES: COC(=O)c1ccc(C2c3[nH]c4ccc(Cl)cc4c3CCN2C(=O)Oc2ccc(F)cc2)cc1] | 395 |
| 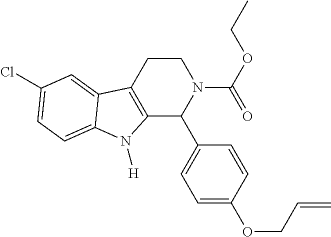 [SMILES: C=CCOc1ccc(C2c3[nH]c4ccc(Cl)cc4c3CCN2C(=O)OCC)cc1] | 396 |
| 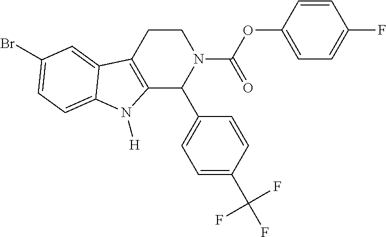 [SMILES: O=C(Oc1ccc(F)cc1)N1CCc2c([nH]c3ccc(Br)cc23)C1c1ccc(C(F)(F)F)cc1] | 397 |
| 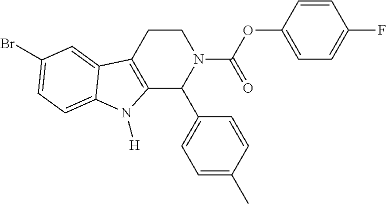 [SMILES: Cc1ccc(C2c3[nH]c4ccc(Br)cc4c3CCN2C(=O)Oc2ccc(F)cc2)cc1] | 398 |
| 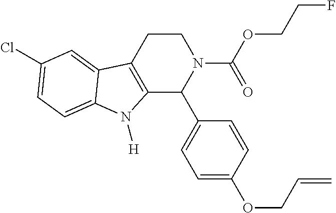 [SMILES: C=CCOc1ccc(C2c3[nH]c4ccc(Cl)cc4c3CCN2C(=O)OCCF)cc1] | 399 |
| 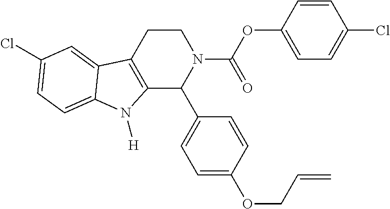 [SMILES: C=CCOc1ccc(C2c3[nH]c4ccc(Cl)cc4c3CCN2C(=O)Oc2ccc(Cl)cc2)cc1] | 400 |
| 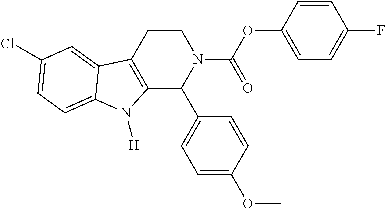 [SMILES: COc1ccc(C2c3[nH]c4ccc(Cl)cc4c3CCN2C(=O)Oc2ccc(F)cc2)cc1] | 401 |
| 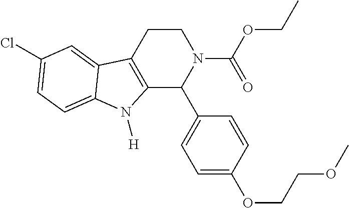 [SMILES: CCOC(=O)N1CCc2c([nH]c3ccc(Cl)cc23)C1c1ccc(OCCOC)cc1] | 402 |
| 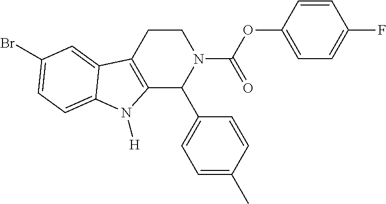 [SMILES: Cc1ccc(C2c3[nH]c4ccc(Br)cc4c3CCN2C(=O)Oc2ccc(F)cc2)cc1] | 403 |
| 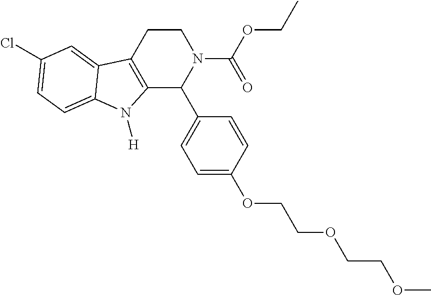 [SMILES: CCOC(=O)N1CCc2c([nH]c3ccc(Cl)cc23)C1c1ccc(OCCOCCOC)cc1] | 404 |
| 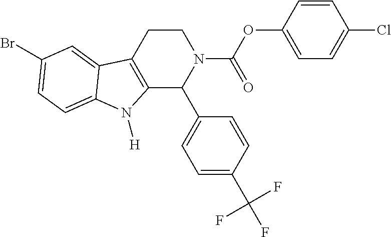 [SMILES: O=C(Oc1ccc(Cl)cc1)N1CCc2c([nH]c3ccc(Br)cc23)C1c1ccc(C(F)(F)F)cc1] | 405 |
| 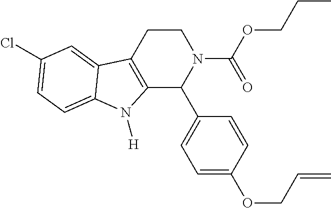 [SMILES: C=CCOc1ccc(C2c3[nH]c4ccc(Cl)cc4c3CCN2C(=O)OCCC)cc1] | 406 |
| 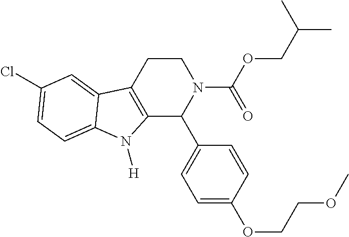 [SMILES: COCCOc1ccc(C2c3[nH]c4ccc(Cl)cc4c3CCN2C(=O)OCC(C)C)cc1] | 407 |
| 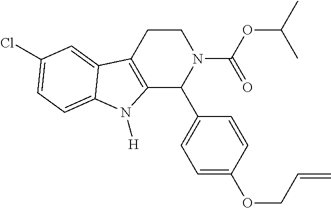 [SMILES: C=CCOc1ccc(C2c3[nH]c4ccc(Cl)cc4c3CCN2C(=O)OC(C)C)cc1] | 408 |
| 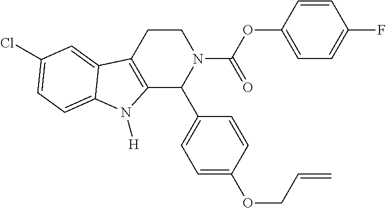 [SMILES: C=CCOc1ccc(C2c3[nH]c4ccc(Cl)cc4c3CCN2C(=O)Oc2ccc(F)cc2)cc1] | 409 |
| 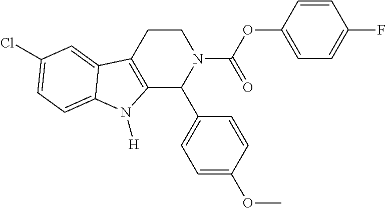 [SMILES: COc1ccc(C2c3[nH]c4ccc(Cl)cc4c3CCN2C(=O)Oc2ccc(F)cc2)cc1] | 410 |
| 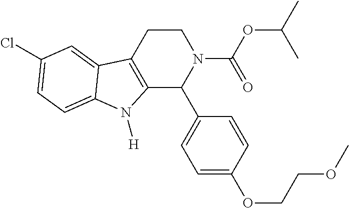 [SMILES: COCCOc1ccc(C2c3[nH]c4ccc(Cl)cc4c3CCN2C(=O)OC(C)C)cc1] | 411 |
| 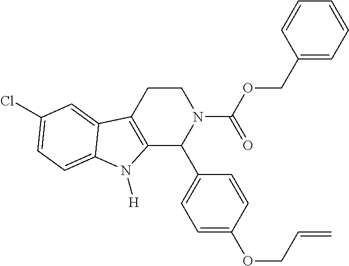 [SMILES: C=CCOc1ccc(C2c3[nH]c4ccc(Cl)cc4c3CCN2C(=O)OCc2ccccc2)cc1] | 412 |
| 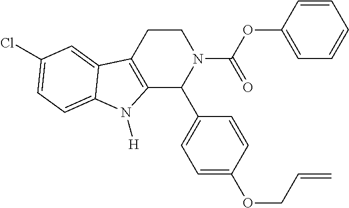 [SMILES: C=CCOc1ccc(C2c3[nH]c4ccc(Cl)cc4c3CCN2C(=O)Oc2ccccc2)cc1] | 413 |
| 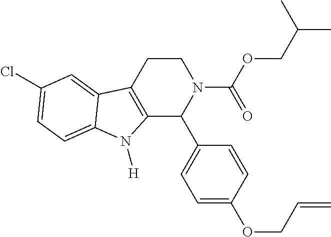 [SMILES: C=CCOc1ccc(C2c3[nH]c4ccc(Cl)cc4c3CCN2C(=O)OCC(C)C)cc1] | 414 |
| 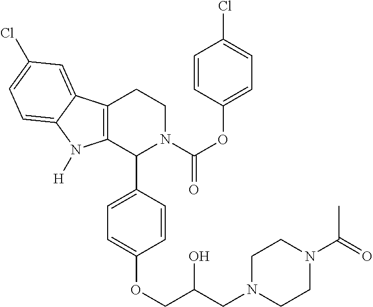 [SMILES: CC(=O)N1CCN(CC(O)COc2ccc(C3c4[nH]c5ccc(Cl)cc5c4CCN3C(=O)Oc3ccc(Cl)cc3)cc2)CC1] | 415 |
| 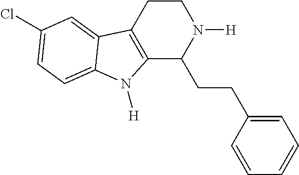 [SMILES: Clc1ccc2[nH]c3c(c2c1)CCNC3CCc1ccccc1] | 416 |
| 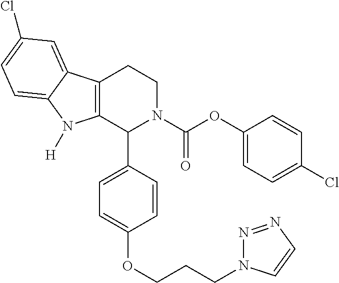 [SMILES: O=C(Oc1ccc(Cl)cc1)N1CCc2c([nH]c3ccc(Cl)cc23)C1c1ccc(OCCCn2ccnn2)cc1] | 417 |
| 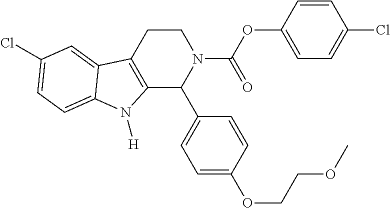 [SMILES: COCCOc1ccc(C2c3[nH]c4ccc(Cl)cc4c3CCN2C(=O)Oc2ccc(Cl)cc2)cc1] | 418 |
| 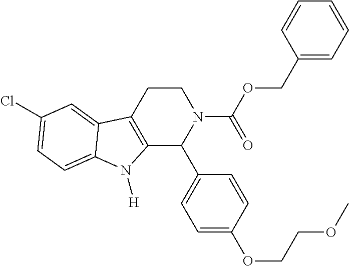 [SMILES: COCCOc1ccc(C2c3[nH]c4ccc(Cl)cc4c3CCN2C(=O)OCc2ccccc2)cc1] | 419 |
| 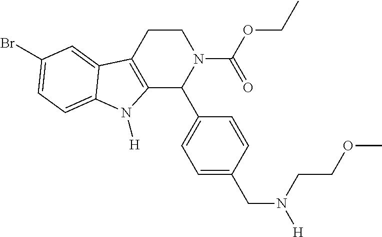 [SMILES: CCOC(=O)N1CCc2c([nH]c3ccc(Br)cc23)C1c1ccc(CNCCOC)cc1] | 420 |
| 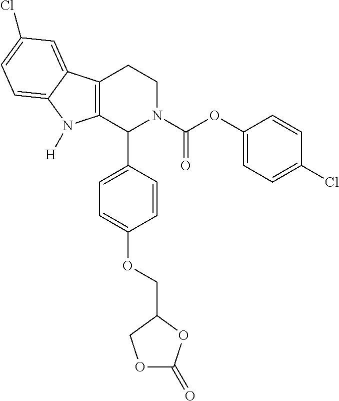 [SMILES: O=C1OCC(COc2ccc(C3c4[nH]c5ccc(Cl)cc5c4CCN3C(=O)Oc3ccc(Cl)cc3)cc2)O1] | 421 |
| 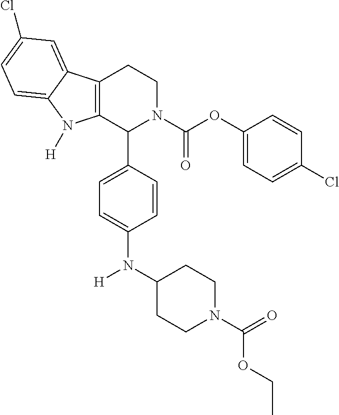 [SMILES: CCOC(=O)N1CCC(Nc2ccc(C3c4[nH]c5ccc(Cl)cc5c4CCN3C(=O)Oc3ccc(Cl)cc3)cc2)CC1] | 422 |
| 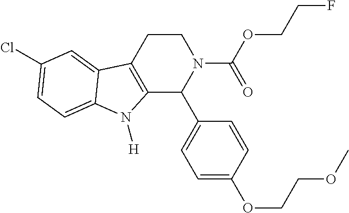 [SMILES: COCCOc1ccc(C2c3[nH]c4ccc(Cl)cc4c3CCN2C(=O)OCCF)cc1] | 423 |
| 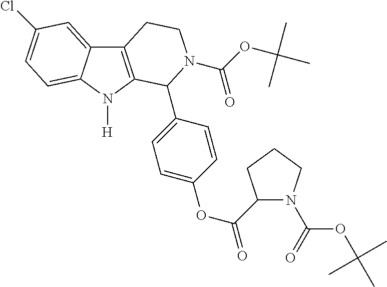 [SMILES: CC(C)(C)OC(=O)N1CCCC1C(=O)Oc1ccc(C2c3[nH]c4ccc(Cl)cc4c3CCN2C(=O)OC(C)(C)C)cc1] | 424 |
| 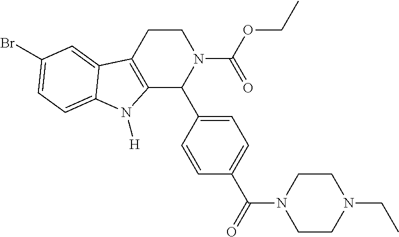 [SMILES: CCOC(=O)N1CCc2c([nH]c3ccc(Br)cc23)C1c1ccc(C(=O)N2CCN(CC)CC2)cc1] | 425 |
| 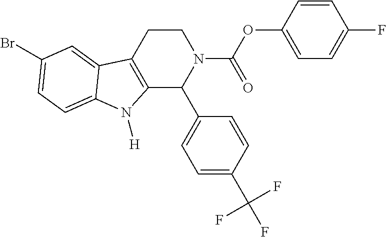 [SMILES: O=C(Oc1ccc(F)cc1)N1CCc2c([nH]c3ccc(Br)cc23)C1c1ccc(C(F)(F)F)cc1] | 426 |
| 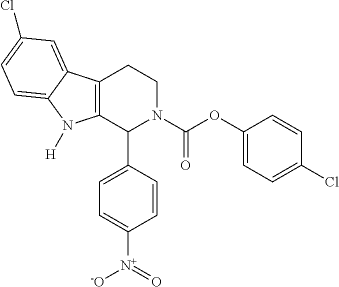 | 427 |
| 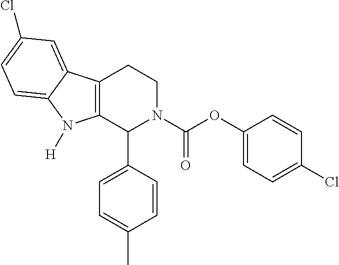 [SMILES: Cc1ccc(C2c3[nH]c4ccc(Cl)cc4c3CCN2C(=O)Oc2ccc(Cl)cc2)cc1] | 428 |
| 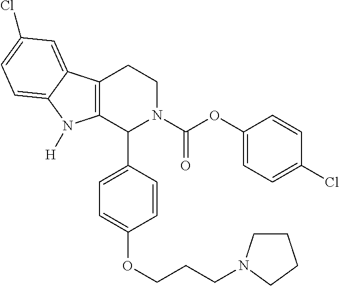 [SMILES: O=C(Oc1ccc(Cl)cc1)N1CCc2c([nH]c3ccc(Cl)cc23)C1c1ccc(OCCCN2CCCC2)cc1] | 429 |
| 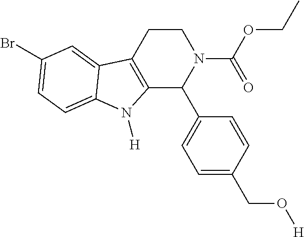 [SMILES: CCOC(=O)N1CCc2c([nH]c3ccc(Br)cc23)C1c1ccc(CO)cc1] | 430 |
| 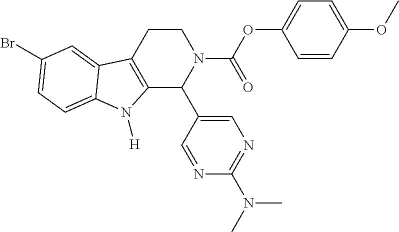 [SMILES: COc1ccc(OC(=O)N2CCc3c([nH]c4ccc(Br)cc34)C2c2cnc(N(C)C)nc2)cc1] | 431 |
| 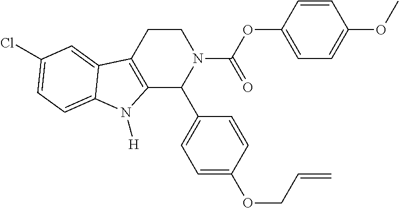 [SMILES: C=CCOc1ccc(C2c3[nH]c4ccc(Cl)cc4c3CCN2C(=O)Oc2ccc(OC)cc2)cc1] | 432 |
| 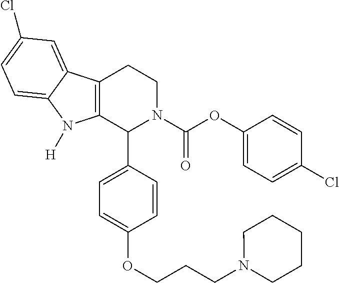 [SMILES: O=C(Oc1ccc(Cl)cc1)N1CCc2c([nH]c3ccc(Cl)cc23)C1c1ccc(OCCCN2CCCCC2)cc1] | 433 |
| 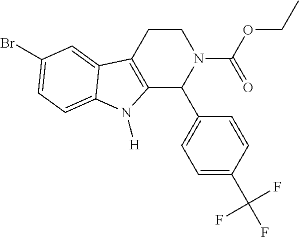 [SMILES: CCOC(=O)N1CCc2c([nH]c3ccc(Br)cc23)C1c1ccc(C(F)(F)F)cc1] | 434 |
| 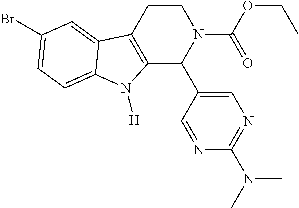 [SMILES: CCOC(=O)N1CCc2c([nH]c3ccc(Br)cc23)C1c1cnc(N(C)C)nc1] | 435 |
| 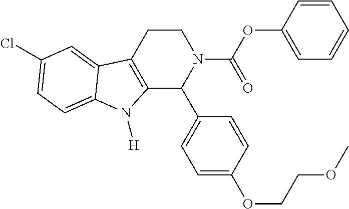 [SMILES: COCCOc1ccc(C2c3[nH]c4ccc(Cl)cc4c3CCN2C(=O)Oc2ccccc2)cc1] | 436 |
| 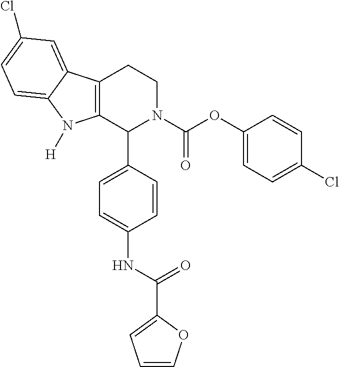 [SMILES: O=C(Nc1ccc(C2c3[nH]c4ccc(Cl)cc4c3CCN2C(=O)Oc2ccc(Cl)cc2)cc1)c1ccco1] | 437 |
| 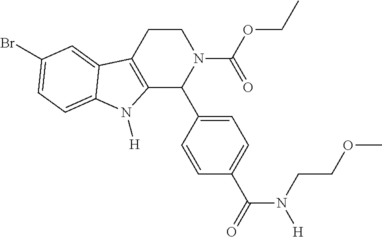 [SMILES: CCOC(=O)N1CCc2c([nH]c3ccc(Br)cc23)C1c1ccc(C(=O)NCCOC)cc1] | 438 |
| 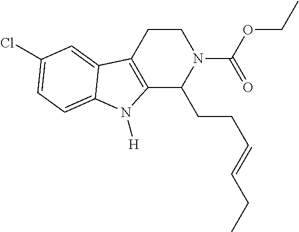 [SMILES: CC/C=C/CCC1c2[nH]c3ccc(Cl)cc3c2CCN1C(=O)OCC] | 439 |
| 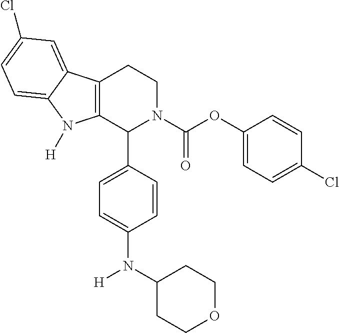 [SMILES: O=C(Oc1ccc(Cl)cc1)N1CCc2c([nH]c3ccc(Cl)cc23)C1c1ccc(NC2CCOCC2)cc1] | 440 |
| 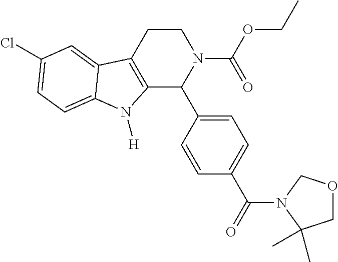 [SMILES: CCOC(=O)N1CCc2c([nH]c3ccc(Cl)cc23)C1c1ccc(C(=O)N2COCC2(C)C)cc1] | 441 |
| 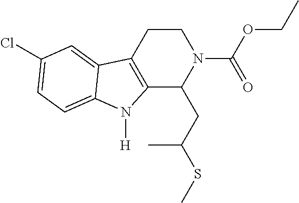 [SMILES: CCOC(=O)N1CCc2c([nH]c3ccc(Cl)cc23)C1CC(C)SC] | 442 |
| 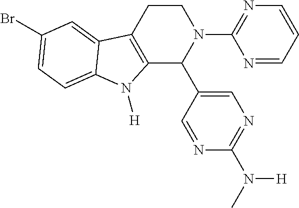 [SMILES: CNc1ncc(C2c3[nH]c4ccc(Br)cc4c3CCN2c2ncccn2)cn1] | 443 |
| 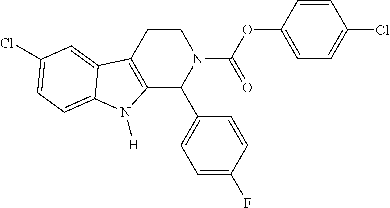 [SMILES: O=C(Oc1ccc(Cl)cc1)N1CCc2c([nH]c3ccc(Cl)cc23)C1c1ccc(F)cc1] | 444 |
| 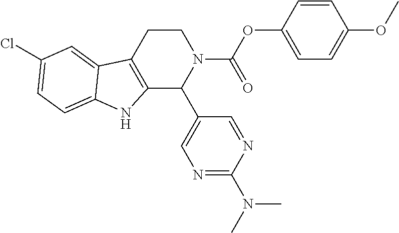 [SMILES: COc1ccc(OC(=O)N2CCc3c([nH]c4ccc(Cl)cc34)C2c2cnc(N(C)C)nc2)cc1] | 445 |
| 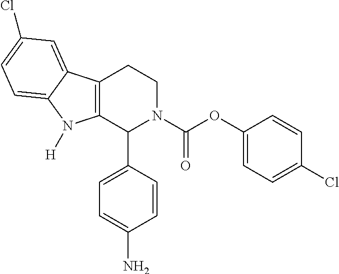 [SMILES: Nc1ccc(C2c3[nH]c4ccc(Cl)cc4c3CCN2C(=O)Oc2ccc(Cl)cc2)cc1] | 446 |
| 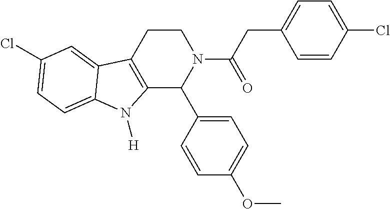 [SMILES: COc1ccc(C2c3[nH]c4ccc(Cl)cc4c3CCN2C(=O)Cc2ccc(Cl)cc2)cc1] | 447 |
| 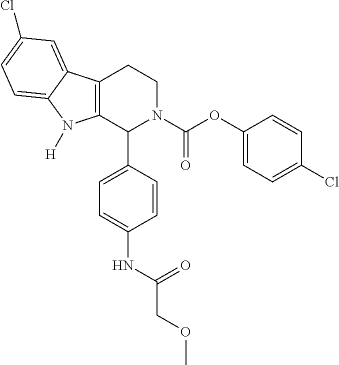 [SMILES: COCC(=O)Nc1ccc(C2c3[nH]c4ccc(Cl)cc4c3CCN2C(=O)Oc2ccc(Cl)cc2)cc1] | 448 |
| 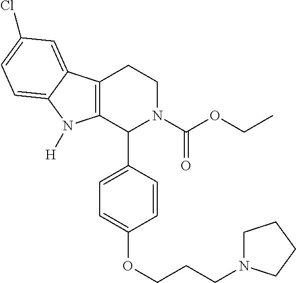 [SMILES: CCOC(=O)N1CCc2c([nH]c3ccc(Cl)cc23)C1c1ccc(OCCCN2CCCC2)cc1] | 449 |
| 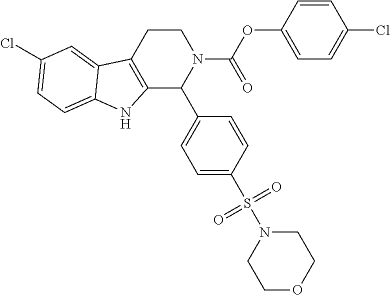 [SMILES: O=C(Oc1ccc(Cl)cc1)N1CCc2c([nH]c3ccc(Cl)cc23)C1c1ccc(S(=O)(=O)N2CCOCC2)cc1] | 450 |
| 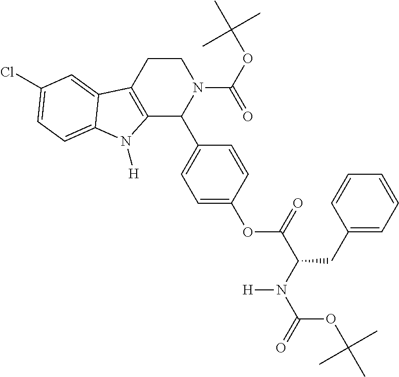 [SMILES: CC(C)(C)OC(=O)N[C@@H](Cc1ccccc1)C(=O)Oc1ccc(C2c3[nH]c4ccc(Cl)cc4c3CCN2C(=O)OC(C)(C)C)cc1] | 451 |
| 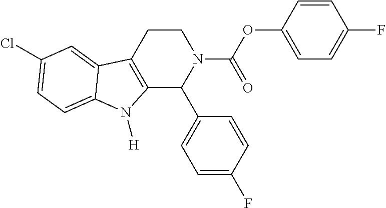 [SMILES: O=C(Oc1ccc(F)cc1)N1CCc2c([nH]c3ccc(Cl)cc23)C1c1ccc(F)cc1] | 452 |
| 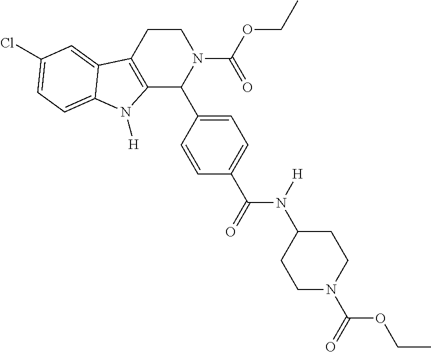 [SMILES: CCOC(=O)N1CCC(NC(=O)c2ccc(C3c4[nH]c5ccc(Cl)cc5c4CCN3C(=O)OCC)cc2)CC1] | 453 |
| 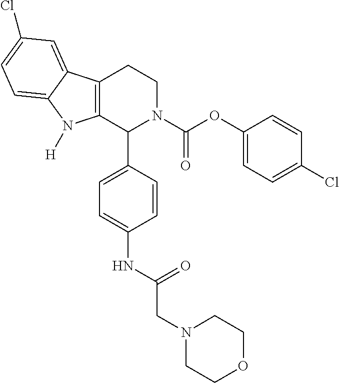 [SMILES: O=C(CN1CCOCC1)Nc1ccc(C2c3[nH]c4ccc(Cl)cc4c3CCN2C(=O)Oc2ccc(Cl)cc2)cc1] | 454 |
| 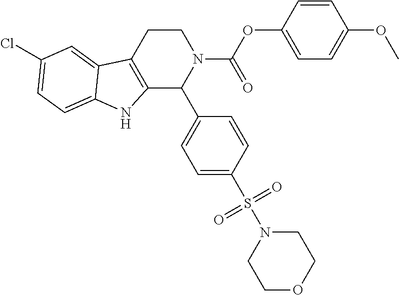 [SMILES: COc1ccc(OC(=O)N2CCc3c([nH]c4ccc(Cl)cc34)C2c2ccc(S(=O)(=O)N3CCOCC3)cc2)cc1] | 455 |
| 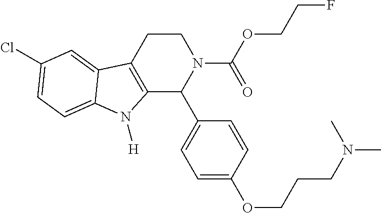 [SMILES: CN(C)CCCOc1ccc(C2c3[nH]c4ccc(Cl)cc4c3CCN2C(=O)OCCF)cc1] | 456 |
| 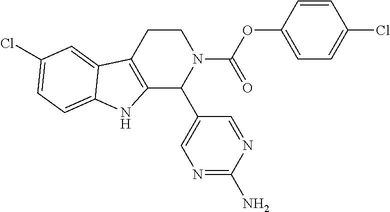 [SMILES: Nc1ncc(C2c3[nH]c4ccc(Cl)cc4c3CCN2C(=O)Oc2ccc(Cl)cc2)cn1] | 457 |
| 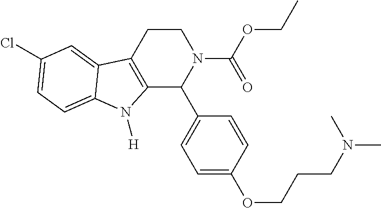 [SMILES: CCOC(=O)N1CCc2c([nH]c3ccc(Cl)cc23)C1c1ccc(OCCCN(C)C)cc1] | 458 |
| 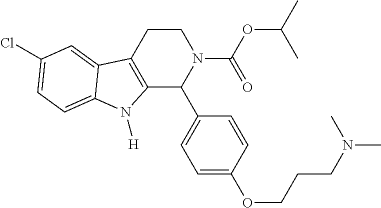 [SMILES: CC(C)OC(=O)N1CCc2c([nH]c3ccc(Cl)cc23)C1c1ccc(OCCCN(C)C)cc1] | 459 |
| 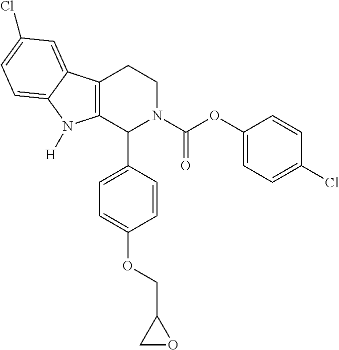 [SMILES: O=C(Oc1ccc(Cl)cc1)N1CCc2c([nH]c3ccc(Cl)cc23)C1c1ccc(OCC2CO2)cc1] | 460 |
| 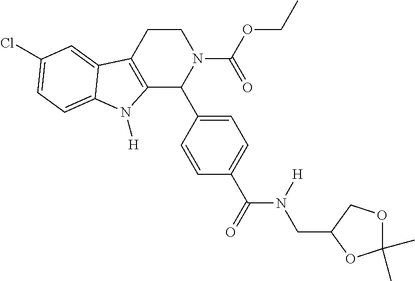 [SMILES: CCOC(=O)N1CCc2c([nH]c3ccc(Cl)cc23)C1c1ccc(C(=O)NCC2COC(C)(C)O2)cc1] | 461 |
| 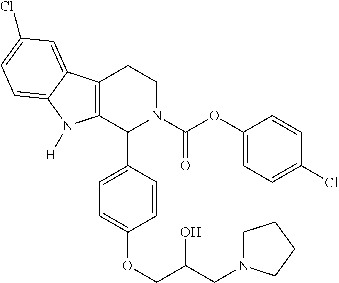 [SMILES: O=C(Oc1ccc(Cl)cc1)N1CCc2c([nH]c3ccc(Cl)cc23)C1c1ccc(OCC(O)CN2CCCC2)cc1] | 462 |
| 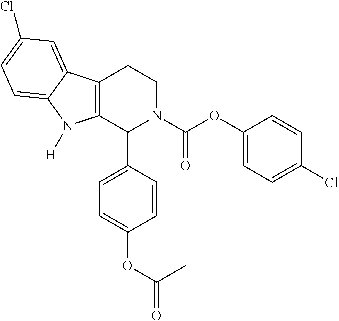 [SMILES: CC(=O)Oc1ccc(C2c3[nH]c4ccc(Cl)cc4c3CCN2C(=O)Oc2ccc(Cl)cc2)cc1] | 463 |
| 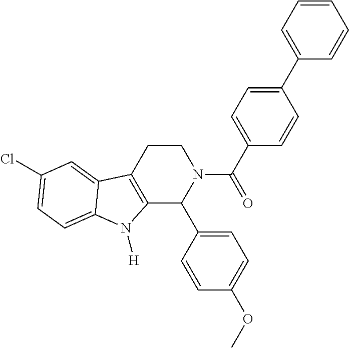 [SMILES: COc1ccc(C2c3[nH]c4ccc(Cl)cc4c3CCN2C(=O)c2ccc(-c3ccccc3)cc2)cc1] | 464 |
| 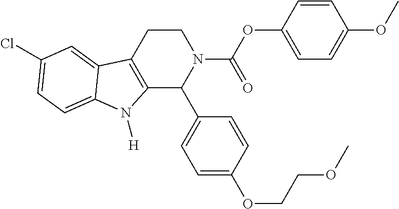 [SMILES: COCCOc1ccc(C2c3[nH]c4ccc(Cl)cc4c3CCN2C(=O)Oc2ccc(OC)cc2)cc1] | 465 |
| 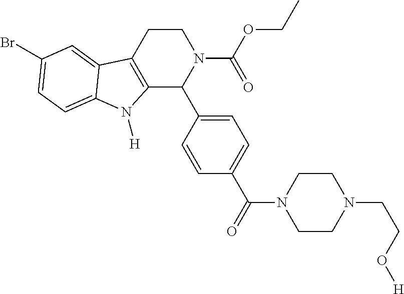 [SMILES: CCOC(=O)N1CCc2c([nH]c3ccc(Br)cc23)C1c1ccc(C(=O)N2CCN(CCO)CC2)cc1] | 466 |
| 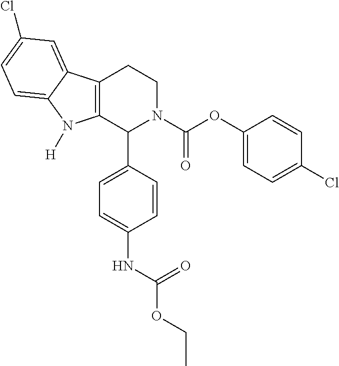 [SMILES: CCOC(=O)Nc1ccc(C2c3[nH]c4ccc(Cl)cc4c3CCN2C(=O)Oc2ccc(Cl)cc2)cc1] | 467 |
| 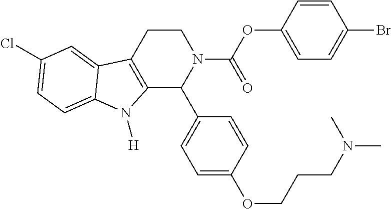 [SMILES: CN(C)CCCOc1ccc(C2c3[nH]c4ccc(Cl)cc4c3CCN2C(=O)Oc2ccc(Br)cc2)cc1] | 468 |
| 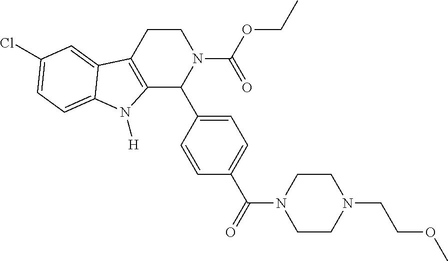 [SMILES: CCOC(=O)N1CCc2c([nH]c3ccc(Cl)cc23)C1c1ccc(C(=O)N2CCN(CCOC)CC2)cc1] | 469 |
| 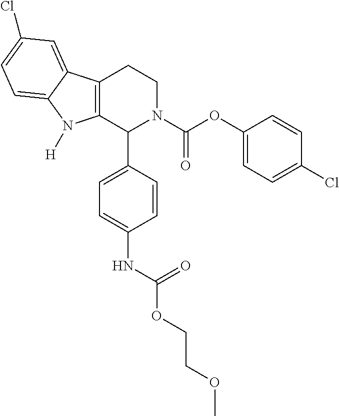 [SMILES: COCCOC(=O)Nc1ccc(C2c3[nH]c4ccc(Cl)cc4c3CCN2C(=O)Oc2ccc(Cl)cc2)cc1] | 470 |
| 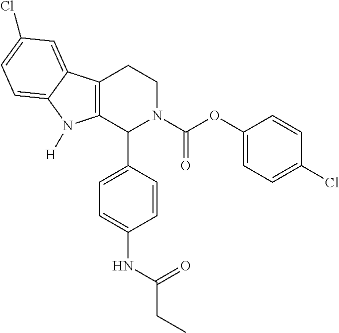 [SMILES: CCC(=O)Nc1ccc(C2c3[nH]c4ccc(Cl)cc4c3CCN2C(=O)Oc2ccc(Cl)cc2)cc1] | 471 |
| 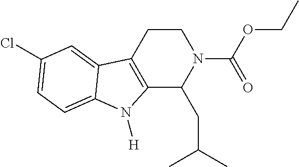 [SMILES: CCOC(=O)N1CCc2c([nH]c3ccc(Cl)cc23)C1CC(C)C] | 472 |
| 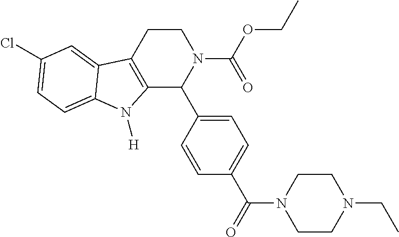 [SMILES: CCOC(=O)N1CCc2c([nH]c3ccc(Cl)cc23)C1c1ccc(C(=O)N2CCN(CC)CC2)cc1] | 473 |
| 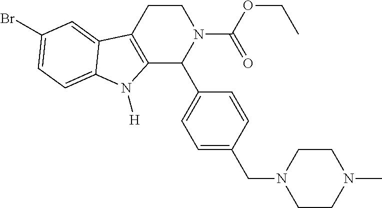 [SMILES: CCOC(=O)N1CCc2c([nH]c3ccc(Br)cc23)C1c1ccc(CN2CCN(C)CC2)cc1] | 474 |
| 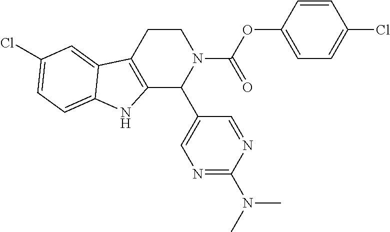 [SMILES: CN(C)c1ncc(C2c3[nH]c4ccc(Cl)cc4c3CCN2C(=O)Oc2ccc(Cl)cc2)cn1] | 475 |
| 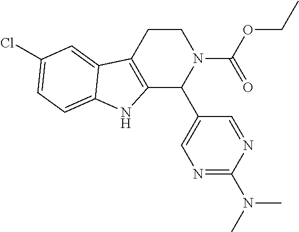 [SMILES: CCOC(=O)N1CCc2c([nH]c3ccc(Cl)cc23)C1c1cnc(N(C)C)nc1] | 476 |
| 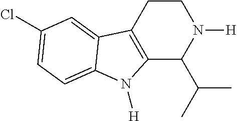 [SMILES: CC(C)C1NCCc2c1[nH]c1ccc(Cl)cc21] | 477 |
| 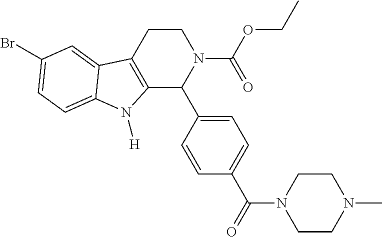 [SMILES: CCOC(=O)N1CCc2c([nH]c3ccc(Br)cc23)C1c1ccc(C(=O)N2CCN(C)CC2)cc1] | 478 |
| 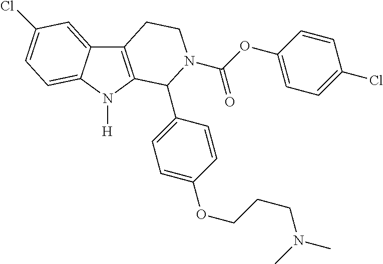 [SMILES: CN(C)CCCOc1ccc(C2c3[nH]c4ccc(Cl)cc4c3CCN2C(=O)Oc2ccc(Cl)cc2)cc1] | 479 |
| 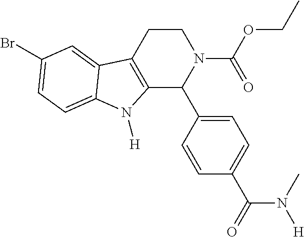 [SMILES: CCOC(=O)N1CCc2c([nH]c3ccc(Br)cc23)C1c1ccc(C(=O)NC)cc1] | 480 |
| 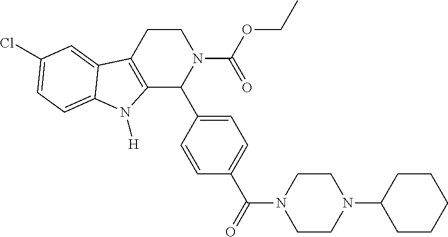 [SMILES: CCOC(=O)N1CCc2c([nH]c3ccc(Cl)cc23)C1c1ccc(C(=O)N2CCN(C3CCCCC3)CC2)cc1] | 481 |
| 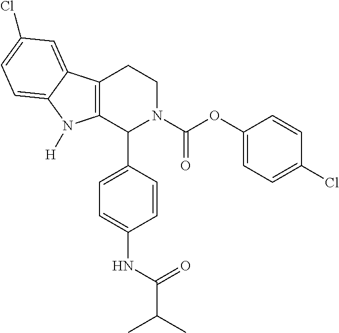 [SMILES: CC(C)C(=O)Nc1ccc(C2c3[nH]c4ccc(Cl)cc4c3CCN2C(=O)Oc2ccc(Cl)cc2)cc1] | 482 |
| 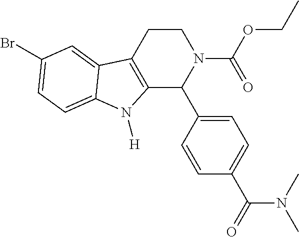 [SMILES: CCOC(=O)N1CCc2c([nH]c3ccc(Br)cc23)C1c1ccc(C(=O)N(C)C)cc1] | 483 |
| 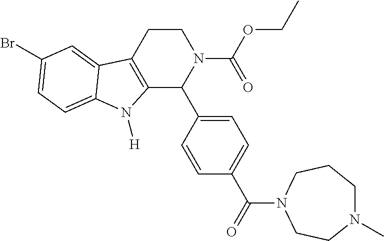 [SMILES: CCOC(=O)N1CCc2c([nH]c3ccc(Br)cc23)C1c1ccc(C(=O)N2CCCN(C)CC2)cc1] | 484 |
| 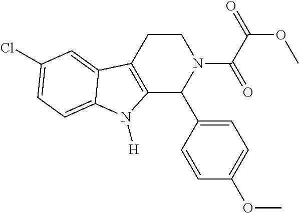 [SMILES: COC(=O)C(=O)N1CCc2c([nH]c3ccc(Cl)cc23)C1c1ccc(OC)cc1] | 485 |
| 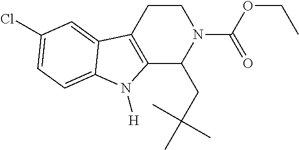 [SMILES: CCOC(=O)N1CCc2c([nH]c3ccc(Cl)cc23)C1CC(C)(C)C] | 486 |
| 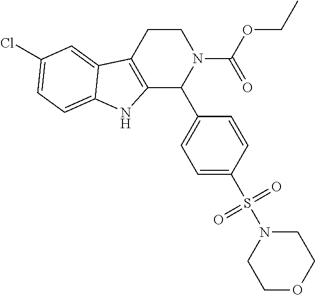 [SMILES: CCOC(=O)N1CCc2c([nH]c3ccc(Cl)cc23)C1c1ccc(S(=O)(=O)N2CCOCC2)cc1] | 487 |
| 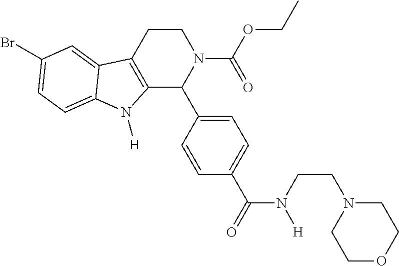 [SMILES: CCOC(=O)N1CCc2c([nH]c3ccc(Br)cc23)C1c1ccc(C(=O)NCCN2CCOCC2)cc1] | 488 |
| 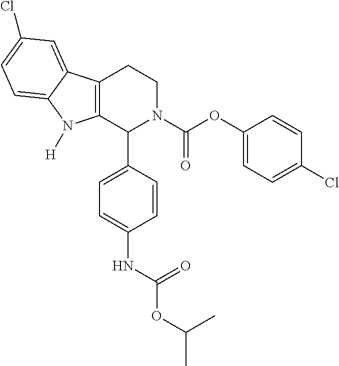 [SMILES: CC(C)OC(=O)Nc1ccc(C2c3[nH]c4ccc(Cl)cc4c3CCN2C(=O)Oc2ccc(Cl)cc2)cc1] | 489 |
| 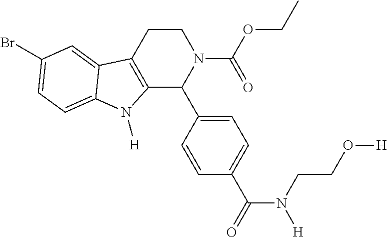 [SMILES: CCOC(=O)N1CCc2c([nH]c3ccc(Br)cc23)C1c1ccc(C(=O)NCCO)cc1] | 490 |
| 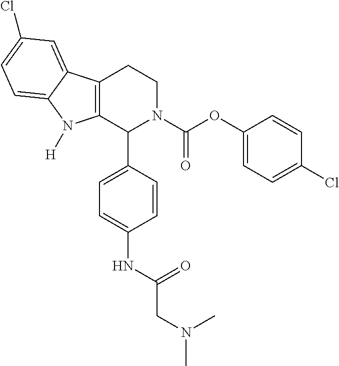 [SMILES: CN(C)CC(=O)Nc1ccc(C2c3[nH]c4ccc(Cl)cc4c3CCN2C(=O)Oc2ccc(Cl)cc2)cc1] | 491 |
| 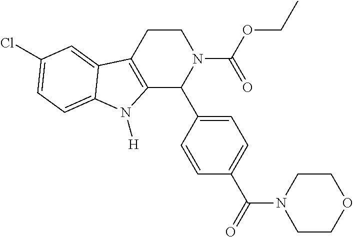 [SMILES: CCOC(=O)N1CCc2c([nH]c3ccc(Cl)cc23)C1c1ccc(C(=O)N2CCOCC2)cc1] | 492 |
| 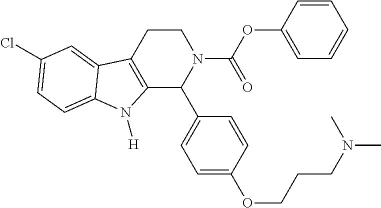 [SMILES: CN(C)CCCOc1ccc(C2c3[nH]c4ccc(Cl)cc4c3CCN2C(=O)Oc2ccccc2)cc1] | 493 |
| 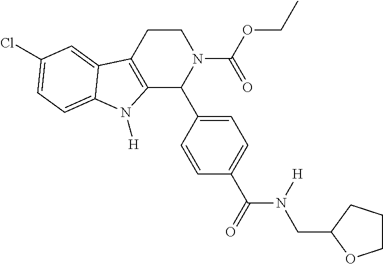 [SMILES: CCOC(=O)N1CCc2c([nH]c3ccc(Cl)cc23)C1c1ccc(C(=O)NCC2CCCO2)cc1] | 494 |
| 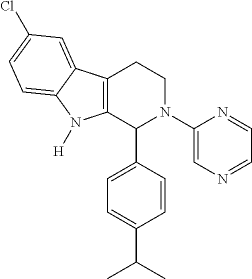 [SMILES: CC(C)c1ccc(C2c3[nH]c4ccc(Cl)cc4c3CCN2c2cnccn2)cc1] | 495 |
| 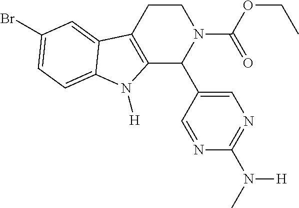 [SMILES: CCOC(=O)N1CCc2c([nH]c3ccc(Br)cc23)C1c1cnc(NC)nc1] | 496 |
| 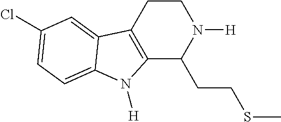 [SMILES: CSCCC1NCCc2c1[nH]c1ccc(Cl)cc21] | 497 |
| 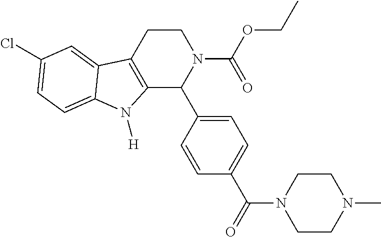 [SMILES: CCOC(=O)N1CCc2c([nH]c3ccc(Cl)cc23)C1c1ccc(C(=O)N2CCN(C)CC2)cc1] | 498 |
| 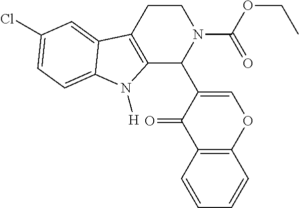 [SMILES: CCOC(=O)N1CCc2c([nH]c3ccc(Cl)cc23)C1c1coc2ccccc2c1=O] | 499 |
| 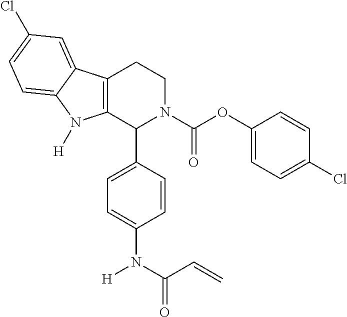 [SMILES: C=CC(=O)Nc1ccc(C2c3[nH]c4ccc(Cl)cc4c3CCN2C(=O)Oc2ccc(Cl)cc2)cc1] | 500 |
| 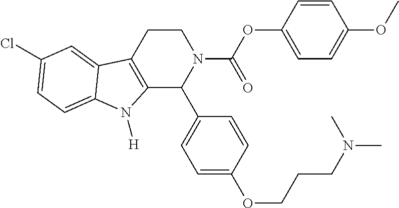 [SMILES: COc1ccc(OC(=O)N2CCc3c([nH]c4ccc(Cl)cc34)C2c2ccc(OCCCN(C)C)cc2)cc1] | 501 |
| 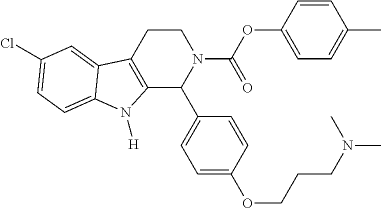 [SMILES: Cc1ccc(OC(=O)N2CCc3c([nH]c4ccc(Cl)cc34)C2c2ccc(OCCCN(C)C)cc2)cc1] | 502 |
| 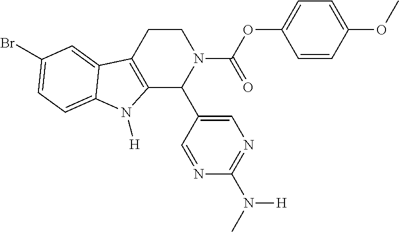 [SMILES: CNc1ncc(C2c3[nH]c4ccc(Br)cc4c3CCN2C(=O)Oc2ccc(OC)cc2)cn1] | 503 |
| 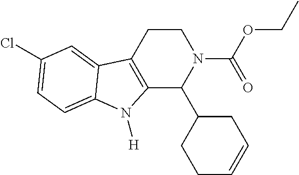 [SMILES: CCOC(=O)N1CCc2c([nH]c3ccc(Cl)cc23)C1C1CC=CCC1] | 504 |
| 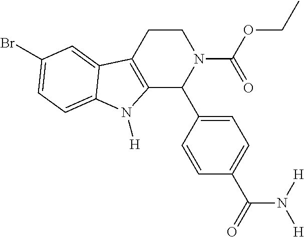 [SMILES: CCOC(=O)N1CCc2c([nH]c3ccc(Br)cc23)C1c1ccc(C(N)=O)cc1] | 505 |
| 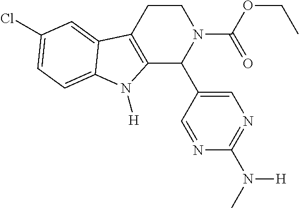 [SMILES: CCOC(=O)N1CCc2c([nH]c3ccc(Cl)cc23)C1c1cnc(NC)nc1] | 506 |
| 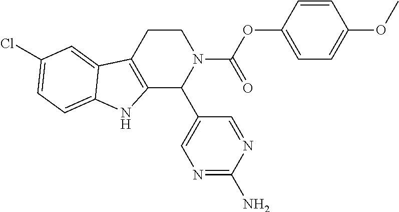 [SMILES: COc1ccc(OC(=O)N2CCc3c([nH]c4ccc(Cl)cc34)C2c2cnc(N)nc2)cc1] | 507 |
| 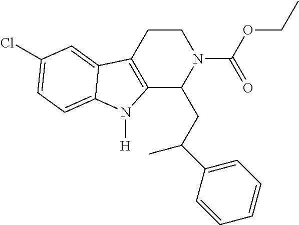 [SMILES: CCOC(=O)N1CCc2c([nH]c3ccc(Cl)cc23)C1CC(C)c1ccccc1] | 508 |
| 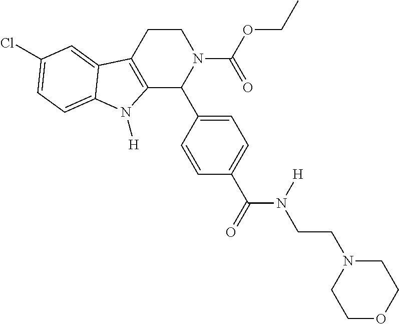 [SMILES: CCOC(=O)N1CCc2c([nH]c3ccc(Cl)cc23)C1c1ccc(C(=O)NCCN2CCOCC2)cc1] | 509 |
| 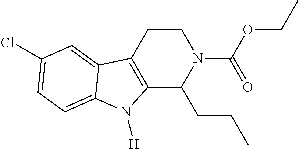 [SMILES: CCCC1c2[nH]c3ccc(Cl)cc3c2CCN1C(=O)OCC] | 510 |
| 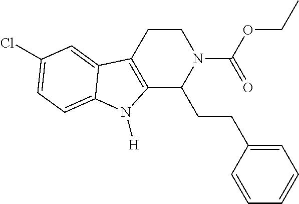 [SMILES: CCOC(=O)N1CCc2c([nH]c3ccc(Cl)cc23)C1CCc1ccccc1] | 511 |
| 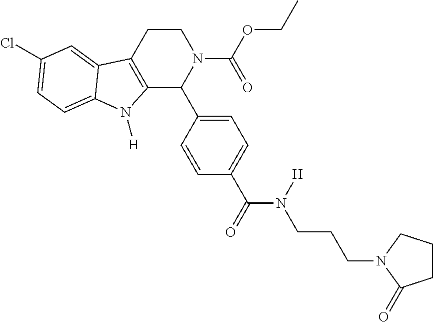 [SMILES: CCOC(=O)N1CCc2c([nH]c3ccc(Cl)cc23)C1c1ccc(C(=O)NCCCN2CCCC2=O)cc1] | 512 |
| 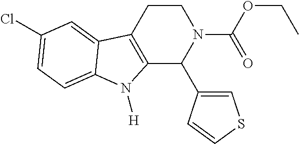 [SMILES: CCOC(=O)N1CCc2c([nH]c3ccc(Cl)cc23)C1c1ccsc1] | 513 |
| 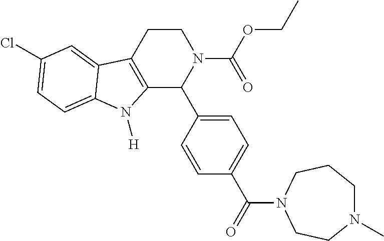 [SMILES: CCOC(=O)N1CCc2c([nH]c3ccc(Cl)cc23)C1c1ccc(C(=O)N2CCCN(C)CC2)cc1] | 514 |
| 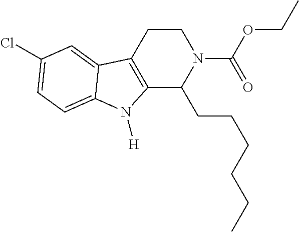 [SMILES: CCCCCCC1c2[nH]c3ccc(Cl)cc3c2CCN1C(=O)OCC] | 515 |
| 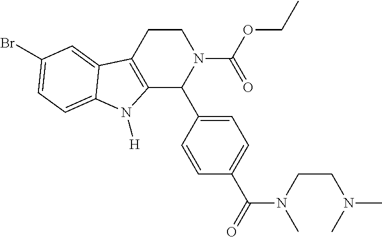 [SMILES: CCOC(=O)N1CCc2c([nH]c3ccc(Br)cc23)C1c1ccc(C(=O)N(C)CCN(C)C)cc1] | 516 |
| 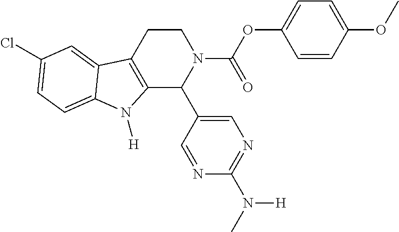 [SMILES: CNc1ncc(C2c3[nH]c4ccc(Cl)cc4c3CCN2C(=O)Oc2ccc(OC)cc2)cn1] | 517 |
| 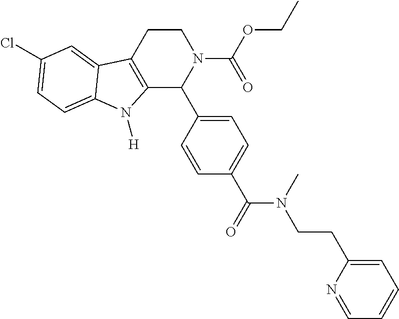 [SMILES: CCOC(=O)N1CCc2c([nH]c3ccc(Cl)cc23)C1c1ccc(C(=O)N(C)CCc2ccccn2)cc1] | 518 |
| 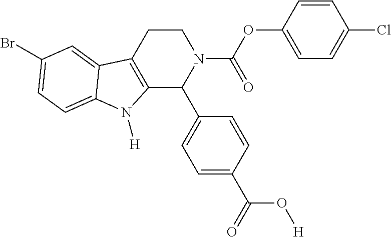 [SMILES: O=C(O)c1ccc(C2c3[nH]c4ccc(Br)cc4c3CCN2C(=O)Oc2ccc(Cl)cc2)cc1] | 519 |
| 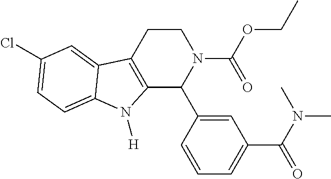 [SMILES: CCOC(=O)N1CCc2c([nH]c3ccc(Cl)cc23)C1c1cccc(C(=O)N(C)C)c1] | 520 |
| 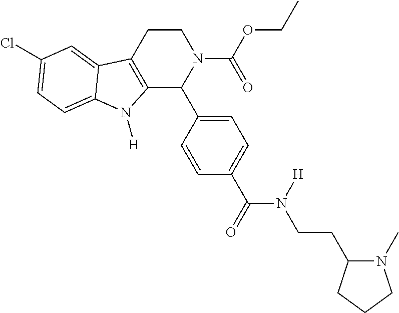 [SMILES: CCOC(=O)N1CCc2c([nH]c3ccc(Cl)cc23)C1c1ccc(C(=O)NCCC2CCCN2C)cc1] | 521 |
| 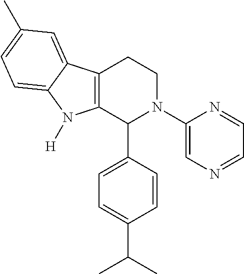 [SMILES: Cc1ccc2[nH]c3c(c2c1)CCN(c1cnccn1)C3c1ccc(C(C)C)cc1] | 522 |
| 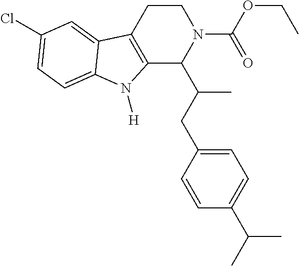 [SMILES: CCOC(=O)N1CCc2c([nH]c3ccc(Cl)cc23)C1C(C)Cc1ccc(C(C)C)cc1] | 523 |
| 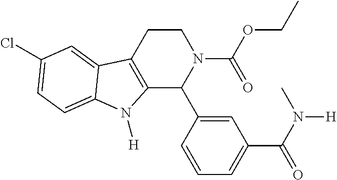 [SMILES: CCOC(=O)N1CCc2c([nH]c3ccc(Cl)cc23)C1c1cccc(C(=O)NC)c1] | 524 |
| 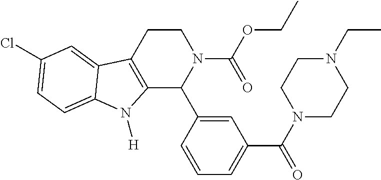 [SMILES: CCOC(=O)N1CCc2c([nH]c3ccc(Cl)cc23)C1c1cccc(C(=O)N2CCN(CC)CC2)c1] | 525 |
| 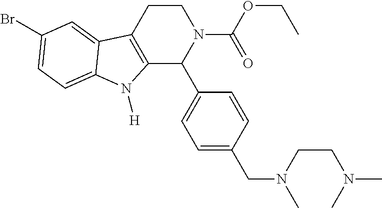 [SMILES: CCOC(=O)N1CCc2c([nH]c3ccc(Br)cc23)C1c1ccc(CN(C)CCN(C)C)cc1] | 526 |
| 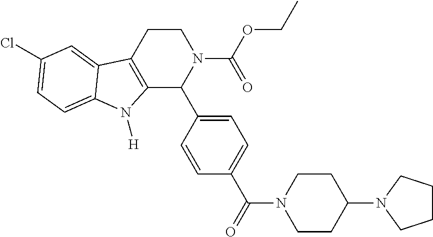 [SMILES: CCOC(=O)N1CCc2c([nH]c3ccc(Cl)cc23)C1c1ccc(C(=O)N2CCC(N3CCCC3)CC2)cc1] | 527 |
| 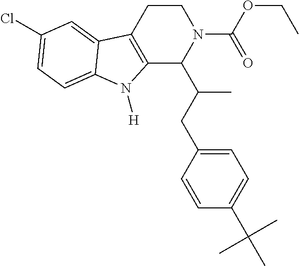 [SMILES: CCOC(=O)N1CCc2c([nH]c3ccc(Cl)cc23)C1C(C)Cc1ccc(C(C)(C)C)cc1] | 528 |
| 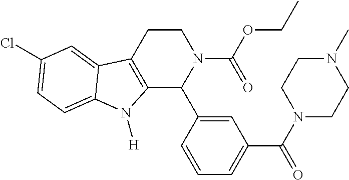 [SMILES: CCOC(=O)N1CCc2c([nH]c3ccc(Cl)cc23)C1c1cccc(C(=O)N2CCN(C)CC2)c1] | 529 |
| 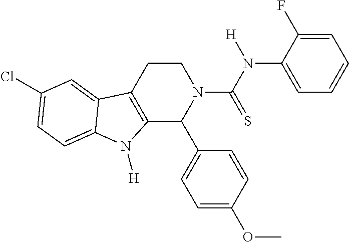 [SMILES: COc1ccc(C2c3[nH]c4ccc(Cl)cc4c3CCN2C(=S)Nc2ccccc2F)cc1] | 530 |
| 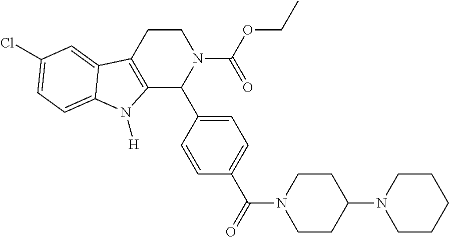 [SMILES: CCOC(=O)N1CCc2c([nH]c3ccc(Cl)cc23)C1c1ccc(C(=O)N2CCC(N3CCCCC3)CC2)cc1] | 531 |
| 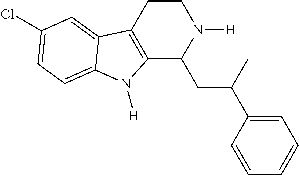 [SMILES: CC(CC1NCCc2c1[nH]c1ccc(Cl)cc21)c1ccccc1] | 532 |
| 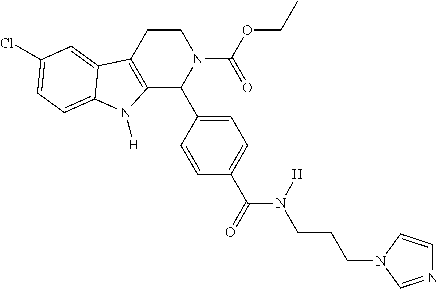 [SMILES: CCOC(=O)N1CCc2c([nH]c3ccc(Cl)cc23)C1c1ccc(C(=O)NCCCn2ccnc2)cc1] | 533 |
| 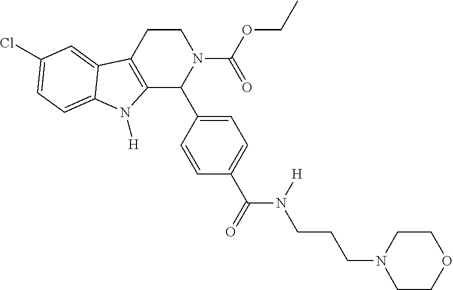 [SMILES: CCOC(=O)N1CCc2c([nH]c3ccc(Cl)cc23)C1c1ccc(C(=O)NCCCN2CCOCC2)cc1] | 534 |
| 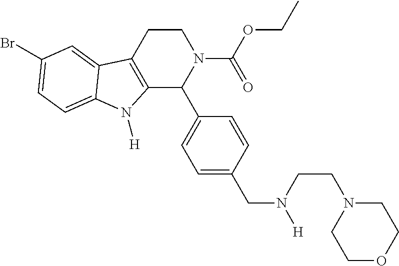 [SMILES: CCOC(=O)N1CCc2c([nH]c3ccc(Br)cc23)C1c1ccc(CNCCN2CCOCC2)cc1] | 535 |
| 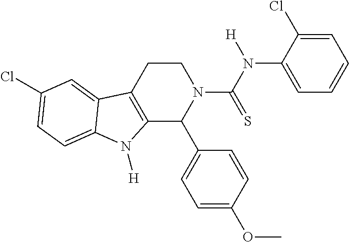 [SMILES: COc1ccc(C2c3[nH]c4ccc(Cl)cc4c3CCN2C(=S)Nc2ccccc2Cl)cc1] | 536 |
| 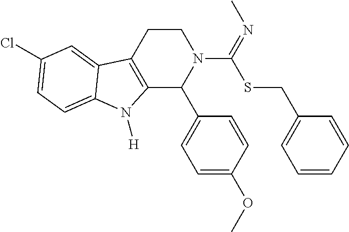 [SMILES: C/N=C(/SCc1ccccc1)N1CCc2c([nH]c3ccc(Cl)cc23)C1c1ccc(OC)cc1] | 537 |
| 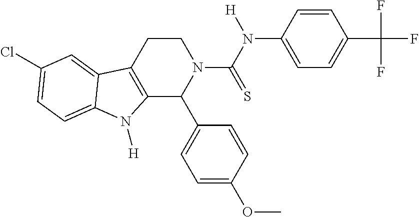 [SMILES: COc1ccc(C2c3[nH]c4ccc(Cl)cc4c3CCN2C(=S)Nc2ccc(C(F)(F)F)cc2)cc1] | 538 |
| 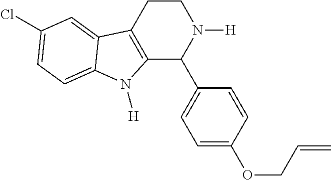 [SMILES: C=CCOc1ccc(C2NCCc3c2[nH]c2ccc(Cl)cc32)cc1] | 539 |
| 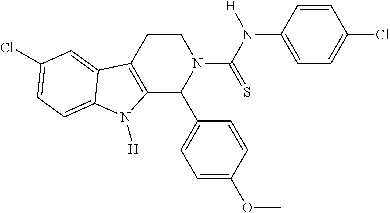 [SMILES: COc1ccc(C2c3[nH]c4ccc(Cl)cc4c3CCN2C(=S)Nc2ccc(Cl)cc2)cc1] | 540 |
| 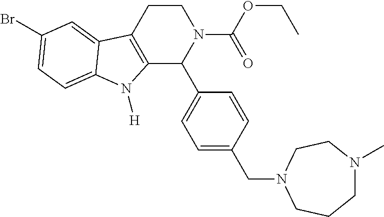 [SMILES: CCOC(=O)N1CCc2c([nH]c3ccc(Br)cc23)C1c1ccc(CN2CCCN(C)CC2)cc1] | 541 |
| 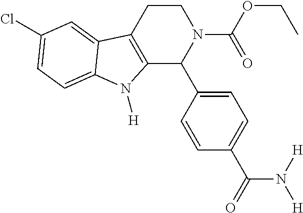 [SMILES: CCOC(=O)N1CCc2c([nH]c3ccc(Cl)cc23)C1c1ccc(C(N)=O)cc1] | 542 |
| 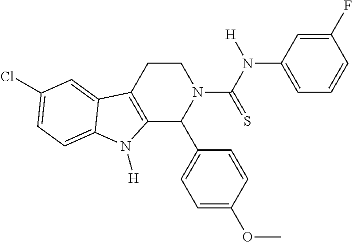 [SMILES: COc1ccc(C2c3[nH]c4ccc(Cl)cc4c3CCN2C(=S)Nc2cccc(F)c2)cc1] | 543 |
| 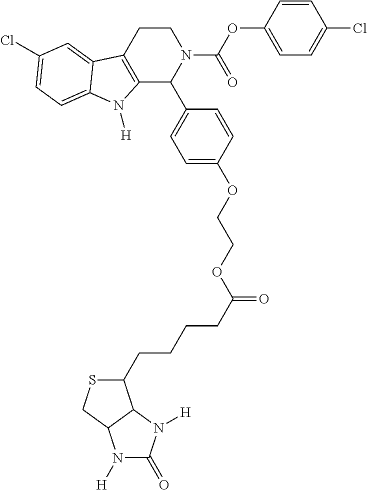 [SMILES: O=C1NC2CSC(CCCCC(=O)OCCOc3ccc(C4c5[nH]c6ccc(Cl)cc6c5CCN4C(=O)Oc4ccc(Cl)cc4)cc3)C2N1] | 544 |
| 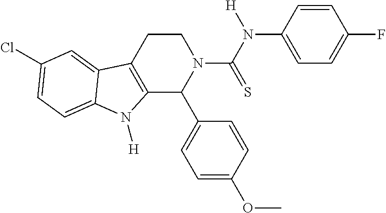 [SMILES: COc1ccc(C2c3[nH]c4ccc(Cl)cc4c3CCN2C(=S)Nc2ccc(F)cc2)cc1] | 545 |
| 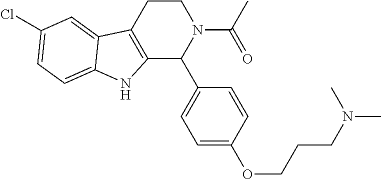 [SMILES: CC(=O)N1CCc2c([nH]c3ccc(Cl)cc23)C1c1ccc(OCCCN(C)C)cc1] | 546 |
| 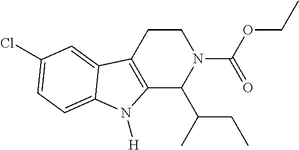 [SMILES: CCOC(=O)N1CCc2c([nH]c3ccc(Cl)cc23)C1C(C)CC] | 547 |
| 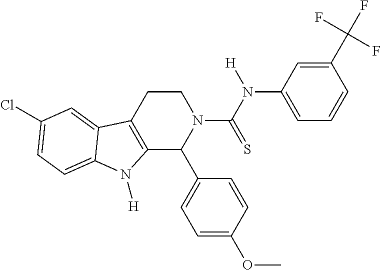 [SMILES: COc1ccc(C2c3[nH]c4ccc(Cl)cc4c3CCN2C(=S)Nc2cccc(C(F)(F)F)c2)cc1] | 548 |
| 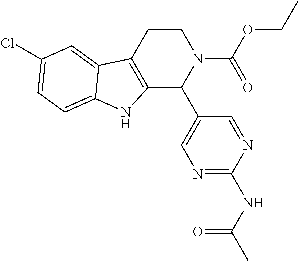 [SMILES: CCOC(=O)N1CCc2c([nH]c3ccc(Cl)cc23)C1c1cnc(NC(C)=O)nc1] | 549 |
| 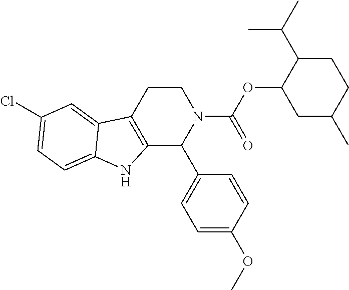 [SMILES: COc1ccc(C2c3[nH]c4ccc(Cl)cc4c3CCN2C(=O)OC2CC(C)CCC2C(C)C)cc1] | 550 |
| 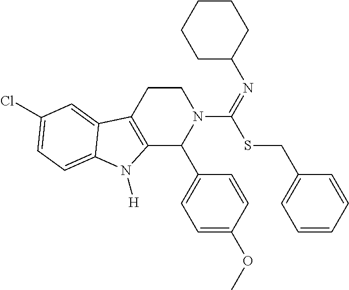 [SMILES: COc1ccc(C2c3[nH]c4ccc(Cl)cc4c3CCN2/C(=N\C2CCCCC2)SCc2ccccc2)cc1] | 551 |
| 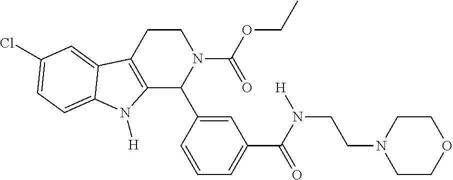 [SMILES: CCOC(=O)N1CCc2c([nH]c3ccc(Cl)cc23)C1c1cccc(C(=O)NCCN2CCOCC2)c1] | 552 |
| 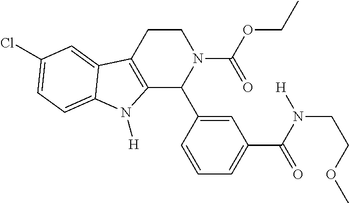 [SMILES: CCOC(=O)N1CCc2c([nH]c3ccc(Cl)cc23)C1c1cccc(C(=O)NCCOC)c1] | 553 |
| 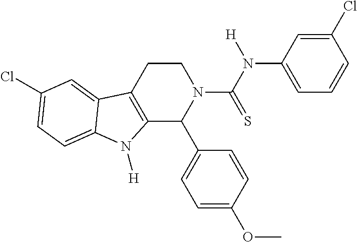 [SMILES: COc1ccc(C2c3[nH]c4ccc(Cl)cc4c3CCN2C(=S)Nc2cccc(Cl)c2)cc1] | 554 |
| 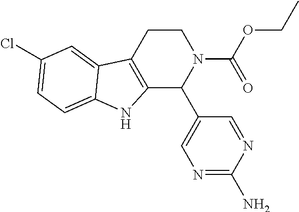 [SMILES: CCOC(=O)N1CCc2c([nH]c3ccc(Cl)cc23)C1c1cnc(N)nc1] | 555 |
| 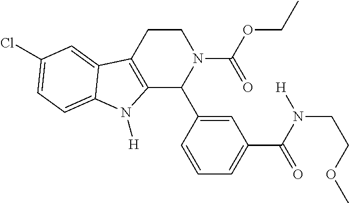 [SMILES: CCOC(=O)N1CCc2c([nH]c3ccc(Cl)cc23)C1c1cccc(C(=O)NCCOC)c1] | 556 |
| 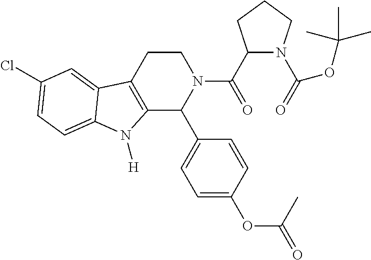 [SMILES: CC(=O)Oc1ccc(C2c3[nH]c4ccc(Cl)cc4c3CCN2C(=O)C2CCCN2C(=O)OC(C)(C)C)cc1] | 557 |
| 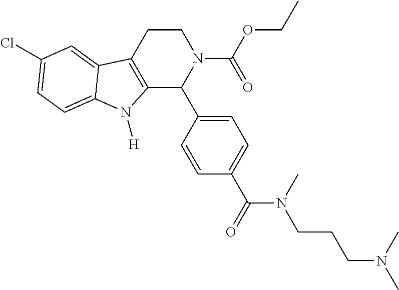 [SMILES: CCOC(=O)N1CCc2c([nH]c3ccc(Cl)cc23)C1c1ccc(C(=O)N(C)CCCN(C)C)cc1] | 558 |
| 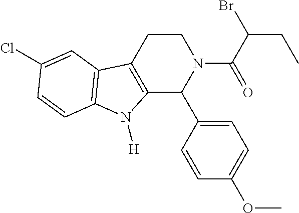 [SMILES: CCC(Br)C(=O)N1CCc2c([nH]c3ccc(Cl)cc23)C1c1ccc(OC)cc1] | 559 |
| 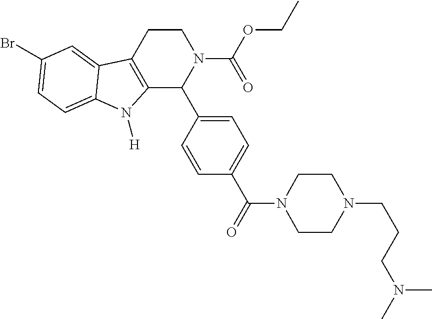 [SMILES: CCOC(=O)N1CCc2c([nH]c3ccc(Br)cc23)C1c1ccc(C(=O)N2CCN(CCCN(C)C)CC2)cc1] | 560 |
| 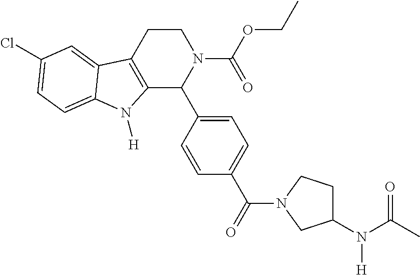 [SMILES: CCOC(=O)N1CCc2c([nH]c3ccc(Cl)cc23)C1c1ccc(C(=O)N2CCC(NC(C)=O)C2)cc1] | 561 |
| 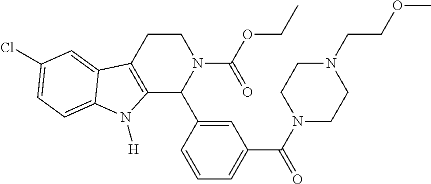 [SMILES: CCOC(=O)N1CCc2c([nH]c3ccc(Cl)cc23)C1c1cccc(C(=O)N2CCN(CCOC)CC2)c1] | 562 |
| 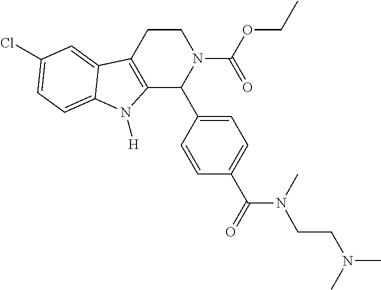 [SMILES: CCOC(=O)N1CCc2c([nH]c3ccc(Cl)cc23)C1c1ccc(C(=O)N(C)CCN(C)C)cc1] | 563 |
| 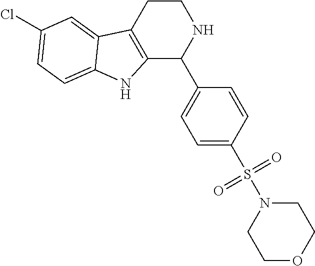 [SMILES: O=S(=O)(c1ccc(C2NCCc3c2[nH]c2ccc(Cl)cc32)cc1)N1CCOCC1] | 564 |
| 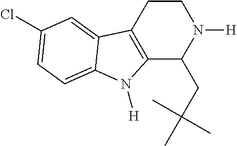 [SMILES: CC(C)(C)CC1NCCc2c1[nH]c1ccc(Cl)cc21] | 565 |
| 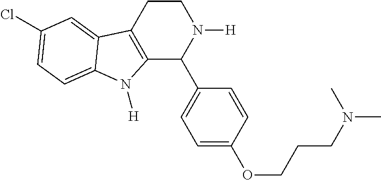 [SMILES: CN(C)CCCOc1ccc(C2NCCc3c2[nH]c2ccc(Cl)cc32)cc1] | 566 |
| 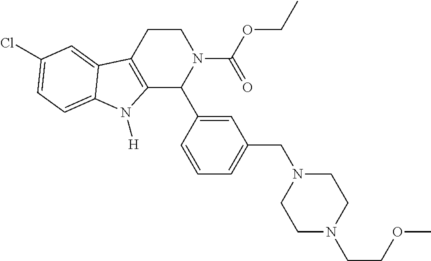 [SMILES: CCOC(=O)N1CCc2c([nH]c3ccc(Cl)cc23)C1c1cccc(CN2CCN(CCOC)CC2)c1] | 567 |
| 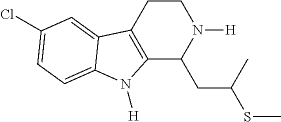 [SMILES: CSC(C)CC1NCCc2c1[nH]c1ccc(Cl)cc21] | 568 |
| 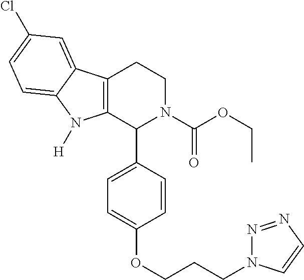 [SMILES: CCOC(=O)N1CCc2c([nH]c3ccc(Cl)cc23)C1c1ccc(OCCCn2ccnn2)cc1] | 569 |
| 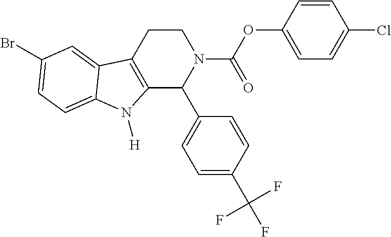 [SMILES: O=C(Oc1ccc(Cl)cc1)N1CCc2c([nH]c3ccc(Br)cc23)C1c1ccc(C(F)(F)F)cc1] | 570 |
| 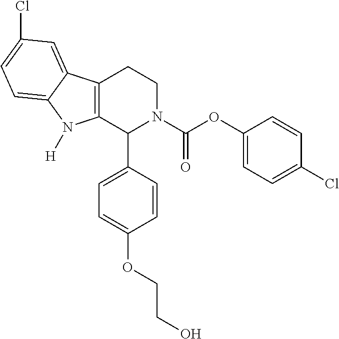 [SMILES: O=C(Oc1ccc(Cl)cc1)N1CCc2c([nH]c3ccc(Cl)cc23)C1c1ccc(OCCO)cc1] | 571 |
| 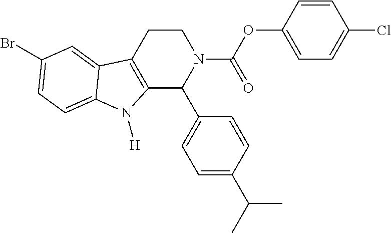 [SMILES: CC(C)c1ccc(C2c3[nH]c4ccc(Br)cc4c3CCN2C(=O)Oc2ccc(Cl)cc2)cc1] | 572 |
| 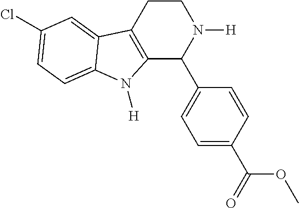 [SMILES: COC(=O)c1ccc(C2NCCc3c2[nH]c2ccc(Cl)cc32)cc1] | 573 |
| 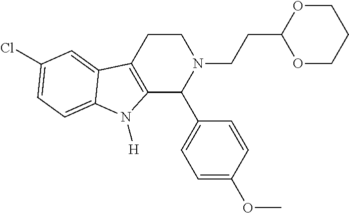 [SMILES: COc1ccc(C2c3[nH]c4ccc(Cl)cc4c3CCN2CCC2OCCCO2)cc1] | 574 |
| 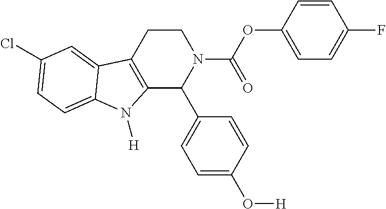 [SMILES: O=C(Oc1ccc(F)cc1)N1CCc2c([nH]c3ccc(Cl)cc23)C1c1ccc(O)cc1] | 575 |
| 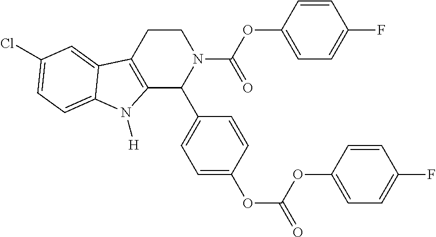 [SMILES: O=C(Oc1ccc(F)cc1)Oc1ccc(C2c3[nH]c4ccc(Cl)cc4c3CCN2C(=O)Oc2ccc(F)cc2)cc1] | 576 |
| 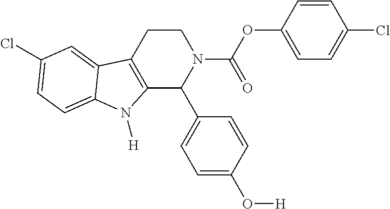 [SMILES: O=C(Oc1ccc(Cl)cc1)N1CCc2c([nH]c3ccc(Cl)cc23)C1c1ccc(O)cc1] | 577 |
| 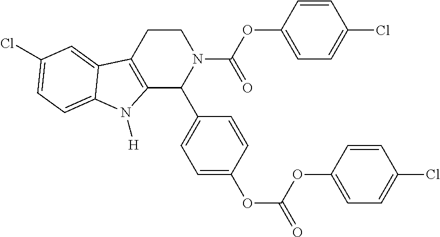 [SMILES: O=C(Oc1ccc(Cl)cc1)Oc1ccc(C2c3[nH]c4ccc(Cl)cc4c3CCN2C(=O)Oc2ccc(Cl)cc2)cc1] | 578 |
| 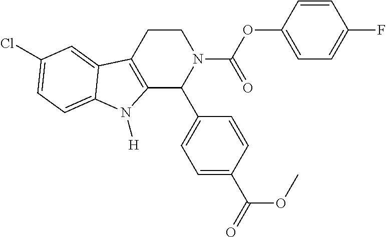 [SMILES: COC(=O)c1ccc(C2c3[nH]c4ccc(Cl)cc4c3CCN2C(=O)Oc2ccc(F)cc2)cc1] | 579 |
| 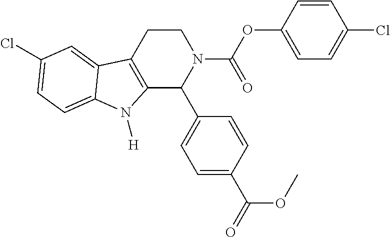 [SMILES: COC(=O)c1ccc(C2c3[nH]c4ccc(Cl)cc4c3CCN2C(=O)Oc2ccc(Cl)cc2)cc1] | 580 |
| 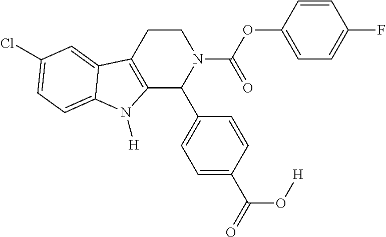 [SMILES: O=C(O)c1ccc(C2c3[nH]c4ccc(Cl)cc4c3CCN2C(=O)Oc2ccc(F)cc2)cc1] | 581 |
| 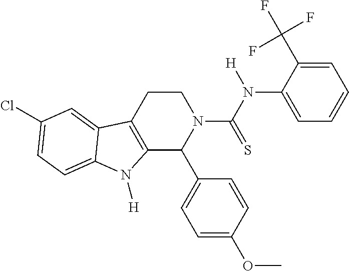 [SMILES: COc1ccc(C2c3[nH]c4ccc(Cl)cc4c3CCN2C(=S)Nc2ccccc2C(F)(F)F)cc1] | 582 |
| 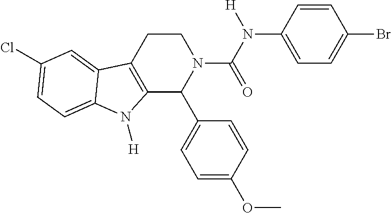 [SMILES: COc1ccc(C2c3[nH]c4ccc(Cl)cc4c3CCN2C(=O)Nc2ccc(Br)cc2)cc1] | 583 |
| 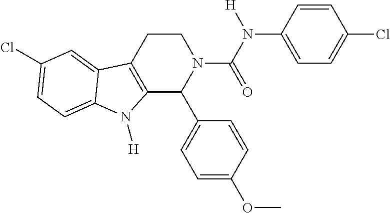 [SMILES: COc1ccc(C2c3[nH]c4ccc(Cl)cc4c3CCN2C(=O)Nc2ccc(Cl)cc2)cc1] | 584 |
| 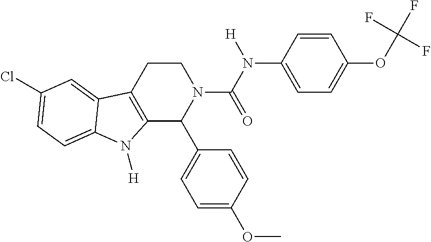 [SMILES: COc1ccc(C2c3[nH]c4ccc(Cl)cc4c3CCN2C(=O)Nc2ccc(OC(F)(F)F)cc2)cc1] | 585 |
| 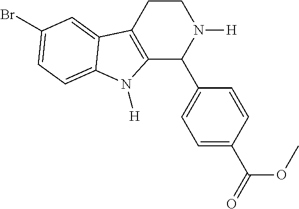 [SMILES: COC(=O)c1ccc(C2NCCc3c2[nH]c2ccc(Br)cc32)cc1] | 586 |
| 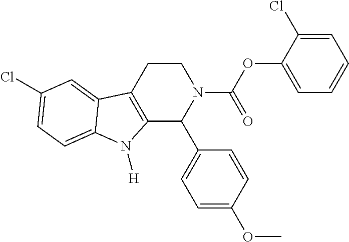 [SMILES: COc1ccc(C2c3[nH]c4ccc(Cl)cc4c3CCN2C(=O)Oc2ccccc2Cl)cc1] | 587 |
| 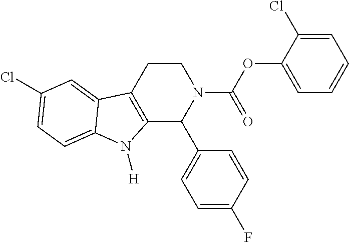 [SMILES: O=C(Oc1ccccc1Cl)N1CCc2c([nH]c3ccc(Cl)cc23)C1c1ccc(F)cc1] | 588 |
| 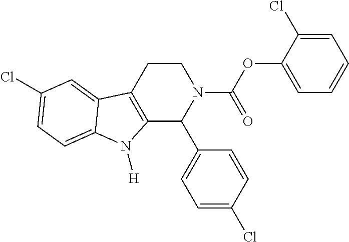 [SMILES: O=C(Oc1ccccc1Cl)N1CCc2c([nH]c3ccc(Cl)cc23)C1c1ccc(Cl)cc1] | 589 |
| 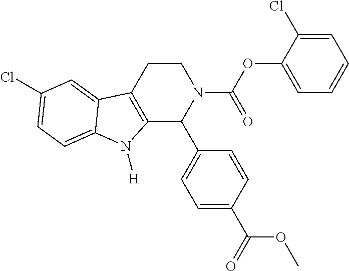 [SMILES: COC(=O)c1ccc(C2c3[nH]c4ccc(Cl)cc4c3CCN2C(=O)Oc2ccccc2Cl)cc1] | 590 |
| 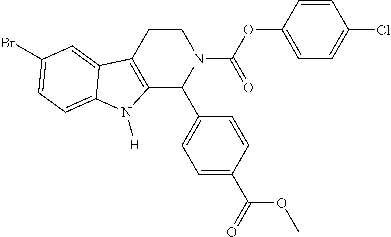 [SMILES: COC(=O)c1ccc(C2c3[nH]c4ccc(Br)cc4c3CCN2C(=O)Oc2ccc(Cl)cc2)cc1] | 591 |
| 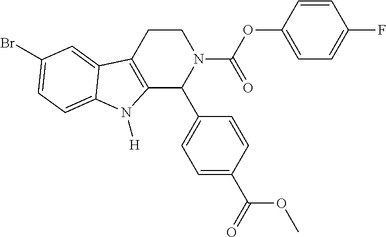 [SMILES: COC(=O)c1ccc(C2c3[nH]c4ccc(Br)cc4c3CCN2C(=O)Oc2ccc(F)cc2)cc1] | 592 |
| 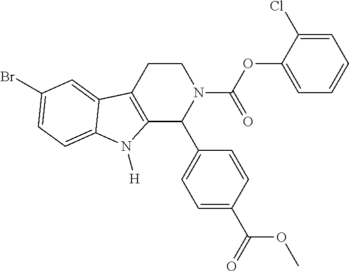 [SMILES: COC(=O)c1ccc(C2c3[nH]c4ccc(Br)cc4c3CCN2C(=O)Oc2ccccc2Cl)cc1] | 593 |
| 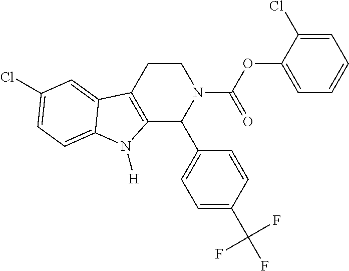 [SMILES: O=C(Oc1ccccc1Cl)N1CCc2c([nH]c3ccc(Cl)cc23)C1c1ccc(C(F)(F)F)cc1] | 594 |
| 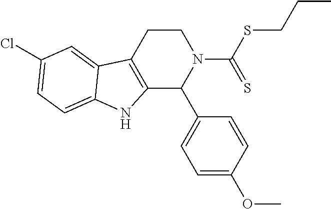 [SMILES: CCCSC(=S)N1CCc2c([nH]c3ccc(Cl)cc23)C1c1ccc(OC)cc1] | 595 |
| 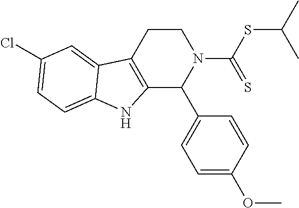 [SMILES: COc1ccc(C2c3[nH]c4ccc(Cl)cc4c3CCN2C(=S)SC(C)C)cc1] | 596 |
| 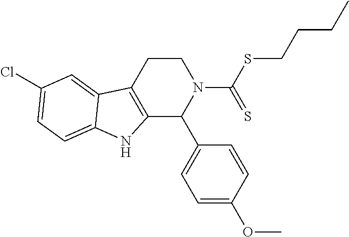 [SMILES: CCCCSC(=S)N1CCc2c([nH]c3ccc(Cl)cc23)C1c1ccc(OC)cc1] | 597 |
| 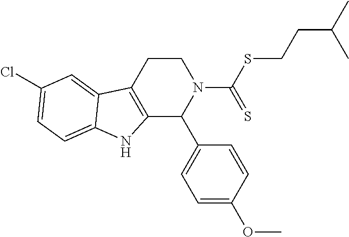 [SMILES: COc1ccc(C2c3[nH]c4ccc(Cl)cc4c3CCN2C(=S)SCCC(C)C)cc1] | 598 |
| 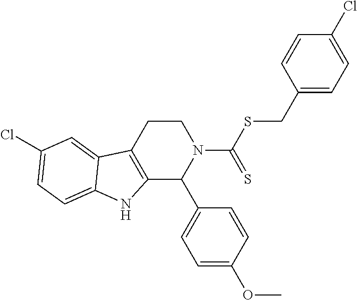 [SMILES: COc1ccc(C2c3[nH]c4ccc(Cl)cc4c3CCN2C(=S)SCc2ccc(Cl)cc2)cc1] | 599 |
| 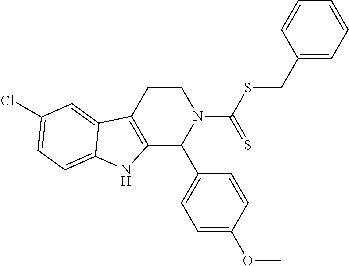 [SMILES: COc1ccc(C2c3[nH]c4ccc(Cl)cc4c3CCN2C(=S)SCc2ccccc2)cc1] | 600 |
| 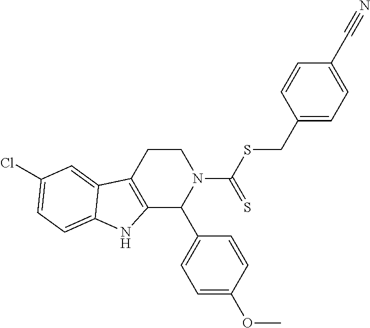 [SMILES: COc1ccc(C2c3[nH]c4ccc(Cl)cc4c3CCN2C(=S)SCc2ccc(C#N)cc2)cc1] | 601 |
| 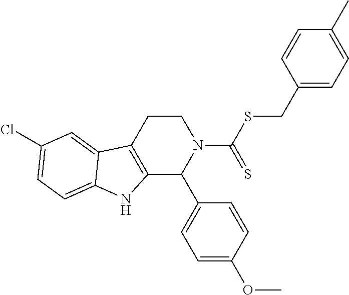 [SMILES: COc1ccc(C2c3[nH]c4ccc(Cl)cc4c3CCN2C(=S)SCc2ccc(C)cc2)cc1] | 602 |
| 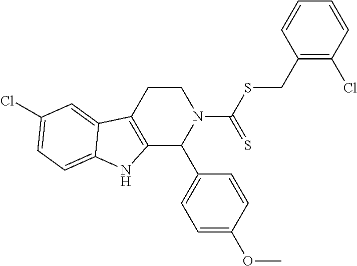 [SMILES: COc1ccc(C2c3[nH]c4ccc(Cl)cc4c3CCN2C(=S)SCc2ccccc2Cl)cc1] | 603 |
| 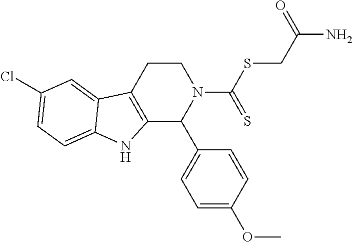 [SMILES: COc1ccc(C2c3[nH]c4ccc(Cl)cc4c3CCN2C(=S)SCC(N)=O)cc1] | 604 |
| 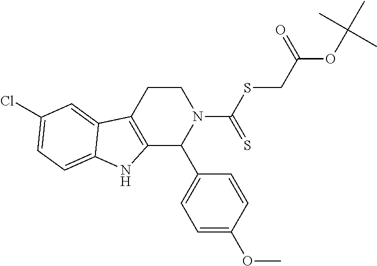 [SMILES: COc1ccc(C2c3[nH]c4ccc(Cl)cc4c3CCN2C(=S)SCC(=O)OC(C)(C)C)cc1] | 605 |
| 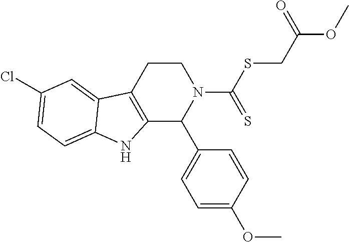 [SMILES: COC(=O)CSC(=S)N1CCc2c([nH]c3ccc(Cl)cc23)C1c1ccc(OC)cc1] | 606 |
| 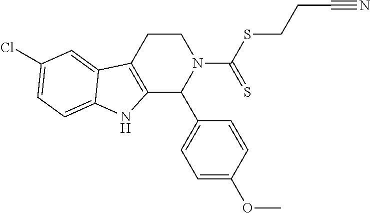 [SMILES: COc1ccc(C2c3[nH]c4ccc(Cl)cc4c3CCN2C(=S)SCCC#N)cc1] | 607 |
| 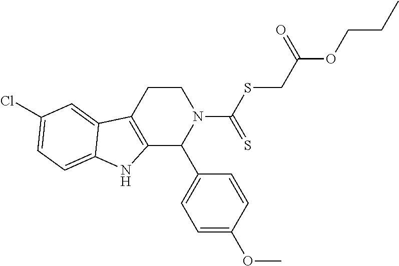 [SMILES: CCCOC(=O)CSC(=S)N1CCc2c([nH]c3ccc(Cl)cc23)C1c1ccc(OC)cc1] | 608 |
| 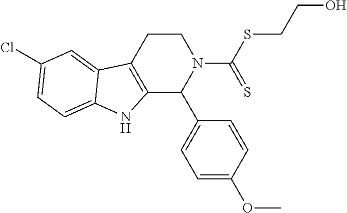 [SMILES: COc1ccc(C2c3[nH]c4ccc(Cl)cc4c3CCN2C(=S)SCCO)cc1] | 609 |
| 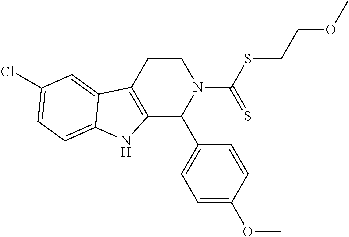 [SMILES: COCCSC(=S)N1CCc2c([nH]c3ccc(Cl)cc23)C1c1ccc(OC)cc1] | 610 |
| 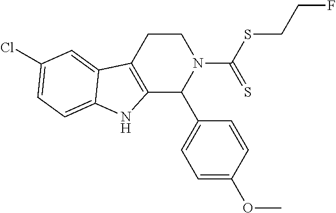 [SMILES: COc1ccc(C2c3[nH]c4ccc(Cl)cc4c3CCN2C(=S)SCCF)cc1] | 611 |
| 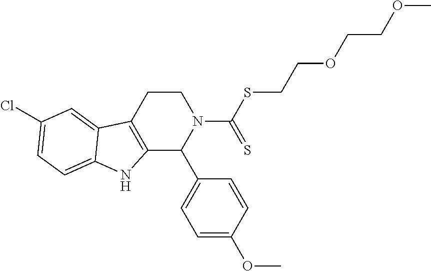 [SMILES: COCCOCCSC(=S)N1CCc2c([nH]c3ccc(Cl)cc23)C1c1ccc(OC)cc1] | 612 |
| 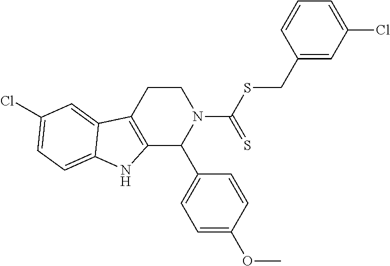 [SMILES: COc1ccc(C2c3[nH]c4ccc(Cl)cc4c3CCN2C(=S)SCc2cccc(Cl)c2)cc1] | 613 |
| 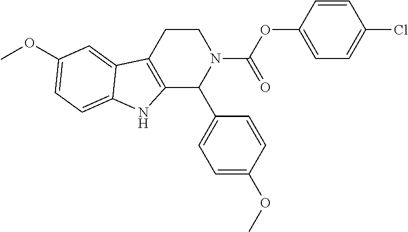 [SMILES: COc1ccc(C2c3[nH]c4ccc(OC)cc4c3CCN2C(=O)Oc2ccc(Cl)cc2)cc1] | 614 |
| 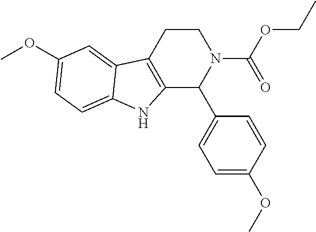 [SMILES: CCOC(=O)N1CCc2c([nH]c3ccc(OC)cc23)C1c1ccc(OC)cc1] | 615 |
| 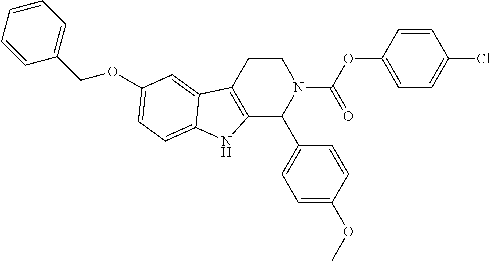 [SMILES: COc1ccc(C2c3[nH]c4ccc(OCc5ccccc5)cc4c3CCN2C(=O)Oc2ccc(Cl)cc2)cc1] | 616 |
| 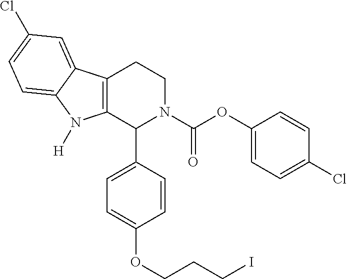 [SMILES: O=C(Oc1ccc(Cl)cc1)N1CCc2c([nH]c3ccc(Cl)cc23)C1c1ccc(OCCCI)cc1] | 617 |
| 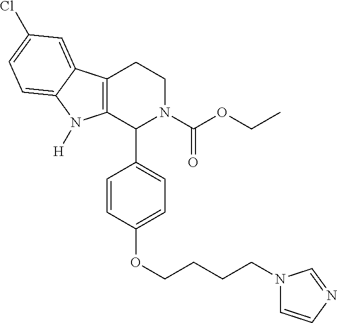 [SMILES: CCOC(=O)N1CCc2c([nH]c3ccc(Cl)cc23)C1c1ccc(OCCCCn2ccnc2)cc1] | 618 |
| 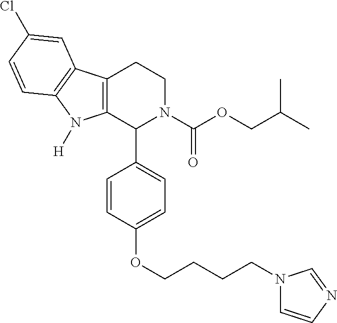 [SMILES: CC(C)COC(=O)N1CCc2c([nH]c3ccc(Cl)cc23)C1c1ccc(OCCCCn2ccnc2)cc1] | 619 |
| 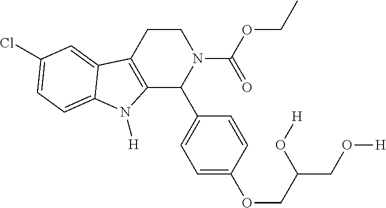 [SMILES: CCOC(=O)N1CCc2c([nH]c3ccc(Cl)cc23)C1c1ccc(OCC(O)CO)cc1] | 620 |
| 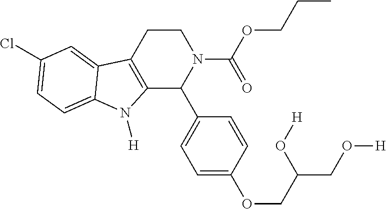 [SMILES: CCCOC(=O)N1CCc2c([nH]c3ccc(Cl)cc23)C1c1ccc(OCC(O)CO)cc1] | 621 |
| 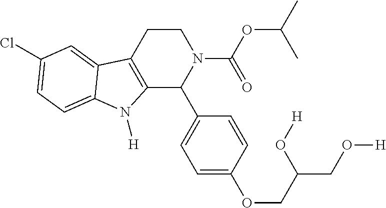 [SMILES: CC(C)OC(=O)N1CCc2c([nH]c3ccc(Cl)cc23)C1c1ccc(OCC(O)CO)cc1] | 622 |
| 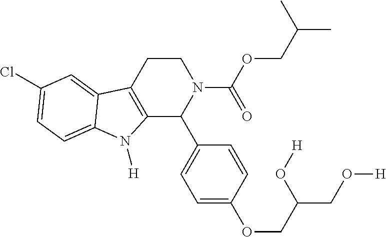 [SMILES: CC(C)COC(=O)N1CCc2c([nH]c3ccc(Cl)cc23)C1c1ccc(OCC(O)CO)cc1] | 623 |
| 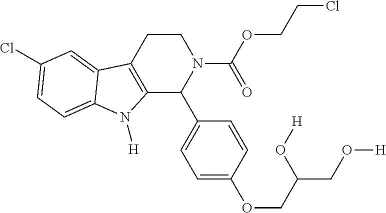 [SMILES: O=C(OCCCl)N1CCc2c([nH]c3ccc(Cl)cc23)C1c1ccc(OCC(O)CO)cc1] | 624 |
| 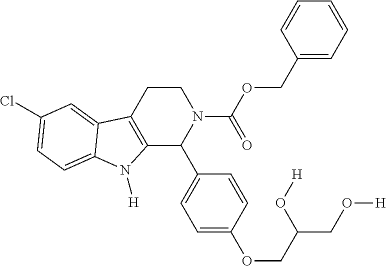 [SMILES: O=C(OCc1ccccc1)N1CCc2c([nH]c3ccc(Cl)cc23)C1c1ccc(OCC(O)CO)cc1] | 625 |
| 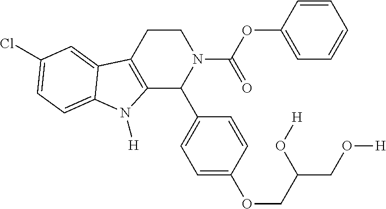 [SMILES: O=C(Oc1ccccc1)N1CCc2c([nH]c3ccc(Cl)cc23)C1c1ccc(OCC(O)CO)cc1] | 626 |
| 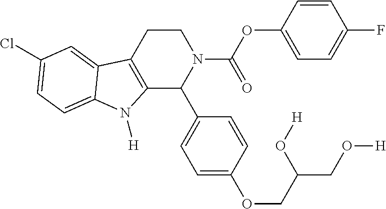 [SMILES: O=C(Oc1ccc(F)cc1)N1CCc2c([nH]c3ccc(Cl)cc23)C1c1ccc(OCC(O)CO)cc1] | 627 |
| 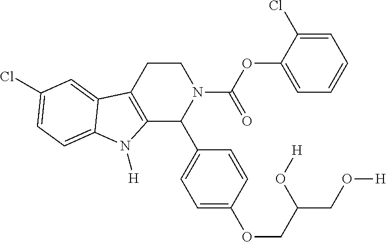 [SMILES: O=C(Oc1ccccc1Cl)N1CCc2c([nH]c3ccc(Cl)cc23)C1c1ccc(OCC(O)CO)cc1] | 628 |
| 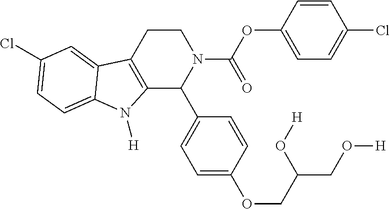 [SMILES: O=C(Oc1ccc(Cl)cc1)N1CCc2c([nH]c3ccc(Cl)cc23)C1c1ccc(OCC(O)CO)cc1] | 629 |
| 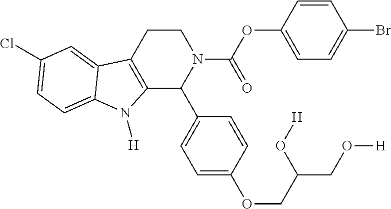 [SMILES: O=C(Oc1ccc(Br)cc1)N1CCc2c([nH]c3ccc(Cl)cc23)C1c1ccc(OCC(O)CO)cc1] | 630 |
| 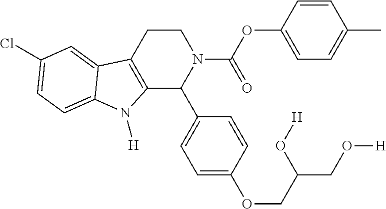 [SMILES: Cc1ccc(OC(=O)N2CCc3c([nH]c4ccc(Cl)cc34)C2c2ccc(OCC(O)CO)cc2)cc1] | 631 |
| 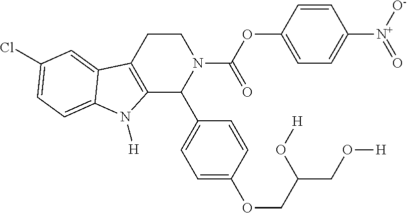 [SMILES: O=C(Oc1ccc([N+](=O)[O-])cc1)N1CCc2c([nH]c3ccc(Cl)cc23)C1c1ccc(OCC(O)CO)cc1] | 632 |
| 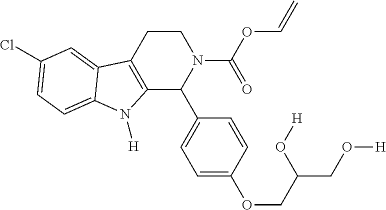 [SMILES: C=COC(=O)N1CCc2c([nH]c3ccc(Cl)cc23)C1c1ccc(OCC(O)CO)cc1] | 633 |
| 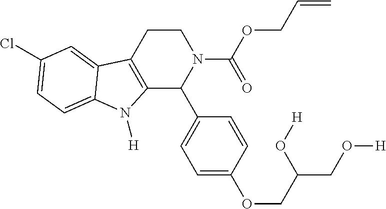 [SMILES: C=CCOC(=O)N1CCc2c([nH]c3ccc(Cl)cc23)C1c1ccc(OCC(O)CO)cc1] | 634 |
| 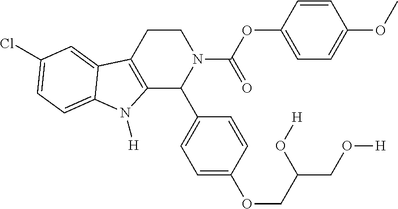 [SMILES: COc1ccc(OC(=O)N2CCc3c([nH]c4ccc(Cl)cc34)C2c2ccc(OCC(O)CO)cc2)cc1] | 635 |
| 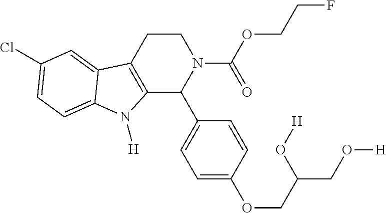 [SMILES: O=C(OCCF)N1CCc2c([nH]c3ccc(Cl)cc23)C1c1ccc(OCC(O)CO)cc1] | 636 |
| 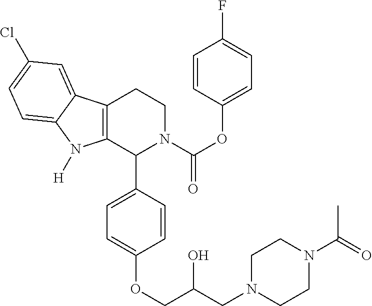 [SMILES: CC(=O)N1CCN(CC(O)COc2ccc(C3c4[nH]c5ccc(Cl)cc5c4CCN3C(=O)Oc3ccc(F)cc3)cc2)CC1] | 637 |
| 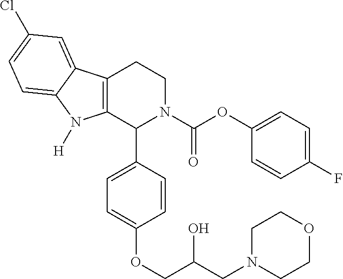 [SMILES: O=C(Oc1ccc(F)cc1)N1CCc2c([nH]c3ccc(Cl)cc23)C1c1ccc(OCC(O)CN2CCOCC2)cc1] | 638 |
| 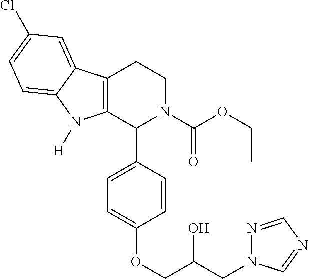 [SMILES: CCOC(=O)N1CCc2c([nH]c3ccc(Cl)cc23)C1c1ccc(OCC(O)Cn2cncn2)cc1] | 639 |
| 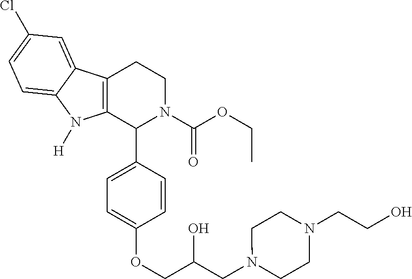 [SMILES: CCOC(=O)N1CCc2c([nH]c3ccc(Cl)cc23)C1c1ccc(OCC(O)CN2CCN(CCO)CC2)cc1] | 640 |
| 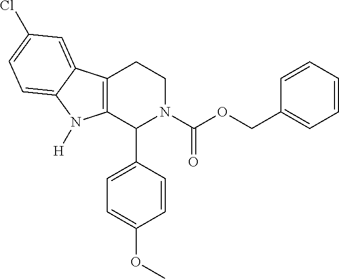 [SMILES: COc1ccc(C2c3[nH]c4ccc(Cl)cc4c3CCN2C(=O)OCc2ccccc2)cc1] | 641 |
| 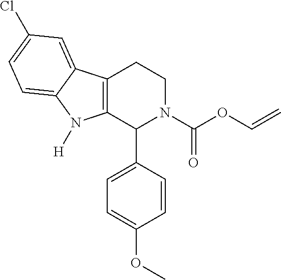 [SMILES: C=COC(=O)N1CCc2c([nH]c3ccc(Cl)cc23)C1c1ccc(OC)cc1] | 642 |
| 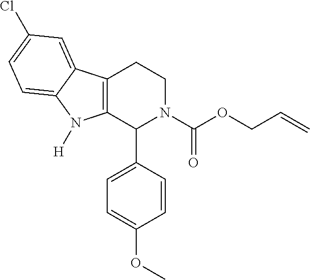 [SMILES: C=CCOC(=O)N1CCc2c([nH]c3ccc(Cl)cc23)C1c1ccc(OC)cc1] | 643 |
| 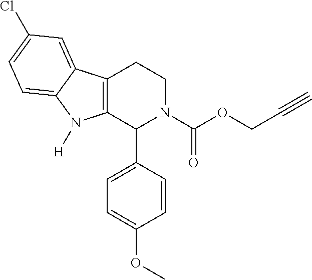 [SMILES: C#CCOC(=O)N1CCc2c([nH]c3ccc(Cl)cc23)C1c1ccc(OC)cc1] | 644 |
| 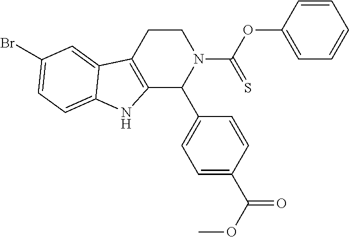 [SMILES: COC(=O)c1ccc(C2c3[nH]c4ccc(Br)cc4c3CCN2C(=S)Oc2ccccc2)cc1] | 645 |
| 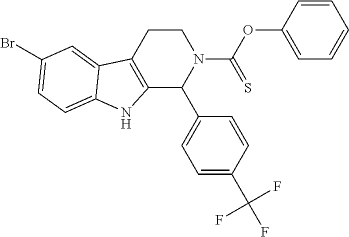 [SMILES: FC(F)(F)c1ccc(C2c3[nH]c4ccc(Br)cc4c3CCN2C(=S)Oc2ccccc2)cc1] | 646 |
| 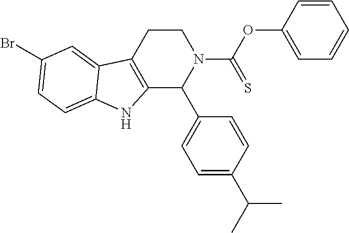 [SMILES: CC(C)c1ccc(C2c3[nH]c4ccc(Br)cc4c3CCN2C(=S)Oc2ccccc2)cc1] | 647 |
| 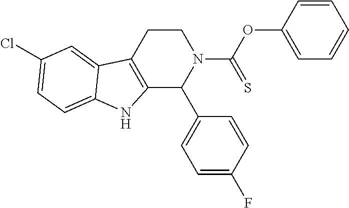 [SMILES: Fc1ccc(C2c3[nH]c4ccc(Cl)cc4c3CCN2C(=S)Oc2ccccc2)cc1] | 648 |
| 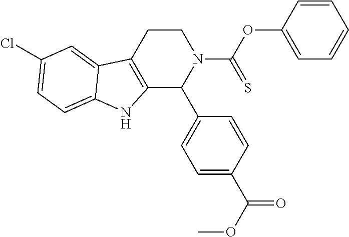 [SMILES: COC(=O)c1ccc(C2c3[nH]c4ccc(Cl)cc4c3CCN2C(=S)Oc2ccccc2)cc1] | 649 |
| 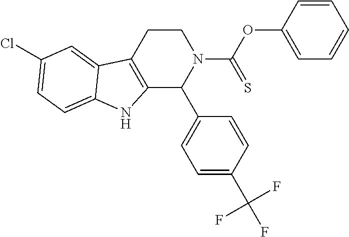 [SMILES: FC(F)(F)c1ccc(C2c3[nH]c4ccc(Cl)cc4c3CCN2C(=S)Oc2ccccc2)cc1] | 650 |
| 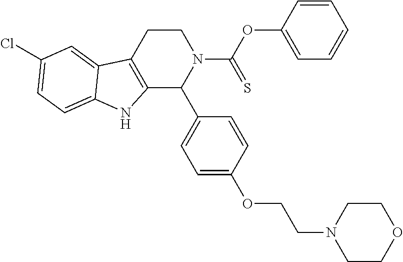 [SMILES: S=C(Oc1ccccc1)N1CCc2c([nH]c3ccc(Cl)cc23)C1c1ccc(OCCN2CCOCC2)cc1] | 651 |
| 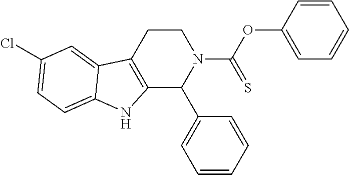 [SMILES: S=C(Oc1ccccc1)N1CCc2c([nH]c3ccc(Cl)cc23)C1c1ccccc1] | 652 |
| 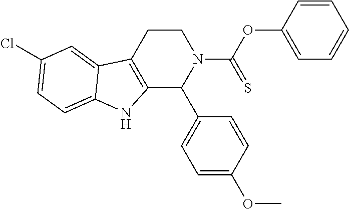 [SMILES: COc1ccc(C2c3[nH]c4ccc(Cl)cc4c3CCN2C(=S)Oc2ccccc2)cc1] | 653 |
| 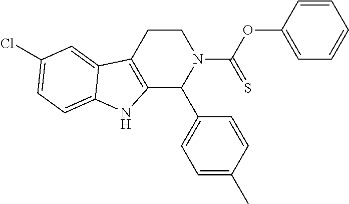 [SMILES: Cc1ccc(C2c3[nH]c4ccc(Cl)cc4c3CCN2C(=S)Oc2ccccc2)cc1] | 654 |
| 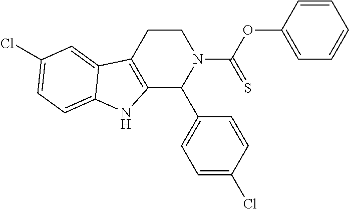 [SMILES: S=C(Oc1ccccc1)N1CCc2c([nH]c3ccc(Cl)cc23)C1c1ccc(Cl)cc1] | 655 |
| 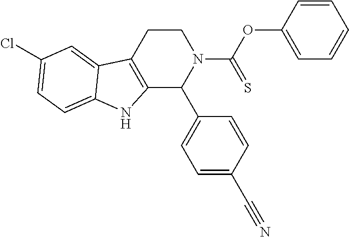 [SMILES: N#Cc1ccc(C2c3[nH]c4ccc(Cl)cc4c3CCN2C(=S)Oc2ccccc2)cc1] | 656 |
| 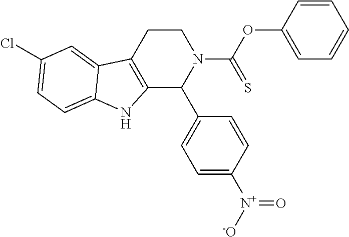 | 657 |
| 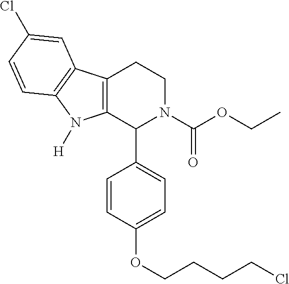 [SMILES: CCOC(=O)N1CCc2c([nH]c3ccc(Cl)cc23)C1c1ccc(OCCCCCl)cc1] | 658 |
| 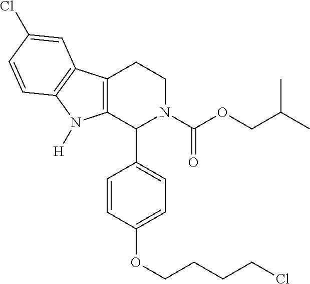 [SMILES: CC(C)COC(=O)N1CCc2c([nH]c3ccc(Cl)cc23)C1c1ccc(OCCCCCl)cc1] | 659 |
| 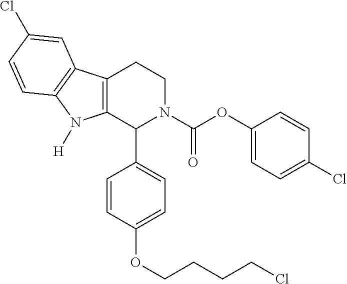 [SMILES: O=C(Oc1ccc(Cl)cc1)N1CCc2c([nH]c3ccc(Cl)cc23)C1c1ccc(OCCCCCl)cc1] | 660 |
| 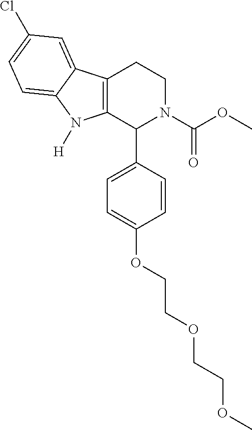 [SMILES: COCCOCCOc1ccc(C2c3[nH]c4ccc(Cl)cc4c3CCN2C(=O)OC)cc1] | 661 |
| 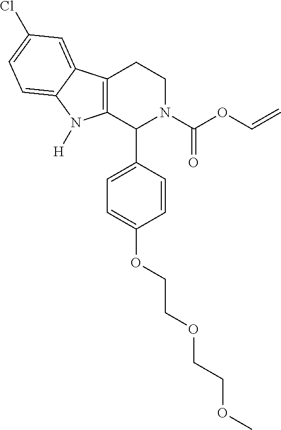 [SMILES: C=COC(=O)N1CCc2c([nH]c3ccc(Cl)cc23)C1c1ccc(OCCOCCOC)cc1] | 662 |
| 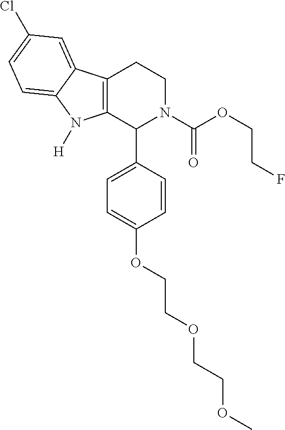 [SMILES: COCCOCCOc1ccc(C2c3[nH]c4ccc(Cl)cc4c3CCN2C(=O)OCCF)cc1] | 663 |
| 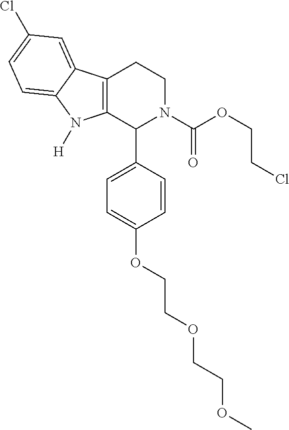 [SMILES: COCCOCCOc1ccc(C2c3[nH]c4ccc(Cl)cc4c3CCN2C(=O)OCCCl)cc1] | 664 |
| 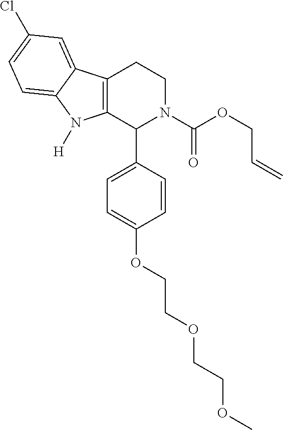 [SMILES: C=CCOC(=O)N1CCc2c([nH]c3ccc(Cl)cc23)C1c1ccc(OCCOCCOC)cc1] | 665 |
| 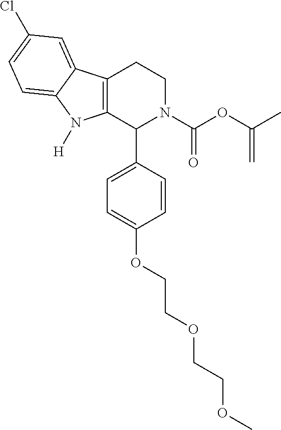 [SMILES: C=C(C)OC(=O)N1CCc2c([nH]c3ccc(Cl)cc23)C1c1ccc(OCCOCCOC)cc1] | 666 |
| 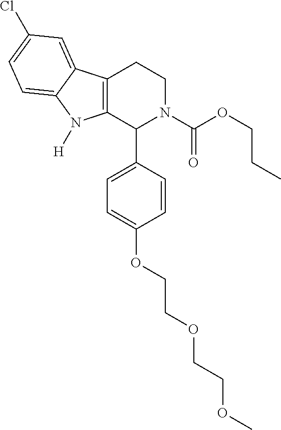 [SMILES: CCCOC(=O)N1CCc2c([nH]c3ccc(Cl)cc23)C1c1ccc(OCCOCCOC)cc1] | 667 |
| 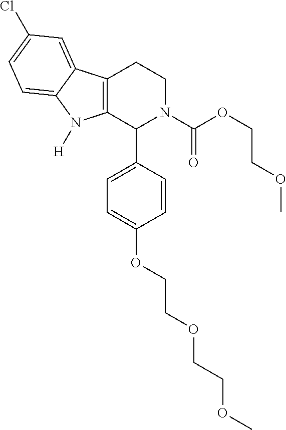 [SMILES: COCCOCCOc1ccc(C2c3[nH]c4ccc(Cl)cc4c3CCN2C(=O)OCCOC)cc1] | 668 |
| 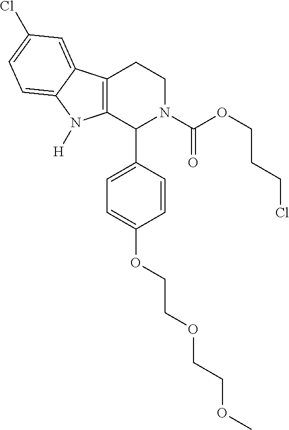 [SMILES: COCCOCCOc1ccc(C2c3[nH]c4ccc(Cl)cc4c3CCN2C(=O)OCCCCl)cc1] | 669 |
| 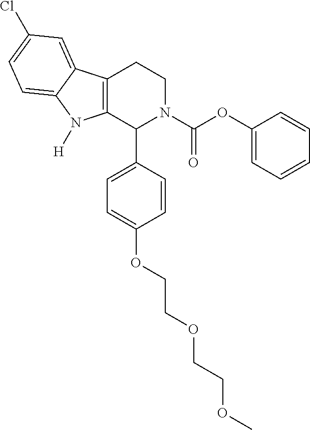 [SMILES: COCCOCCOc1ccc(C2c3[nH]c4ccc(Cl)cc4c3CCN2C(=O)Oc2ccccc2)cc1] | 670 |
| 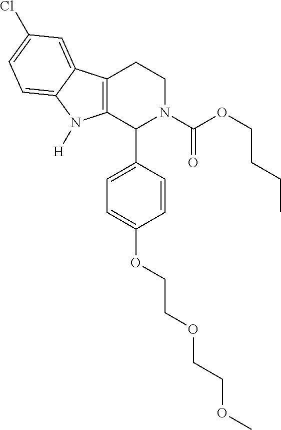 [SMILES: CCCCOC(=O)N1CCc2c([nH]c3ccc(Cl)cc23)C1c1ccc(OCCOCCOC)cc1] | 671 |
| 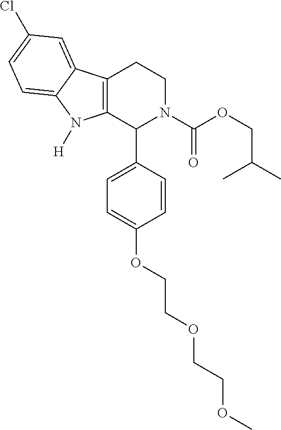 [SMILES: COCCOCCOc1ccc(C2c3[nH]c4ccc(Cl)cc4c3CCN2C(=O)OCC(C)C)cc1] | 672 |
| 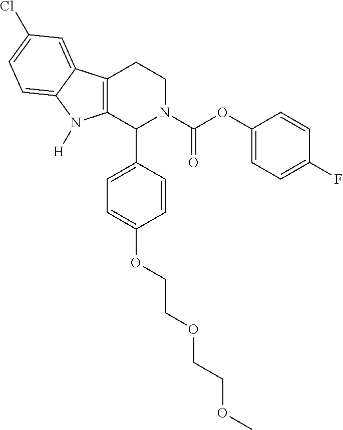 [SMILES: COCCOCCOc1ccc(C2c3[nH]c4ccc(Cl)cc4c3CCN2C(=O)Oc2ccc(F)cc2)cc1] | 673 |
| 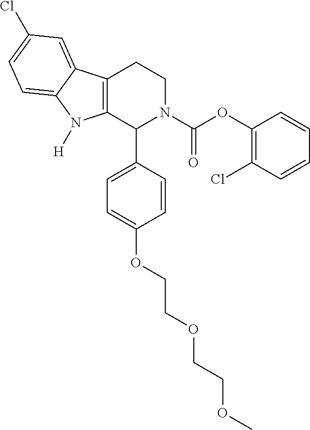 [SMILES: COCCOCCOc1ccc(C2c3[nH]c4ccc(Cl)cc4c3CCN2C(=O)Oc2ccccc2Cl)cc1] | 674 |
| 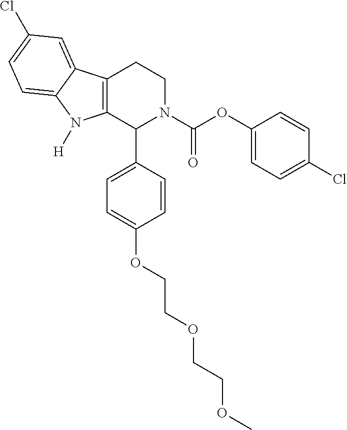 [SMILES: COCCOCCOc1ccc(C2c3[nH]c4ccc(Cl)cc4c3CCN2C(=O)Oc2ccc(Cl)cc2)cc1] | 675 |
| 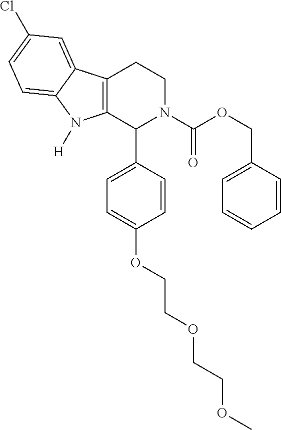 [SMILES: COCCOCCOc1ccc(C2c3[nH]c4ccc(Cl)cc4c3CCN2C(=O)OCc2ccccc2)cc1] | 676 |
| 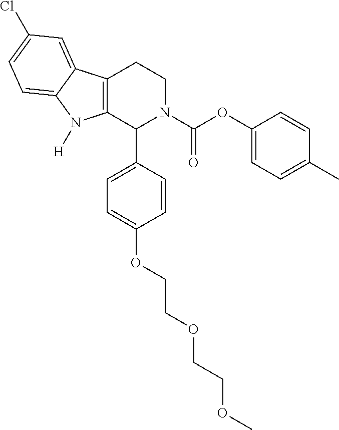 [SMILES: COCCOCCOc1ccc(C2c3[nH]c4ccc(Cl)cc4c3CCN2C(=O)Oc2ccc(C)cc2)cc1] | 677 |
| 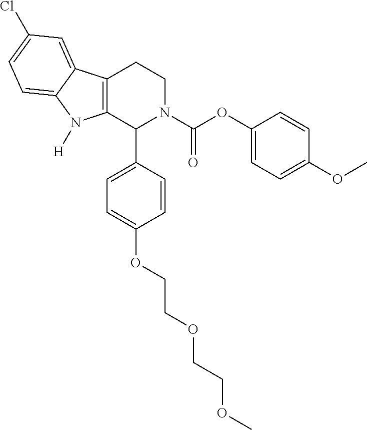 [SMILES: COCCOCCOc1ccc(C2c3[nH]c4ccc(Cl)cc4c3CCN2C(=O)Oc2ccc(OC)cc2)cc1] | 678 |
| 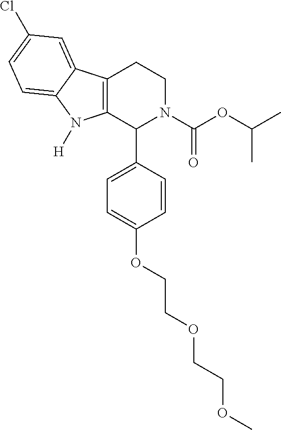 [SMILES: COCCOCCOc1ccc(C2c3[nH]c4ccc(Cl)cc4c3CCN2C(=O)OC(C)C)cc1] | 679 |
| 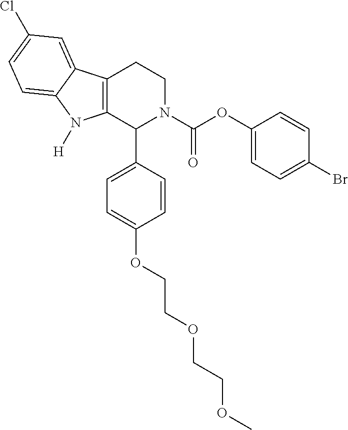 [SMILES: COCCOCCOc1ccc(C2c3[nH]c4ccc(Cl)cc4c3CCN2C(=O)Oc2ccc(Br)cc2)cc1] | 680 |
| 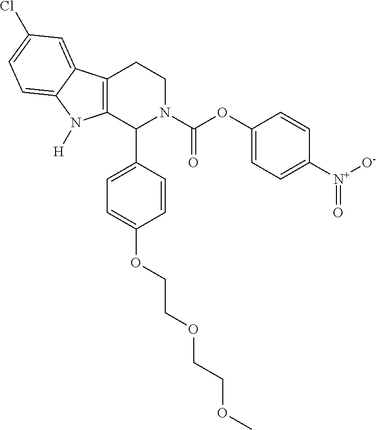 [SMILES: COCCOCCOc1ccc(C2c3[nH]c4ccc(Cl)cc4c3CCN2C(=O)Oc2ccc([N+](=O)[O-])cc2)cc1] | 681 |
| 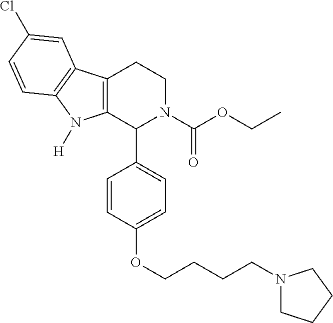 [SMILES: CCOC(=O)N1CCc2c([nH]c3ccc(Cl)cc23)C1c1ccc(OCCCCN2CCCC2)cc1] | 682 |
| 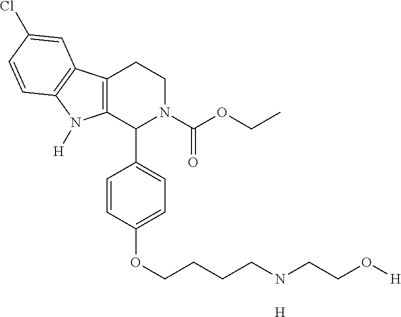 | 683 |
| 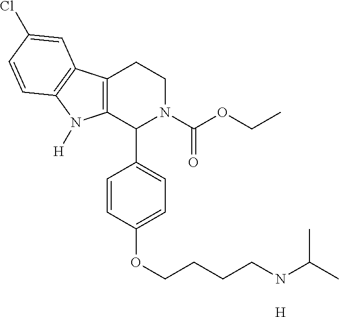 | 684 |
| 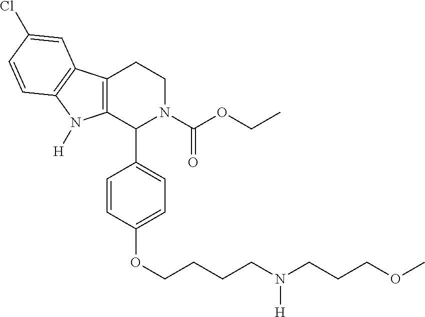 [SMILES: CCOC(=O)N1CCc2c([nH]c3ccc(Cl)cc23)C1c1ccc(OCCCCNCCCOC)cc1] | 685 |
| 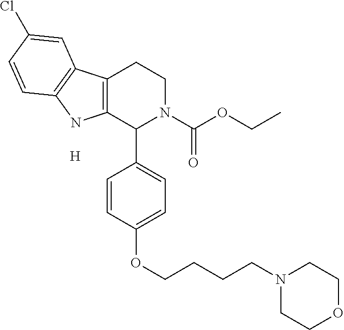 | 686 |
| 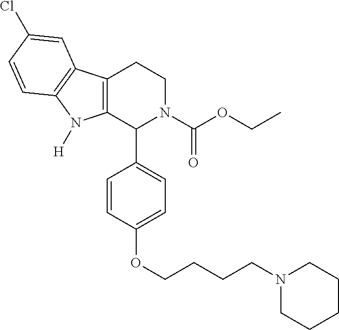 [SMILES: CCOC(=O)N1CCc2c([nH]c3ccc(Cl)cc23)C1c1ccc(OCCCCN2CCCCC2)cc1] | 687 |
| 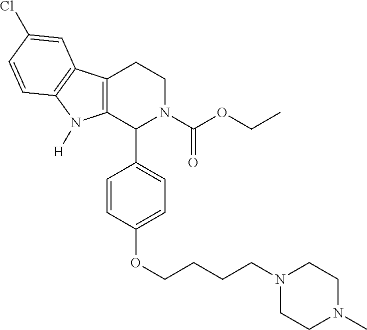 [SMILES: CCOC(=O)N1CCc2c([nH]c3ccc(Cl)cc23)C1c1ccc(OCCCCN2CCN(C)CC2)cc1] | 688 |
| 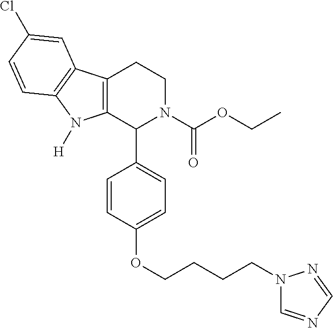 [SMILES: CCOC(=O)N1CCc2c([nH]c3ccc(Cl)cc23)C1c1ccc(OCCCCn2cncn2)cc1] | 689 |
| 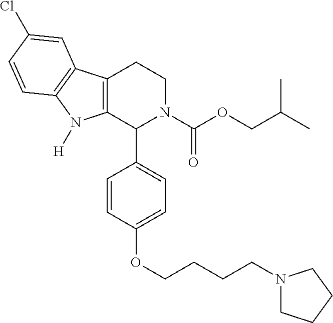 [SMILES: CC(C)COC(=O)N1CCc2c([nH]c3ccc(Cl)cc23)C1c1ccc(OCCCCN2CCCC2)cc1] | 690 |
| 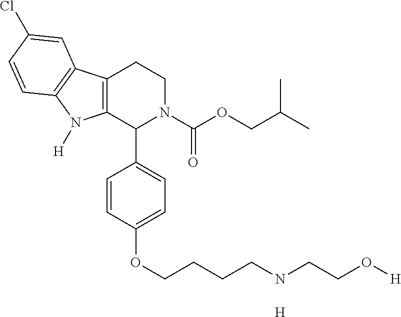 | 691 |
| 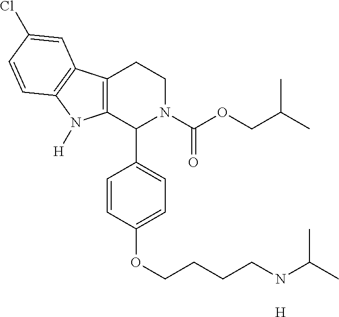 | 692 |
| 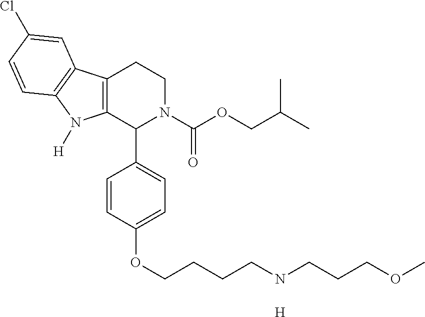 | 693 |
| 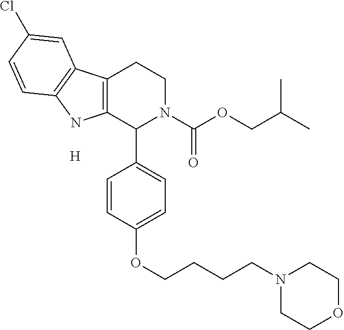 | 694 |
| 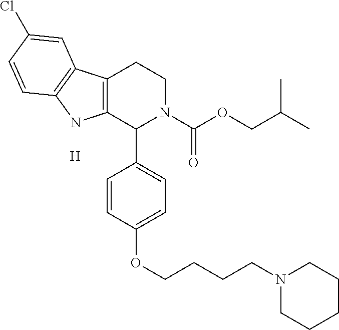 | 695 |
| 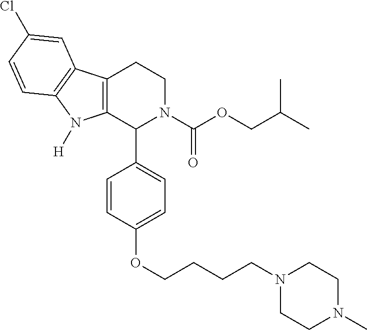 [SMILES: CC(C)COC(=O)N1CCc2c([nH]c3ccc(Cl)cc23)C1c1ccc(OCCCCN2CCN(C)CC2)cc1] | 696 |
| 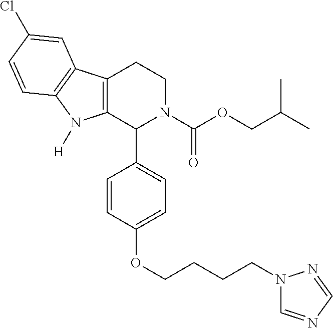 [SMILES: CC(C)COC(=O)N1CCc2c([nH]c3ccc(Cl)cc23)C1c1ccc(OCCCCn2cncn2)cc1] | 697 |
| 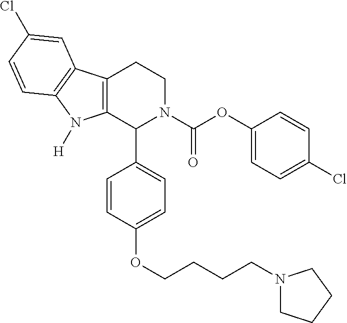 [SMILES: O=C(Oc1ccc(Cl)cc1)N1CCc2c([nH]c3ccc(Cl)cc23)C1c1ccc(OCCCCN2CCCC2)cc1] | 698 |
| 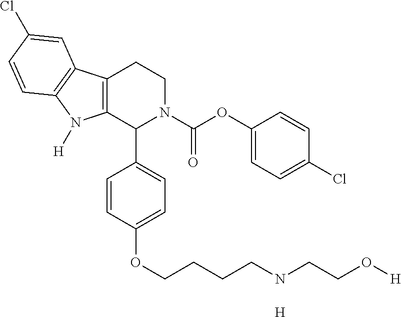 | 699 |
| 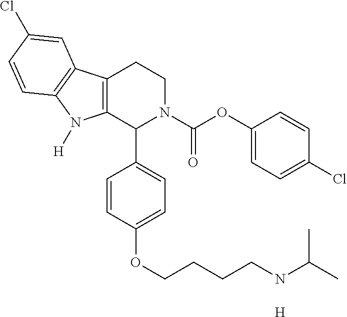 | 700 |
| 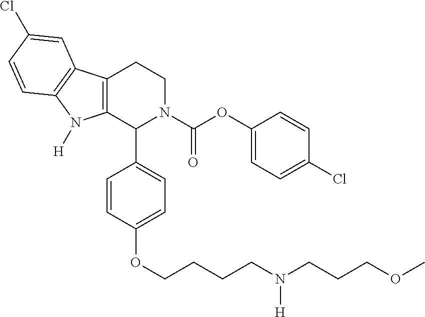 [SMILES: COCCCNCCCCOc1ccc(C2c3[nH]c4ccc(Cl)cc4c3CCN2C(=O)Oc2ccc(Cl)cc2)cc1] | 701 |
| 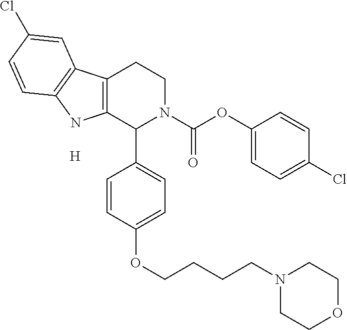 | 702 |
| 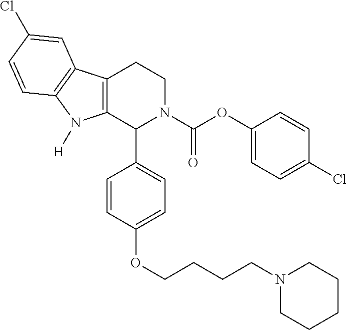 [SMILES: O=C(Oc1ccc(Cl)cc1)N1CCc2c([nH]c3ccc(Cl)cc23)C1c1ccc(OCCCCN2CCCCC2)cc1] | 703 |
| 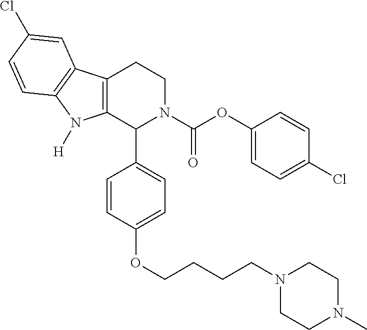 [SMILES: CN1CCN(CCCCOc2ccc(C3c4[nH]c5ccc(Cl)cc5c4CCN3C(=O)Oc3ccc(Cl)cc3)cc2)CC1] | 704 |
| 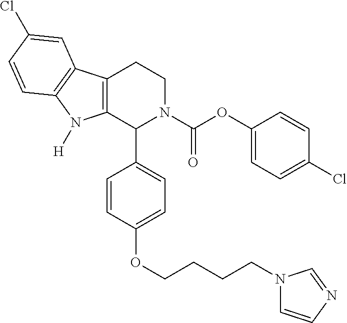 [SMILES: O=C(Oc1ccc(Cl)cc1)N1CCc2c([nH]c3ccc(Cl)cc23)C1c1ccc(OCCCCn2ccnc2)cc1] | 705 |
| 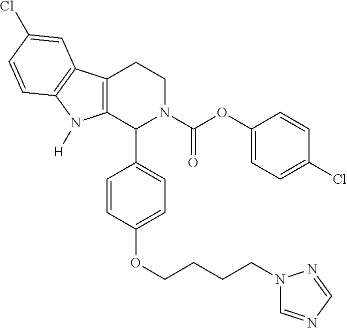 [SMILES: O=C(Oc1ccc(Cl)cc1)N1CCc2c([nH]c3ccc(Cl)cc23)C1c1ccc(OCCCCn2cncn2)cc1] | 706 |
| 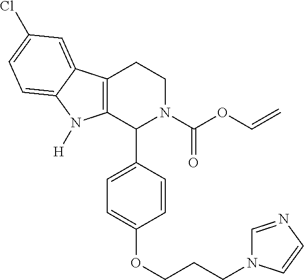 [SMILES: C=COC(=O)N1CCc2c([nH]c3ccc(Cl)cc23)C1c1ccc(OCCCn2ccnc2)cc1] | 707 |
| 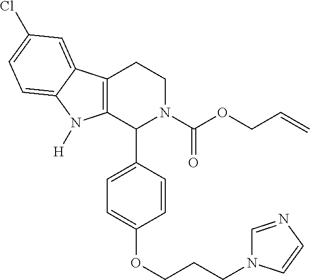 [SMILES: C=CCOC(=O)N1CCc2c([nH]c3ccc(Cl)cc23)C1c1ccc(OCCCn2ccnc2)cc1] | 708 |
| 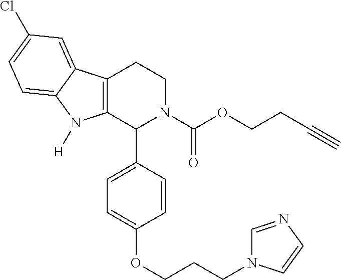 [SMILES: C#CCCOC(=O)N1CCc2c([nH]c3ccc(Cl)cc23)C1c1ccc(OCCCn2ccnc2)cc1] | 709 |
| 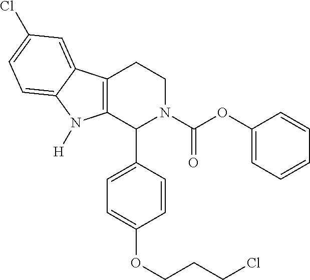 [SMILES: O=C(Oc1ccccc1)N1CCc2c([nH]c3ccc(Cl)cc23)C1c1ccc(OCCCCl)cc1] | 710 |
| 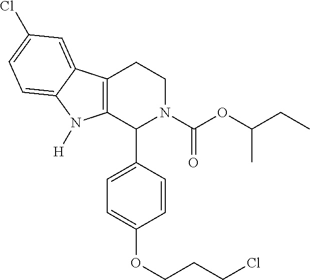 [SMILES: CCC(C)OC(=O)N1CCc2c([nH]c3ccc(Cl)cc23)C1c1ccc(OCCCCl)cc1] | 711 |
| 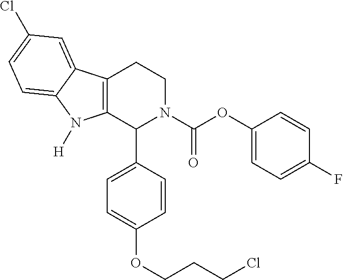 [SMILES: O=C(Oc1ccc(F)cc1)N1CCc2c([nH]c3ccc(Cl)cc23)C1c1ccc(OCCCCl)cc1] | 712 |
| 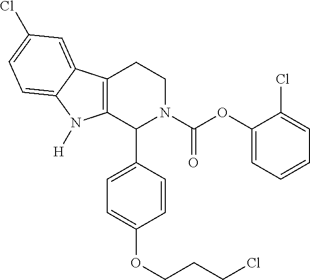 [SMILES: O=C(Oc1ccccc1Cl)N1CCc2c([nH]c3ccc(Cl)cc23)C1c1ccc(OCCCCl)cc1] | 713 |
| 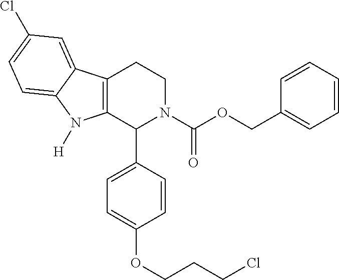 [SMILES: O=C(OCc1ccccc1)N1CCc2c([nH]c3ccc(Cl)cc23)C1c1ccc(OCCCCl)cc1] | 714 |
| 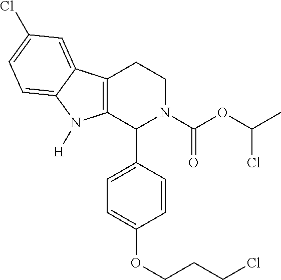 [SMILES: CC(Cl)OC(=O)N1CCc2c([nH]c3ccc(Cl)cc23)C1c1ccc(OCCCCl)cc1] | 715 |
| 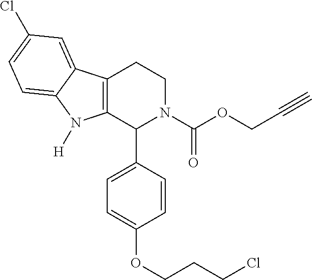 [SMILES: C#CCOC(=O)N1CCc2c([nH]c3ccc(Cl)cc23)C1c1ccc(OCCCCl)cc1] | 716 |
| 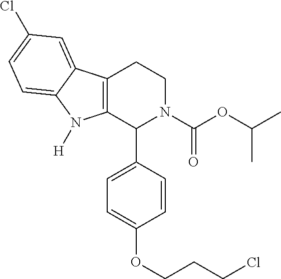 [SMILES: CC(C)OC(=O)N1CCc2c([nH]c3ccc(Cl)cc23)C1c1ccc(OCCCCl)cc1] | 717 |
| 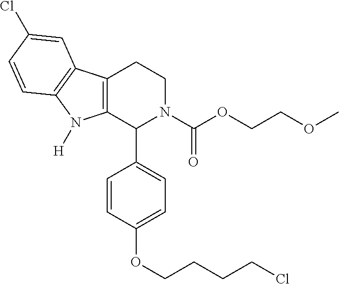 [SMILES: COCCOC(=O)N1CCc2c([nH]c3ccc(Cl)cc23)C1c1ccc(OCCCCCl)cc1] | 718 |
| 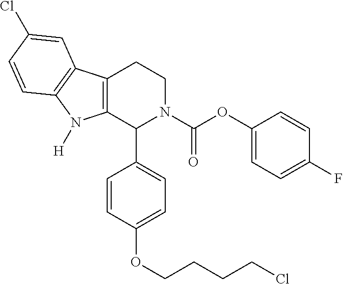 [SMILES: O=C(Oc1ccc(F)cc1)N1CCc2c([nH]c3ccc(Cl)cc23)C1c1ccc(OCCCCCl)cc1] | 719 |
| 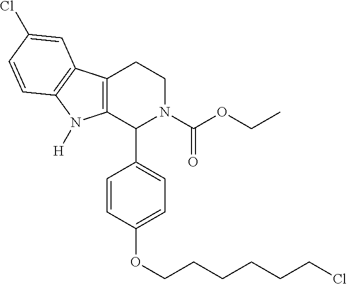 [SMILES: CCOC(=O)N1CCc2c([nH]c3ccc(Cl)cc23)C1c1ccc(OCCCCCCCl)cc1] | 720 |
| 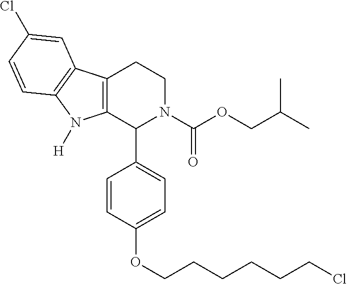 [SMILES: CC(C)COC(=O)N1CCc2c([nH]c3ccc(Cl)cc23)C1c1ccc(OCCCCCCCl)cc1] | 721 |
| 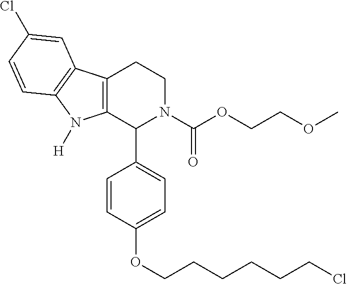 [SMILES: COCCOC(=O)N1CCc2c([nH]c3ccc(Cl)cc23)C1c1ccc(OCCCCCCCl)cc1] | 722 |
| 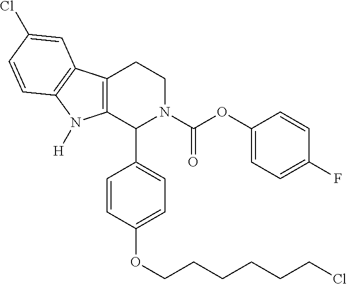 [SMILES: O=C(Oc1ccc(F)cc1)N1CCc2c([nH]c3ccc(Cl)cc23)C1c1ccc(OCCCCCCCl)cc1] | 723 |
| 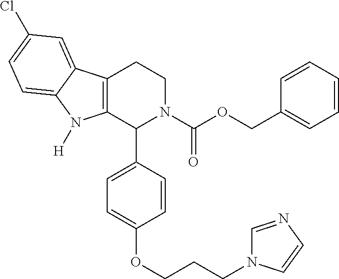 [SMILES: O=C(OCc1ccccc1)N1CCc2c([nH]c3ccc(Cl)cc23)C1c1ccc(OCCCn2ccnc2)cc1] | 724 |
| 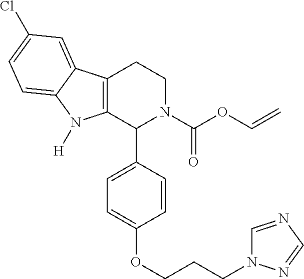 [SMILES: C=COC(=O)N1CCc2c([nH]c3ccc(Cl)cc23)C1c1ccc(OCCCn2cncn2)cc1] | 725 |
| 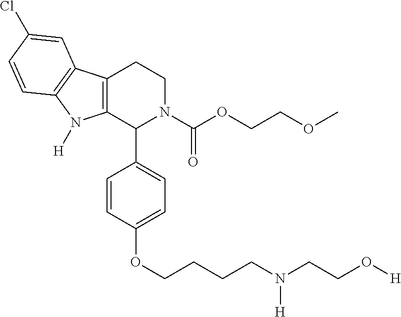 [SMILES: COCCOC(=O)N1CCc2c([nH]c3ccc(Cl)cc23)C1c1ccc(OCCCCNCCO)cc1] | 726 |
| 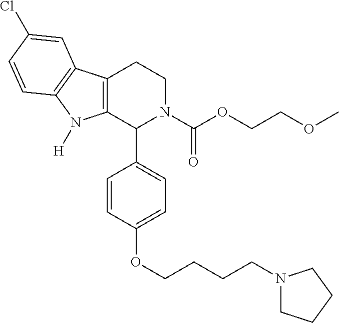 [SMILES: COCCOC(=O)N1CCc2c([nH]c3ccc(Cl)cc23)C1c1ccc(OCCCCN2CCCC2)cc1] | 727 |
| 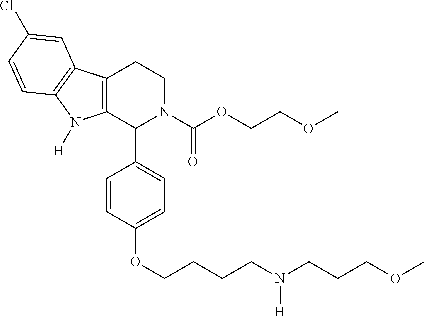 [SMILES: COCCCNCCCCOc1ccc(C2c3[nH]c4ccc(Cl)cc4c3CCN2C(=O)OCCOC)cc1] | 728 |
| 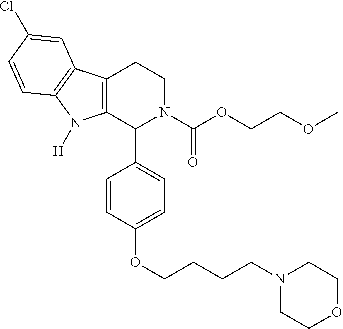 [SMILES: COCCOC(=O)N1CCc2c([nH]c3ccc(Cl)cc23)C1c1ccc(OCCCCN2CCOCC2)cc1] | 729 |
| 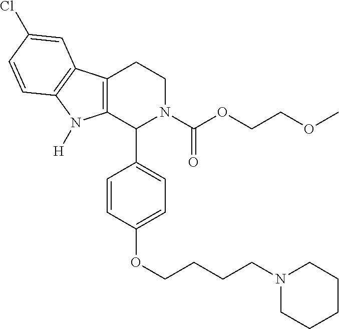 [SMILES: COCCOC(=O)N1CCc2c([nH]c3ccc(Cl)cc23)C1c1ccc(OCCCCN2CCCCC2)cc1] | 730 |
| 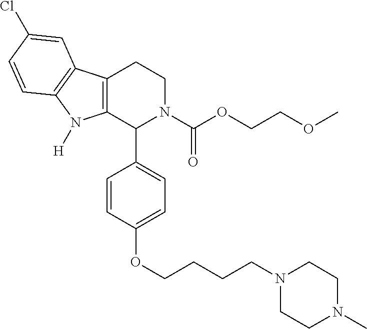 [SMILES: COCCOC(=O)N1CCc2c([nH]c3ccc(Cl)cc23)C1c1ccc(OCCCCN2CCN(C)CC2)cc1] | 731 |
| 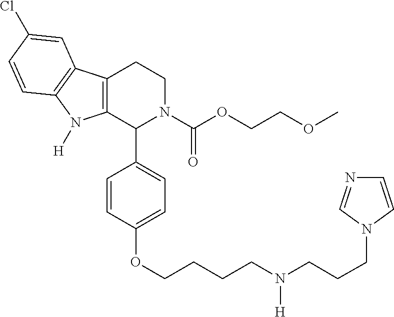 [SMILES: COCCOC(=O)N1CCc2c([nH]c3ccc(Cl)cc23)C1c1ccc(OCCCCNCCCn2ccnc2)cc1] | 732 |
| 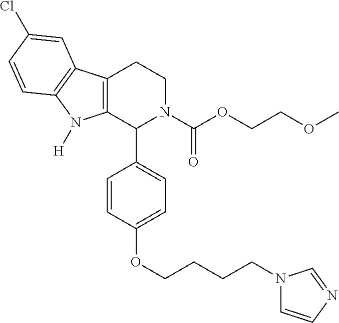 [SMILES: COCCOC(=O)N1CCc2c([nH]c3ccc(Cl)cc23)C1c1ccc(OCCCCn2ccnc2)cc1] | 733 |
| 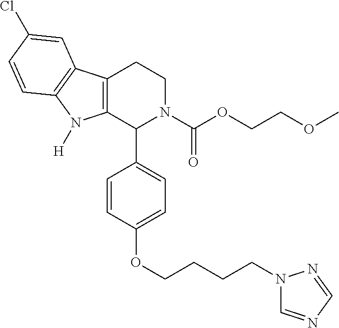 [SMILES: COCCOC(=O)N1CCc2c([nH]c3ccc(Cl)cc23)C1c1ccc(OCCCCn2cncn2)cc1] | 734 |
| 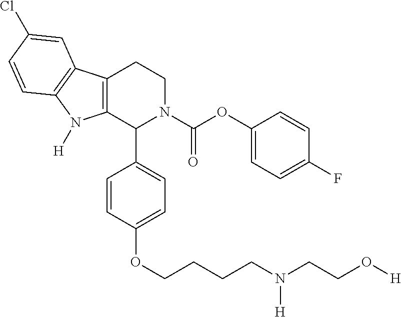 [SMILES: O=C(Oc1ccc(F)cc1)N1CCc2c([nH]c3ccc(Cl)cc23)C1c1ccc(OCCCCNCCO)cc1] | 735 |
| 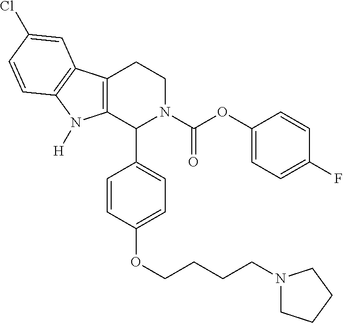 [SMILES: O=C(Oc1ccc(F)cc1)N1CCc2c([nH]c3ccc(Cl)cc23)C1c1ccc(OCCCCN2CCCC2)cc1] | 736 |
| 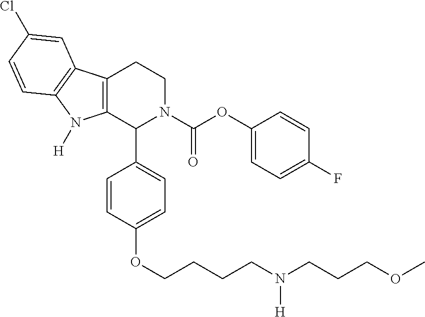 [SMILES: COCCCNCCCCOc1ccc(C2c3[nH]c4ccc(Cl)cc4c3CCN2C(=O)Oc2ccc(F)cc2)cc1] | 737 |
| 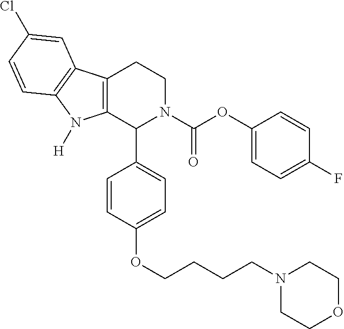 [SMILES: O=C(Oc1ccc(F)cc1)N1CCc2c([nH]c3ccc(Cl)cc23)C1c1ccc(OCCCCN2CCOCC2)cc1] | 738 |
| 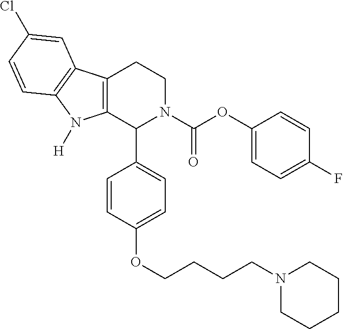 [SMILES: O=C(Oc1ccc(F)cc1)N1CCc2c([nH]c3ccc(Cl)cc23)C1c1ccc(OCCCCN2CCCCC2)cc1] | 739 |
| 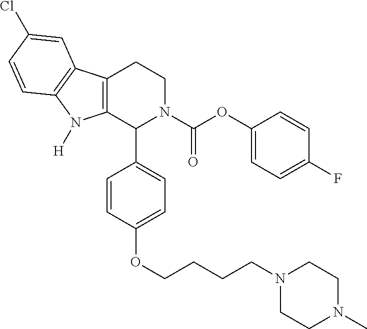 [SMILES: CN1CCN(CCCCOc2ccc(C3c4[nH]c5ccc(Cl)cc5c4CCN3C(=O)Oc3ccc(F)cc3)cc2)CC1] | 740 |
| 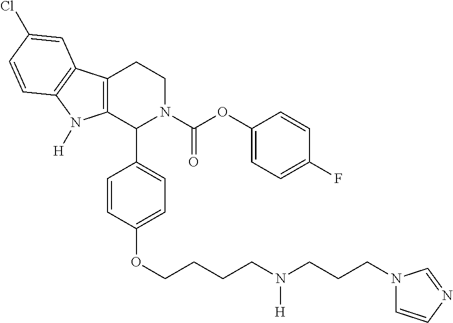 [SMILES: O=C(Oc1ccc(F)cc1)N1CCc2c([nH]c3ccc(Cl)cc23)C1c1ccc(OCCCCNCCCn2ccnc2)cc1] | 741 |
| 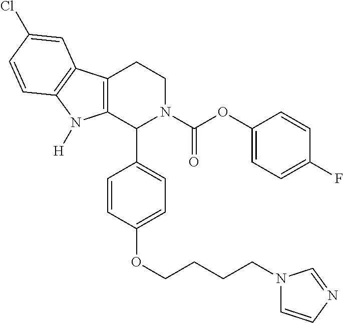 [SMILES: O=C(Oc1ccc(F)cc1)N1CCc2c([nH]c3ccc(Cl)cc23)C1c1ccc(OCCCCn2ccnc2)cc1] | 742 |
| 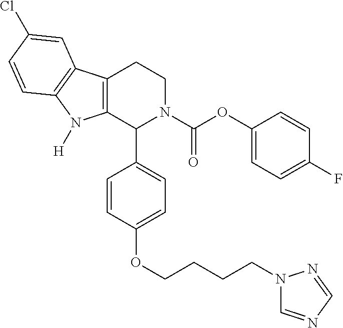 [SMILES: O=C(Oc1ccc(F)cc1)N1CCc2c([nH]c3ccc(Cl)cc23)C1c1ccc(OCCCCn2cncn2)cc1] | 743 |
| 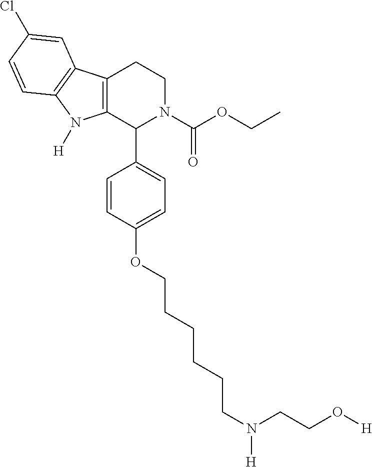 [SMILES: CCOC(=O)N1CCc2c([nH]c3ccc(Cl)cc23)C1c1ccc(OCCCCCCNCCO)cc1] | 744 |
| 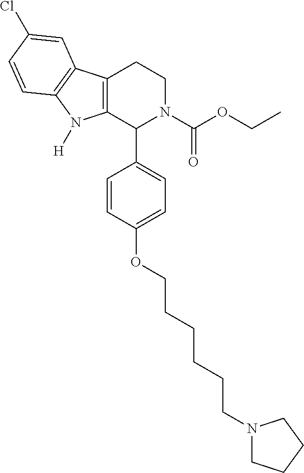 [SMILES: CCOC(=O)N1CCc2c([nH]c3ccc(Cl)cc23)C1c1ccc(OCCCCCCN2CCCC2)cc1] | 745 |
| 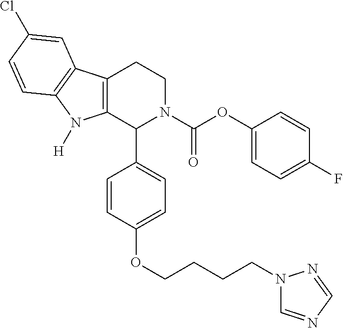 [SMILES: O=C(Oc1ccc(F)cc1)N1CCc2c([nH]c3ccc(Cl)cc23)C1c1ccc(OCCCCn2cncn2)cc1] | 746 |
| 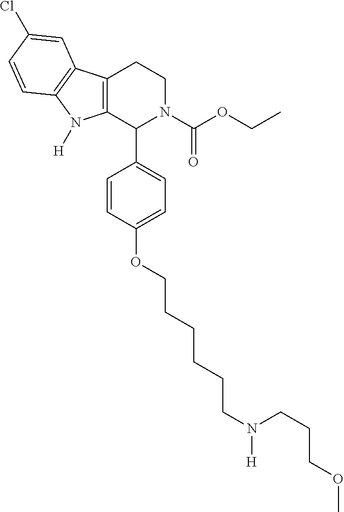 [SMILES: CCOC(=O)N1CCc2c([nH]c3ccc(Cl)cc23)C1c1ccc(OCCCCCCNCCCOC)cc1] | 747 |
| 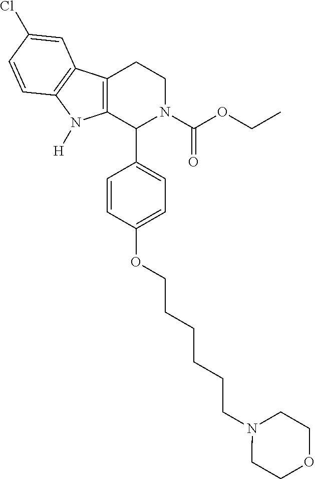 [SMILES: CCOC(=O)N1CCc2c([nH]c3ccc(Cl)cc23)C1c1ccc(OCCCCCCN2CCOCC2)cc1] | 748 |
| 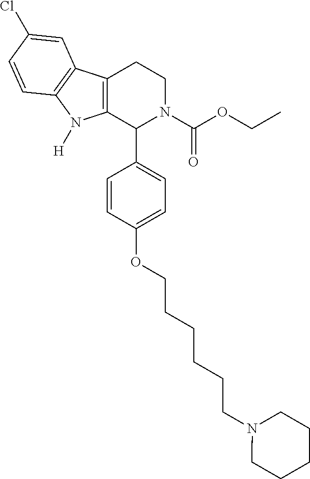 [SMILES: CCOC(=O)N1CCc2c([nH]c3ccc(Cl)cc23)C1c1ccc(OCCCCCCN2CCCCC2)cc1] | 749 |
| 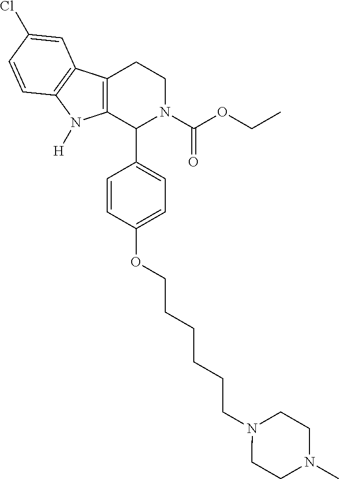 [SMILES: CCOC(=O)N1CCc2c([nH]c3ccc(Cl)cc23)C1c1ccc(OCCCCCCN2CCN(C)CC2)cc1] | 750 |
| 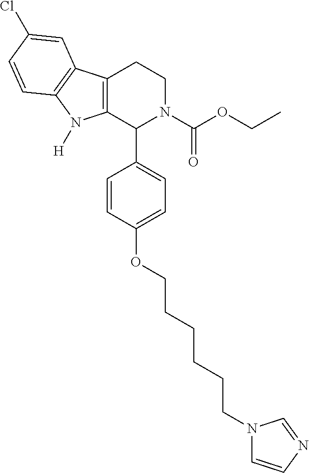 [SMILES: CCOC(=O)N1CCc2c([nH]c3ccc(Cl)cc23)C1c1ccc(OCCCCCCn2ccnc2)cc1] | 751 |
| 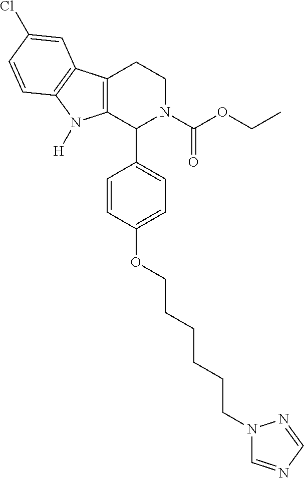 [SMILES: CCOC(=O)N1CCc2c([nH]c3ccc(Cl)cc23)C1c1ccc(OCCCCCCn2cncn2)cc1] | 752 |
| 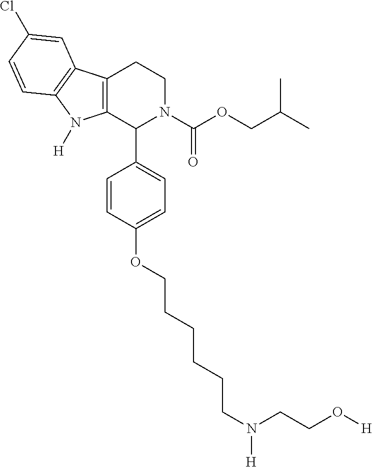 [SMILES: CC(C)COC(=O)N1CCc2c([nH]c3ccc(Cl)cc23)C1c1ccc(OCCCCCCNCCO)cc1] | 753 |
| 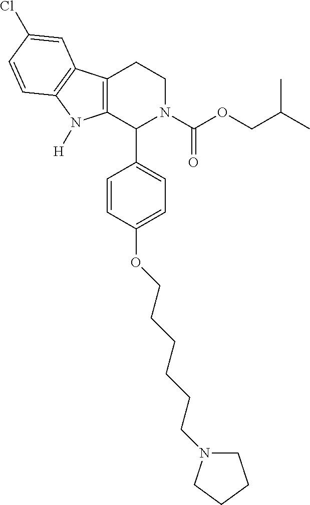 [SMILES: CC(C)COC(=O)N1CCc2c([nH]c3ccc(Cl)cc23)C1c1ccc(OCCCCCCN2CCCC2)cc1] | 754 |
| 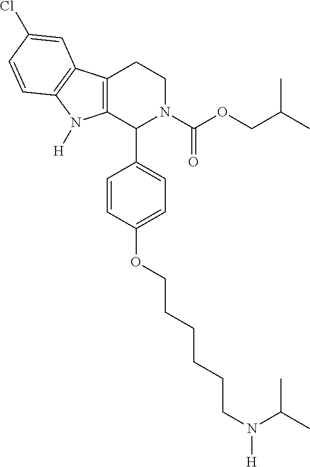 [SMILES: CC(C)COC(=O)N1CCc2c([nH]c3ccc(Cl)cc23)C1c1ccc(OCCCCCCNC(C)C)cc1] | 755 |
| 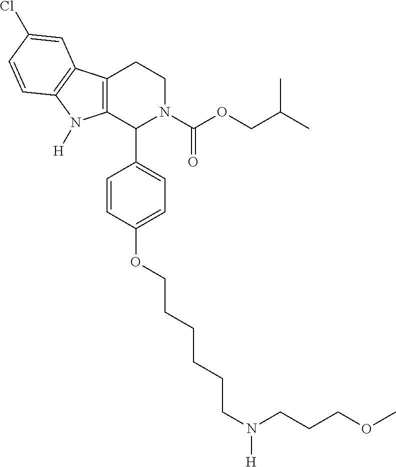 [SMILES: COCCCNCCCCCCOc1ccc(C2c3[nH]c4ccc(Cl)cc4c3CCN2C(=O)OCC(C)C)cc1] | 756 |
| 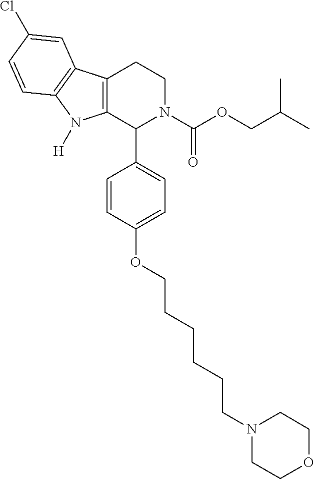 [SMILES: CC(C)COC(=O)N1CCc2c([nH]c3ccc(Cl)cc23)C1c1ccc(OCCCCCCN2CCOCC2)cc1] | 757 |
| 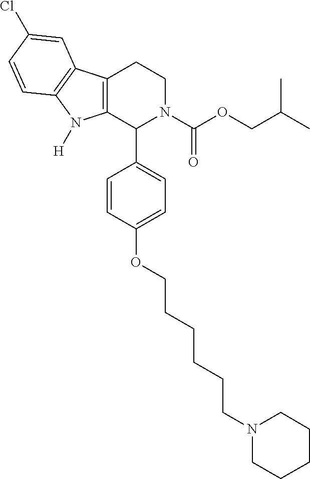 [SMILES: CC(C)COC(=O)N1CCc2c([nH]c3ccc(Cl)cc23)C1c1ccc(OCCCCCCN2CCCCC2)cc1] | 758 |
| 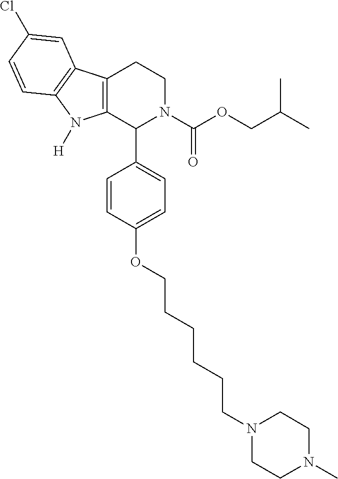 [SMILES: CC(C)COC(=O)N1CCc2c([nH]c3ccc(Cl)cc23)C1c1ccc(OCCCCCCN2CCN(C)CC2)cc1] | 759 |
| 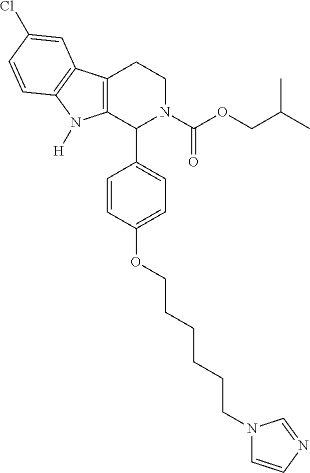 [SMILES: CC(C)COC(=O)N1CCc2c([nH]c3ccc(Cl)cc23)C1c1ccc(OCCCCCCn2ccnc2)cc1] | 760 |
| 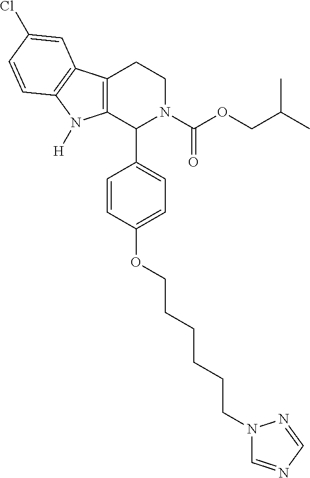 [SMILES: CC(C)COC(=O)N1CCc2c([nH]c3ccc(Cl)cc23)C1c1ccc(OCCCCCCn2cncn2)cc1] | 761 |
| 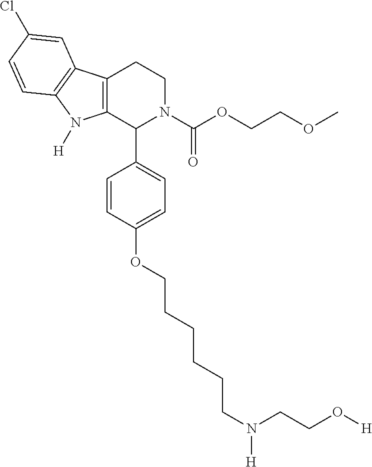 [SMILES: COCCOC(=O)N1CCc2c([nH]c3ccc(Cl)cc23)C1c1ccc(OCCCCCCNCCO)cc1] | 762 |
| 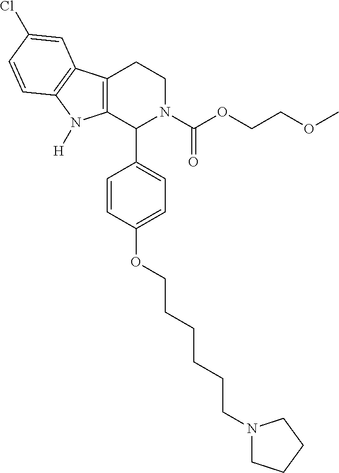 [SMILES: COCCOC(=O)N1CCc2c([nH]c3ccc(Cl)cc23)C1c1ccc(OCCCCCCN2CCCC2)cc1] | 763 |
| 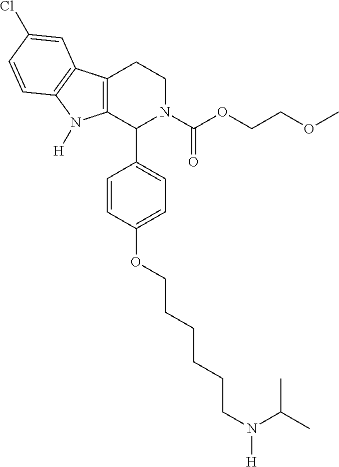 [SMILES: COCCOC(=O)N1CCc2c([nH]c3ccc(Cl)cc23)C1c1ccc(OCCCCCCNC(C)C)cc1] | 764 |
| 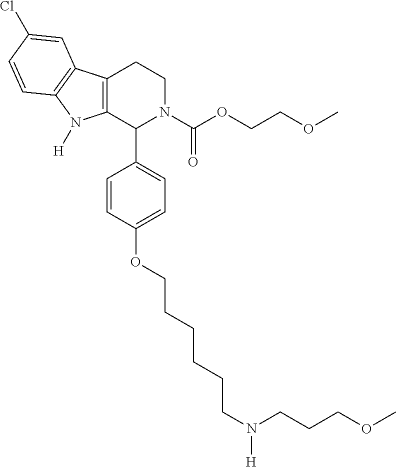 [SMILES: COCCCNCCCCCCOc1ccc(C2c3[nH]c4ccc(Cl)cc4c3CCN2C(=O)OCCOC)cc1] | 765 |
| 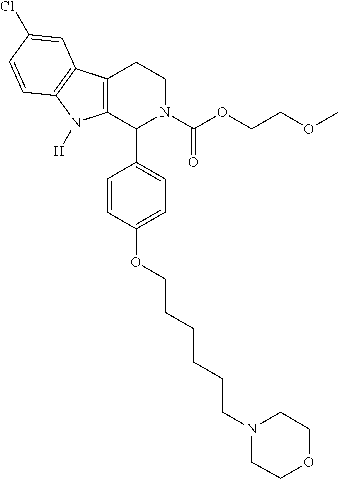 [SMILES: COCCOC(=O)N1CCc2c([nH]c3ccc(Cl)cc23)C1c1ccc(OCCCCCCN2CCOCC2)cc1] | 766 |
| 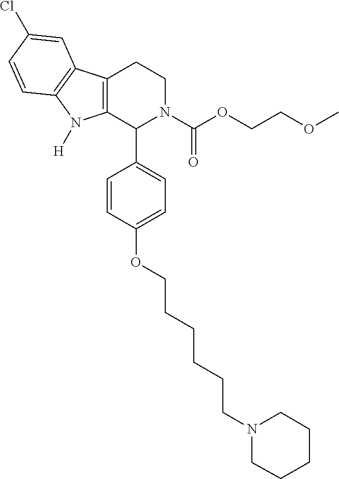 [SMILES: COCCOC(=O)N1CCc2c([nH]c3ccc(Cl)cc23)C1c1ccc(OCCCCCCN2CCCCC2)cc1] | 767 |
| 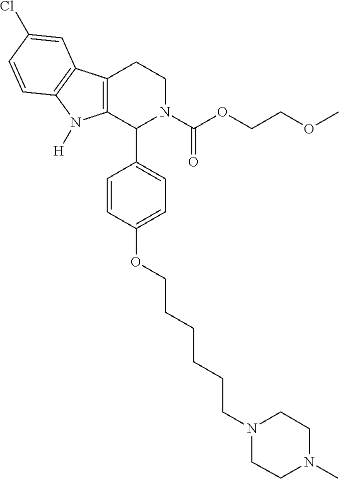 [SMILES: COCCOC(=O)N1CCc2c([nH]c3ccc(Cl)cc23)C1c1ccc(OCCCCCCN2CCN(C)CC2)cc1] | 768 |
| 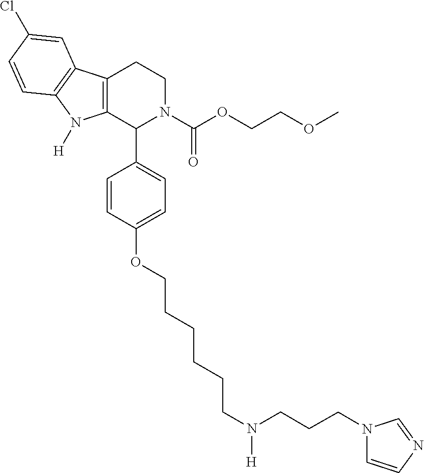 [SMILES: COCCOC(=O)N1CCc2c([nH]c3ccc(Cl)cc23)C1c1ccc(OCCCCCCNCCCn2ccnc2)cc1] | 769 |
| 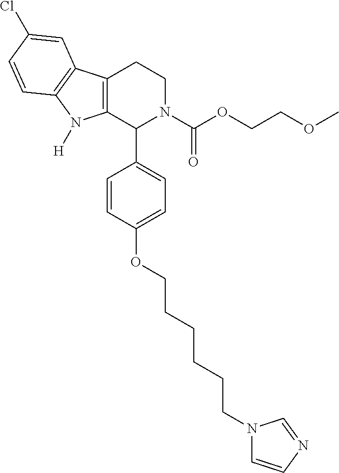 [SMILES: COCCOC(=O)N1CCc2c([nH]c3ccc(Cl)cc23)C1c1ccc(OCCCCCCn2ccnc2)cc1] | 770 |
| 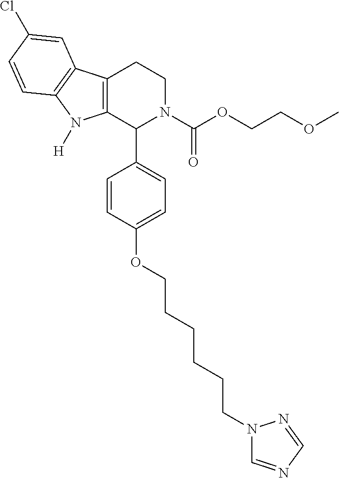 [SMILES: COCCOC(=O)N1CCc2c([nH]c3ccc(Cl)cc23)C1c1ccc(OCCCCCCn2cncn2)cc1] | 771 |
| 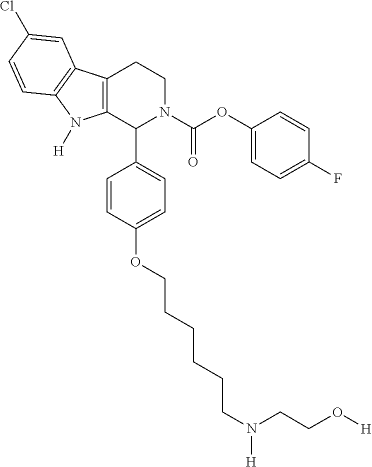 [SMILES: O=C(Oc1ccc(F)cc1)N1CCc2c([nH]c3ccc(Cl)cc23)C1c1ccc(OCCCCCCNCCO)cc1] | 772 |
| 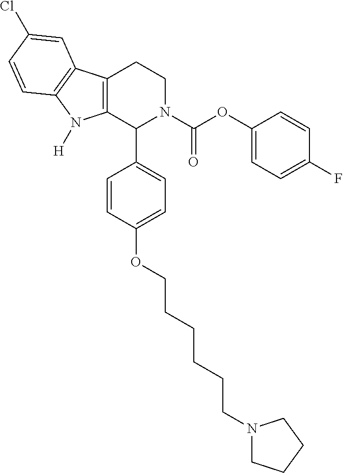 [SMILES: O=C(Oc1ccc(F)cc1)N1CCc2c([nH]c3ccc(Cl)cc23)C1c1ccc(OCCCCCCN2CCCC2)cc1] | 773 |
| 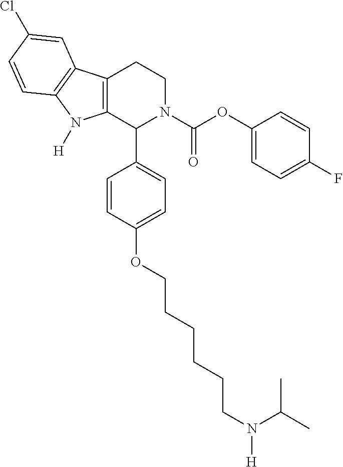 [SMILES: CC(C)NCCCCCCOc1ccc(C2c3[nH]c4ccc(Cl)cc4c3CCN2C(=O)Oc2ccc(F)cc2)cc1] | 774 |
| 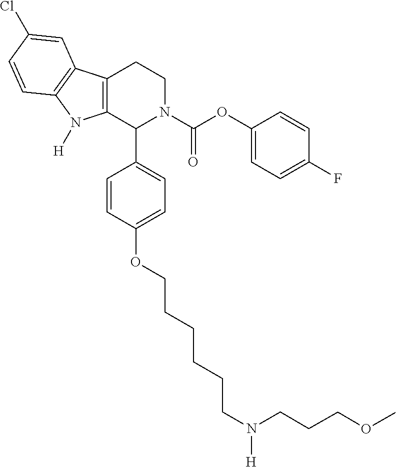 [SMILES: COCCCNCCCCCCOc1ccc(C2c3[nH]c4ccc(Cl)cc4c3CCN2C(=O)Oc2ccc(F)cc2)cc1] | 775 |
| 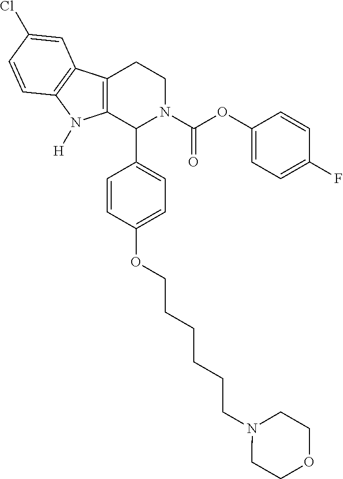 [SMILES: O=C(Oc1ccc(F)cc1)N1CCc2c([nH]c3ccc(Cl)cc23)C1c1ccc(OCCCCCCN2CCOCC2)cc1] | 776 |
| 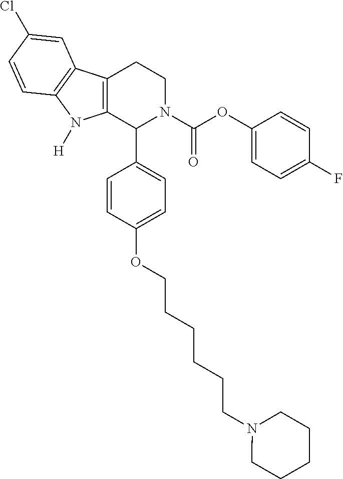 [SMILES: O=C(Oc1ccc(F)cc1)N1CCc2c([nH]c3ccc(Cl)cc23)C1c1ccc(OCCCCCCN2CCCCC2)cc1] | 777 |
| 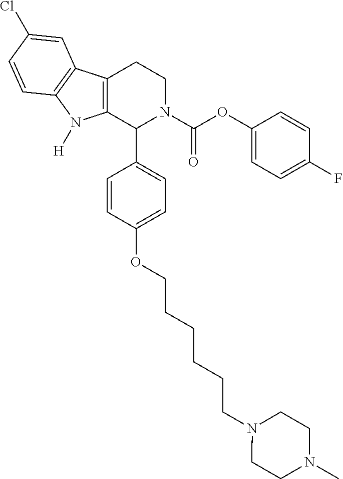 [SMILES: CN1CCN(CCCCCCOc2ccc(C3c4[nH]c5ccc(Cl)cc5c4CCN3C(=O)Oc3ccc(F)cc3)cc2)CC1] | 778 |
| 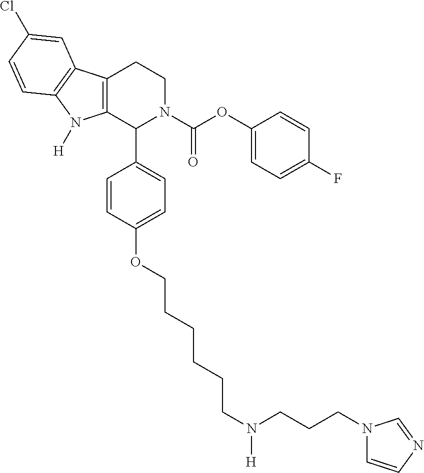 [SMILES: O=C(Oc1ccc(F)cc1)N1CCc2c([nH]c3ccc(Cl)cc23)C1c1ccc(OCCCCCCNCCCn2ccnc2)cc1] | 779 |
| 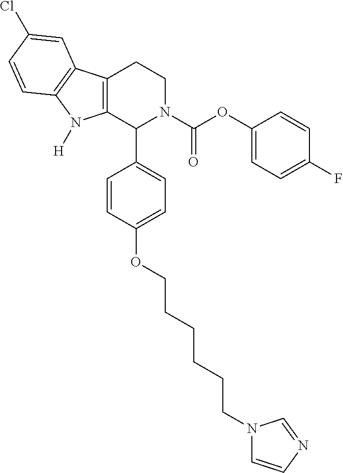 [SMILES: O=C(Oc1ccc(F)cc1)N1CCc2c([nH]c3ccc(Cl)cc23)C1c1ccc(OCCCCCCn2ccnc2)cc1] | 780 |
| 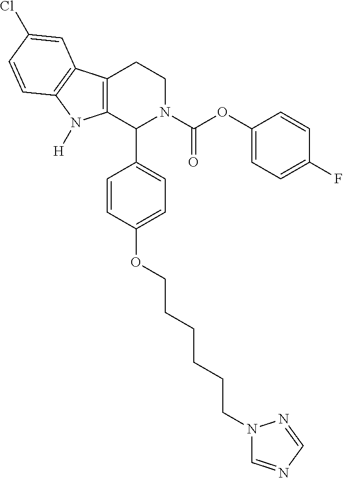 [SMILES: O=C(Oc1ccc(F)cc1)N1CCc2c([nH]c3ccc(Cl)cc23)C1c1ccc(OCCCCCCn2cncn2)cc1] | 781 |
| 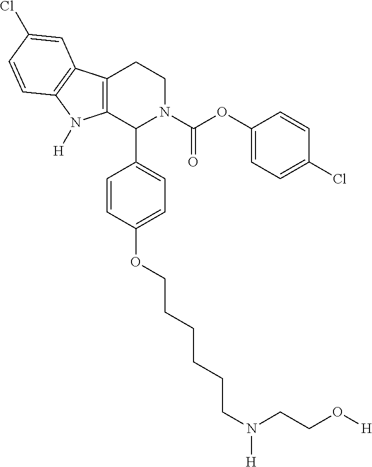 [SMILES: O=C(Oc1ccc(Cl)cc1)N1CCc2c([nH]c3ccc(Cl)cc23)C1c1ccc(OCCCCCCNCCO)cc1] | 782 |
| 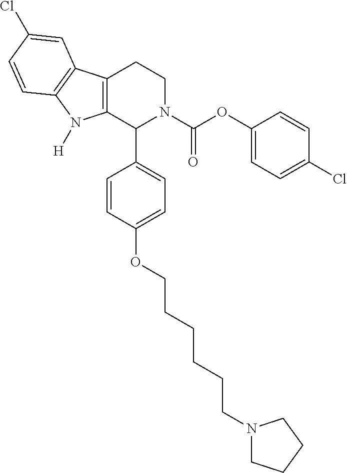 [SMILES: O=C(Oc1ccc(Cl)cc1)N1CCc2c([nH]c3ccc(Cl)cc23)C1c1ccc(OCCCCCCN2CCCC2)cc1] | 783 |
| 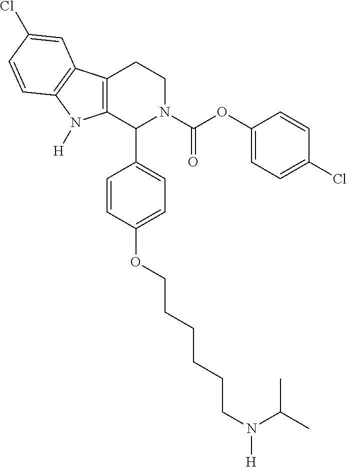 [SMILES: CC(C)NCCCCCCOc1ccc(C2c3[nH]c4ccc(Cl)cc4c3CCN2C(=O)Oc2ccc(Cl)cc2)cc1] | 784 |
| 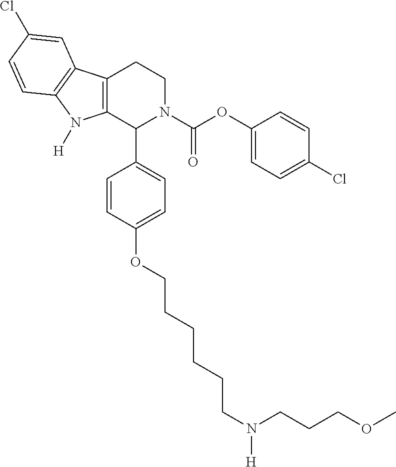 [SMILES: COCCCNCCCCCCOc1ccc(C2c3[nH]c4ccc(Cl)cc4c3CCN2C(=O)Oc2ccc(Cl)cc2)cc1] | 785 |
| 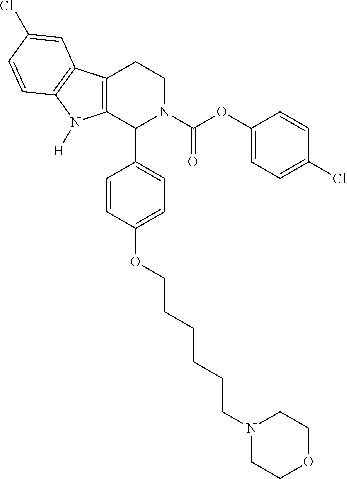 [SMILES: O=C(Oc1ccc(Cl)cc1)N1CCc2c([nH]c3ccc(Cl)cc23)C1c1ccc(OCCCCCCN2CCOCC2)cc1] | 786 |
| 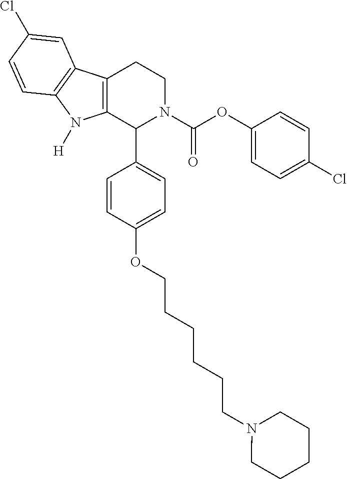 [SMILES: O=C(Oc1ccc(Cl)cc1)N1CCc2c([nH]c3ccc(Cl)cc23)C1c1ccc(OCCCCCCN2CCCCC2)cc1] | 787 |
| 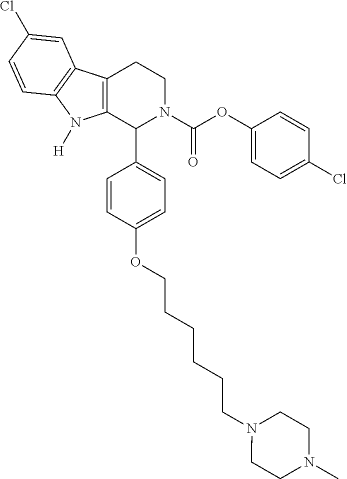 [SMILES: CN1CCN(CCCCCCOc2ccc(C3c4[nH]c5ccc(Cl)cc5c4CCN3C(=O)Oc3ccc(Cl)cc3)cc2)CC1] | 788 |
| 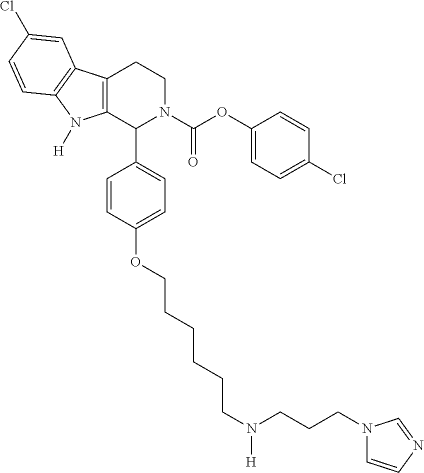 [SMILES: O=C(Oc1ccc(Cl)cc1)N1CCc2c([nH]c3ccc(Cl)cc23)C1c1ccc(OCCCCCCNCCCn2ccnc2)cc1] | 789 |
| 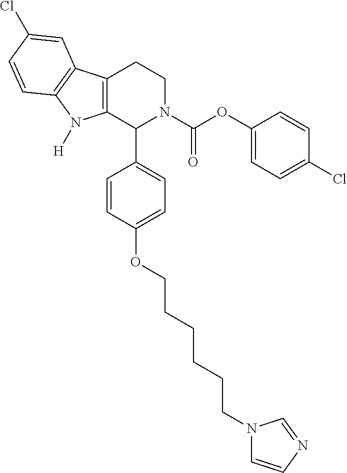 [SMILES: O=C(Oc1ccc(Cl)cc1)N1CCc2c([nH]c3ccc(Cl)cc23)C1c1ccc(OCCCCCCn2ccnc2)cc1] | 790 |
| 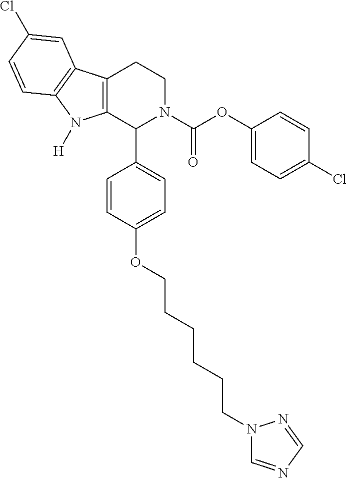 [SMILES: O=C(Oc1ccc(Cl)cc1)N1CCc2c([nH]c3ccc(Cl)cc23)C1c1ccc(OCCCCCCn2cncn2)cc1] | 791 |
| 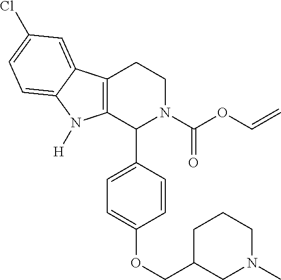 [SMILES: C=COC(=O)N1CCc2c([nH]c3ccc(Cl)cc23)C1c1ccc(OCC2CCCN(C)C2)cc1] | 792 |
| 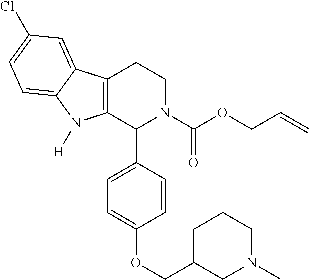 [SMILES: C=CCOC(=O)N1CCc2c([nH]c3ccc(Cl)cc23)C1c1ccc(OCC2CCCN(C)C2)cc1] | 793 |
| 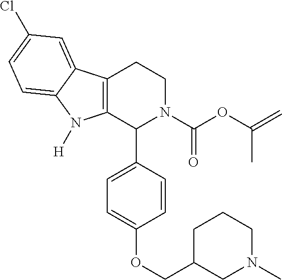 [SMILES: C=C(C)OC(=O)N1CCc2c([nH]c3ccc(Cl)cc23)C1c1ccc(OCC2CCCN(C)C2)cc1] | 794 |
| 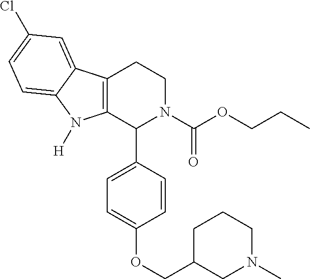 [SMILES: CCCOC(=O)N1CCc2c([nH]c3ccc(Cl)cc23)C1c1ccc(OCC2CCCN(C)C2)cc1] | 795 |
| 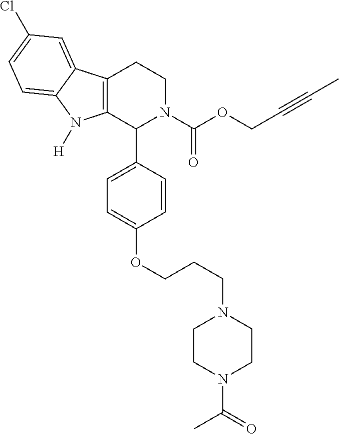 [SMILES: CC#CCOC(=O)N1CCc2c([nH]c3ccc(Cl)cc23)C1c1ccc(OCCCN2CCN(C(C)=O)CC2)cc1] | 796 |
| 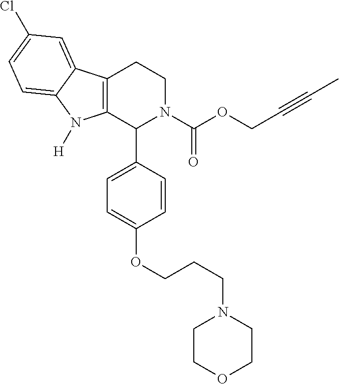 [SMILES: CC#CCOC(=O)N1CCc2c([nH]c3ccc(Cl)cc23)C1c1ccc(OCCCN2CCOCC2)cc1] | 797 |
| 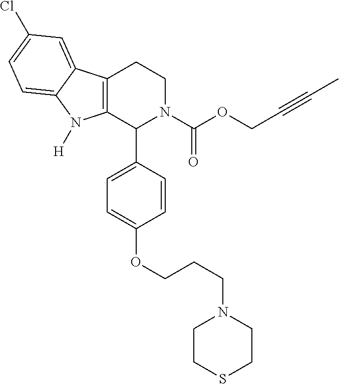 [SMILES: CC#CCOC(=O)N1CCc2c([nH]c3ccc(Cl)cc23)C1c1ccc(OCCCN2CCSCC2)cc1] | 798 |
| 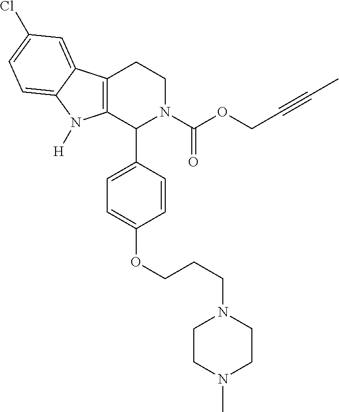 [SMILES: CC#CCOC(=O)N1CCc2c([nH]c3ccc(Cl)cc23)C1c1ccc(OCCCN2CCN(C)CC2)cc1] | 799 |
| 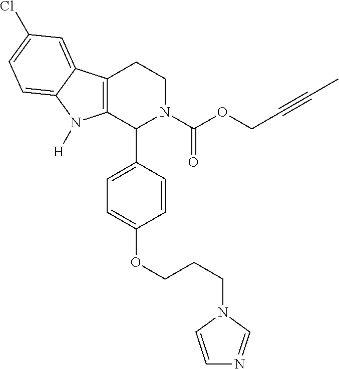 [SMILES: CC#CCOC(=O)N1CCc2c([nH]c3ccc(Cl)cc23)C1c1ccc(OCCCn2ccnc2)cc1] | 800 |
| 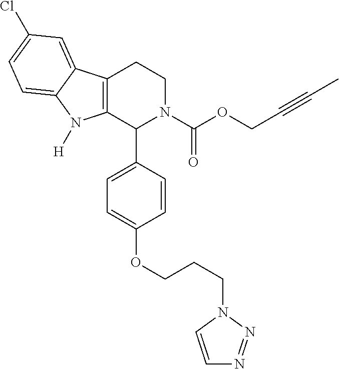 [SMILES: CC#CCOC(=O)N1CCc2c([nH]c3ccc(Cl)cc23)C1c1ccc(OCCCn2ccnn2)cc1] | 801 |
| 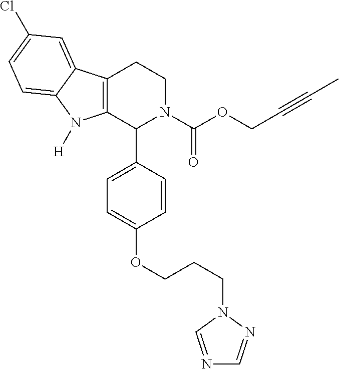 [SMILES: CC#CCOC(=O)N1CCc2c([nH]c3ccc(Cl)cc23)C1c1ccc(OCCCn2cncn2)cc1] | 802 |
| 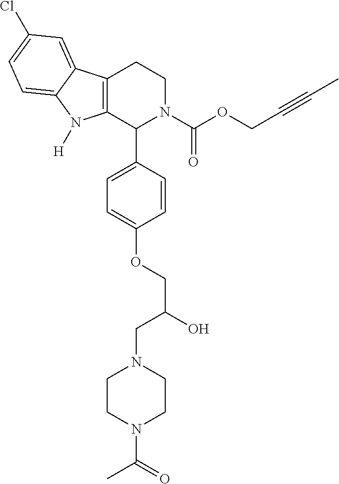 [SMILES: CC#CCOC(=O)N1CCc2c([nH]c3ccc(Cl)cc23)C1c1ccc(OCC(O)CN2CCN(C(C)=O)CC2)cc1] | 803 |
| 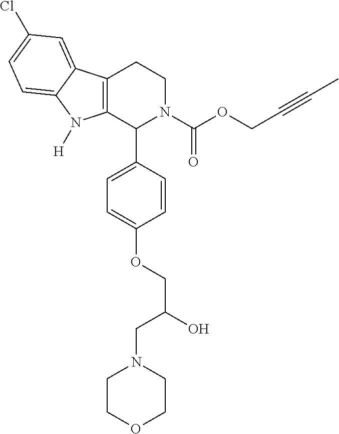 [SMILES: CC#CCOC(=O)N1CCc2c([nH]c3ccc(Cl)cc23)C1c1ccc(OCC(O)CN2CCOCC2)cc1] | 804 |
| 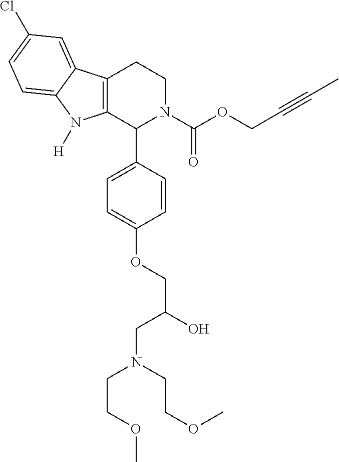 [SMILES: CC#CCOC(=O)N1CCc2c([nH]c3ccc(Cl)cc23)C1c1ccc(OCC(O)CN(CCOC)CCOC)cc1] | 805 |
| 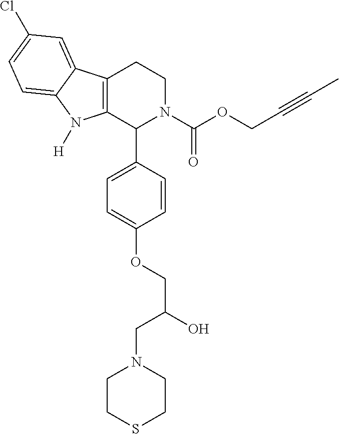 [SMILES: CC#CCOC(=O)N1CCc2c([nH]c3ccc(Cl)cc23)C1c1ccc(OCC(O)CN2CCSCC2)cc1] | 806 |
| 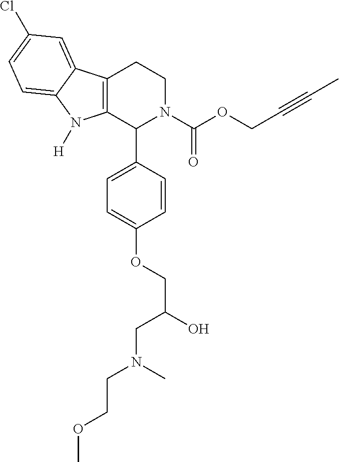 [SMILES: CC#CCOC(=O)N1CCc2c([nH]c3ccc(Cl)cc23)C1c1ccc(OCC(O)CN(C)CCOC)cc1] | 807 |
| 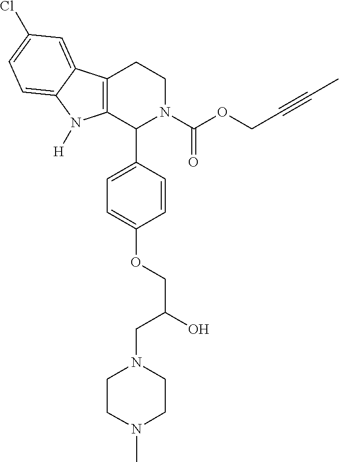 [SMILES: CC#CCOC(=O)N1CCc2c([nH]c3ccc(Cl)cc23)C1c1ccc(OCC(O)CN2CCN(C)CC2)cc1] | 808 |
| 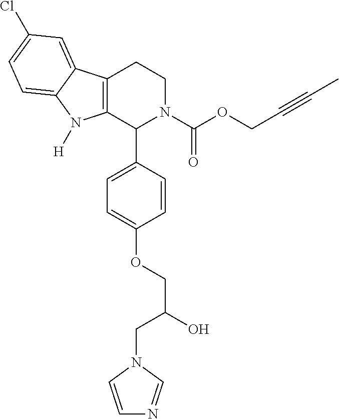 [SMILES: CC#CCOC(=O)N1CCc2c([nH]c3ccc(Cl)cc23)C1c1ccc(OCC(O)Cn2ccnc2)cc1] | 809 |
| 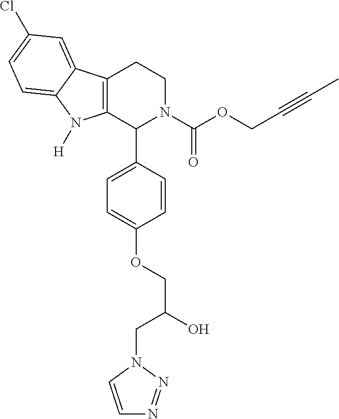 [SMILES: CC#CCOC(=O)N1CCc2c([nH]c3ccc(Cl)cc23)C1c1ccc(OCC(O)Cn2ccnn2)cc1] | 810 |
| 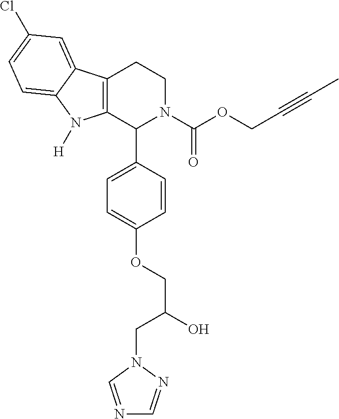 [SMILES: CC#CCOC(=O)N1CCc2c([nH]c3ccc(Cl)cc23)C1c1ccc(OCC(O)Cn2cncn2)cc1] | 811 |
| 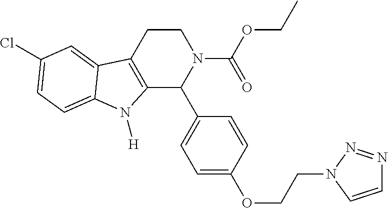 [SMILES: CCOC(=O)N1CCc2c([nH]c3ccc(Cl)cc23)C1c1ccc(OCCn2ccnn2)cc1] | 812 |
| 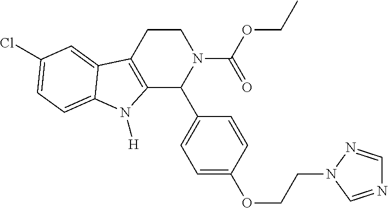 [SMILES: CCOC(=O)N1CCc2c([nH]c3ccc(Cl)cc23)C1c1ccc(OCCn2cncn2)cc1] | 813 |
| 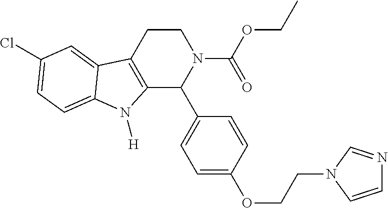 [SMILES: CCOC(=O)N1CCc2c([nH]c3ccc(Cl)cc23)C1c1ccc(OCCn2ccnc2)cc1] | 814 |
| 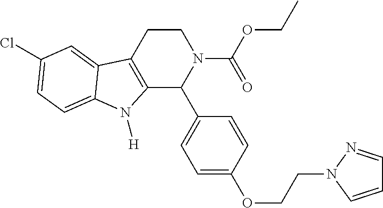 [SMILES: CCOC(=O)N1CCc2c([nH]c3ccc(Cl)cc23)C1c1ccc(OCCn2cccn2)cc1] | 815 |
| 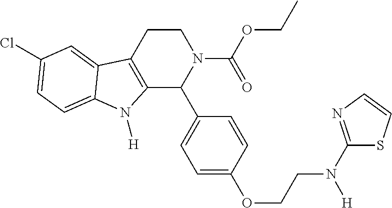 [SMILES: CCOC(=O)N1CCc2c([nH]c3ccc(Cl)cc23)C1c1ccc(OCCNc2nccs2)cc1] | 816 |
| 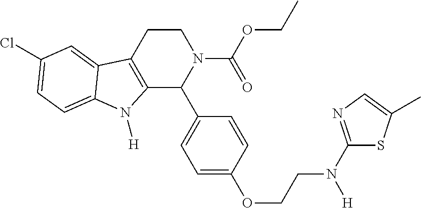 [SMILES: CCOC(=O)N1CCc2c([nH]c3ccc(Cl)cc23)C1c1ccc(OCCNc2ncc(C)s2)cc1] | 817 |
| 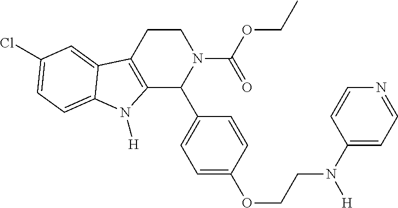 [SMILES: CCOC(=O)N1CCc2c([nH]c3ccc(Cl)cc23)C1c1ccc(OCCNc2ccncc2)cc1] | 818 |
| 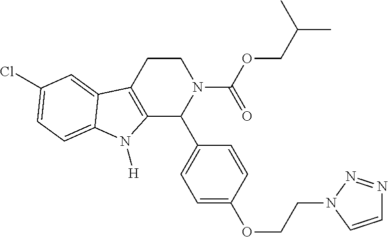 [SMILES: CC(C)COC(=O)N1CCc2c([nH]c3ccc(Cl)cc23)C1c1ccc(OCCn2ccnn2)cc1] | 819 |
| 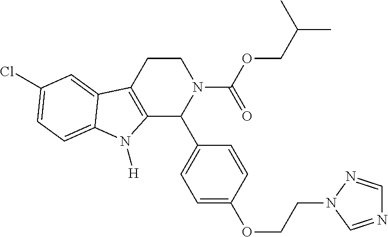 [SMILES: CC(C)COC(=O)N1CCc2c([nH]c3ccc(Cl)cc23)C1c1ccc(OCCn2cncn2)cc1] | 820 |
| 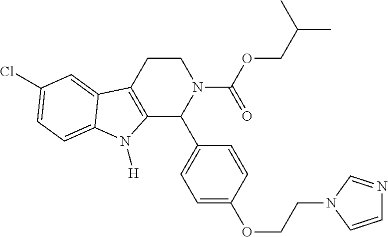 [SMILES: CC(C)COC(=O)N1CCc2c([nH]c3ccc(Cl)cc23)C1c1ccc(OCCn2ccnc2)cc1] | 821 |
| 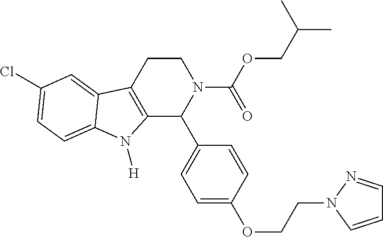 [SMILES: CC(C)COC(=O)N1CCc2c([nH]c3ccc(Cl)cc23)C1c1ccc(OCCn2cccn2)cc1] | 822 |
| 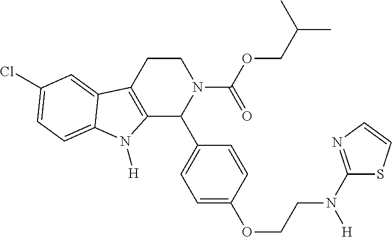 [SMILES: CC(C)COC(=O)N1CCc2c([nH]c3ccc(Cl)cc23)C1c1ccc(OCCNc2nccs2)cc1] | 823 |
| 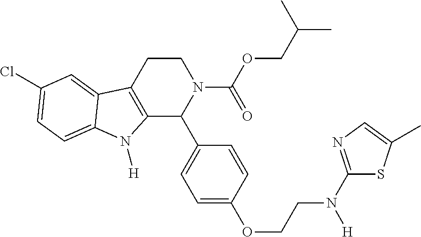 [SMILES: Cc1cnc(NCCOc2ccc(C3c4[nH]c5ccc(Cl)cc5c4CCN3C(=O)OCC(C)C)cc2)s1] | 824 |
| 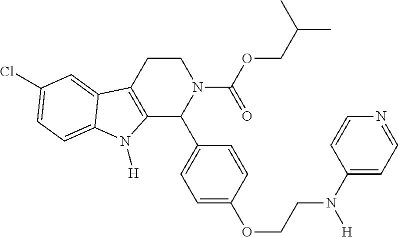 [SMILES: CC(C)COC(=O)N1CCc2c([nH]c3ccc(Cl)cc23)C1c1ccc(OCCNc2ccncc2)cc1] | 825 |
| 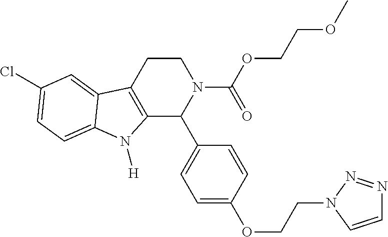 [SMILES: COCCOC(=O)N1CCc2c([nH]c3ccc(Cl)cc23)C1c1ccc(OCCn2ccnn2)cc1] | 826 |
| 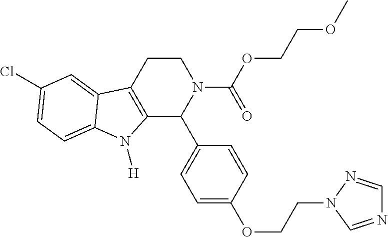 [SMILES: COCCOC(=O)N1CCc2c([nH]c3ccc(Cl)cc23)C1c1ccc(OCCn2cncn2)cc1] | 827 |
| 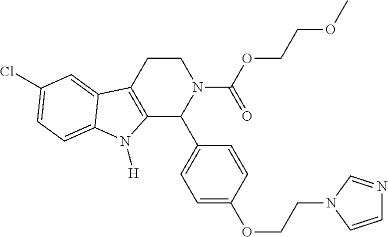 [SMILES: COCCOC(=O)N1CCc2c([nH]c3ccc(Cl)cc23)C1c1ccc(OCCn2ccnc2)cc1] | 828 |
| 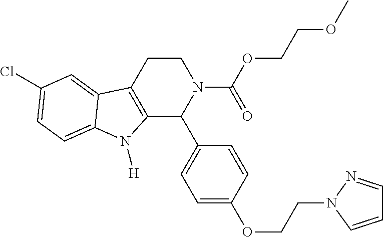 [SMILES: COCCOC(=O)N1CCc2c([nH]c3ccc(Cl)cc23)C1c1ccc(OCCn2cccn2)cc1] | 829 |
| 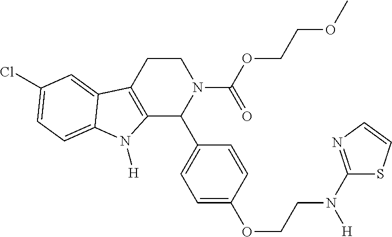 [SMILES: COCCOC(=O)N1CCc2c([nH]c3ccc(Cl)cc23)C1c1ccc(OCCNc2nccs2)cc1] | 830 |
| 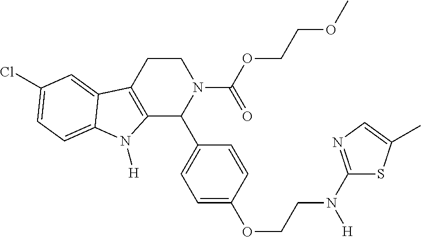 [SMILES: COCCOC(=O)N1CCc2c([nH]c3ccc(Cl)cc23)C1c1ccc(OCCNc2ncc(C)s2)cc1] | 831 |
| 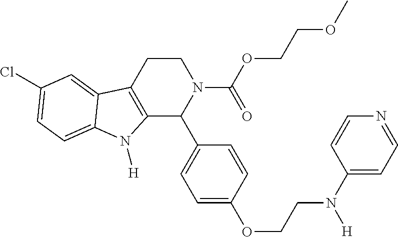 [SMILES: COCCOC(=O)N1CCc2c([nH]c3ccc(Cl)cc23)C1c1ccc(OCCNc2ccncc2)cc1] | 832 |
| 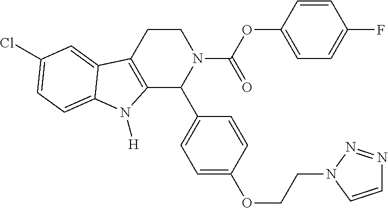 [SMILES: O=C(Oc1ccc(F)cc1)N1CCc2c([nH]c3ccc(Cl)cc23)C1c1ccc(OCCn2ccnn2)cc1] | 833 |
| 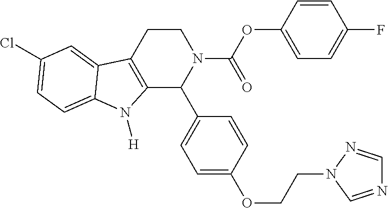 [SMILES: O=C(Oc1ccc(F)cc1)N1CCc2c([nH]c3ccc(Cl)cc23)C1c1ccc(OCCn2cncn2)cc1] | 834 |
| 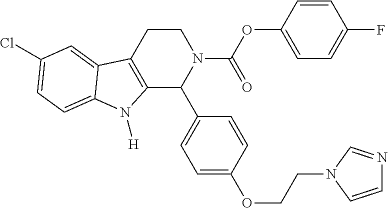 [SMILES: O=C(Oc1ccc(F)cc1)N1CCc2c([nH]c3ccc(Cl)cc23)C1c1ccc(OCCn2ccnc2)cc1] | 835 |
| 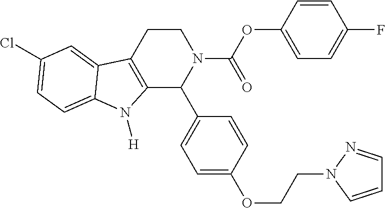 [SMILES: O=C(Oc1ccc(F)cc1)N1CCc2c([nH]c3ccc(Cl)cc23)C1c1ccc(OCCn2cccn2)cc1] | 836 |
| 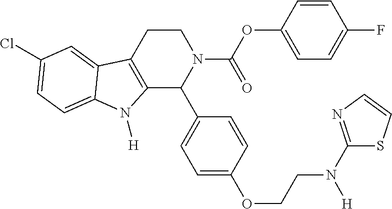 [SMILES: O=C(Oc1ccc(F)cc1)N1CCc2c([nH]c3ccc(Cl)cc23)C1c1ccc(OCCNc2nccs2)cc1] | 837 |
| 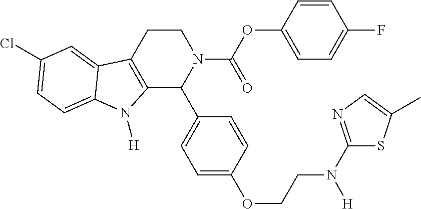 [SMILES: Cc1cnc(NCCOc2ccc(C3c4[nH]c5ccc(Cl)cc5c4CCN3C(=O)Oc3ccc(F)cc3)cc2)s1] | 838 |
| 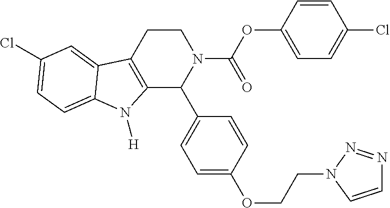 [SMILES: O=C(Oc1ccc(Cl)cc1)N1CCc2c([nH]c3ccc(Cl)cc23)C1c1ccc(OCCn2ccnn2)cc1] | 839 |
| 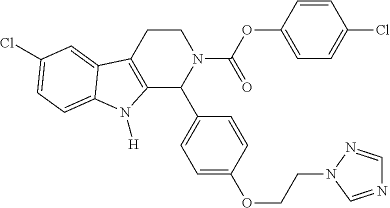 [SMILES: O=C(Oc1ccc(Cl)cc1)N1CCc2c([nH]c3ccc(Cl)cc23)C1c1ccc(OCCn2cncn2)cc1] | 840 |
| 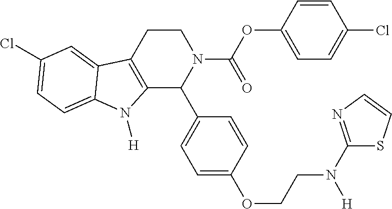 [SMILES: O=C(Oc1ccc(Cl)cc1)N1CCc2c([nH]c3ccc(Cl)cc23)C1c1ccc(OCCNc2nccs2)cc1] | 841 |
| 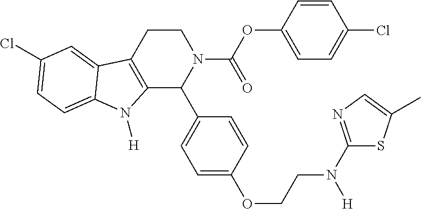 [SMILES: Cc1cnc(NCCOc2ccc(C3c4[nH]c5ccc(Cl)cc5c4CCN3C(=O)Oc3ccc(Cl)cc3)cc2)s1] | 842 |
| 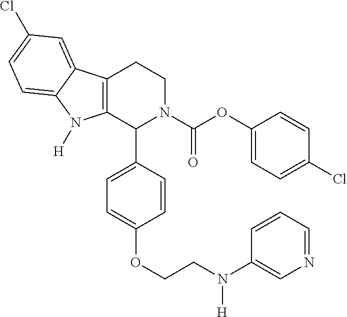 [SMILES: O=C(Oc1ccc(Cl)cc1)N1CCc2c([nH]c3ccc(Cl)cc23)C1c1ccc(OCCNc2cccnc2)cc1] | 843 |
| 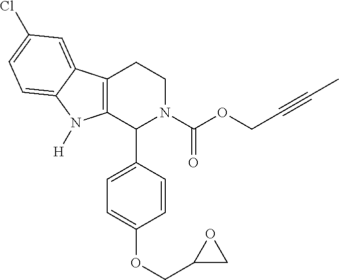 [SMILES: CC#CCOC(=O)N1CCc2c([nH]c3ccc(Cl)cc23)C1c1ccc(OCC2CO2)cc1] | 844 |
| 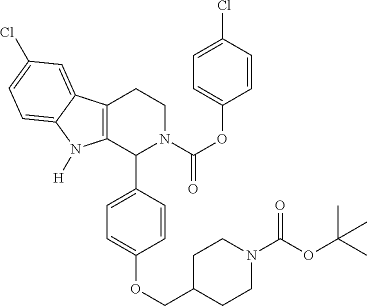 [SMILES: CC(C)(C)OC(=O)N1CCC(COc2ccc(C3c4[nH]c5ccc(Cl)cc5c4CCN3C(=O)Oc3ccc(Cl)cc3)cc2)CC1] | 845 |
| 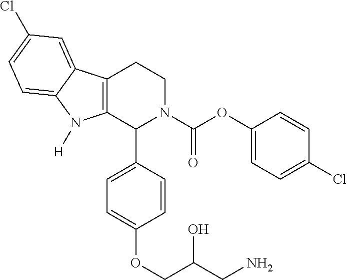 [SMILES: NCC(O)COc1ccc(C2c3[nH]c4ccc(Cl)cc4c3CCN2C(=O)Oc2ccc(Cl)cc2)cc1] | 846 |
| 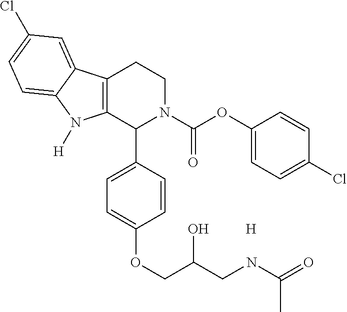 | 847 |
| 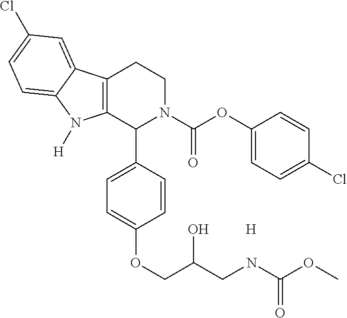 | 848 |
| 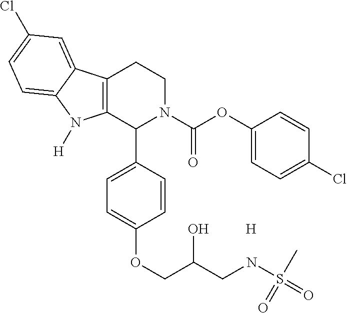 | 849 |
| 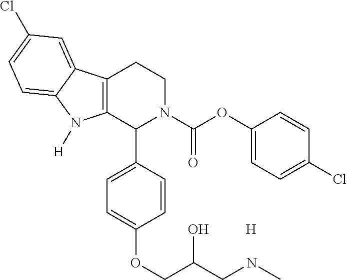 | 850 |
| 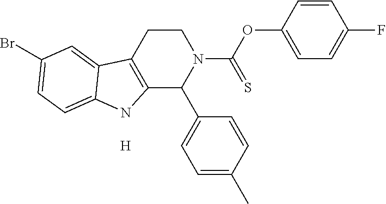 | 851 |
| 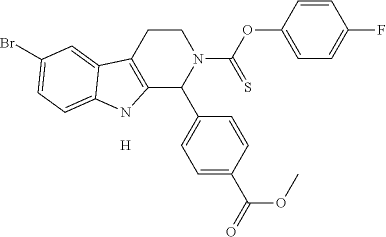 | 852 |
| 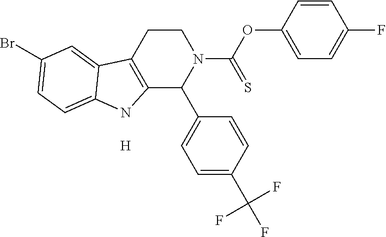 | 853 |
| 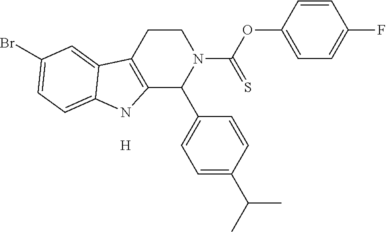 | 854 |
| 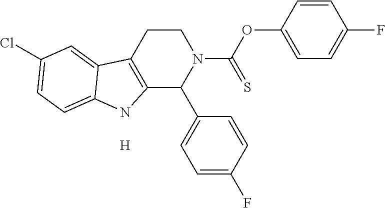 | 855 |
| 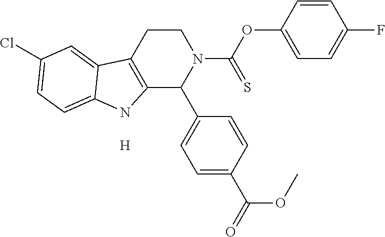 | 856 |
| 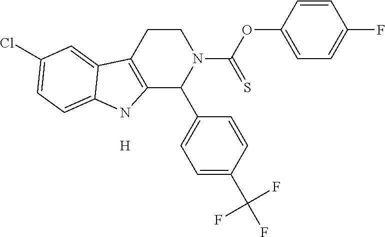 | 857 |
| 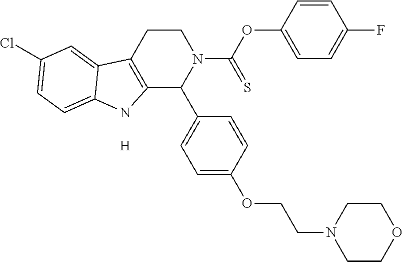 | 858 |
| 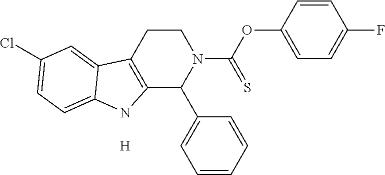 | 859 |
| 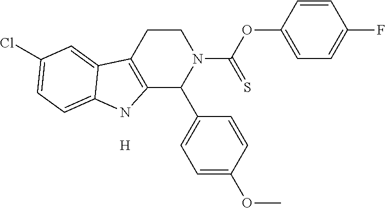 | 860 |
| 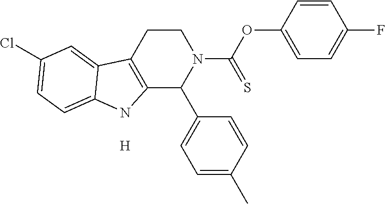 | 861 |
| 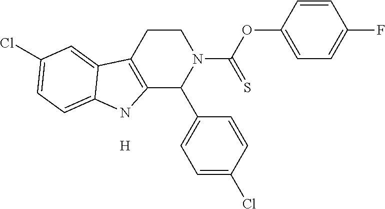 | 862 |
| 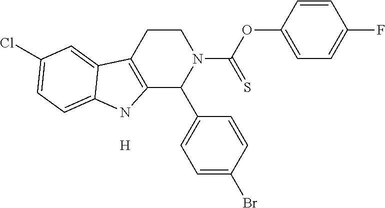 | 863 |
| 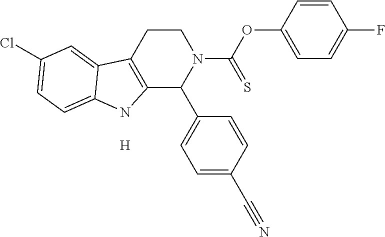 | 864 |
| 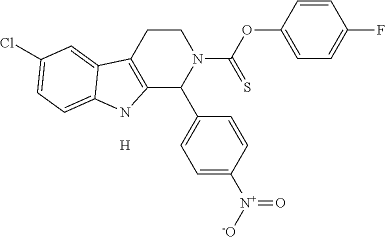 | 865 |
| 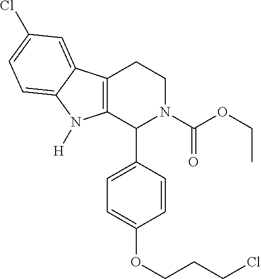 [SMILES: CCOC(=O)N1CCc2c([nH]c3ccc(Cl)cc23)C1c1ccc(OCCCCl)cc1] | 866 |
| 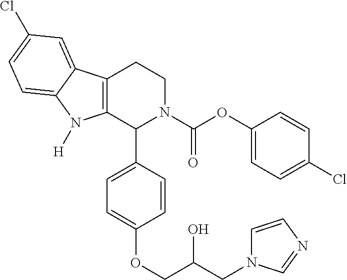 [SMILES: O=C(Oc1ccc(Cl)cc1)N1CCc2c([nH]c3ccc(Cl)cc23)C1c1ccc(OCC(O)Cn2ccnc2)cc1] | 867 |
| 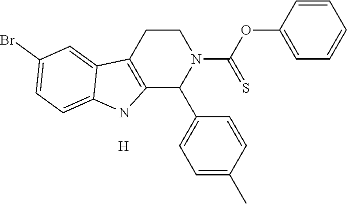 | 868 |
| 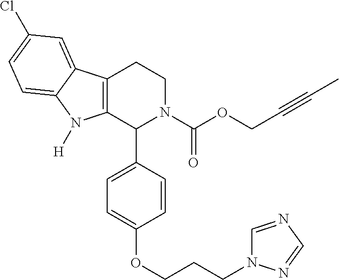 [SMILES: CC#CCOC(=O)N1CCc2c([nH]c3ccc(Cl)cc23)C1c1ccc(OCCCn2cncn2)cc1] | 869 |
| 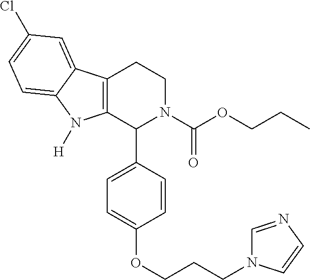 [SMILES: CCCOC(=O)N1CCc2c([nH]c3ccc(Cl)cc23)C1c1ccc(OCCCn2ccnc2)cc1] | 870 |
| 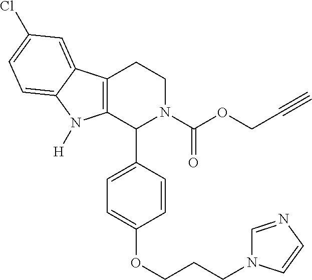 [SMILES: C#CCOC(=O)N1CCc2c([nH]c3ccc(Cl)cc23)C1c1ccc(OCCCn2ccnc2)cc1] | 871 |
| 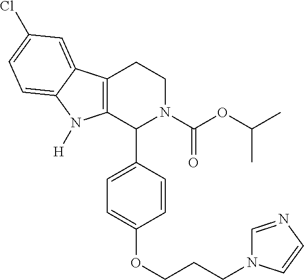 [SMILES: CC(C)OC(=O)N1CCc2c([nH]c3ccc(Cl)cc23)C1c1ccc(OCCCn2ccnc2)cc1] | 872 |
| 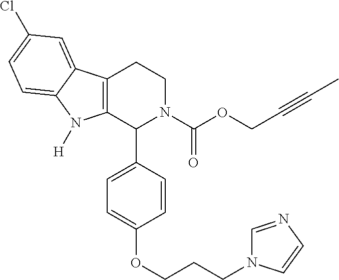 [SMILES: CC#CCOC(=O)N1CCc2c([nH]c3ccc(Cl)cc23)C1c1ccc(OCCCn2ccnc2)cc1] | 873 |
| 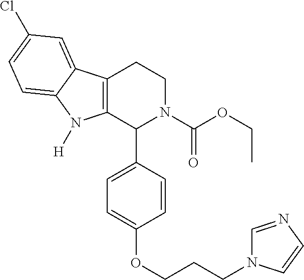 [SMILES: CCOC(=O)N1CCc2c([nH]c3ccc(Cl)cc23)C1c1ccc(OCCCn2ccnc2)cc1] | 874 |
| 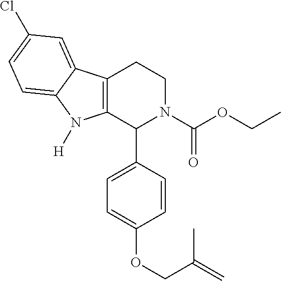 [SMILES: C=C(C)COc1ccc(C2c3[nH]c4ccc(Cl)cc4c3CCN2C(=O)OCC)cc1] | 875 |
| 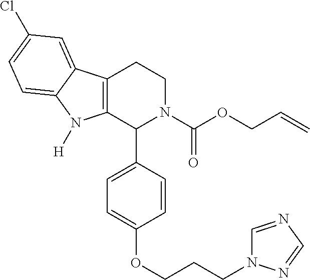 [SMILES: C=CCOC(=O)N1CCc2c([nH]c3ccc(Cl)cc23)C1c1ccc(OCCCn2cncn2)cc1] | 876 |
| 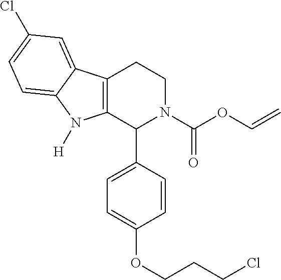 [SMILES: C=COC(=O)N1CCc2c([nH]c3ccc(Cl)cc23)C1c1ccc(OCCCCl)cc1] | 877 |
| 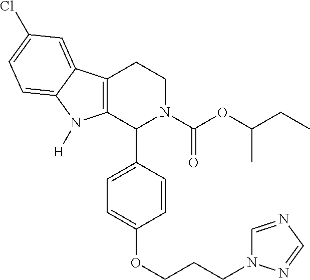 [SMILES: CCC(C)OC(=O)N1CCc2c([nH]c3ccc(Cl)cc23)C1c1ccc(OCCCn2cncn2)cc1] | 878 |
| 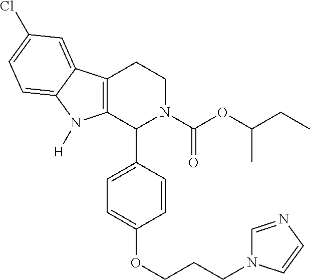 [SMILES: CCC(C)OC(=O)N1CCc2c([nH]c3ccc(Cl)cc23)C1c1ccc(OCCCn2ccnc2)cc1] | 879 |
| 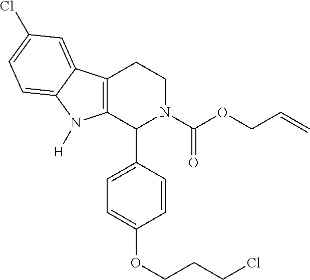 [SMILES: C=CCOC(=O)N1CCc2c([nH]c3ccc(Cl)cc23)C1c1ccc(OCCCCl)cc1] | 880 |
| 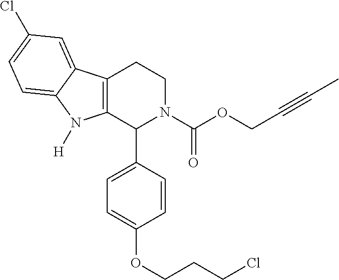 [SMILES: CC#CCOC(=O)N1CCc2c([nH]c3ccc(Cl)cc23)C1c1ccc(OCCCCl)cc1] | 881 |
| 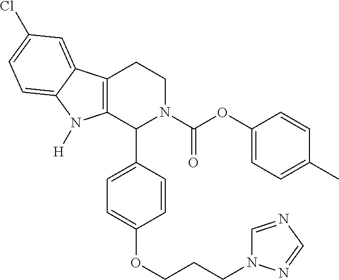 [SMILES: Cc1ccc(OC(=O)N2CCc3c([nH]c4ccc(Cl)cc34)C2c2ccc(OCCCn3cncn3)cc2)cc1] | 882 |
| 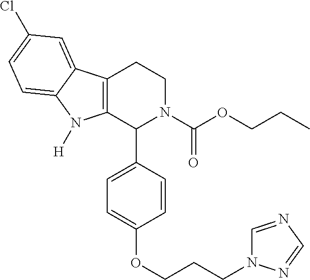 [SMILES: CCCOC(=O)N1CCc2c([nH]c3ccc(Cl)cc23)C1c1ccc(OCCCn2cncn2)cc1] | 883 |
| 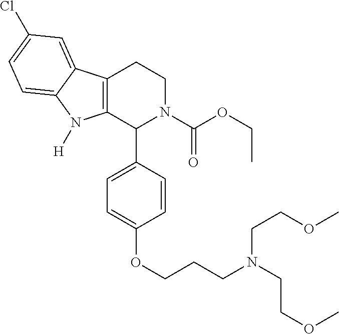 [SMILES: CCOC(=O)N1CCc2c([nH]c3ccc(Cl)cc23)C1c1ccc(OCCCN(CCOC)CCOC)cc1] | 884 |
| 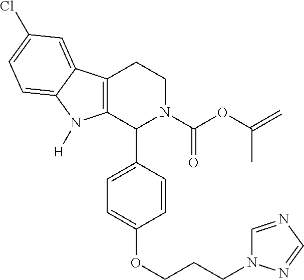 [SMILES: C=C(C)OC(=O)N1CCc2c([nH]c3ccc(Cl)cc23)C1c1ccc(OCCCn2cncn2)cc1] | 885 |
| 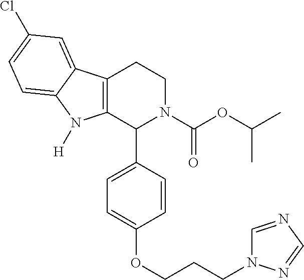 [SMILES: CC(C)OC(=O)N1CCc2c([nH]c3ccc(Cl)cc23)C1c1ccc(OCCCn2cncn2)cc1] | 886 |
| 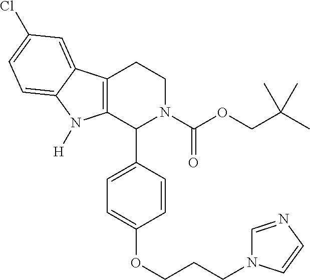 [SMILES: CC(C)(C)COC(=O)N1CCc2c([nH]c3ccc(Cl)cc23)C1c1ccc(OCCCn2ccnc2)cc1] | 887 |
| 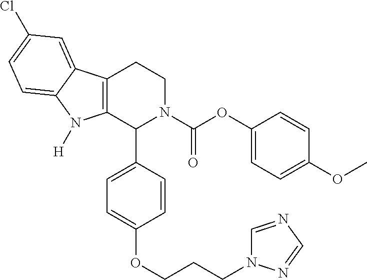 [SMILES: COc1ccc(OC(=O)N2CCc3c([nH]c4ccc(Cl)cc34)C2c2ccc(OCCCn3cncn3)cc2)cc1] | 888 |
| 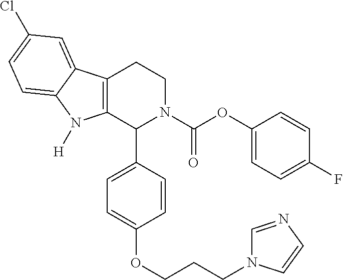 [SMILES: O=C(Oc1ccc(F)cc1)N1CCc2c([nH]c3ccc(Cl)cc23)C1c1ccc(OCCCn2ccnc2)cc1] | 889 |
| 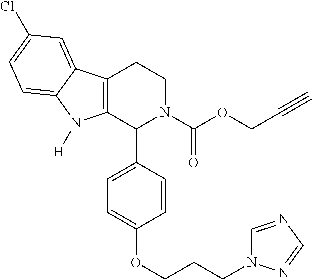 [SMILES: C#CCOC(=O)N1CCc2c([nH]c3ccc(Cl)cc23)C1c1ccc(OCCCn2cncn2)cc1] | 890 |
| 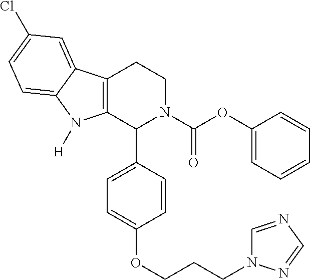 [SMILES: O=C(Oc1ccccc1)N1CCc2c([nH]c3ccc(Cl)cc23)C1c1ccc(OCCCn2cncn2)cc1] | 891 |
| 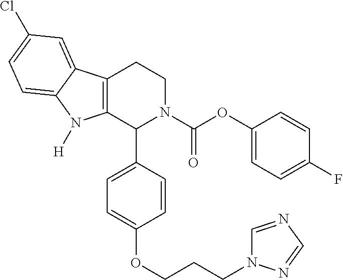 [SMILES: O=C(Oc1ccc(F)cc1)N1CCc2c([nH]c3ccc(Cl)cc23)C1c1ccc(OCCCn2cncn2)cc1] | 892 |
| 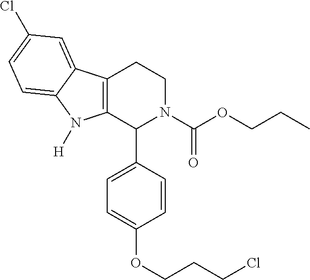 [SMILES: CCCOC(=O)N1CCc2c([nH]c3ccc(Cl)cc23)C1c1ccc(OCCCCl)cc1] | 893 |
| 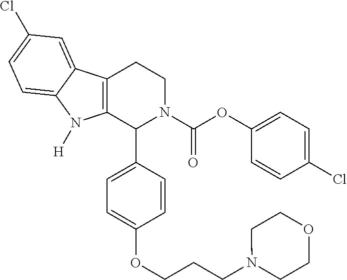 [SMILES: O=C(Oc1ccc(Cl)cc1)N1CCc2c([nH]c3ccc(Cl)cc23)C1c1ccc(OCCCN2CCOCC2)cc1] | 894 |
| 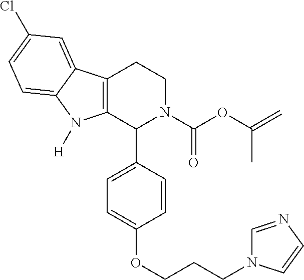 [SMILES: C=C(C)OC(=O)N1CCc2c([nH]c3ccc(Cl)cc23)C1c1ccc(OCCCn2ccnc2)cc1] | 895 |
| 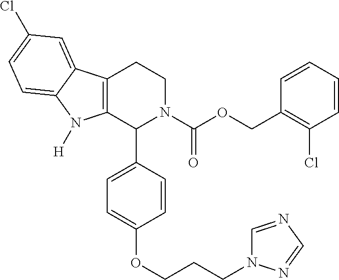 [SMILES: O=C(OCc1ccccc1Cl)N1CCc2c([nH]c3ccc(Cl)cc23)C1c1ccc(OCCCn2cncn2)cc1] | 896 |
| 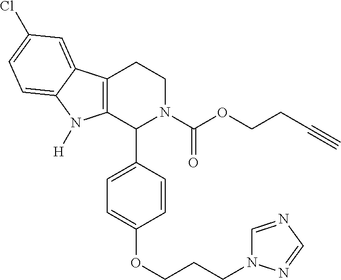 [SMILES: C#CCCOC(=O)N1CCc2c([nH]c3ccc(Cl)cc23)C1c1ccc(OCCCn2cncn2)cc1] | 897 |
| 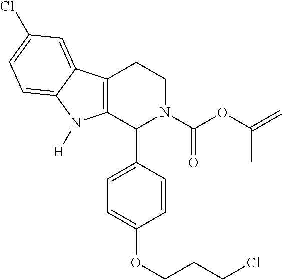 [SMILES: C=C(C)OC(=O)N1CCc2c([nH]c3ccc(Cl)cc23)C1c1ccc(OCCCCl)cc1] | 898 |
| 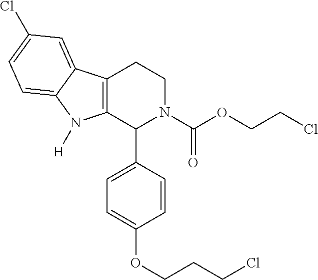 [SMILES: O=C(OCCCl)N1CCc2c([nH]c3ccc(Cl)cc23)C1c1ccc(OCCCCl)cc1] | 899 |
| 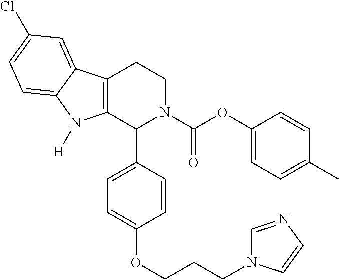 [SMILES: Cc1ccc(OC(=O)N2CCc3c([nH]c4ccc(Cl)cc34)C2c2ccc(OCCCn3ccnc3)cc2)cc1] | 900 |
| 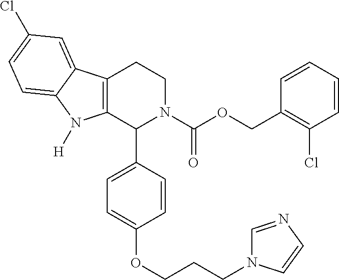 [SMILES: O=C(OCc1ccccc1Cl)N1CCc2c([nH]c3ccc(Cl)cc23)C1c1ccc(OCCCn2ccnc2)cc1] | 901 |
| 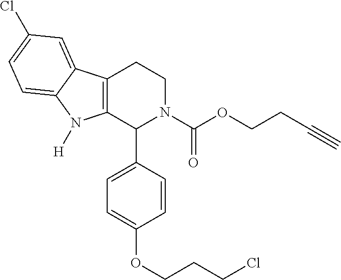 [SMILES: C#CCCOC(=O)N1CCc2c([nH]c3ccc(Cl)cc23)C1c1ccc(OCCCCl)cc1] | 902 |
| 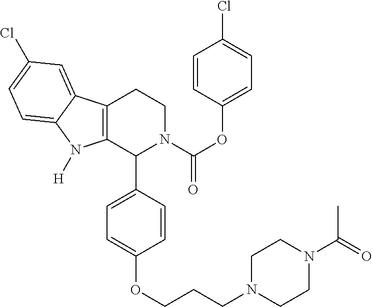 [SMILES: CC(=O)N1CCN(CCCOc2ccc(C3c4[nH]c5ccc(Cl)cc5c4CCN3C(=O)Oc3ccc(Cl)cc3)cc2)CC1] | 903 |
| 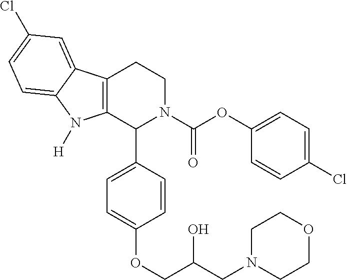 [SMILES: O=C(Oc1ccc(Cl)cc1)N1CCc2c([nH]c3ccc(Cl)cc23)C1c1ccc(OCC(O)CN2CCOCC2)cc1] | 904 |
| 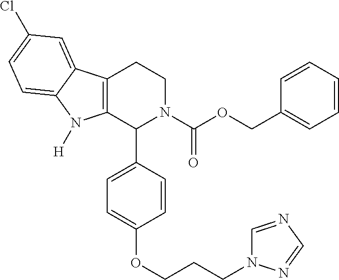 [SMILES: O=C(OCc1ccccc1)N1CCc2c([nH]c3ccc(Cl)cc23)C1c1ccc(OCCCn2cncn2)cc1] | 905 |
| 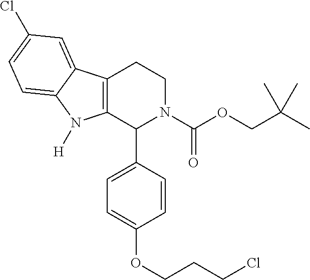 [SMILES: CC(C)(C)COC(=O)N1CCc2c([nH]c3ccc(Cl)cc23)C1c1ccc(OCCCCl)cc1] | 906 |
| 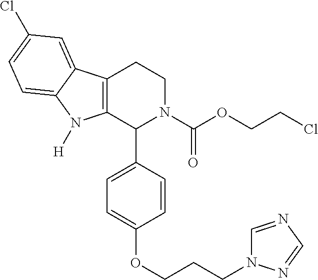 [SMILES: O=C(OCCCl)N1CCc2c([nH]c3ccc(Cl)cc23)C1c1ccc(OCCCn2cncn2)cc1] | 907 |
| 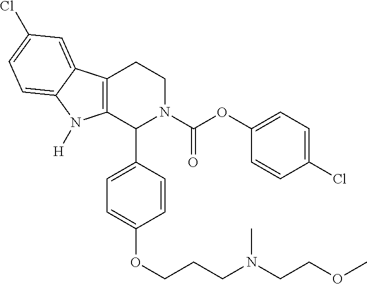 [SMILES: COCCN(C)CCCOc1ccc(C2c3[nH]c4ccc(Cl)cc4c3CCN2C(=O)Oc2ccc(Cl)cc2)cc1] | 908 |
| 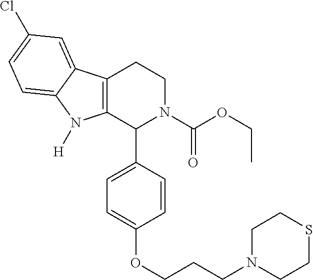 [SMILES: CCOC(=O)N1CCc2c([nH]c3ccc(Cl)cc23)C1c1ccc(OCCCN2CCSCC2)cc1] | 909 |
| 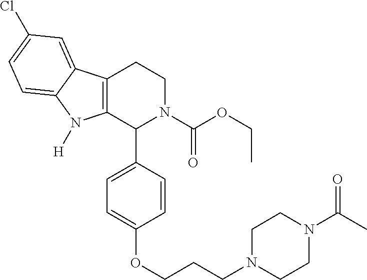 [SMILES: CCOC(=O)N1CCc2c([nH]c3ccc(Cl)cc23)C1c1ccc(OCCCN2CCN(C(C)=O)CC2)cc1] | 910 |
| 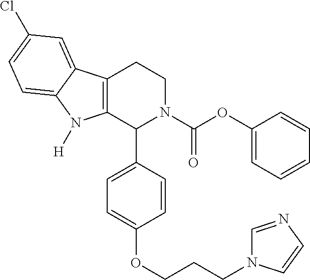 [SMILES: O=C(Oc1ccccc1)N1CCc2c([nH]c3ccc(Cl)cc23)C1c1ccc(OCCCn2ccnc2)cc1] | 911 |
| 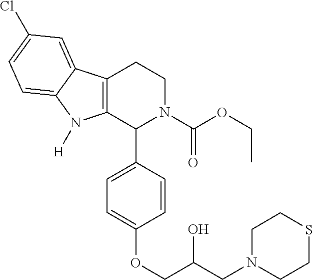 [SMILES: CCOC(=O)N1CCc2c([nH]c3ccc(Cl)cc23)C1c1ccc(OCC(O)CN2CCSCC2)cc1] | 912 |
| 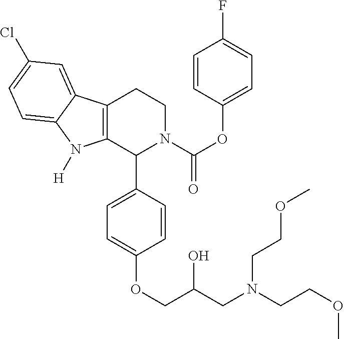 [SMILES: COCCN(CCOC)CC(O)COc1ccc(C2c3[nH]c4ccc(Cl)cc4c3CCN2C(=O)Oc2ccc(F)cc2)cc1] | 913 |
| 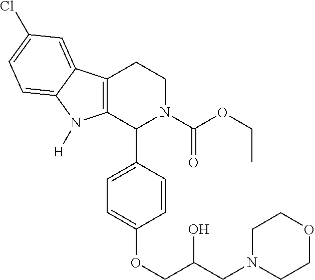 [SMILES: CCOC(=O)N1CCc2c([nH]c3ccc(Cl)cc23)C1c1ccc(OCC(O)CN2CCOCC2)cc1] | 914 |
| 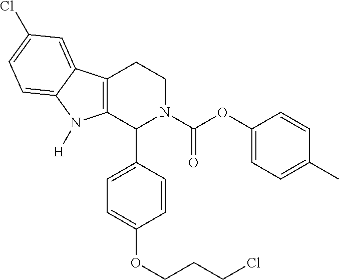 [SMILES: Cc1ccc(OC(=O)N2CCc3c([nH]c4ccc(Cl)cc34)C2c2ccc(OCCCCl)cc2)cc1] | 915 |
| 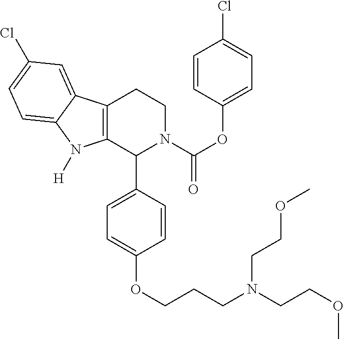 [SMILES: COCCN(CCCOc1ccc(C2c3[nH]c4ccc(Cl)cc4c3CCN2C(=O)Oc2ccc(Cl)cc2)cc1)CCOC] | 916 |
| 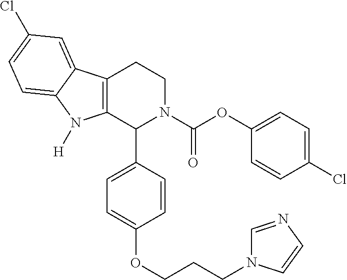 [SMILES: O=C(Oc1ccc(Cl)cc1)N1CCc2c([nH]c3ccc(Cl)cc23)C1c1ccc(OCCCn2ccnc2)cc1] | 917 |
| 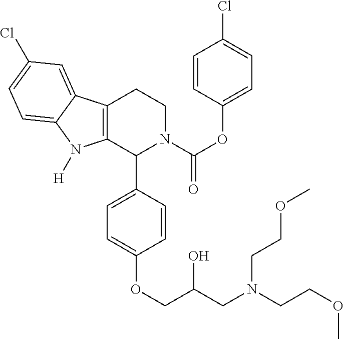 [SMILES: COCCN(CCOC)CC(O)COc1ccc(C2c3[nH]c4ccc(Cl)cc4c3CCN2C(=O)Oc2ccc(Cl)cc2)cc1] | 918 |
| 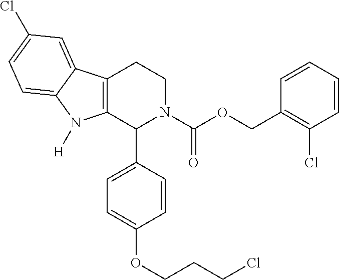 [SMILES: O=C(OCc1ccccc1Cl)N1CCc2c([nH]c3ccc(Cl)cc23)C1c1ccc(OCCCCl)cc1] | 919 |
| 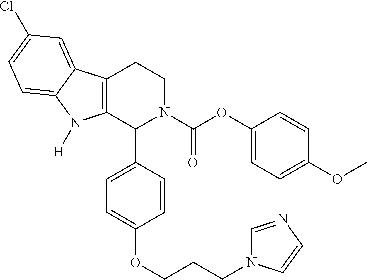 [SMILES: COc1ccc(OC(=O)N2CCc3c([nH]c4ccc(Cl)cc34)C2c2ccc(OCCCn3ccnc3)cc2)cc1] | 920 |
| 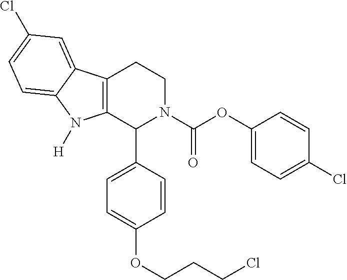 [SMILES: O=C(Oc1ccc(Cl)cc1)N1CCc2c([nH]c3ccc(Cl)cc23)C1c1ccc(OCCCCl)cc1] | 921 |
| 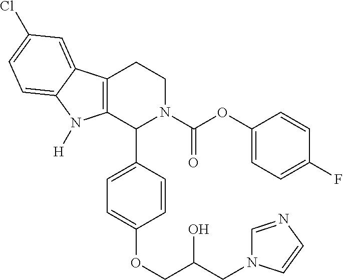 [SMILES: O=C(Oc1ccc(F)cc1)N1CCc2c([nH]c3ccc(Cl)cc23)C1c1ccc(OCC(O)Cn2ccnc2)cc1] | 922 |
| 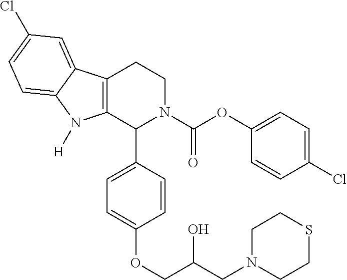 [SMILES: O=C(Oc1ccc(Cl)cc1)N1CCc2c([nH]c3ccc(Cl)cc23)C1c1ccc(OCC(O)CN2CCSCC2)cc1] | 923 |
| 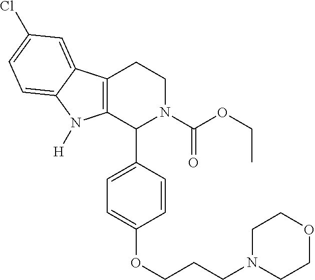 [SMILES: CCOC(=O)N1CCc2c([nH]c3ccc(Cl)cc23)C1c1ccc(OCCCN2CCOCC2)cc1] | 924 |
| 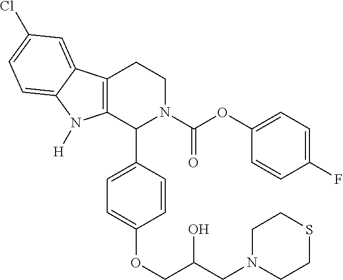 [SMILES: O=C(Oc1ccc(F)cc1)N1CCc2c([nH]c3ccc(Cl)cc23)C1c1ccc(OCC(O)CN2CCSCC2)cc1] | 925 |
| 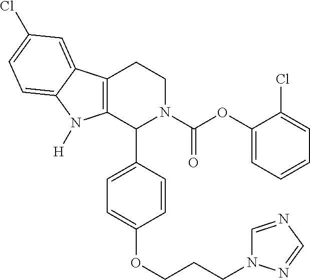 [SMILES: O=C(Oc1ccccc1Cl)N1CCc2c([nH]c3ccc(Cl)cc23)C1c1ccc(OCCCn2cncn2)cc1] | 926 |
| 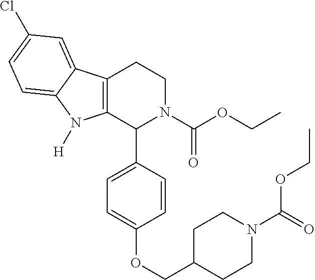 [SMILES: CCOC(=O)N1CCC(COc2ccc(C3c4[nH]c5ccc(Cl)cc5c4CCN3C(=O)OCC)cc2)CC1] | 927 |
| 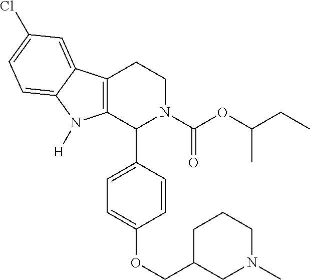 [SMILES: CCC(C)OC(=O)N1CCc2c([nH]c3ccc(Cl)cc23)C1c1ccc(OCC2CCCN(C)C2)cc1] | 928 |
| 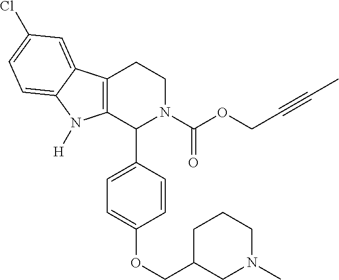 [SMILES: CC#CCOC(=O)N1CCc2c([nH]c3ccc(Cl)cc23)C1c1ccc(OCC2CCCN(C)C2)cc1] | 929 |
| 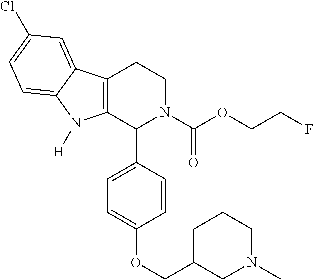 [SMILES: CN1CCCC(COc2ccc(C3c4[nH]c5ccc(Cl)cc5c4CCN3C(=O)OCCF)cc2)C1] | 930 |
| 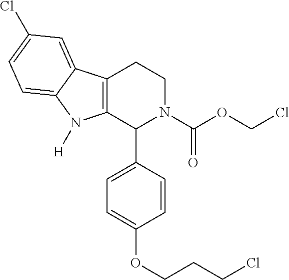 [SMILES: O=C(OCCl)N1CCc2c([nH]c3ccc(Cl)cc23)C1c1ccc(OCCCCl)cc1] | 931 |
| 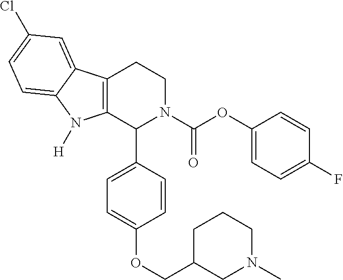 [SMILES: CN1CCCC(COc2ccc(C3c4[nH]c5ccc(Cl)cc5c4CCN3C(=O)Oc3ccc(F)cc3)cc2)C1] | 932 |
| 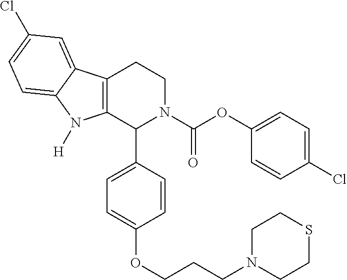 [SMILES: O=C(Oc1ccc(Cl)cc1)N1CCc2c([nH]c3ccc(Cl)cc23)C1c1ccc(OCCCN2CCSCC2)cc1] | 933 |
| 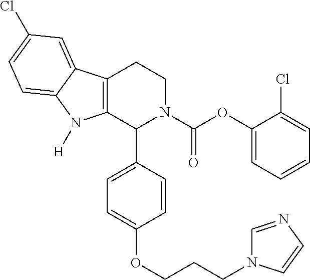 [SMILES: O=C(Oc1ccccc1Cl)N1CCc2c([nH]c3ccc(Cl)cc23)C1c1ccc(OCCCn2ccnc2)cc1] | 934 |
| 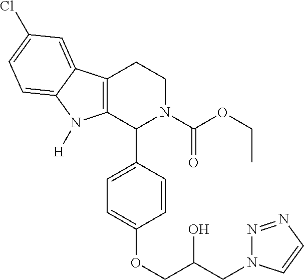 [SMILES: CCOC(=O)N1CCc2c([nH]c3ccc(Cl)cc23)C1c1ccc(OCC(O)Cn2ccnn2)cc1] | 935 |
| 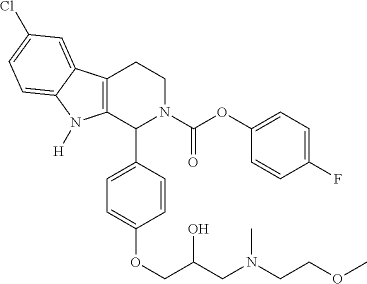 [SMILES: COCCN(C)CC(O)COc1ccc(C2c3[nH]c4ccc(Cl)cc4c3CCN2C(=O)Oc2ccc(F)cc2)cc1] | 936 |
| 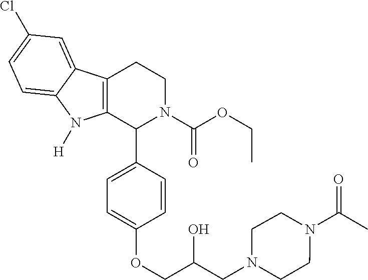 [SMILES: CCOC(=O)N1CCc2c([nH]c3ccc(Cl)cc23)C1c1ccc(OCC(O)CN2CCN(C(C)=O)CC2)cc1] | 937 |
| 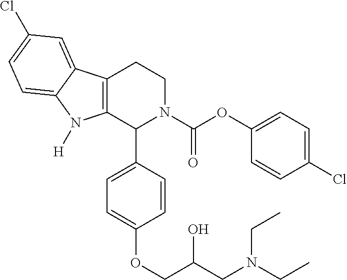 [SMILES: CCN(CC)CC(O)COc1ccc(C2c3[nH]c4ccc(Cl)cc4c3CCN2C(=O)Oc2ccc(Cl)cc2)cc1] | 938 |
| 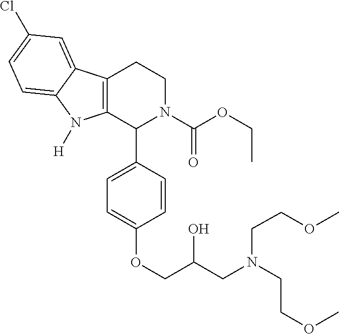 [SMILES: CCOC(=O)N1CCc2c([nH]c3ccc(Cl)cc23)C1c1ccc(OCC(O)CN(CCOC)CCOC)cc1] | 939 |
| 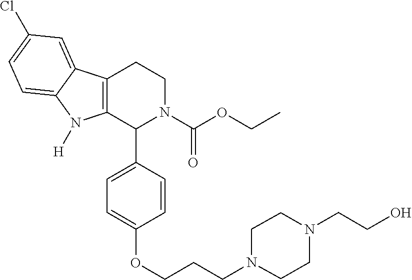 [SMILES: CCOC(=O)N1CCc2c([nH]c3ccc(Cl)cc23)C1c1ccc(OCCCN2CCN(CCO)CC2)cc1] | 940 |
| 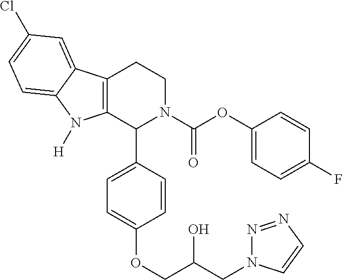 [SMILES: O=C(Oc1ccc(F)cc1)N1CCc2c([nH]c3ccc(Cl)cc23)C1c1ccc(OCC(O)Cn2ccnn2)cc1] | 941 |
| 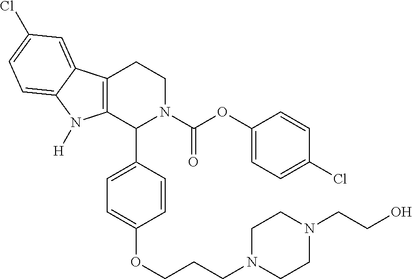 [SMILES: O=C(Oc1ccc(Cl)cc1)N1CCc2c([nH]c3ccc(Cl)cc23)C1c1ccc(OCCCN2CCN(CCO)CC2)cc1] | 942 |
| 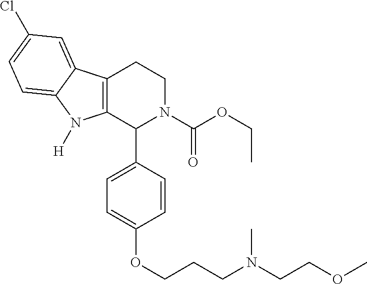 [SMILES: CCOC(=O)N1CCc2c([nH]c3ccc(Cl)cc23)C1c1ccc(OCCCN(C)CCOC)cc1] | 943 |
| 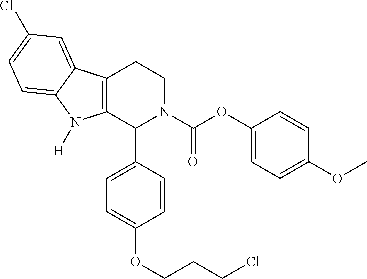 [SMILES: COc1ccc(OC(=O)N2CCc3c([nH]c4ccc(Cl)cc34)C2c2ccc(OCCCCl)cc2)cc1] | 944 |
| 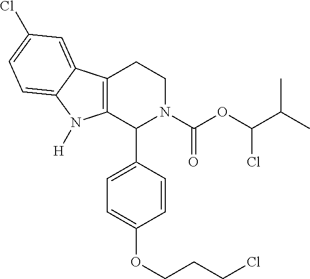 [SMILES: CC(C)C(Cl)OC(=O)N1CCc2c([nH]c3ccc(Cl)cc23)C1c1ccc(OCCCCl)cc1] | 945 |
| 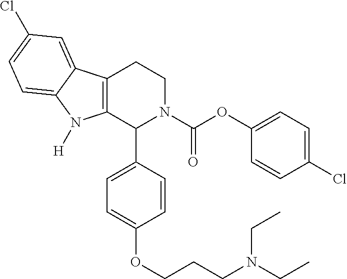 [SMILES: CCN(CC)CCCOc1ccc(C2c3[nH]c4ccc(Cl)cc4c3CCN2C(=O)Oc2ccc(Cl)cc2)cc1] | 946 |
| 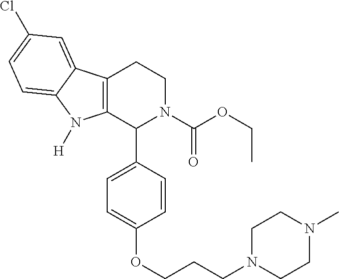 [SMILES: CCOC(=O)N1CCc2c([nH]c3ccc(Cl)cc23)C1c1ccc(OCCCN2CCN(C)CC2)cc1] | 947 |
| 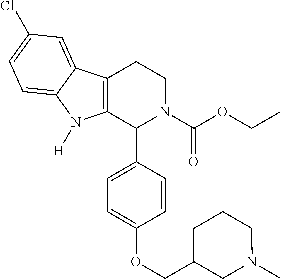 [SMILES: CCOC(=O)N1CCc2c([nH]c3ccc(Cl)cc23)C1c1ccc(OCC2CCCN(C)C2)cc1] | 948 |
| 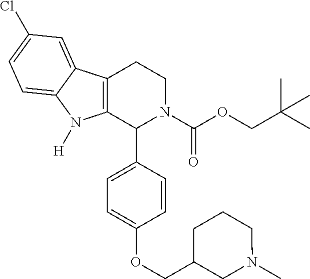 [SMILES: CN1CCCC(COc2ccc(C3c4[nH]c5ccc(Cl)cc5c4CCN3C(=O)OCC(C)(C)C)cc2)C1] | 949 |
| 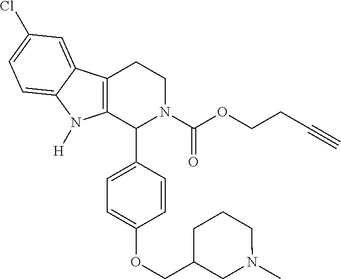 [SMILES: C#CCCOC(=O)N1CCc2c([nH]c3ccc(Cl)cc23)C1c1ccc(OCC2CCCN(C)C2)cc1] | 950 |
| 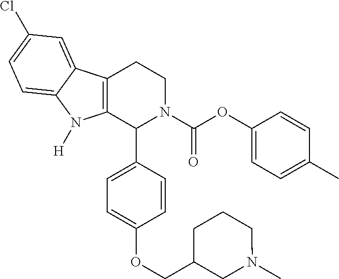 [SMILES: Cc1ccc(OC(=O)N2CCc3c([nH]c4ccc(Cl)cc34)C2c2ccc(OCC3CCCN(C)C3)cc2)cc1] | 951 |
| 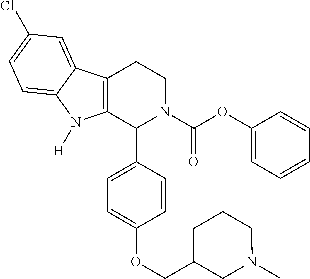 [SMILES: CN1CCCC(COc2ccc(C3c4[nH]c5ccc(Cl)cc5c4CCN3C(=O)Oc3ccccc3)cc2)C1] | 952 |
| 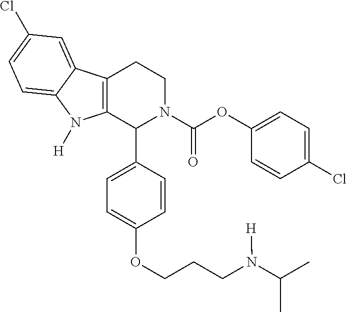 [SMILES: CC(C)NCCCOc1ccc(C2c3[nH]c4ccc(Cl)cc4c3CCN2C(=O)Oc2ccc(Cl)cc2)cc1] | 953 |
| 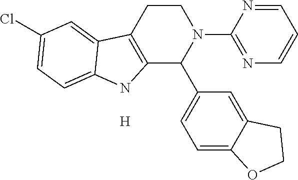 | 954 |
| 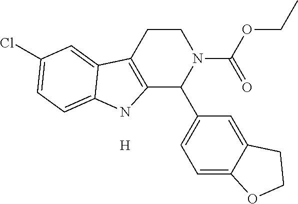 | 955 |
| 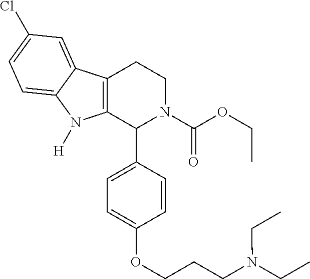 [SMILES: CCOC(=O)N1CCc2c([nH]c3ccc(Cl)cc23)C1c1ccc(OCCCN(CC)CC)cc1] | 956 |
| 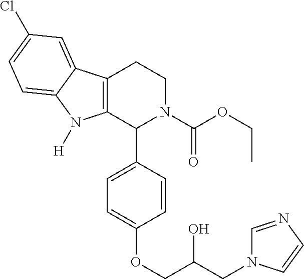 [SMILES: CCOC(=O)N1CCc2c([nH]c3ccc(Cl)cc23)C1c1ccc(OCC(O)Cn2ccnc2)cc1] | 957 |
| 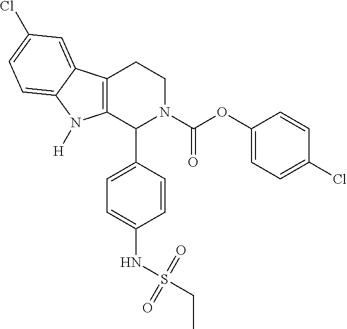 [SMILES: CCS(=O)(=O)Nc1ccc(C2c3[nH]c4ccc(Cl)cc4c3CCN2C(=O)Oc2ccc(Cl)cc2)cc1] | 958 |
| 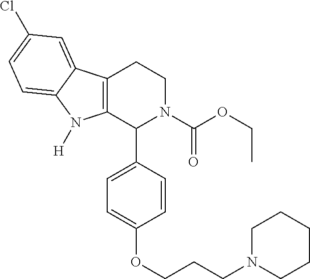 [SMILES: CCOC(=O)N1CCc2c([nH]c3ccc(Cl)cc23)C1c1ccc(OCCCN2CCCCC2)cc1] | 959 |
| 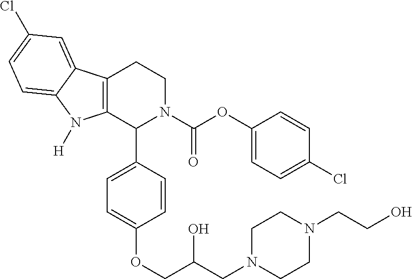 [SMILES: O=C(Oc1ccc(Cl)cc1)N1CCc2c([nH]c3ccc(Cl)cc23)C1c1ccc(OCC(O)CN2CCN(CCO)CC2)cc1] | 960 |
| 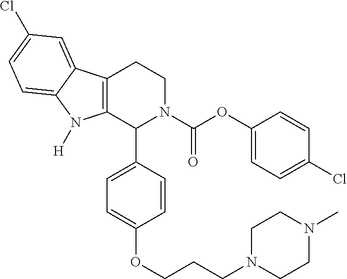 [SMILES: CN1CCN(CCCOc2ccc(C3c4[nH]c5ccc(Cl)cc5c4CCN3C(=O)Oc3ccc(Cl)cc3)cc2)CC1] | 961 |
| 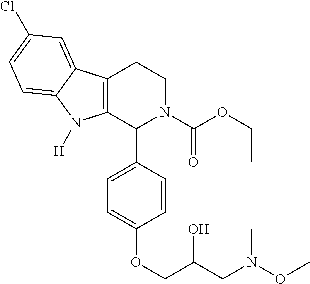 [SMILES: CCOC(=O)N1CCc2c([nH]c3ccc(Cl)cc23)C1c1ccc(OCC(O)CN(C)OC)cc1] | 962 |
| 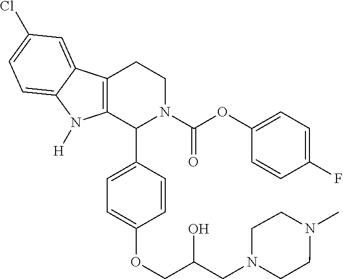 [SMILES: CN1CCN(CC(O)COc2ccc(C3c4[nH]c5ccc(Cl)cc5c4CCN3C(=O)Oc3ccc(F)cc3)cc2)CC1] | 963 |
| 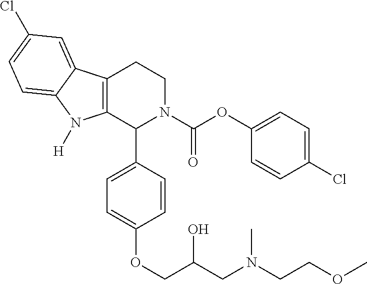 [SMILES: COCCN(C)CC(O)COc1ccc(C2c3[nH]c4ccc(Cl)cc4c3CCN2C(=O)Oc2ccc(Cl)cc2)cc1] | 964 |
| 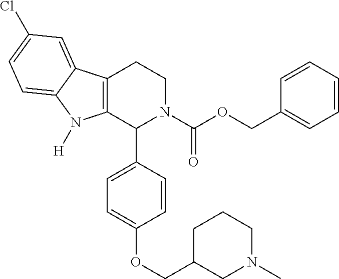 [SMILES: CN1CCCC(COc2ccc(C3c4[nH]c5ccc(Cl)cc5c4CCN3C(=O)OCc3ccccc3)cc2)C1] | 965 |
| 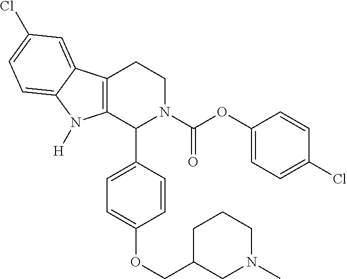 [SMILES: CN1CCCC(COc2ccc(C3c4[nH]c5ccc(Cl)cc5c4CCN3C(=O)Oc3ccc(Cl)cc3)cc2)C1] | 966 |
| 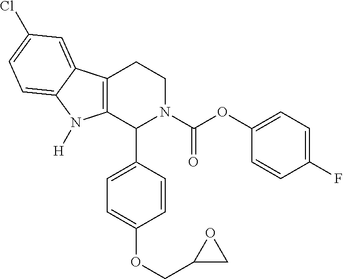 [SMILES: O=C(Oc1ccc(F)cc1)N1CCc2c([nH]c3ccc(Cl)cc23)C1c1ccc(OCC2CO2)cc1] | 967 |
| 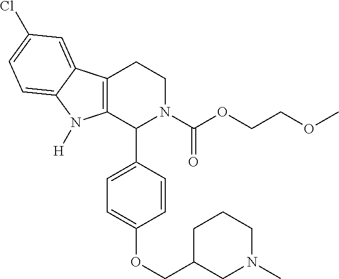 [SMILES: COCCOC(=O)N1CCc2c([nH]c3ccc(Cl)cc23)C1c1ccc(OCC2CCCN(C)C2)cc1] | 968 |
| 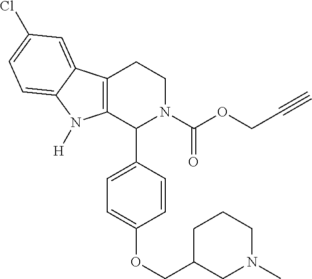 [SMILES: C#CCOC(=O)N1CCc2c([nH]c3ccc(Cl)cc23)C1c1ccc(OCC2CCCN(C)C2)cc1] | 969 |
| 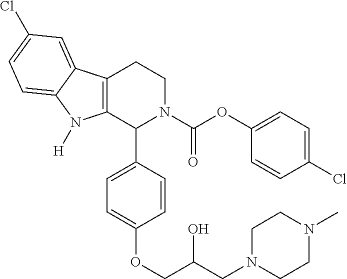 [SMILES: CN1CCN(CC(O)COc2ccc(C3c4[nH]c5ccc(Cl)cc5c4CCN3C(=O)Oc3ccc(Cl)cc3)cc2)CC1] | 970 |
| 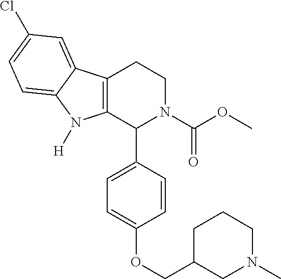 [SMILES: COC(=O)N1CCc2c([nH]c3ccc(Cl)cc23)C1c1ccc(OCC2CCCN(C)C2)cc1] | 971 |
| 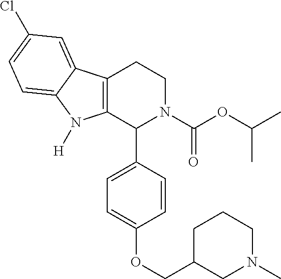 [SMILES: CC(C)OC(=O)N1CCc2c([nH]c3ccc(Cl)cc23)C1c1ccc(OCC2CCCN(C)C2)cc1] | 972 |
| 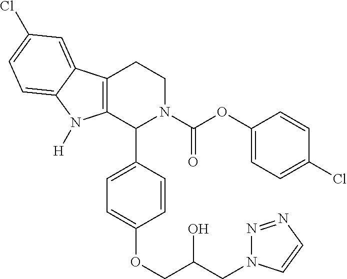 [SMILES: O=C(Oc1ccc(Cl)cc1)N1CCc2c([nH]c3ccc(Cl)cc23)C1c1ccc(OCC(O)Cn2ccnn2)cc1] | 973 |
| 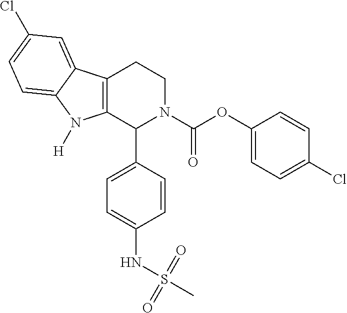 [SMILES: CS(=O)(=O)Nc1ccc(C2c3[nH]c4ccc(Cl)cc4c3CCN2C(=O)Oc2ccc(Cl)cc2)cc1] | 974 |
| 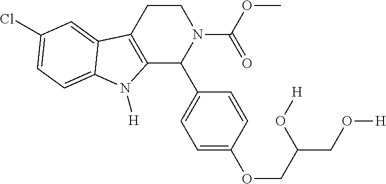 [SMILES: COC(=O)N1CCc2c([nH]c3ccc(Cl)cc23)C1c1ccc(OCC(O)CO)cc1] | 975 |
| 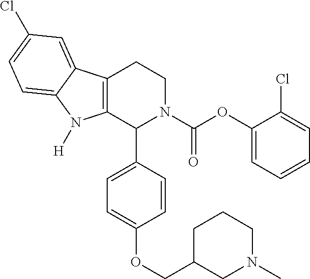 [SMILES: CN1CCCC(COc2ccc(C3c4[nH]c5ccc(Cl)cc5c4CCN3C(=O)Oc3ccccc3Cl)cc2)C1] | 976 |
| 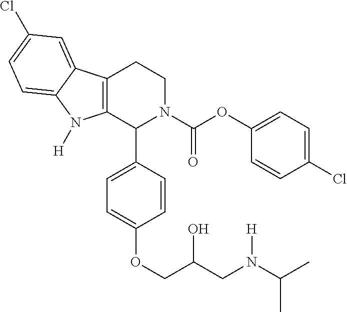 [SMILES: CC(C)NCC(O)COc1ccc(C2c3[nH]c4ccc(Cl)cc4c3CCN2C(=O)Oc2ccc(Cl)cc2)cc1] | 977 |
| 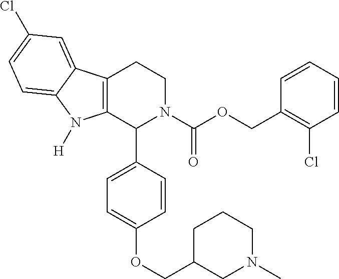 [SMILES: CN1CCCC(COc2ccc(C3c4[nH]c5ccc(Cl)cc5c4CCN3C(=O)OCc3ccccc3Cl)cc2)C1] | 978 |
| 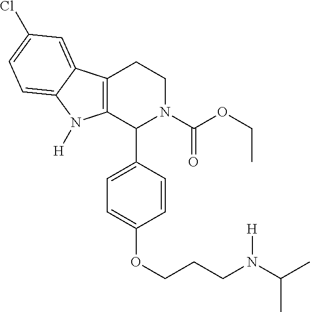 [SMILES: CCOC(=O)N1CCc2c([nH]c3ccc(Cl)cc23)C1c1ccc(OCCCNC(C)C)cc1] | 979 |
| 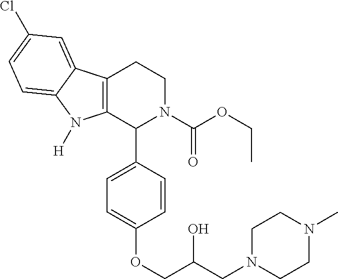 [SMILES: CCOC(=O)N1CCc2c([nH]c3ccc(Cl)cc23)C1c1ccc(OCC(O)CN2CCN(C)CC2)cc1] | 980 |
| 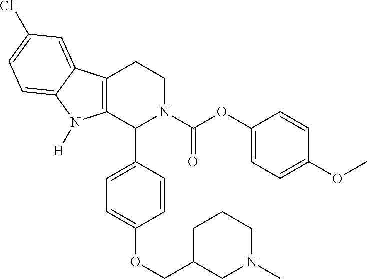 [SMILES: COc1ccc(OC(=O)N2CCc3c([nH]c4ccc(Cl)cc34)C2c2ccc(OCC3CCCN(C)C3)cc2)cc1] | 981 |
| 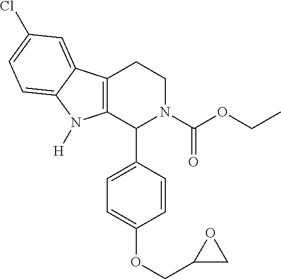 [SMILES: CCOC(=O)N1CCc2c([nH]c3ccc(Cl)cc23)C1c1ccc(OCC2CO2)cc1] | 982 |
| 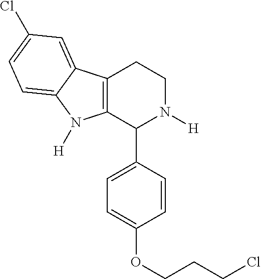 [SMILES: ClCCCOc1ccc(C2NCCc3c2[nH]c2ccc(Cl)cc32)cc1] | 983 |
| 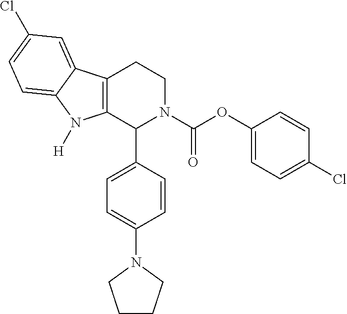 [SMILES: O=C(Oc1ccc(Cl)cc1)N1CCc2c([nH]c3ccc(Cl)cc23)C1c1ccc(N2CCCC2)cc1] | 984 |
| 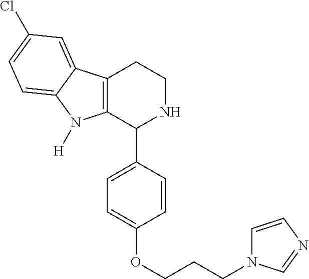 [SMILES: Clc1ccc2[nH]c3c(c2c1)CCNC3c1ccc(OCCCn2ccnc2)cc1] | 985 |
| 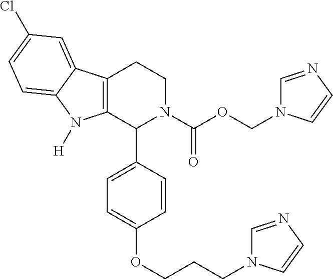 [SMILES: O=C(OCn1ccnc1)N1CCc2c([nH]c3ccc(Cl)cc23)C1c1ccc(OCCCn2ccnc2)cc1] | 986 |
| 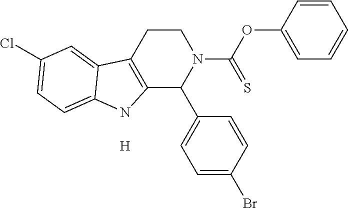 | 987 |
| 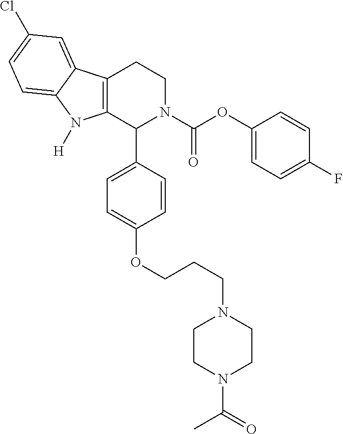 [SMILES: CC(=O)N1CCN(CCCOc2ccc(C3c4[nH]c5ccc(Cl)cc5c4CCN3C(=O)Oc3ccc(F)cc3)cc2)CC1] | 988 |
| 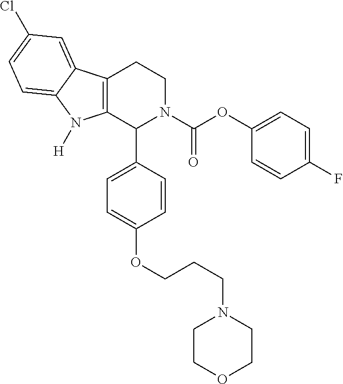 [SMILES: O=C(Oc1ccc(F)cc1)N1CCc2c([nH]c3ccc(Cl)cc23)C1c1ccc(OCCCN2CCOCC2)cc1] | 989 |
| 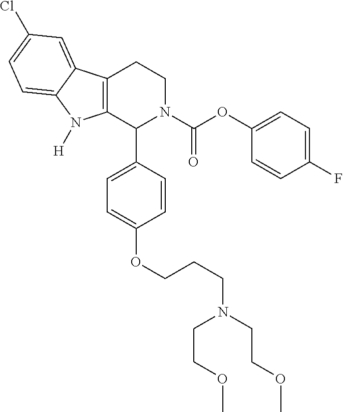 [SMILES: COCCN(CCCOc1ccc(C2c3[nH]c4ccc(Cl)cc4c3CCN2C(=O)Oc2ccc(F)cc2)cc1)CCOC] | 990 |
| 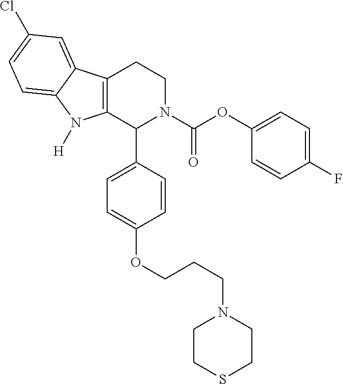 [SMILES: O=C(Oc1ccc(F)cc1)N1CCc2c([nH]c3ccc(Cl)cc23)C1c1ccc(OCCCN2CCSCC2)cc1] | 991 |
| 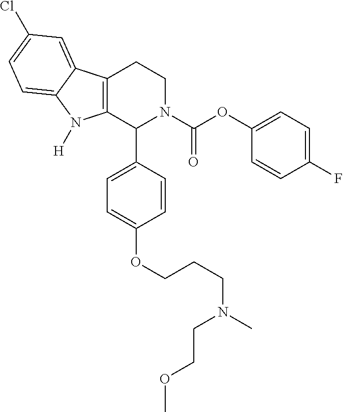 [SMILES: COCCN(C)CCCOc1ccc(C2c3[nH]c4ccc(Cl)cc4c3CCN2C(=O)Oc2ccc(F)cc2)cc1] | 992 |
| 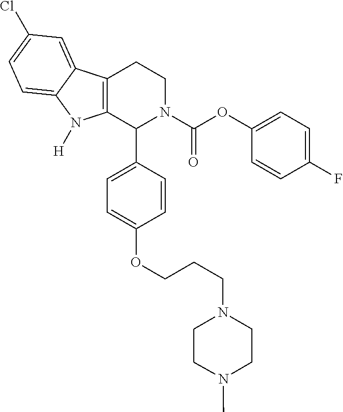 [SMILES: CN1CCN(CCCOc2ccc(C3c4[nH]c5ccc(Cl)cc5c4CCN3C(=O)Oc3ccc(F)cc3)cc2)CC1] | 993 |
| 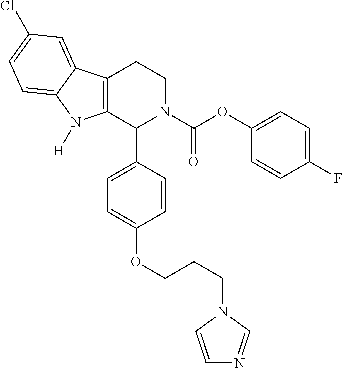 [SMILES: O=C(Oc1ccc(F)cc1)N1CCc2c([nH]c3ccc(Cl)cc23)C1c1ccc(OCCCn2ccnc2)cc1] | 994 |
| 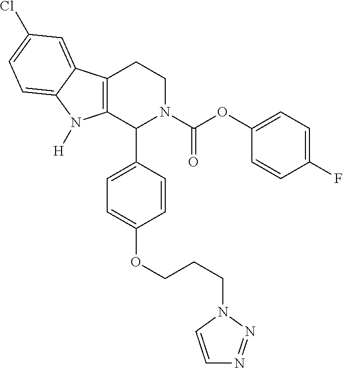 [SMILES: O=C(Oc1ccc(F)cc1)N1CCc2c([nH]c3ccc(Cl)cc23)C1c1ccc(OCCCn2ccnn2)cc1] | 995 |
| 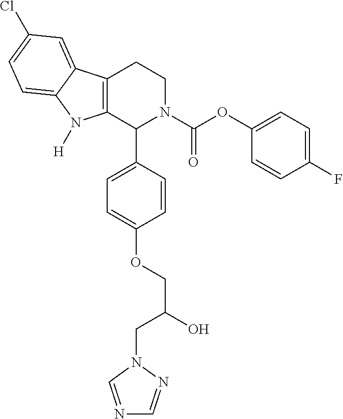 [SMILES: O=C(Oc1ccc(F)cc1)N1CCc2c([nH]c3ccc(Cl)cc23)C1c1ccc(OCC(O)Cn2cncn2)cc1] | 996 |
| 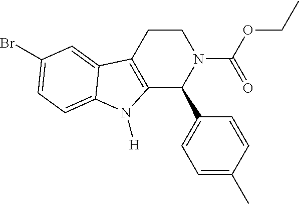 [SMILES: CCOC(=O)N1CCc2c([nH]c3ccc(Br)cc23)[C@@H]1c1ccc(C)cc1] | #8 |
| 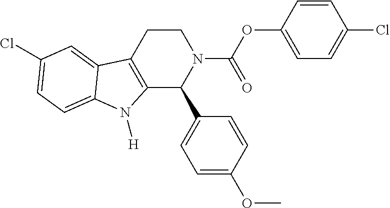 [SMILES: COc1ccc([C@H]2c3[nH]c4ccc(Cl)cc4c3CCN2C(=O)Oc2ccc(Cl)cc2)cc1] | #10 |
| 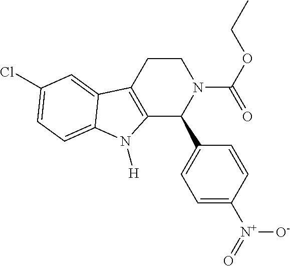 [SMILES: CCOC(=O)N1CCc2c([nH]c3ccc(Cl)cc23)[C@@H]1c1ccc([N+](=O)[O-])cc1] | #74 |
| 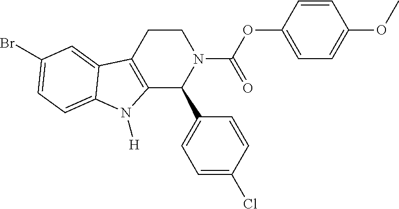 [SMILES: COc1ccc(OC(=O)N2CCc3c([nH]c4ccc(Br)cc34)[C@@H]2c2ccc(Cl)cc2)cc1] | #332 |
| 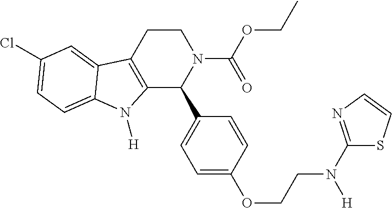 [SMILES: CCOC(=O)N1CCc2c([nH]c3ccc(Cl)cc23)[C@@H]1c1ccc(OCCNc2nccs2)cc1] | #816 |
| 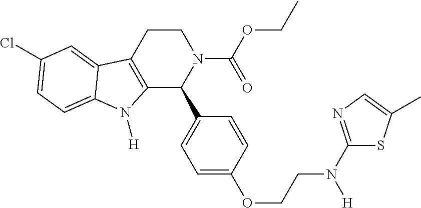 [SMILES: CCOC(=O)N1CCc2c([nH]c3ccc(Cl)cc23)[C@@H]1c1ccc(OCCNc2ncc(C)s2)cc1] | #817 |
| 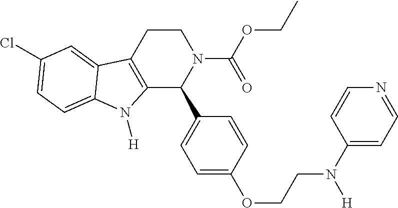 [SMILES: CCOC(=O)N1CCc2c([nH]c3ccc(Cl)cc23)[C@@H]1c1ccc(OCCNc2ccncc2)cc1] | #818 |
| 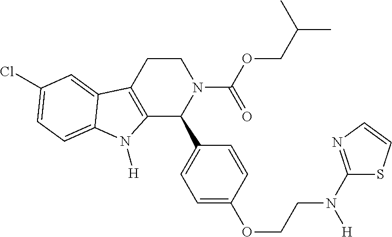 [SMILES: CC(C)COC(=O)N1CCc2c([nH]c3ccc(Cl)cc23)[C@@H]1c1ccc(OCCNc2nccs2)cc1] | #823 |
| 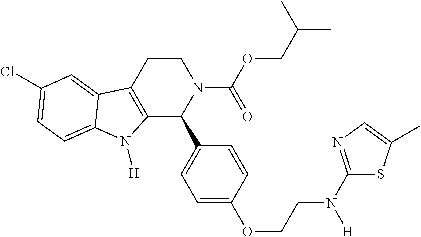 [SMILES: Cc1cnc(NCCOc2ccc([C@H]3c4[nH]c5ccc(Cl)cc5c4CCN3C(=O)OCC(C)C)cc2)s1] | #824 |
| 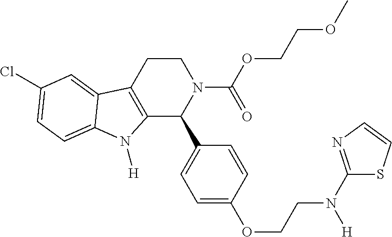 [SMILES: COCCOC(=O)N1CCc2c([nH]c3ccc(Cl)cc23)[C@@H]1c1ccc(OCCNc2nccs2)cc1] | #825 |
| 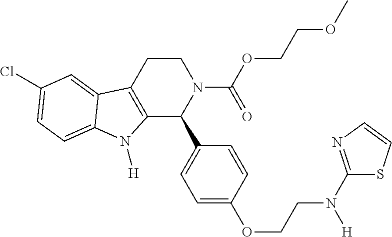 [SMILES: COCCOC(=O)N1CCc2c([nH]c3ccc(Cl)cc23)[C@@H]1c1ccc(OCCNc2nccs2)cc1] | #830 |
| 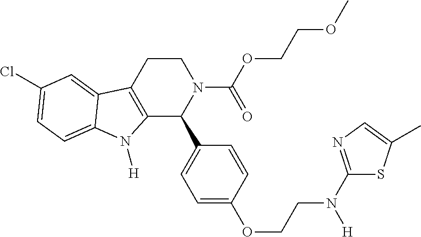 [SMILES: COCCOC(=O)N1CCc2c([nH]c3ccc(Cl)cc23)[C@@H]1c1ccc(OCCNc2ncc(C)s2)cc1] | #831 |
| 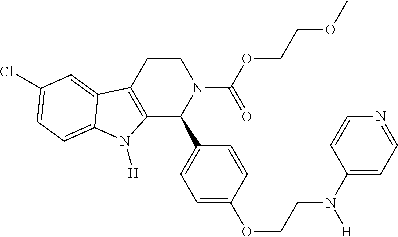 [SMILES: COCCOC(=O)N1CCc2c([nH]c3ccc(Cl)cc23)[C@@H]1c1ccc(OCCNc2ccncc2)cc1] | #832 |
| 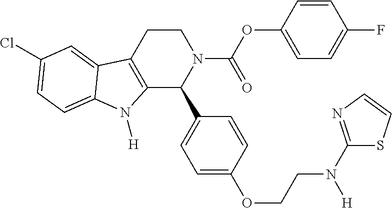 [SMILES: O=C(Oc1ccc(F)cc1)N1CCc2c([nH]c3ccc(Cl)cc23)[C@@H]1c1ccc(OCCNc2nccs2)cc1] | #837 |
| 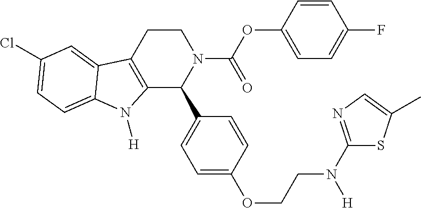 [SMILES: Cc1cnc(NCCOc2ccc([C@H]3c4[nH]c5ccc(Cl)cc5c4CCN3C(=O)Oc3ccc(F)cc3)cc2)s1] | #838 |
| 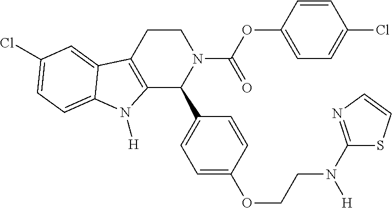 [SMILES: O=C(Oc1ccc(Cl)cc1)N1CCc2c([nH]c3ccc(Cl)cc23)[C@@H]1c1ccc(OCCNc2nccs2)cc1] | #841 |
| 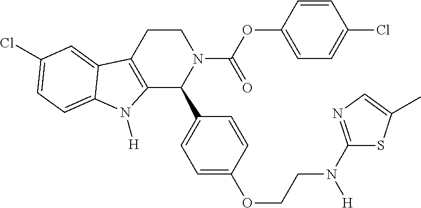 [SMILES: Cc1cnc(NCCOc2ccc([C@H]3c4[nH]c5ccc(Cl)cc5c4CCN3C(=O)Oc3ccc(Cl)cc3)cc2)s1] | #842 |
| 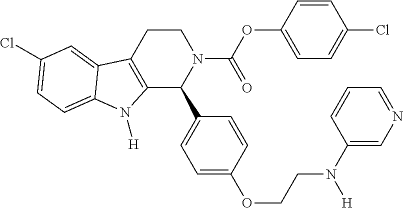 [SMILES: O=C(Oc1ccc(Cl)cc1)N1CCc2c([nH]c3ccc(Cl)cc23)[C@@H]1c1ccc(OCCNc2cccnc2)cc1] | #843 |
| 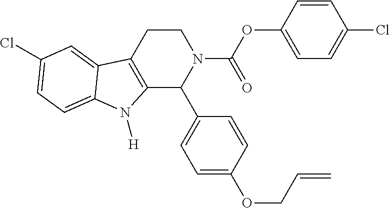 [SMILES: C=CCOc1ccc(C2c3[nH]c4ccc(Cl)cc4c3CCN2C(=O)Oc2ccc(Cl)cc2)cc1] | 999 |
| 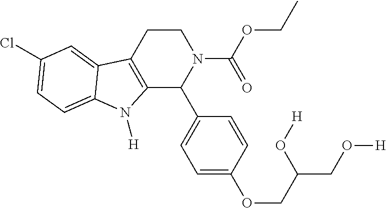 [SMILES: CCOC(=O)N1CCc2c([nH]c3ccc(Cl)cc23)C1c1ccc(OCC(O)CO)cc1] | 1000 |
| 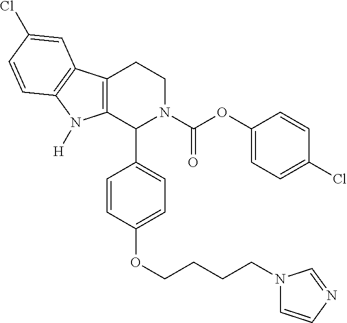 [SMILES: O=C(Oc1ccc(Cl)cc1)N1CCc2c([nH]c3ccc(Cl)cc23)C1c1ccc(OCCCCn2ccnc2)cc1] | 1001 |
| 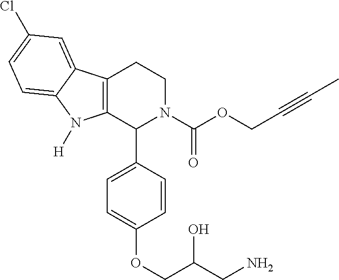 [SMILES: CC#CCOC(=O)N1CCc2c([nH]c3ccc(Cl)cc23)C1c1ccc(OCC(O)CN)cc1] | 1002 |
| 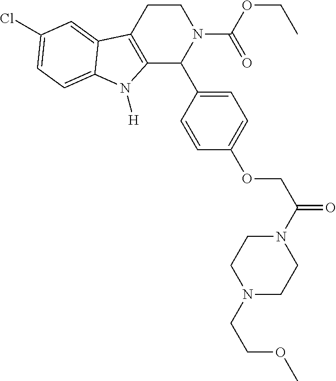 [SMILES: CCOC(=O)N1CCc2c([nH]c3ccc(Cl)cc23)C1c1ccc(OCC(=O)N2CCN(CCOC)CC2)cc1] | 1003 |
| 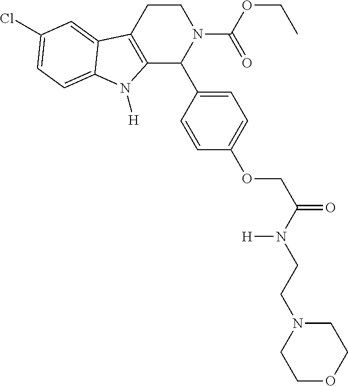 [SMILES: CCOC(=O)N1CCc2c([nH]c3ccc(Cl)cc23)C1c1ccc(OCC(=O)NCCN2CCOCC2)cc1] | 1004 |
| 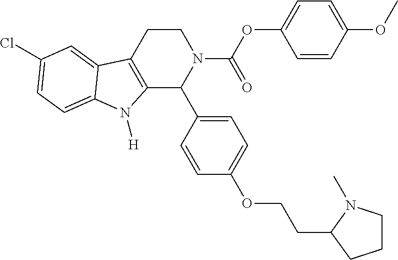 [SMILES: COc1ccc(OC(=O)N2CCc3c([nH]c4ccc(Cl)cc34)C2c2ccc(OCCC3CCCN3C)cc2)cc1] | 1005 |
| 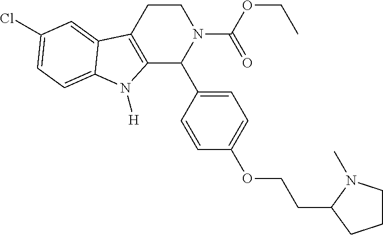 [SMILES: CCOC(=O)N1CCc2c([nH]c3ccc(Cl)cc23)C1c1ccc(OCCC2CCCN2C)cc1] | 1006 |
| 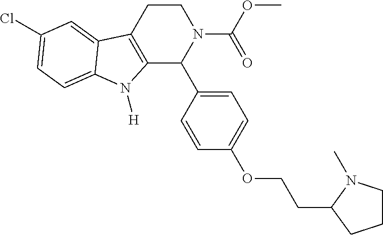 [SMILES: COC(=O)N1CCc2c([nH]c3ccc(Cl)cc23)C1c1ccc(OCCC2CCCN2C)cc1] | 1007 |
| 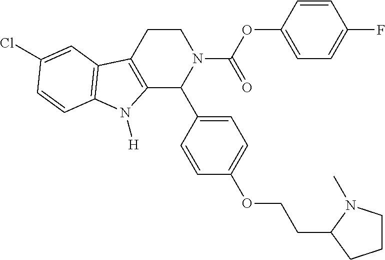 [SMILES: CN1CCCC1CCOc1ccc(C2c3[nH]c4ccc(Cl)cc4c3CCN2C(=O)Oc2ccc(F)cc2)cc1] | 1008 |
| 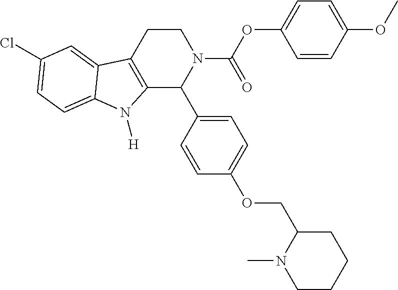 [SMILES: COc1ccc(OC(=O)N2CCc3c([nH]c4ccc(Cl)cc34)C2c2ccc(OCC3CCCCN3C)cc2)cc1] | 1009 |
| 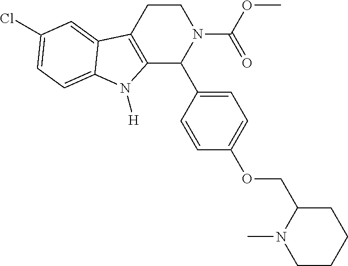 [SMILES: COC(=O)N1CCc2c([nH]c3ccc(Cl)cc23)C1c1ccc(OCC2CCCCN2C)cc1] | 1010 |
| 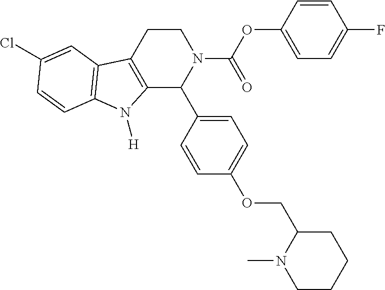 [SMILES: CN1CCCCC1COc1ccc(C2c3[nH]c4ccc(Cl)cc4c3CCN2C(=O)Oc2ccc(F)cc2)cc1] | 1011 |
| 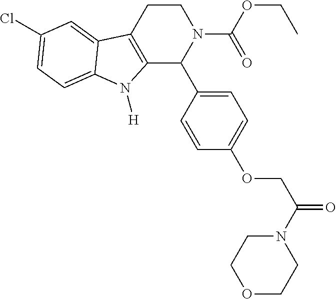 [SMILES: CCOC(=O)N1CCc2c([nH]c3ccc(Cl)cc23)C1c1ccc(OCC(=O)N2CCOCC2)cc1] | 1012 |
| 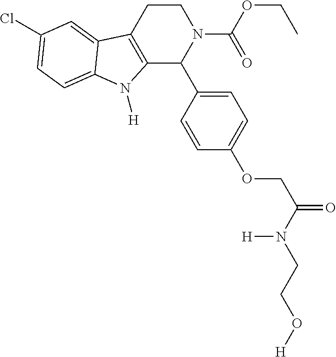 [SMILES: CCOC(=O)N1CCc2c([nH]c3ccc(Cl)cc23)C1c1ccc(OCC(=O)NCCO)cc1] | 1013 |
| 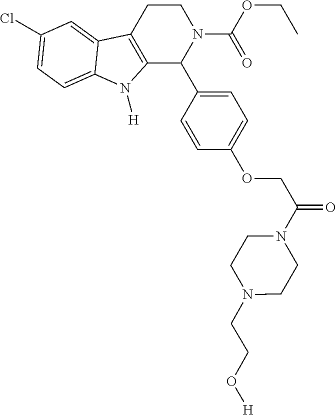 [SMILES: CCOC(=O)N1CCc2c([nH]c3ccc(Cl)cc23)C1c1ccc(OCC(=O)N2CCN(CCO)CC2)cc1] | 1014 |
| 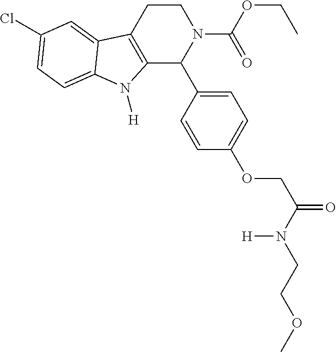 [SMILES: CCOC(=O)N1CCc2c([nH]c3ccc(Cl)cc23)C1c1ccc(OCC(=O)NCCOC)cc1] | 1015 |
| 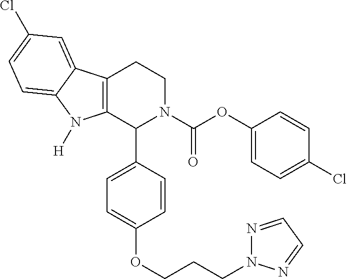 [SMILES: O=C(Oc1ccc(Cl)cc1)N1CCc2c([nH]c3ccc(Cl)cc23)C1c1ccc(OCCCn2nccn2)cc1] | 1016 |
| 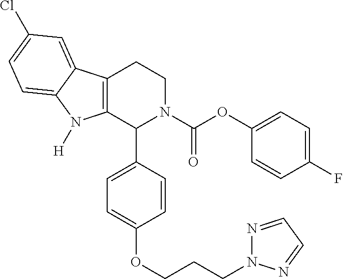 [SMILES: O=C(Oc1ccc(F)cc1)N1CCc2c([nH]c3ccc(Cl)cc23)C1c1ccc(OCCCn2nccn2)cc1] | 1017 |
| 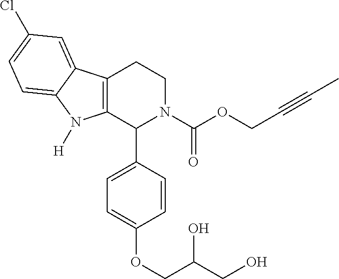 [SMILES: CC#CCOC(=O)N1CCc2c([nH]c3ccc(Cl)cc23)C1c1ccc(OCC(O)CO)cc1] | 1018 |
| 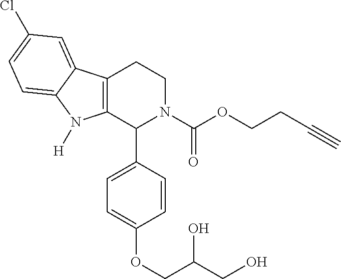 [SMILES: C#CCCOC(=O)N1CCc2c([nH]c3ccc(Cl)cc23)C1c1ccc(OCC(O)CO)cc1] | 1019 |
| 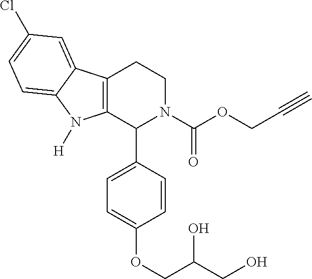 [SMILES: C#CCOC(=O)N1CCc2c([nH]c3ccc(Cl)cc23)C1c1ccc(OCC(O)CO)cc1] | 1020 |
| 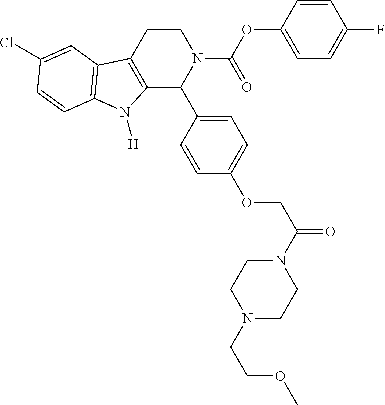 [SMILES: COCCN1CCN(C(=O)COc2ccc(C3c4[nH]c5ccc(Cl)cc5c4CCN3C(=O)Oc3ccc(F)cc3)cc2)CC1] | 1021 |
| 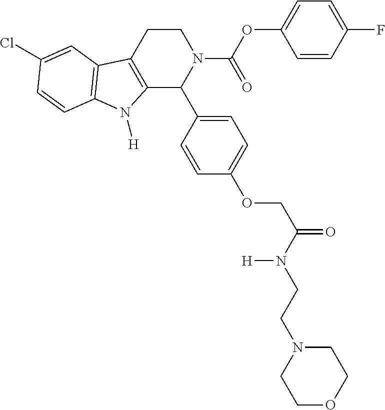 [SMILES: O=C(COc1ccc(C2c3[nH]c4ccc(Cl)cc4c3CCN2C(=O)Oc2ccc(F)cc2)cc1)NCCN1CCOCC1] | 1022 |
| 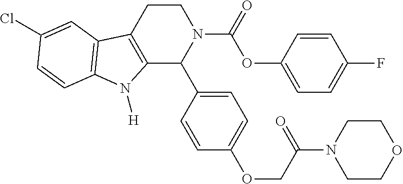 [SMILES: O=C(COc1ccc(C2c3[nH]c4ccc(Cl)cc4c3CCN2C(=O)Oc2ccc(F)cc2)cc1)N1CCOCC1] | 1023 |
| 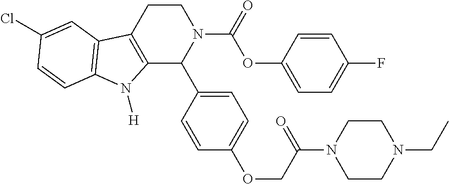 [SMILES: CCN1CCN(C(=O)COc2ccc(C3c4[nH]c5ccc(Cl)cc5c4CCN3C(=O)Oc3ccc(F)cc3)cc2)CC1] | 1024 |
| 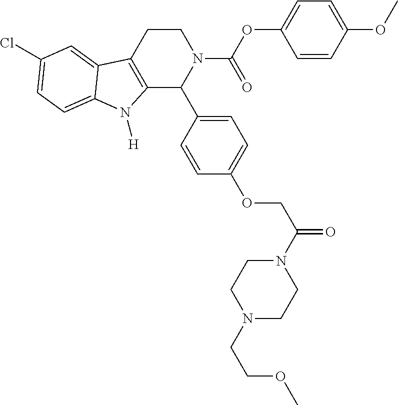 [SMILES: COCCN1CCN(C(=O)COc2ccc(C3c4[nH]c5ccc(Cl)cc5c4CCN3C(=O)Oc3ccc(OC)cc3)cc2)CC1] | 1025 |
| 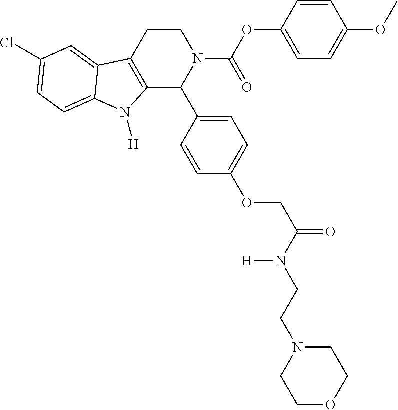 [SMILES: COc1ccc(OC(=O)N2CCc3c([nH]c4ccc(Cl)cc34)C2c2ccc(OCC(=O)NCCN3CCOCC3)cc2)cc1] | 1026 |
| 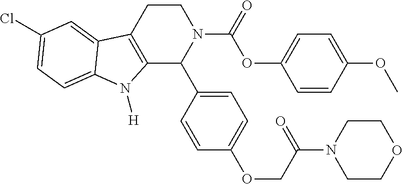 [SMILES: COc1ccc(OC(=O)N2CCc3c([nH]c4ccc(Cl)cc34)C2c2ccc(OCC(=O)N3CCOCC3)cc2)cc1] | 1027 |
| 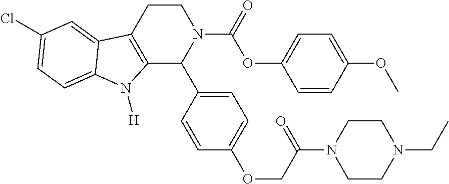 [SMILES: CCN1CCN(C(=O)COc2ccc(C3c4[nH]c5ccc(Cl)cc5c4CCN3C(=O)Oc3ccc(OC)cc3)cc2)CC1] | 1028 |
| 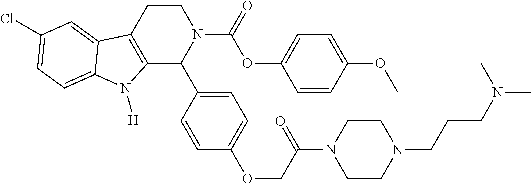 [SMILES: COc1ccc(OC(=O)N2CCc3c([nH]c4ccc(Cl)cc34)C2c2ccc(OCC(=O)N3CCN(CCCN(C)C)CC3)cc2)cc1] | 1029 |
| 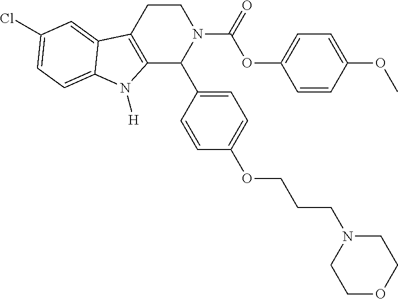 [SMILES: COc1ccc(OC(=O)N2CCc3c([nH]c4ccc(Cl)cc34)C2c2ccc(OCCCN3CCOCC3)cc2)cc1] | 1030 |
| 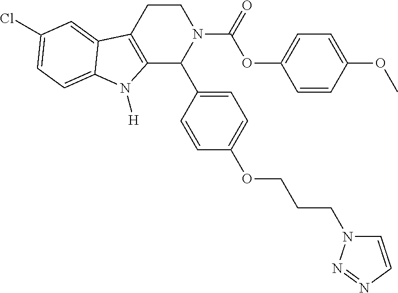 [SMILES: COc1ccc(OC(=O)N2CCc3c([nH]c4ccc(Cl)cc34)C2c2ccc(OCCCn3ccnn3)cc2)cc1] | 1031 |
| 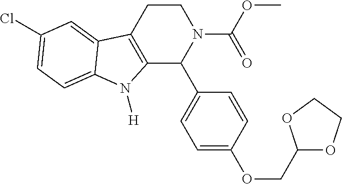 [SMILES: COC(=O)N1CCc2c([nH]c3ccc(Cl)cc23)C1c1ccc(OCC2OCCO2)cc1] | 1032 |
| 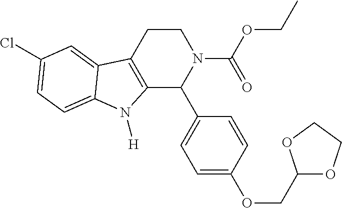 [SMILES: CCOC(=O)N1CCc2c([nH]c3ccc(Cl)cc23)C1c1ccc(OCC2OCCO2)cc1] | 1033 |
| 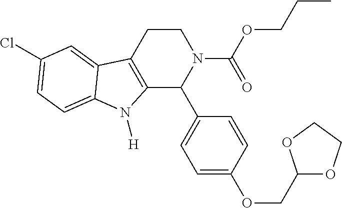 [SMILES: CCCOC(=O)N1CCc2c([nH]c3ccc(Cl)cc23)C1c1ccc(OCC2OCCO2)cc1] | 1034 |
| 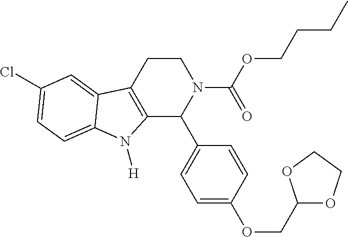 [SMILES: CCCCOC(=O)N1CCc2c([nH]c3ccc(Cl)cc23)C1c1ccc(OCC2OCCO2)cc1] | 1035 |
| 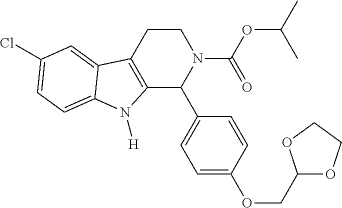 [SMILES: CC(C)OC(=O)N1CCc2c([nH]c3ccc(Cl)cc23)C1c1ccc(OCC2OCCO2)cc1] | 1036 |
| 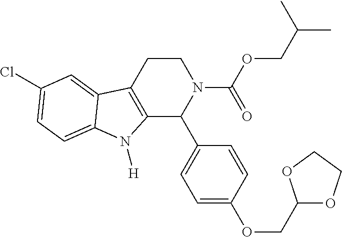 [SMILES: CC(C)COC(=O)N1CCc2c([nH]c3ccc(Cl)cc23)C1c1ccc(OCC2OCCO2)cc1] | 1037 |
| 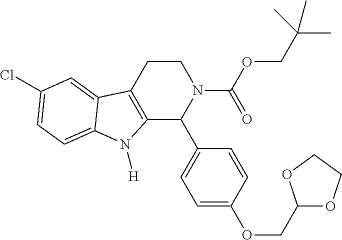 [SMILES: CC(C)(C)COC(=O)N1CCc2c([nH]c3ccc(Cl)cc23)C1c1ccc(OCC2OCCO2)cc1] | 1038 |
| 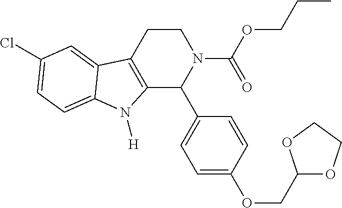 [SMILES: CCCOC(=O)N1CCc2c([nH]c3ccc(Cl)cc23)C1c1ccc(OCC2OCCO2)cc1] | 1039 |
| 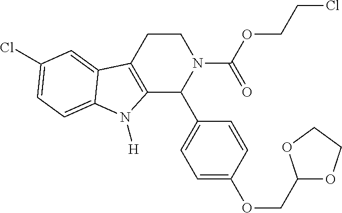 [SMILES: O=C(OCCCl)N1CCc2c([nH]c3ccc(Cl)cc23)C1c1ccc(OCC2OCCO2)cc1] | 1040 |
| 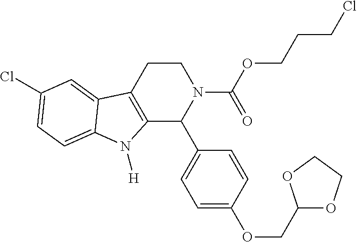 [SMILES: O=C(OCCCCl)N1CCc2c([nH]c3ccc(Cl)cc23)C1c1ccc(OCC2OCCO2)cc1] | 1041 |
| 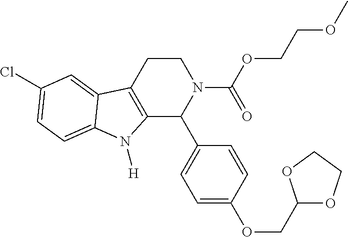 [SMILES: COCCOC(=O)N1CCc2c([nH]c3ccc(Cl)cc23)C1c1ccc(OCC2OCCO2)cc1] | 1042 |
| 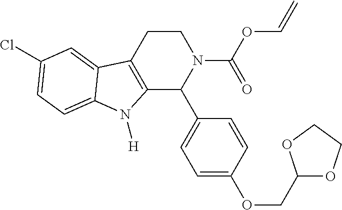 [SMILES: C=COC(=O)N1CCc2c([nH]c3ccc(Cl)cc23)C1c1ccc(OCC2OCCO2)cc1] | 1043 |
| 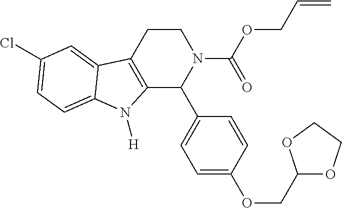 [SMILES: C=CCOC(=O)N1CCc2c([nH]c3ccc(Cl)cc23)C1c1ccc(OCC2OCCO2)cc1] | 1044 |
| 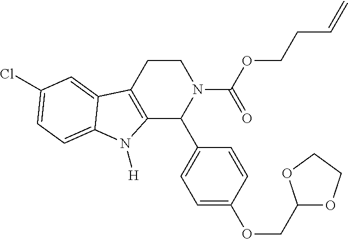 [SMILES: C=CCCOC(=O)N1CCc2c([nH]c3ccc(Cl)cc23)C1c1ccc(OCC2OCCO2)cc1] | 1045 |
| 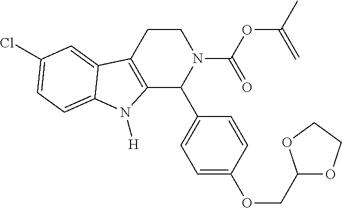 [SMILES: C=C(C)OC(=O)N1CCc2c([nH]c3ccc(Cl)cc23)C1c1ccc(OCC2OCCO2)cc1] | 1046 |
| 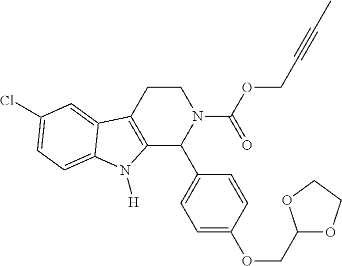 [SMILES: CC#CCOC(=O)N1CCc2c([nH]c3ccc(Cl)cc23)C1c1ccc(OCC2OCCO2)cc1] | 1047 |
| 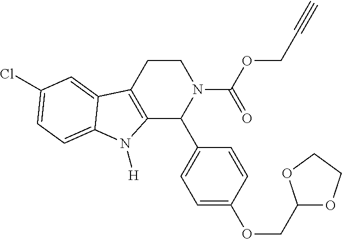 [SMILES: C#CCOC(=O)N1CCc2c([nH]c3ccc(Cl)cc23)C1c1ccc(OCC2OCCO2)cc1] | 1048 |
| 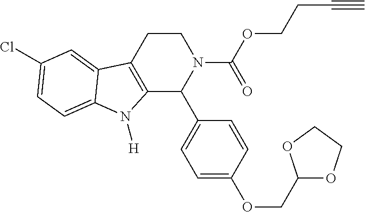 [SMILES: C#CCCOC(=O)N1CCc2c([nH]c3ccc(Cl)cc23)C1c1ccc(OCC2OCCO2)cc1] | 1049 |
| 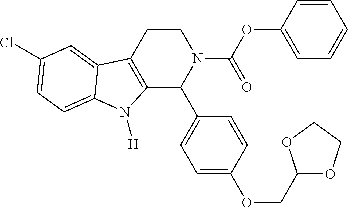 [SMILES: O=C(Oc1ccccc1)N1CCc2c([nH]c3ccc(Cl)cc23)C1c1ccc(OCC2OCCO2)cc1] | 1050 |
| 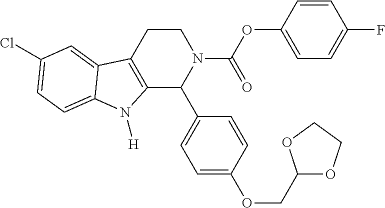 [SMILES: O=C(Oc1ccc(F)cc1)N1CCc2c([nH]c3ccc(Cl)cc23)C1c1ccc(OCC2OCCO2)cc1] | 1051 |
| 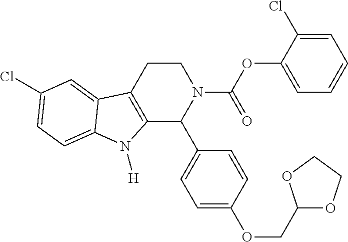 [SMILES: O=C(Oc1ccccc1Cl)N1CCc2c([nH]c3ccc(Cl)cc23)C1c1ccc(OCC2OCCO2)cc1] | 1052 |
| 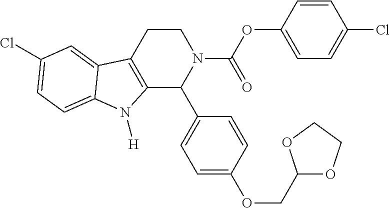 [SMILES: O=C(Oc1ccc(Cl)cc1)N1CCc2c([nH]c3ccc(Cl)cc23)C1c1ccc(OCC2OCCO2)cc1] | 1053 |
| 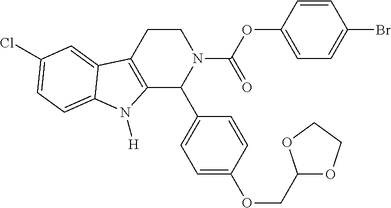 [SMILES: O=C(Oc1ccc(Br)cc1)N1CCc2c([nH]c3ccc(Cl)cc23)C1c1ccc(OCC2OCCO2)cc1] | 1054 |
| 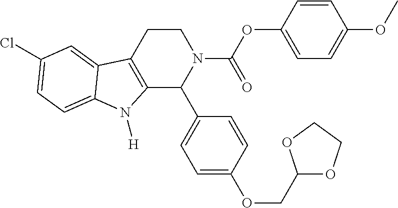 [SMILES: COc1ccc(OC(=O)N2CCc3c([nH]c4ccc(Cl)cc34)C2c2ccc(OCC3OCCO3)cc2)cc1] | 1055 |
| 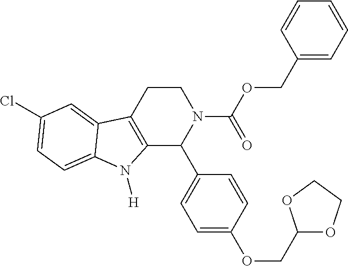 [SMILES: O=C(OCc1ccccc1)N1CCc2c([nH]c3ccc(Cl)cc23)C1c1ccc(OCC2OCCO2)cc1] | 1056 |
| 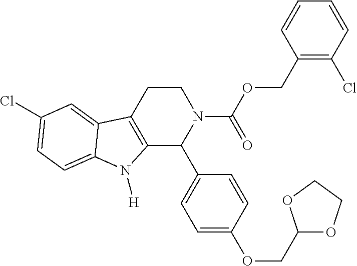 [SMILES: O=C(OCc1ccccc1Cl)N1CCc2c([nH]c3ccc(Cl)cc23)C1c1ccc(OCC2OCCO2)cc1] | 1057 |
| 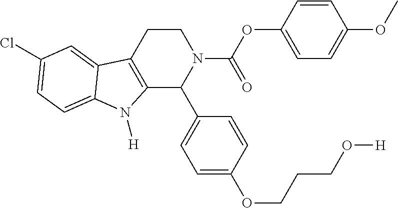 [SMILES: COc1ccc(OC(=O)N2CCc3c([nH]c4ccc(Cl)cc34)C2c2ccc(OCCCO)cc2)cc1] | 1058 |
| 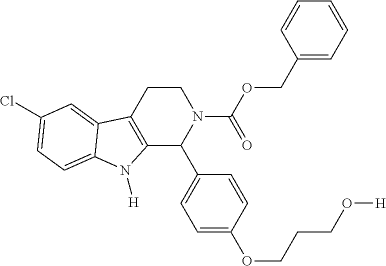 [SMILES: O=C(OCc1ccccc1)N1CCc2c([nH]c3ccc(Cl)cc23)C1c1ccc(OCCCO)cc1] | 1059 |
| 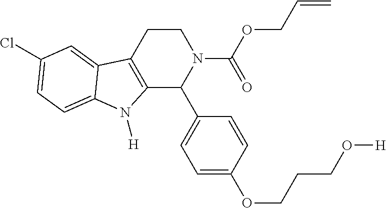 [SMILES: C=CCOC(=O)N1CCc2c([nH]c3ccc(Cl)cc23)C1c1ccc(OCCCO)cc1] | 1060 |
| 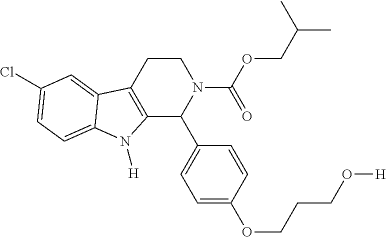 [SMILES: CC(C)COC(=O)N1CCc2c([nH]c3ccc(Cl)cc23)C1c1ccc(OCCCO)cc1] | 1061 |
| 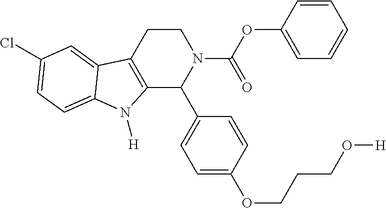 [SMILES: O=C(Oc1ccccc1)N1CCc2c([nH]c3ccc(Cl)cc23)C1c1ccc(OCCCO)cc1] | 1062 |
| 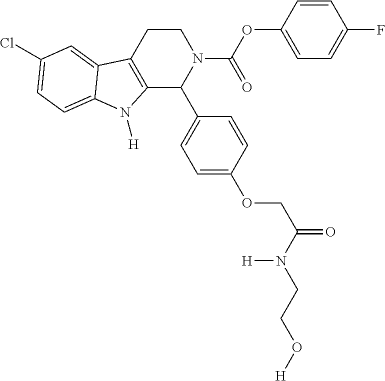 [SMILES: O=C(COc1ccc(C2c3[nH]c4ccc(Cl)cc4c3CCN2C(=O)Oc2ccc(F)cc2)cc1)NCCO] | 1063 |
| 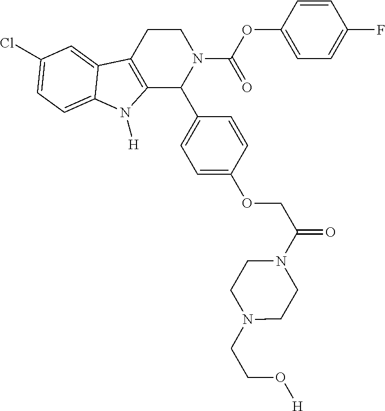 [SMILES: O=C(COc1ccc(C2c3[nH]c4ccc(Cl)cc4c3CCN2C(=O)Oc2ccc(F)cc2)cc1)N1CCN(CCO)CC1] | 1064 |
| 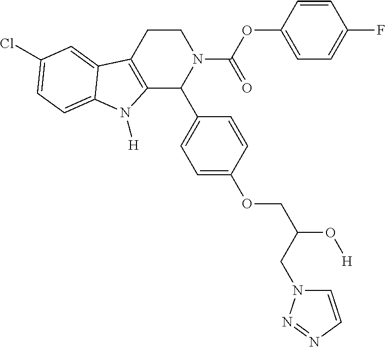 [SMILES: O=C(Oc1ccc(F)cc1)N1CCc2c([nH]c3ccc(Cl)cc23)C1c1ccc(OCC(O)Cn2ccnn2)cc1] | 1066 |
| 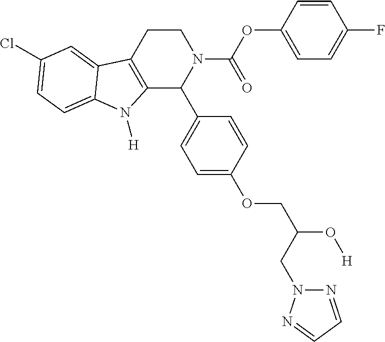 [SMILES: O=C(Oc1ccc(F)cc1)N1CCc2c([nH]c3ccc(Cl)cc23)C1c1ccc(OCC(O)Cn2nccn2)cc1] | 1067 |
| 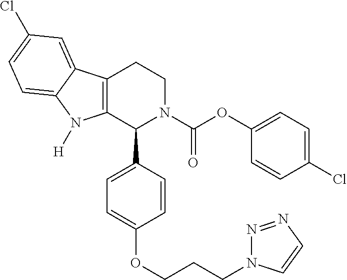 [SMILES: O=C(Oc1ccc(Cl)cc1)N1CCc2c([nH]c3ccc(Cl)cc23)[C@@H]1c1ccc(OCCCn2ccnn2)cc1] | 1068 |
| 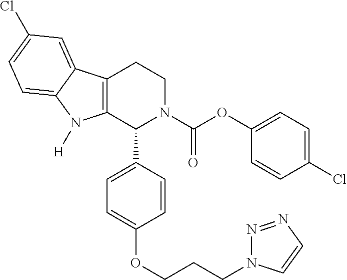 [SMILES: O=C(Oc1ccc(Cl)cc1)N1CCc2c([nH]c3ccc(Cl)cc23)[C@H]1c1ccc(OCCCn2ccnn2)cc1] | 1069 |
| 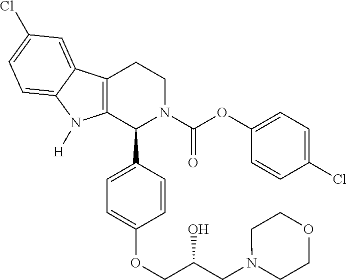 [SMILES: O=C(Oc1ccc(Cl)cc1)N1CCc2c([nH]c3ccc(Cl)cc23)[C@@H]1c1ccc(OC[C@H](O)CN2CCOCC2)cc1] | 1070 |
| 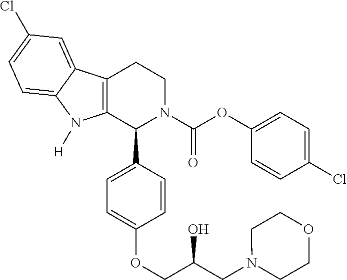 [SMILES: O=C(Oc1ccc(Cl)cc1)N1CCc2c([nH]c3ccc(Cl)cc23)[C@@H]1c1ccc(OC[C@@H](O)CN2CCOCC2)cc1] | 1071 |
| 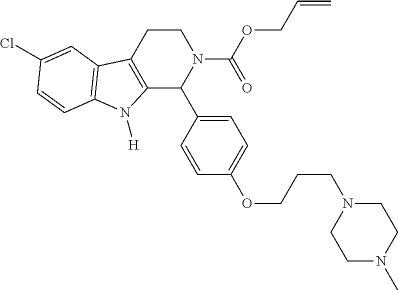 [SMILES: C=CCOC(=O)N1CCc2c([nH]c3ccc(Cl)cc23)C1c1ccc(OCCCN2CCN(C)CC2)cc1] | 1072 |
| 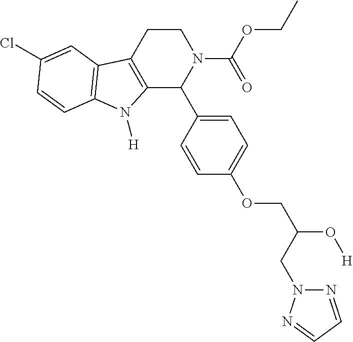 [SMILES: CCOC(=O)N1CCc2c([nH]c3ccc(Cl)cc23)C1c1ccc(OCC(O)Cn2nccn2)cc1] | 1073 |
| 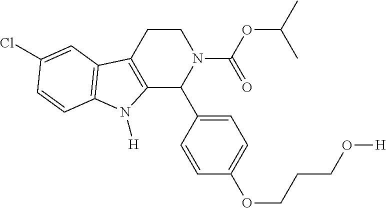 [SMILES: CC(C)OC(=O)N1CCc2c([nH]c3ccc(Cl)cc23)C1c1ccc(OCCCO)cc1] | 1074 |
| 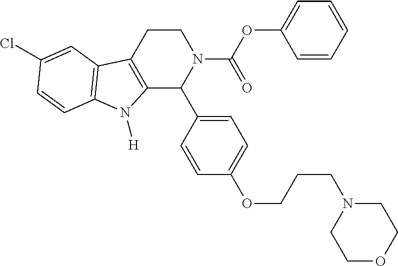 [SMILES: O=C(Oc1ccccc1)N1CCc2c([nH]c3ccc(Cl)cc23)C1c1ccc(OCCCN2CCOCC2)cc1] | 1075 |
| 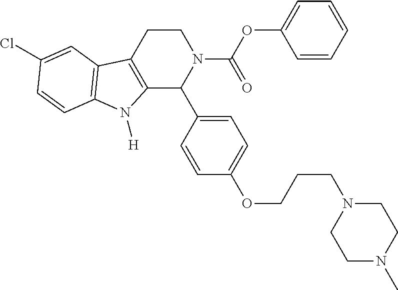 [SMILES: CN1CCN(CCCOc2ccc(C3c4[nH]c5ccc(Cl)cc5c4CCN3C(=O)Oc3ccccc3)cc2)CC1] | 1076 |
| 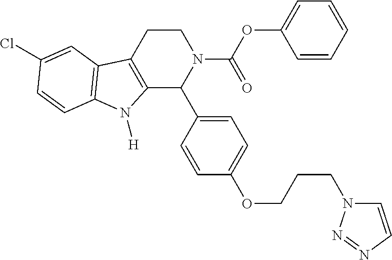 [SMILES: O=C(Oc1ccccc1)N1CCc2c([nH]c3ccc(Cl)cc23)C1c1ccc(OCCCn2ccnn2)cc1] | 1077 |
| 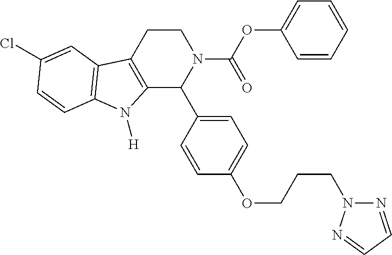 [SMILES: O=C(Oc1ccccc1)N1CCc2c([nH]c3ccc(Cl)cc23)C1c1ccc(OCCCn2nccn2)cc1] | 1078 |
| 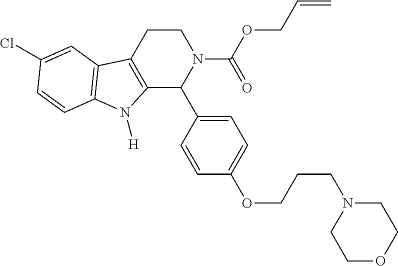 [SMILES: C=CCOC(=O)N1CCc2c([nH]c3ccc(Cl)cc23)C1c1ccc(OCCCN2CCOCC2)cc1] | 1079 |
| 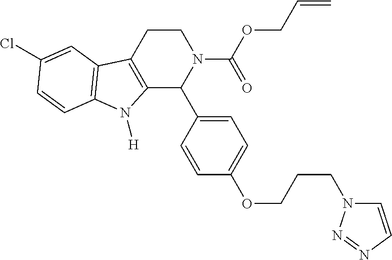 [SMILES: C=CCOC(=O)N1CCc2c([nH]c3ccc(Cl)cc23)C1c1ccc(OCCCn2ccnn2)cc1] | 1080 |
| 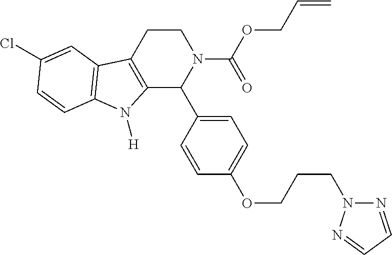 [SMILES: C=CCOC(=O)N1CCc2c([nH]c3ccc(Cl)cc23)C1c1ccc(OCCCn2nccn2)cc1] | 1081 |
| 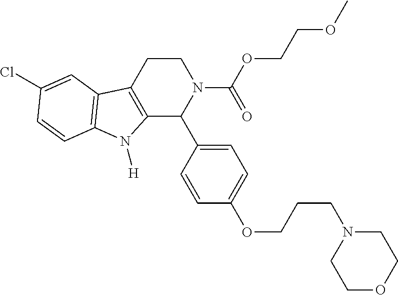 [SMILES: COCCOC(=O)N1CCc2c([nH]c3ccc(Cl)cc23)C1c1ccc(OCCCN2CCOCC2)cc1] | 1082 |
| 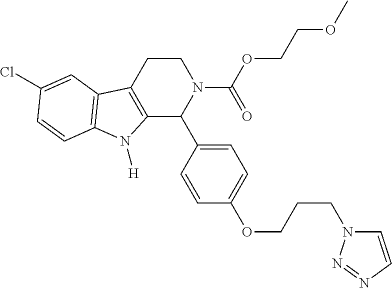 [SMILES: COCCOC(=O)N1CCc2c([nH]c3ccc(Cl)cc23)C1c1ccc(OCCCn2ccnn2)cc1] | 1083 |
| 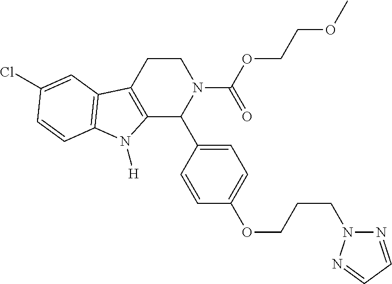 [SMILES: COCCOC(=O)N1CCc2c([nH]c3ccc(Cl)cc23)C1c1ccc(OCCCn2nccn2)cc1] | 1084 |
| 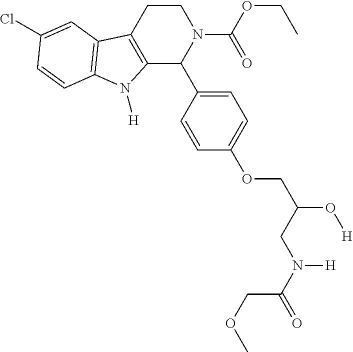 [SMILES: CCOC(=O)N1CCc2c([nH]c3ccc(Cl)cc23)C1c1ccc(OCC(O)CNC(=O)COC)cc1] | 1085 |
| 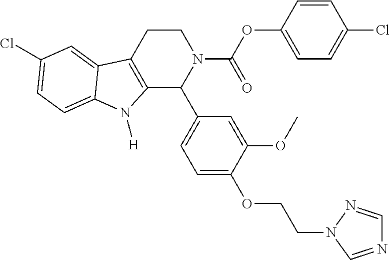 [SMILES: COc1cc(C2c3[nH]c4ccc(Cl)cc4c3CCN2C(=O)Oc2ccc(Cl)cc2)ccc1OCCn1cncn1] | 1086 |
| 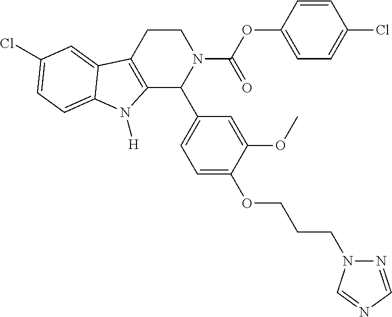 [SMILES: COc1cc(C2c3[nH]c4ccc(Cl)cc4c3CCN2C(=O)Oc2ccc(Cl)cc2)ccc1OCCCn1cncn1] | 1087 |
| 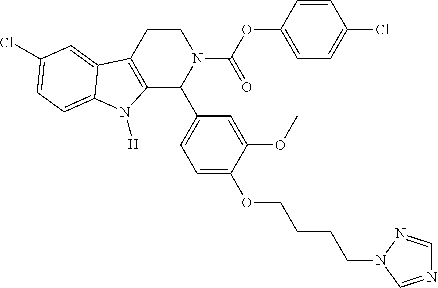 [SMILES: COc1cc(C2c3[nH]c4ccc(Cl)cc4c3CCN2C(=O)Oc2ccc(Cl)cc2)ccc1OCCCCn1cncn1] | 1088 |
| 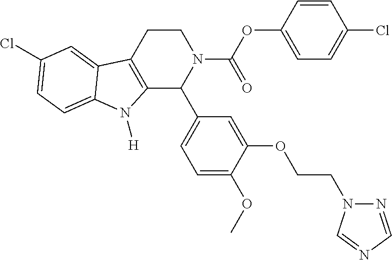 [SMILES: COc1ccc(C2c3[nH]c4ccc(Cl)cc4c3CCN2C(=O)Oc2ccc(Cl)cc2)cc1OCCn1cncn1] | 1089 |
| 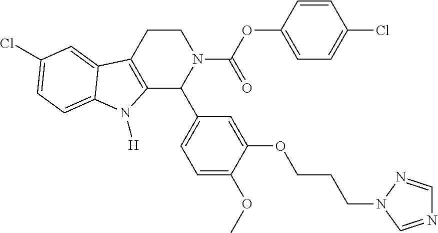 [SMILES: COc1ccc(C2c3[nH]c4ccc(Cl)cc4c3CCN2C(=O)Oc2ccc(Cl)cc2)cc1OCCCn1cncn1] | 1090 |
| 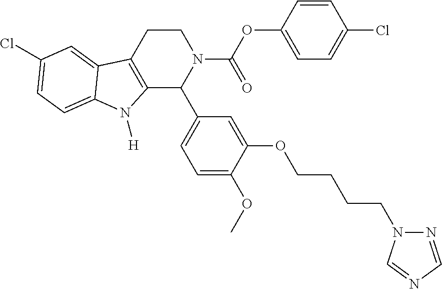 [SMILES: COc1ccc(C2c3[nH]c4ccc(Cl)cc4c3CCN2C(=O)Oc2ccc(Cl)cc2)cc1OCCCCn1cncn1] | 1091 |
| 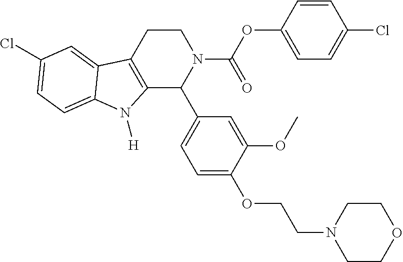 [SMILES: COc1cc(C2c3[nH]c4ccc(Cl)cc4c3CCN2C(=O)Oc2ccc(Cl)cc2)ccc1OCCN1CCOCC1] | 1092 |
| 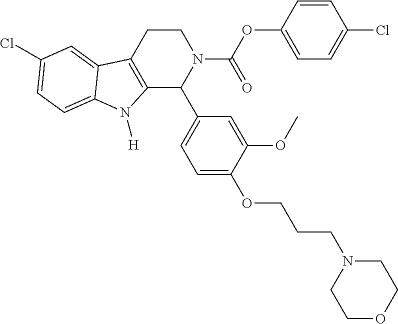 [SMILES: COc1cc(C2c3[nH]c4ccc(Cl)cc4c3CCN2C(=O)Oc2ccc(Cl)cc2)ccc1OCCCN1CCOCC1] | 1093 |
| 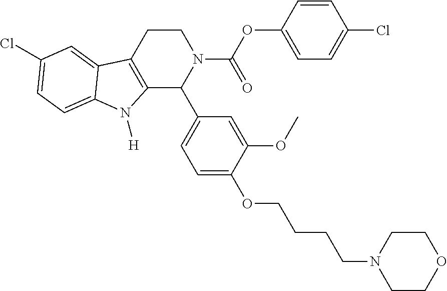 [SMILES: COc1cc(C2c3[nH]c4ccc(Cl)cc4c3CCN2C(=O)Oc2ccc(Cl)cc2)ccc1OCCCCN1CCOCC1] | 1094 |
| 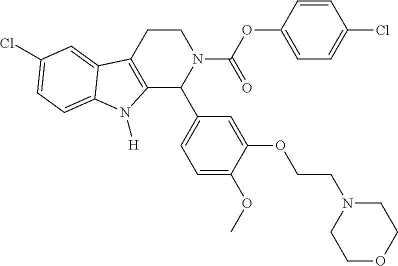 [SMILES: COc1ccc(C2c3[nH]c4ccc(Cl)cc4c3CCN2C(=O)Oc2ccc(Cl)cc2)cc1OCCN1CCOCC1] | 1095 |
| 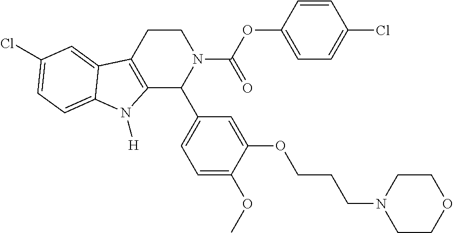 [SMILES: COc1ccc(C2c3[nH]c4ccc(Cl)cc4c3CCN2C(=O)Oc2ccc(Cl)cc2)cc1OCCCN1CCOCC1] | 1096 |
| 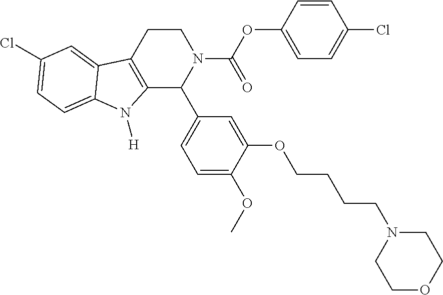 [SMILES: COc1ccc(C2c3[nH]c4ccc(Cl)cc4c3CCN2C(=O)Oc2ccc(Cl)cc2)cc1OCCCCN1CCOCC1] | 1097 |
| 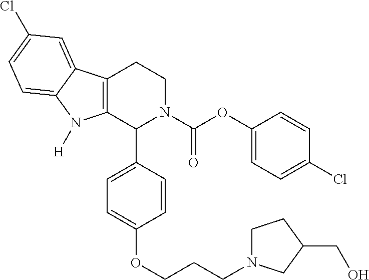 [SMILES: O=C(Oc1ccc(Cl)cc1)N1CCc2c([nH]c3ccc(Cl)cc23)C1c1ccc(OCCCN2CCC(CO)C2)cc1] | 1098 |
| 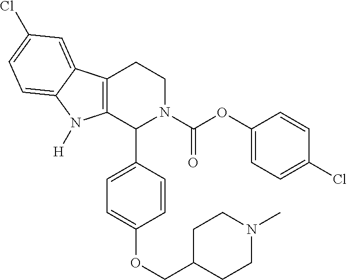 [SMILES: CN1CCC(COc2ccc(C3c4[nH]c5ccc(Cl)cc5c4CCN3C(=O)Oc3ccc(Cl)cc3)cc2)CC1] | 1099 |
| 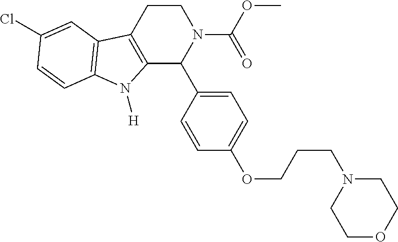 [SMILES: COC(=O)N1CCc2c([nH]c3ccc(Cl)cc23)C1c1ccc(OCCCN2CCOCC2)cc1] | 1100 |
| 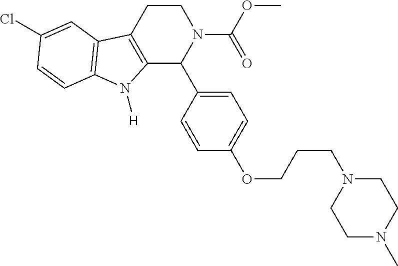 [SMILES: COC(=O)N1CCc2c([nH]c3ccc(Cl)cc23)C1c1ccc(OCCCN2CCN(C)CC2)cc1] | 1101 |
| 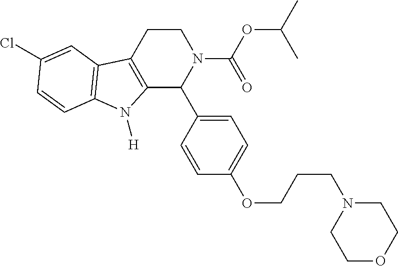 [SMILES: CC(C)OC(=O)N1CCc2c([nH]c3ccc(Cl)cc23)C1c1ccc(OCCCN2CCOCC2)cc1] | 1102 |
| 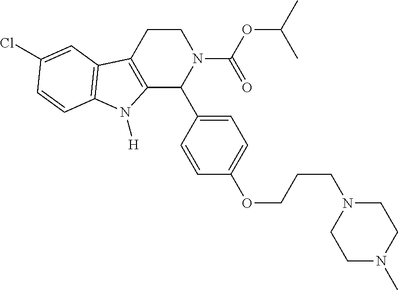 [SMILES: CC(C)OC(=O)N1CCc2c([nH]c3ccc(Cl)cc23)C1c1ccc(OCCCN2CCN(C)CC2)cc1] | 1103 |
| 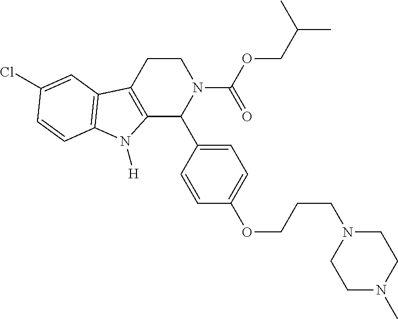 [SMILES: CC(C)COC(=O)N1CCc2c([nH]c3ccc(Cl)cc23)C1c1ccc(OCCCN2CCN(C)CC2)cc1] | 1104 |
| 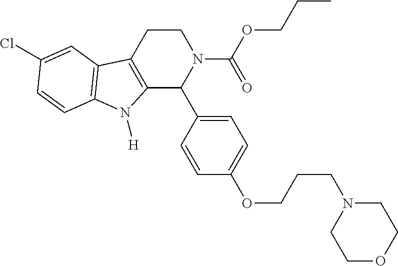 [SMILES: CCCOC(=O)N1CCc2c([nH]c3ccc(Cl)cc23)C1c1ccc(OCCCN2CCOCC2)cc1] | 1105 |
| 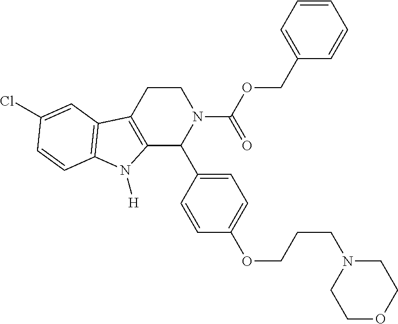 [SMILES: O=C(OCc1ccccc1)N1CCc2c([nH]c3ccc(Cl)cc23)C1c1ccc(OCCCN2CCOCC2)cc1] | 1106 |
| 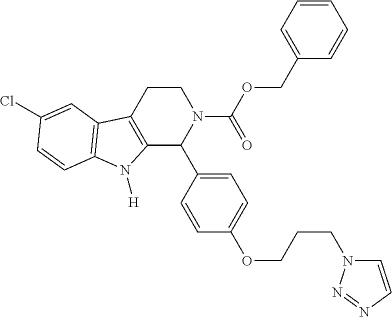 [SMILES: O=C(OCc1ccccc1)N1CCc2c([nH]c3ccc(Cl)cc23)C1c1ccc(OCCCn2ccnn2)cc1] | 1107 |
| 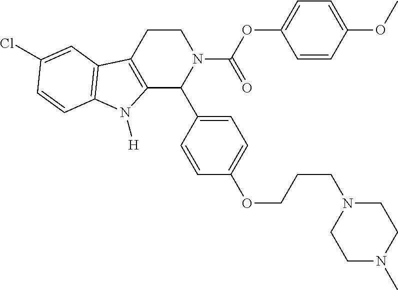 [SMILES: COc1ccc(OC(=O)N2CCc3c([nH]c4ccc(Cl)cc34)C2c2ccc(OCCCN3CCN(C)CC3)cc2)cc1] | 1108 |
| 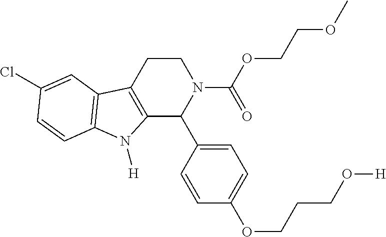 [SMILES: COCCOC(=O)N1CCc2c([nH]c3ccc(Cl)cc23)C1c1ccc(OCCCO)cc1] | 1109 |
| 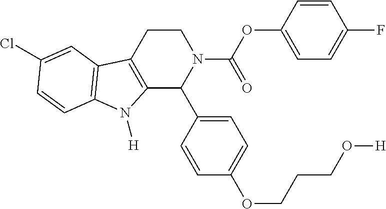 [SMILES: O=C(Oc1ccc(F)cc1)N1CCc2c([nH]c3ccc(Cl)cc23)C1c1ccc(OCCCO)cc1] | 1110 |
| 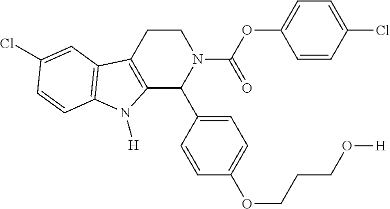 [SMILES: O=C(Oc1ccc(Cl)cc1)N1CCc2c([nH]c3ccc(Cl)cc23)C1c1ccc(OCCCO)cc1] | 1111 |
| 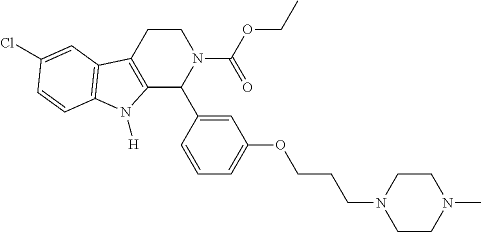 [SMILES: CCOC(=O)N1CCc2c([nH]c3ccc(Cl)cc23)C1c1cccc(OCCCN2CCN(C)CC2)c1] | 1112 |
| 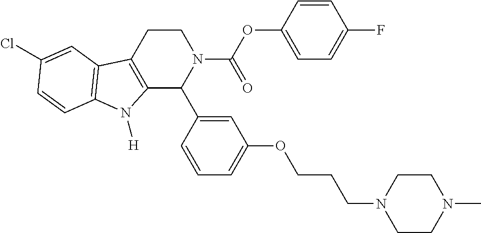 [SMILES: CN1CCN(CCCOc2cccc(C3c4[nH]c5ccc(Cl)cc5c4CCN3C(=O)Oc3ccc(F)cc3)c2)CC1] | 1113 |
| 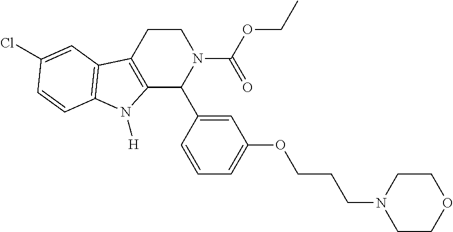 [SMILES: CCOC(=O)N1CCc2c([nH]c3ccc(Cl)cc23)C1c1cccc(OCCCN2CCOCC2)c1] | 1114 |
| 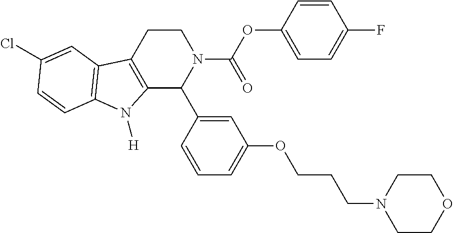 [SMILES: O=C(Oc1ccc(F)cc1)N1CCc2c([nH]c3ccc(Cl)cc23)C1c1cccc(OCCCN2CCOCC2)c1] | 1115 |
| 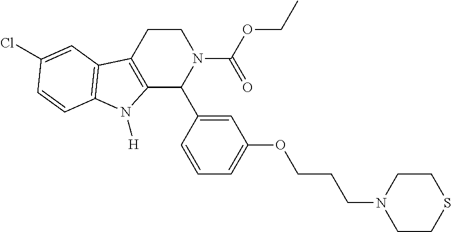 [SMILES: CCOC(=O)N1CCc2c([nH]c3ccc(Cl)cc23)C1c1cccc(OCCCN2CCSCC2)c1] | 1116 |
| 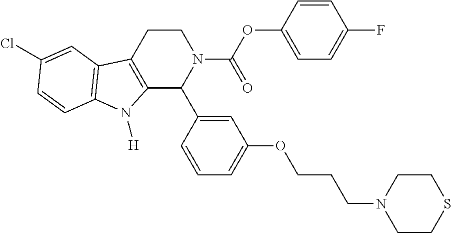 [SMILES: O=C(Oc1ccc(F)cc1)N1CCc2c([nH]c3ccc(Cl)cc23)C1c1cccc(OCCCN2CCSCC2)c1] | 1117 |
| 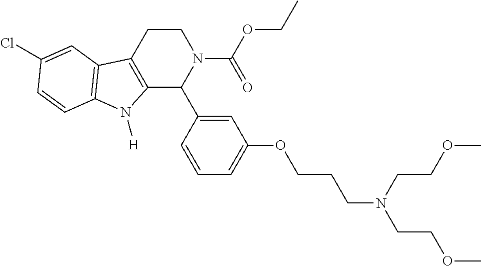 [SMILES: CCOC(=O)N1CCc2c([nH]c3ccc(Cl)cc23)C1c1cccc(OCCCN(CCOC)CCOC)c1] | 1118 |
| 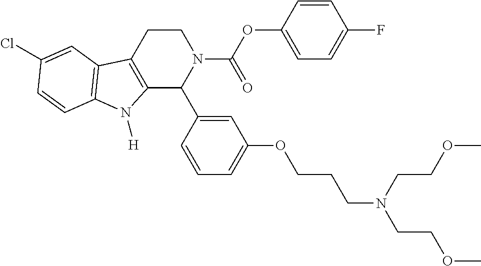 [SMILES: COCCN(CCCOc1cccc(C2c3[nH]c4ccc(Cl)cc4c3CCN2C(=O)Oc2ccc(F)cc2)c1)CCOC] | 1119 |
| 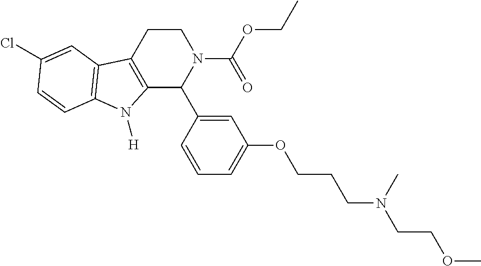 [SMILES: CCOC(=O)N1CCc2c([nH]c3ccc(Cl)cc23)C1c1cccc(OCCCN(C)CCOC)c1] | 1120 |
| 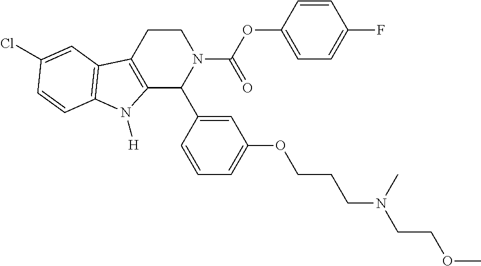 [SMILES: COCCN(C)CCCOc1cccc(C2c3[nH]c4ccc(Cl)cc4c3CCN2C(=O)Oc2ccc(F)cc2)c1] | 1121 |
| 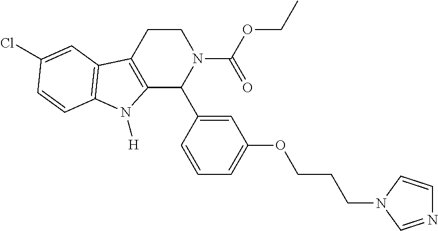 [SMILES: CCOC(=O)N1CCc2c([nH]c3ccc(Cl)cc23)C1c1cccc(OCCCn2ccnc2)c1] | 1122 |
| 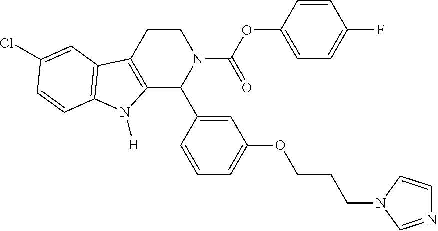 [SMILES: O=C(Oc1ccc(F)cc1)N1CCc2c([nH]c3ccc(Cl)cc23)C1c1cccc(OCCCn2ccnc2)c1] | 1123 |
| 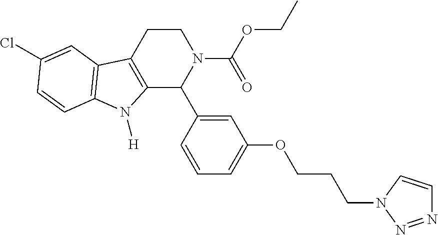 [SMILES: CCOC(=O)N1CCc2c([nH]c3ccc(Cl)cc23)C1c1cccc(OCCCn2ccnn2)c1] | 1124 |
| 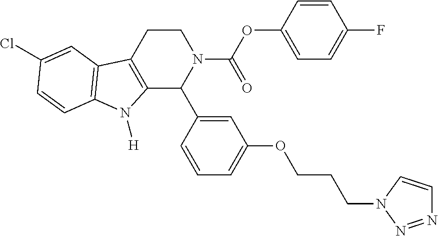 [SMILES: O=C(Oc1ccc(F)cc1)N1CCc2c([nH]c3ccc(Cl)cc23)C1c1cccc(OCCCn2ccnn2)c1] | 1125 |
| 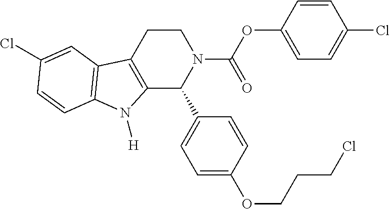 [SMILES: O=C(Oc1ccc(Cl)cc1)N1CCc2c([nH]c3ccc(Cl)cc23)[C@H]1c1ccc(OCCCCl)cc1] | 1126 |
| 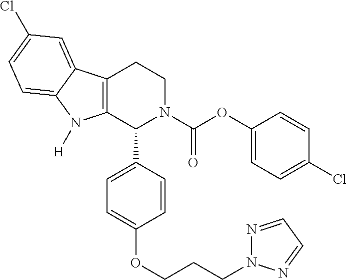 [SMILES: O=C(Oc1ccc(Cl)cc1)N1CCc2c([nH]c3ccc(Cl)cc23)[C@H]1c1ccc(OCCCn2nccn2)cc1] | 1127 |
| 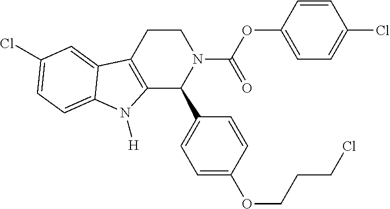 [SMILES: O=C(Oc1ccc(Cl)cc1)N1CCc2c([nH]c3ccc(Cl)cc23)[C@@H]1c1ccc(OCCCCl)cc1] | 1128 |
| 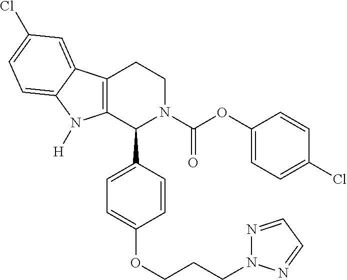 [SMILES: O=C(Oc1ccc(Cl)cc1)N1CCc2c([nH]c3ccc(Cl)cc23)[C@@H]1c1ccc(OCCCn2nccn2)cc1] | 1129 |
| 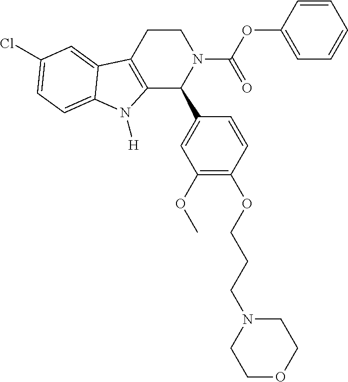 [SMILES: COc1cc([C@H]2c3[nH]c4ccc(Cl)cc4c3CCN2C(=O)Oc2ccccc2)ccc1OCCCN1CCOCC1] | 1130 |
| 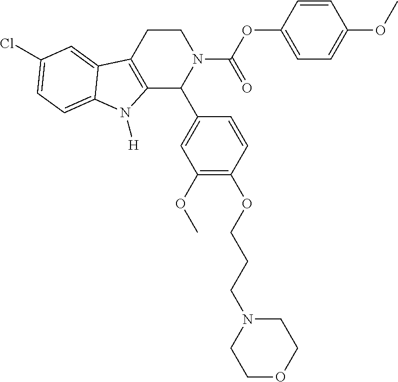 [SMILES: COc1ccc(OC(=O)N2CCc3c([nH]c4ccc(Cl)cc34)C2c2ccc(OCCCN3CCOCC3)c(OC)c2)cc1] | 1131 |
| 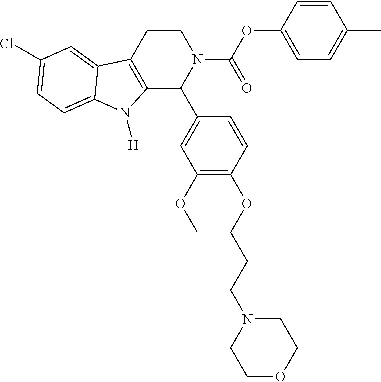 [SMILES: COc1cc(C2c3[nH]c4ccc(Cl)cc4c3CCN2C(=O)Oc2ccc(C)cc2)ccc1OCCCN1CCOCC1] | 1132 |
| 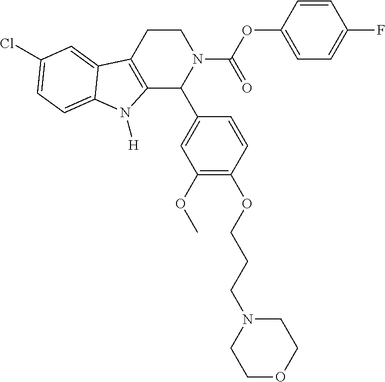 [SMILES: COc1cc(C2c3[nH]c4ccc(Cl)cc4c3CCN2C(=O)Oc2ccc(F)cc2)ccc1OCCCN1CCOCC1] | 1133 |
| 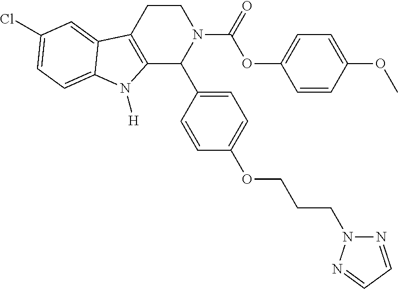 [SMILES: COc1ccc(OC(=O)N2CCc3c([nH]c4ccc(Cl)cc34)C2c2ccc(OCCCn3nccn3)cc2)cc1] | 1134 |
| 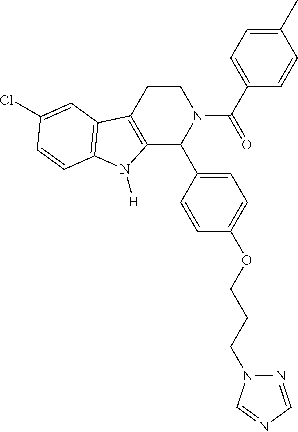 [SMILES: Cc1ccc(C(=O)N2CCc3c([nH]c4ccc(Cl)cc34)C2c2ccc(OCCCn3cncn3)cc2)cc1] | 1135 |
| 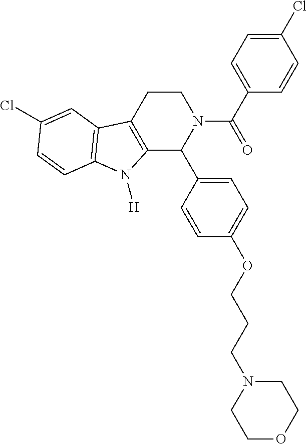 [SMILES: O=C(c1ccc(Cl)cc1)N1CCc2c([nH]c3ccc(Cl)cc23)C1c1ccc(OCCCN2CCOCC2)cc1] | 1136 |
| 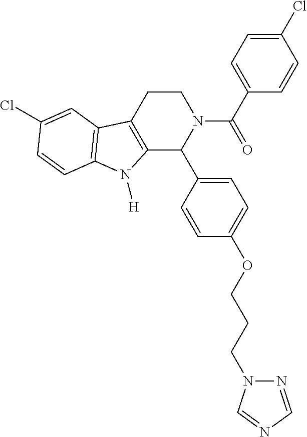 [SMILES: O=C(c1ccc(Cl)cc1)N1CCc2c([nH]c3ccc(Cl)cc23)C1c1ccc(OCCCn2cncn2)cc1] | 1137 |
| 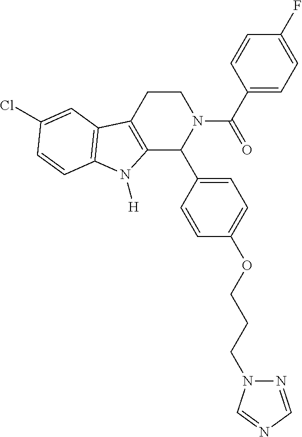 [SMILES: O=C(c1ccc(F)cc1)N1CCc2c([nH]c3ccc(Cl)cc23)C1c1ccc(OCCCn2cncn2)cc1] | 1138 |
| 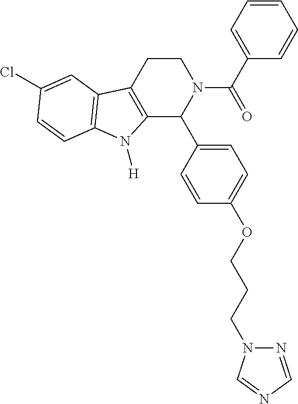 [SMILES: O=C(c1ccccc1)N1CCc2c([nH]c3ccc(Cl)cc23)C1c1ccc(OCCCn2cncn2)cc1] | 1139 |
| 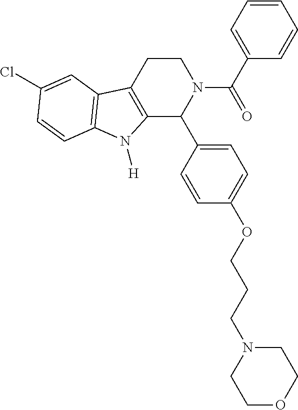 [SMILES: O=C(c1ccccc1)N1CCc2c([nH]c3ccc(Cl)cc23)C1c1ccc(OCCCN2CCOCC2)cc1] | 1140 |
| 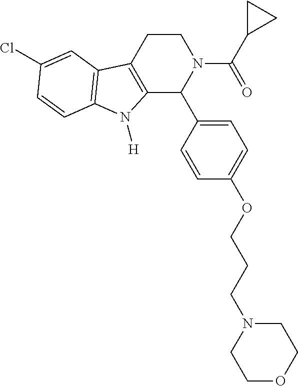 [SMILES: O=C(C1CC1)N1CCc2c([nH]c3ccc(Cl)cc23)C1c1ccc(OCCCN2CCOCC2)cc1] | 1141 |
| 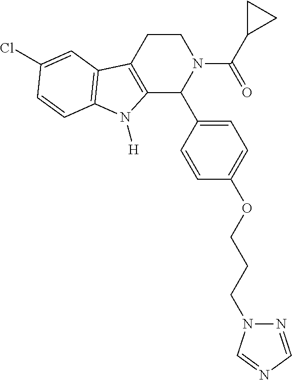 [SMILES: O=C(C1CC1)N1CCc2c([nH]c3ccc(Cl)cc23)C1c1ccc(OCCCn2cncn2)cc1] | 1142 |
| 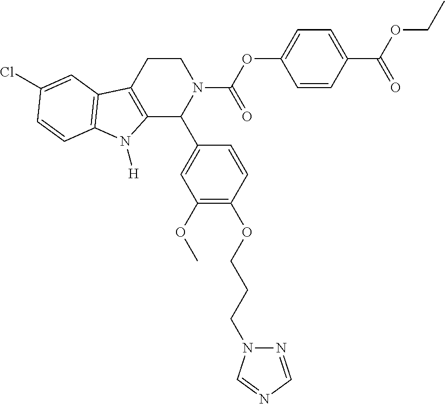 [SMILES: CCOC(=O)c1ccc(OC(=O)N2CCc3c([nH]c4ccc(Cl)cc34)C2c2ccc(OCCCn3cncn3)c(OC)c2)cc1] | 1143 |
| 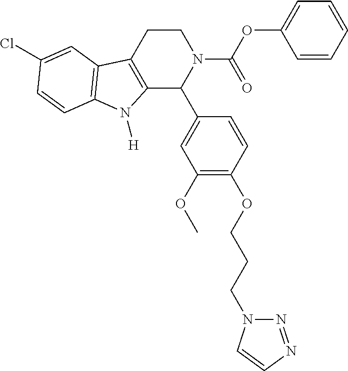 [SMILES: COc1cc(C2c3[nH]c4ccc(Cl)cc4c3CCN2C(=O)Oc2ccccc2)ccc1OCCCn1ccnn1] | 1144 |
| 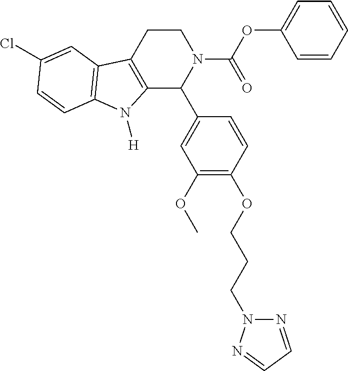 [SMILES: COc1cc(C2c3[nH]c4ccc(Cl)cc4c3CCN2C(=O)Oc2ccccc2)ccc1OCCCn1nccn1] | 1145 |
| 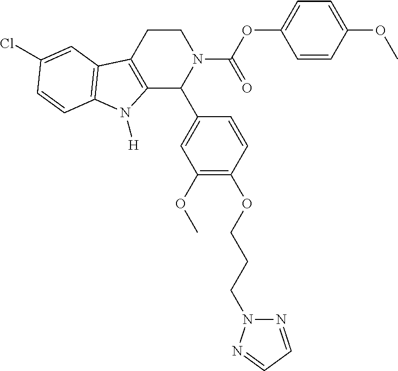 [SMILES: COc1ccc(OC(=O)N2CCc3c([nH]c4ccc(Cl)cc34)C2c2ccc(OCCCn3nccn3)c(OC)c2)cc1] | 1146 |
| 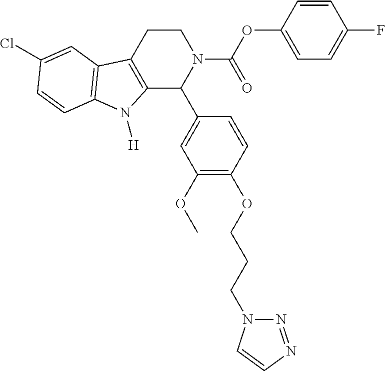 [SMILES: COc1cc(C2c3[nH]c4ccc(Cl)cc4c3CCN2C(=O)Oc2ccc(F)cc2)ccc1OCCCn1ccnn1] | 1147 |
| 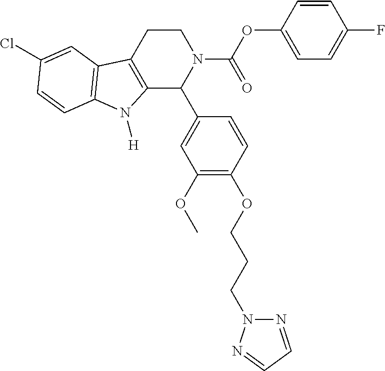 [SMILES: COc1cc(C2c3[nH]c4ccc(Cl)cc4c3CCN2C(=O)Oc2ccc(F)cc2)ccc1OCCCn1nccn1] | 1148 |
| 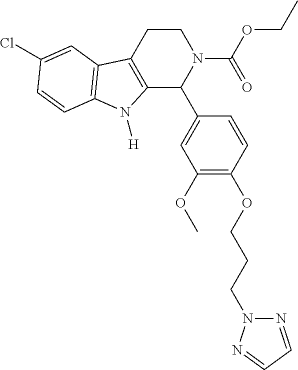 [SMILES: CCOC(=O)N1CCc2c([nH]c3ccc(Cl)cc23)C1c1ccc(OCCCn2nccn2)c(OC)c1] | 1149 |
| 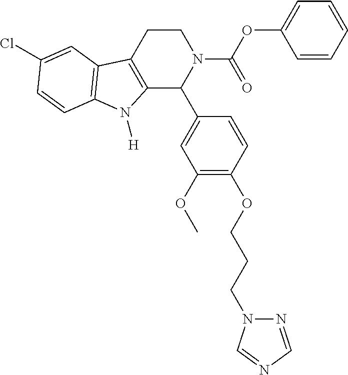 [SMILES: COc1cc(C2c3[nH]c4ccc(Cl)cc4c3CCN2C(=O)Oc2ccccc2)ccc1OCCCn1cncn1] | 1150 |
| 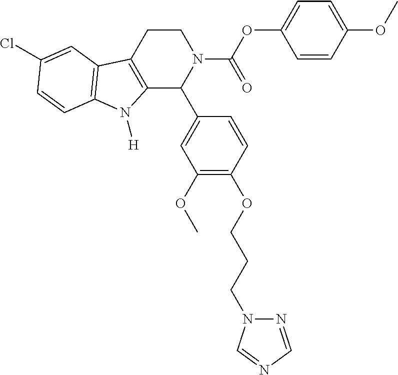 [SMILES: COc1ccc(OC(=O)N2CCc3c([nH]c4ccc(Cl)cc34)C2c2ccc(OCCCn3cncn3)c(OC)c2)cc1] | 1151 |
| 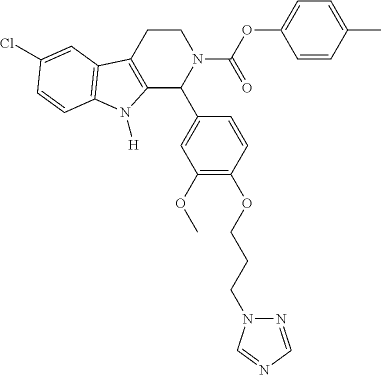 [SMILES: COc1cc(C2c3[nH]c4ccc(Cl)cc4c3CCN2C(=O)Oc2ccc(C)cc2)ccc1OCCCn1cncn1] | 1152 |
| 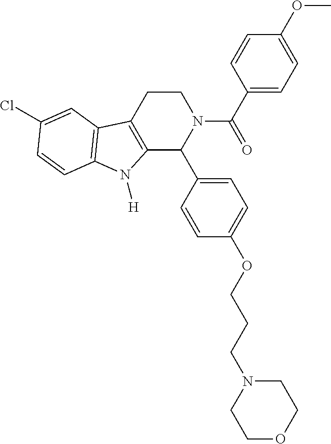 [SMILES: COc1ccc(C(=O)N2CCc3c([nH]c4ccc(Cl)cc34)C2c2ccc(OCCCN3CCOCC3)cc2)cc1] | 1153 |
| 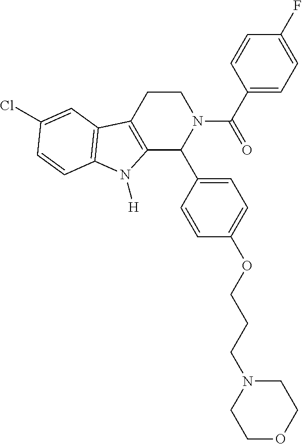 [SMILES: O=C(c1ccc(F)cc1)N1CCc2c([nH]c3ccc(Cl)cc23)C1c1ccc(OCCCN2CCOCC2)cc1] | 1154 |
| 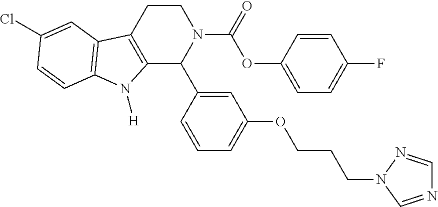 [SMILES: O=C(Oc1ccc(F)cc1)N1CCc2c([nH]c3ccc(Cl)cc23)C1c1cccc(OCCCn2cncn2)c1] | 1155 |
| 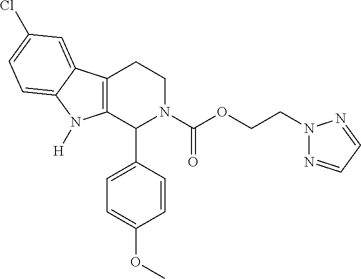 [SMILES: COc1ccc(C2c3[nH]c4ccc(Cl)cc4c3CCN2C(=O)OCCn2nccn2)cc1] | 1156 |
| 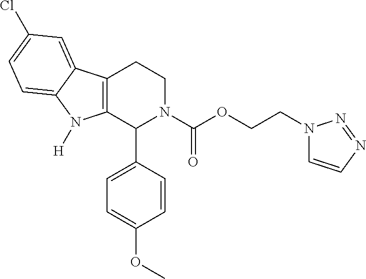 [SMILES: COc1ccc(C2c3[nH]c4ccc(Cl)cc4c3CCN2C(=O)OCCn2ccnn2)cc1] | 1157 |
| 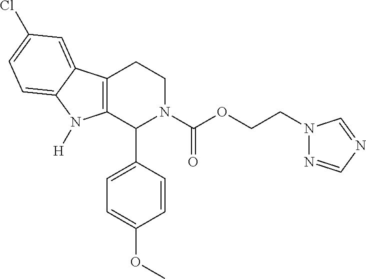 [SMILES: COc1ccc(C2c3[nH]c4ccc(Cl)cc4c3CCN2C(=O)OCCn2cncn2)cc1] | 1158 |
| 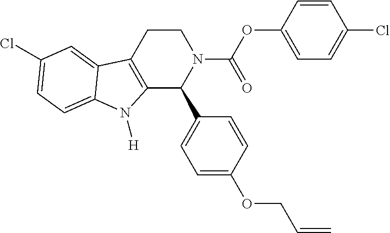 [SMILES: C=CCOc1ccc([C@H]2c3[nH]c4ccc(Cl)cc4c3CCN2C(=O)Oc2ccc(Cl)cc2)cc1] | 1159 |
| 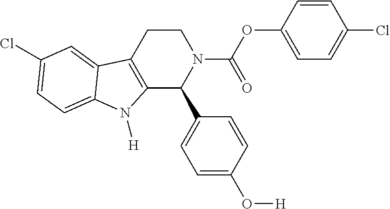 [SMILES: O=C(Oc1ccc(Cl)cc1)N1CCc2c([nH]c3ccc(Cl)cc23)[C@@H]1c1ccc(O)cc1] | 1160 |
| 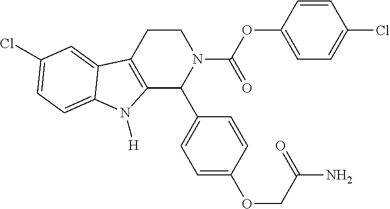 [SMILES: NC(=O)COc1ccc(C2c3[nH]c4ccc(Cl)cc4c3CCN2C(=O)Oc2ccc(Cl)cc2)cc1] | 1161 |
| 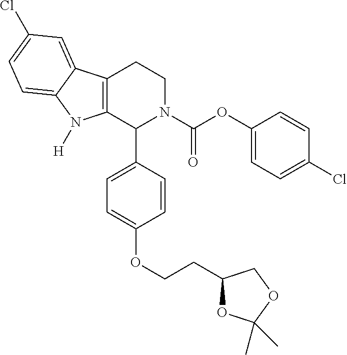 [SMILES: CC1(C)OC[C@H](CCOc2ccc(C3c4[nH]c5ccc(Cl)cc5c4CCN3C(=O)Oc3ccc(Cl)cc3)cc2)O1] | 1162 |
| 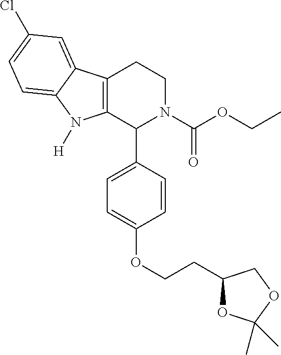 [SMILES: CCOC(=O)N1CCc2c([nH]c3ccc(Cl)cc23)C1c1ccc(OCC[C@H]2COC(C)(C)O2)cc1] | 1163 |
| 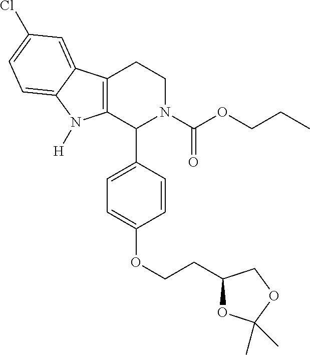 [SMILES: CCCOC(=O)N1CCc2c([nH]c3ccc(Cl)cc23)C1c1ccc(OCC[C@H]2COC(C)(C)O2)cc1] | 1164 |
| 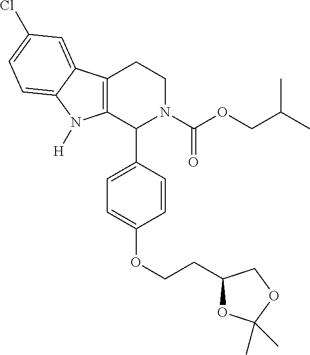 [SMILES: CC(C)COC(=O)N1CCc2c([nH]c3ccc(Cl)cc23)C1c1ccc(OCC[C@H]2COC(C)(C)O2)cc1] | 1165 |
| 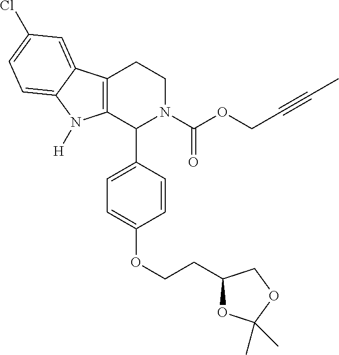 [SMILES: CC#CCOC(=O)N1CCc2c([nH]c3ccc(Cl)cc23)C1c1ccc(OCC[C@H]2COC(C)(C)O2)cc1] | 1166 |
| 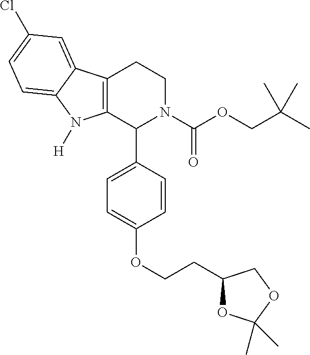 [SMILES: CC(C)(C)COC(=O)N1CCc2c([nH]c3ccc(Cl)cc23)C1c1ccc(OCC[C@H]2COC(C)(C)O2)cc1] | 1167 |
| 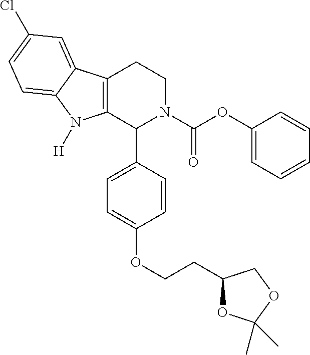 [SMILES: CC1(C)OC[C@H](CCOc2ccc(C3c4[nH]c5ccc(Cl)cc5c4CCN3C(=O)Oc3ccccc3)cc2)O1] | 1168 |
| 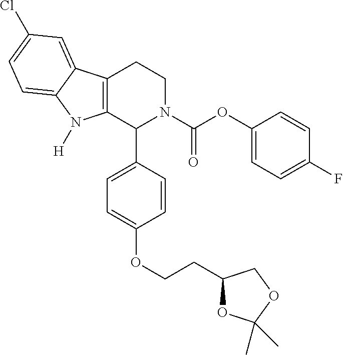 [SMILES: CC1(C)OC[C@H](CCOc2ccc(C3c4[nH]c5ccc(Cl)cc5c4CCN3C(=O)Oc3ccc(F)cc3)cc2)O1] | 1169 |
| 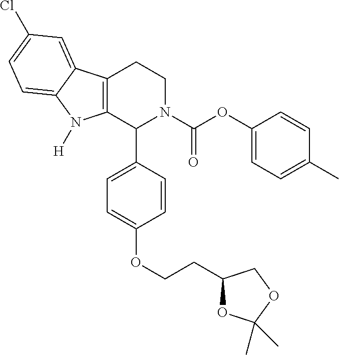 [SMILES: Cc1ccc(OC(=O)N2CCc3c([nH]c4ccc(Cl)cc34)C2c2ccc(OCC[C@H]3COC(C)(C)O3)cc2)cc1] | 1170 |
| 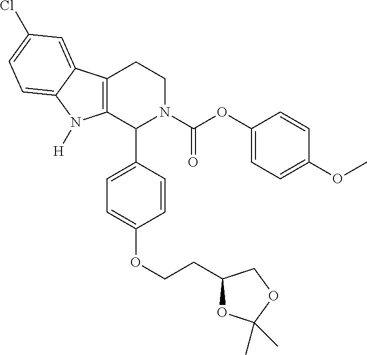 [SMILES: COc1ccc(OC(=O)N2CCc3c([nH]c4ccc(Cl)cc34)C2c2ccc(OCC[C@H]3COC(C)(C)O3)cc2)cc1] | 1171 |
| 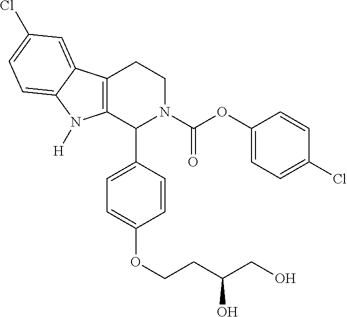 [SMILES: O=C(Oc1ccc(Cl)cc1)N1CCc2c([nH]c3ccc(Cl)cc23)C1c1ccc(OCC[C@H](O)CO)cc1] | 1172 |
| 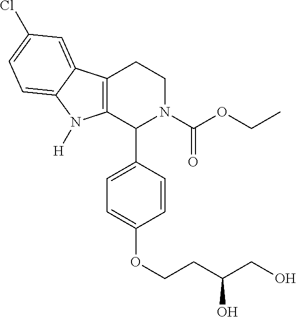 [SMILES: CCOC(=O)N1CCc2c([nH]c3ccc(Cl)cc23)C1c1ccc(OCC[C@H](O)CO)cc1] | 1173 |
| 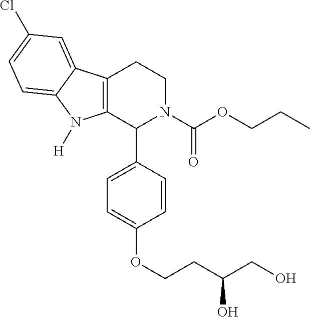 [SMILES: CCCOC(=O)N1CCc2c([nH]c3ccc(Cl)cc23)C1c1ccc(OCC[C@H](O)CO)cc1] | 1174 |
| 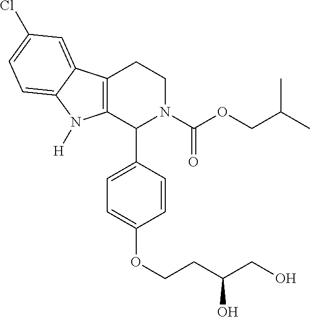 [SMILES: CC(C)COC(=O)N1CCc2c([nH]c3ccc(Cl)cc23)C1c1ccc(OCC[C@H](O)CO)cc1] | 1175 |
| 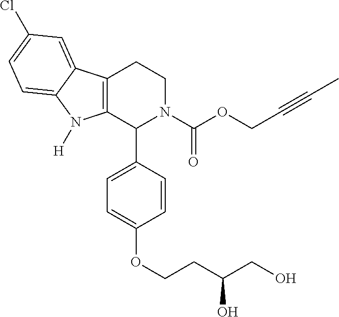 [SMILES: CC#CCOC(=O)N1CCc2c([nH]c3ccc(Cl)cc23)C1c1ccc(OCC[C@H](O)CO)cc1] | 1176 |
| 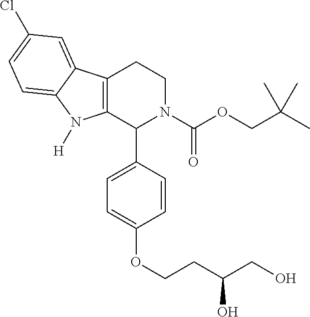 [SMILES: CC(C)(C)COC(=O)N1CCc2c([nH]c3ccc(Cl)cc23)C1c1ccc(OCC[C@H](O)CO)cc1] | 1177 |
| 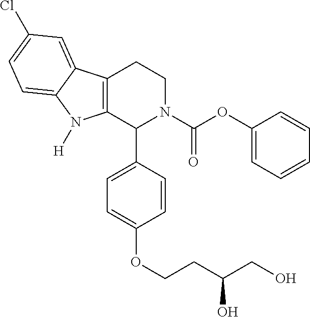 [SMILES: O=C(Oc1ccccc1)N1CCc2c([nH]c3ccc(Cl)cc23)C1c1ccc(OCC[C@H](O)CO)cc1] | 1178 |
| 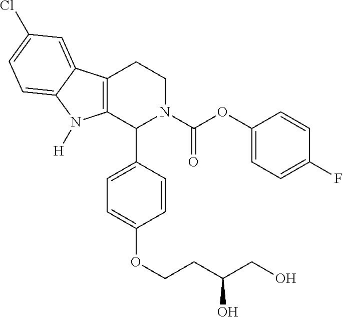 [SMILES: O=C(Oc1ccc(F)cc1)N1CCc2c([nH]c3ccc(Cl)cc23)C1c1ccc(OCC[C@H](O)CO)cc1] | 1179 |
| 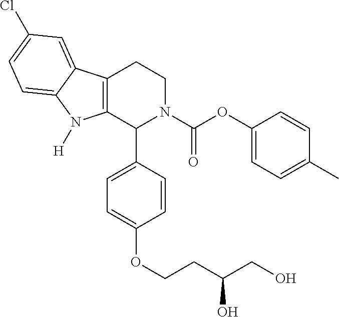 [SMILES: Cc1ccc(OC(=O)N2CCc3c([nH]c4ccc(Cl)cc34)C2c2ccc(OCC[C@H](O)CO)cc2)cc1] | 1180 |
| 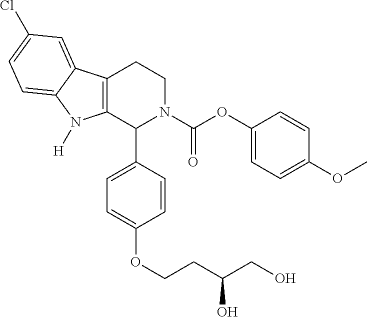 [SMILES: COc1ccc(OC(=O)N2CCc3c([nH]c4ccc(Cl)cc34)C2c2ccc(OCC[C@H](O)CO)cc2)cc1] | 1181 |
| 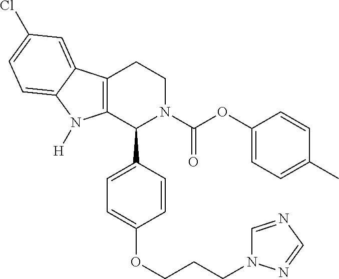 [SMILES: Cc1ccc(OC(=O)N2CCc3c([nH]c4ccc(Cl)cc34)[C@@H]2c2ccc(OCCCn3cncn3)cc2)cc1] | 1182 |
| 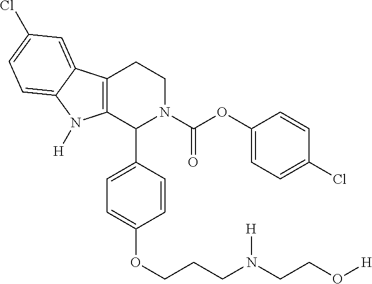 [SMILES: O=C(Oc1ccc(Cl)cc1)N1CCc2c([nH]c3ccc(Cl)cc23)C1c1ccc(OCCCNCCO)cc1] | 1183 |
| 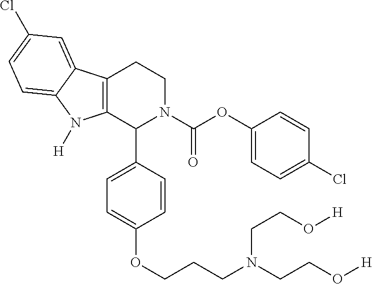 [SMILES: O=C(Oc1ccc(Cl)cc1)N1CCc2c([nH]c3ccc(Cl)cc23)C1c1ccc(OCCCN(CCO)CCO)cc1] | 1184 |
| 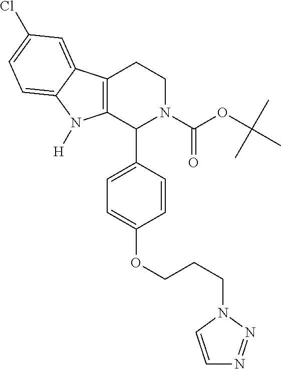 [SMILES: CC(C)(C)OC(=O)N1CCc2c([nH]c3ccc(Cl)cc23)C1c1ccc(OCCCn2ccnn2)cc1] | 1185 |
| 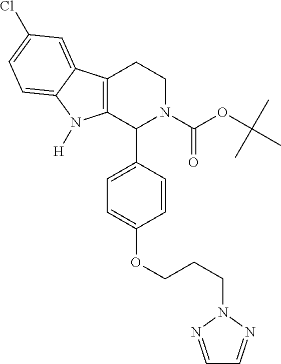 [SMILES: CC(C)(C)OC(=O)N1CCc2c([nH]c3ccc(Cl)cc23)C1c1ccc(OCCCn2nccn2)cc1] | 1186 |
| 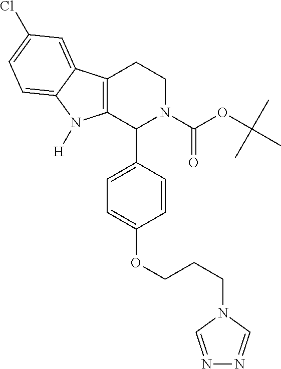 [SMILES: CC(C)(C)OC(=O)N1CCc2c([nH]c3ccc(Cl)cc23)C1c1ccc(OCCCn2cnnc2)cc1] | 1187 |
| 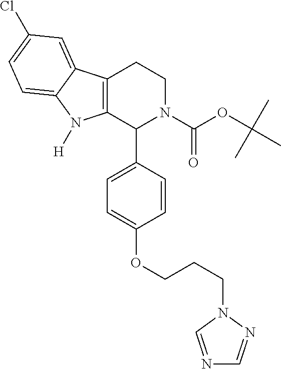 [SMILES: CC(C)(C)OC(=O)N1CCc2c([nH]c3ccc(Cl)cc23)C1c1ccc(OCCCn2cncn2)cc1] | 1188 |
| 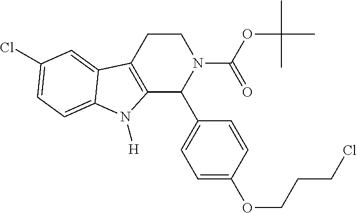 [SMILES: CC(C)(C)OC(=O)N1CCc2c([nH]c3ccc(Cl)cc23)C1c1ccc(OCCCCl)cc1] | 1189 |
| 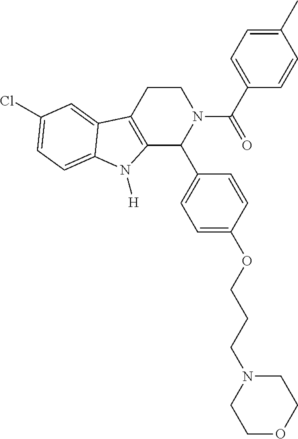 [SMILES: Cc1ccc(C(=O)N2CCc3c([nH]c4ccc(Cl)cc34)C2c2ccc(OCCCN3CCOCC3)cc2)cc1] | 1190 |
| 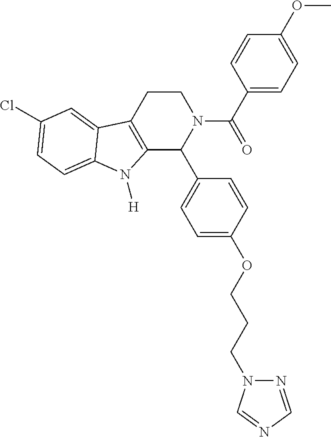 [SMILES: COc1ccc(C(=O)N2CCc3c([nH]c4ccc(Cl)cc34)C2c2ccc(OCCCn3cncn3)cc2)cc1] | 1191 |
| 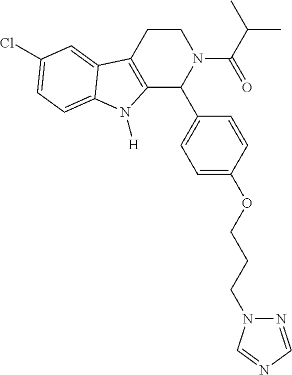 [SMILES: CC(C)C(=O)N1CCc2c([nH]c3ccc(Cl)cc23)C1c1ccc(OCCCn2cncn2)cc1] | 1192 |
| 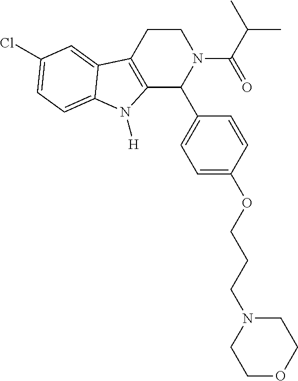 [SMILES: CC(C)C(=O)N1CCc2c([nH]c3ccc(Cl)cc23)C1c1ccc(OCCCN2CCOCC2)cc1] | 1193 |
| 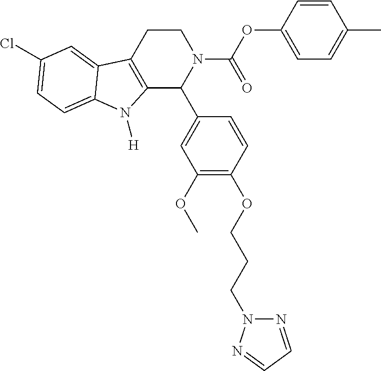 [SMILES: COc1cc(C2c3[nH]c4ccc(Cl)cc4c3CCN2C(=O)Oc2ccc(C)cc2)ccc1OCCCn1nccn1] | 1194 |
| 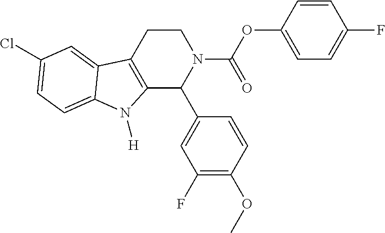 [SMILES: COc1ccc(C2c3[nH]c4ccc(Cl)cc4c3CCN2C(=O)Oc2ccc(F)cc2)cc1F] | 1195 |
| 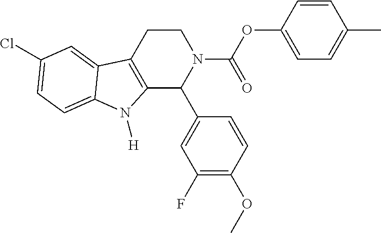 [SMILES: COc1ccc(C2c3[nH]c4ccc(Cl)cc4c3CCN2C(=O)Oc2ccc(C)cc2)cc1F] | 1196 |
| 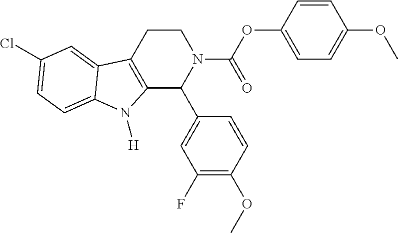 [SMILES: COc1ccc(OC(=O)N2CCc3c([nH]c4ccc(Cl)cc34)C2c2ccc(OC)c(F)c2)cc1] | 1197 |
| 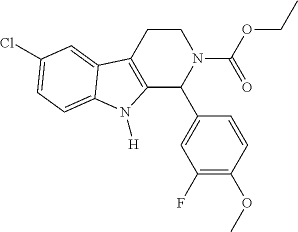 [SMILES: CCOC(=O)N1CCc2c([nH]c3ccc(Cl)cc23)C1c1ccc(OC)c(F)c1] | 1198 |
| 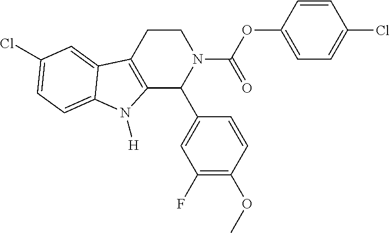 [SMILES: COc1ccc(C2c3[nH]c4ccc(Cl)cc4c3CCN2C(=O)Oc2ccc(Cl)cc2)cc1F] | 1199 |
| 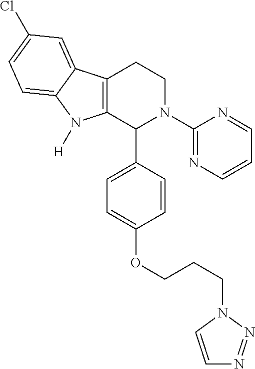 [SMILES: Clc1ccc2[nH]c3c(c2c1)CCN(c1ncccn1)C3c1ccc(OCCCn2ccnn2)cc1] | 1200 |
| 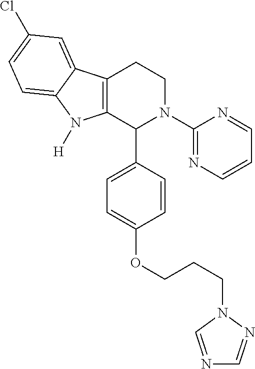 [SMILES: Clc1ccc2[nH]c3c(c2c1)CCN(c1ncccn1)C3c1ccc(OCCCn2cncn2)cc1] | 1201 |
| 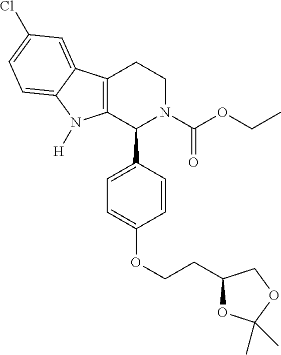 [SMILES: CCOC(=O)N1CCc2c([nH]c3ccc(Cl)cc23)[C@@H]1c1ccc(OCC[C@H]2COC(C)(C)O2)cc1] | 1202 |
| 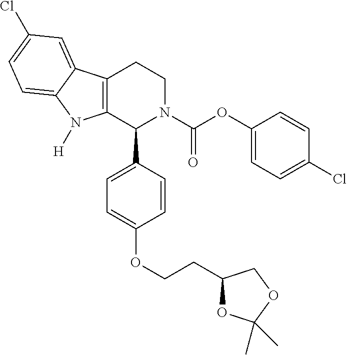 [SMILES: CC1(C)OC[C@H](CCOc2ccc([C@H]3c4[nH]c5ccc(Cl)cc5c4CCN3C(=O)Oc3ccc(Cl)cc3)cc2)O1] | 1203 |
| 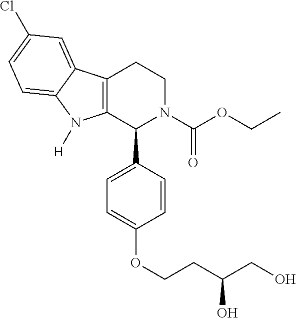 [SMILES: CCOC(=O)N1CCc2c([nH]c3ccc(Cl)cc23)[C@@H]1c1ccc(OCC[C@H](O)CO)cc1] | 1204 |
| 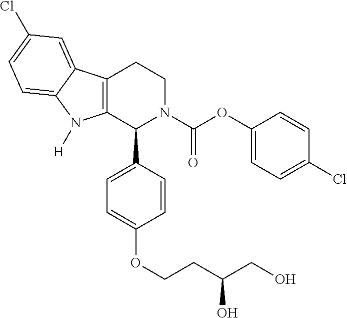 [SMILES: O=C(Oc1ccc(Cl)cc1)N1CCc2c([nH]c3ccc(Cl)cc23)[C@@H]1c1ccc(OCC[C@H](O)CO)cc1] | 1205 |
| 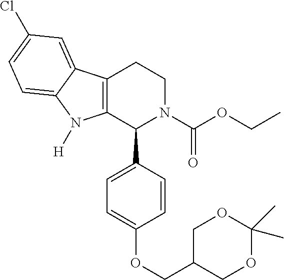 [SMILES: CCOC(=O)N1CCc2c([nH]c3ccc(Cl)cc23)[C@@H]1c1ccc(OCC2COC(C)(C)OC2)cc1] | 1206 |
| 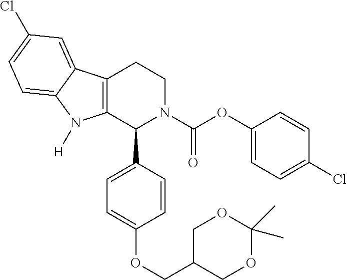 [SMILES: CC1(C)OCC(COc2ccc([C@H]3c4[nH]c5ccc(Cl)cc5c4CCN3C(=O)Oc3ccc(Cl)cc3)cc2)CO1] | 1207 |
| 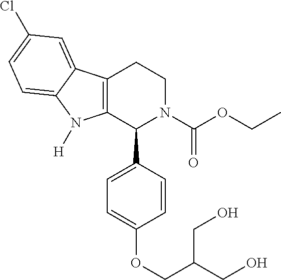 [SMILES: CCOC(=O)N1CCc2c([nH]c3ccc(Cl)cc23)[C@@H]1c1ccc(OCC(CO)CO)cc1] | 1208 |
| 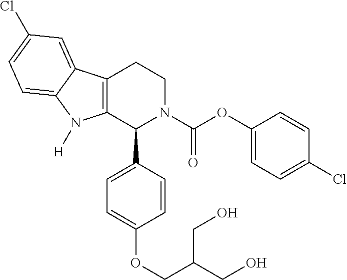 [SMILES: O=C(Oc1ccc(Cl)cc1)N1CCc2c([nH]c3ccc(Cl)cc23)[C@@H]1c1ccc(OCC(CO)CO)cc1] | 1209 |
| 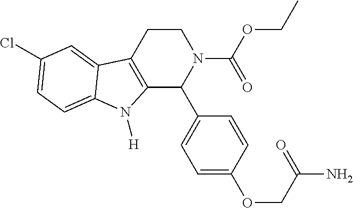 [SMILES: CCOC(=O)N1CCc2c([nH]c3ccc(Cl)cc23)C1c1ccc(OCC(N)=O)cc1] | 1210 |
| 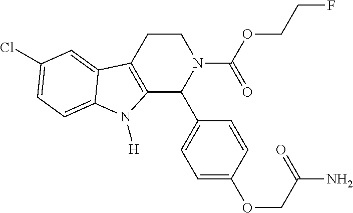 [SMILES: NC(=O)COc1ccc(C2c3[nH]c4ccc(Cl)cc4c3CCN2C(=O)OCCF)cc1] | 1211 |
| 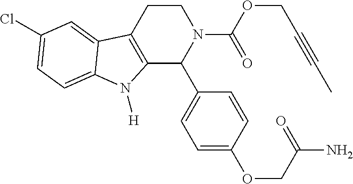 [SMILES: CC#CCOC(=O)N1CCc2c([nH]c3ccc(Cl)cc23)C1c1ccc(OCC(N)=O)cc1] | 1212 |
| 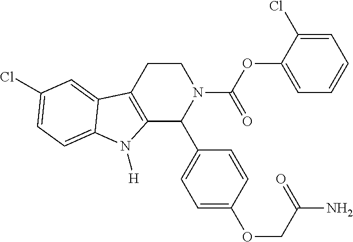 [SMILES: NC(=O)COc1ccc(C2c3[nH]c4ccc(Cl)cc4c3CCN2C(=O)Oc2ccccc2Cl)cc1] | 1213 |
| 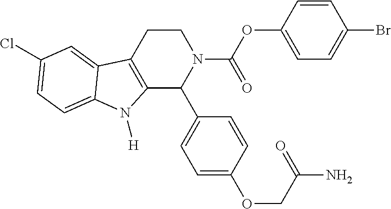 [SMILES: NC(=O)COc1ccc(C2c3[nH]c4ccc(Cl)cc4c3CCN2C(=O)Oc2ccc(Br)cc2)cc1] | 1214 |
| 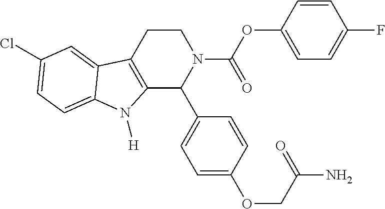 [SMILES: NC(=O)COc1ccc(C2c3[nH]c4ccc(Cl)cc4c3CCN2C(=O)Oc2ccc(F)cc2)cc1] | 1215 |
| 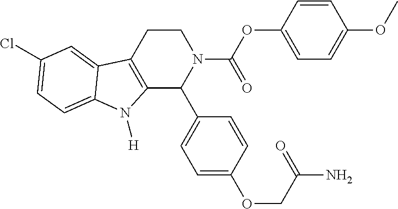 [SMILES: COc1ccc(OC(=O)N2CCc3c([nH]c4ccc(Cl)cc34)C2c2ccc(OCC(N)=O)cc2)cc1] | 1216 |
| 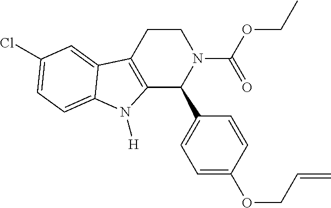 [SMILES: C=CCOc1ccc([C@H]2c3[nH]c4ccc(Cl)cc4c3CCN2C(=O)OCC)cc1] | 1217 |
| 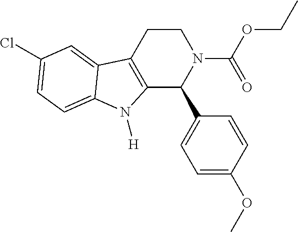 [SMILES: CCOC(=O)N1CCc2c([nH]c3ccc(Cl)cc23)[C@@H]1c1ccc(OC)cc1] | 1218 |
| 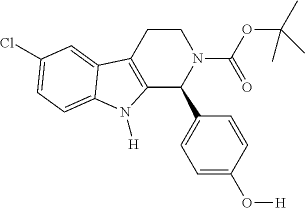 [SMILES: CC(C)(C)OC(=O)N1CCc2c([nH]c3ccc(Cl)cc23)[C@@H]1c1ccc(O)cc1] | 1219 |
| 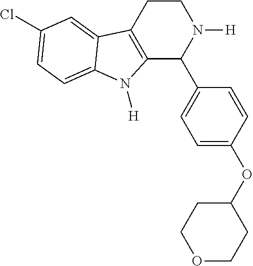 [SMILES: Clc1ccc2[nH]c3c(c2c1)CCNC3c1ccc(OC2CCOCC2)cc1] | 1220 |
| 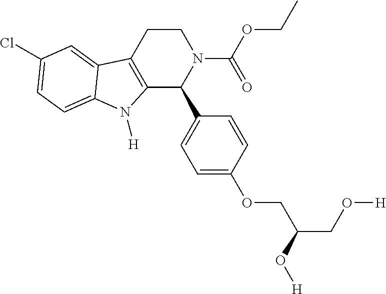 [SMILES: CCOC(=O)N1CCc2c([nH]c3ccc(Cl)cc23)[C@@H]1c1ccc(OC[C@H](O)CO)cc1] | 1221 |
| 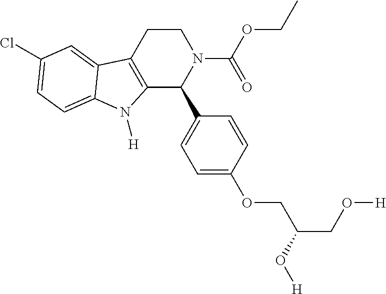 [SMILES: CCOC(=O)N1CCc2c([nH]c3ccc(Cl)cc23)[C@@H]1c1ccc(OC[C@@H](O)CO)cc1] | 1222 |
| 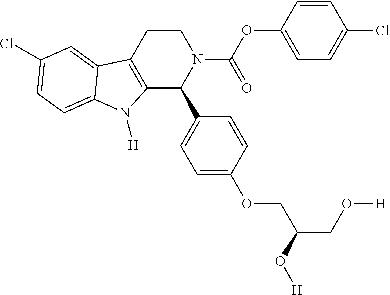 [SMILES: O=C(Oc1ccc(Cl)cc1)N1CCc2c([nH]c3ccc(Cl)cc23)[C@@H]1c1ccc(OC[C@H](O)CO)cc1] | 1223 |
| 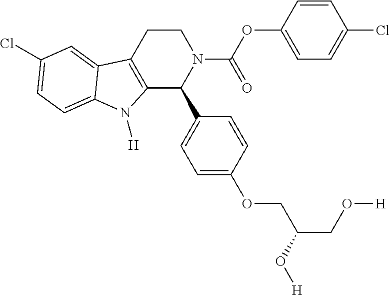 [SMILES: O=C(Oc1ccc(Cl)cc1)N1CCc2c([nH]c3ccc(Cl)cc23)[C@@H]1c1ccc(OC[C@@H](O)CO)cc1] | 1224 |
| 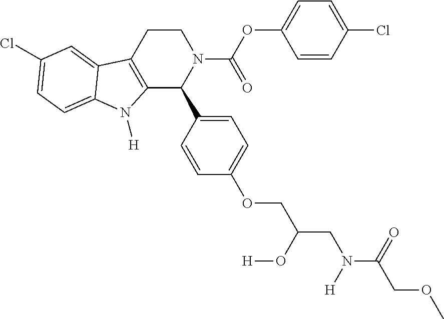 [SMILES: COCC(=O)NCC(O)COc1ccc([C@H]2c3[nH]c4ccc(Cl)cc4c3CCN2C(=O)Oc2ccc(Cl)cc2)cc1] | 1225 |
| 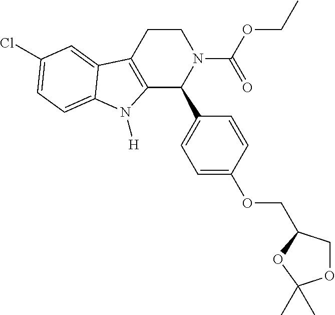 [SMILES: CCOC(=O)N1CCc2c([nH]c3ccc(Cl)cc23)[C@@H]1c1ccc(OC[C@H]2COC(C)(C)O2)cc1] | 1226 |
| 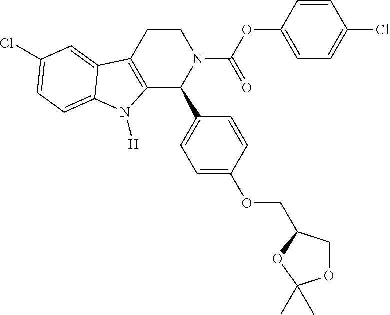 [SMILES: CC1(C)OC[C@H](COc2ccc([C@H]3c4[nH]c5ccc(Cl)cc5c4CCN3C(=O)Oc3ccc(Cl)cc3)cc2)O1] | 1227 |
| 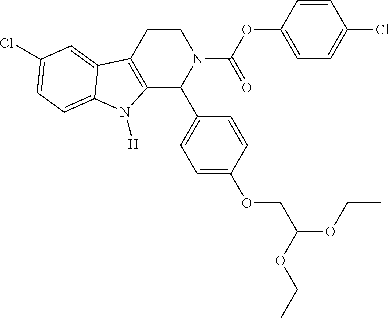 [SMILES: CCOC(COc1ccc(C2c3[nH]c4ccc(Cl)cc4c3CCN2C(=O)Oc2ccc(Cl)cc2)cc1)OCC] | 1228 |
| 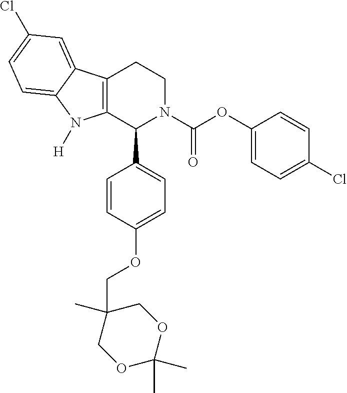 [SMILES: CC1(COc2ccc([C@H]3c4[nH]c5ccc(Cl)cc5c4CCN3C(=O)Oc3ccc(Cl)cc3)cc2)COC(C)(C)OC1] | 1229 |
| 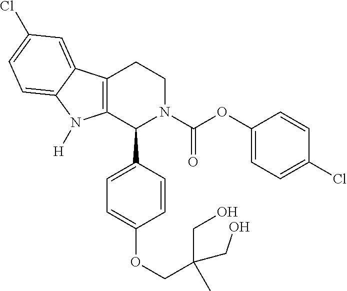 [SMILES: CC(CO)(CO)COc1ccc([C@H]2c3[nH]c4ccc(Cl)cc4c3CCN2C(=O)Oc2ccc(Cl)cc2)cc1] | 1230 |
| 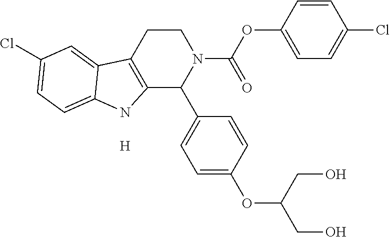 | 1231 |
| 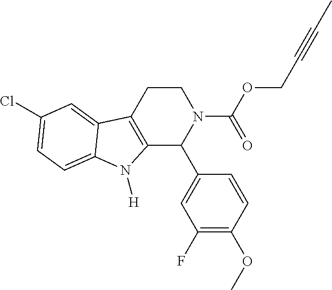 [SMILES: CC#CCOC(=O)N1CCc2c([nH]c3ccc(Cl)cc23)C1c1ccc(OC)c(F)c1] | 1232 |
| 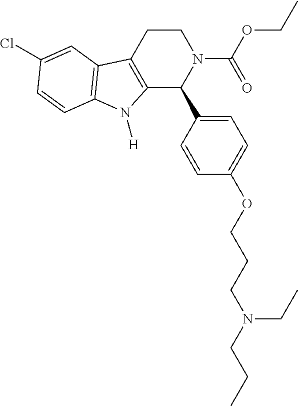 [SMILES: CCCN(CC)CCCOc1ccc([C@H]2c3[nH]c4ccc(Cl)cc4c3CCN2C(=O)OCC)cc1] | 1233 |
| 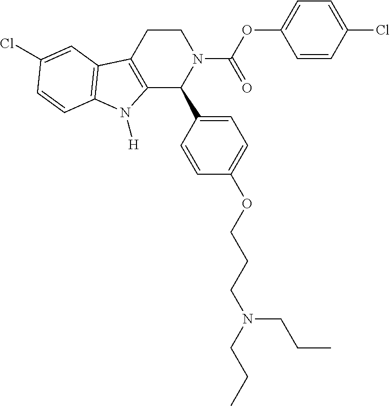 [SMILES: CCCN(CCC)CCCOc1ccc([C@H]2c3[nH]c4ccc(Cl)cc4c3CCN2C(=O)Oc2ccc(Cl)cc2)cc1] | 1234 |
| 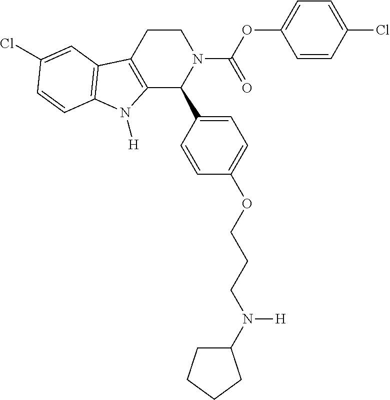 [SMILES: O=C(Oc1ccc(Cl)cc1)N1CCc2c([nH]c3ccc(Cl)cc23)[C@@H]1c1ccc(OCCCNC2CCCC2)cc1] | 1235 |
| 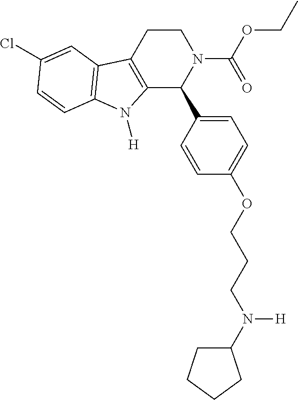 [SMILES: CCOC(=O)N1CCc2c([nH]c3ccc(Cl)cc23)[C@@H]1c1ccc(OCCCNC2CCCC2)cc1] | 1236 |
| 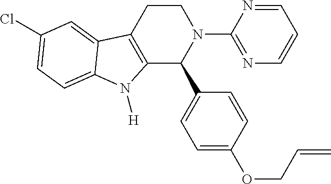 [SMILES: C=CCOc1ccc([C@H]2c3[nH]c4ccc(Cl)cc4c3CCN2c2ncccn2)cc1] | 1237 |
| 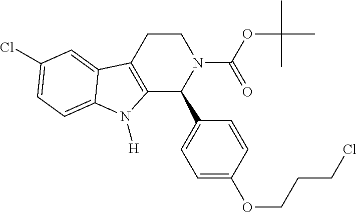 [SMILES: CC(C)(C)OC(=O)N1CCc2c([nH]c3ccc(Cl)cc23)[C@@H]1c1ccc(OCCCCl)cc1] | 1238 |
| 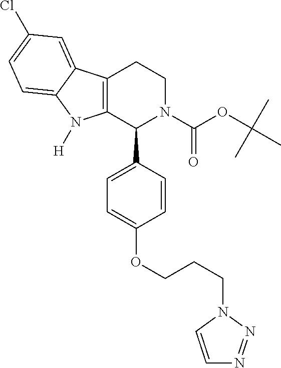 [SMILES: CC(C)(C)OC(=O)N1CCc2c([nH]c3ccc(Cl)cc23)[C@@H]1c1ccc(OCCCn2ccnn2)cc1] | 1239 |
| 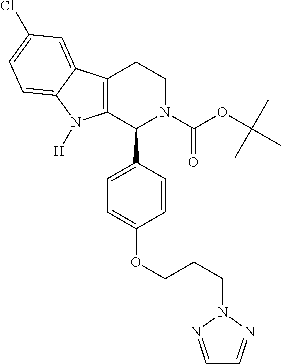 [SMILES: CC(C)(C)OC(=O)N1CCc2c([nH]c3ccc(Cl)cc23)[C@@H]1c1ccc(OCCCn2nccn2)cc1] | 1240 |
| 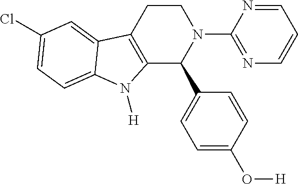 [SMILES: Oc1ccc([C@H]2c3[nH]c4ccc(Cl)cc4c3CCN2c2ncccn2)cc1] | 1241 |
| 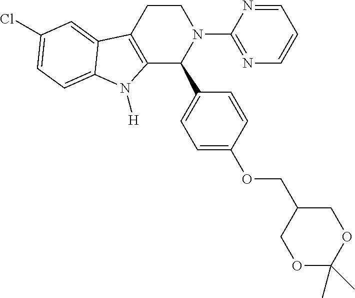 [SMILES: CC1(C)OCC(COc2ccc([C@H]3c4[nH]c5ccc(Cl)cc5c4CCN3c3ncccn3)cc2)CO1] | 1242 |
| 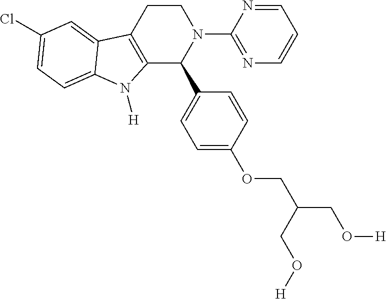 [SMILES: OCC(CO)COc1ccc([C@H]2c3[nH]c4ccc(Cl)cc4c3CCN2c2ncccn2)cc1] | 1243 |
| 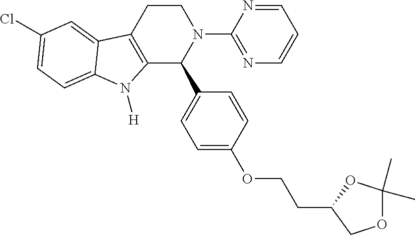 [SMILES: CC1(C)OC[C@H](CCOc2ccc([C@H]3c4[nH]c5ccc(Cl)cc5c4CCN3c3ncccn3)cc2)O1] | 1244 |
| 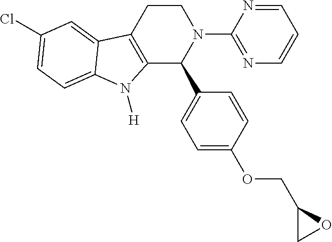 [SMILES: Clc1ccc2[nH]c3c(c2c1)CCN(c1ncccn1)[C@H]3c1ccc(OC[C@@H]2CO2)cc1] | 1245 |
| 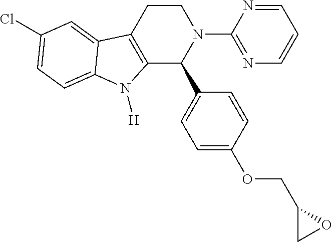 [SMILES: Clc1ccc2[nH]c3c(c2c1)CCN(c1ncccn1)[C@H]3c1ccc(OC[C@H]2CO2)cc1] | 1246 |
| 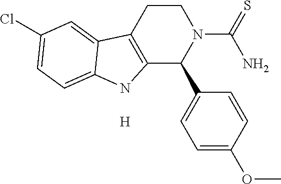 | 1247 |
| 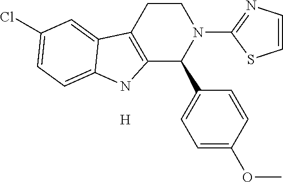 | 1248 |
| 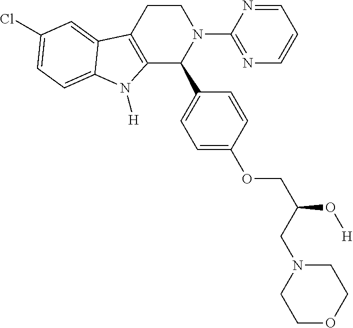 [SMILES: O[C@H](COc1ccc([C@H]2c3[nH]c4ccc(Cl)cc4c3CCN2c2ncccn2)cc1)CN1CCOCC1] | 1249 |
| 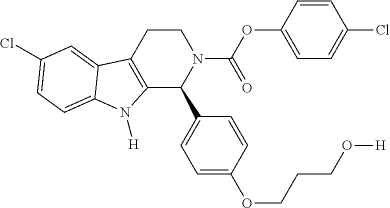 [SMILES: O=C(Oc1ccc(Cl)cc1)N1CCc2c([nH]c3ccc(Cl)cc23)[C@@H]1c1ccc(OCCCO)cc1] | 1250 |
| 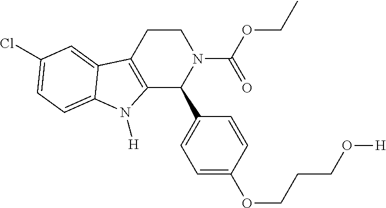 [SMILES: CCOC(=O)N1CCc2c([nH]c3ccc(Cl)cc23)[C@@H]1c1ccc(OCCCO)cc1] | 1251 |
| 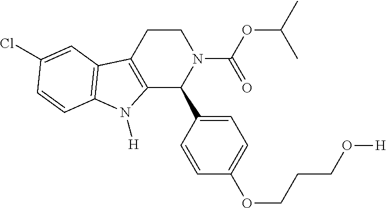 [SMILES: CC(C)OC(=O)N1CCc2c([nH]c3ccc(Cl)cc23)[C@@H]1c1ccc(OCCCO)cc1] | 1252 |
| 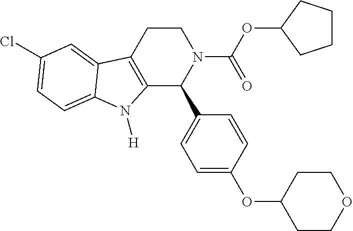 [SMILES: O=C(OC1CCCC1)N1CCc2c([nH]c3ccc(Cl)cc23)[C@@H]1c1ccc(OC2CCOCC2)cc1] | 1253 |
| 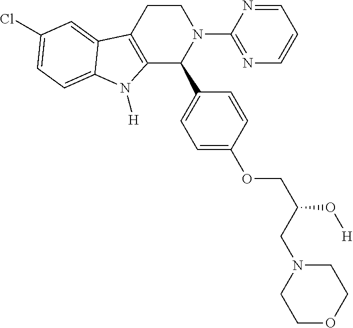 [SMILES: O[C@@H](COc1ccc([C@H]2c3[nH]c4ccc(Cl)cc4c3CCN2c2ncccn2)cc1)CN1CCOCC1] | 1254 |
| 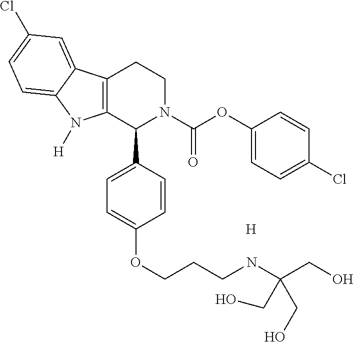 | 1255 |
| 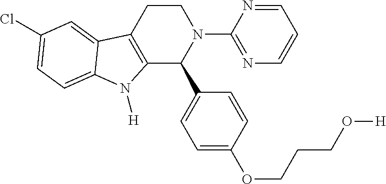 [SMILES: OCCCOc1ccc([C@H]2c3[nH]c4ccc(Cl)cc4c3CCN2c2ncccn2)cc1] | 1256 |
| 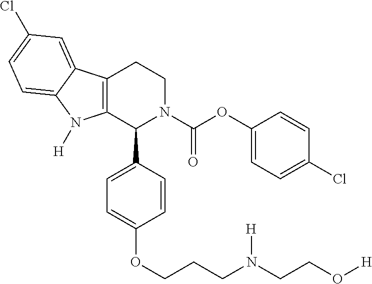 [SMILES: O=C(Oc1ccc(Cl)cc1)N1CCc2c([nH]c3ccc(Cl)cc23)[C@@H]1c1ccc(OCCCNCCO)cc1] | 1257 |
| 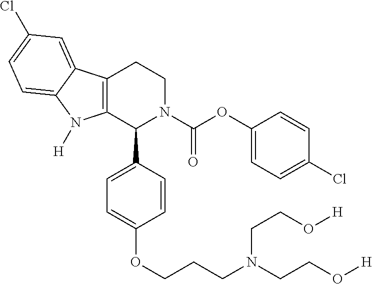 [SMILES: O=C(Oc1ccc(Cl)cc1)N1CCc2c([nH]c3ccc(Cl)cc23)[C@@H]1c1ccc(OCCCN(CCO)CCO)cc1] | 1258 |
| 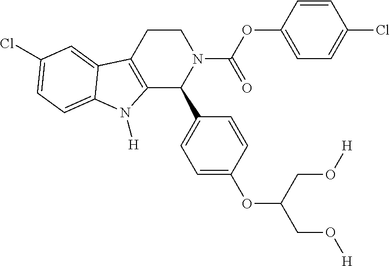 [SMILES: O=C(Oc1ccc(Cl)cc1)N1CCc2c([nH]c3ccc(Cl)cc23)[C@@H]1c1ccc(OC(CO)CO)cc1] | 1259 |
| 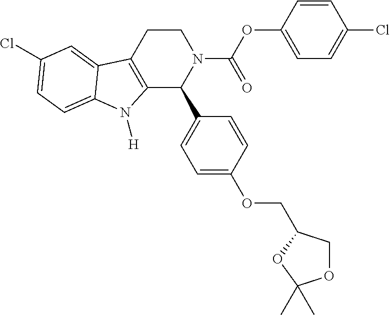 [SMILES: CC1(C)OC[C@@H](COc2ccc([C@H]3c4[nH]c5ccc(Cl)cc5c4CCN3C(=O)Oc3ccc(Cl)cc3)cc2)O1] | 1260 |
| 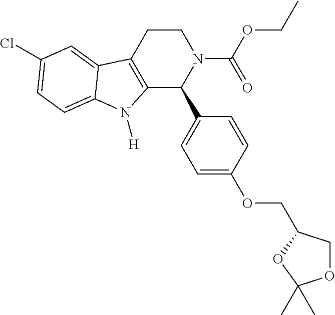 [SMILES: CCOC(=O)N1CCc2c([nH]c3ccc(Cl)cc23)[C@@H]1c1ccc(OC[C@@H]2COC(C)(C)O2)cc1] | 1261 |
| 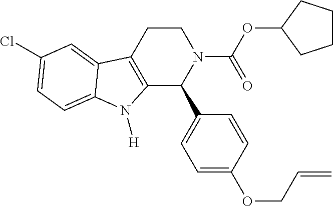 [SMILES: C=CCOc1ccc([C@H]2c3[nH]c4ccc(Cl)cc4c3CCN2C(=O)OC2CCCC2)cc1] | 1262 |
| 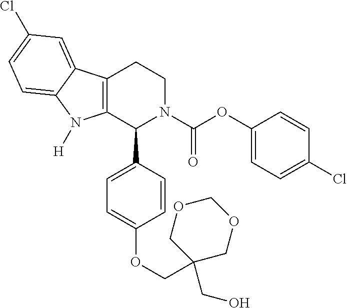 [SMILES: O=C(Oc1ccc(Cl)cc1)N1CCc2c([nH]c3ccc(Cl)cc23)[C@@H]1c1ccc(OCC2(CO)COCOC2)cc1] | 1263 |
| 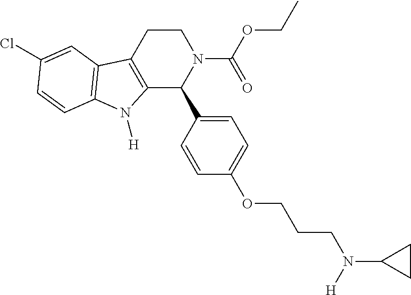 [SMILES: CCOC(=O)N1CCc2c([nH]c3ccc(Cl)cc23)[C@@H]1c1ccc(OCCCNC2CC2)cc1] | 1264 |
| 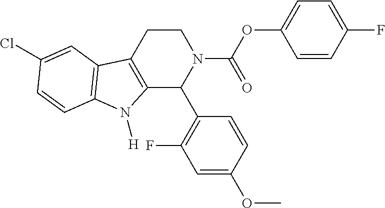 [SMILES: COc1ccc(C2c3[nH]c4ccc(Cl)cc4c3CCN2C(=O)Oc2ccc(F)cc2)c(F)c1] | 1265 |
| 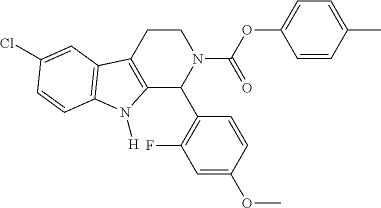 [SMILES: COc1ccc(C2c3[nH]c4ccc(Cl)cc4c3CCN2C(=O)Oc2ccc(C)cc2)c(F)c1] | 1266 |
| 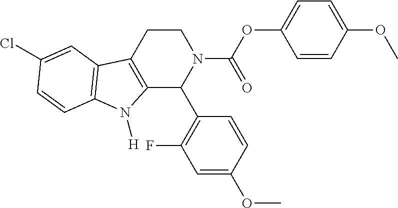 [SMILES: COc1ccc(OC(=O)N2CCc3c([nH]c4ccc(Cl)cc34)C2c2ccc(OC)cc2F)cc1] | 1267 |
| 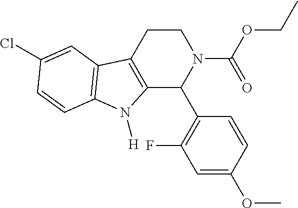 [SMILES: CCOC(=O)N1CCc2c([nH]c3ccc(Cl)cc23)C1c1ccc(OC)cc1F] | 1268 |
| 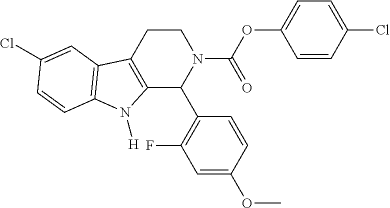 [SMILES: COc1ccc(C2c3[nH]c4ccc(Cl)cc4c3CCN2C(=O)Oc2ccc(Cl)cc2)c(F)c1] | 1269 |
| 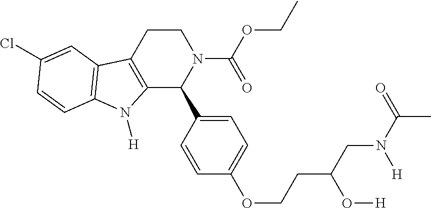 [SMILES: CCOC(=O)N1CCc2c([nH]c3ccc(Cl)cc23)[C@@H]1c1ccc(OCCC(O)CNC(C)=O)cc1] | 1270 |
| 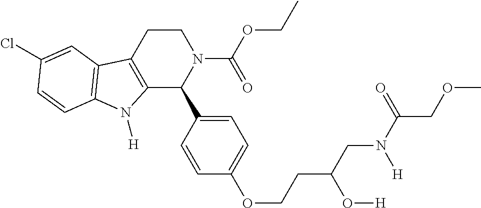 [SMILES: CCOC(=O)N1CCc2c([nH]c3ccc(Cl)cc23)[C@@H]1c1ccc(OCCC(O)CNC(=O)COC)cc1] | 1271 |
| 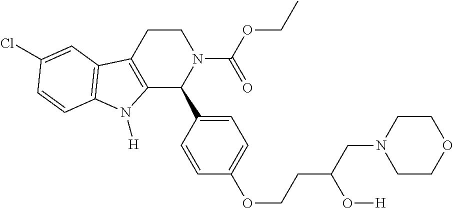 [SMILES: CCOC(=O)N1CCc2c([nH]c3ccc(Cl)cc23)[C@@H]1c1ccc(OCCC(O)CN2CCOCC2)cc1] | 1272 |
| 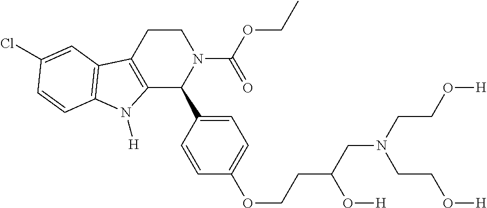 [SMILES: CCOC(=O)N1CCc2c([nH]c3ccc(Cl)cc23)[C@@H]1c1ccc(OCCC(O)CN(CCO)CCO)cc1] | 1273 |
| 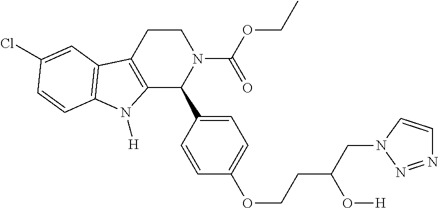 [SMILES: CCOC(=O)N1CCc2c([nH]c3ccc(Cl)cc23)[C@@H]1c1ccc(OCCC(O)Cn2ccnn2)cc1] | 1274 |
| 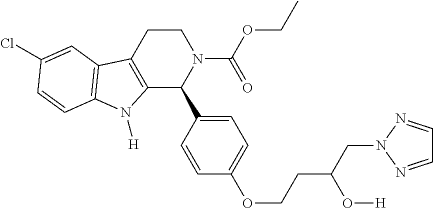 [SMILES: CCOC(=O)N1CCc2c([nH]c3ccc(Cl)cc23)[C@@H]1c1ccc(OCCC(O)Cn2nccn2)cc1] | 1275 |
| 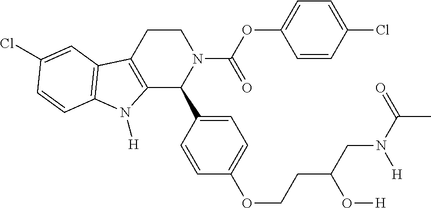 [SMILES: CC(=O)NCC(O)CCOc1ccc([C@H]2c3[nH]c4ccc(Cl)cc4c3CCN2C(=O)Oc2ccc(Cl)cc2)cc1] | 1276 |
| 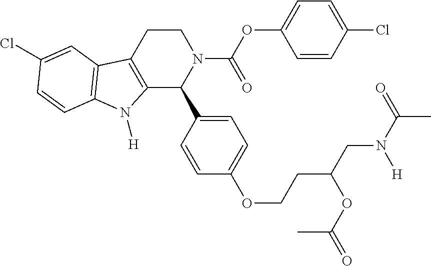 [SMILES: CC(=O)NCC(CCOc1ccc([C@H]2c3[nH]c4ccc(Cl)cc4c3CCN2C(=O)Oc2ccc(Cl)cc2)cc1)OC(C)=O] | 1277 |
| 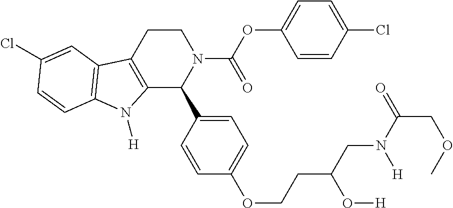 [SMILES: COCC(=O)NCC(O)CCOc1ccc([C@H]2c3[nH]c4ccc(Cl)cc4c3CCN2C(=O)Oc2ccc(Cl)cc2)cc1] | 1278 |
| 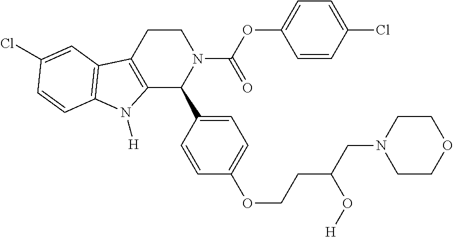 [SMILES: O=C(Oc1ccc(Cl)cc1)N1CCc2c([nH]c3ccc(Cl)cc23)[C@@H]1c1ccc(OCCC(O)CN2CCOCC2)cc1] | 1279 |
| 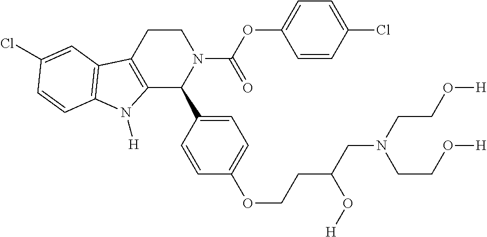 [SMILES: O=C(Oc1ccc(Cl)cc1)N1CCc2c([nH]c3ccc(Cl)cc23)[C@@H]1c1ccc(OCCC(O)CN(CCO)CCO)cc1] | 1280 |
| 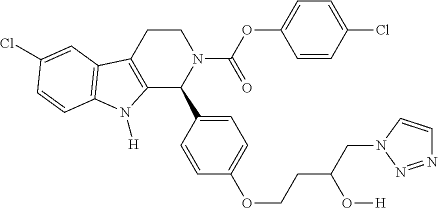 [SMILES: O=C(Oc1ccc(Cl)cc1)N1CCc2c([nH]c3ccc(Cl)cc23)[C@@H]1c1ccc(OCCC(O)Cn2ccnn2)cc1] | 1281 |
| 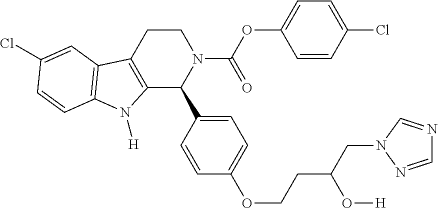 [SMILES: O=C(Oc1ccc(Cl)cc1)N1CCc2c([nH]c3ccc(Cl)cc23)[C@@H]1c1ccc(OCCC(O)Cn2cncn2)cc1] | 1282 |
| 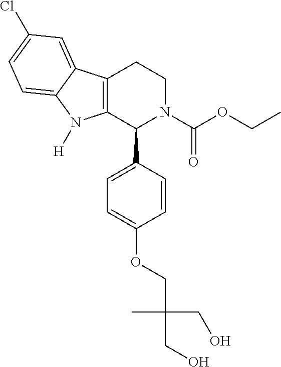 [SMILES: CCOC(=O)N1CCc2c([nH]c3ccc(Cl)cc23)[C@@H]1c1ccc(OCC(C)(CO)CO)cc1] | 1283 |
| 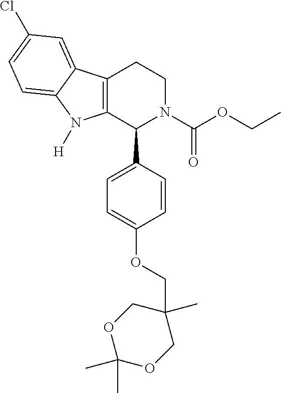 [SMILES: CCOC(=O)N1CCc2c([nH]c3ccc(Cl)cc23)[C@@H]1c1ccc(OCC2(C)COC(C)(C)OC2)cc1] | 1284 |
| 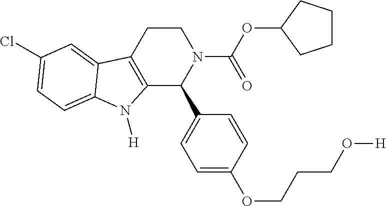 [SMILES: O=C(OC1CCCC1)N1CCc2c([nH]c3ccc(Cl)cc23)[C@@H]1c1ccc(OCCCO)cc1] | 1285 |
| 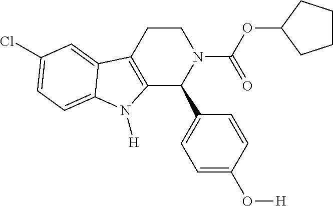 [SMILES: O=C(OC1CCCC1)N1CCc2c([nH]c3ccc(Cl)cc23)[C@@H]1c1ccc(O)cc1] | 1286 |
| 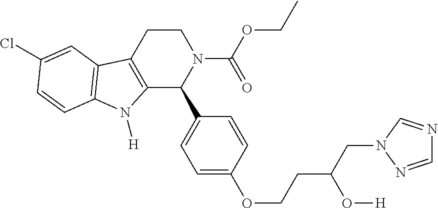 [SMILES: CCOC(=O)N1CCc2c([nH]c3ccc(Cl)cc23)[C@@H]1c1ccc(OCCC(O)Cn2cncn2)cc1] | 1287 |
| 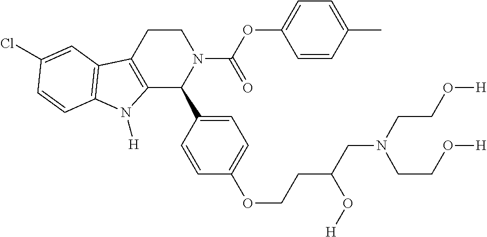 [SMILES: Cc1ccc(OC(=O)N2CCc3c([nH]c4ccc(Cl)cc34)[C@@H]2c2ccc(OCCC(O)CN(CCO)CCO)cc2)cc1] | 1288 |
| 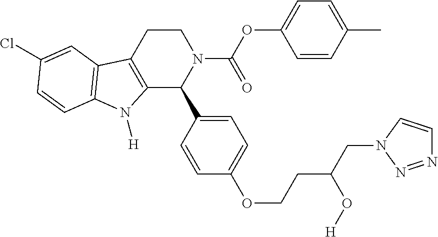 [SMILES: Cc1ccc(OC(=O)N2CCc3c([nH]c4ccc(Cl)cc34)[C@@H]2c2ccc(OCCC(O)Cn3ccnn3)cc2)cc1] | 1289 |
| 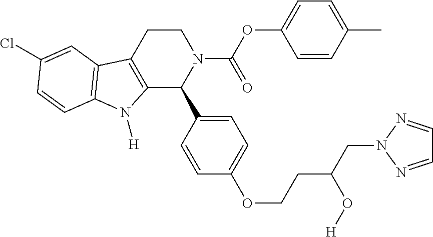 [SMILES: Cc1ccc(OC(=O)N2CCc3c([nH]c4ccc(Cl)cc34)[C@@H]2c2ccc(OCCC(O)Cn3nccn3)cc2)cc1] | 1290 |
| 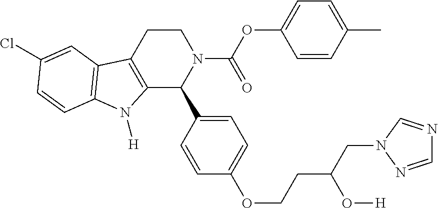 [SMILES: Cc1ccc(OC(=O)N2CCc3c([nH]c4ccc(Cl)cc34)[C@@H]2c2ccc(OCCC(O)Cn3cncn3)cc2)cc1] | 1291 |
| 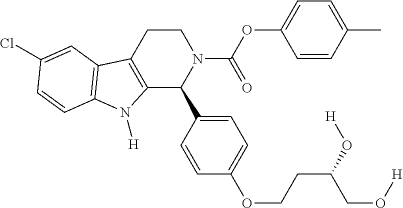 [SMILES: Cc1ccc(OC(=O)N2CCc3c([nH]c4ccc(Cl)cc34)[C@@H]2c2ccc(OCC[C@H](O)CO)cc2)cc1] | 1292 |
| 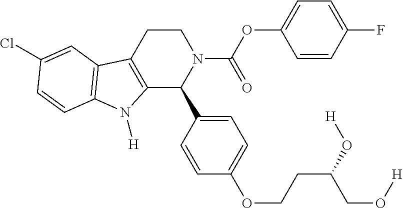 [SMILES: O=C(Oc1ccc(F)cc1)N1CCc2c([nH]c3ccc(Cl)cc23)[C@@H]1c1ccc(OCC[C@H](O)CO)cc1] | 1293 |
| 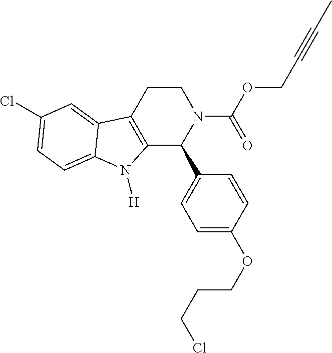 [SMILES: CC#CCOC(=O)N1CCc2c([nH]c3ccc(Cl)cc23)[C@@H]1c1ccc(OCCCCl)cc1] | 1294 |
| 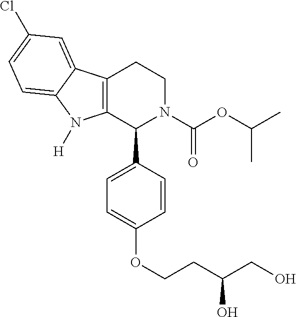 [SMILES: CC(C)OC(=O)N1CCc2c([nH]c3ccc(Cl)cc23)[C@@H]1c1ccc(OCC[C@H](O)CO)cc1] | 1295 |
| 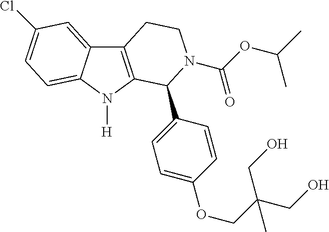 [SMILES: CC(C)OC(=O)N1CCc2c([nH]c3ccc(Cl)cc23)[C@@H]1c1ccc(OCC(C)(CO)CO)cc1] | 1296 |
| 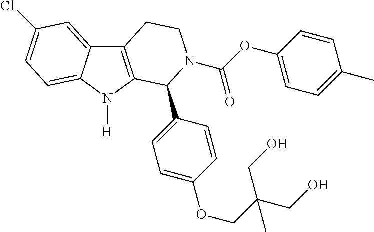 [SMILES: Cc1ccc(OC(=O)N2CCc3c([nH]c4ccc(Cl)cc34)[C@@H]2c2ccc(OCC(C)(CO)CO)cc2)cc1] | 1297 |
| 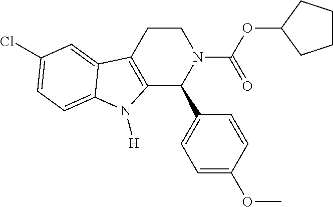 [SMILES: COc1ccc([C@H]2c3[nH]c4ccc(Cl)cc4c3CCN2C(=O)OC2CCCC2)cc1] | 1298 |
| 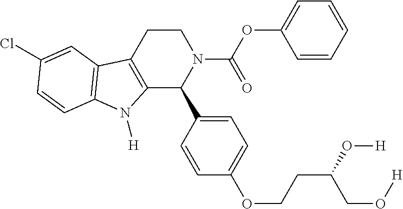 [SMILES: O=C(Oc1ccccc1)N1CCc2c([nH]c3ccc(Cl)cc23)[C@@H]1c1ccc(OCC[C@H](O)CO)cc1] | 1299 |
| 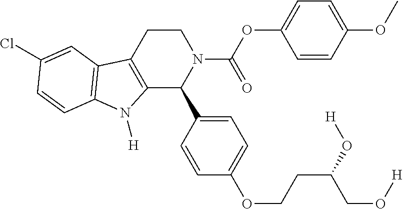 [SMILES: COc1ccc(OC(=O)N2CCc3c([nH]c4ccc(Cl)cc34)[C@@H]2c2ccc(OCC[C@H](O)CO)cc2)cc1] | 1300 |
| 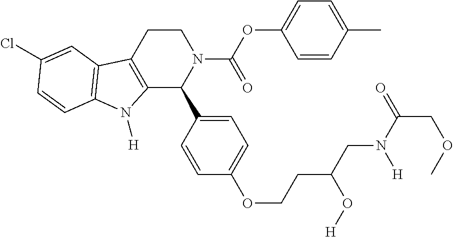 [SMILES: COCC(=O)NCC(O)CCOc1ccc([C@H]2c3[nH]c4ccc(Cl)cc4c3CCN2C(=O)Oc2ccc(C)cc2)cc1] | 1301 |
| 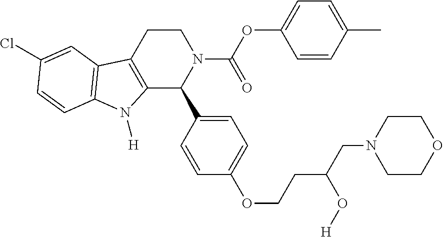 [SMILES: Cc1ccc(OC(=O)N2CCc3c([nH]c4ccc(Cl)cc34)[C@@H]2c2ccc(OCCC(O)CN3CCOCC3)cc2)cc1] | 1302 |
| 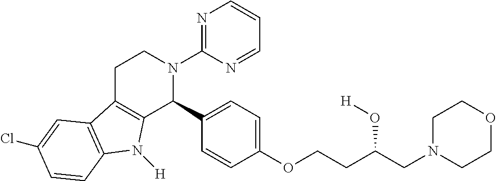 [SMILES: O[C@@H](CCOc1ccc([C@H]2c3[nH]c4ccc(Cl)cc4c3CCN2c2ncccn2)cc1)CN1CCOCC1] | 1303 |
| 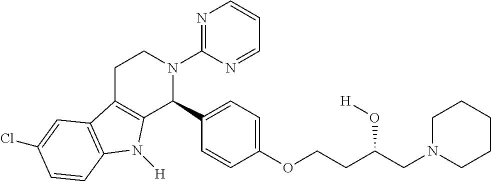 [SMILES: O[C@@H](CCOc1ccc([C@H]2c3[nH]c4ccc(Cl)cc4c3CCN2c2ncccn2)cc1)CN1CCCCC1] | 1304 |
| 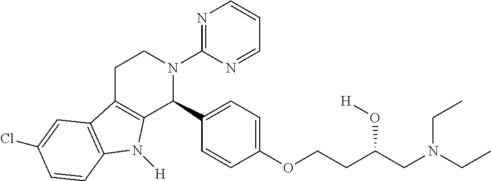 [SMILES: CCN(CC)C[C@@H](O)CCOc1ccc([C@H]2c3[nH]c4ccc(Cl)cc4c3CCN2c2ncccn2)cc1] | 1305 |
| 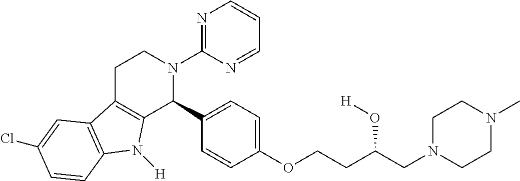 [SMILES: CN1CCN(C[C@@H](O)CCOc2ccc([C@H]3c4[nH]c5ccc(Cl)cc5c4CCN3c3ncccn3)cc2)CC1] | 1306 |
| 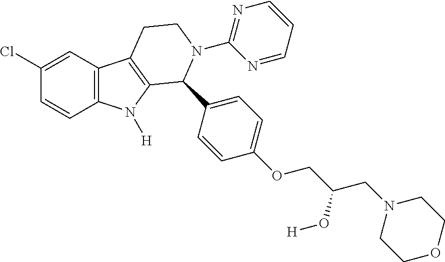 [SMILES: O[C@H](COc1ccc([C@H]2c3[nH]c4ccc(Cl)cc4c3CCN2c2ncccn2)cc1)CN1CCOCC1] | 1307 |
| 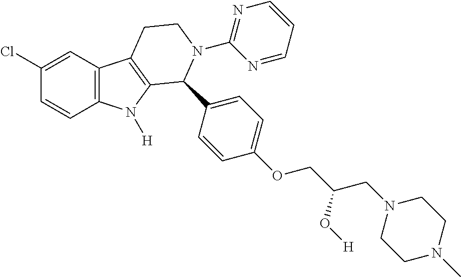 [SMILES: CN1CCN(C[C@H](O)COc2ccc([C@H]3c4[nH]c5ccc(Cl)cc5c4CCN3c3ncccn3)cc2)CC1] | 1308 |
| 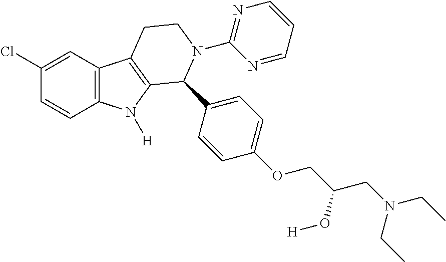 [SMILES: CCN(CC)C[C@H](O)COc1ccc([C@H]2c3[nH]c4ccc(Cl)cc4c3CCN2c2ncccn2)cc1] | 1309 |
| 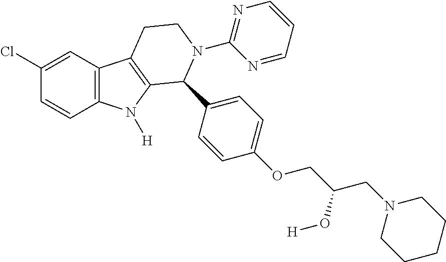 [SMILES: O[C@H](COc1ccc([C@H]2c3[nH]c4ccc(Cl)cc4c3CCN2c2ncccn2)cc1)CN1CCCCC1] | 1310 |
| 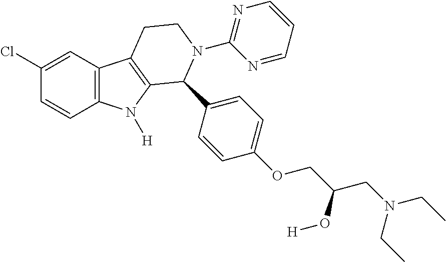 [SMILES: CCN(CC)C[C@@H](O)COc1ccc([C@H]2c3[nH]c4ccc(Cl)cc4c3CCN2c2ncccn2)cc1] | 1311 |
| 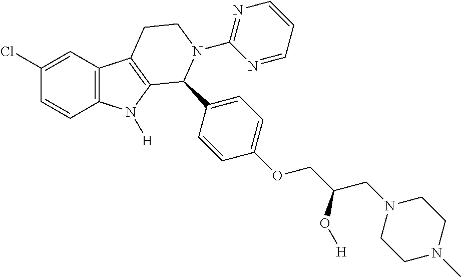 [SMILES: CN1CCN(C[C@@H](O)COc2ccc([C@H]3c4[nH]c5ccc(Cl)cc5c4CCN3c3ncccn3)cc2)CC1] | 1312 |
| 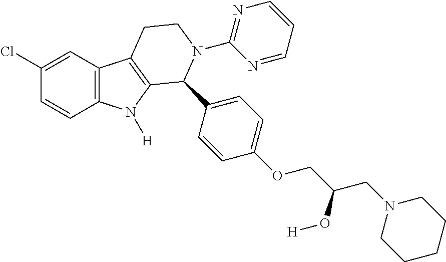 [SMILES: O[C@@H](COc1ccc([C@H]2c3[nH]c4ccc(Cl)cc4c3CCN2c2ncccn2)cc1)CN1CCCCC1] | 1313 |
| 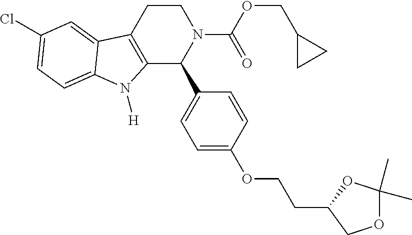 [SMILES: CC1(C)OC[C@H](CCOc2ccc([C@H]3c4[nH]c5ccc(Cl)cc5c4CCN3C(=O)OCC3CC3)cc2)O1] | 1314 |
| 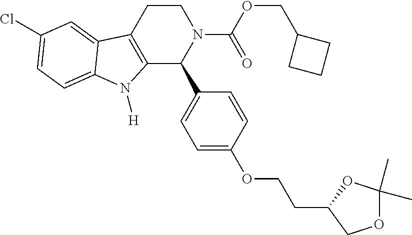 [SMILES: CC1(C)OC[C@H](CCOc2ccc([C@H]3c4[nH]c5ccc(Cl)cc5c4CCN3C(=O)OCC3CCC3)cc2)O1] | 1315 |
| 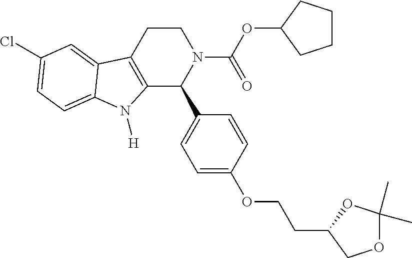 [SMILES: CC1(C)OC[C@H](CCOc2ccc([C@H]3c4[nH]c5ccc(Cl)cc5c4CCN3C(=O)OC3CCCC3)cc2)O1] | 1316 |
| 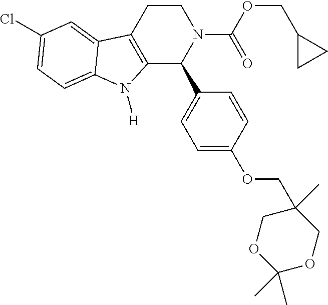 [SMILES: CC1(COc2ccc([C@H]3c4[nH]c5ccc(Cl)cc5c4CCN3C(=O)OCC3CC3)cc2)COC(C)(C)OC1] | 1317 |
| 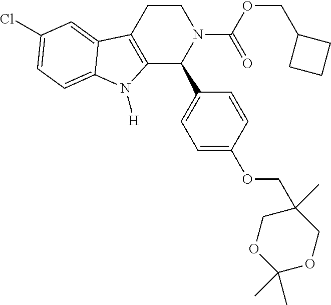 [SMILES: CC1(COc2ccc([C@H]3c4[nH]c5ccc(Cl)cc5c4CCN3C(=O)OCC3CCC3)cc2)COC(C)(C)OC1] | 1318 |
| 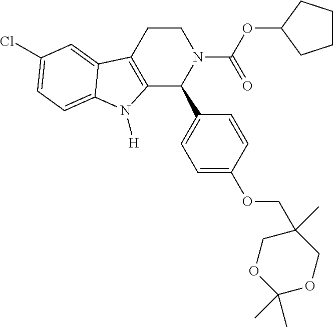 [SMILES: CC1(COc2ccc([C@H]3c4[nH]c5ccc(Cl)cc5c4CCN3C(=O)OC3CCCC3)cc2)COC(C)(C)OC1] | 1319 |
| 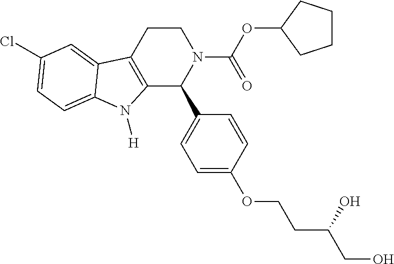 [SMILES: O=C(OC1CCCC1)N1CCc2c([nH]c3ccc(Cl)cc23)[C@@H]1c1ccc(OCC[C@H](O)CO)cc1] | 1320 |
| 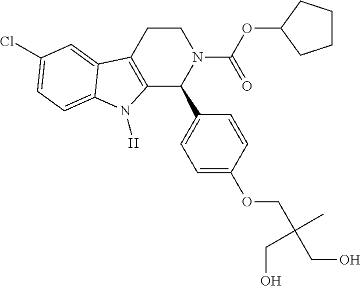 [SMILES: CC(CO)(CO)COc1ccc([C@H]2c3[nH]c4ccc(Cl)cc4c3CCN2C(=O)OC2CCCC2)cc1] | 1321 |
| 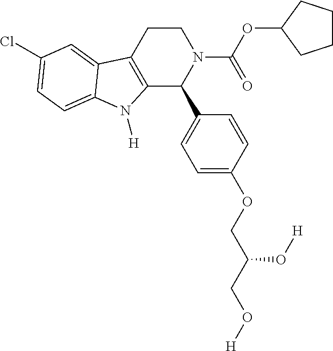 [SMILES: O=C(OC1CCCC1)N1CCc2c([nH]c3ccc(Cl)cc23)[C@@H]1c1ccc(OC[C@H](O)CO)cc1] | 1322 |
| 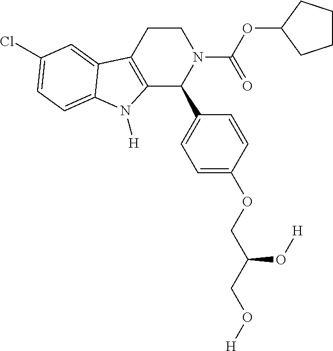 [SMILES: O=C(OC1CCCC1)N1CCc2c([nH]c3ccc(Cl)cc23)[C@@H]1c1ccc(OC[C@@H](O)CO)cc1] | 1323 |
| 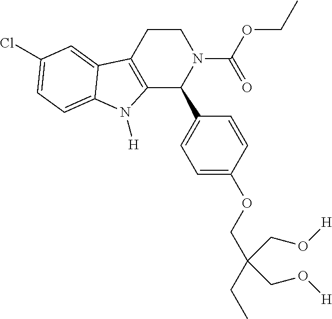 [SMILES: CCOC(=O)N1CCc2c([nH]c3ccc(Cl)cc23)[C@@H]1c1ccc(OCC(CC)(CO)CO)cc1] | 1324 |
| 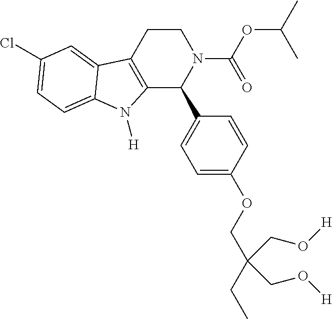 [SMILES: CCC(CO)(CO)COc1ccc([C@H]2c3[nH]c4ccc(Cl)cc4c3CCN2C(=O)OC(C)C)cc1] | 1325 |
| 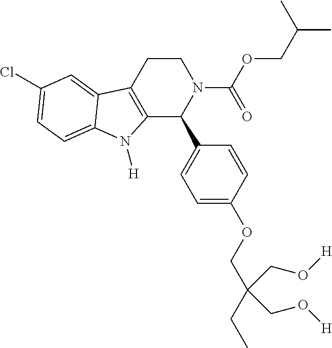 [SMILES: CCC(CO)(CO)COc1ccc([C@H]2c3[nH]c4ccc(Cl)cc4c3CCN2C(=O)OCC(C)C)cc1] | 1326 |
| 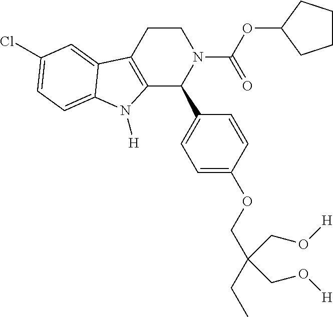 [SMILES: CCC(CO)(CO)COc1ccc([C@H]2c3[nH]c4ccc(Cl)cc4c3CCN2C(=O)OC2CCCC2)cc1] | 1327 |
| 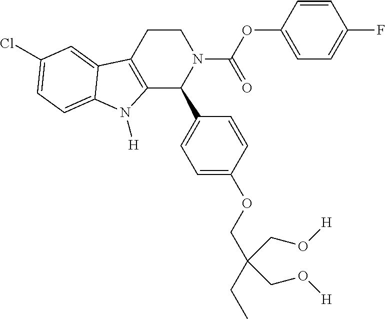 [SMILES: CCC(CO)(CO)COc1ccc([C@H]2c3[nH]c4ccc(Cl)cc4c3CCN2C(=O)Oc2ccc(F)cc2)cc1] | 1328 |
| 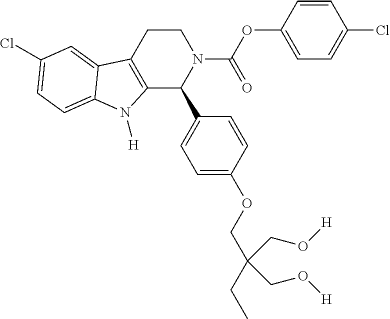 [SMILES: CCC(CO)(CO)COc1ccc([C@H]2c3[nH]c4ccc(Cl)cc4c3CCN2C(=O)Oc2ccc(Cl)cc2)cc1] | 1329 |
| 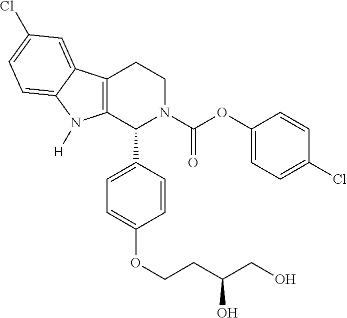 [SMILES: O=C(Oc1ccc(Cl)cc1)N1CCc2c([nH]c3ccc(Cl)cc23)[C@H]1c1ccc(OCC[C@H](O)CO)cc1] | 1330 |
| 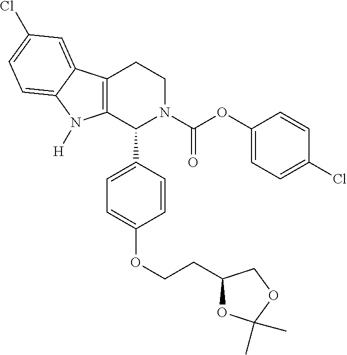 [SMILES: CC1(C)OC[C@H](CCOc2ccc([C@@H]3c4[nH]c5ccc(Cl)cc5c4CCN3C(=O)Oc3ccc(Cl)cc3)cc2)O1] | 1331 |
| 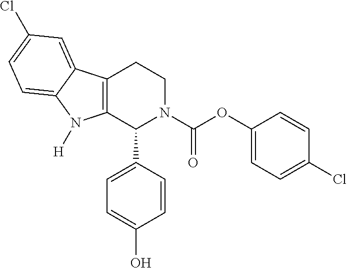 [SMILES: O=C(Oc1ccc(Cl)cc1)N1CCc2c([nH]c3ccc(Cl)cc23)[C@H]1c1ccc(O)cc1] | 1332 |
| 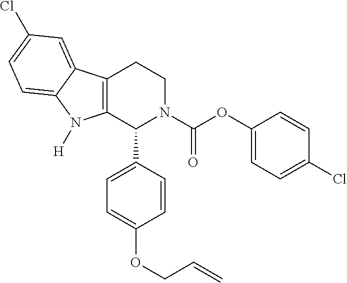 [SMILES: C=CCOc1ccc([C@@H]2c3[nH]c4ccc(Cl)cc4c3CCN2C(=O)Oc2ccc(Cl)cc2)cc1] | 1333 |
| 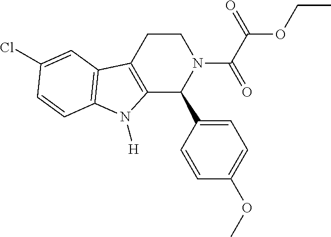 [SMILES: CCOC(=O)C(=O)N1CCc2c([nH]c3ccc(Cl)cc23)[C@@H]1c1ccc(OC)cc1] | 1334 |
| 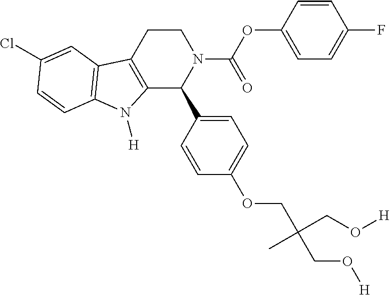 [SMILES: CC(CO)(CO)COc1ccc([C@H]2c3[nH]c4ccc(Cl)cc4c3CCN2C(=O)Oc2ccc(F)cc2)cc1] | 1335 |
| 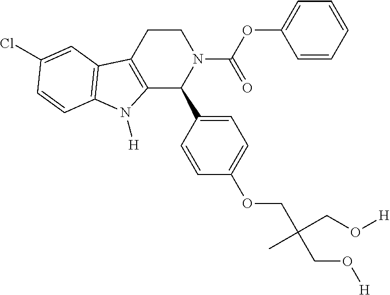 [SMILES: CC(CO)(CO)COc1ccc([C@H]2c3[nH]c4ccc(Cl)cc4c3CCN2C(=O)Oc2ccccc2)cc1] | 1336 |
| 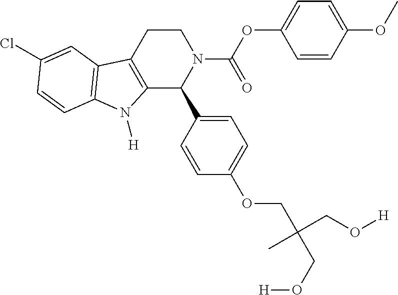 [SMILES: COc1ccc(OC(=O)N2CCc3c([nH]c4ccc(Cl)cc34)[C@@H]2c2ccc(OCC(C)(CO)CO)cc2)cc1] | 1337 |
| 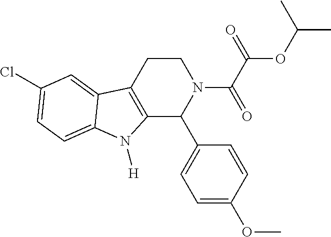 [SMILES: COc1ccc(C2c3[nH]c4ccc(Cl)cc4c3CCN2C(=O)C(=O)OC(C)C)cc1] | 1338 |
| 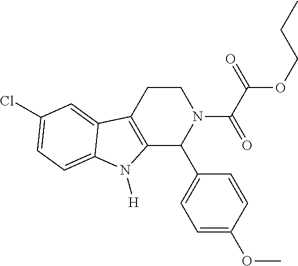 [SMILES: CCCOC(=O)C(=O)N1CCc2c([nH]c3ccc(Cl)cc23)C1c1ccc(OC)cc1] | 1339 |
| 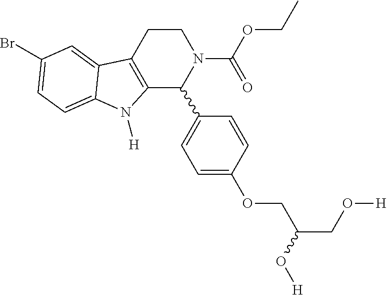 [SMILES: CCOC(=O)N1CCc2c([nH]c3ccc(Br)cc23)C1c1ccc(OCC(O)CO)cc1] | 1340 |
| 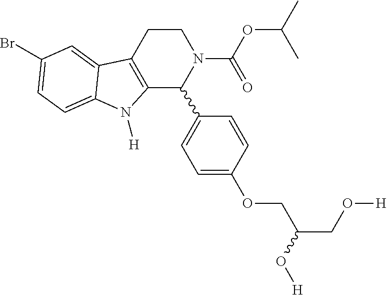 [SMILES: CC(C)OC(=O)N1CCc2c([nH]c3ccc(Br)cc23)C1c1ccc(OCC(O)CO)cc1] | 1341 |
| 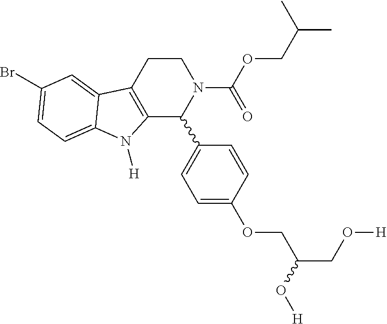 [SMILES: CC(C)COC(=O)N1CCc2c([nH]c3ccc(Br)cc23)C1c1ccc(OCC(O)CO)cc1] | 1342 |
| 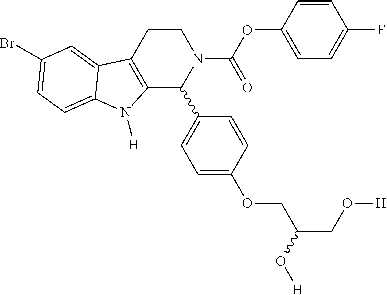 [SMILES: O=C(Oc1ccc(F)cc1)N1CCc2c([nH]c3ccc(Br)cc23)C1c1ccc(OCC(O)CO)cc1] | 1343 |
| 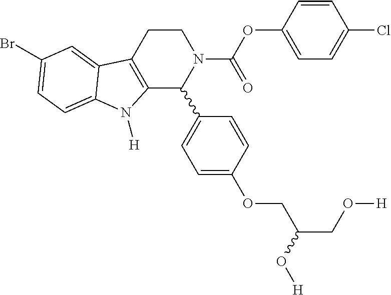 [SMILES: O=C(Oc1ccc(Cl)cc1)N1CCc2c([nH]c3ccc(Br)cc23)C1c1ccc(OCC(O)CO)cc1] | 1344 |
| 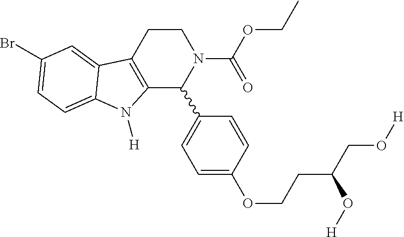 [SMILES: CCOC(=O)N1CCc2c([nH]c3ccc(Br)cc23)C1c1ccc(OCC[C@H](O)CO)cc1] | 1345 |
| 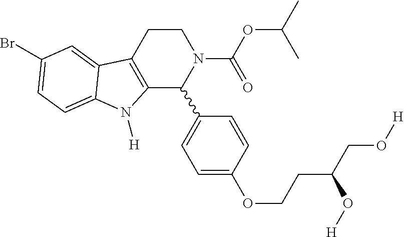 [SMILES: CC(C)OC(=O)N1CCc2c([nH]c3ccc(Br)cc23)C1c1ccc(OCC[C@H](O)CO)cc1] | 1346 |
| 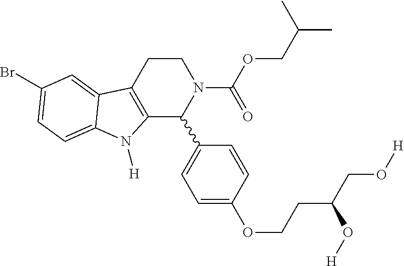 [SMILES: CC(C)COC(=O)N1CCc2c([nH]c3ccc(Br)cc23)C1c1ccc(OCC[C@H](O)CO)cc1] | 1347 |
| 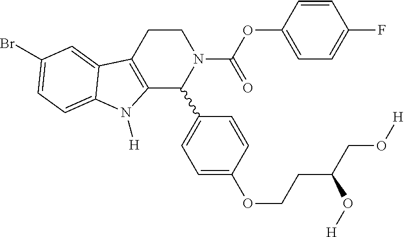 [SMILES: O=C(Oc1ccc(F)cc1)N1CCc2c([nH]c3ccc(Br)cc23)C1c1ccc(OCC[C@H](O)CO)cc1] | 1348 |
| 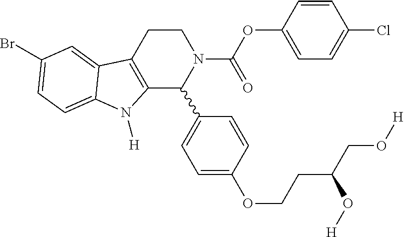 [SMILES: O=C(Oc1ccc(Cl)cc1)N1CCc2c([nH]c3ccc(Br)cc23)C1c1ccc(OCC[C@H](O)CO)cc1] | 1349 |
| 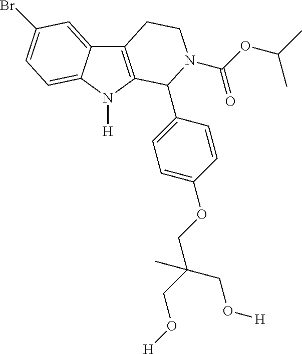 [SMILES: CC(C)OC(=O)N1CCc2c([nH]c3ccc(Br)cc23)C1c1ccc(OCC(C)(CO)CO)cc1] | 1350 |
| 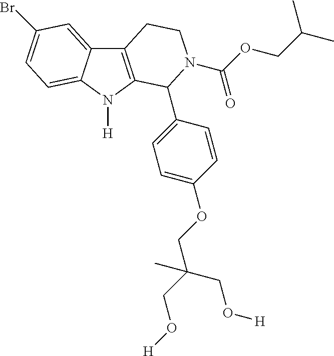 [SMILES: CC(C)COC(=O)N1CCc2c([nH]c3ccc(Br)cc23)C1c1ccc(OCC(C)(CO)CO)cc1] | 1351 |
| 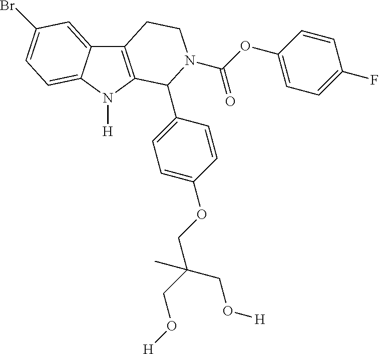 [SMILES: CC(CO)(CO)COc1ccc(C2c3[nH]c4ccc(Br)cc4c3CCN2C(=O)Oc2ccc(F)cc2)cc1] | 1352 |
| 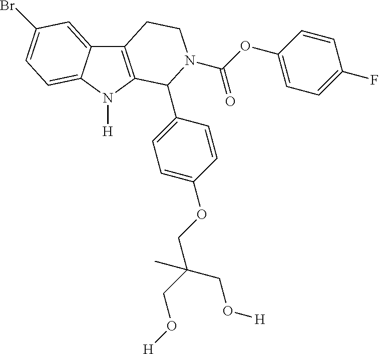 [SMILES: CC(CO)(CO)COc1ccc(C2c3[nH]c4ccc(Br)cc4c3CCN2C(=O)Oc2ccc(F)cc2)cc1] | 1353 |
| 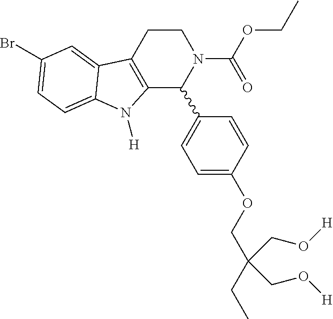 [SMILES: CCOC(=O)N1CCc2c([nH]c3ccc(Br)cc23)C1c1ccc(OCC(CC)(CO)CO)cc1] | 1354 |
| 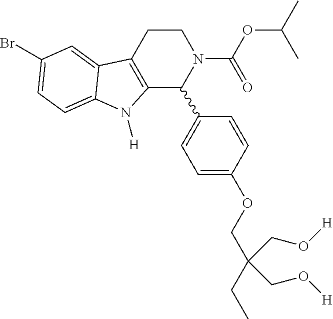 [SMILES: CCC(CO)(CO)COc1ccc(C2c3[nH]c4ccc(Br)cc4c3CCN2C(=O)OC(C)C)cc1] | 1355 |
| 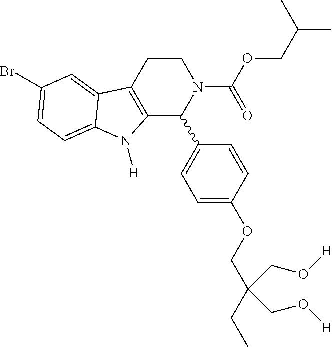 [SMILES: CCC(CO)(CO)COc1ccc(C2c3[nH]c4ccc(Br)cc4c3CCN2C(=O)OCC(C)C)cc1] | 1356 |
| 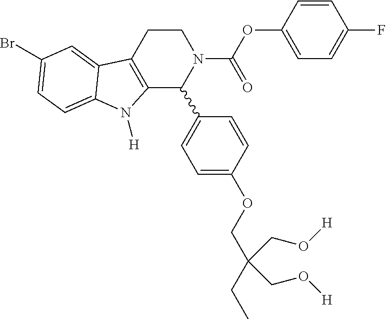 [SMILES: CCC(CO)(CO)COc1ccc(C2c3[nH]c4ccc(Br)cc4c3CCN2C(=O)Oc2ccc(F)cc2)cc1] | 1357 |
| 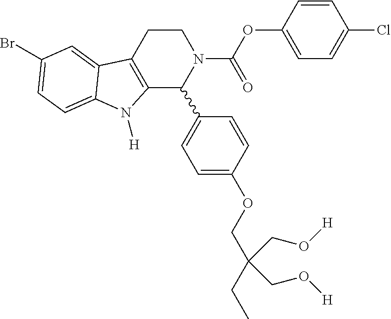 [SMILES: CCC(CO)(CO)COc1ccc(C2c3[nH]c4ccc(Br)cc4c3CCN2C(=O)Oc2ccc(Cl)cc2)cc1] | 1358 |
| 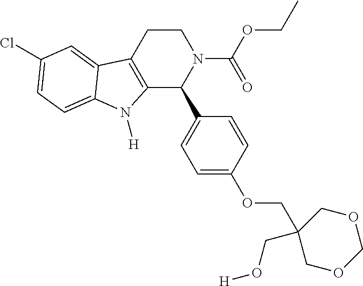 [SMILES: CCOC(=O)N1CCc2c([nH]c3ccc(Cl)cc23)[C@@H]1c1ccc(OCC2(CO)COCOC2)cc1] | 1359 |
| 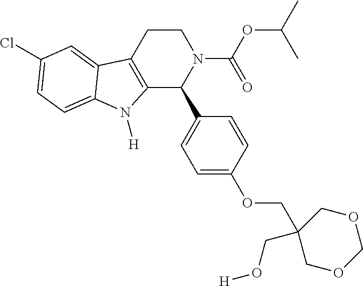 [SMILES: CC(C)OC(=O)N1CCc2c([nH]c3ccc(Cl)cc23)[C@@H]1c1ccc(OCC2(CO)COCOC2)cc1] | 1360 |
| 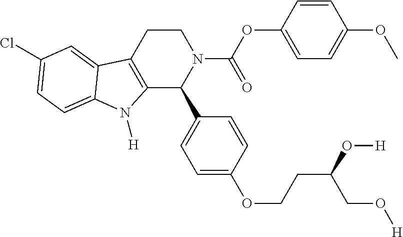 [SMILES: COc1ccc(OC(=O)N2CCc3c([nH]c4ccc(Cl)cc34)[C@@H]2c2ccc(OCC[C@@H](O)CO)cc2)cc1] | 1361 |
| 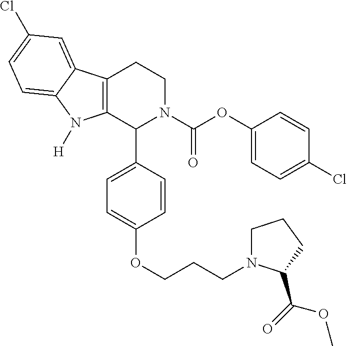 | 1362 |
| 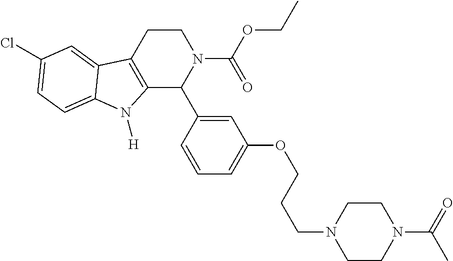 [SMILES: CCOC(=O)N1CCc2c([nH]c3ccc(Cl)cc23)C1c1cccc(OCCCN2CCN(C(C)=O)CC2)c1] | 1363 |
| 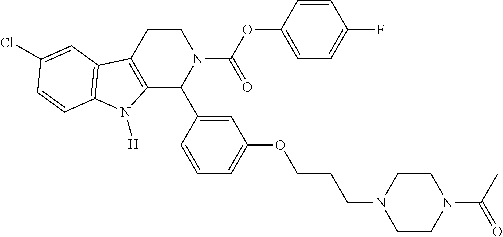 [SMILES: CC(=O)N1CCN(CCCOc2cccc(C3c4[nH]c5ccc(Cl)cc5c4CCN3C(=O)Oc3ccc(F)cc3)c2)CC1] | 1364 |
| 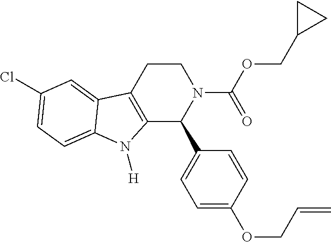 [SMILES: C=CCOc1ccc([C@H]2c3[nH]c4ccc(Cl)cc4c3CCN2C(=O)OCC2CC2)cc1] | 1365 |
| 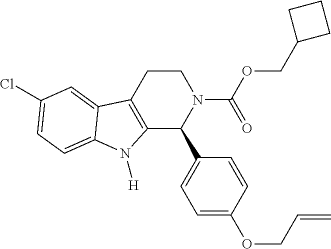 [SMILES: C=CCOc1ccc([C@H]2c3[nH]c4ccc(Cl)cc4c3CCN2C(=O)OCC2CCC2)cc1] | 1366 |
| 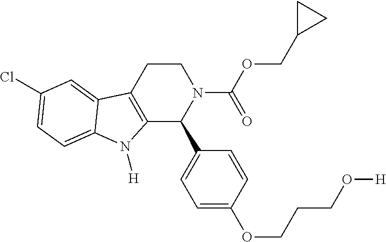 [SMILES: O=C(OCC1CC1)N1CCc2c([nH]c3ccc(Cl)cc23)[C@@H]1c1ccc(OCCCO)cc1] | 1367 |
| 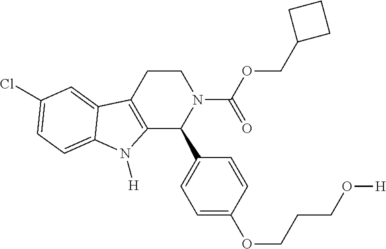 [SMILES: O=C(OCC1CCC1)N1CCc2c([nH]c3ccc(Cl)cc23)[C@@H]1c1ccc(OCCCO)cc1] | 1368 |
| 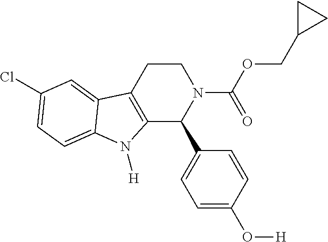 [SMILES: O=C(OCC1CC1)N1CCc2c([nH]c3ccc(Cl)cc23)[C@@H]1c1ccc(O)cc1] | 1369 |
| 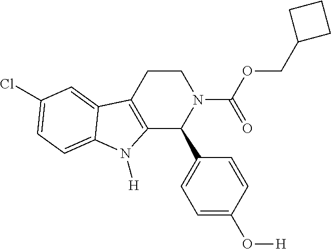 [SMILES: O=C(OCC1CCC1)N1CCc2c([nH]c3ccc(Cl)cc23)[C@@H]1c1ccc(O)cc1] | 1370 |
| 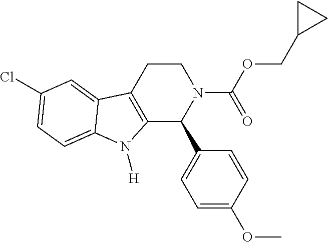 [SMILES: COc1ccc([C@H]2c3[nH]c4ccc(Cl)cc4c3CCN2C(=O)OCC2CC2)cc1] | 1371 |
| 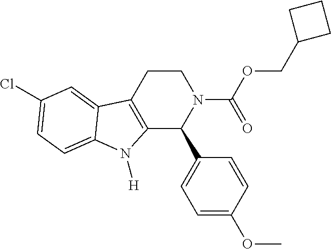 [SMILES: COc1ccc([C@H]2c3[nH]c4ccc(Cl)cc4c3CCN2C(=O)OCC2CCC2)cc1] | 1372 |
| 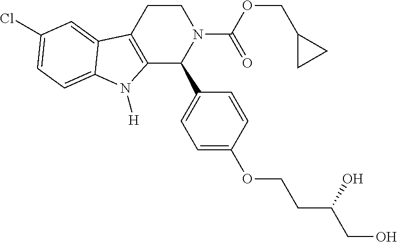 [SMILES: O=C(OCC1CC1)N1CCc2c([nH]c3ccc(Cl)cc23)[C@@H]1c1ccc(OCC[C@H](O)CO)cc1] | 1373 |
| 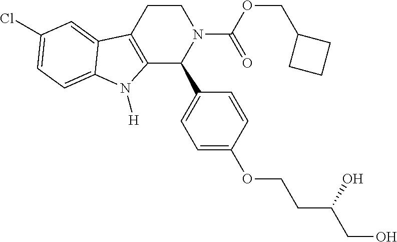 [SMILES: O=C(OCC1CCC1)N1CCc2c([nH]c3ccc(Cl)cc23)[C@@H]1c1ccc(OCC[C@H](O)CO)cc1] | 1374 |
| 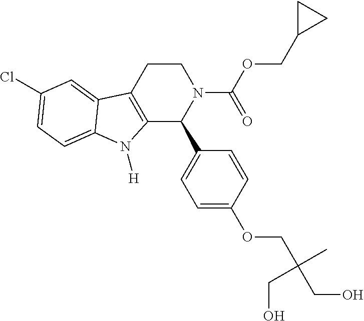 [SMILES: CC(CO)(CO)COc1ccc([C@H]2c3[nH]c4ccc(Cl)cc4c3CCN2C(=O)OCC2CC2)cc1] | 1375 |
| 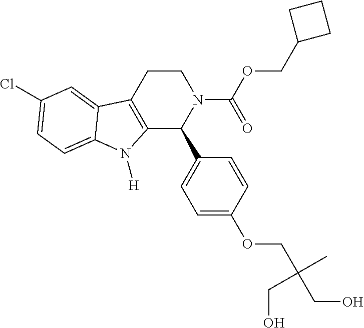 [SMILES: CC(CO)(CO)COc1ccc([C@H]2c3[nH]c4ccc(Cl)cc4c3CCN2C(=O)OCC2CCC2)cc1] | 1376 |
| 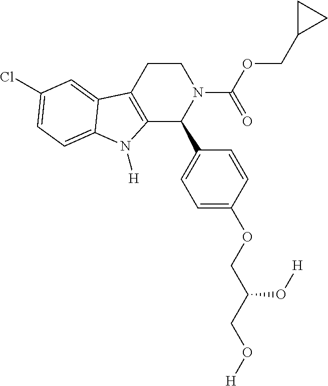 [SMILES: O=C(OCC1CC1)N1CCc2c([nH]c3ccc(Cl)cc23)[C@@H]1c1ccc(OC[C@H](O)CO)cc1] | 1377 |
| 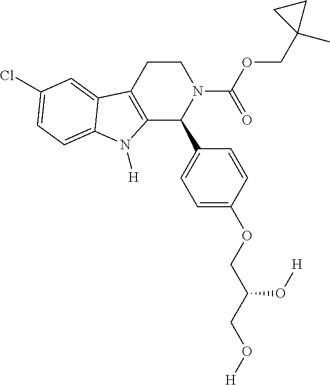 [SMILES: CC1(COC(=O)N2CCc3c([nH]c4ccc(Cl)cc34)[C@@H]2c2ccc(OC[C@H](O)CO)cc2)CC1] | 1378 |
| 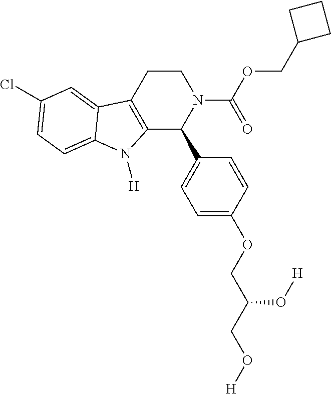 [SMILES: O=C(OCC1CCC1)N1CCc2c([nH]c3ccc(Cl)cc23)[C@@H]1c1ccc(OC[C@H](O)CO)cc1] | 1379 |
| 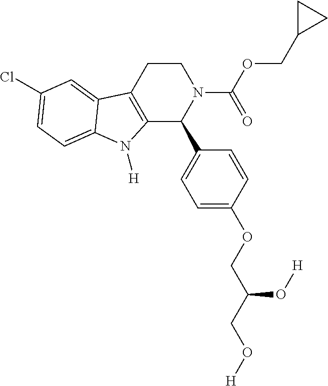 [SMILES: O=C(OCC1CC1)N1CCc2c([nH]c3ccc(Cl)cc23)[C@@H]1c1ccc(OC[C@@H](O)CO)cc1] | 1380 |
| 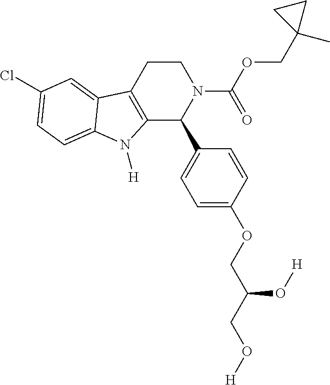 [SMILES: CC1(COC(=O)N2CCc3c([nH]c4ccc(Cl)cc34)[C@@H]2c2ccc(OC[C@@H](O)CO)cc2)CC1] | 1381 |
| 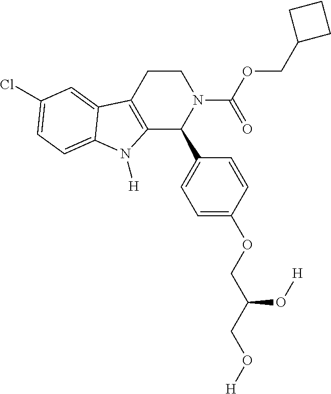 [SMILES: O=C(OCC1CCC1)N1CCc2c([nH]c3ccc(Cl)cc23)[C@@H]1c1ccc(OC[C@@H](O)CO)cc1] | 1382 |
| 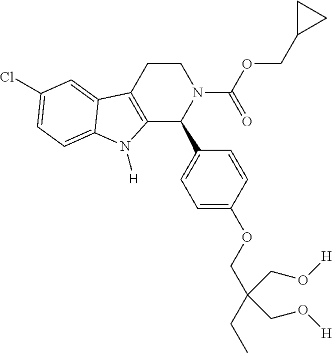 [SMILES: CCC(CO)(CO)COc1ccc([C@H]2c3[nH]c4ccc(Cl)cc4c3CCN2C(=O)OCC2CC2)cc1] | 1383 |
| 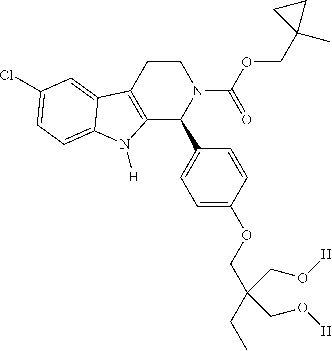 [SMILES: CCC(CO)(CO)COc1ccc([C@H]2c3[nH]c4ccc(Cl)cc4c3CCN2C(=O)OCC2(C)CC2)cc1] | 1384 |
| 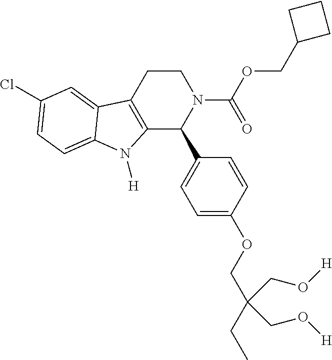 [SMILES: CCC(CO)(CO)COc1ccc([C@H]2c3[nH]c4ccc(Cl)cc4c3CCN2C(=O)OCC2CCC2)cc1] | 1385 |
| 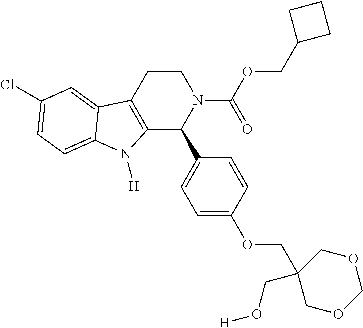 [SMILES: O=C(OCC1CCC1)N1CCc2c([nH]c3ccc(Cl)cc23)[C@@H]1c1ccc(OCC2(CO)COCOC2)cc1] | 1386 |
| 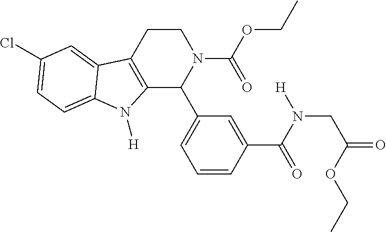 [SMILES: CCOC(=O)CNC(=O)c1cccc(C2c3[nH]c4ccc(Cl)cc4c3CCN2C(=O)OCC)c1] | 1387 |
| 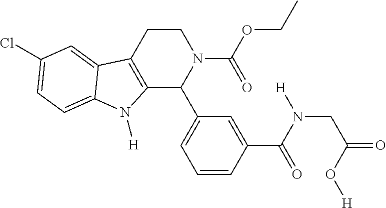 [SMILES: CCOC(=O)N1CCc2c([nH]c3ccc(Cl)cc23)C1c1cccc(C(=O)NCC(=O)O)c1] | 1388 |
| 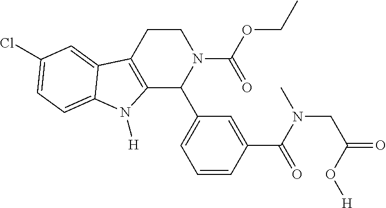 [SMILES: CCOC(=O)N1CCc2c([nH]c3ccc(Cl)cc23)C1c1cccc(C(=O)N(C)CC(=O)O)c1] | 1389 |
| 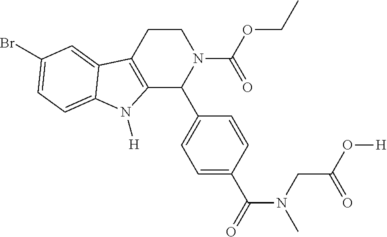 [SMILES: CCOC(=O)N1CCc2c([nH]c3ccc(Br)cc23)C1c1ccc(C(=O)N(C)CC(=O)O)cc1] | 1390 |
| 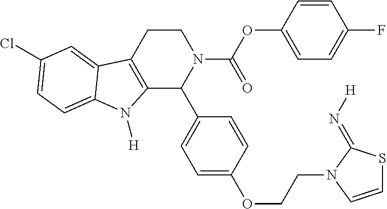 [SMILES: [H]/N=c1\sccn1CCOc1ccc(C2c3[nH]c4ccc(Cl)cc4c3CCN2C(=O)Oc2ccc(F)cc2)cc1] | 1391 |
| 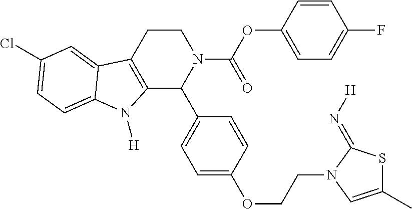 [SMILES: [H]/N=c1\sc(C)cn1CCOc1ccc(C2c3[nH]c4ccc(Cl)cc4c3CCN2C(=O)Oc2ccc(F)cc2)cc1] | 1392 |
| 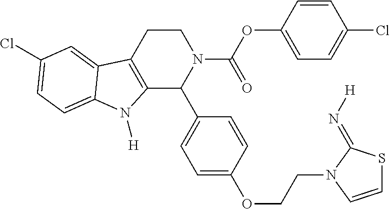 [SMILES: [H]/N=c1\sccn1CCOc1ccc(C2c3[nH]c4ccc(Cl)cc4c3CCN2C(=O)Oc2ccc(Cl)cc2)cc1] | 1393 |
| 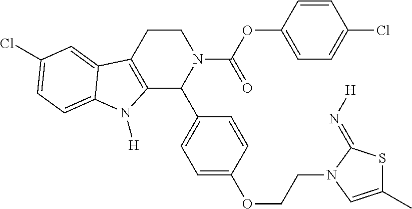 [SMILES: [H]/N=c1\sc(C)cn1CCOc1ccc(C2c3[nH]c4ccc(Cl)cc4c3CCN2C(=O)Oc2ccc(Cl)cc2)cc1] | 1394 |
| 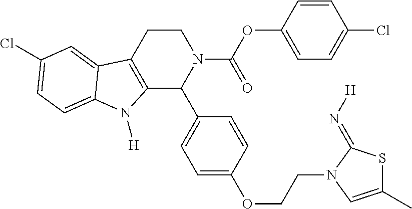 [SMILES: [H]/N=c1\sc(C)cn1CCOc1ccc(C2c3[nH]c4ccc(Cl)cc4c3CCN2C(=O)Oc2ccc(Cl)cc2)cc1] | 1395 |
| 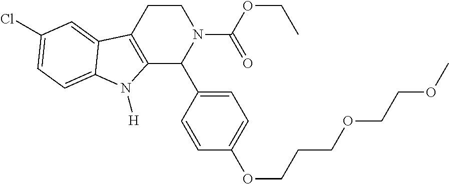 [SMILES: CCOC(=O)N1CCc2c([nH]c3ccc(Cl)cc23)C1c1ccc(OCCCOCCOC)cc1] | 1396 |
| 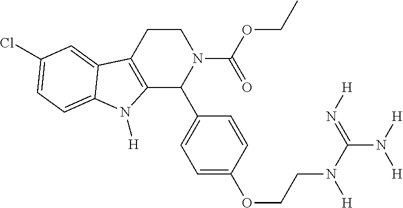 [SMILES: [H]/N=C(\N)NCCOc1ccc(C2c3[nH]c4ccc(Cl)cc4c3CCN2C(=O)OCC)cc1] | 1397 |
| 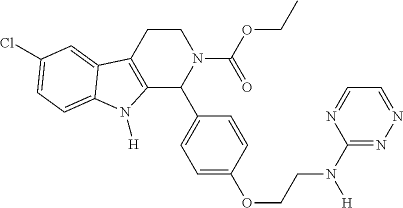 [SMILES: CCOC(=O)N1CCc2c([nH]c3ccc(Cl)cc23)C1c1ccc(OCCNc2nccnn2)cc1] | 1398 |
| 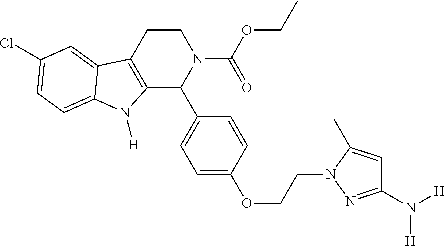 [SMILES: CCOC(=O)N1CCc2c([nH]c3ccc(Cl)cc23)C1c1ccc(OCCn2nc(N)cc2C)cc1] | 1399 |
| 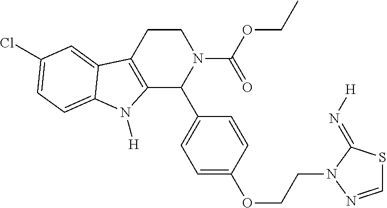 [SMILES: [H]/N=c1\scnn1CCOc1ccc(C2c3[nH]c4ccc(Cl)cc4c3CCN2C(=O)OCC)cc1] | 1400 |
| 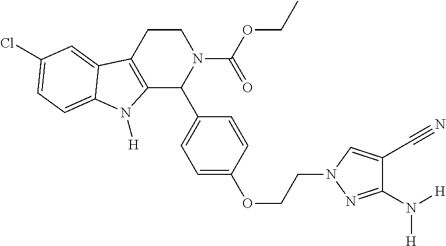 [SMILES: CCOC(=O)N1CCc2c([nH]c3ccc(Cl)cc23)C1c1ccc(OCCn2cc(C#N)c(N)n2)cc1] | 1401 |
| 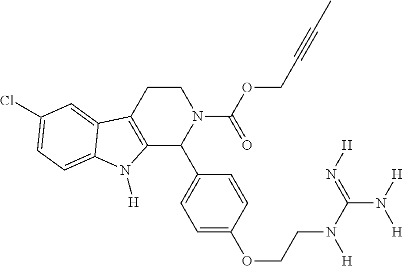 [SMILES: [H]/N=C(\N)NCCOc1ccc(C2c3[nH]c4ccc(Cl)cc4c3CCN2C(=O)OCC#CC)cc1] | 1402 |
| 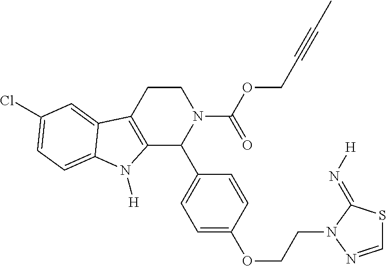 [SMILES: [H]/N=c1\scnn1CCOc1ccc(C2c3[nH]c4ccc(Cl)cc4c3CCN2C(=O)OCC#CC)cc1] | 1403 |
| 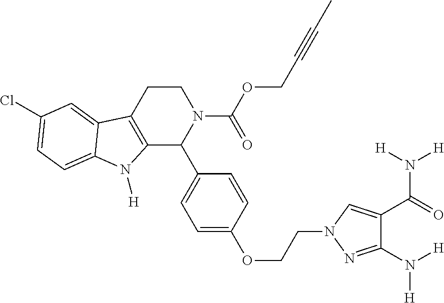 [SMILES: CC#CCOC(=O)N1CCc2c([nH]c3ccc(Cl)cc23)C1c1ccc(OCCn2cc(C(N)=O)c(N)n2)cc1] | 1404 |
| 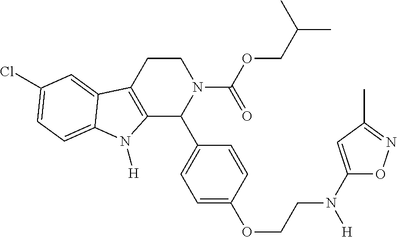 [SMILES: Cc1cc(NCCOc2ccc(C3c4[nH]c5ccc(Cl)cc5c4CCN3C(=O)OCC(C)C)cc2)on1] | 1405 |
| 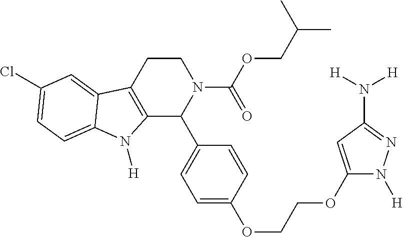 [SMILES: CC(C)COC(=O)N1CCc2c([nH]c3ccc(Cl)cc23)C1c1ccc(OCCOc2cc(N)n[nH]2)cc1] | 1406 |
| 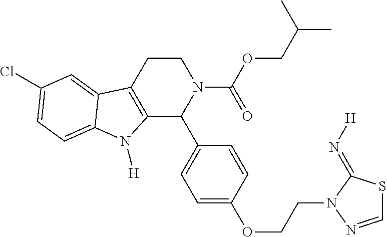 [SMILES: [H]/N=c1\scnn1CCOc1ccc(C2c3[nH]c4ccc(Cl)cc4c3CCN2C(=O)OCC(C)C)cc1] | 1407 |
| 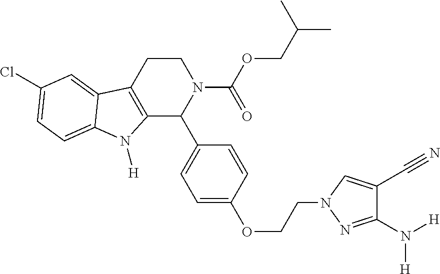 [SMILES: CC(C)COC(=O)N1CCc2c([nH]c3ccc(Cl)cc23)C1c1ccc(OCCn2cc(C#N)c(N)n2)cc1] | 1408 |
| 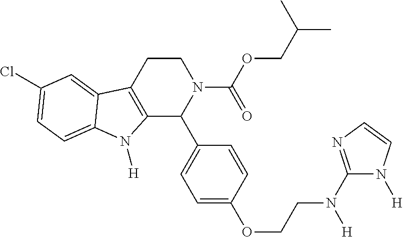 [SMILES: CC(C)COC(=O)N1CCc2c([nH]c3ccc(Cl)cc23)C1c1ccc(OCCNc2ncc[nH]2)cc1] | 1409 |
| 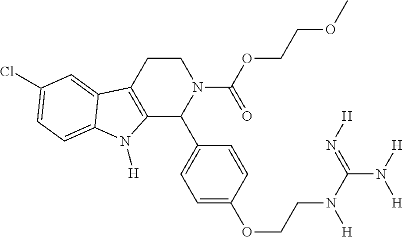 [SMILES: [H]/N=C(\N)NCCOc1ccc(C2c3[nH]c4ccc(Cl)cc4c3CCN2C(=O)OCCOC)cc1] | 1410 |
| 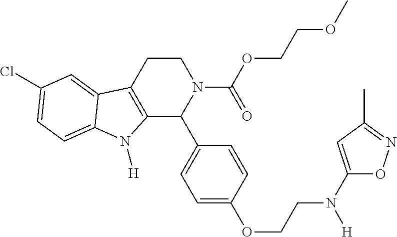 [SMILES: COCCOC(=O)N1CCc2c([nH]c3ccc(Cl)cc23)C1c1ccc(OCCNc2cc(C)no2)cc1] | 1411 |
| 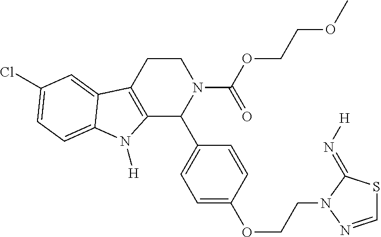 [SMILES: [H]/N=c1\scnn1CCOc1ccc(C2c3[nH]c4ccc(Cl)cc4c3CCN2C(=O)OCCOC)cc1] | 1412 |
| 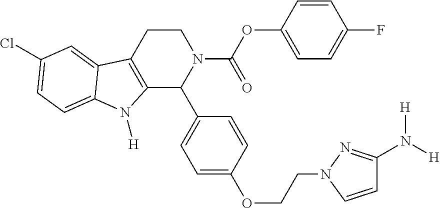 [SMILES: Nc1ccn(CCOc2ccc(C3c4[nH]c5ccc(Cl)cc5c4CCN3C(=O)Oc3ccc(F)cc3)cc2)n1] | 1413 |
| 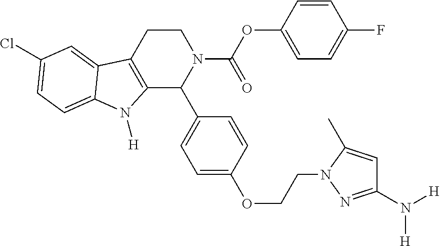 [SMILES: Cc1cc(N)nn1CCOc1ccc(C2c3[nH]c4ccc(Cl)cc4c3CCN2C(=O)Oc2ccc(F)cc2)cc1] | 1414 |
| 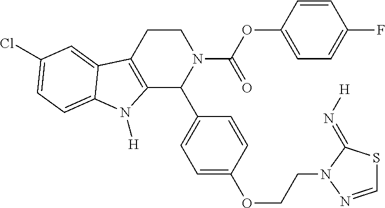 [SMILES: [H]/N=c1\scnn1CCOc1ccc(C2c3[nH]c4ccc(Cl)cc4c3CCN2C(=O)Oc2ccc(F)cc2)cc1] | 1415 |
| 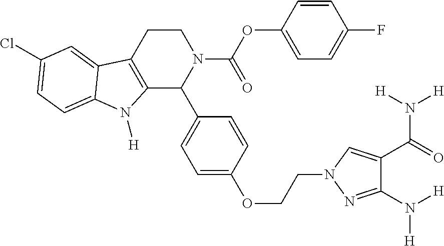 [SMILES: NC(=O)c1cn(CCOc2ccc(C3c4[nH]c5ccc(Cl)cc5c4CCN3C(=O)Oc3ccc(F)cc3)cc2)nc1N] | 1416 |
| 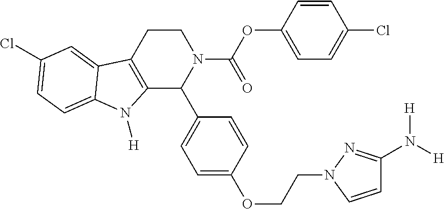 [SMILES: Nc1ccn(CCOc2ccc(C3c4[nH]c5ccc(Cl)cc5c4CCN3C(=O)Oc3ccc(Cl)cc3)cc2)n1] | 1417 |
| 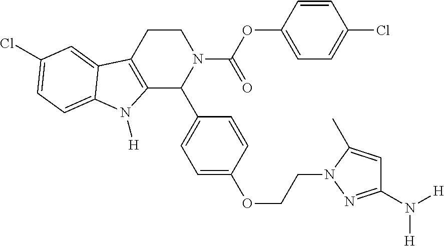 [SMILES: Cc1cc(N)nn1CCOc1ccc(C2c3[nH]c4ccc(Cl)cc4c3CCN2C(=O)Oc2ccc(Cl)cc2)cc1] | 1418 |
| 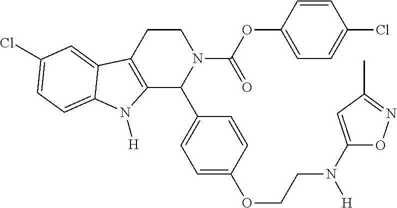 [SMILES: Cc1cc(NCCOc2ccc(C3c4[nH]c5ccc(Cl)cc5c4CCN3C(=O)Oc3ccc(Cl)cc3)cc2)on1] | 1419 |
| 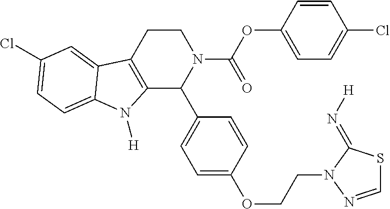 [SMILES: [H]/N=c1\scnn1CCOc1ccc(C2c3[nH]c4ccc(Cl)cc4c3CCN2C(=O)Oc2ccc(Cl)cc2)cc1] | 1420 |
| 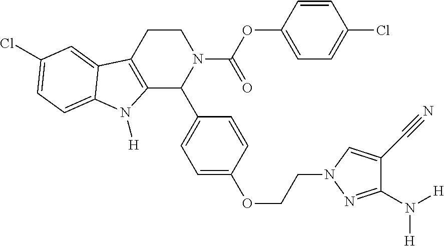 [SMILES: N#Cc1cn(CCOc2ccc(C3c4[nH]c5ccc(Cl)cc5c4CCN3C(=O)Oc3ccc(Cl)cc3)cc2)nc1N] | 1421 |
| 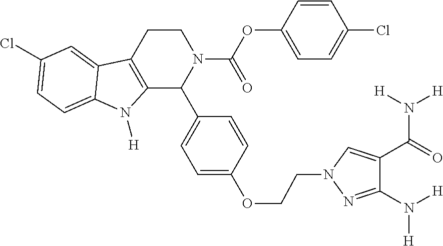 [SMILES: NC(=O)c1cn(CCOc2ccc(C3c4[nH]c5ccc(Cl)cc5c4CCN3C(=O)Oc3ccc(Cl)cc3)cc2)nc1N] | 1422 |
| 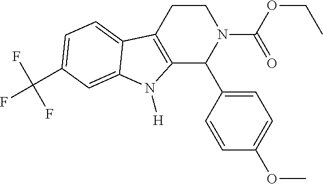 [SMILES: CCOC(=O)N1CCc2c([nH]c3cc(C(F)(F)F)ccc23)C1c1ccc(OC)cc1] | 1423 |
| 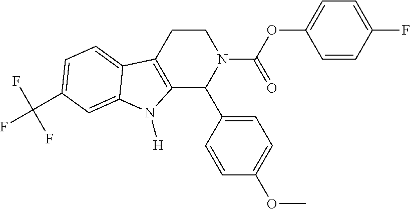 [SMILES: COc1ccc(C2c3[nH]c4cc(C(F)(F)F)ccc4c3CCN2C(=O)Oc2ccc(F)cc2)cc1] | 1424 |
| 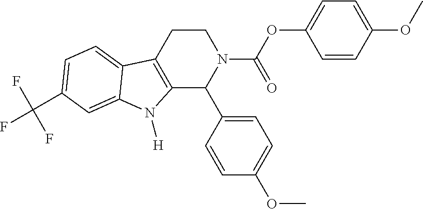 [SMILES: COc1ccc(OC(=O)N2CCc3c([nH]c4cc(C(F)(F)F)ccc34)C2c2ccc(OC)cc2)cc1] | 1425 |
| 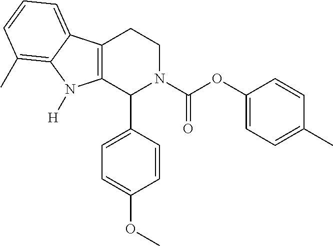 | 1426 |
| 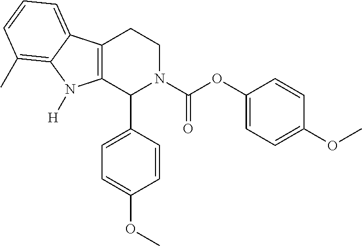 | 1427 |
| 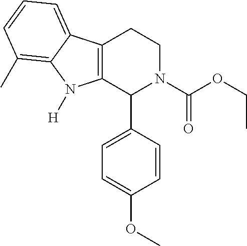 [SMILES: CCOC(=O)N1CCc2c([nH]c3c(C)cccc23)C1c1ccc(OC)cc1] | 1428 |
| 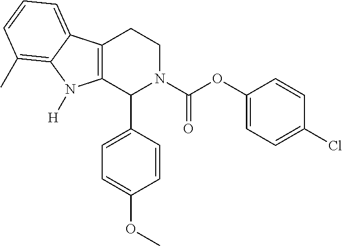 [SMILES: COc1ccc(C2c3[nH]c4c(C)cccc4c3CCN2C(=O)Oc2ccc(Cl)cc2)cc1] | 1429 |
| 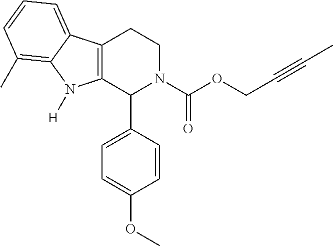 [SMILES: CC#CCOC(=O)N1CCc2c([nH]c3c(C)cccc23)C1c1ccc(OC)cc1] | 1430 |
| 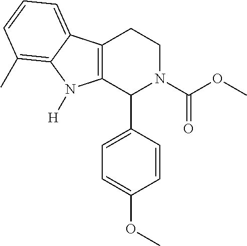 | 1431 |
| 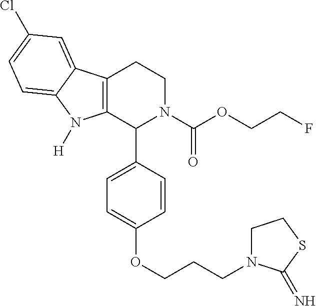 [SMILES: N=C1SCCN1CCCOc1ccc(C2c3[nH]c4ccc(Cl)cc4c3CCN2C(=O)OCCF)cc1] | 1432 |
| 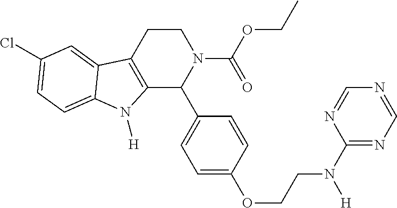 [SMILES: CCOC(=O)N1CCc2c([nH]c3ccc(Cl)cc23)C1c1ccc(OCCNc2ncncn2)cc1] | 1433 |
| 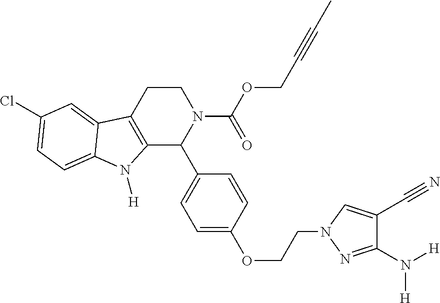 [SMILES: CC#CCOC(=O)N1CCc2c([nH]c3ccc(Cl)cc23)C1c1ccc(OCCn2cc(C#N)c(N)n2)cc1] | 1434 |
| 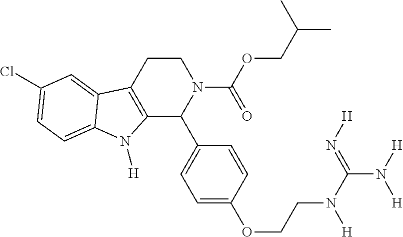 [SMILES: [H]/N=C(\N)NCCOc1ccc(C2c3[nH]c4ccc(Cl)cc4c3CCN2C(=O)OCC(C)C)cc1] | 1435 |
| 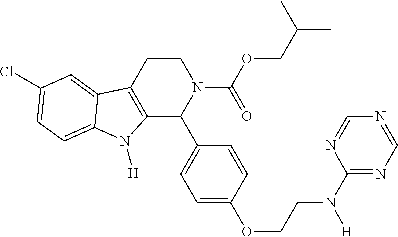 [SMILES: CC(C)COC(=O)N1CCc2c([nH]c3ccc(Cl)cc23)C1c1ccc(OCCNc2ncncn2)cc1] | 1436 |
| 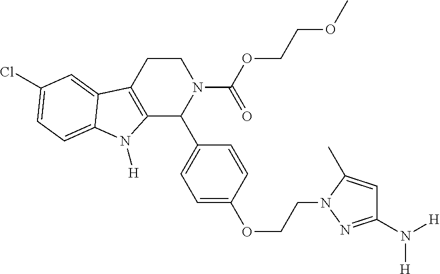 [SMILES: COCCOC(=O)N1CCc2c([nH]c3ccc(Cl)cc23)C1c1ccc(OCCn2nc(N)cc2C)cc1] | 1437 |
| 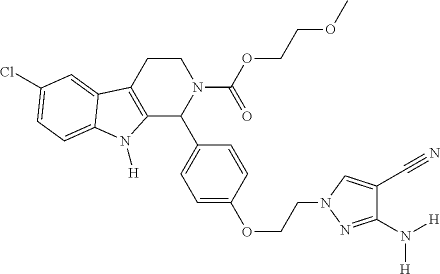 [SMILES: COCCOC(=O)N1CCc2c([nH]c3ccc(Cl)cc23)C1c1ccc(OCCn2cc(C#N)c(N)n2)cc1] | 1438 |
| 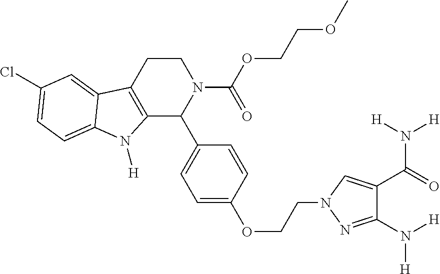 [SMILES: COCCOC(=O)N1CCc2c([nH]c3ccc(Cl)cc23)C1c1ccc(OCCn2cc(C(N)=O)c(N)n2)cc1] | 1439 |
| 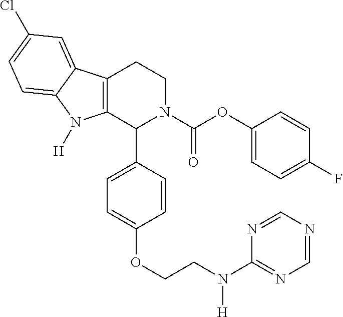 [SMILES: O=C(Oc1ccc(F)cc1)N1CCc2c([nH]c3ccc(Cl)cc23)C1c1ccc(OCCNc2ncncn2)cc1] | 1440 |
| 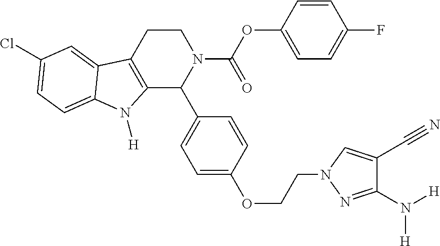 [SMILES: N#Cc1cn(CCOc2ccc(C3c4[nH]c5ccc(Cl)cc5c4CCN3C(=O)Oc3ccc(F)cc3)cc2)nc1N] | 1441 |
| 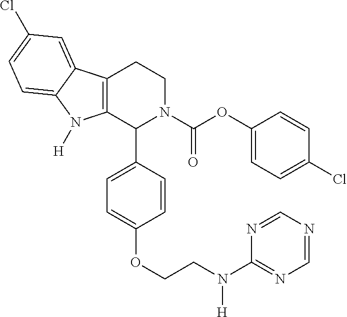 [SMILES: O=C(Oc1ccc(Cl)cc1)N1CCc2c([nH]c3ccc(Cl)cc23)C1c1ccc(OCCNc2ncncn2)cc1] | 1442 |
| 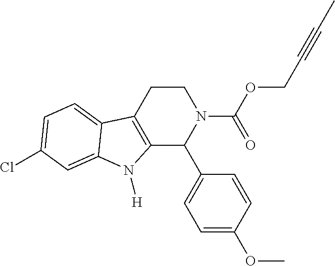 [SMILES: CC#CCOC(=O)N1CCc2c([nH]c3cc(Cl)ccc23)C1c1ccc(OC)cc1] | 1443 |
| 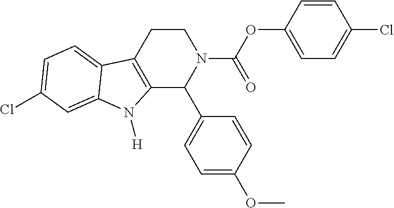 [SMILES: COc1ccc(C2c3[nH]c4cc(Cl)ccc4c3CCN2C(=O)Oc2ccc(Cl)cc2)cc1] | 1444 |
| 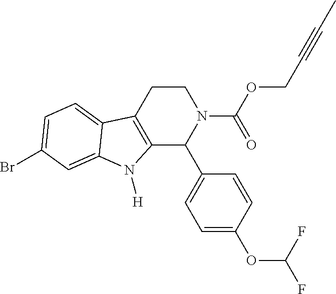 [SMILES: CC#CCOC(=O)N1CCc2c([nH]c3cc(Br)ccc23)C1c1ccc(OC(F)F)cc1] | 1445 |
| 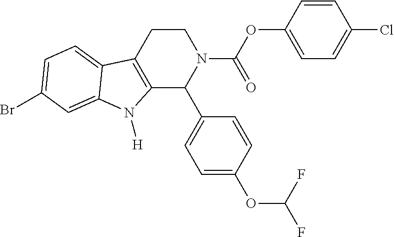 [SMILES: O=C(Oc1ccc(Cl)cc1)N1CCc2c([nH]c3cc(Br)ccc23)C1c1ccc(OC(F)F)cc1] | 1446 |
| 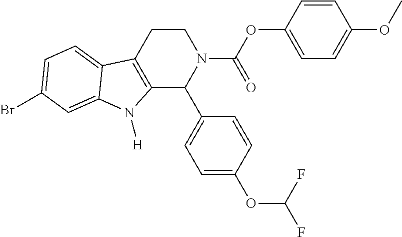 [SMILES: COc1ccc(OC(=O)N2CCc3c([nH]c4cc(Br)ccc34)C2c2ccc(OC(F)F)cc2)cc1] | 1447 |
| 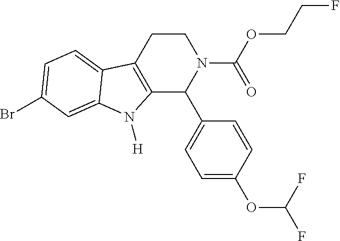 [SMILES: O=C(OCCF)N1CCc2c([nH]c3cc(Br)ccc23)C1c1ccc(OC(F)F)cc1] | 1448 |
| 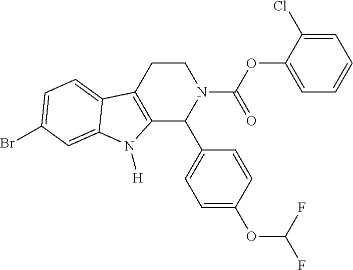 [SMILES: O=C(Oc1ccccc1Cl)N1CCc2c([nH]c3cc(Br)ccc23)C1c1ccc(OC(F)F)cc1] | 1449 |
| 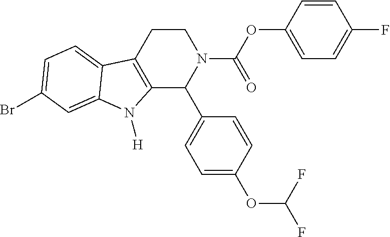 [SMILES: O=C(Oc1ccc(F)cc1)N1CCc2c([nH]c3cc(Br)ccc23)C1c1ccc(OC(F)F)cc1] | 1450 |
| 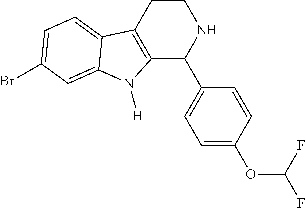 [SMILES: FC(F)Oc1ccc(C2NCCc3c2[nH]c2cc(Br)ccc32)cc1] | 1451 |
| 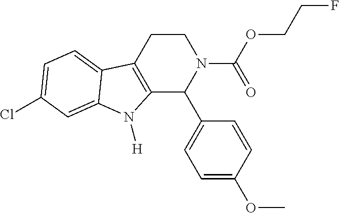 [SMILES: COc1ccc(C2c3[nH]c4cc(Cl)ccc4c3CCN2C(=O)OCCF)cc1] | 1452 |
| 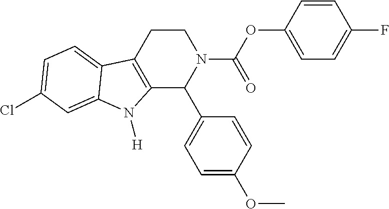 [SMILES: COc1ccc(C2c3[nH]c4cc(Cl)ccc4c3CCN2C(=O)Oc2ccc(F)cc2)cc1] | 1453 |
| 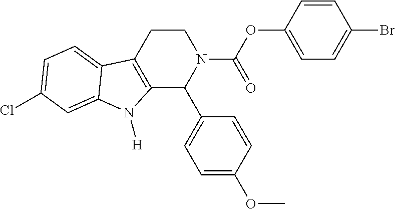 [SMILES: COc1ccc(C2c3[nH]c4cc(Cl)ccc4c3CCN2C(=O)Oc2ccc(Br)cc2)cc1] | 1454 |
| 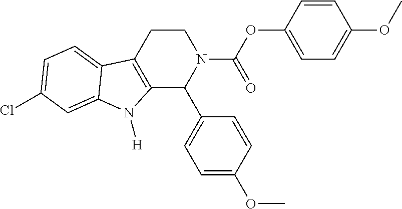 [SMILES: COc1ccc(OC(=O)N2CCc3c([nH]c4cc(Cl)ccc34)C2c2ccc(OC)cc2)cc1] | 1455 |
| 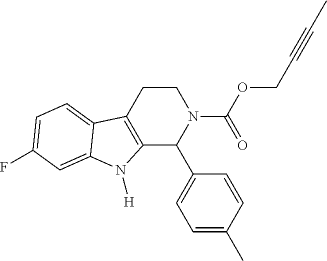 [SMILES: CC#CCOC(=O)N1CCc2c([nH]c3cc(F)ccc23)C1c1ccc(C)cc1] | 1456 |
| 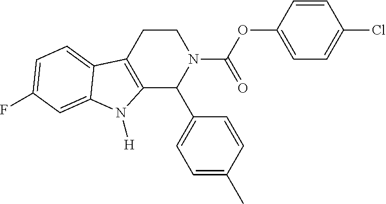 [SMILES: Cc1ccc(C2c3[nH]c4cc(F)ccc4c3CCN2C(=O)Oc2ccc(Cl)cc2)cc1] | 1457 |
| 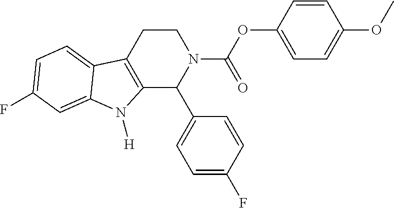 [SMILES: COc1ccc(OC(=O)N2CCc3c([nH]c4cc(F)ccc34)C2c2ccc(F)cc2)cc1] | 1458 |
| 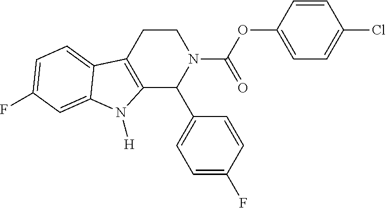 [SMILES: O=C(Oc1ccc(Cl)cc1)N1CCc2c([nH]c3cc(F)ccc23)C1c1ccc(F)cc1] | 1459 |
| 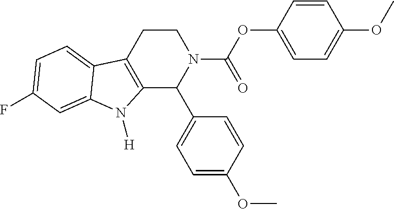 [SMILES: COc1ccc(OC(=O)N2CCc3c([nH]c4cc(F)ccc34)C2c2ccc(OC)cc2)cc1] | 1460 |
| 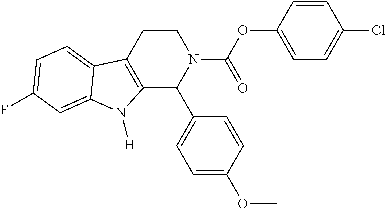 [SMILES: COc1ccc(C2c3[nH]c4cc(F)ccc4c3CCN2C(=O)Oc2ccc(Cl)cc2)cc1] | 1461 |
| 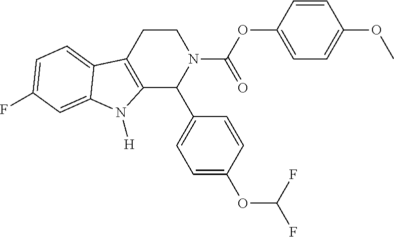 [SMILES: COc1ccc(OC(=O)N2CCc3c([nH]c4cc(F)ccc34)C2c2ccc(OC(F)F)cc2)cc1] | 1462 |
| 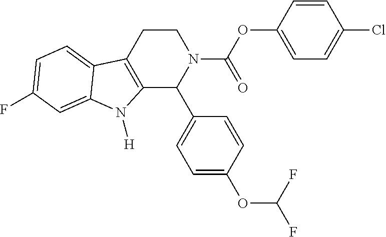 [SMILES: O=C(Oc1ccc(Cl)cc1)N1CCc2c([nH]c3cc(F)ccc23)C1c1ccc(OC(F)F)cc1] | 1463 |
| 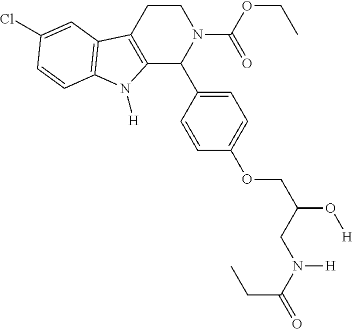 [SMILES: CCOC(=O)N1CCc2c([nH]c3ccc(Cl)cc23)C1c1ccc(OCC(O)CNC(=O)CC)cc1] | 1464 |
| 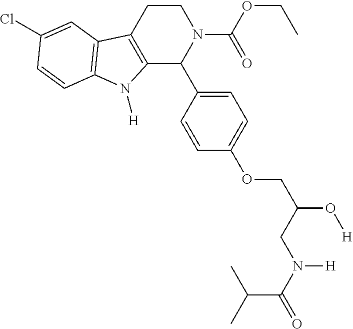 [SMILES: CCOC(=O)N1CCc2c([nH]c3ccc(Cl)cc23)C1c1ccc(OCC(O)CNC(=O)C(C)C)cc1] | 1465 |
| 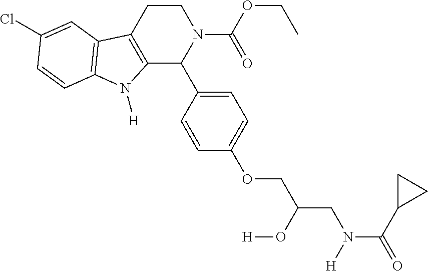 [SMILES: CCOC(=O)N1CCc2c([nH]c3ccc(Cl)cc23)C1c1ccc(OCC(O)CNC(=O)C2CC2)cc1] | 1466 |
| 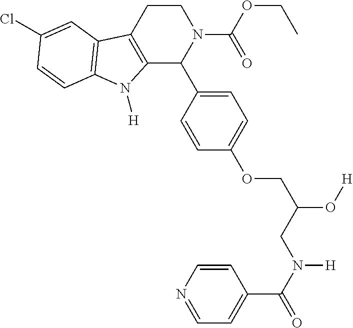 [SMILES: CCOC(=O)N1CCc2c([nH]c3ccc(Cl)cc23)C1c1ccc(OCC(O)CNC(=O)c2ccncc2)cc1] | 1467 |
| 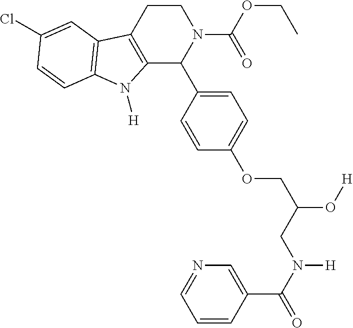 [SMILES: CCOC(=O)N1CCc2c([nH]c3ccc(Cl)cc23)C1c1ccc(OCC(O)CNC(=O)c2cccnc2)cc1] | 1468 |
| 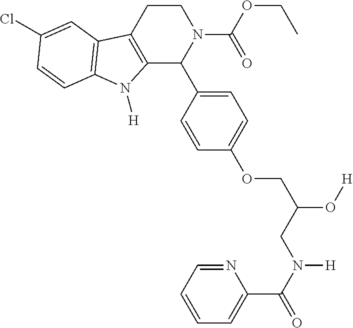 [SMILES: CCOC(=O)N1CCc2c([nH]c3ccc(Cl)cc23)C1c1ccc(OCC(O)CNC(=O)c2ccccn2)cc1] | 1469 |
| 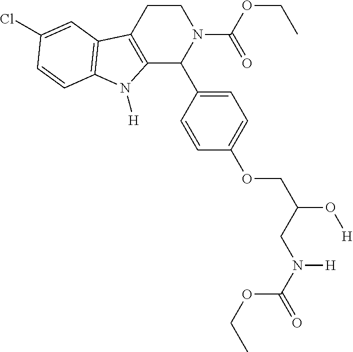 [SMILES: CCOC(=O)NCC(O)COc1ccc(C2c3[nH]c4ccc(Cl)cc4c3CCN2C(=O)OCC)cc1] | 1470 |
| 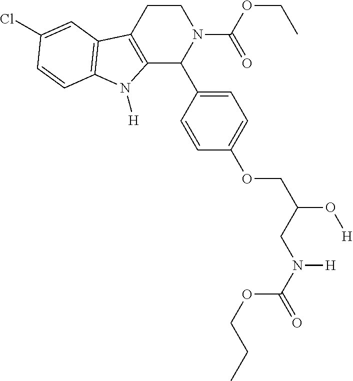 [SMILES: CCCOC(=O)NCC(O)COc1ccc(C2c3[nH]c4ccc(Cl)cc4c3CCN2C(=O)OCC)cc1] | 1471 |
| 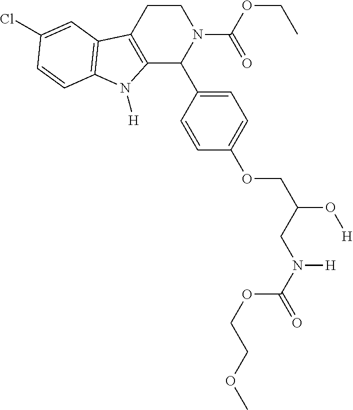 [SMILES: CCOC(=O)N1CCc2c([nH]c3ccc(Cl)cc23)C1c1ccc(OCC(O)CNC(=O)OCCOC)cc1] | 1472 |
| 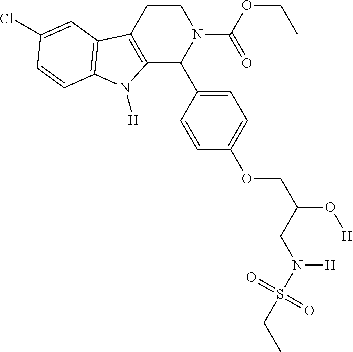 [SMILES: CCOC(=O)N1CCc2c([nH]c3ccc(Cl)cc23)C1c1ccc(OCC(O)CNS(=O)(=O)CC)cc1] | 1473 |
| 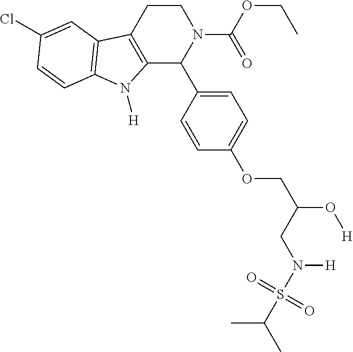 [SMILES: CCOC(=O)N1CCc2c([nH]c3ccc(Cl)cc23)C1c1ccc(OCC(O)CNS(=O)(=O)C(C)C)cc1] | 1474 |
| 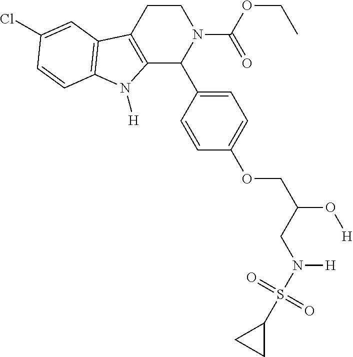 [SMILES: CCOC(=O)N1CCc2c([nH]c3ccc(Cl)cc23)C1c1ccc(OCC(O)CNS(=O)(=O)C2CC2)cc1] | 1475 |
| 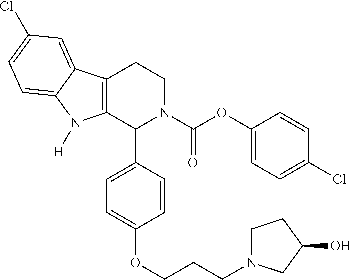 [SMILES: O=C(Oc1ccc(Cl)cc1)N1CCc2c([nH]c3ccc(Cl)cc23)C1c1ccc(OCCCN2CC[C@@H](O)C2)cc1] | 1476 |
| 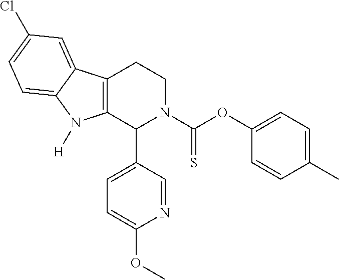 [SMILES: COc1ccc(C2c3[nH]c4ccc(Cl)cc4c3CCN2C(=S)Oc2ccc(C)cc2)cn1] | 1477 |
| 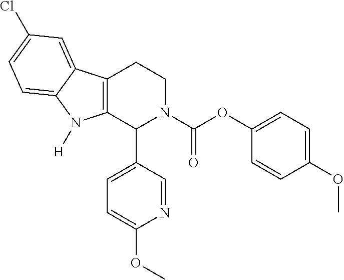 [SMILES: COc1ccc(OC(=O)N2CCc3c([nH]c4ccc(Cl)cc34)C2c2ccc(OC)nc2)cc1] | 1478 |
| 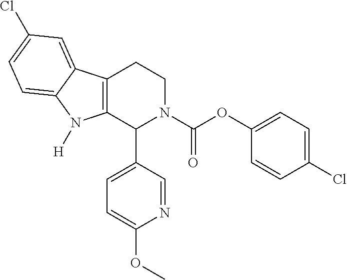 [SMILES: COc1ccc(C2c3[nH]c4ccc(Cl)cc4c3CCN2C(=O)Oc2ccc(Cl)cc2)cn1] | 1479 |
| 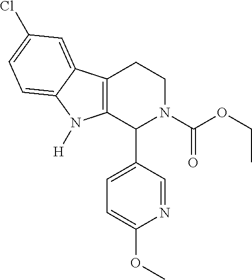 [SMILES: CCOC(=O)N1CCc2c([nH]c3ccc(Cl)cc23)C1c1ccc(OC)nc1] | 1480 |
| 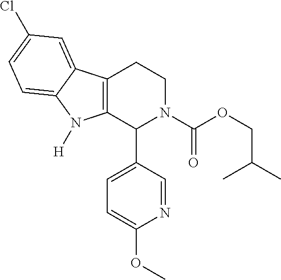 [SMILES: COc1ccc(C2c3[nH]c4ccc(Cl)cc4c3CCN2C(=O)OCC(C)C)cn1] | 1481 |
| 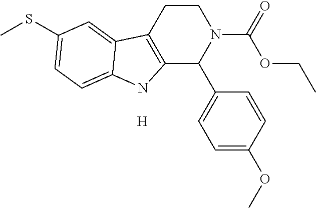 | 1482 |
| 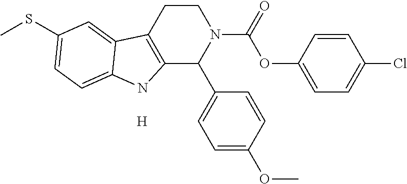 | 1483 |
| 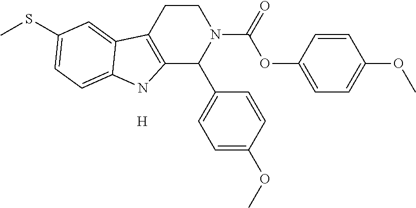 | 1484 |
| 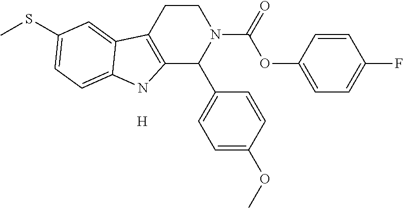 | 1485 |
| 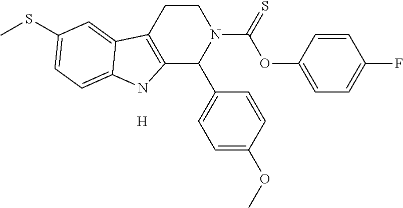 | 1486 |
| 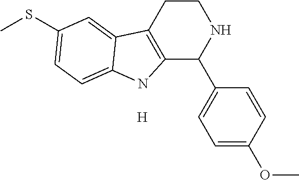 | 1487 |
| 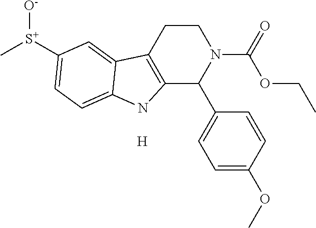 | 1488 |
| 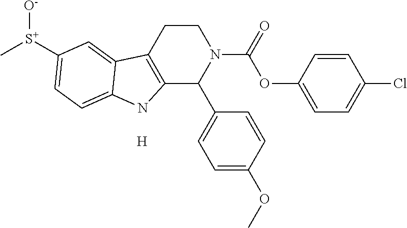 | 1489 |
| 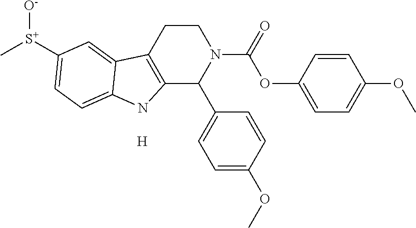 | 1490 |
| 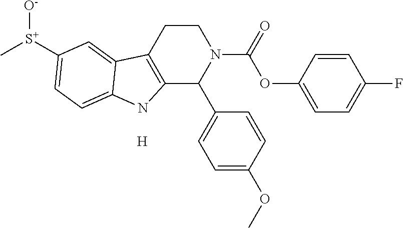 | 1491 |
| 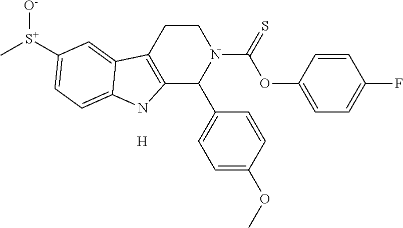 | 1492 |
| 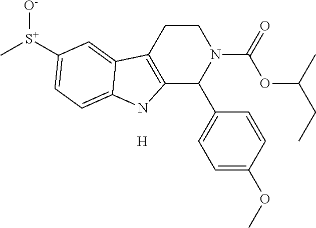 | 1493 |
| 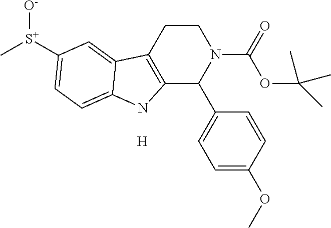 | 1494 |
| 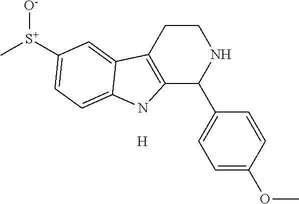 | 1495 |
| 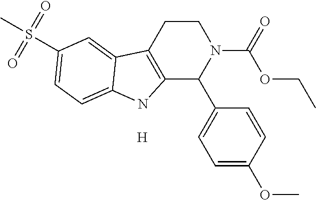 | 1496 |
| 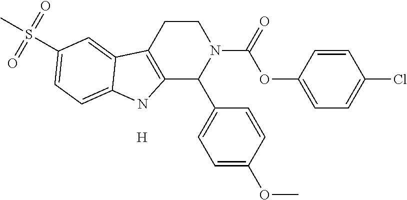 | 1497 |
| 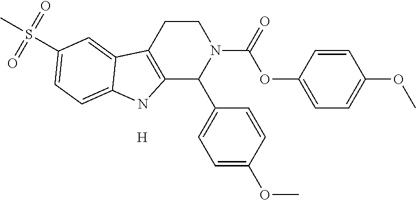 | 1498 |
| 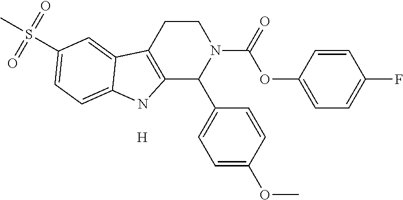 | 1499 |
| 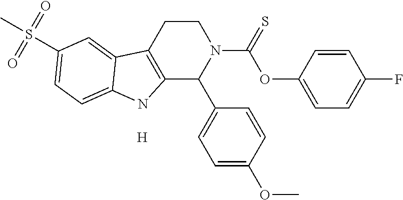 | 1500 |
| 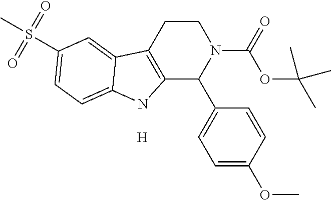 | 1502 |
| 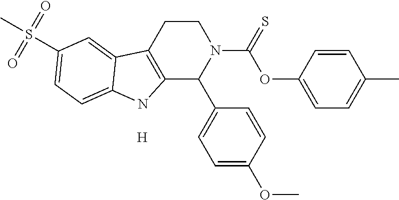 | 1503 |
| 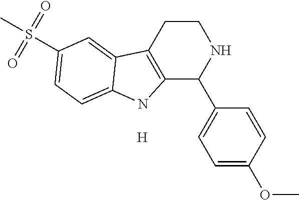 | 1504 |
| 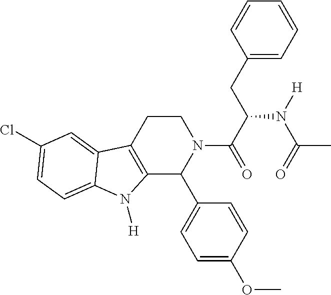 [SMILES: COc1ccc(C2c3[nH]c4ccc(Cl)cc4c3CCN2C(=O)[C@H](Cc2ccccc2)NC(C)=O)cc1] | 1505 |
| 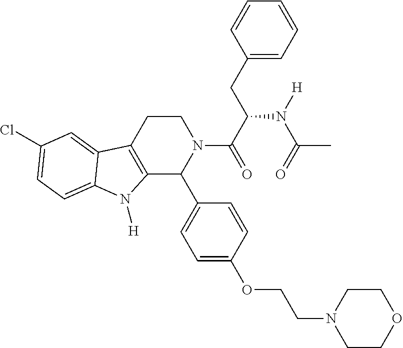 [SMILES: CC(=O)N[C@@H](Cc1ccccc1)C(=O)N1CCc2c([nH]c3ccc(Cl)cc23)C1c1ccc(OCCN2CCOCC2)cc1] | 1506 |
| 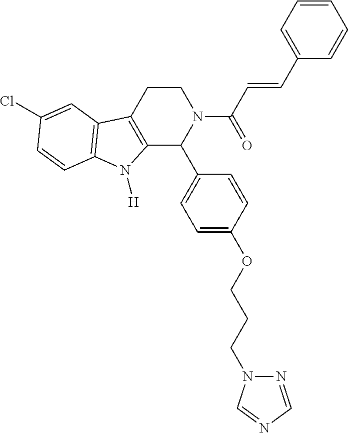 [SMILES: O=C(/C=C/c1ccccc1)N1CCc2c([nH]c3ccc(Cl)cc23)C1c1ccc(OCCCn2cncn2)cc1] | 1508 |
| 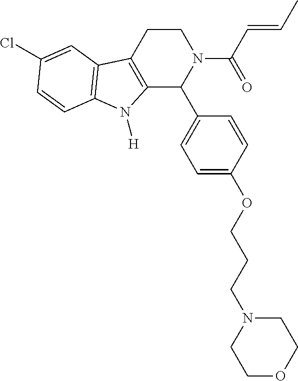 [SMILES: C/C=C/C(=O)N1CCc2c([nH]c3ccc(Cl)cc23)C1c1ccc(OCCCN2CCOCC2)cc1] | 1509 |
| 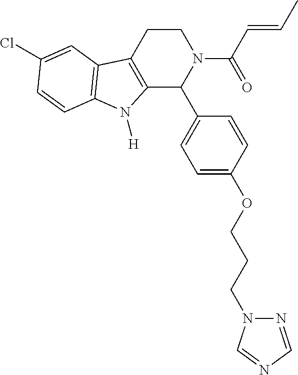 [SMILES: C/C=C/C(=O)N1CCc2c([nH]c3ccc(Cl)cc23)C1c1ccc(OCCCn2cncn2)cc1] | 1510 |
| 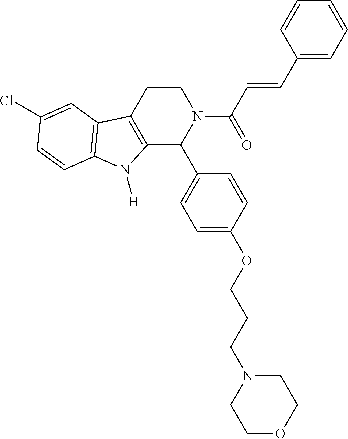 [SMILES: O=C(/C=C/c1ccccc1)N1CCc2c([nH]c3ccc(Cl)cc23)C1c1ccc(OCCCN2CCOCC2)cc1] | 1511 |
| 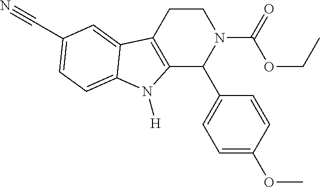 [SMILES: CCOC(=O)N1CCc2c([nH]c3ccc(C#N)cc23)C1c1ccc(OC)cc1] | 1512 |
| 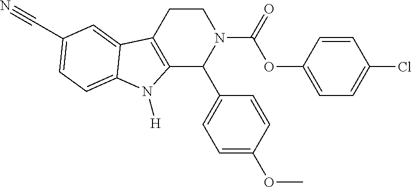 [SMILES: COc1ccc(C2c3[nH]c4ccc(C#N)cc4c3CCN2C(=O)Oc2ccc(Cl)cc2)cc1] | 1513 |
| 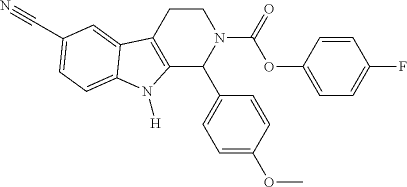 [SMILES: COc1ccc(C2c3[nH]c4ccc(C#N)cc4c3CCN2C(=O)Oc2ccc(F)cc2)cc1] | 1514 |
| 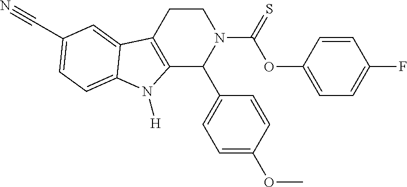 [SMILES: COc1ccc(C2c3[nH]c4ccc(C#N)cc4c3CCN2C(=S)Oc2ccc(F)cc2)cc1] | 1515 |
| 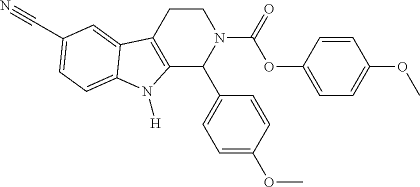 [SMILES: COc1ccc(OC(=O)N2CCc3c([nH]c4ccc(C#N)cc34)C2c2ccc(OC)cc2)cc1] | 1516 |
| 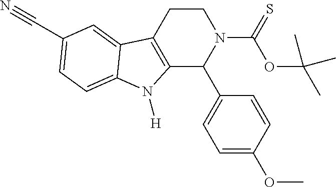 [SMILES: COc1ccc(C2c3[nH]c4ccc(C#N)cc4c3CCN2C(=S)OC(C)(C)C)cc1] | 1517 |
| 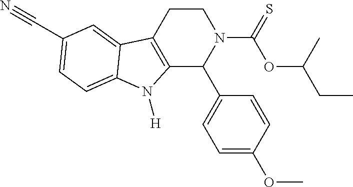 [SMILES: CCC(C)OC(=S)N1CCc2c([nH]c3ccc(C#N)cc23)C1c1ccc(OC)cc1] | 1518 |
| 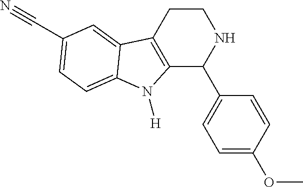 [SMILES: COc1ccc(C2NCCc3c2[nH]c2ccc(C#N)cc32)cc1] | 1519 |
| 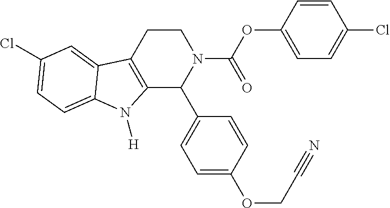 [SMILES: N#CCOc1ccc(C2c3[nH]c4ccc(Cl)cc4c3CCN2C(=O)Oc2ccc(Cl)cc2)cc1] | 1520 |
| 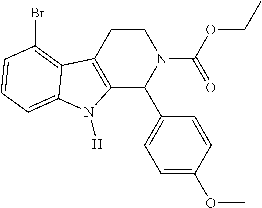 [SMILES: CCOC(=O)N1CCc2c([nH]c3cccc(Br)c23)C1c1ccc(OC)cc1] | 1521 |
| 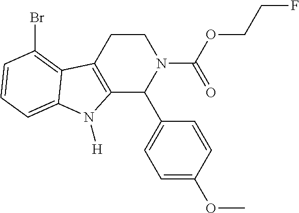 [SMILES: COc1ccc(C2c3[nH]c4cccc(Br)c4c3CCN2C(=O)OCCF)cc1] | 1522 |
| 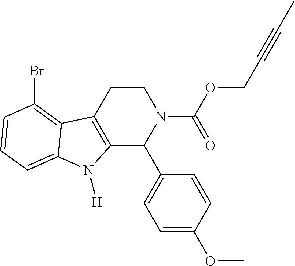 [SMILES: CC#CCOC(=O)N1CCc2c([nH]c3cccc(Br)c23)C1c1ccc(OC)cc1] | 1523 |
| 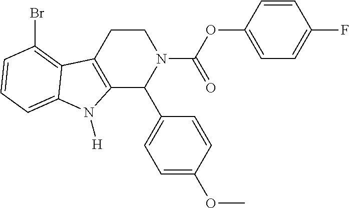 [SMILES: COc1ccc(C2c3[nH]c4cccc(Br)c4c3CCN2C(=O)Oc2ccc(F)cc2)cc1] | 1524 |
| 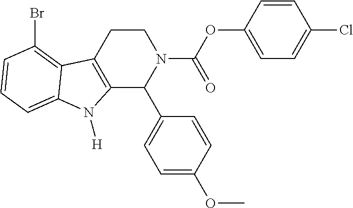 [SMILES: COc1ccc(C2c3[nH]c4cccc(Br)c4c3CCN2C(=O)Oc2ccc(Cl)cc2)cc1] | 1525 |
| 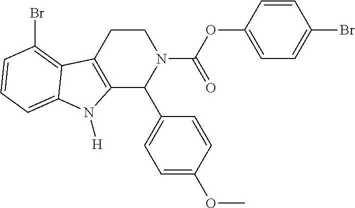 [SMILES: COc1ccc(C2c3[nH]c4cccc(Br)c4c3CCN2C(=O)Oc2ccc(Br)cc2)cc1] | 1526 |
| 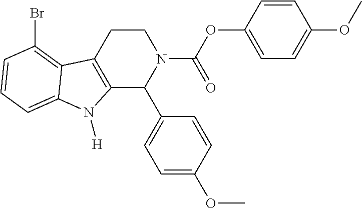 [SMILES: COc1ccc(OC(=O)N2CCc3c([nH]c4cccc(Br)c34)C2c2ccc(OC)cc2)cc1] | 1527 |
| 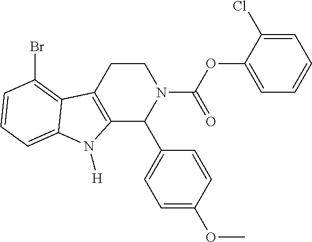 [SMILES: COc1ccc(C2c3[nH]c4cccc(Br)c4c3CCN2C(=O)Oc2ccccc2Cl)cc1] | 1528 |
| 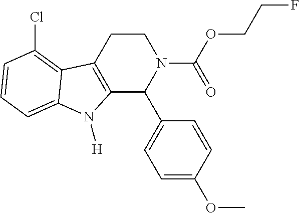 [SMILES: COc1ccc(C2c3[nH]c4cccc(Cl)c4c3CCN2C(=O)OCCF)cc1] | 1529 |
| 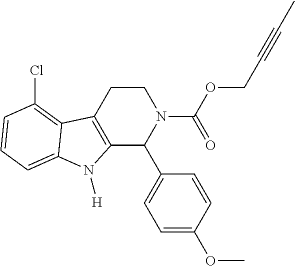 [SMILES: CC#CCOC(=O)N1CCc2c([nH]c3cccc(Cl)c23)C1c1ccc(OC)cc1] | 1530 |
| 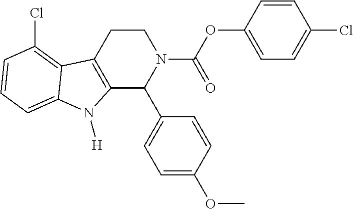 [SMILES: COc1ccc(C2c3[nH]c4cccc(Cl)c4c3CCN2C(=O)Oc2ccc(Cl)cc2)cc1] | 1531 |
| 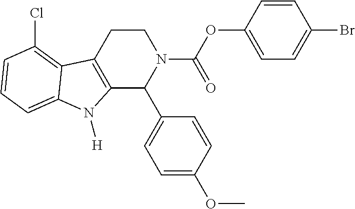 [SMILES: COc1ccc(C2c3[nH]c4cccc(Cl)c4c3CCN2C(=O)Oc2ccc(Br)cc2)cc1] | 1532 |
| 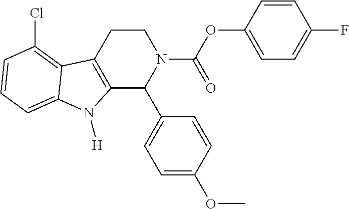 [SMILES: COc1ccc(C2c3[nH]c4cccc(Cl)c4c3CCN2C(=O)Oc2ccc(F)cc2)cc1] | 1533 |
| 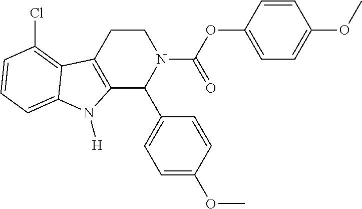 [SMILES: COc1ccc(OC(=O)N2CCc3c([nH]c4cccc(Cl)c34)C2c2ccc(OC)cc2)cc1] | 1534 |
| 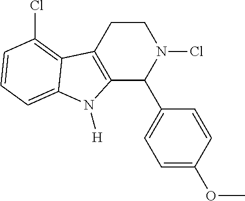 [SMILES: COc1ccc(C2c3[nH]c4cccc(Cl)c4c3CCN2Cl)cc1] | 1535 |
| 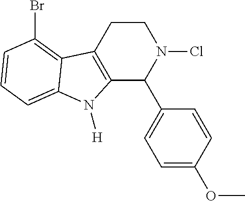 [SMILES: COc1ccc(C2c3[nH]c4cccc(Br)c4c3CCN2Cl)cc1] | 1536 |
| 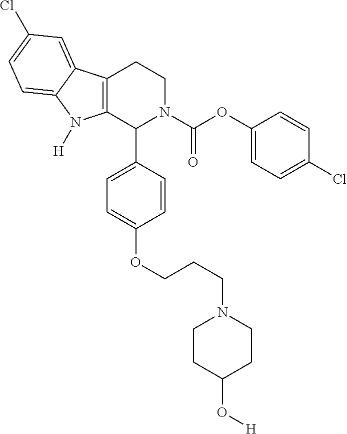 [SMILES: O=C(Oc1ccc(Cl)cc1)N1CCc2c([nH]c3ccc(Cl)cc23)C1c1ccc(OCCCN2CCC(O)CC2)cc1] | 1537 |
| 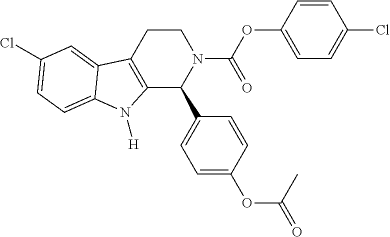 [SMILES: CC(=O)Oc1ccc([C@H]2c3[nH]c4ccc(Cl)cc4c3CCN2C(=O)Oc2ccc(Cl)cc2)cc1] | 1538 |
| 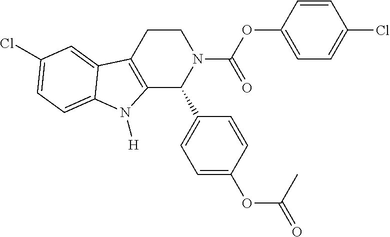 [SMILES: CC(=O)Oc1ccc([C@@H]2c3[nH]c4ccc(Cl)cc4c3CCN2C(=O)Oc2ccc(Cl)cc2)cc1] | 1539 |
| 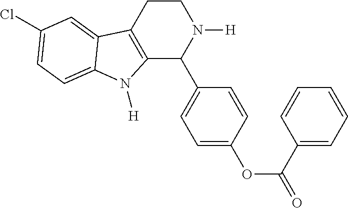 [SMILES: O=C(Oc1ccc(C2NCCc3c2[nH]c2ccc(Cl)cc32)cc1)c1ccccc1] | 1540 |
| 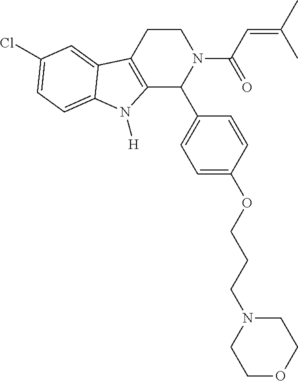 [SMILES: CC(C)=CC(=O)N1CCc2c([nH]c3ccc(Cl)cc23)C1c1ccc(OCCCN2CCOCC2)cc1] | 1541 |
| 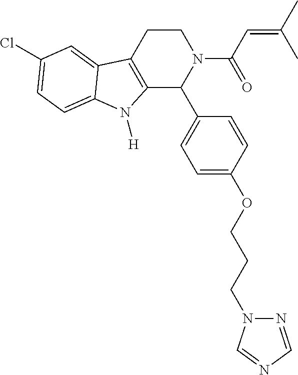 [SMILES: CC(C)=CC(=O)N1CCc2c([nH]c3ccc(Cl)cc23)C1c1ccc(OCCCn2cncn2)cc1] | 1542 |
| 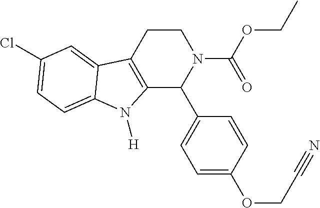 [SMILES: CCOC(=O)N1CCc2c([nH]c3ccc(Cl)cc23)C1c1ccc(OCC#N)cc1] | 1543 |
| 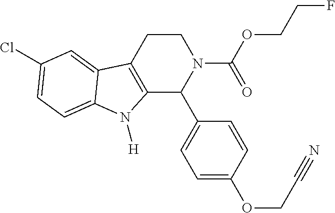 [SMILES: N#CCOc1ccc(C2c3[nH]c4ccc(Cl)cc4c3CCN2C(=O)OCCF)cc1] | 1544 |
| 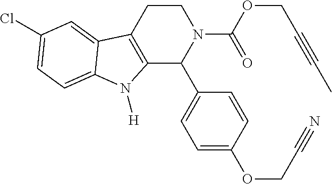 [SMILES: CC#CCOC(=O)N1CCc2c([nH]c3ccc(Cl)cc23)C1c1ccc(OCC#N)cc1] | 1545 |
| 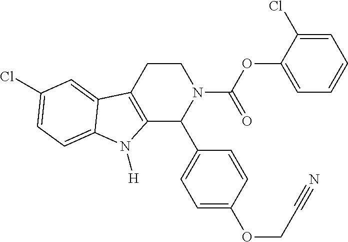 [SMILES: N#CCOc1ccc(C2c3[nH]c4ccc(Cl)cc4c3CCN2C(=O)Oc2ccccc2Cl)cc1] | 1546 |
| 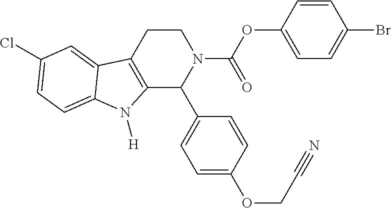 [SMILES: N#CCOc1ccc(C2c3[nH]c4ccc(Cl)cc4c3CCN2C(=O)Oc2ccc(Br)cc2)cc1] | 1547 |
| 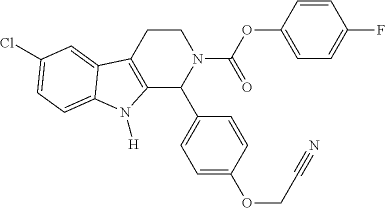 [SMILES: N#CCOc1ccc(C2c3[nH]c4ccc(Cl)cc4c3CCN2C(=O)Oc2ccc(F)cc2)cc1] | 1548 |
| 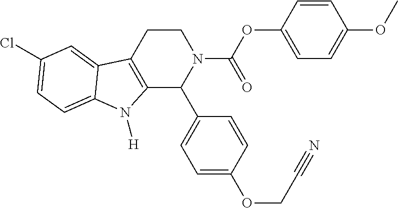 [SMILES: COc1ccc(OC(=O)N2CCc3c([nH]c4ccc(Cl)cc34)C2c2ccc(OCC#N)cc2)cc1] | 1549 |
| 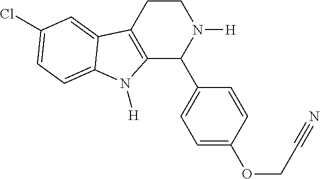 [SMILES: N#CCOc1ccc(C2NCCc3c2[nH]c2ccc(Cl)cc32)cc1] | 1550 |
| 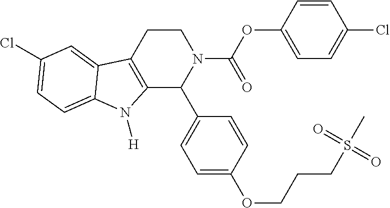 [SMILES: CS(=O)(=O)CCCOc1ccc(C2c3[nH]c4ccc(Cl)cc4c3CCN2C(=O)Oc2ccc(Cl)cc2)cc1] | 1551 |
| 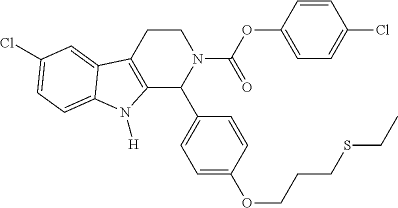 [SMILES: CCSCCCOc1ccc(C2c3[nH]c4ccc(Cl)cc4c3CCN2C(=O)Oc2ccc(Cl)cc2)cc1] | 1552 |
| 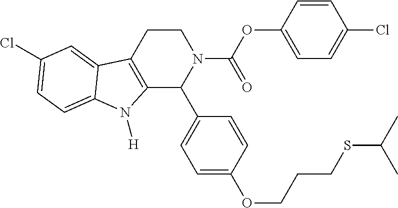 [SMILES: CC(C)SCCCOc1ccc(C2c3[nH]c4ccc(Cl)cc4c3CCN2C(=O)Oc2ccc(Cl)cc2)cc1] | 1553 |
| 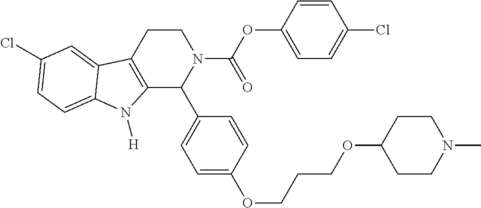 [SMILES: CN1CCC(OCCCOc2ccc(C3c4[nH]c5ccc(Cl)cc5c4CCN3C(=O)Oc3ccc(Cl)cc3)cc2)CC1] | 1554 |
| 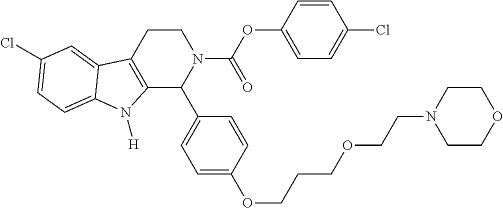 [SMILES: O=C(Oc1ccc(Cl)cc1)N1CCc2c([nH]c3ccc(Cl)cc23)C1c1ccc(OCCCOCCN2CCOCC2)cc1] | 1555 |
| 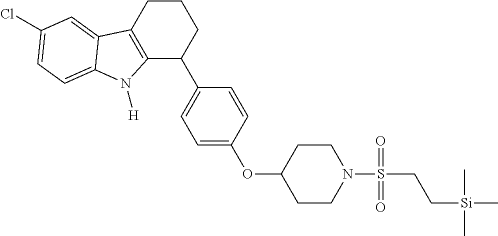 [SMILES: C[Si](C)(C)CCS(=O)(=O)N1CCC(Oc2ccc(C3CCCc4c3[nH]c3ccc(Cl)cc43)cc2)CC1] | 1556 |
| 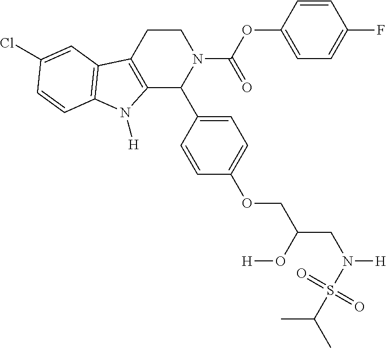 [SMILES: CC(C)S(=O)(=O)NCC(O)COc1ccc(C2c3[nH]c4ccc(Cl)cc4c3CCN2C(=O)Oc2ccc(F)cc2)cc1] | 1557 |
| 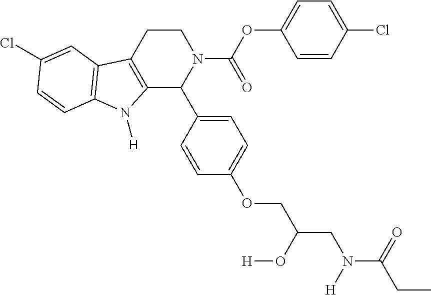 [SMILES: CCC(=O)NCC(O)COc1ccc(C2c3[nH]c4ccc(Cl)cc4c3CCN2C(=O)Oc2ccc(Cl)cc2)cc1] | 1558 |
| 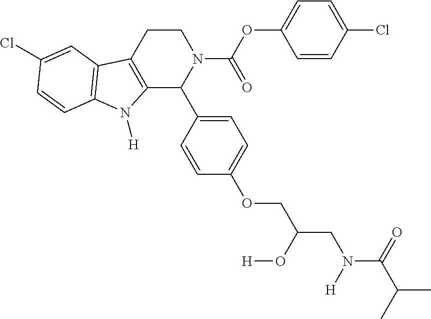 [SMILES: CC(C)C(=O)NCC(O)COc1ccc(C2c3[nH]c4ccc(Cl)cc4c3CCN2C(=O)Oc2ccc(Cl)cc2)cc1] | 1559 |
| 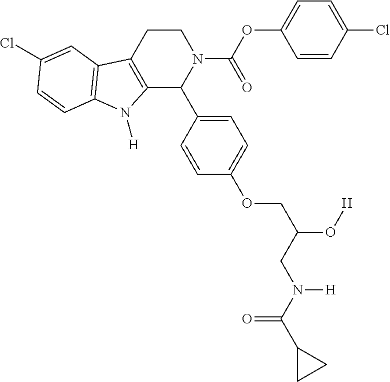 [SMILES: O=C(NCC(O)COc1ccc(C2c3[nH]c4ccc(Cl)cc4c3CCN2C(=O)Oc2ccc(Cl)cc2)cc1)C1CC1] | 1560 |
| 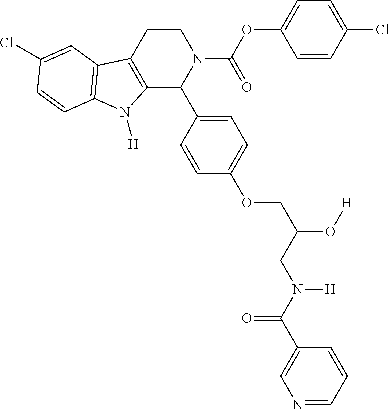 [SMILES: O=C(NCC(O)COc1ccc(C2c3[nH]c4ccc(Cl)cc4c3CCN2C(=O)Oc2ccc(Cl)cc2)cc1)c1cccnc1] | 1561 |
| 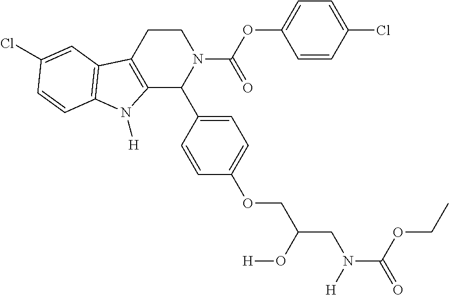 [SMILES: CCOC(=O)NCC(O)COc1ccc(C2c3[nH]c4ccc(Cl)cc4c3CCN2C(=O)Oc2ccc(Cl)cc2)cc1] | 1562 |
| 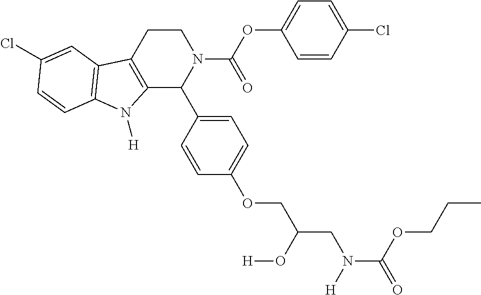 [SMILES: CCCOC(=O)NCC(O)COc1ccc(C2c3[nH]c4ccc(Cl)cc4c3CCN2C(=O)Oc2ccc(Cl)cc2)cc1] | 1563 |
| 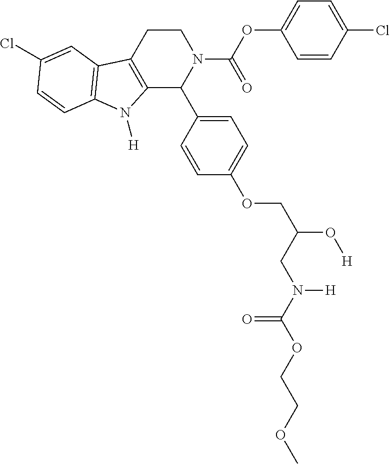 [SMILES: COCCOC(=O)NCC(O)COc1ccc(C2c3[nH]c4ccc(Cl)cc4c3CCN2C(=O)Oc2ccc(Cl)cc2)cc1] | 1564 |
| 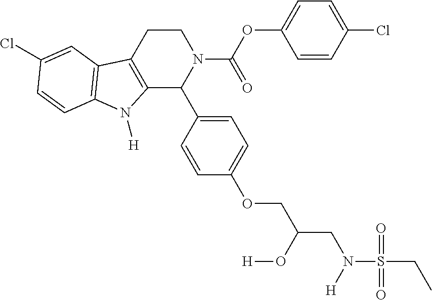 [SMILES: CCS(=O)(=O)NCC(O)COc1ccc(C2c3[nH]c4ccc(Cl)cc4c3CCN2C(=O)Oc2ccc(Cl)cc2)cc1] | 1565 |
| 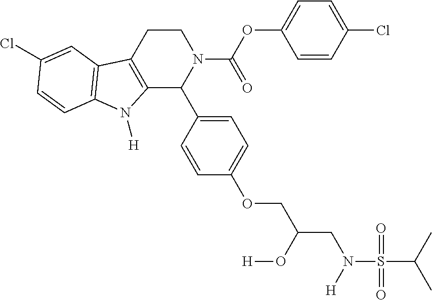 [SMILES: CC(C)S(=O)(=O)NCC(O)COc1ccc(C2c3[nH]c4ccc(Cl)cc4c3CCN2C(=O)Oc2ccc(Cl)cc2)cc1] | 1566 |
| 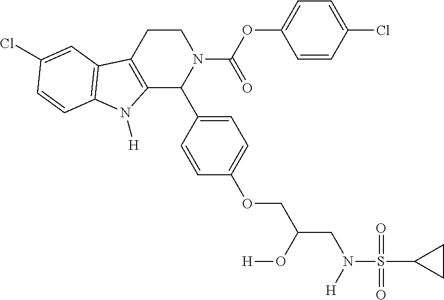 [SMILES: O=C(Oc1ccc(Cl)cc1)N1CCc2c([nH]c3ccc(Cl)cc23)C1c1ccc(OCC(O)CNS(=O)(=O)C2CC2)cc1] | 1567 |
| 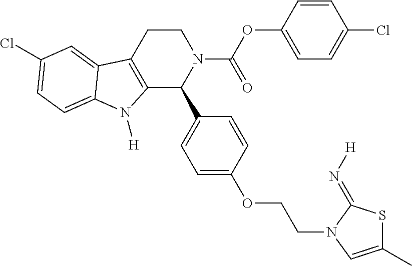 [SMILES: [H]/N=c1\sc(C)cn1CCOc1ccc([C@H]2c3[nH]c4ccc(Cl)cc4c3CCN2C(=O)Oc2ccc(Cl)cc2)cc1] | 1568 |
| 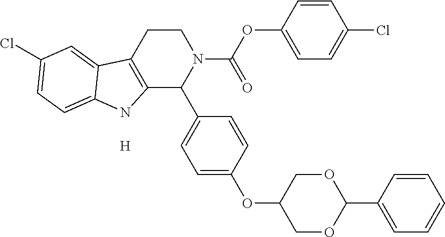 | 1569 |
| 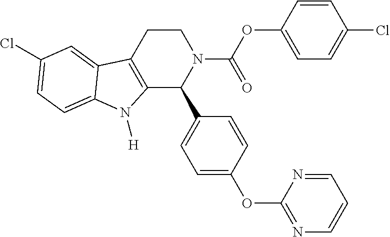 [SMILES: O=C(Oc1ccc(Cl)cc1)N1CCc2c([nH]c3ccc(Cl)cc23)[C@@H]1c1ccc(Oc2ncccn2)cc1] | 1570 |
| 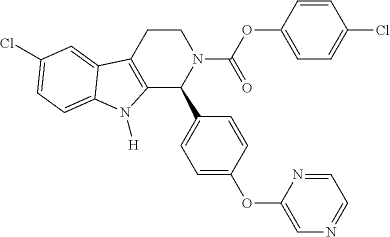 [SMILES: O=C(Oc1ccc(Cl)cc1)N1CCc2c([nH]c3ccc(Cl)cc23)[C@@H]1c1ccc(Oc2cnccn2)cc1] | 1571 |
| 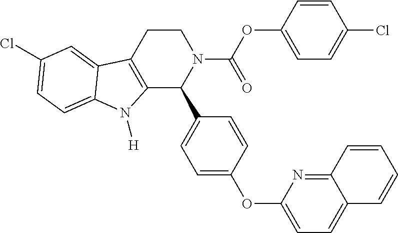 [SMILES: O=C(Oc1ccc(Cl)cc1)N1CCc2c([nH]c3ccc(Cl)cc23)[C@@H]1c1ccc(Oc2ccc3ccccc3n2)cc1] | 1572 |
| 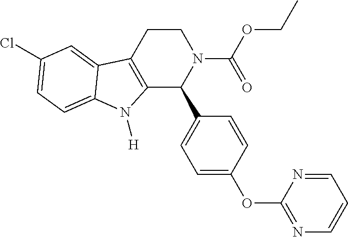 [SMILES: CCOC(=O)N1CCc2c([nH]c3ccc(Cl)cc23)[C@@H]1c1ccc(Oc2ncccn2)cc1] | 1573 |
| 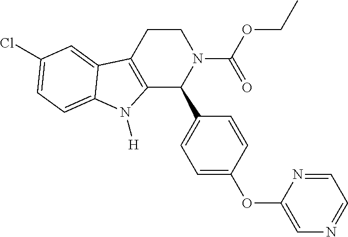 [SMILES: CCOC(=O)N1CCc2c([nH]c3ccc(Cl)cc23)[C@@H]1c1ccc(Oc2cnccn2)cc1] | 1574 |
| 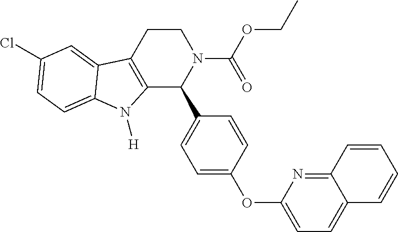 [SMILES: CCOC(=O)N1CCc2c([nH]c3ccc(Cl)cc23)[C@@H]1c1ccc(Oc2ccc3ccccc3n2)cc1] | 1575 |
| 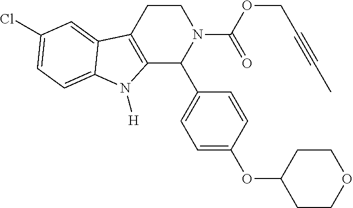 [SMILES: CC#CCOC(=O)N1CCc2c([nH]c3ccc(Cl)cc23)C1c1ccc(OC2CCOCC2)cc1] | 1576 |
| 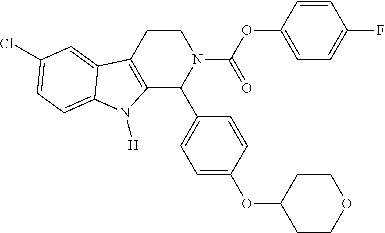 [SMILES: O=C(Oc1ccc(F)cc1)N1CCc2c([nH]c3ccc(Cl)cc23)C1c1ccc(OC2CCOCC2)cc1] | 1577 |
| 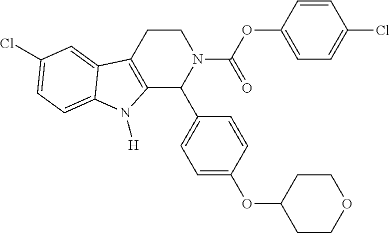 [SMILES: O=C(Oc1ccc(Cl)cc1)N1CCc2c([nH]c3ccc(Cl)cc23)C1c1ccc(OC2CCOCC2)cc1] | 1578 |
| 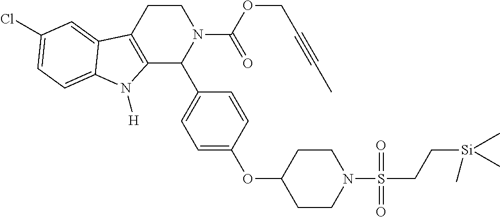 [SMILES: CC#CCOC(=O)N1CCc2c([nH]c3ccc(Cl)cc23)C1c1ccc(OC2CCN(S(=O)(=O)CC[Si](C)(C)C)CC2)cc1] | 1579 |
| 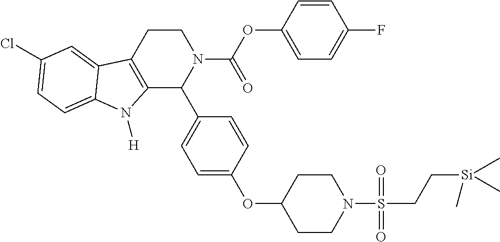 [SMILES: C[Si](C)(C)CCS(=O)(=O)N1CCC(Oc2ccc(C3c4[nH]c5ccc(Cl)cc5c4CCN3C(=O)Oc3ccc(F)cc3)cc2)CC1] | 1580 |
| 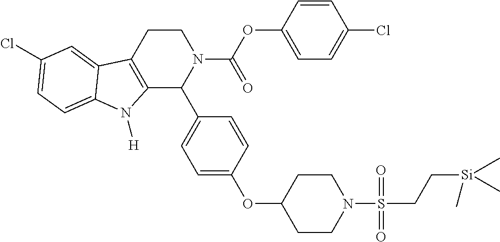 [SMILES: C[Si](C)(C)CCS(=O)(=O)N1CCC(Oc2ccc(C3c4[nH]c5ccc(Cl)cc5c4CCN3C(=O)Oc3ccc(Cl)cc3)cc2)CC1] | 1581 |
| 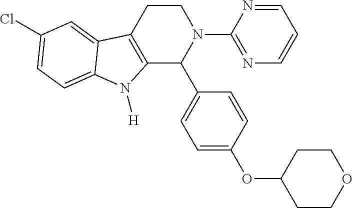 [SMILES: Clc1ccc2[nH]c3c(c2c1)CCN(c1ncccn1)C3c1ccc(OC2CCOCC2)cc1] | 1582 |
| 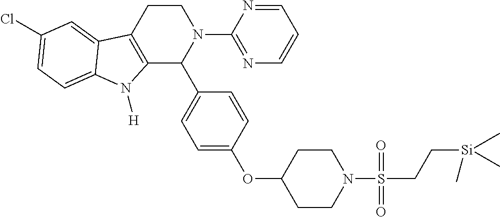 [SMILES: C[Si](C)(C)CCS(=O)(=O)N1CCC(Oc2ccc(C3c4[nH]c5ccc(Cl)cc5c4CCN3c3ncccn3)cc2)CC1] | 1583 |
| 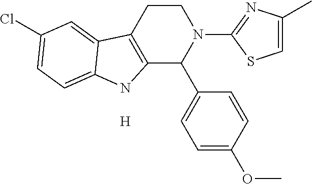 | 1584 |
| 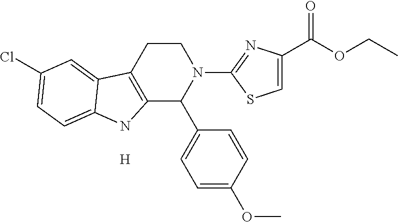 | 1585 |
| 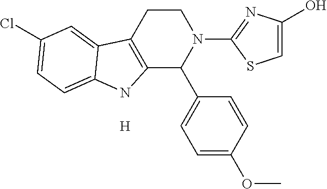 | 1586 |
| 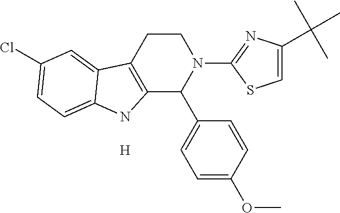 | 1587 |
| 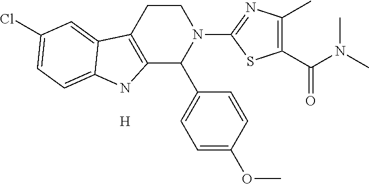 | 1588 |
| 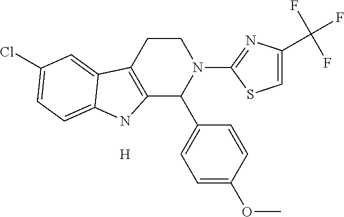 | 1589 |
| 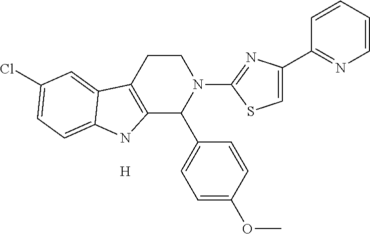 | 1590 |
| 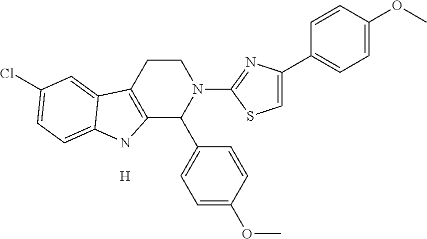 | 1591 |
| 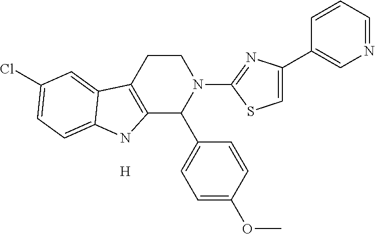 | 1592 |
| 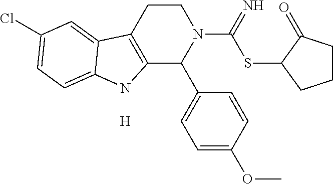 | 1593 |
| 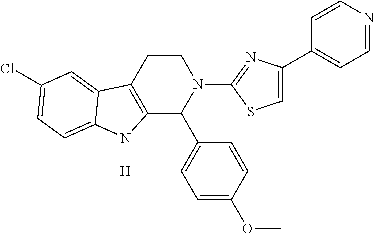 | 1594 |
| 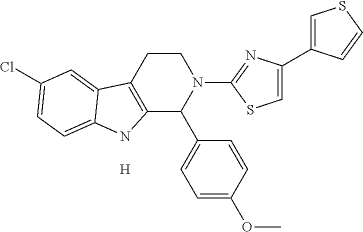 | 1595 |
| 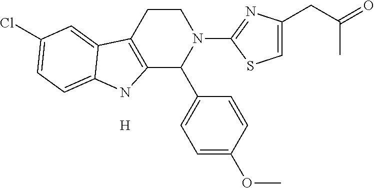 | 1596 |
| 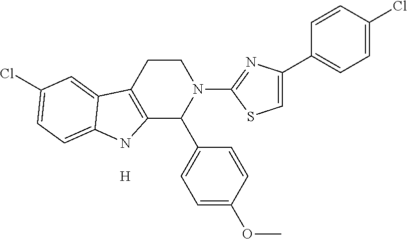 | 1597 |
| 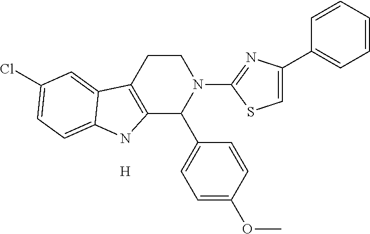 | 1598 |
| 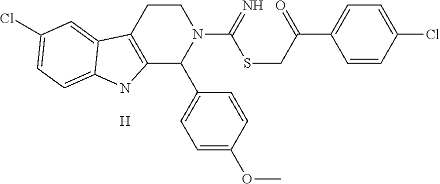 | 1599 |
| 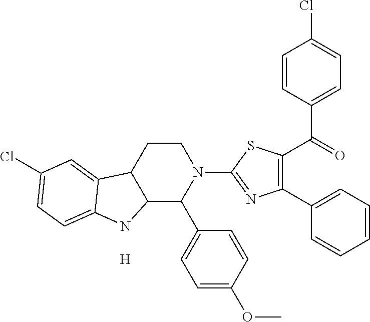 | 1600 |
| 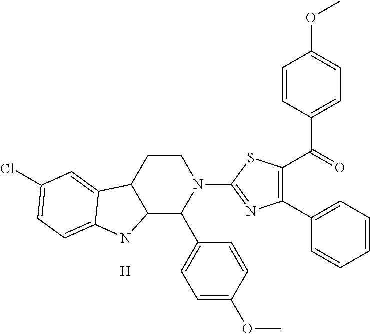 | 1601 |
| 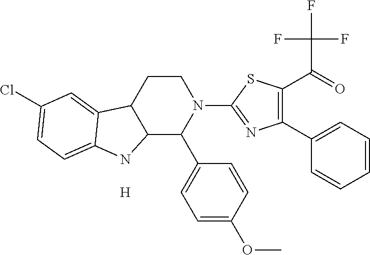 | 1602 |
| 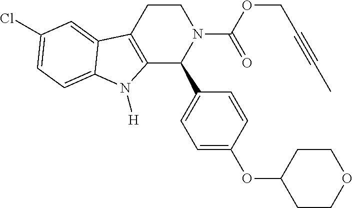 [SMILES: CC#CCOC(=O)N1CCc2c([nH]c3ccc(Cl)cc23)[C@@H]1c1ccc(OC2CCOCC2)cc1] | 1603 |
| 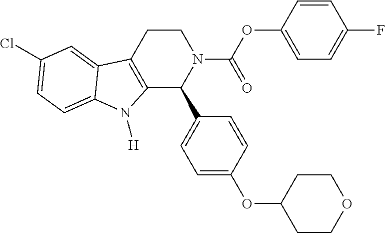 [SMILES: O=C(Oc1ccc(F)cc1)N1CCc2c([nH]c3ccc(Cl)cc23)[C@@H]1c1ccc(OC2CCOCC2)cc1] | 1604 |
| 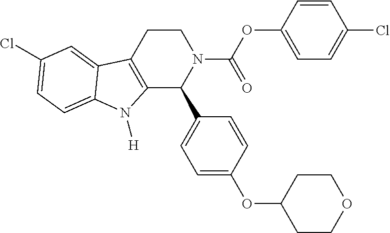 [SMILES: O=C(Oc1ccc(Cl)cc1)N1CCc2c([nH]c3ccc(Cl)cc23)[C@@H]1c1ccc(OC2CCOCC2)cc1] | 1605 |
| 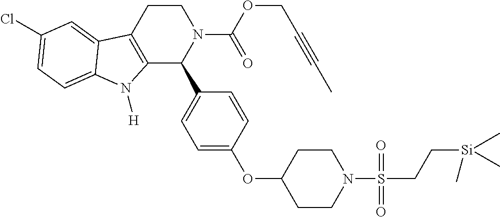 [SMILES: CC#CCOC(=O)N1CCc2c([nH]c3ccc(Cl)cc23)[C@@H]1c1ccc(OC2CCN(S(=O)(=O)CC[Si](C)(C)C)CC2)cc1] | 1606 |
| 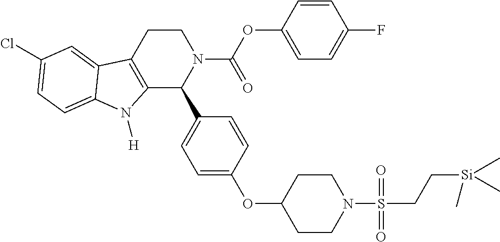 [SMILES: C[Si](C)(C)CCS(=O)(=O)N1CCC(Oc2ccc([C@H]3c4[nH]c5ccc(Cl)cc5c4CCN3C(=O)Oc3ccc(F)cc3)cc2)CC1] | 1607 |
| 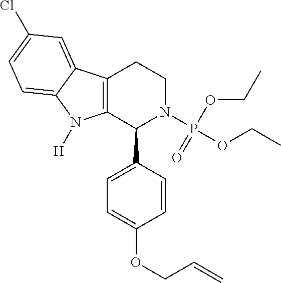 [SMILES: C=CCOc1ccc([C@H]2c3[nH]c4ccc(Cl)cc4c3CCN2P(=O)(OCC)OCC)cc1] | 1608 |
| 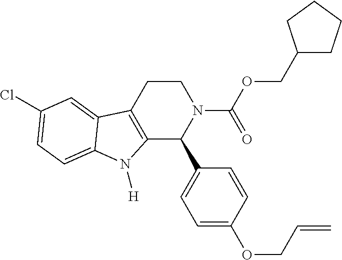 [SMILES: C=CCOc1ccc([C@H]2c3[nH]c4ccc(Cl)cc4c3CCN2C(=O)OCC2CCCC2)cc1] | 1609 |
| 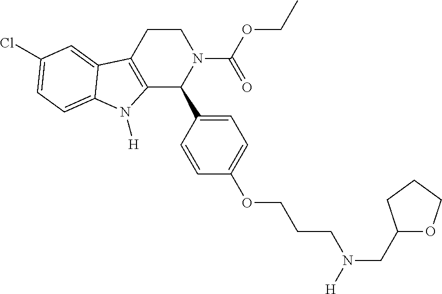 [SMILES: CCOC(=O)N1CCc2c([nH]c3ccc(Cl)cc23)[C@@H]1c1ccc(OCCCNCC2CCCO2)cc1] | 1610 |
| 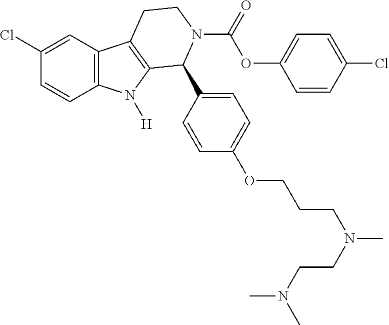 [SMILES: CN(C)CCN(C)CCCOc1ccc([C@H]2c3[nH]c4ccc(Cl)cc4c3CCN2C(=O)Oc2ccc(Cl)cc2)cc1] | 1611 |
| 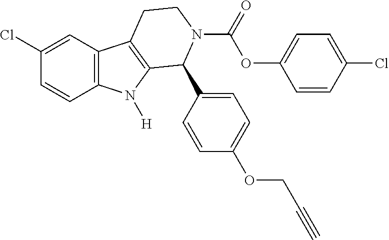 [SMILES: C#CCOc1ccc([C@H]2c3[nH]c4ccc(Cl)cc4c3CCN2C(=O)Oc2ccc(Cl)cc2)cc1] | 1612 |
| 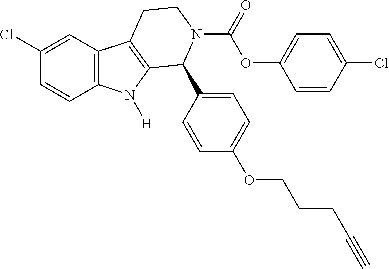 [SMILES: C#CCCCOc1ccc([C@H]2c3[nH]c4ccc(Cl)cc4c3CCN2C(=O)Oc2ccc(Cl)cc2)cc1] | 1613 |
| 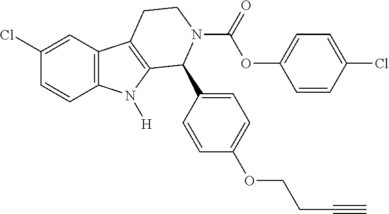 [SMILES: C#CCCOc1ccc([C@H]2c3[nH]c4ccc(Cl)cc4c3CCN2C(=O)Oc2ccc(Cl)cc2)cc1] | 1614 |
| 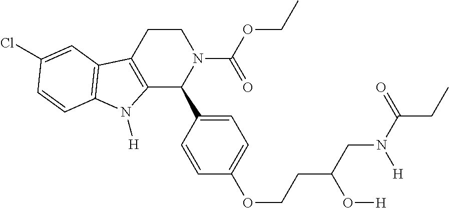 [SMILES: CCOC(=O)N1CCc2c([nH]c3ccc(Cl)cc23)[C@@H]1c1ccc(OCCC(O)CNC(=O)CC)cc1] | 1615 |
| 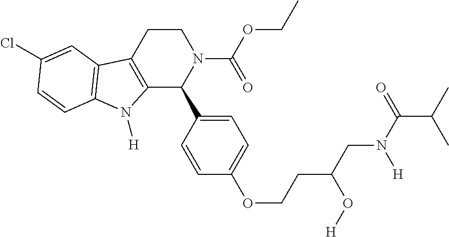 [SMILES: CCOC(=O)N1CCc2c([nH]c3ccc(Cl)cc23)[C@@H]1c1ccc(OCCC(O)CNC(=O)C(C)C)cc1] | 1616 |
| 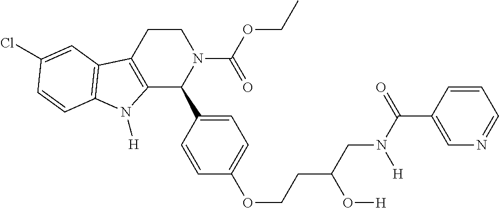 [SMILES: CCOC(=O)N1CCc2c([nH]c3ccc(Cl)cc23)[C@@H]1c1ccc(OCCC(O)CNC(=O)c2cccnc2)cc1] | 1617 |
| 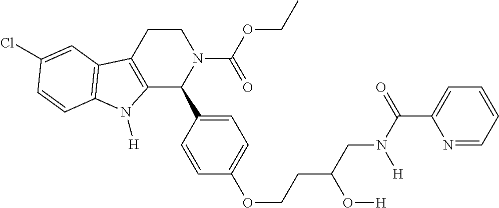 [SMILES: CCOC(=O)N1CCc2c([nH]c3ccc(Cl)cc23)[C@@H]1c1ccc(OCCC(O)CNC(=O)c2ccccn2)cc1] | 1618 |
| 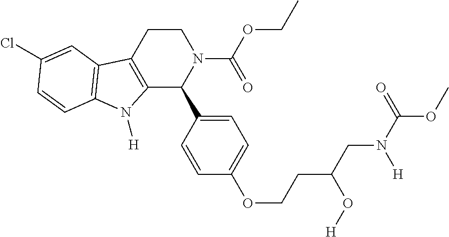 [SMILES: CCOC(=O)N1CCc2c([nH]c3ccc(Cl)cc23)[C@@H]1c1ccc(OCCC(O)CNC(=O)OC)cc1] | 1619 |
| 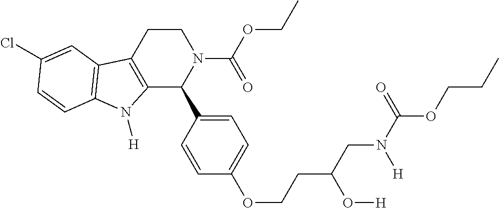 [SMILES: CCCOC(=O)NCC(O)CCOc1ccc([C@H]2c3[nH]c4ccc(Cl)cc4c3CCN2C(=O)OCC)cc1] | 1620 |
| 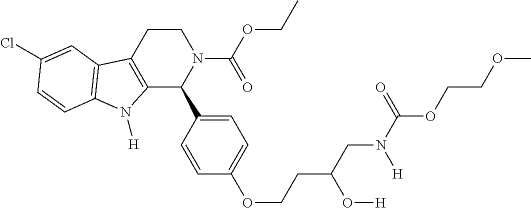 [SMILES: CCOC(=O)N1CCc2c([nH]c3ccc(Cl)cc23)[C@@H]1c1ccc(OCCC(O)CNC(=O)OCCOC)cc1] | 1621 |
| 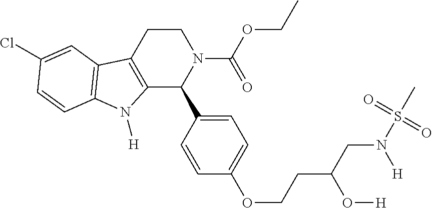 [SMILES: CCOC(=O)N1CCc2c([nH]c3ccc(Cl)cc23)[C@@H]1c1ccc(OCCC(O)CNS(C)(=O)=O)cc1] | 1622 |
| 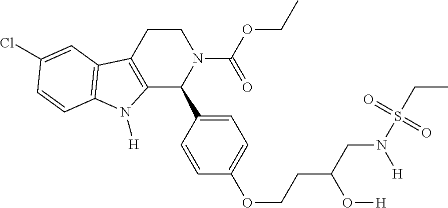 [SMILES: CCOC(=O)N1CCc2c([nH]c3ccc(Cl)cc23)[C@@H]1c1ccc(OCCC(O)CNS(=O)(=O)CC)cc1] | 1623 |
| 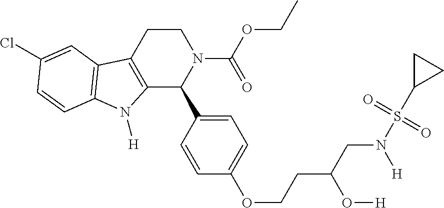 [SMILES: CCOC(=O)N1CCc2c([nH]c3ccc(Cl)cc23)[C@@H]1c1ccc(OCCC(O)CNS(=O)(=O)C2CC2)cc1] | 1624 |
| 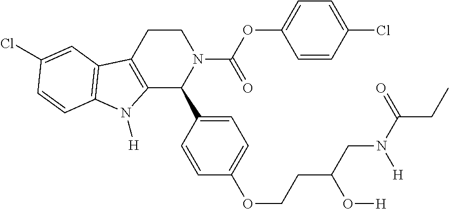 [SMILES: CCC(=O)NCC(O)CCOc1ccc([C@H]2c3[nH]c4ccc(Cl)cc4c3CCN2C(=O)Oc2ccc(Cl)cc2)cc1] | 1625 |
| 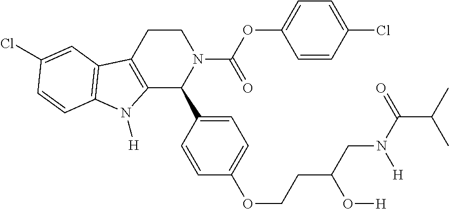 [SMILES: CC(C)C(=O)NCC(O)CCOc1ccc([C@H]2c3[nH]c4ccc(Cl)cc4c3CCN2C(=O)Oc2ccc(Cl)cc2)cc1] | 1626 |
| 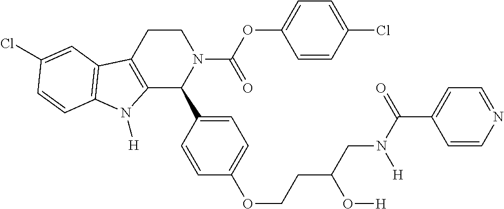 [SMILES: O=C(NCC(O)CCOc1ccc([C@H]2c3[nH]c4ccc(Cl)cc4c3CCN2C(=O)Oc2ccc(Cl)cc2)cc1)c1ccncc1] | 1627 |
| 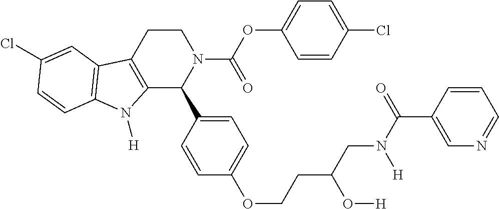 [SMILES: O=C(NCC(O)CCOc1ccc([C@H]2c3[nH]c4ccc(Cl)cc4c3CCN2C(=O)Oc2ccc(Cl)cc2)cc1)c1cccnc1] | 1628 |
| 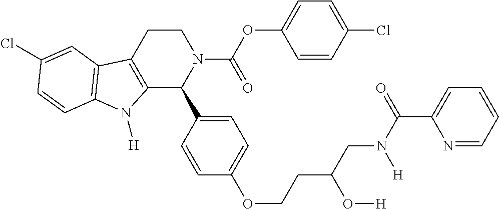 [SMILES: O=C(NCC(O)CCOc1ccc([C@H]2c3[nH]c4ccc(Cl)cc4c3CCN2C(=O)Oc2ccc(Cl)cc2)cc1)c1ccccn1] | 1629 |
| 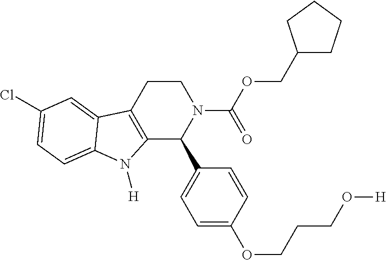 [SMILES: O=C(OCC1CCCC1)N1CCc2c([nH]c3ccc(Cl)cc23)[C@@H]1c1ccc(OCCCO)cc1] | 1630 |
| 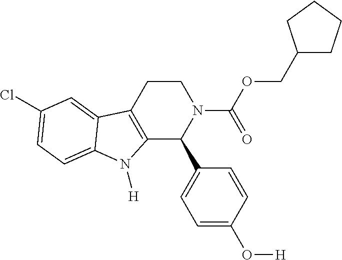 [SMILES: O=C(OCC1CCCC1)N1CCc2c([nH]c3ccc(Cl)cc23)[C@@H]1c1ccc(O)cc1] | 1631 |
| 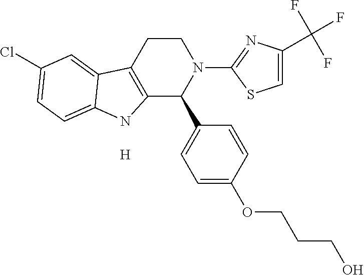 | 1632 |
| 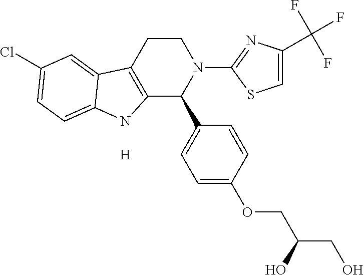 | 1633 |
| 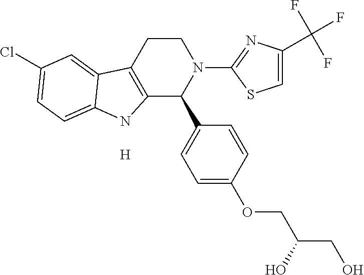 | 1634 |
| 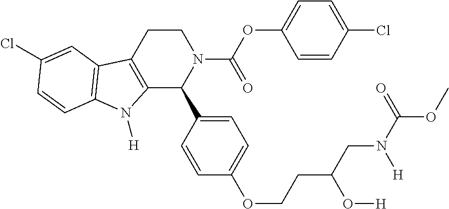 [SMILES: COC(=O)NCC(O)CCOc1ccc([C@H]2c3[nH]c4ccc(Cl)cc4c3CCN2C(=O)Oc2ccc(Cl)cc2)cc1] | 1635 |
| 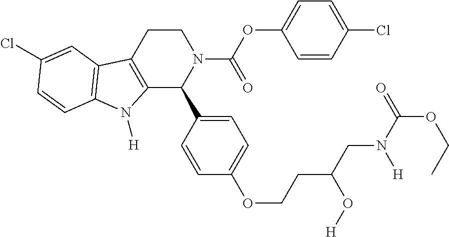 [SMILES: CCOC(=O)NCC(O)CCOc1ccc([C@H]2c3[nH]c4ccc(Cl)cc4c3CCN2C(=O)Oc2ccc(Cl)cc2)cc1] | 1636 |
| 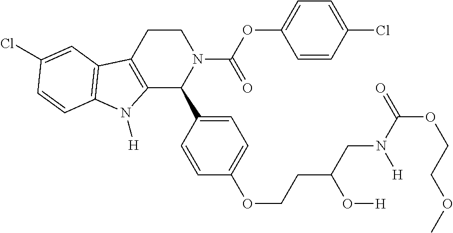 [SMILES: COCCOC(=O)NCC(O)CCOc1ccc([C@H]2c3[nH]c4ccc(Cl)cc4c3CCN2C(=O)Oc2ccc(Cl)cc2)cc1] | 1637 |
| 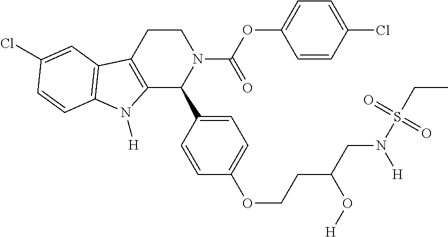 [SMILES: CCS(=O)(=O)NCC(O)CCOc1ccc([C@H]2c3[nH]c4ccc(Cl)cc4c3CCN2C(=O)Oc2ccc(Cl)cc2)cc1] | 1638 |
| 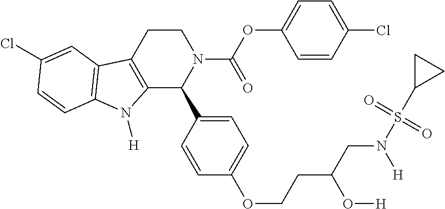 [SMILES: O=C(Oc1ccc(Cl)cc1)N1CCc2c([nH]c3ccc(Cl)cc23)[C@@H]1c1ccc(OCCC(O)CNS(=O)(=O)C2CC2)cc1] | 1639 |
| 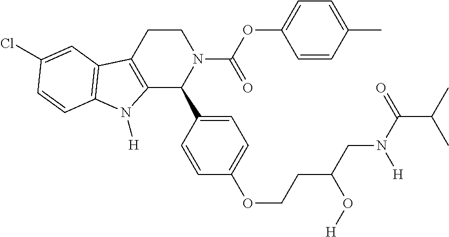 [SMILES: Cc1ccc(OC(=O)N2CCc3c([nH]c4ccc(Cl)cc34)[C@@H]2c2ccc(OCCC(O)CNC(=O)C(C)C)cc2)cc1] | 1640 |
| 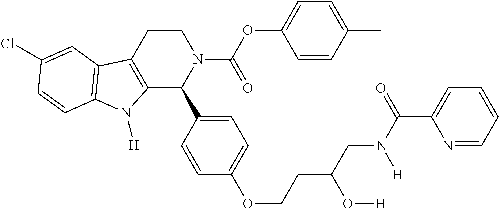 [SMILES: Cc1ccc(OC(=O)N2CCc3c([nH]c4ccc(Cl)cc34)[C@@H]2c2ccc(OCCC(O)CNC(=O)c3ccccn3)cc2)cc1] | 1641 |
| 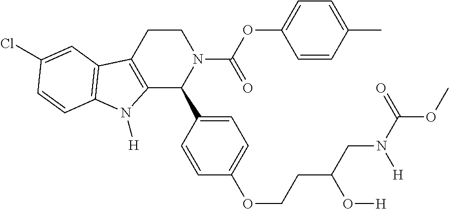 [SMILES: COC(=O)NCC(O)CCOc1ccc([C@H]2c3[nH]c4ccc(Cl)cc4c3CCN2C(=O)Oc2ccc(C)cc2)cc1] | 1642 |
| 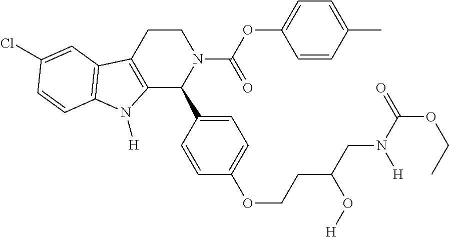 [SMILES: CCOC(=O)NCC(O)CCOc1ccc([C@H]2c3[nH]c4ccc(Cl)cc4c3CCN2C(=O)Oc2ccc(C)cc2)cc1] | 1643 |
| 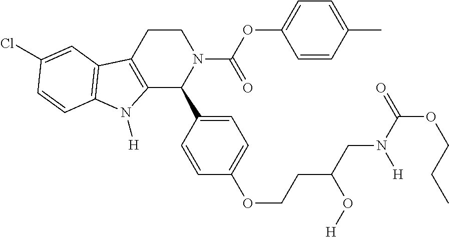 [SMILES: CCCOC(=O)NCC(O)CCOc1ccc([C@H]2c3[nH]c4ccc(Cl)cc4c3CCN2C(=O)Oc2ccc(C)cc2)cc1] | 1644 |
| 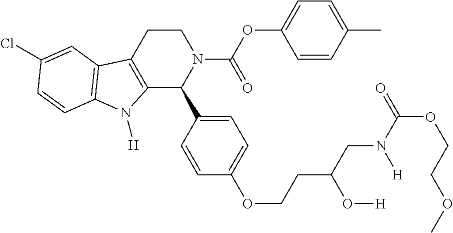 [SMILES: COCCOC(=O)NCC(O)CCOc1ccc([C@H]2c3[nH]c4ccc(Cl)cc4c3CCN2C(=O)Oc2ccc(C)cc2)cc1] | 1645 |
| 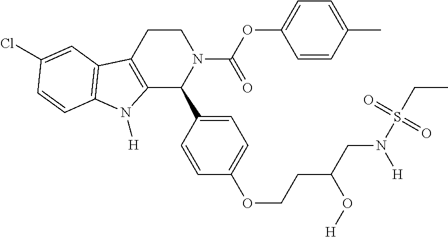 [SMILES: CCS(=O)(=O)NCC(O)CCOc1ccc([C@H]2c3[nH]c4ccc(Cl)cc4c3CCN2C(=O)Oc2ccc(C)cc2)cc1] | 1646 |
| 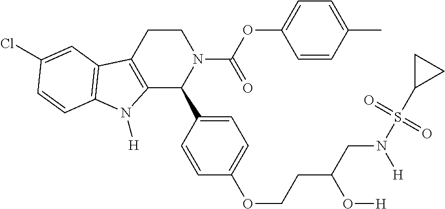 [SMILES: Cc1ccc(OC(=O)N2CCc3c([nH]c4ccc(Cl)cc34)[C@@H]2c2ccc(OCCC(O)CNS(=O)(=O)C3CC3)cc2)cc1] | 1647 |
| 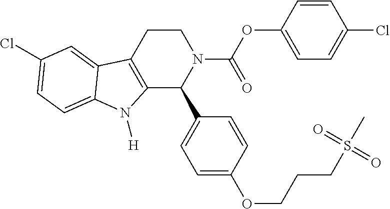 [SMILES: CS(=O)(=O)CCCOc1ccc([C@H]2c3[nH]c4ccc(Cl)cc4c3CCN2C(=O)Oc2ccc(Cl)cc2)cc1] | 1648 |
| 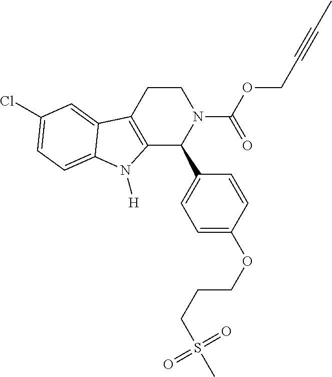 [SMILES: CC#CCOC(=O)N1CCc2c([nH]c3ccc(Cl)cc23)[C@@H]1c1ccc(OCCCS(C)(=O)=O)cc1] | 1649 |
| 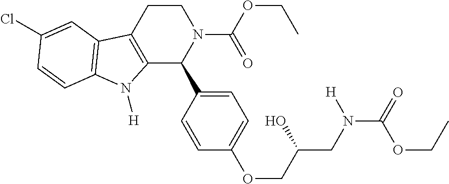 [SMILES: CCOC(=O)NC[C@@H](O)COc1ccc([C@H]2c3[nH]c4ccc(Cl)cc4c3CCN2C(=O)OCC)cc1] | 1650 |
| 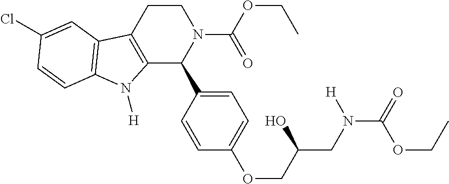 [SMILES: CCOC(=O)NC[C@H](O)COc1ccc([C@H]2c3[nH]c4ccc(Cl)cc4c3CCN2C(=O)OCC)cc1] | 1651 |
| 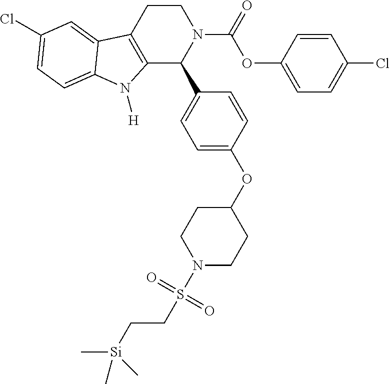 [SMILES: C[Si](C)(C)CCS(=O)(=O)N1CCC(Oc2ccc([C@H]3c4[nH]c5ccc(Cl)cc5c4CCN3C(=O)Oc3ccc(Cl)cc3)cc2)CC1] | 1652 |
| 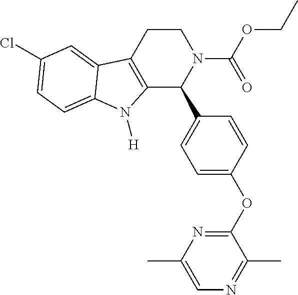 [SMILES: CCOC(=O)N1CCc2c([nH]c3ccc(Cl)cc23)[C@@H]1c1ccc(Oc2nc(C)cnc2C)cc1] | 1653 |
| 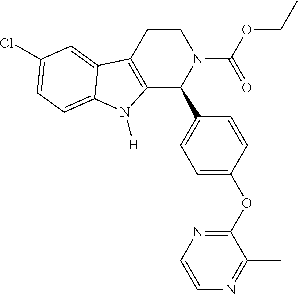 [SMILES: CCOC(=O)N1CCc2c([nH]c3ccc(Cl)cc23)[C@@H]1c1ccc(Oc2nccnc2C)cc1] | 1654 |
| 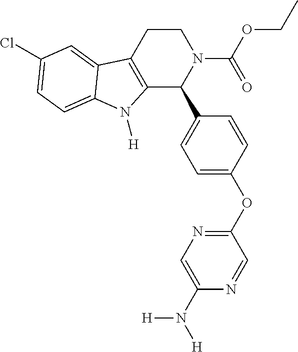 [SMILES: CCOC(=O)N1CCc2c([nH]c3ccc(Cl)cc23)[C@@H]1c1ccc(Oc2cnc(N)cn2)cc1] | 1655 |
| 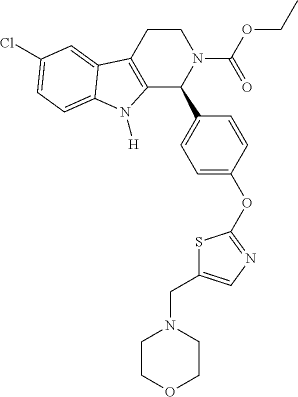 [SMILES: CCOC(=O)N1CCc2c([nH]c3ccc(Cl)cc23)[C@@H]1c1ccc(Oc2ncc(CN3CCOCC3)s2)cc1] | 1656 |
| 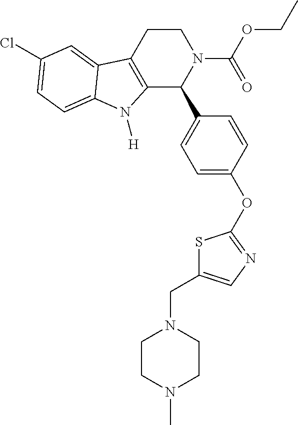 [SMILES: CCOC(=O)N1CCc2c([nH]c3ccc(Cl)cc23)[C@@H]1c1ccc(Oc2ncc(CN3CCN(C)CC3)s2)cc1] | 1657 |
| 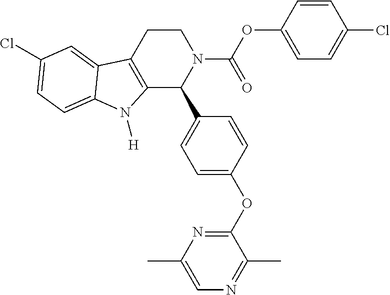 [SMILES: Cc1cnc(C)c(Oc2ccc([C@H]3c4[nH]c5ccc(Cl)cc5c4CCN3C(=O)Oc3ccc(Cl)cc3)cc2)n1] | 1658 |
| 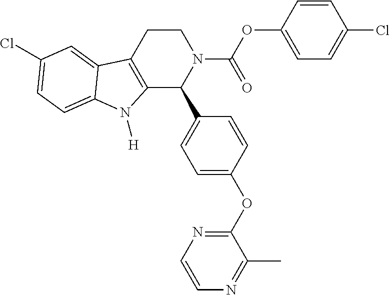 [SMILES: Cc1nccnc1Oc1ccc([C@H]2c3[nH]c4ccc(Cl)cc4c3CCN2C(=O)Oc2ccc(Cl)cc2)cc1] | 1659 |
| 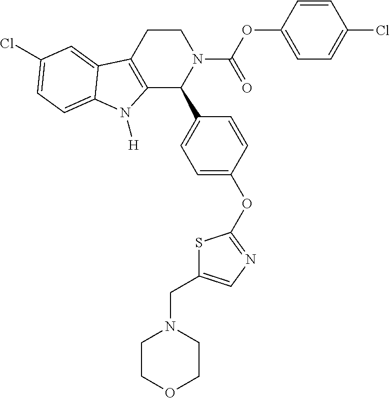 [SMILES: O=C(Oc1ccc(Cl)cc1)N1CCc2c([nH]c3ccc(Cl)cc23)[C@@H]1c1ccc(Oc2ncc(CN3CCOCC3)s2)cc1] | 1660 |
| 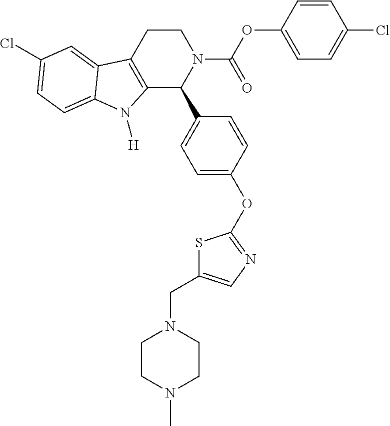 [SMILES: CN1CCN(Cc2cnc(Oc3ccc([C@H]4c5[nH]c6ccc(Cl)cc6c5CCN4C(=O)Oc4ccc(Cl)cc4)cc3)s2)CC1] | 1661 |
| 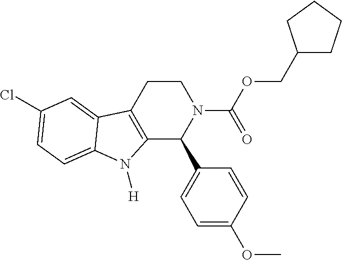 [SMILES: COc1ccc([C@H]2c3[nH]c4ccc(Cl)cc4c3CCN2C(=O)OCC2CCCC2)cc1] | 1662 |
| 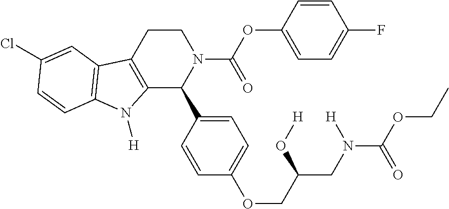 [SMILES: CCOC(=O)NC[C@H](O)COc1ccc([C@H]2c3[nH]c4ccc(Cl)cc4c3CCN2C(=O)Oc2ccc(F)cc2)cc1] | 1663 |
| 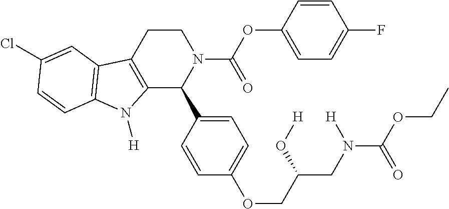 [SMILES: CCOC(=O)NC[C@@H](O)COc1ccc([C@H]2c3[nH]c4ccc(Cl)cc4c3CCN2C(=O)Oc2ccc(F)cc2)cc1] | 1664 |
| 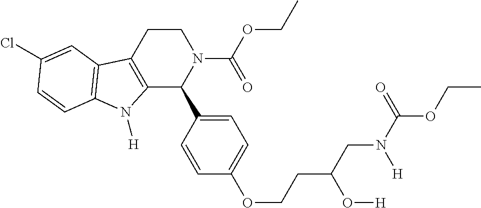 [SMILES: CCOC(=O)NCC(O)CCOc1ccc([C@H]2c3[nH]c4ccc(Cl)cc4c3CCN2C(=O)OCC)cc1] | 1665 |
| 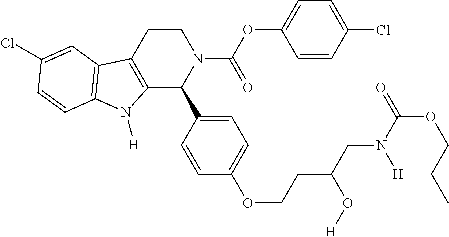 [SMILES: CCCOC(=O)NCC(O)CCOc1ccc([C@H]2c3[nH]c4ccc(Cl)cc4c3CCN2C(=O)Oc2ccc(Cl)cc2)cc1] | 1666 |
| 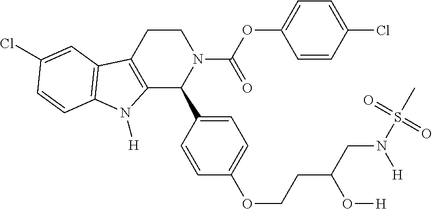 [SMILES: CS(=O)(=O)NCC(O)CCOc1ccc([C@H]2c3[nH]c4ccc(Cl)cc4c3CCN2C(=O)Oc2ccc(Cl)cc2)cc1] | 1667 |
| 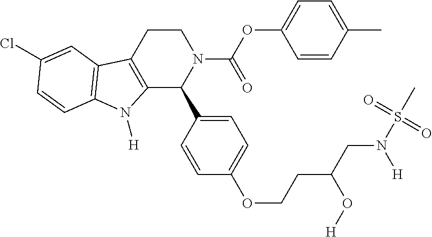 [SMILES: Cc1ccc(OC(=O)N2CCc3c([nH]c4ccc(Cl)cc34)[C@@H]2c2ccc(OCCC(O)CNS(C)(=O)=O)cc2)cc1] | 1668 |
| 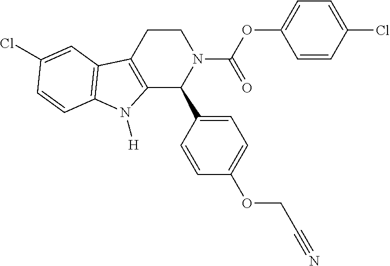 [SMILES: N#CCOc1ccc([C@H]2c3[nH]c4ccc(Cl)cc4c3CCN2C(=O)Oc2ccc(Cl)cc2)cc1] | 1669 |
| 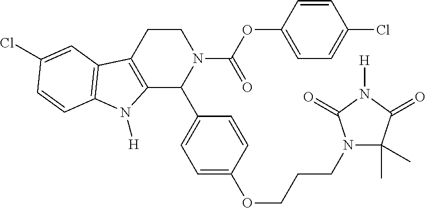 [SMILES: CC1(C)C(=O)NC(=O)N1CCCOc1ccc(C2c3[nH]c4ccc(Cl)cc4c3CCN2C(=O)Oc2ccc(Cl)cc2)cc1] | 1670 |
| 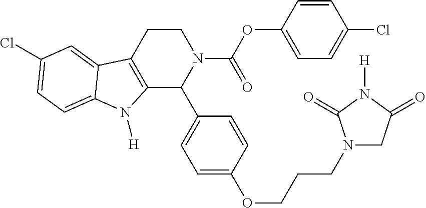 [SMILES: O=C1CN(CCCOc2ccc(C3c4[nH]c5ccc(Cl)cc5c4CCN3C(=O)Oc3ccc(Cl)cc3)cc2)C(=O)N1] | 1671 |
| 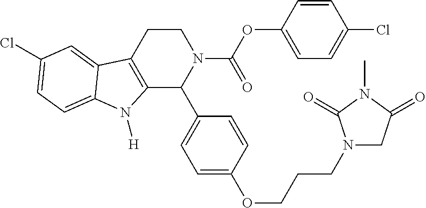 [SMILES: CN1C(=O)CN(CCCOc2ccc(C3c4[nH]c5ccc(Cl)cc5c4CCN3C(=O)Oc3ccc(Cl)cc3)cc2)C1=O] | 1672 |
| 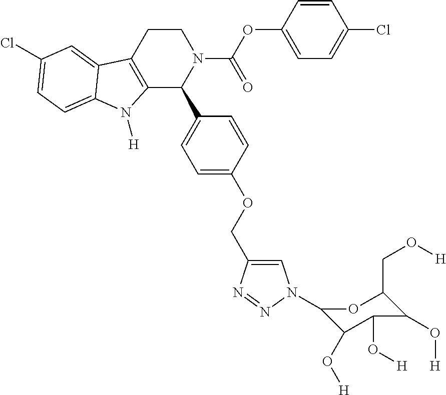 [SMILES: O=C(Oc1ccc(Cl)cc1)N1CCc2c([nH]c3ccc(Cl)cc23)[C@@H]1c1ccc(OCc2cn(C3OC(CO)C(O)C(O)C3O)nn2)cc1] | 1673 |
| 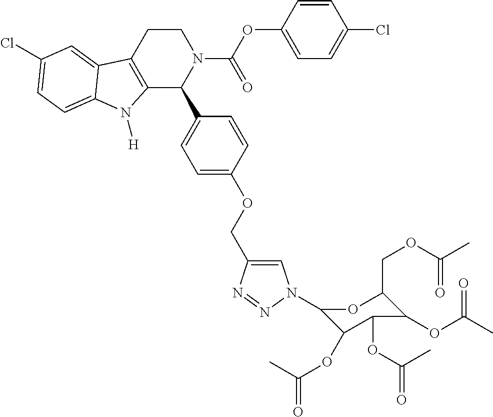 [SMILES: CC(=O)OCC1OC(n2cc(COc3ccc([C@H]4c5[nH]c6ccc(Cl)cc6c5CCN4C(=O)Oc4ccc(Cl)cc4)cc3)nn2)C(OC(C)=O)C(OC(C)=O)C1OC(C)=O] | 1674 |
| 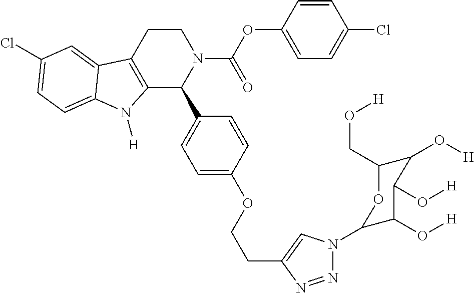 [SMILES: O=C(Oc1ccc(Cl)cc1)N1CCc2c([nH]c3ccc(Cl)cc23)[C@@H]1c1ccc(OCCc2cn(C3OC(CO)C(O)C(O)C3O)nn2)cc1] | 1675 |
| 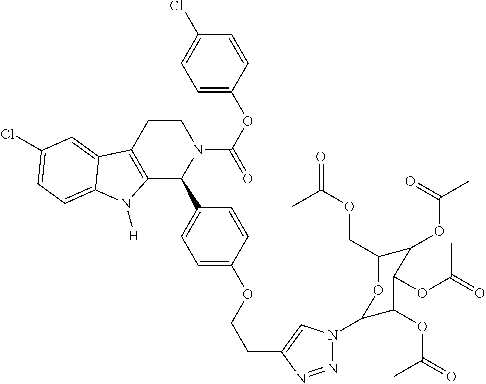 [SMILES: CC(=O)OCC1OC(n2cc(CCOc3ccc([C@H]4c5[nH]c6ccc(Cl)cc6c5CCN4C(=O)Oc4ccc(Cl)cc4)cc3)nn2)C(OC(C)=O)C(OC(C)=O)C1OC(C)=O] | 1676 |
| 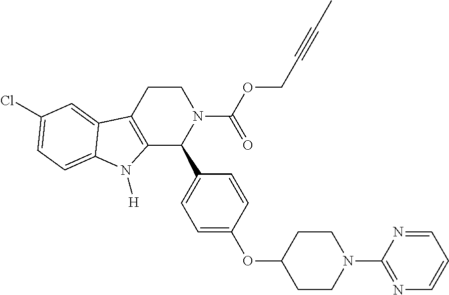 [SMILES: CC#CCOC(=O)N1CCc2c([nH]c3ccc(Cl)cc23)[C@@H]1c1ccc(OC2CCN(c3ncccn3)CC2)cc1] | 1677 |
| 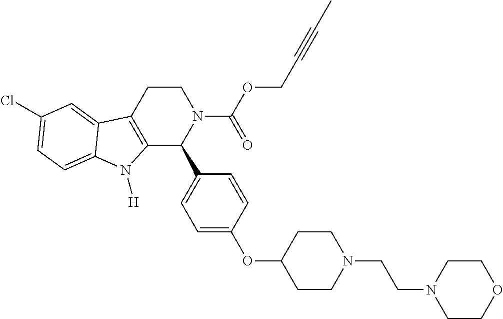 [SMILES: CC#CCOC(=O)N1CCc2c([nH]c3ccc(Cl)cc23)[C@@H]1c1ccc(OC2CCN(CCN3CCOCC3)CC2)cc1] | 1678 |
| 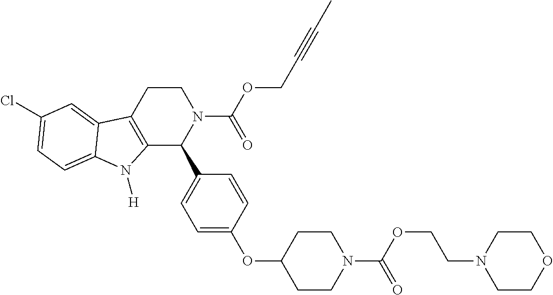 [SMILES: CC#CCOC(=O)N1CCc2c([nH]c3ccc(Cl)cc23)[C@@H]1c1ccc(OC2CCN(C(=O)OCCN3CCOCC3)CC2)cc1] | 1679 |
| 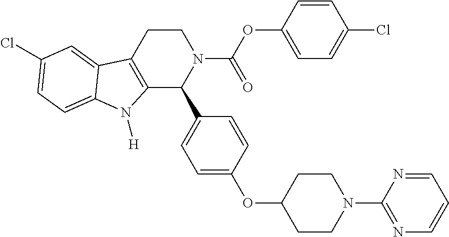 [SMILES: O=C(Oc1ccc(Cl)cc1)N1CCc2c([nH]c3ccc(Cl)cc23)[C@@H]1c1ccc(OC2CCN(c3ncccn3)CC2)cc1] | 1680 |
| 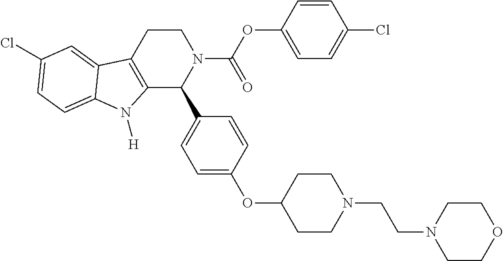 [SMILES: O=C(Oc1ccc(Cl)cc1)N1CCc2c([nH]c3ccc(Cl)cc23)[C@@H]1c1ccc(OC2CCN(CCN3CCOCC3)CC2)cc1] | 1681 |
| 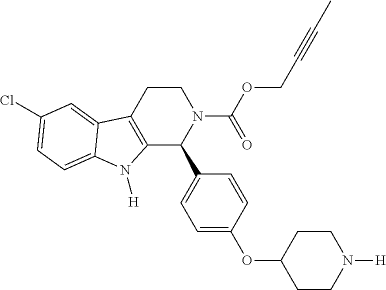 [SMILES: CC#CCOC(=O)N1CCc2c([nH]c3ccc(Cl)cc23)[C@@H]1c1ccc(OC2CCNCC2)cc1] | 1682 |
| 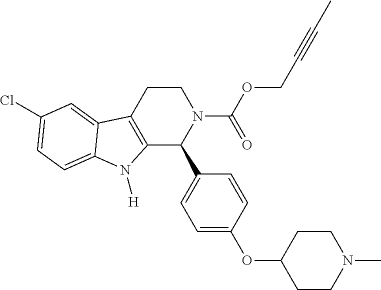 [SMILES: CC#CCOC(=O)N1CCc2c([nH]c3ccc(Cl)cc23)[C@@H]1c1ccc(OC2CCN(C)CC2)cc1] | 1683 |
| 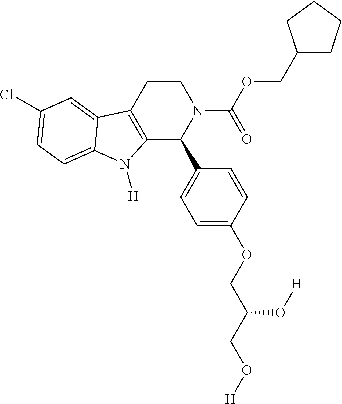 [SMILES: O=C(OCC1CCCC1)N1CCc2c([nH]c3ccc(Cl)cc23)[C@@H]1c1ccc(OC[C@H](O)CO)cc1] | 1684 |
| 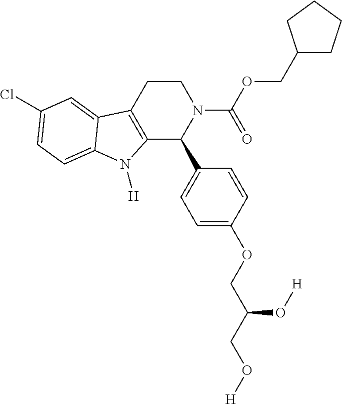 [SMILES: O=C(OCC1CCCC1)N1CCc2c([nH]c3ccc(Cl)cc23)[C@@H]1c1ccc(OC[C@@H](O)CO)cc1] | 1685 |
| 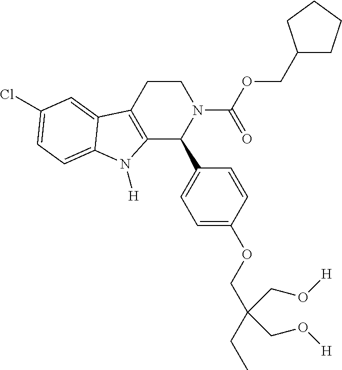 [SMILES: CCC(CO)(CO)COc1ccc([C@H]2c3[nH]c4ccc(Cl)cc4c3CCN2C(=O)OCC2CCCC2)cc1] | 1686 |
| 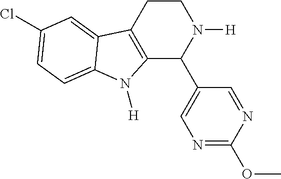 [SMILES: COc1ncc(C2NCCc3c2[nH]c2ccc(Cl)cc32)cn1] | 1687 |
| 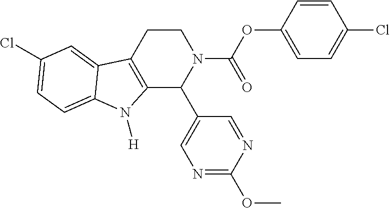 [SMILES: COc1ncc(C2c3[nH]c4ccc(Cl)cc4c3CCN2C(=O)Oc2ccc(Cl)cc2)cn1] | |
| 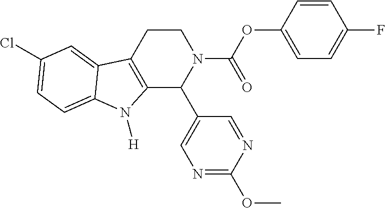 [SMILES: COc1ncc(C2c3[nH]c4ccc(Cl)cc4c3CCN2C(=O)Oc2ccc(F)cc2)cn1] | 1689 |
| 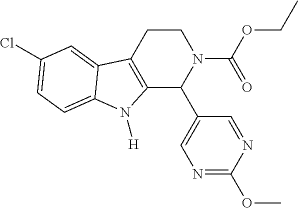 [SMILES: CCOC(=O)N1CCc2c([nH]c3ccc(Cl)cc23)C1c1cnc(OC)nc1] | 1690 |
| 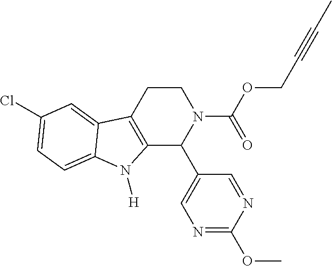 [SMILES: CC#CCOC(=O)N1CCc2c([nH]c3ccc(Cl)cc23)C1c1cnc(OC)nc1] | 1691 |
| 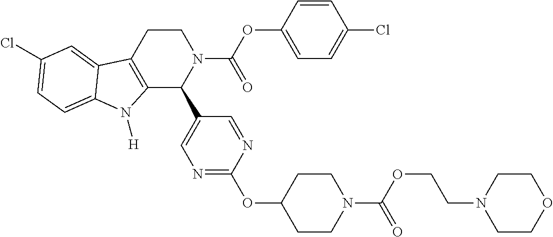 [SMILES: O=C(OCCN1CCOCC1)N1CCC(Oc2ncc([C@H]3c4[nH]c5ccc(Cl)cc5c4CCN3C(=O)Oc3ccc(Cl)cc3)cn2)CC1] | 1692 |
| 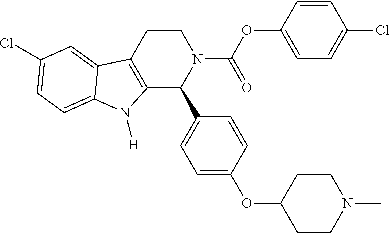 [SMILES: CN1CCC(Oc2ccc([C@H]3c4[nH]c5ccc(Cl)cc5c4CCN3C(=O)Oc3ccc(Cl)cc3)cc2)CC1] | 1693 |
| 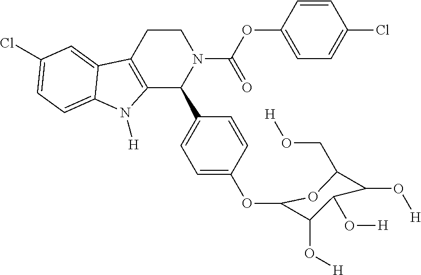 [SMILES: O=C(Oc1ccc(Cl)cc1)N1CCc2c([nH]c3ccc(Cl)cc23)[C@@H]1c1ccc(OC2OC(CO)C(O)C(O)C2O)cc1] | 1694 |
| 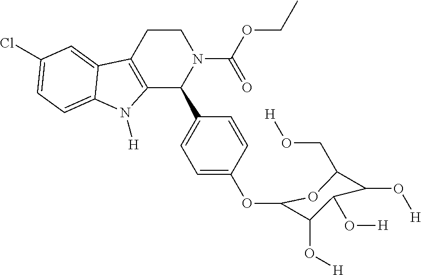 [SMILES: CCOC(=O)N1CCc2c([nH]c3ccc(Cl)cc23)[C@@H]1c1ccc(OC2OC(CO)C(O)C(O)C2O)cc1] | 1695 |
| 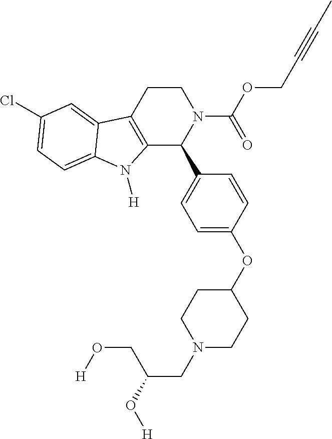 [SMILES: CC#CCOC(=O)N1CCc2c([nH]c3ccc(Cl)cc23)[C@@H]1c1ccc(OC2CCN(C[C@H](O)CO)CC2)cc1] | 1696 |
| 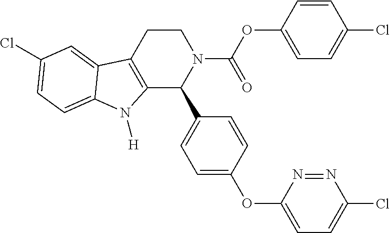 [SMILES: O=C(Oc1ccc(Cl)cc1)N1CCc2c([nH]c3ccc(Cl)cc23)[C@@H]1c1ccc(Oc2ccc(Cl)nn2)cc1] | 1697 |
| 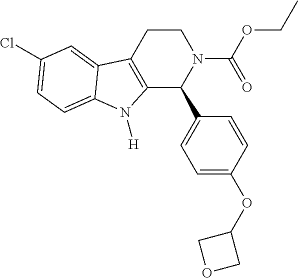 [SMILES: CCOC(=O)N1CCc2c([nH]c3ccc(Cl)cc23)[C@@H]1c1ccc(OC2COC2)cc1] | 1698 |
| 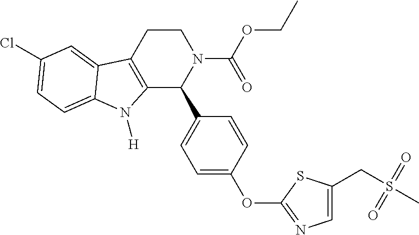 [SMILES: CCOC(=O)N1CCc2c([nH]c3ccc(Cl)cc23)[C@@H]1c1ccc(Oc2ncc(CS(C)(=O)=O)s2)cc1] | 1699 |
| 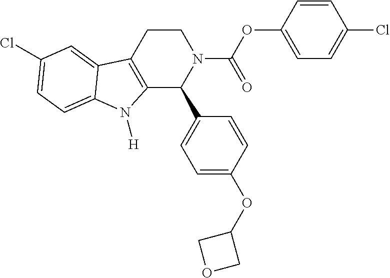 [SMILES: O=C(Oc1ccc(Cl)cc1)N1CCc2c([nH]c3ccc(Cl)cc23)[C@@H]1c1ccc(OC2COC2)cc1] | 1700 |
| 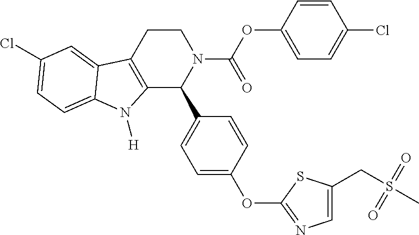 [SMILES: CS(=O)(=O)Cc1cnc(Oc2ccc([C@H]3c4[nH]c5ccc(Cl)cc5c4CCN3C(=O)Oc3ccc(Cl)cc3)cc2)s1] | 1701 |
| 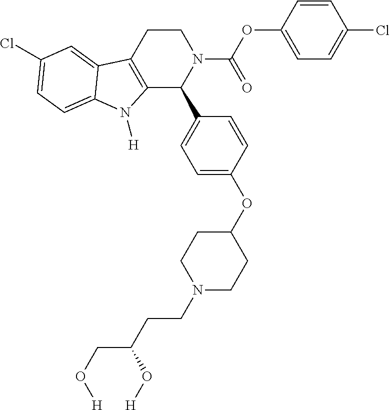 [SMILES: O=C(Oc1ccc(Cl)cc1)N1CCc2c([nH]c3ccc(Cl)cc23)[C@@H]1c1ccc(OC2CCN(CC[C@H](O)CO)CC2)cc1] | 1702 |
| 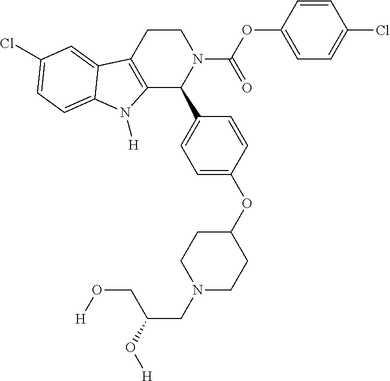 [SMILES: O=C(Oc1ccc(Cl)cc1)N1CCc2c([nH]c3ccc(Cl)cc23)[C@@H]1c1ccc(OC2CCN(C[C@H](O)CO)CC2)cc1] | 1703 |
| 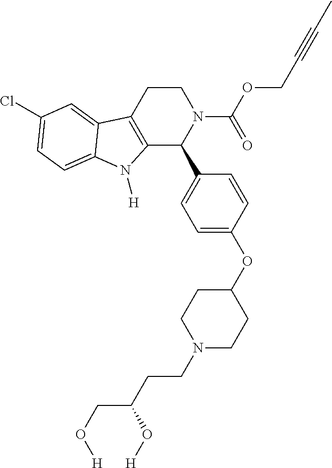 [SMILES: CC#CCOC(=O)N1CCc2c([nH]c3ccc(Cl)cc23)[C@@H]1c1ccc(OC2CCN(CC[C@H](O)CO)CC2)cc1] | 1704 |
| 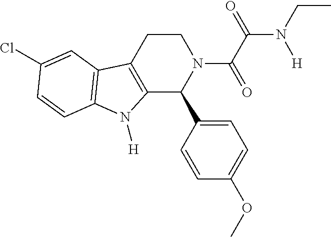 [SMILES: CCNC(=O)C(=O)N1CCc2c([nH]c3ccc(Cl)cc23)[C@@H]1c1ccc(OC)cc1] | 1705 |
| 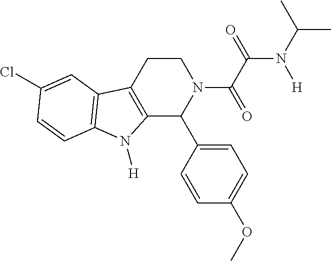 [SMILES: COc1ccc(C2c3[nH]c4ccc(Cl)cc4c3CCN2C(=O)C(=O)NC(C)C)cc1] | 1706 |
| 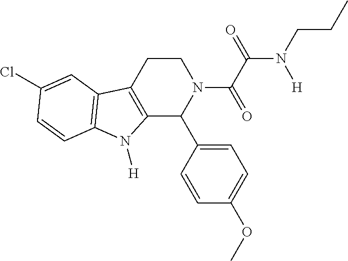 [SMILES: CCCNC(=O)C(=O)N1CCc2c([nH]c3ccc(Cl)cc23)C1c1ccc(OC)cc1] | 1707 |
| 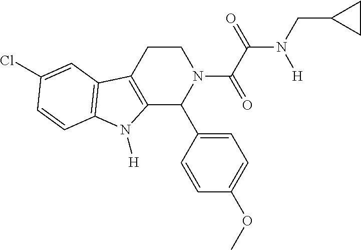 [SMILES: COc1ccc(C2c3[nH]c4ccc(Cl)cc4c3CCN2C(=O)C(=O)NCC2CC2)cc1] | 1708 |
| 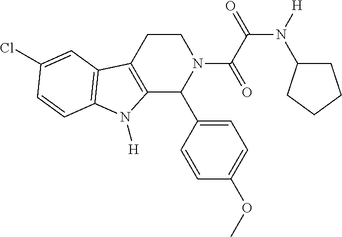 [SMILES: COc1ccc(C2c3[nH]c4ccc(Cl)cc4c3CCN2C(=O)C(=O)NC2CCCC2)cc1] | 1709 |
| 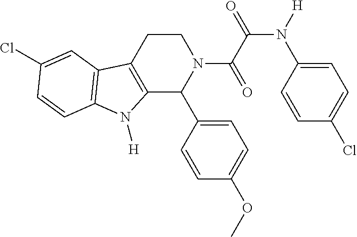 [SMILES: COc1ccc(C2c3[nH]c4ccc(Cl)cc4c3CCN2C(=O)C(=O)Nc2ccc(Cl)cc2)cc1] | 1710 |
| 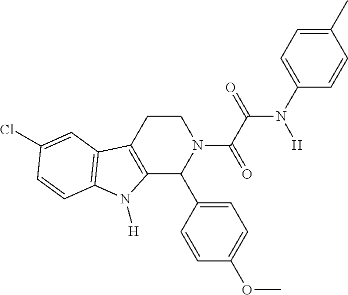 [SMILES: COc1ccc(C2c3[nH]c4ccc(Cl)cc4c3CCN2C(=O)C(=O)Nc2ccc(C)cc2)cc1] | 1711 |
| 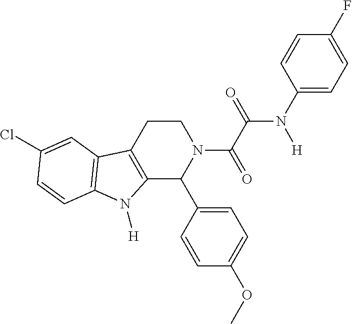 [SMILES: COc1ccc(C2c3[nH]c4ccc(Cl)cc4c3CCN2C(=O)C(=O)Nc2ccc(F)cc2)cc1] | 1712 |
| 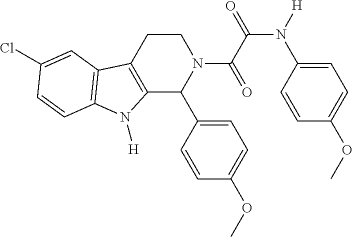 [SMILES: COc1ccc(NC(=O)C(=O)N2CCc3c([nH]c4ccc(Cl)cc34)C2c2ccc(OC)cc2)cc1] | 1713 |
| 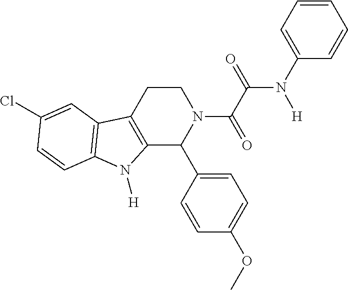 [SMILES: COc1ccc(C2c3[nH]c4ccc(Cl)cc4c3CCN2C(=O)C(=O)Nc2ccccc2)cc1] | 1714 |
| 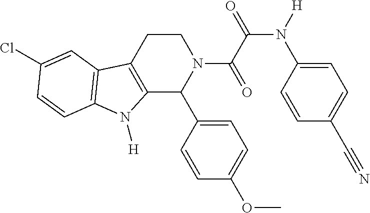 [SMILES: COc1ccc(C2c3[nH]c4ccc(Cl)cc4c3CCN2C(=O)C(=O)Nc2ccc(C#N)cc2)cc1] | 1715 |
| 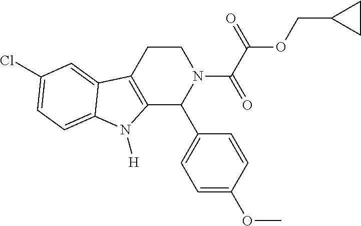 [SMILES: COc1ccc(C2c3[nH]c4ccc(Cl)cc4c3CCN2C(=O)C(=O)OCC2CC2)cc1] | 1716 |
| 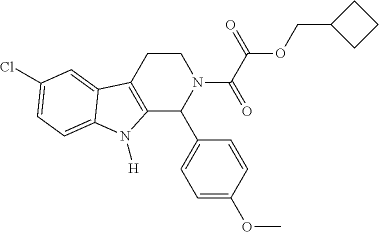 [SMILES: COc1ccc(C2c3[nH]c4ccc(Cl)cc4c3CCN2C(=O)C(=O)OCC2CCC2)cc1] | 1717 |
| 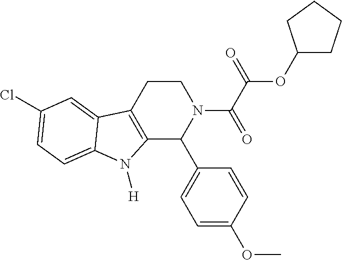 [SMILES: COc1ccc(C2c3[nH]c4ccc(Cl)cc4c3CCN2C(=O)C(=O)OC2CCCC2)cc1] | 1718 |
| 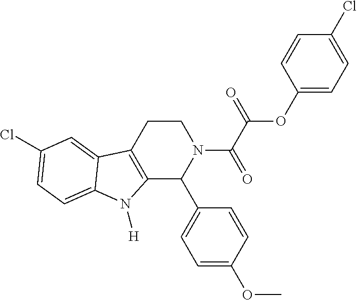 [SMILES: COc1ccc(C2c3[nH]c4ccc(Cl)cc4c3CCN2C(=O)C(=O)Oc2ccc(Cl)cc2)cc1] | 1719 |
| 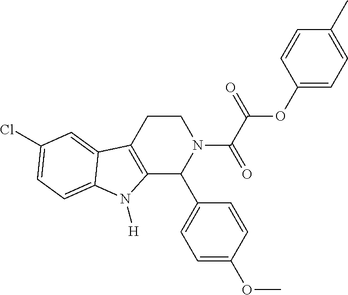 [SMILES: COc1ccc(C2c3[nH]c4ccc(Cl)cc4c3CCN2C(=O)C(=O)Oc2ccc(C)cc2)cc1] | 1720 |
| 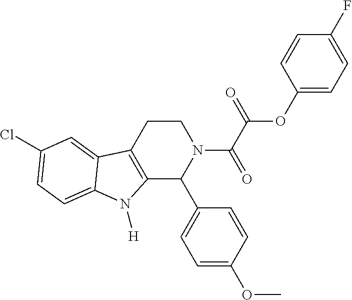 [SMILES: COc1ccc(C2c3[nH]c4ccc(Cl)cc4c3CCN2C(=O)C(=O)Oc2ccc(F)cc2)cc1] | 1721 |
| 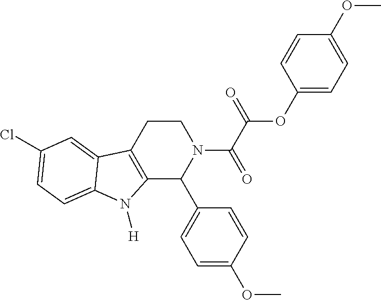 [SMILES: COc1ccc(OC(=O)C(=O)N2CCc3c([nH]c4ccc(Cl)cc34)C2c2ccc(OC)cc2)cc1] | 1722 |
| 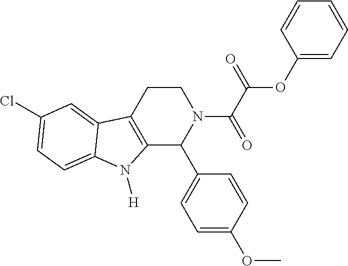 [SMILES: COc1ccc(C2c3[nH]c4ccc(Cl)cc4c3CCN2C(=O)C(=O)Oc2ccccc2)cc1] | 1723 |
| 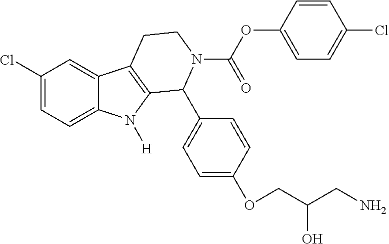 [SMILES: NCC(O)COc1ccc(C2c3[nH]c4ccc(Cl)cc4c3CCN2C(=O)Oc2ccc(Cl)cc2)cc1] | 1724 |
| 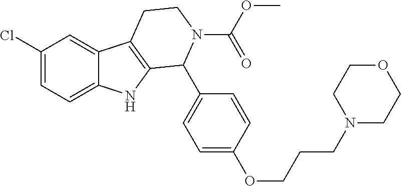 [SMILES: COC(=O)N1CCc2c([nH]c3ccc(Cl)cc23)C1c1ccc(OCCCN2CCOCC2)cc1] | 1725 |
| 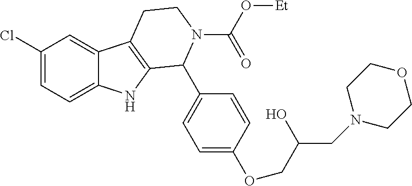 [SMILES: CCOC(=O)N1CCc2c([nH]c3ccc(Cl)cc23)C1c1ccc(OCC(O)CN2CCOCC2)cc1] | 1726 |
| 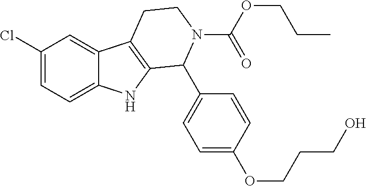 [SMILES: CCCOC(=O)N1CCc2c([nH]c3ccc(Cl)cc23)C1c1ccc(OCCCO)cc1] | 1727 |
| 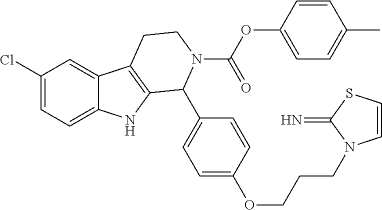 [SMILES: Cc1ccc(OC(=O)N2CCc3c([nH]c4ccc(Cl)cc34)C2c2ccc(OCCCn3ccsc3=N)cc2)cc1] | 1728 |
| 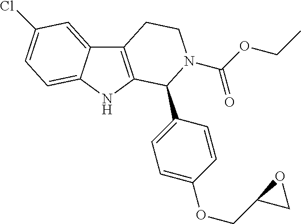 [SMILES: CCOC(=O)N1CCc2c([nH]c3ccc(Cl)cc23)[C@@H]1c1ccc(OC[C@@H]2CO2)cc1] | 1729 |
| 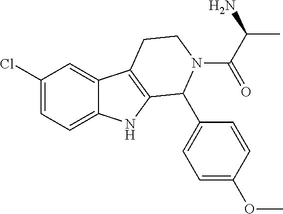 [SMILES: COc1ccc(C2c3[nH]c4ccc(Cl)cc4c3CCN2C(=O)[C@H](C)N)cc1] | 1730 |
| 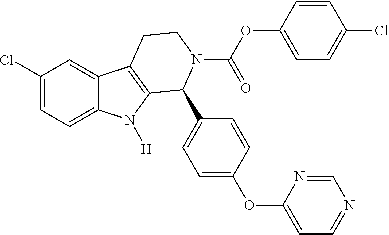 [SMILES: O=C(Oc1ccc(Cl)cc1)N1CCc2c([nH]c3ccc(Cl)cc23)[C@@H]1c1ccc(Oc2ccncn2)cc1] | 1731 |
| 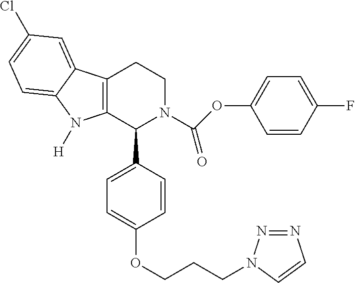 [SMILES: O=C(Oc1ccc(F)cc1)N1CCc2c([nH]c3ccc(Cl)cc23)[C@@H]1c1ccc(OCCCn2ccnn2)cc1] | 1732 |
| 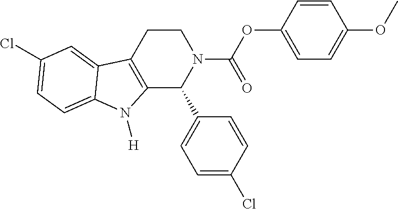 [SMILES: COc1ccc(OC(=O)N2CCc3c([nH]c4ccc(Cl)cc34)[C@H]2c2ccc(Cl)cc2)cc1] | 1733 |
| 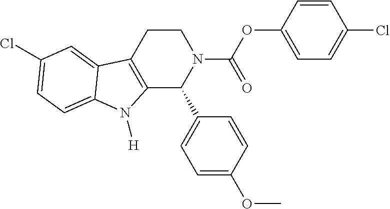 [SMILES: COc1ccc([C@@H]2c3[nH]c4ccc(Cl)cc4c3CCN2C(=O)Oc2ccc(Cl)cc2)cc1] | 1734 |
| 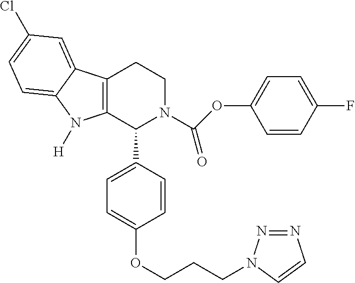 [SMILES: O=C(Oc1ccc(F)cc1)N1CCc2c([nH]c3ccc(Cl)cc23)[C@H]1c1ccc(OCCCn2ccnn2)cc1] | 1735 |
| 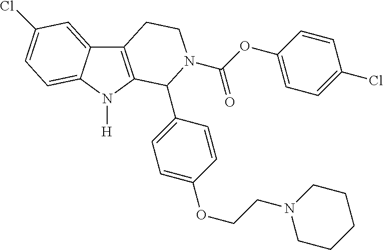 [SMILES: O=C(Oc1ccc(Cl)cc1)N1CCc2c([nH]c3ccc(Cl)cc23)C1c1ccc(OCCN2CCCCC2)cc1] | 1736 |
| 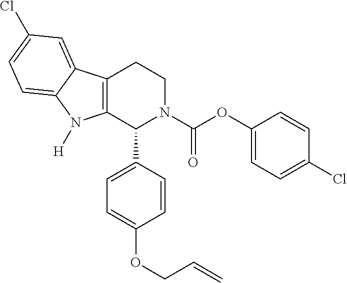 [SMILES: C=CCOc1ccc([C@@H]2c3[nH]c4ccc(Cl)cc4c3CCN2C(=O)Oc2ccc(Cl)cc2)cc1] | 1737 |
| 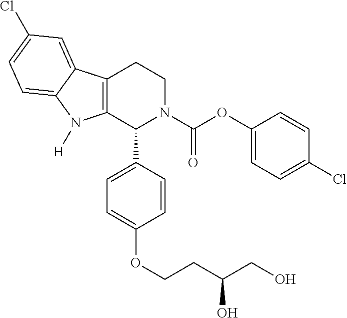 [SMILES: O=C(Oc1ccc(Cl)cc1)N1CCc2c([nH]c3ccc(Cl)cc23)[C@H]1c1ccc(OCC[C@H](O)CO)cc1] | 1738 |

or a pharmaceutically acceptable salt, racemate or stereoisomer thereof.

6.2 Methods of Preparation

Compounds can be prepared by those skilled in the art using known methods, including those set forth in International Publication Nos. WO 2005/089764, WO 2006/113703, WO 2008/127715, WO 2008/127714, WO 2010/138644 and WO 2010/138758, each of which is incorporated by reference herein in its entirety.

6.3 Pharmaceutical Properties and Formulations

6.3.1 Activity

Without being bound by any theory, Compounds described herein inhibit viral infections by inhibiting the production of viral RNA or DNA or one or more viral proteins or one or more virus induced cytopathic effects. Several lines of evidence appear to indicate that the precise molecular target of the Compounds is a host cell target rather than a direct viral target. For example, (1) broad spectrum activity against viruses from diverse and not closely related taxa; (2) the failure to select for a resistant HCV replicon despite long-term exposure at inhibitory concentrations of a Compound in cell culture; and (3) the lack of anti-PV activity in an HT-1080 cell line which is resistant to the cell cycle delay induced by a Compound.

6.3.1.1 Prolongation of Early G₁/Early S-Phase Cell Cycle Delay

Provided herein are Compounds that provoke a prolongation of early G₁/early S-Phase cell cycle delay.

A Compound provided herein provokes a late G₁/early S-Phase cell cycle delay, i.e., between the late resting or pre-DNA synthesis phase, and the early DNA synthesis phase. Further characterization indicates that this effect is concentration dependent, occurring at low nanomolar EC₅₀ values. The cell cycle delay and inhibition of viral production of viral RNA or DNA or one or more viral proteins or one or more virus induced cytopathic effects may occur in concert.

6.3.1.2 Inhibition of Viral Replication and the Production of Viral RNA or DNA, Viral Protein or Virus Induced Cytopathic Effects

Provided herein are Compounds that dose-dependently inhibit viral replication or the production of viral RNA or DNA, viral protein or virus induced cytopathic effects in a diverse panel of viruses.

In viral cell lines in which viral RNA or DNA or viral protein production or production of a virus induced cytopathic effect is decreased by a Compound, further characterization indicates that inhibition of viral replication and production of viral RNA or DNA, viral protein or virus induced cytopathic effects is concentration dependent. Without being bound by any particular theory, the Compound appears to inhibit viral replication and production of viral RNA or DNA, viral protein or virus induced cytopathic effects by interfering with the biological processes of the host cell to inhibit or prevent the formation of a viral replication complex in a cell or in the ER. The interference of the Compound with the biological processes of the host cell is supported by data that includes: (1) broad spectrum activity against viruses from diverse and not closely related taxa; (2) the failure to select a resistant viral replicon despite long-term exposure at inhibitory concentrations of a Compound in cell culture; and (3) the lack of antiviral activity in a cell line which is resistant to the cell cycle delay induced by the Compound. Thus, these experiments indicate that the effects of the Compound on the host cell processes occur in parallel with the effects on viral replication and production of viral RNA or DNA, viral protein or virus induced cytopathic effects.

6.4 Formulations

Compounds can be formulated by those skilled in the art using known methods, including those set forth in International Publication Nos. WO 2005/089764, WO 2006/113703, WO 2008/127715, WO 2008/127714, and WO 2010/138758, each of which is incorporated by reference herein in its entirety.

The Compounds provided herein can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient selected from fillers or diluents, binders, disintegrants, lubricants, flavoring agents, preservatives, stabilizers, suspending agents, dispersing agents, surfactants, antioxidants or solubilizers.

Excipients that may be selected are known to those skilled in the art and include, but are not limited to fillers or diluents (e.g., sucrose, starch, mannitol, sorbitol, lactose, glucose, cellulose, talc, calcium phosphate or calcium carbonate and the like), a binder (e.g., cellulose, carboxymethylcellulose, methylcellulose, hydroxymethylcellulose, hydroxypropylmethylcellulose, polypropylpyrrolidone, polyvinylpyrrolidone, gelatin, gum arabic, polyethyleneglycol or starch and the like), a disintegrants (e.g., sodium starch glycolate, croscarmellose sodium and the like), a lubricant (e.g., magnesium stearate, light anhydrous silicic acid, talc or sodium lauryl sulfate and the like), a flavoring agent (e.g., citric acid, or menthol and the like), a preservative (e.g., sodium benzoate, sodium bisulfite, methylparaben or propylparaben and the like), a stabilizer (e.g., citric acid, sodium citrate or acetic acid and the like), a suspending agent (e.g., methylcellulose, polyvinyl pyrrolidone or aluminum stearate and the like), a dispersing agent (e.g., hydroxypropylmethylcellulose and the like), surfactants (e.g., sodium lauryl sulfate, polaxamer, polysorbates and the like), antioxidants (e.g., ethylene diamine tetraacetic acid (EDTA), butylated hydroxyl toluene (BHT) and the like) and solubilizers (e.g., polyethylene glycols, SOLUTOL®, GELUCIRE® and the like). The effective amount of the Compound provided herein in the pharmaceutical composition may be at a level that will exercise the desired effect.

In any given case, the amount of the Compound provided herein administered will depend on such factors as the solubility of the active component, the formulation used and the route of administration.

The Compound provided herein can be formulated for any route of administration. In a specific embodiment the Compound provided herein is formulated for intradermal, intramuscular, intraperitoneal, percutaneous, intravenous, subcutaneous, intranasal, epidural, sublingual, intracerebral, intravaginal, transdermal, rectal, or mucosal administration, for inhalation, or topical administration to the ears, nose, eyes, or skin. The mode of administration is left to the discretion of the health-care practitioner, and can depend in-part upon the site of the medical condition.

In one embodiment, the Compound provided herein is administered orally using a capsule dosage form composition, wherein the capsule contains the Compound provided herein without an additional carrier, excipient or vehicle.

In another embodiment, provided herein are compositions comprising an effective amount of a Compound provided herein and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

Compositions can be formulated to contain a daily dose, or a convenient fraction of a daily dose, in a dosage unit. In general, the composition is prepared according to known methods in pharmaceutical chemistry. Capsules can be prepared by mixing a Compound provided herein with a suitable carrier or diluent and filling the proper amount of the mixture in capsules.

6.5 Methods of Use

Presented herein are methods for inhibiting or reducing production of viral RNA or DNA or one or more viral proteins or one or more virus induced cytopathic effects.

Presented herein are methods for treating a viral infection by inhibiting or reducing viral replication or the production of viral RNA or DNA, viral protein or virus induced cytopathic effect. In a specific embodiment, a method for inhibiting or reducing viral replication or the production of viral RNA or DNA, viral protein or virus induced cytopathic effects comprises contacting a Compound or a composition thereof with a cell or cell line that produces a virus or viral RNA or DNA, viral protein or virus induced cytopathic effect or may be induced to produce the virus or viral RNA or DNA, viral protein or virus induced cytopathic effect. The cell or cell line may be a virus-infected cell that constitutively produces a virus or viral RNA or DNA, viral protein or virus induced cytopathic effect. Alternatively, or in addition, the cell or cell line may be induced to produce a virus or viral RNA or DNA, viral protein or virus induced cytopathic effect by, e.g., exposure to an active virus. Non-limiting examples of viral cell lines include Huh7, HeLa, Vero, Vero E6, MDCK, MT-2, human peripheral blood mononuclear cells (PBMCs) and the like. In another embodiment, a method for treating a viral infection by inhibiting or reducing viral replication or the production of viral RNA or DNA, viral protein or virus induced cytopathic effects in a subject, comprises administering to a subject a Compound or a composition thereof. In certain embodiments, the subject has a viral infection or a condition associated with viral replication or the production of viral RNA or DNA, viral protein or virus induced cytopathic effects. In specific embodiments, the subject is diagnosed with a viral infection associated with viral replication or the production of viral RNA or DNA, viral protein or virus induced cytopathic effects.

In specific embodiments, the methods for treating a viral infection by inhibiting or reducing viral replication or the production of viral RNA or DNA, viral protein or virus induced cytopathic effects provided herein inhibit or decrease viral replication or the production of viral RNA or DNA, viral protein or virus induced cytopathic effects by at least about 5%, 10%, 15%, 20%, 25%, 30%, 35%, 40%, 45%, 50%, 55%, 60%, 65%, 80%, 85%, 90%, 95%, or 100% relative to viral replication or the production of viral RNA or DNA, viral protein or virus induced cytopathic effects prior to administration of a Compound, as assessed by methods well known in the art. In particular embodiments, the methods for inhibiting or reducing viral replication or the production of viral RNA or DNA, viral protein or virus induced cytopathic effects provided herein inhibit or decrease viral replication or the production of viral RNA or DNA, viral protein or virus induced cytopathic effects in the range of about 5% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, 40% to 100%, relative to viral replication or the production of viral RNA or DNA, viral protein or virus induced cytopathic effects prior to administration of a Compound or any range in between, as assessed by methods well known in the art.

Methods for treating viral infections are also presented herein. In one aspect, the methods for treating a viral infection involve the administration of a Compound, as a single agent therapy, to a patient in need thereof. In a specific embodiment, presented herein is a method for treating a viral infection, comprising administering to a patient in need thereof an effective amount of a Compound, as a single agent. In another embodiment, presented herein is a method for treating a viral infection, comprising administering to a patient in need thereof a pharmaceutical composition comprising a Compound, as the single active ingredient, and a pharmaceutically acceptable carrier, excipient or vehicle.

In another aspect, the methods for treating a viral infection involve the administration of a Compound in combination with another therapy (e.g., one or more additional therapies that do not comprise a Compound, or that comprise a different Compound) to a patient in need thereof. Such methods may involve administering a Compound prior to, concurrent with, or subsequent to administration of the additional therapy. In certain embodiments, such methods have an additive or synergistic effect. In a specific embodiment, presented herein is a method for treating a viral infection, comprising administering to a patient in need thereof an effective amount of a Compound and an effective amount of another therapy.

In specific embodiments, any condition that is associated with a viral infection may be associated with viral replication or the production of viral RNA or DNA, viral protein or virus induced cytopathic effects and can be treated in accordance with the methods provided herein.

In another embodiment, viral infections that can be treated in accordance with the methods described herein include those associated with (+) strand RNA or (−) strand RNA viruses belonging to the families Bunyaviridae, Coronaviridae, Filoviridae, Flaviviridae, Paramyxoviridae, Picornaviridae, Orthomyxoviridae or Rhabdoviridae. Other embodiments include viral infections associated with double-stranded RNA viruses belonging to the family Reoviridae or viruses belonging to the families Retroviridae or Hepadnaviridae. Another embodiment includes viral infections by DNA viruses belonging to the families Adenoviridae, Herpesviridae, Papillomaviridae or Papovaviridae.

Certain examples of viral infections that can be treated in accordance with the methods described herein include viral infections, including but not limited to, those associated with viruses belonging to Flaviviridae (such as West Nile virus (WNV), hepatitis C virus (HCV), yellow fever virus (YFV) and dengue virus (DENV)), Paramyxoviridae (such as parainfluenza virus and respiratory syncytial virus (RSV)), Picornaviridae (such as poliovirus (PV), hepatitis A virus (HAV), Coxsackievirus and rhinovirus), Coronaviridae (such as severe acute respiratory syndrome coronavirus (SARS-CoV)), Orthomyxoviridae (such as influenza virus), or Filoviridae (such as Ebola and Marburg viruses). In one embodiment, the term refers to viral infections by members of the family Retroviridae (such as human immunodeficiency virus (HIV) and human T cell leukemia viruses (HTLV)), Hepadnaviridae (such as hepatitis B virus (HBV)). In another embodiment, the term refers to viral infections by DNA viruses (such as herpes simplex virus (HSV), Kaposi's sarcoma-associated herpesvirus, adenovirus, vaccinia virus or human papilloma virus (HPV)).

In one embodiment, the viral infection is by WNV, HCV, YFV, DENV, RSV, PV, SARS-CoV, influenza virus, parainfluenza virus, HIV, human T cell leukemia viruses, HSV or vaccinia virus. In another embodiment, the viral infection is by WNV, HCV, YFV, DENV, RSV, PV, influenza virus, parainfluenza virus or HIV. In another embodiment, the viral infection is by a known or unknown genotype of HCV. In another embodiment, the genotype of HCV is selected from HCV genotype 1a, HCV genotype 1b or HCV genotype 2a.

The concentration of viral RNA or DNA, viral protein or degree of virus induced cytopathic effects in a biological specimen (e.g., plasma, serum, urine, or any other biofluids or tissues) may be used to monitor the efficacy of a course of treatment for viral infection involving the administration of a compound that inhibits or reduces viral replication or the production of viral RNA or DNA, viral protein or virus induced cytopathic effects, such as a Compound described herein or a compound described in U.S. Publication No. 2005-0272759 (having corresponding International Application Publication No. WO2005/089764), U.S. Publication No. 2005-0282849 (having corresponding International Application Publication No. WO2006/113703), U.S. Publication No. 2007-0254878 (having corresponding International Application Publication No. WO2008/127715) or International Application Publication No. WO2008/127714, each of which is incorporated herein by reference in its entirety. The dosage, frequency and/or length of administration of a Compound or a pharmaceutical composition thereof to a patient may also be modified as a result of the concentration of viral RNA or DNA or viral protein or the production or activity of virus induced cytopathic effects. Alternatively, the changes in the concentration of viral RNA or DNA or viral protein or the production or activity of virus induced cytopathic effects might indicate that the course of treatment involving the administration of the Compound or pharmaceutical composition thereof is effective in treating the viral infection.

In certain embodiments, the concentration of viral RNA or DNA or viral protein or the production or activity of virus induced cytopathic effects in biological specimens (e.g., plasma, serum, urine, or any other biofluids or tissues) of a patient is monitored before, during and/or after a course of treatment for viral infection involving the administration of a Compound or a pharmaceutical composition thereof to the patient. In certain embodiments, the viral titer in a patient is monitored before, during and/or after a course of treatment for viral infection involving the administration of a Compound or a pharmaceutical composition thereof. The dosage, frequency and/or length of administration of a Compound or a pharmaceutical composition thereof to a patient might be modified as a result of the concentration of viral RNA or DNA or viral protein or the production or activity of virus induced cytopathic effects as assessed by standard techniques. Alternatively, the changes in the concentration of viral RNA or DNA or viral protein or the production or activity of virus induced cytopathic effects might indicate that the course of treatment involving the administration of the Compound or pharmaceutical composition thereof is effective in treating the viral infection.

In a specific embodiment, presented herein is a method for treating a viral infection, comprising: (a) administering to a patient in need thereof one or more doses of a Compound or a pharmaceutical composition thereof; and (b) monitoring the concentration of viral RNA or DNA or viral protein or the production or activity of virus induced cytopathic effects (e.g., detected in biological specimens such as plasma, serum, urine, or any other biofluids or tissues) before and/or after step (a). In specific embodiments, step (b) comprises monitoring the patient's viral titer. In certain embodiments, the monitoring step (b) is carried out before and/or after a certain number of doses (e.g., 1, 2, 4, 6, 8, 10, 12, 14, 15, 30 or more doses, or more doses; 2 to 4, 2 to 8, 2 to 20 or 2 to 30 or more doses) or a certain time period (e.g., 1, 2, 3, 4, 5, 6, or 7 days; or 1, 2, 3, 4, 5, 10, 15, 20, 30, 40, 45, 48, or 50 weeks) of administering the Compound. In certain embodiments, one or more of these monitoring parameters are detected prior to administration of the Compound or pharmaceutical composition thereof. In specific embodiments, a decrease in the concentration of viral RNA or DNA or viral protein or the production or activity of virus induced cytopathic effects following administration of the Compound or pharmaceutical composition thereof indicates that the course of treatment is effective for treating the viral infection. In some embodiments, a change in the concentration of viral RNA or DNA or viral protein or the production or activity of virus induced cytopathic effects following administration of the Compound or pharmaceutical composition thereof may indicate that the dosage, frequency and/or length of administration of the Compound or a pharmaceutical composition thereof may be adjusted (e.g., increased, reduced or maintained).

The concentration of viral RNA or DNA or viral protein or the production or activity of virus induced cytopathic effects in a patient may be detected by any technique known to one of skill in the art. In certain embodiments, the method for detecting the concentration of viral RNA or DNA or viral protein or the production or activity of virus induced cytopathic effects in a patient involves obtaining a biological sample (e.g., tissue or fluid sample) from the patient and detecting the concentration of viral RNA or DNA or viral protein or the production or activity of virus induced cytopathic effects in the biological sample (e.g., from plasma, serum, urine, or any other biofluids or tissues), that has been subjected to certain types of treatment (e.g., centrifugation), and detection by use of standard molecular techniques known to a person of ordinary skill in the art, such as by polymerase chain reaction (PCR) or ELISA. In a specific embodiment, an ELISA may be used to detect the concentration of viral protein. In another specific embodiment, PCR may be used to detect the concentration of viral RNA or DNA or viral protein or the production or activity of virus induced cytopathic effects in a biological sample (e.g., from plasma, serum, urine, or any other biofluids or tissues) that has been subjected to certain types of treatment (e.g., centrifugation). Other techniques known in the art that may be used to detect the concentration of viral RNA or DNA or viral protein or the production or activity of virus induced cytopathic effects in a biological sample, including nucleic acid hybridization or a combination of PCR and nucleic acid hybridization assays.

In specific embodiments, the methods for treating a viral infection provided herein alleviate or manage one, two or more symptoms associated with the viral infection. Alleviating or managing one, two or more symptoms of viral infection may be used as a clinical endpoint for efficacy of a Compound for treating the viral infection. In some embodiments, the methods for treating a viral infection provided herein reduce the duration and/or severity of one or more symptoms associated with the viral infection. In some embodiments, the methods for treating viral infection provided herein inhibit the onset, progression and/or recurrence of one or more symptoms associated with the viral infection. In some embodiments, the methods for treating the viral infection provided herein reduce the number of symptoms associated with the viral infection.

The methods for treating a viral infection provided herein inhibit or reduce viral replication or the production of viral RNA or DNA or viral protein or DNA, viral protein or virus induced cytopathic effects. In specific embodiments, the methods for treating the viral infection provided herein selectively inhibit the production of viral RNA or DNA, viral protein or virus induced cytopathic effects. In a specific embodiment, the treatment does not result in an adverse event as defined in according to government safety standards or regulations.

In specific embodiments, the methods for treating a viral infection provided herein inhibit or decrease viral replication or the production of viral RNA or DNA, viral protein or virus induced cytopathic effects by at least about 5%, 10%, 15%, 20%, 25%, 30%, 35%, 40%, 45%, 50%, 55%, 60%, 65%, 80%, 85%, 90%, 95%, or 100% relative to viral replication or the production of viral RNA or DNA, viral protein or virus induced cytopathic effects observed prior to the administration of a Compound as assessed by methods well known in the art, e.g., PCR or ELISA. In particular embodiments, the methods for treating the viral infection provided herein inhibit or decrease viral replication or the production of viral RNA or DNA, viral protein or virus induced cytopathic effects in the range of about 5% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, 30% to 100%, or any range in between, relative to viral replication or the production of viral RNA or DNA, viral protein or virus induced cytopathic effects observed prior to administration of a compound, as assessed by methods well known in the art, e.g., PCR or ELISA.

In some embodiments, the methods for treating a viral infection provided herein reduce, ameliorate, or alleviate the severity of the viral infection and/or one or more symptoms thereof. In other embodiments, the methods for treating viral infection provided herein reduce hospitalization (e.g., the frequency or duration of hospitalization) of a subject diagnosed with the viral infection. In some embodiments, the methods for treating a viral infection provided herein reduce hospitalization length of a subject diagnosed with the viral infection. In certain embodiments, the methods provided herein increase the survival of a subject diagnosed with the viral infection. In specific embodiments, the methods provided herein increase the survival of a subject diagnosed with a viral infection by about 6 months or more, about 7 months or more, about 8 months or more, about 9 months or more, or about 12 months or more. In particular embodiments, the methods for treating a viral infection provided herein inhibit or reduce the progression of the viral infection, or one or more symptoms associated therewith. In specific embodiments, the methods for treating viral infection provided herein enhance or improve the therapeutic effect of another therapy (e.g., an antiviral agent, drug therapy, such as interferon, or transplant surgery). In certain embodiments, the methods for treating viral infection provided herein involve the use of a Compound as an adjuvant therapy. In some embodiments, the methods for treating viral infection provided herein prevent recurrence of the viral infection or one or more symptoms associated with the viral infection.

In particular embodiments, the methods for treating viral infection provided herein reduce the mortality of subjects diagnosed with the viral infection. In certain embodiments, the methods for treating a viral infection provided herein increase the number of patients in remission or decrease the hospitalization rate. In other embodiments, the methods for treating viral infection provided herein prevent the development, onset or progression of one or more symptoms associated with the viral infection. In particular embodiments, the methods for treating the viral infection provided herein increase symptom-free survival of the infected patients. In some embodiments, the methods for treating the viral infection provided herein do not cure the viral infection in patients, but prevent the progression or worsening of the disease. In some embodiments, the methods for treating viral infection provided herein improve the patient's quality of life.

In particular embodiments, the methods for treating viral infection provided herein inhibit, reduce, diminish, arrest, or stabilize the production of viral RNA or DNA or viral protein or the production or activity of a virus induced cytopathic effect associated with the virus. In certain embodiments, the methods for treating viral infection provided herein reduce viral RNA or DNA or viral protein production or the production or activity of a virus induced cytopathic effect in a subject by at least about 5%, 10%, 15%, 20%, 25%, 30%, 35%, 40%, 45%, 50%, 55%, 60%, 65%, 80%, 85%, 90%, 95%, 99%, or 100%, relative to viral RNA or DNA or viral protein production or the production or activity of a virus induced cytopathic effect prior to administration of a Compound as assessed by methods well known in the art, e.g., PCR or ELISA. In particular embodiments, the methods for treating viral infection provided herein reduce the viral titer in a subject by an amount in the range of about 5% to 20%, 10% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, 30% to 100%, or any range in between, relative to the viral titer in a subject prior to administration of a Compound as assessed by methods well known in the art, e.g., PCR or ELISA.

In specific embodiments, the methods for treating viral infection provided herein decrease the number of circulating viral proteins (CVPs) in the blood of the subject as assessed by methods known in the art. In specific embodiments, the methods for treating viral infection provided herein decrease the number of CVPs in the blood of a subject by at least about 5%, 10%, 15%, 20%, 25%, 30%, 35%, 40%, 45%, 50%, 55%, 60%, 65%, 80%, 85%, 90%, 95%, 99%, or 100%, relative to the number of CVPs observed prior to administration of a Compound, as assessed by methods well known in the art.

In certain embodiments, the methods for treating viral infection provided herein increase the viral-free survival rate of patients diagnosed with the viral infection. In some embodiments, the methods for treating viral infection provided herein increase relapse-free survival. In certain embodiments, the methods for treating viral infection provided herein increase the number of patients in remission. In other embodiments, the methods for treating viral infection provided herein increase the length of remission in patients.

In specific embodiments, the methods for treating viral infection provided herein minimize the severity and/or frequency of one or more side effects observed with current antiviral therapies. In certain embodiments, the methods for treating viral infection provided herein do not cause one or more side effects observed with current antiviral therapies.

6.6 Patient Population

In some embodiments, a subject treated for a viral infection in accordance with the methods provided herein is a human who has or is diagnosed with a viral infection. In other embodiments, a subject treated for a viral infection in accordance with the methods provided herein is a human predisposed or susceptible to a viral infection. In some embodiments, a subject treated for a viral infection in accordance with the methods provided herein is a human at risk of developing a viral infection.

In one embodiment, a subject treated for a viral infection in accordance with the methods provided herein is a human infant. In another embodiment, a subject treated for a viral infection in accordance with the methods provided herein is a human toddler. In another embodiment, a subject treated for a viral infection in accordance with the methods provided herein is a human child. In another embodiment, a subject treated for a viral infection in accordance with the methods provided herein is a human adult. In another embodiment, a subject treated for a viral infection in accordance with the methods provided herein is a middle-aged human. In another embodiment, a subject treated for a viral infection in accordance with the methods provided herein is an elderly human.

In certain embodiments, a subject treated for a viral infection in accordance with the methods provided herein is a human that is about 1 to about 5 years old, about 5 to 10 years old, about 10 to about 18 years old, about 18 to about 30 years old, about 25 to about 35 years old, about 35 to about 45 years old, about 40 to about 55 years old, about 50 to about 65 years old, about 60 to about 75 years old, about 70 to about 85 years old, about 80 to about 90 years old, about 90 to about 95 years old or about 95 to about 100 years old, or any age in between. In a specific embodiment, a subject treated for a viral infection in accordance with the methods provided herein is a human that is 18 years old or older. In a particular embodiment, a subject treated for a viral infection in accordance with the methods provided herein is a human child that is between the age of 1 year old to 18 years old. In a certain embodiment, a subject treated for a viral infection in accordance with the methods provided herein is a human that is between the age of 12 years old and 18 years old. In a certain embodiment, the subject is a male human. In another embodiment, the subject is a female human. In one embodiment, the subject is a female human that is not pregnant or is not breastfeeding. In one embodiment, the subject is a female that is pregnant or will/might become pregnant, or is breast feeding.

In particular embodiments, a subject treated for a viral infection in accordance with the methods provided herein is a human that is in an immunocompromised state or immunosuppressed state. In certain embodiments, a subject treated for a viral infection in accordance with the methods provided herein is a human receiving or recovering from immunosuppressive therapy. In certain embodiments, a subject treated for a viral infection in accordance with the methods provided herein is a human that has or is at risk of getting a viral infection, AIDS, or a bacterial infection. In certain embodiments, a subject treated for a viral infection in accordance with the methods provided herein is a human who is, will or has undergone surgery, drug therapy, such as chemotherapy, hormonal therapy and/or radiation therapy.

In some embodiments, a subject treated for a viral infection in accordance with the methods provided herein is administered a Compound or a pharmaceutical composition thereof, or a combination therapy before any adverse effects or intolerance to therapies other than the Compound develops. In some embodiments, a subject treated for a viral infection in accordance with the methods provided herein is a refractory patient. In a certain embodiment, a refractory patient is a patient refractory to a standard therapy (e.g., surgery, radiation, anti-androgen therapy and/or drug therapy such as chemotherapy or antiviral therapy). In certain embodiments, a patient with a viral infection is refractory to a therapy when the viral infection has not significantly been eradicated and/or the one or more symptoms have not been significantly alleviated. The determination of whether a patient is refractory can be made either in vivo or in vitro by any method known in the art for assaying the effectiveness of a treatment of a viral infection, using art-accepted meanings of “refractory” in such a context.

In some embodiments, a subject treated for a viral infection in accordance with the methods provided herein is a human that has proven refractory to therapies other than treatment with a Compound, but is no longer on these therapies. In certain embodiments, a subject treated for a viral infection in accordance with the methods provided herein is a human already receiving one or more conventional therapies, such as surgery, drug therapy or antiviral therapy. Among these patients are refractory patients, patients who are too young for conventional therapies, and patients with recurring tumors or viral infection despite treatment with existing therapies.

In some embodiments, a subject treated for a viral infection in accordance with the methods provided herein is a human susceptible to adverse reactions to conventional therapies. In some embodiments, a subject treated for a viral infection in accordance with the methods provided herein is a human that has not received a therapy, e.g., drug therapy such as chemotherapy, surgery, antiviral therapy, anti-androgen therapy or radiation therapy, prior to the administration of a Compound or a pharmaceutical composition thereof. In other embodiments, a subject treated for a viral infection in accordance with the methods provided herein is a human that has received a therapy prior to administration of a Compound. In some embodiments, a subject treated for a viral infection in accordance with the methods provided herein is a human that has experienced adverse side effects to the prior therapy or the prior therapy was discontinued due to unacceptable levels of toxicity to the human.

In some embodiments, a subject treated for a viral infection in accordance with the methods provided herein is not, has not and/or will not receive a drug that is primarily metabolized by CYP2D6. In particular embodiments, a subject treated for a viral infection in accordance with the methods provided herein has not and will not received a drug that is primarily metabolized by CYP2D6 1, 2, 3 or 4 weeks before receiving a Compound or a pharmaceutical composition thereof and 1, 2, 3 or 4 weeks after receiving the Compound or pharmaceutical composition. Examples of such drugs include, without limitation, some antidepressants (e.g., tricyclic antidepressants and selective serotonin uptake inhibitors), some antipsychotics, some beta-adrenergic receptor blockers, certain antiviral agents and certain anti-arrhythmics.

6.7 Dosage and Administration

In accordance with the methods for treating a viral infection provided herein, a Compound or a pharmaceutical composition thereof can be administered to a subject in need thereof by a variety of routes in amounts which result in a beneficial or therapeutic effect. A Compound or pharmaceutical composition thereof may be orally administered to a subject in need thereof in accordance with the methods for treating a viral infection provided herein. The oral administration of a Compound or a pharmaceutical composition thereof may facilitate subjects in need of such treatment complying with a regimen for taking the Compound or pharmaceutical composition. Thus, in a specific embodiment, a Compound or pharmaceutical composition thereof is administered orally to a subject in need thereof.

A Compound provided herein can be administered orally, with or without food or water.

Other routes of administration include, but are not limited to, intravenous, intradermal, intrathecal, intramuscular, subcutaneous, intranasal, inhalation, transdermal, topical, transmucosal, intracranial, intratumoral, epidural and intra-synovial. In one embodiment, a Compound or a pharmaceutical composition thereof is administered systemically (e.g., parenterally) to a subject in need thereof. In another embodiment, a Compound or a pharmaceutical composition thereof is administered locally (e.g., intratumorally) to a subject in need thereof. In one embodiment, a Compound or a pharmaceutical composition thereof is administered via a route that permits the Compound to cross the blood-brain barrier (e.g., orally).

In accordance with the methods for treating a viral infection provided herein that involve administration of a Compound in combination with one or more additional therapies, the Compound and one or more additional therapies may be administered by the same route or a different route of administration.

The dosage and frequency of administration of a Compound or a pharmaceutical composition thereof is administered to a subject in need thereof in accordance with the methods for treating a viral infection provided herein will be efficacious while minimizing any side effects. The exact dosage and frequency of administration of a Compound or a pharmaceutical composition thereof can be determined by a practitioner, in light of factors related to the subject that requires treatment. Factors which may be taken into account include the severity of the disease state, general health of the subject, age, weight, and gender of the subject, diet, time and frequency of administration, drug combination(s), reaction sensitivities, and tolerance/response to therapy. The dosage and frequency of administration of a Compound or a pharmaceutical composition thereof may be adjusted over time to provide sufficient levels of the Compound or to maintain the desired effect.

In certain embodiments, a Compound or pharmaceutical composition thereof is administered to a subject in accordance with the methods for treating a viral infection presented herein once a day, twice a day, three times a day, or four times a day. In some embodiments, a Compound or pharmaceutical composition thereof is administered to a subject in accordance with the methods for treating a viral infection presented herein once, twice, three times, or four times every other day (i.e., on alternate days), once, twice, three times, or four times every two days, once every three days, once, twice, three times, or four times every four days, once, twice, three times, or four times every 5 days, once, twice, three times, or four times a week, once, twice, three times, or four times every two weeks, once, twice, three times, or four times every three weeks, once, twice, three times, or four times every four weeks, once, twice, three times, or four times every 5 weeks, once, twice, three times, or four times every 6 weeks, once, twice, three times, or four times every 7 weeks, or once, twice, three times, or four times every 8 weeks. In particular embodiments, a Compound or pharmaceutical composition thereof is administered to a subject in accordance with the methods for treating a viral infection presented herein in cycles, wherein the Compound or pharmaceutical composition is administered for a period of time, followed by a period of rest (i.e., the Compound or pharmaceutical composition is not administered for a period of time).

In certain embodiments, a Compound or a pharmaceutical composition thereof is administered to a subject in need thereof in accordance with the methods for treating a viral infection provided herein at a dosage and a frequency of administration that achieves one or more of the following: (i) decreases the production or concentration of viral RNA or DNA or viral protein or the production or activity of a virus induced cytopathic effect; (ii) decreases the viral titer of a subject or an animal model with a viral infection; (iii) reduces or ameliorates the severity of the viral infection and/or one or more symptoms associated therewith in a subject with the viral infection; (iv) reduces the number symptoms and/or the duration of one or more symptoms associated with the viral infection in a subject with the viral infection; (v) prevents the onset, progression or recurrence of one or more symptoms associated with the viral infection in a subject with the viral infection; (vi) inhibits or reduces viral replication or the production or concentration of viral RNA or DNA or viral protein or the production or activity of a virus induced cytopathic effect associated with the viral infection in a subject or an animal model; and/or (vii) enhances or improves the therapeutic efficacy of another antiviral therapy in a subject with the viral infection or an animal model.

In one aspect, a method for treating a viral infection presented herein involves the administration of a unit dosage of a Compound or a pharmaceutical composition thereof. The dosage may be administered as often as determined effective (e.g., once, twice or three times per day, every other day, once or twice per week, biweekly or monthly). In certain embodiments, a method for treating a viral infection presented herein involves the administration to a subject in need thereof of a unit dose of a Compound or a pharmaceutical composition thereof that ranges, without limitation, from about 0.001 milligram (mg) per kg to about 1500 mg per kg, from about 0.001 mg per kg to about 1400 mg per kg, from about 0.001 mg per kg to about 1300 mg per kg, from about 0.001 mg per kg to about 1200 mg per kg, from about 0.001 mg per kg to about 1100 mg per kg, from about 0.001 mg per kg to about 1000 mg per kg, from about 0.01 mg to about 1500 mg, from about 0.01 mg per kg to about 1000 mg per kg, from about 0.1 mg per kg to about 1500 mg per kg, from about 0.1 mg per kg to about 1000 mg per kg, from about 0.1 mg per kg to about 500 mg per kg, from about 0.05 mg to about 1000 mg, from about 0.1 mg per kg to about 100 mg per kg, from about 1 mg per kg to about 100 mg per kg, from about 10 mg to about 500 mg, from about 100 mg to about 500 mg, from about 150 mg to about 500 mg, from about 150 mg to about 1000 mg, from about 250 mg to about 1000 mg, from about 300 mg to about 1000 mg, or from about 500 mg to about 1000 mg, or any range in between. In specific embodiments, oral doses for use in the methods provided herein are in a range of, without limitation, from about 0.01 mg to about 300 mg per kg body weight, from about 0.1 mg to about 75 mg per kg body weight, or from about 0.5 mg to 5 mg per kg body weight. In some embodiments, a method for treating a viral infection presented herein involves the administration to a subject in need thereof of a unit dose of a Compound or a pharmaceutical composition thereof, without limitation, of about 15 mg, 16, mg, 17 mg, 18 mg, 19 mg, 20 mg, 21, mg, 22 mg, 23 mg, 24 mg, 25 mg, 26 mg, 27 mg, 28 mg, 29 mg, 30 mg or 40 mg. In certain embodiments, a method for treating a viral infection presented herein involves the administration to a subject in need thereof of a unit dose of a Compound or a pharmaceutical composition thereof, without limitation, of about 50 mg, 60 mg, 70 mg, 80 mg, 90 mg, 100 mg, 110 mg, 120 mg, 125 mg, 130 mg, 140 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1050 mg, 1100 mg, 1150 mg, 1200 mg, 1250 mg, 1300 mg, 1350 mg, 1400 mg, 1450 mg or 1500 mg.

In some embodiments, a method for treating a viral infection presented herein involves the administration to a subject in need thereof of a unit dose of a Compound or a pharmaceutical composition thereof, without limitation, of at least about 0.1 mg, 1 mg, 5 mg, 10 mg, 20 mg, 30 mg, 40 mg, 50 mg, 60 mg, 70 mg, 80 mg, 90 mg, 100 mg, 110 mg, 120 mg, 125 mg, 130 mg, 140 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1050 mg, 1100 mg, 1150 mg, 1200 mg, 1250 mg, 1300 mg, 1350 mg, 1400 mg, 1450 mg, 1500 mg or more. In certain embodiments, a method for treating a viral infection presented herein involves the administration to a subject in need thereof of a unit dose of a Compound or a pharmaceutical composition thereof, without limitation, of less than about 35 mg, less than about 40 mg, less than about 45 mg, less than about 50 mg, less than about 60 mg, less than about 70 mg, or less than about 80 mg.

In specific embodiments, a method for treating a viral infection presented herein involves the administration to a subject in need thereof of a unit dose of a Compound or a pharmaceutical composition thereof in a range, without limitation, of from about 20 mg to about 500 mg, from about 40 mg to about 500 mg, from about 40 mg to about 200 mg, from about 40 mg to about 150 mg, from about 75 mg to about 500 mg, from about 75 mg to about 450 mg, from about 75 mg to about 400 mg, from about 75 mg to about 350 mg, from about 75 mg to about 300 mg, from about 75 mg to about 250 mg, from about 75 mg to about 200 mg, from about 100 mg to about 200 mg, or any range in between. In other specific embodiments, a method for treating a viral infection presented herein involves the administration to a subject in need thereof of a unit dose of a Compound or a pharmaceutical composition thereof, without limitation, of about 20 mg, 35 mg, 40 mg, 50 mg, 60 mg, 75 mg, 100 mg, 125 mg, 150 mg, 175 mg, 200 mg, 225 mg, 250 mg or 300 mg. In some embodiments, a method for treating a viral infection presented herein involves the administration to a subject in need thereof of a unit dose of a Compound or a pharmaceutical composition thereof, without limitation, of about 350 mg, 400 mg, 500 mg, 600 mg, 700 mg, 800 mg, 900 mg, or 1000 mg. In some embodiments, a unit dose of a Compound or a pharmaceutical composition thereof is administered to a subject once per day, twice per day, three times per day; once, twice or three times every other day (i.e., on alternate days); once, twice or three times every two days; once, twice or three times every three days; once, twice or three times every four days; once, twice or three times every five days; once, twice, or three times once a week, biweekly or monthly, and the dosage may be administered orally.

In certain embodiments, a method for treating a viral infection presented herein involves the oral administration to a subject in need thereof of a unit dose of a Compound or a pharmaceutical composition thereof ranges, without limitation, from about 0.001 mg per kg to about 1500 mg per kg per day, from about 0.001 mg per kg to about 1400 mg per kg per day, from about 0.001 mg per kg to about 1300 mg per kg per day, from about 0.001 mg per kg to about 1200 mg per kg per day, from about 0.001 mg per kg to about 1100 mg per kg per day, from about 0.001 mg per kg to about 1000 mg per kg per day, 0.001 mg/kg to about 500 mg/kg, from about 0.01 mg per kg to about 1500 mg per kg per day, from about 0.01 mg per kg to about 1000 mg per kg per day, from about 0.1 mg per kg to about 1500 mg per kg per day, from about 0.1 mg per kg to about 1000 mg per kg per day, from about 0.1 mg per kg to about 500 mg per kg per day, from about 0.1 mg per kg to about 100 mg per kg per day, or from about 1 mg per kg to about 100 mg per kg per day. In a specific embodiment, a unit dose of a Compound or a pharmaceutical composition thereof ranges, without limitation, from about 0.01 mg to about 300 mg per kg body weight per day, from about 0.1 mg to about 75 mg per kg body weight per day, or from about 0.5 mg to 5 mg per kg body weight per day. In another specific embodiment, a unit dose of a Compound or a pharmaceutical composition thereof ranges from about 20 mg to about 1000 mg per day. In some embodiments, a method for treating a viral infection presented herein involves the oral administration to a subject in need thereof of a unit dose of a Compound or a pharmaceutical composition thereof that ranges, without limitation, from about 80 mg to about 800 mg per day, from about 100 mg to about 800 mg per day, from about 80 mg to about 600 mg per day, from about 80 mg to about 400 mg per day, from about 80 mg to about 200 mg per day, from about 200 mg to about 300 mg per day, from about 200 mg to about 400 mg per day, from about 200 mg to about 800 mg per day, or any range in between.

In certain embodiments, a unit dose of a Compound that may be used in the methods provided herein include, without limitation, doses of about 0.1 mg/kg/day, 0.2 mg/kg/day, 0.3 mg/kg/day, 0.4 mg/kg/day, 0.5 mg/kg/day, 0.6 mg/kg/day, 0.7 mg/kg/day, 0.8 mg/kg/day, 0.9 mg/kg/day, 1 mg/kg/day, 1.5 mg/kg/day, 2 mg/kg/day, 2.5 mg/kg/day, 2.75 mg/kg/day, 3 mg/kg/day, 4 mg/kg/day, 5 mg/kg/day, 6 mg/kg/day, 6.5 mg/kg/day, 6.75 mg/kg/day, 7 mg/kg/day, 7.5 mg/kg/day, 8 mg/kg/day, 8.5 mg/kg/day, 9 mg/kg/day, 10 mg/kg/day, 11 mg/kg/day, 12 mg/kg/day, 13 mg/kg/day, 14 mg/kg/day or 15 mg/kg/day. In accordance with these embodiments, the dosage may be administered once, twice or three times per day, every other day, or once or twice per week and the dosage may be administered orally.

In a specific embodiment, a method for treating a viral infection presented herein involves the oral administration of a unit dose of about 20 mg of a Compound or a pharmaceutical composition thereof once, twice or three times per day. In another specific embodiment, a method for treating a viral infection presented herein involves the oral administration to a subject in need thereof of a unit dose of about 40 mg of a Compound or a pharmaceutical composition thereof once, twice or three times per day. In another specific embodiment, a method for treating a viral infection presented herein involves the oral administration of a unit dose of about 60 mg of a compound or a pharmaceutical composition thereof once, twice or three times per day. In another specific embodiment, a method for treating a viral infection presented herein involves the oral administration to a subject in need thereof of a unit dose of about 80 mg of a Compound or a pharmaceutical composition thereof once, twice or three times per day. In specific embodiments, a method for treating a viral infection presented herein involves the oral administration to a subject in need thereof of a unit dose in a range, without limitation, of from about 100 mg to about 250 mg, from about 150 mg to about 250 mg, from about 175 mg to about 250 mg, from about 200 mg to about 250 mg, or from about 200 mg to about 225 mg of a Compound or a pharmaceutical composition thereof once, twice or three times per day.

Non-limiting exemplary doses of a Compound or a pharmaceutical composition that may be used in the methods for treating a viral infection provided herein include milligram (mg) amounts per kilogram (kg) of subject or sample weight. In certain embodiments, a method for treating a viral infection presented herein involves the administration to a subject in need thereof of a dosage of a Compound or a pharmaceutical composition thereof that ranges, without limitation, from about 0.001 mg/kg to about 1500 mg/kg per day, from about 0.001 mg/kg to about 1400 mg/kg per day, from about 0.001 mg/kg to about 1300 mg/kg per day, from about 0.001 mg/kg to about 1200 mg/kg per day, from about 0.001 mg/kg to about 1100 mg/kg per day, from about 0.001 mg/kg to about 1000 mg/kg per day, 0.001 mg/kg to about 500 mg/kg, from about 0.01 mg/kg to about 1500 mg/kg per day, from about 0.01 mg/kg to about 1000 mg/kg per day, from about 0.01 mg/kg to about 500 mg/kg, from about 0.1 mg/kg to about 1500 mg/kg per day, from about 0.1 mg/kg to about 1000 mg/kg per day, from about 0.1 mg/kg to about 500 mg/kg, from about 0.1 mg/kg to about 100 mg/kg per day, from about 1 mg/kg to about 500 mg/kg, from about 1 mg/kg to about 100 mg/kg per day, from about 10 mg/kg to about 500 mg/kg, from about 100 mg to about 500 mg/kg, from about 150 mg/kg to about 500 mg/kg, from about 250 mg/kg to about 500 mg/kg, or from about 300 mg/kg to about 500 mg/kg. In some embodiments, a method for treating a viral infection presented herein involves the administration to a subject in need thereof of a dosage of a Compound or a pharmaceutical composition thereof that ranges, without limitation, from about 0.001 mg/kg to about 100 mg/kg, from about 0.001 mg/kg to about 50 mg/kg, from about 0.001 mg/kg to about 25 mg/kg, from about 0.001 mg/kg to about 10 mg/kg, from about 0.001 mg/kg to about 5 mg/kg; from about 0.001 mg/kg to about 1 mg/kg; or from about 0.001 mg/kg to about 0.01 mg/kg. In certain embodiments, a dosage of a Compound or a pharmaceutical composition thereof that may be used in the methods provided herein include, without limitation, doses of about 0.1 mg/kg/day, 0.2 mg/kg/day, 0.3 mg/kg/day, 0.4 mg/kg/day, 0.5 mg/kg/day, 0.6 mg/kg/day, 0.7 mg/kg/day, 0.8 mg/kg/day, 0.9 mg/kg/day, 1 mg/kg/day, 1.5 mg/kg/day, 2 mg/kg/day, 2.5 mg/kg/day, 2.75 mg/kg/day, 3 mg/kg/day, 4 mg/kg/day, 5 mg/kg/day, 6 mg/kg/day, 6.5 mg/kg/day, 6.75 mg/kg/day, 7 mg/kg/day, 7.5 mg/kg/day, 8 mg/kg/day, 8.5 mg/kg/day, 9 mg/kg/day, 10 mg/kg/day, 11 mg/kg/day, 12 mg/kg/day, 13 mg/kg/day, 14 mg/kg/day or 15 mg/kg/day. In accordance with these embodiments, the dosage may be administered once, twice or three times per day, every other day, or once or twice per week and the dosage may be administered orally.

In certain embodiments, a method for treating a viral infection presented herein involves the administration to a subject in need thereof of a dosage of a Compound or a pharmaceutical composition thereof that ranges, without limitation, from about 0.01 mg/kg to about 100 mg/kg, from about 0.01 mg/kg to about 50 mg/kg, from about 0.01 mg/kg to about 25 mg/kg, from about 0.01 mg/kg to about 10 mg/kg, from about 0.01 mg/kg to about 5 mg/kg, from about 0.01 mg to about 1 mg/kg, or from about 0.01 mg/kg to about 0.1 mg/kg. In some embodiments, a method for treating a viral infection presented herein involves the administration to a subject in need thereof of a dosage of a Compound or a pharmaceutical composition thereof that ranges, without limitation, from about 0.1 mg/kg to about 100 mg/kg, from about 0.1 mg/kg to about 50 mg/kg, from about 0.1 mg/kg to about 25 mg/kg, from about 0.1 mg/kg to about 10 mg/kg, from about 0.1 mg/kg to about 5 mg/kg, from about 0.1 mg/kg to about 4 mg/kg; from about 0.1 mg/kg to about 3 mg/kg; from about 0.1 mg/kg to about 2 mg/kg; from about 0.1 mg to about 1.5 mg/kg, from about 0.1 mg to about 1.2 mg/kg, from about 0.1 mg to about 1 mg/kg, or from about 0.5 mg/kg to about 1.5 mg/kg. In accordance with these embodiments, the dosage may be administered once, twice or three times per day, every other day, or once or twice per week and the dosage may be administered orally.

In specific embodiments, a method for treating a viral infection presented herein involves the oral administration to a subject in need thereof of a dosage of a Compound or a pharmaceutical composition thereof in a range, without limitation, of from about 0.1 mg/kg to about 5 mg/kg, from about 0.1 mg/kg to about 4 mg/kg, from about 0.1 mg/kg to about 3 mg/kg, from about 0.1 mg/kg to about 2 mg/kg, from about 0.5 mg/kg to about 2 mg/kg, or from about 1 mg/kg to about 1.5 mg/kg is administered once, twice or three times per day. In certain embodiments, a method for treating a viral infection presented herein involves the oral administration to a subject in need thereof of a dosage of a Compound or a pharmaceutical composition thereof, without limitation, of about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg or about 1 mg/kg once, twice or three times per day. In certain specific embodiments, a method for treating a viral infection presented herein involves the oral administration to a subject in need thereof of a dosage of a Compound or a pharmaceutical composition thereof, without limitation, of about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg or about 2 mg/kg once, twice or three times per day.

In specific aspects, a method for treating a viral infection presented herein involves the administration to a subject in need thereof of a Compound or a pharmaceutical composition thereof at a dosage that achieves a target plasma concentration of the Compound in a subject with a viral infection or an animal model (e.g., an animal model with a pre-established human tumor or a viral infection). In a particular embodiment, a method for treating a viral infection presented herein involves the administration to a subject in need thereof of a Compound or a pharmaceutical composition thereof at a dosage that achieves a plasma concentration of the Compound ranging from, without limitation, approximately 0.001 μg/mL to approximately 100 mg/mL, approximately 0.01 μg/mL to approximately 100 mg/mL, approximately 0.01 μg/mL to approximately 10 mg/mL, approximately 0.1 μg/mL to approximately 10 mg/mL, approximately 0.1 μg/mL to approximately 500 μg/mL, approximately 0.1 μg/mL to approximately 200 μg/mL, approximately 0.1 μg/mL to approximately 100 μg/mL, or approximately 0.1 μg/mL to approximately 75 μg/mL in a subject with the viral infection or an animal model (e.g., an animal model with a pre-established human tumor or viral infection). In specific embodiments, a method for treating a viral infection presented herein involves the administration to a subject in need thereof of a Compound or a pharmaceutical composition thereof at a dosage that achieves a plasma concentration of the Compound ranging from, without limitation, approximately 0.1 to approximately 50 μg/mL, approximately 0.1 μg/mL to approximately 25 μg/mL, approximately 0.1 μg/mL to approximately 20 μg/mL or approximately 5 μg/mL to approximately 10 μg/mL in a subject with the viral infection or an animal model (e.g., an animal model with a pre-established human tumor or viral infection). To achieve such plasma concentrations, a Compound or a pharmaceutical composition thereof may be administered at doses that vary in a range, without limitation, from 0.001 μg to 100,000 mg, depending upon the route of administration. In certain embodiments, subsequent doses of a Compound may be adjusted accordingly based on the plasma concentrations of the Compound achieved with initial doses of the Compound or pharmaceutical composition thereof administered to the subject.

In particular embodiments, a method for treating a viral infection presented herein involves the administration to a subject in need thereof of a Compound or a pharmaceutical composition thereof at a dosage that achieves the desired tissue to plasma concentration ratios of the Compound as determined, e.g., by any imaging techniques known in the art such as whole-body autoradiography, in a subject with the viral infection or an animal model (such as an animal model with a pre-established human tumor or a viral infection).

In some embodiments, a method for treating a viral infection presented herein involves the administration to a subject in need thereof of one or more doses of an effective amount of a Compound or a pharmaceutical composition, wherein the effective amount may or may not be the same for each dose. In particular embodiments, a first dose of a Compound or pharmaceutical composition thereof is administered to a subject in need thereof for a first period of time, and subsequently, a second dose of a Compound is administered to the subject for a second period of time. The first dose may be more than the second dose, or the first dose may be less than the second dose. A third dose of a Compound also may be administered to a subject in need thereof for a third period of time.

In some embodiments, the dosage amounts described herein refer to total amounts administered; that is, if more than one Compound is administered, then, in some embodiments, the dosages correspond to the total amount administered. In a specific embodiment, oral compositions contain about 5% to about 95% of a Compound by weight.

The length of time that a subject in need thereof is administered a Compound or a pharmaceutical composition in accordance with the methods for treating a viral infection presented herein will be the time period that is determined to be efficacious. In certain embodiments, a method for treating a viral infection presented herein involves the administration of a Compound or a pharmaceutical composition thereof for a period of time until the severity and/or number of one or more symptoms associated with the viral infection decrease.

In some embodiments, a method for treating a viral infection presented herein involves the administration of a Compound or a pharmaceutical composition thereof for up to 48 weeks. In other embodiments, a method for treating a viral infection presented herein involves the administration of a Compound or a pharmaceutical composition thereof for up to 4 weeks, 8 weeks, 12 weeks, 16 weeks, 20 weeks, 24 weeks, 26 weeks (0.5 year), 52 weeks (1 year), 78 weeks (1.5 years), 104 weeks (2 years), or 130 weeks (2.5 years) or more. In certain embodiments, a method for treating a viral infection presented herein involves the administration of a Compound or a pharmaceutical composition thereof for an indefinite period of time. In some embodiments, a method for treating a viral infection presented herein involves the administration of a Compound or a pharmaceutical composition thereof for a period of time followed by a period of rest (i.e., a period wherein the Compound is not administered) before the administration of the Compound or pharmaceutical composition thereof is resumed. In specific embodiments, a method for treating a viral infection presented herein involves the administration of a Compound or pharmaceutical composition thereof in cycles, e.g., 1 week cycles, 2 week cycles, 3 week cycles, 4 week cycles, 5 week cycles, 6 week cycles, 8 week cycles, 9 week cycles, 10 week cycles, 11 week cycles, or 12 week cycles. In such cycles, the Compound or a pharmaceutical composition thereof may be administered once, twice, three times, or four times daily. In particular embodiments, a method for treating a prostate condition presented herein involves the administration of a Compound or a pharmaceutical composition thereof twice daily in 4 week cycles.

In certain embodiments, in accordance with the methods for treating a viral infection presented herein, a Compound or a pharmaceutical composition thereof is administered to a subject in need thereof prior to, concurrently with, or after a meal (e.g., breakfast, lunch, or dinner). In specific embodiments, in accordance with the methods for treating a viral infection presented herein, a Compound or a pharmaceutical composition thereof is administered to a subject in need thereof in the morning (e.g., between 5 am and 12 pm). In certain embodiments, in accordance with the methods for treating a viral infection presented herein, a Compound or a pharmaceutical composition thereof is administered to a subject in need thereof at noon (i.e., 12 pm). In particular embodiments, in accordance with the methods for treating a viral infection presented herein, a Compound or a pharmaceutical composition thereof is administered to a subject in need thereof in the afternoon (e.g., between 12 pm and 5 pm), evening (e.g., between 5 pm and bedtime), and/or before bedtime.

In specific embodiments, a dose of a Compound or a pharmaceutical composition thereof is administered to a subject once per day, twice per day, three times per day; once, twice or three times every other day (i.e., on alternate days); once, twice or three times every two days; once, twice or three times every three days; once, twice or three times every four days; once, twice or three times every five days; once, twice, or three times once a week, biweekly or monthly.

6.8 Combination Therapy

Presented herein are combination therapies for the treatment of a viral infection which involve the administration of a Compound in combination with one or more additional therapies to a subject in need thereof. In a specific embodiment, presented herein are combination therapies for the treatment of a viral infection which involve the administration of an effective amount of a Compound in combination with an effective amount of another therapy to a subject in need thereof.

As used herein, the term “in combination,” refers, in the context of the administration of a Compound, to the administration of a Compound prior to, concurrently with, or subsequent to the administration of one or more additional therapies (e.g., agents, surgery, or radiation) for use in treating a viral infection. The use of the term “in combination” does not restrict the order in which one or more Compounds and one or more additional therapies are administered to a subject. In specific embodiments, the interval of time between the administration of a Compound and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, or any period of time in between. In certain embodiments, a Compound and one or more additional therapies are administered less than 1 day, 1 week, 2 weeks, 3 weeks, 4 weeks, one month, 2 months, 3 months, 6 months, 1 year, 2 years, or 5 years apart.

In some embodiments, the combination therapies provided herein involve administering a Compound daily, and administering one or more additional therapies once a week, once every 2 weeks, once every 3 weeks, once every 4 weeks, once every month, once every 2 months (e.g., approximately 8 weeks), once every 3 months (e.g., approximately 12 weeks), or once every 4 months (e.g., approximately 16 weeks). In certain embodiments, a Compound and one or more additional therapies are cyclically administered to a subject. Cycling therapy involves the administration of the Compound for a period of time, followed by the administration of one or more additional therapies for a period of time, and repeating this sequential administration. In certain embodiments, cycling therapy may also include a period of rest where the Compound or the additional therapy is not administered for a period of time (e.g., 2 days, 3 days, 4 days, 5 days, 6 days, 7 days, 1 week, 2 weeks, 3 weeks, 4 weeks, 5 weeks, 10 weeks, 20 weeks, 1 month, 2 months, 3 months, 4 months, 5 months, 6 months, 7 months, 8 months, 9 months, 10 months, 11 months, 12 months, 2 years, or 3 years). In an embodiment, the number of cycles administered is from 1 to 12 cycles, from 2 to 10 cycles, or from 2 to 8 cycles.

In some embodiments, the methods for treating a viral infection provided herein comprise administering a Compound as a single agent for a period of time prior to administering the Compound in combination with an additional therapy. In certain embodiments, the methods for treating a viral infection provided herein comprise administering an additional therapy alone for a period of time prior to administering a Compound in combination with the additional therapy.

In some embodiments, the administration of a Compound and one or more additional therapies in accordance with the methods presented herein have an additive effect relative the administration of the Compound or said one or more additional therapies alone. In some embodiments, the administration of a Compound and one or more additional therapies in accordance with the methods presented herein have a synergistic effect relative to the administration of the Compound or said one or more additional therapies alone.

As used herein, the term “synergistic,” refers to the effect of the administration of a Compound in combination with one or more additional therapies (e.g., agents), which combination is more effective than the additive effects of any two or more single therapies (e.g., agents). In a specific embodiment, a synergistic effect of a combination therapy permits the use of lower dosages (e.g., sub-optimal doses) of a Compound or an additional therapy and/or less frequent administration of a Compound or an additional therapy to a subject. In certain embodiments, the ability to utilize lower dosages of a Compound or of an additional therapy and/or to administer a Compound or said additional therapy less frequently reduces the toxicity associated with the administration of a Compound or of said additional therapy, respectively, to a subject without reducing the efficacy of a Compound or of said additional therapy, respectively, in the treatment of a viral infection. In some embodiments, a synergistic effect results in improved efficacy of a Compound and each of said additional therapies in treating a viral infection. In some embodiments, a synergistic effect of a combination of a Compound and one or more additional therapies avoids or reduces adverse or unwanted side effects associated with the use of any single therapy.

The combination of a Compound and one or more additional therapies can be administered to a subject in the same pharmaceutical composition. Alternatively, a Compound and one or more additional therapies can be administered concurrently to a subject in separate pharmaceutical compositions. A Compound and one or more additional therapies can be administered sequentially to a subject in separate pharmaceutical compositions. A Compound and one or more additional therapies may also be administered to a subject by the same or different routes of administration.

The combination therapies provided herein involve administering to a subject to in need thereof a Compound in combination with conventional, or known, therapies for treating a viral infection. Other therapies for a viral infection or a condition associated therewith are aimed at controlling or relieving one or more symptoms. Accordingly, in some embodiments, the combination therapies provided herein involve administering to a subject to in need thereof a pain reliever, or other therapies aimed at alleviating or controlling one or more symptoms associated with a viral infection or a condition associated therewith.

Non-limiting examples of other therapies that may be used in combination with a Compound for treating a viral infection include a HCV protease inhibitor such as a NS2 protease inhibitor, a NS3 protease inhibitor, a peptide or dipeptide NS3 protease inhibitor or a NS4a protease cofactor inhibitor; a nucleoside or non-nucleoside HCV NS5b polymerase inhibitor; one or more agents such as a NS4b inhibitor, NS5a inhibitor, IRES inhibitor (such as a steroid, a ribozyme, miRNA, siRNA or an antisense RNA), p7 inhibitor, entry inhibitor, fusion inhibitor, helicase inhibitor, ribavirin, a ribavirin analogue, ribavirin and at least one or more of a nonpegylated interferon or a pegylated interferon, a TLR agonist, cyclophilin inhibitor, caspase or pancaspase inhibitor, immunomodulator, immunomodulator/antiinflammatory, antiinflammatory, antiinflammatory/antifibrotic, broad spectrum immune stimulator, antifibrotic, antioxidant, hemopurifier, IMPDH inhibitor, glycosidase inhibitor, glucosidase inhibitor, HCV therapeutic vaccine, A3 adenosine receptor (AR) agonist, polypeptide eglin c analog inhibitor, human pancreatic secretory trypsin and minibody repertoire inhibitor or a monoclonal antibody and fragment thereof; or, one or more different agents such as a HIV inhibitor, HBV inhibitor, RNA inhibitor, RNAi, anti-phospholipid therapy, protein therapeutic, interferon replacement agent, botanical or non-specific pharmaceutical.

A specific non-limiting example of other therapies that may be used in combination with a Compound for treating a viral infection include the NS3 HCV protease inhibitor BI 201335 (Boehringer Ingelheim Pharma), boceprevir (also referred to as SCH-503034, Merck/Schering-Plough Corporation), ciluprevir (also referred to as BILN-2061, Boehringer Ingelheim Pharma), IDX136 (Idenix Pharmaceuticals, Inc.), GS-9256 (Gilead), GS-9451 (Gilead), IDX316 (Idenix Pharmaceuticals, Inc.), ITMN-191 (also referred to as R-7227, InterMune/Roche Pharmaceuticals), MK-7009 (Merck), PHX1766 (Phenomix), SCH-6 (Merck/Schering-Plough Corporation), SCH-900518 (also referred to as SCH-518, Merck/Schering-Plough Corporation), telaprevir (also referred to as VX 950, Vertex Pharmaceuticals, Inc.), TMC435350 (also referred to as TMC435, Medivir/Tibotec), VBY-376 and VBY-106 (Virobay), VP50406 (ViroPharma, Inc.), VX-500 (Vertex Pharmaceuticals, Inc.), VX 550 (Vertex Pharmaceuticals, Inc.) or VX-813 (Vertex Pharmaceuticals, Inc.).

Another specific non-limiting example of other therapies that may be used in combination with a Compound for treating a viral infection include the HCV NS4a protease cofactor inhibitor or HCV NS4a protease cofactor inhibitor ACH-806 (also referred to as GS-9132, Achillion/Gilead) or ACH-1095 (also known as GS-9525, Gilead/Achillion).

Another specific non-limiting example of other therapies that may be used in combination with a Compound for treating a viral infection include the nucleoside or non-nucleoside NS5b polymerase inhibitor A-837093 (Abbott Laboratories), A-848837 (Abbott Laboratories), ABT-333 (Abbott Laboratories), ABT-072 (Abbott), AG-021541 (Pfizer Pharmaceuticals), ANA598 (Anadys Pharmaceuticals, Inc.), BILN-1941 (Boehringer Ingelheim Pharma), GL-59728 (Genelabs), GL-60667 (Genelabs), GS-6620 (Gilead), GS-9190 (Gilead), GSK-625433 (GlaxoSmithKline), HCV-796 (Wyeth/Viropharma, Inc.), HCV-896 (ViroPharma, Inc.), IDX102 (Idenix Pharmaceuticals, Inc.), IDX184 (Idenix Pharmaceuticals, Inc.), IDX375 (Idenix Pharmaceuticals, Inc.), JDK-003 (Akros Pharmaceuticals), MK-0608 (Merck), MK-3281 (Merck), NM107 (active moiety of valopicitabine, Idenix/Novartis), PF-00868554 (also referred to as PF-868554 or PF-868,554, Pfizer Pharmaceuticals), PSI-6130 (Pharmasset), PSI-7851 (Pharmasset), PSI-7977 (Pharmasset), R1626 (a prodrug of R1479, Roche Pharmaceuticals), R7128 (a prodrug of PSI-6130, Pharmasset/Roche Pharmaceuticals), valopicitabine (also referred to as NM-283, Idenix/Novartis), VBY-708 (Virobay), VCH-222 (Virochem/Vertex), VCH-759 (Virochem/Vertex), VCH-916 (Virochem/Vertex) or XTL-2125 (also referred to as BC2125, XTL Biopharmaceuticals, Ltd.).

Another specific non-limiting example of other therapies that may be used in combination with a Compound for treating a viral infection include the NS4b inhibitor anguizole (Genelabs/GSK/Viropharma, Inc.), clemizole (Stanford University) or Compound A (BMS) or Apath NS4B inhibitors.

Another specific non-limiting example of other therapies that may be used in combination with a Compound for treating a viral infection include the N55a inhibitor A-689 (also referred to as AZD7295, Arrow Therapeutics, Ltd./AstraZeneca), A-831 (also referred to as AZD2836, Arrow Therapeutics, Ltd./AstraZeneca), BMS-790052 (Bristol-Myers Squibb), GS-5885 (Gilead), ACH-2928 (Achillion), PPI-461 (Presidio), PPI-1301 (Presidio), DEP-239 (Enanta).

Another specific non-limiting example of other therapies that may be used in combination with a Compound for treating a viral infection include the IRES inhibitor steroid mifepristone (also referred to as VGX-410C, VGX Pharmaceuticals), an antisense oligonucleotide ISIS-14803 (Isis Pharmaceuticals), a ribozyme such as HEPTAZYME®, (a synthetic ribozyme, Ribozyme Pharmaceuticals, Inc.), a RNAi such as TT033 (Benitec/Tacere Bio/Pfizer) or SIRNA-034 (Sirna Therapeutics), a miRNA such as SPC3649 (LNA-antimiR™-122 brand, Santaris Pharma) or an anti-miR-122 miRNA (Regulus Therapeutics) or siRNA.

Another specific non-limiting example of other therapies that may be used in combination with a Compound for treating a viral infection include the p7 inhibitor BIT225 (Biotron Limited), the viral entry inhibitor ITX5061 (iTherX Pharmaceuticals, Inc.), PRO206 (Progenics), an SP-30 entry inhibitor (Samaritan Pharmaceuticals) or a broad spectrum entry inhibitor such as REP 9AC (an amphipathic DNA polymer, REPLICor, Inc.).

Another specific non-limiting example of other therapies that may be used in combination with a Compound for treating a viral infection include ribavirin (VIRAZOLE® and VILONA® brands, ICN Pharmaceuticals), ribavirin for oral administration (REBETOL® brand, Merck/Schering-Plough Corporation), ribavirin tablets (COPEGUS® brand, Roche Pharmaceuticals), ribavirin capsules (RIBASPHERE® brand, Three Rivers Pharmaceuticals, LLC),

Another specific non-limiting example of other therapies that may be used in combination with a Compound for treating a viral infection include the ribavirin analogue levovirin (L-isomer of ribavirin, Valeant Pharmaceuticals), R1518 (a prodrug of levovirin, also referred to as levovirin valinate, Roche Pharmaceuticals) or taribavirin (an oral prodrug of ribavirin, also referred to as viramidine, Valeant Pharmaceuticals).

Another specific non-limiting example of other therapies that may be used in combination with a Compound for treating a viral infection include the non-pegylated interferon (optionally administered with ribavirin) interferon alfa-2a (ROFERON®-A brand, Roche Pharmaceuticals), interferon alfa-2b (INTRON® A brand, Merck/Schering-Plough Corporation), interferon alfa-2c (BEROFOR® brand, Boehringer Ingelheim), interferon-alpha variant GEA007.1 (GenOdyssee SA), interferon-alpha for low dose oral administration (Amarillo Biosciences, Inc./CytoPharm, Inc.), interferon-alpha for oral administration (BELEROFON® brand, Nautilus Biotech), long-acting interferon-alpha (LOCTERON® brand, also referred to as BLX-883, Biolex Therapeutics/OctoPlus), long-acting albuminfusion interferon alfa-2b (ALBUFERON® brand, also referred to as albinterferon alfa-2b, Human Genome Sciences), purified multi-subtype human leukocyte interferon-alpha (MULTIFERON® brand, Swedish Orphan International), interferon beta-1a (REBIF brand, Merck Serono), interferon omega (also referred to as leukocycte (II) interferon, Intarcia Therapeutics), interferon omega (VIRBAGEN OMEGA® brand, Virbac), interferon omega (OMEGA INTERFERON® brand, Biomedicines), consensus interferon (INFERGEN® brand, also referred to as interferon alfacon-1, Three Rivers Pharma), medusa interferon (MEDUSA INTERFERON® brand, Flamel Technologies).

Another specific non-limiting example of other therapies that may be used in combination with a Compound for treating a viral infection include the pegylated interferon (optionally administered with ribavirin) Peginterferon alfa-2a (PEGASYS® brand, Roche Pharmaceuticals), Peginterferon alfa-2b (PEGINTRON® brand, Merck/Schering-Plough Corporation), Peginterferon alfacon-1 (pegylated form of interferon alfacon-1, also referred to as PEG-Alfacon, InterMune), Peg-Interferon Lambda IL-29 (Zymogenetics/Bristol-Myers Squibb).

Another specific non-limiting example of other therapies that may be used in combination with a Compound for treating a viral infection include the TLR agonist ANA773 (Anadys Pharmaceuticals, Inc.), a TLR-7 agonist selected from isatoribine (also referred to as ANA245, Anadys Pharmaceuticals, Inc.), ANA-971 (a prodrug of TLR-7 agonist isatoribine, Anadys Pharmaceuticals, Inc.), ANA975 (a prodrug of TLR-7 agonist isatoribine, Anadys Pharmaceuticals, Inc.), a TLR9 agonist selected from IMO-2125 (Idera Pharmaceuticals), a TLR9 agonist (Actilon brand, Coley), a cyclophilin B inhibitor selected from Debio 025 (Debiopharm Group) or SCY-635 (Scynexis) or a cyclosporin A analog selected from NIM811 (Novartis), a pancaspase inhibitor selected from PF-03491390 (also referred to as IDN-6556, Pfizer Pharmaceuticals), an interleukin-7 immunomodulator selected from CYT107 (Cytheris SA), NOV-205 (Novelos Therapeutics), oglufanide disodium (Implicit Bioscience) or thymosin alpha 1 (also referred to as thymalfasin, ZADAXIN® brand, SciClone Pharmaceuticals), a immunomodulator/antiinflammatory selected from NOV205 (Novelos Therapeutics, Inc.), an antiinflammatory selected from CTS-1027, a matrix metalloproteinase selected from a (MMP) inhibitor (Conatus) or CF102, an A3AR agonist (Can-Fite BioPharma, Ltd.), an antiinflammatory/antifibrotic selected from mitoquinone (MitoQ® brand, Antipodean Pharmaceuticals) or PYN17 (Phynova), a broad spectrum immune stimulator selected from SCV-07 (SciClone), an immune regulator selected from ECH18 (Enzo BioChem/Therapeutics), an antifibrotic selected from JKB-122 (Jenken Biosciences), a tumor necrosis factor inhibitor antifibrotic selected from ENBREL® brand (Wyeth), a phospholipid antifibrotic for oral administration selected from IP-501 (Indevus Pharmaceuticals), a hemopurifier (Aethlon Medical), an IMPDH inhibitor selected from merimepodib (also referred to as VX-497, Vertex Pharmaceuticals, Inc.), a glucosidase inhibitor selected from celgosivir, an alpha-glucosidase I inhibitor selected from MX-3253 (Migenix), a HCV therapeutic vaccine selected from a DNA vaccine (ChronVac-C® brand, Inovio/Tripep AB), a MVA virus vaccine carrying and expressing HCV non-structural proteins (NS3, NS4 and NS5b) selected from TG4040 (Transgene) or (Inovio/Tripep AB), an antiviral vaccine selected from GNI-103 (GENimmune), a virosome-based combination vaccine of synthetic HCV peptide antigens (Pevion Biotect), an E1 vaccine (Innogenetics), a HCV E1/E2/MF59 vaccine (Chiron/Novartis), a vaccine selected from CSL123 (Chiron/CSL), a targeted molecular immunogen vaccine selected from GI-5005 (GlobeImmune), a vaccine having a combination of five synthetic peptides selected from IC-41 (Intercell AG/Novartis), an antiviral vaccine (AMANTADINE® brand, Endo Labs), a monoclonal antibody selected from 170® (also referred to as HCV-AB^{XTL}68 or HCV-AB, Biochem Therapeutics/OSI Pharmaceuticals), an immune globulin polyclonal antibody selected from intravenous human immune globulin (CIVACIR® brand, NABI), a humanized Y-90 labeled antibody (Immunomedics, Inc.) an anti-PD1 antibody selected from MDX-1106 (also referred to as ONO-4538, Medarex, Inc./Ono Pharmaceutical), an anti-CD20 monoclonal antibody (RITUXIMAB® brand, Genentech), a monoclonal antibody selected from XTL-6865 or XTL-002 (XTL Biopharmaceuticals, Ltd.), a HIV fusion inhibitor selected from enfuvirtide (FUZEON® brand, Trimeris/Roche Pharmaceuticals), an anti-phospholipid therapy selected from bavituximab (formerly TARVACIN® brand, Peregrine Pharmaceuticals, Inc.), a protein therapeutic or interferon replacement agent selected from oligoadenylate synthetase stimulant CB-183,872 (Cubist Pharmaceuticals, also referred to as IB657 from Illumigen Biosciences), a botanical selected from an antiviral botanical extract PYN18 (Phynova) or a non-specific pharmaceutical selected from the cholesterol-lowering agent fluvastatin (Oklahoma University Health Sciences Center), atorvastatin (Okayama University, Japan), lovastatin (Okayama University, Japan) or simvastatin (Okayama University, Japan), a thiazolide analog selected from nitazoxanide (ALINIA™ brand, Romark Pharmaceuticals), photo-sensitized methylene blue (SUVUS® brand, Bioenvision), a synthetic phytochemical selected from KPE02003002 (Kemin Pharma) or KPE00001133 (Kemin Pharma), an antiviral agent selected from CB5300 (Canopus BioPharma, Inc.) or a tyrosine phosphatase inhibitor selected from sodium stibogluconate (LENOCTA™ brand, VioQuest Pharmaceuticals).

Another specific non-limiting example of other therapies that may be used in combination with a Compound for treating a viral infection include the non-specific pharmaceutical histamine dihydrochloride (CEPLENE® and MAXAMINE® brands, Maxim Pharmaceuticals), an immunosuppressive agent selected from mycophenolate mofetil (Roche Pharmaceuticals), mycophenolic acid (Roche Pharmaceuticals), or α1-antichymotrypsin.

6.9 Kits

Provided herein is a pharmaceutical pack or kit comprising one or more containers filled with a Compound or pharmaceutical composition thereof. Additionally, one or more other therapies useful for the treatment of a viral infection, or other relevant agents can also be included in the pharmaceutical pack or kit. Also provided herein is a pharmaceutical pack or kit comprising one or more containers filled with one or more of the ingredients of the pharmaceutical compositions described herein. Optionally associated with such kits can be a notice in the form prescribed by a governmental agency regulating the manufacture, use or sale of pharmaceuticals or biological products, which notice reflects approval by the agency of manufacture, use or sale for human administration.

### 7. EXAMPLE: FORMULATION OF COMPOUND #10

Compound #10 can be formulated by those skilled in the art using known methods, including those set forth in International Publication Nos. WO2005/089764, WO2006/113703, WO2008/127715, WO2008/127714, and WO 2010/138758, each of which is incorporated by reference herein in its entirety.

### 8. EXAMPLE: FORMULATION OF COMPOUND 1205

Compound 1205 is bioavailable in vivo when administered in an aqueous suspension. It is anticipated that Compound 1205 can be clinically administered via a solid-dosage form. For all the studies outlined herein, Compound 1205 was lyophilized prior to formulation to minimize batch-to-batch variations in particle-size.

The compound was dissolved in acetonitrile at a concentration of 15 mg/mL. An equal amount of water was added to bring the final concentration to 7.5 mg/mL in a 1:1 acetonitrile/water solution (v/v). The solution was frozen on the shelf of the freeze dryer for a minimum of 3 hours and then lyophilized. The resulting solid was determined to be amorphous by differential scanning calorimetry and polarized light microscopy. Suspensions were prepared by the addition of 0.5% HPMC with 1% Tween-80, followed by stirring and homogenization for 2 minutes.

The examples that follow demonstrate that the Compounds tested can inhibit the pathological production of human VEGF, and suppress edema, inflammation, pathological angiogenesis and tumor growth tumor growth. Compounds tested have been shown to inhibit the pathological production of human VEGF by multiple human tumor cells and/or human tumors in animal models with pre-established human tumors.

### 9. EXAMPLE: COMPOUND PHARMACODYNAMICS

9.1 Cell Cycle Delay

9.1.1 Cell Based Assays

9.1.1.1Compound #10 and Compound 1205 Provoke a Late G₁/Early S-Phase Cell Cycle Delay

This example demonstrates that a Compound induces a cell cycle delay at the G₁/S-phase border.

Experimental Design.

During in vitro evaluations of Compound #10 and Compound 1205 effects on VEGF expression, an examination of the effect on tumor cell cycling was performed. HT1080 cells were incubated under normoxic conditions (21% oxygen) for 18 hours with vehicle (0.5% DMSO) alone, or with a range of concentrations of Compound #10 from 0.3 nM to 100 nM, or 10 nM of Compound 1205. Compounds shown in Table 2 were incubated under normoxic conditions for 18 hours with vehicle or Compound #10 at a single dose of 100 nM. After treatment, cells were trypsinized, and stained with propidium iodide (PI) dye to measure DNA content of individual cells by flow cytometry. Output comprised histograms showing relative DNA content in 10,000 cells.

Results.

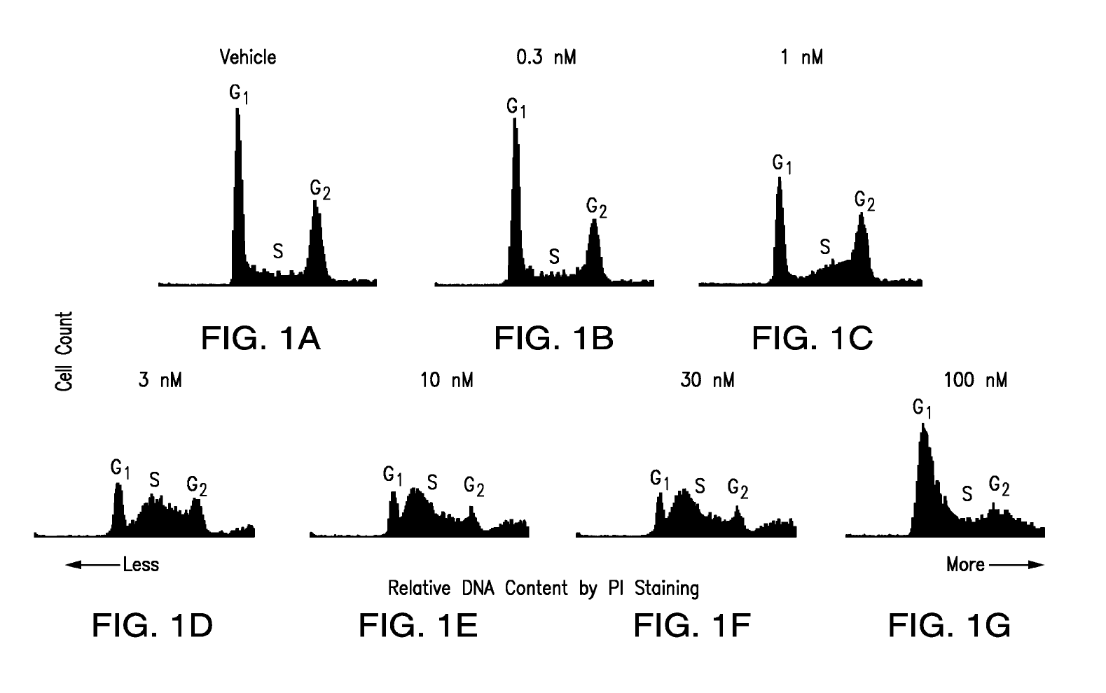

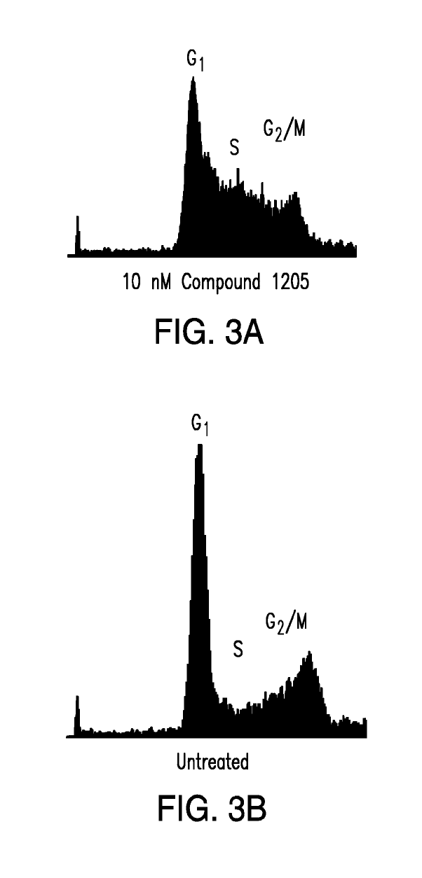

As shown in FIG. 1 and FIG. 3, Compound #10 and Compound 1205 induced a redistribution of the cycling characteristics of the cell population. An apparent dose response was observed for Compound #10. Starting at a concentration of 1 nM for Compound #10, an accumulation of cells in S phase can be observed. With higher concentrations of Compound #10, there is a progressive shift, such that a substantial proportion of the cells show a cell cycle delay at the G₁/S phase border. Concentrations of Compound #10 achieving these effects are consistent with those demonstrating inhibition of VEGF production (FIG. 1).

For additional Compounds shown in Table 2, the test results are expressed as the percentage of cells in the S-phase compared to a DMSO control (17.3% cells in S-Phase). While compounds which cause greater than 20% of the cells to accumulate in S-phase at 100 nM are considered active, a larger percentage of cells may be accumulated in S-phase at lower doses depending on the Compound, as shown in FIG. 1 for example.

TABLE 2

| | % |
|---|---|
| | Cells |
| | In |
| | S- |
| Compound | Phase |
| DMSO (Control) | 17.3 |
| 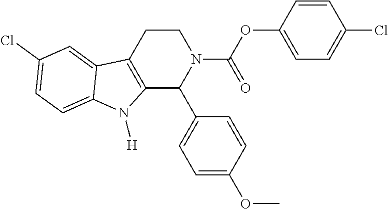 [SMILES: COc1ccc(C2c3[nH]c4ccc(Cl)cc4c3CCN2C(=O)Oc2ccc(Cl)cc2)cc1] | 15.3 |
| 10 | |
| 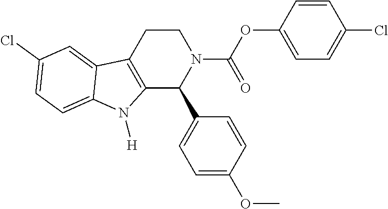 [SMILES: COc1ccc([C@H]2c3[nH]c4ccc(Cl)cc4c3CCN2C(=O)Oc2ccc(Cl)cc2)cc1] | 26.1 |
| #10 | |
| 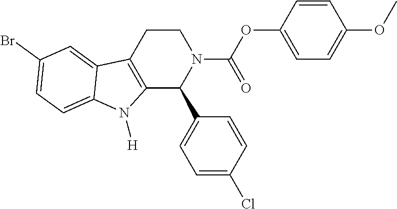 [SMILES: COc1ccc(OC(=O)N2CCc3c([nH]c4ccc(Br)cc34)[C@@H]2c2ccc(Cl)cc2)cc1] | 26.4 |
| #332 | |
| 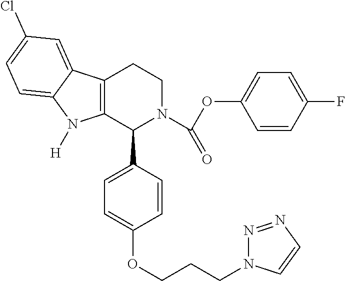 [SMILES: O=C(Oc1ccc(F)cc1)N1CCc2c([nH]c3ccc(Cl)cc23)[C@@H]1c1ccc(OCCCn2ccnn2)cc1] | 25.7 |
| 1732 | |
| 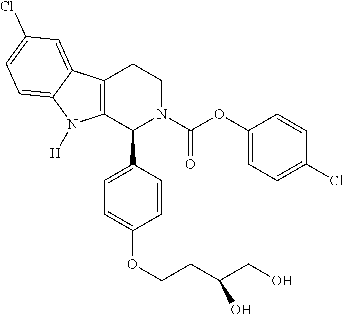 [SMILES: O=C(Oc1ccc(Cl)cc1)N1CCc2c([nH]c3ccc(Cl)cc23)[C@@H]1c1ccc(OCC[C@H](O)CO)cc1] | 20.0 |
| 1205 | |
| 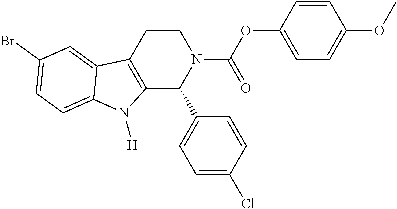 [SMILES: COc1ccc(OC(=O)N2CCc3c([nH]c4ccc(Br)cc34)[C@H]2c2ccc(Cl)cc2)cc1] | 16.5 |
| 1733 | |
| 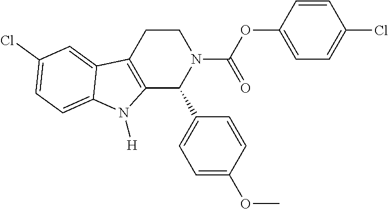 [SMILES: COc1ccc([C@@H]2c3[nH]c4ccc(Cl)cc4c3CCN2C(=O)Oc2ccc(Cl)cc2)cc1] | 16.8 |
| 1734 | |
| 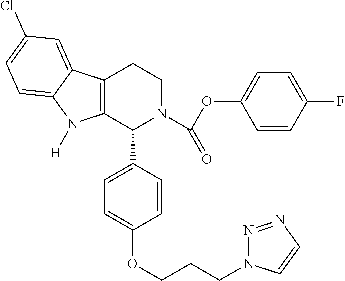 [SMILES: O=C(Oc1ccc(F)cc1)N1CCc2c([nH]c3ccc(Cl)cc23)[C@H]1c1ccc(OCCCn2ccnn2)cc1] | 16.4 |
| 1735 | |
| 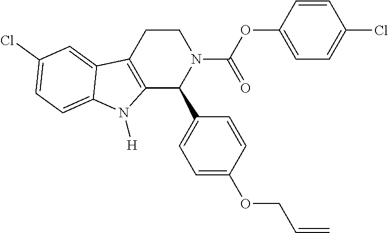 [SMILES: C=CCOc1ccc([C@H]2c3[nH]c4ccc(Cl)cc4c3CCN2C(=O)Oc2ccc(Cl)cc2)cc1] | 17.2 |
| 1159 | |
| 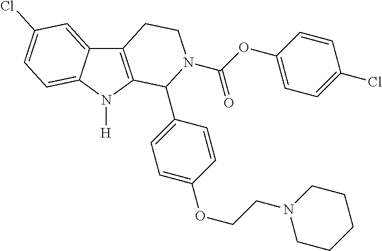 [SMILES: O=C(Oc1ccc(Cl)cc1)N1CCc2c([nH]c3ccc(Cl)cc23)C1c1ccc(OCCN2CCCCC2)cc1] | 16.8 |
| 1736 | |
| 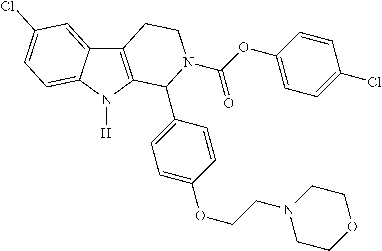 [SMILES: O=C(Oc1ccc(Cl)cc1)N1CCc2c([nH]c3ccc(Cl)cc23)C1c1ccc(OCCN2CCOCC2)cc1] | 16.4 |
| 331 | |
| 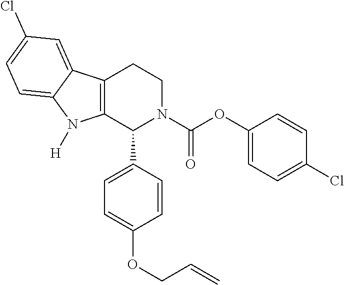 [SMILES: C=CCOc1ccc([C@@H]2c3[nH]c4ccc(Cl)cc4c3CCN2C(=O)Oc2ccc(Cl)cc2)cc1] | 17.9 |
| 1737 | |
| 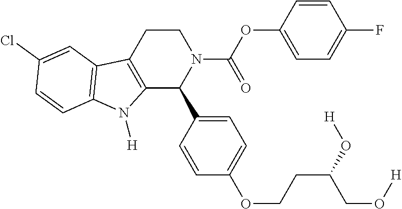 [SMILES: O=C(Oc1ccc(F)cc1)N1CCc2c([nH]c3ccc(Cl)cc23)[C@@H]1c1ccc(OCC[C@H](O)CO)cc1] | 20.6 |
| 1293 | |
| 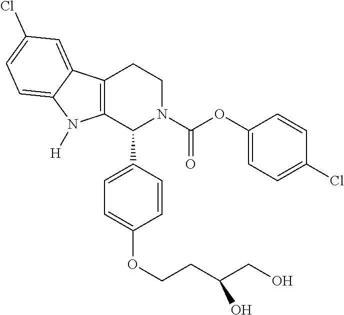 [SMILES: O=C(Oc1ccc(Cl)cc1)N1CCc2c([nH]c3ccc(Cl)cc23)[C@H]1c1ccc(OCC[C@H](O)CO)cc1] | 17 |
| 1738 | |

9.1.1.2 The Effect of Compound #10 on the Cell Cycle is Reversible

This example demonstrates that the effect of Compound #10 on cell cycle delay is reversible.

Experimental Design.

HT1080 cells were incubated under normoxic conditions (21% oxygen) for 14 hours with Compound #10 (100 nM) or with vehicle (0.5% DMSO) alone. Compound #10 was then washed out of the cultures and cells were harvested and analyzed by PI staining and flow cytometry (as described in Section 9.3.1.1) at 0, 2, 5, 8, and 26 hours after discontinuation of treatment.

Results.

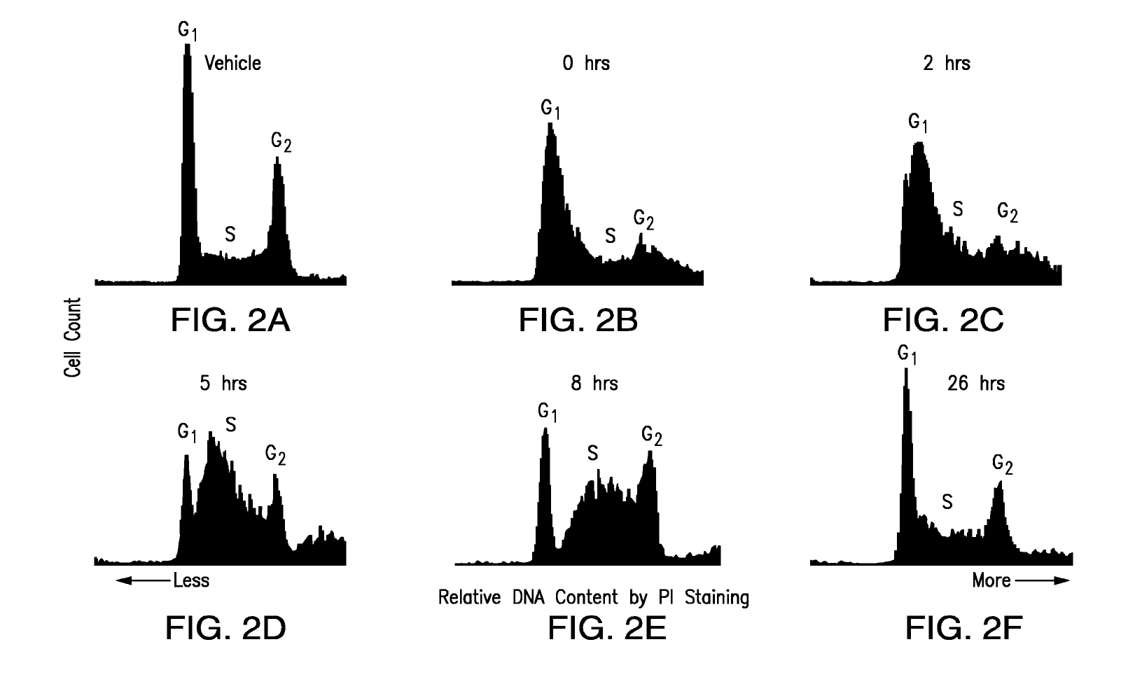

As shown in FIG. 2, treatment with Compound #10 caused the expected increase in the proportion of cells in late G₁/S phase of the cell cycle (Time 0). At 2 hours after Compound #10 removal, a shift was beginning to occur; however, a large percentage of the cells remained delayed in G₁/S. By 5 to 8 hours, cells were clearly redistributing. By 26 hours after Compound #10 washout, the cells had resumed normal cycling.

9.1.1.3 The Kinetics of S-Phase Transit Employing BrdU Incorporation into DNA

This example demonstrates the rate and number of cells transiting the S-phase of the cell cycle.

Experimental Design.

HT 1080 cells are exposed to BrdU (bromodeoxyuridine, a synthetic nucleoside that is an analogue of thymidine and is incorporated into DNA during the S phase of cell division) (FITC BrdU Flow Kit, BD Pharmingen catalog #552598). Cells are grown and treated as described in Section 9.3.1.3 above with the exception that one hour prior to harvesting by trypsinization, BrdU (final concentration 1 μM) is added to each culture for 1 hour. Cells actively replicating DNA during this brief time incorporate the BrdU into the DNA, which can then be quantified. BrdU content is quantified using the FITC BrdU Flow Kit as instructed by the manufacturer. The process includes fixation (paraformaldehyde) and DNA staining with 7-AAD (7-amino-actinomycin D) followed by incubation with a fluoro-tagged anti-BrdU antibody that specifically recognizes BrdU incorporated into DNA. Dual channel FACS analysis permits assessment of both the DNA content of individual cells and the rate of transit across the S-phase, which is assessed based upon BrdU incorporation over the one hour treatment period.

Results.

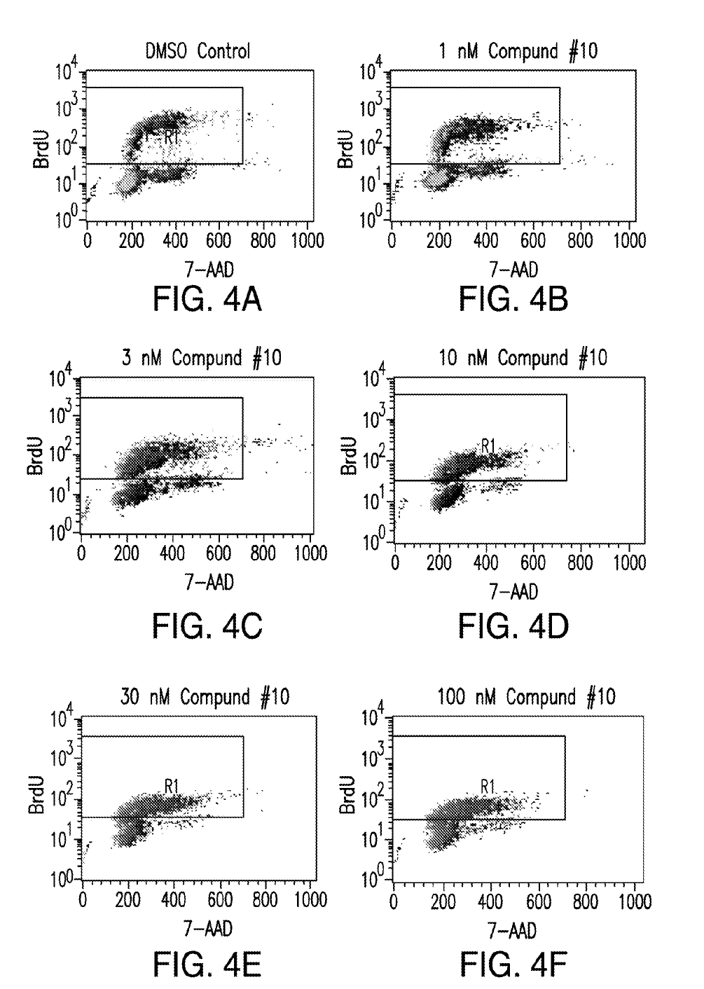

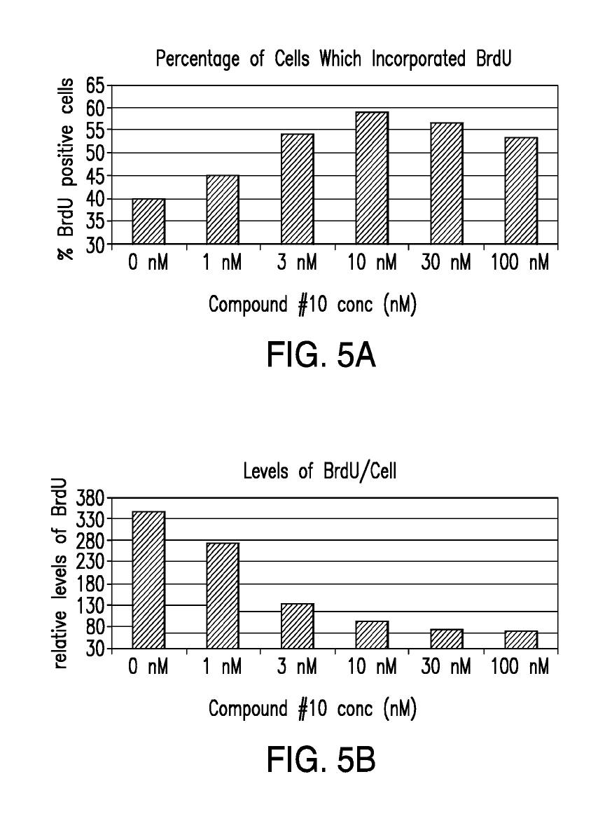

FIG. 4 indicates that an 18-hour treatment with increasing doses of Compound #10 causes a net increase in the percentage of cells residing in S-phase; however, individual cells incorporated less BrdU during the one-hour treatment period compared to DMSO control cells. The percentage of cells incorporating BrdU and the relative level of BrdU at each Compound #10 concentration is shown in FIG. 5. These results suggest that Compound #10 slows the transit of cells through the S-phase of the cell cycle.

9.1.1.4 The Effect of Compound #10 on the 3-Dimensional Growth of HT 1080 Cells

This example demonstrates the effect of a Compound provided herein on the 3-dimensional growth of HT1080 cells.

Experimental Design.

HT1080 cells grown as a monolayer were trypsinized and seeded onto a 0.75% agar noble base to prevent the cells from attaching to the bottom of the tissue culture plate and to allow/promote the cells to self-adhere and grow as 3-dimensional spheroids. After 4 days the spheroids were established and the liquid growth medium was replaced with medium containing either 0.5% DMSO vehicle, or 10 nM or 50 nM of Compound #10 with 0.5% DMSO vehicle. The cells were incubated for 22 and 45 hours at 37° C., in the presence of a 10% CO₂ atmosphere. Spheroids were visually checked daily for morphological changes and a medium was replenished two times per week. At 22 and 45 hours after exposure to Compound #10, BrdU was added to a subset of the wells designated for FACS analysis and then returned to the incubator for 3 hours to permit cells synthesizing DNA (i.e. cells in S-phase) to incorporate the BrdU into the nascent strands of DNA. These pulse labeled spheroids were then harvested, washed and trypsinized (triple action solution, Gibco), pelleted and prepared for FACS analysis with a FITC BrdU Flow Kit, (BD Pharmingen). Cells were fixed and permeabilized with paraformadehyde and DNA stained with 7-AAD followed by incubation with an antibody which specifically recognizes BrDU incorporated into DNA. As described in Section 9.3.1.4. Cells were analyzed and sorted by 7-AAD signal (DNA content) to determine cell cycle phase, and BrdU content (percent actively synthesizing DNA).

Results.

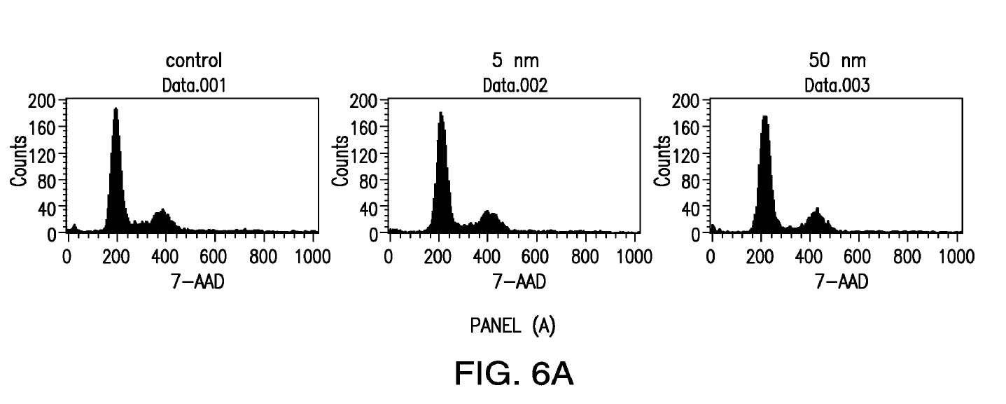

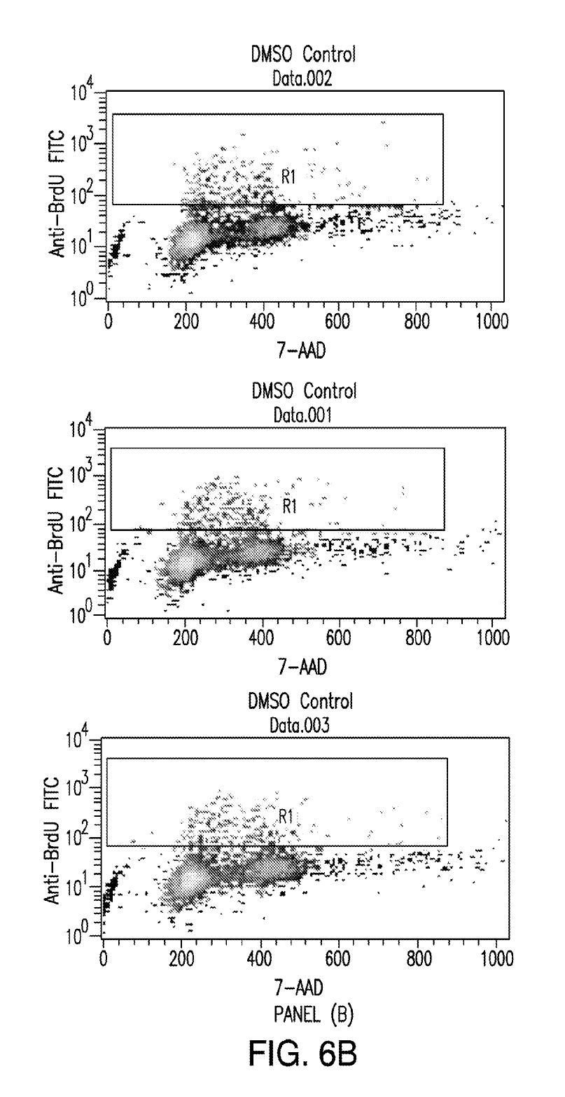

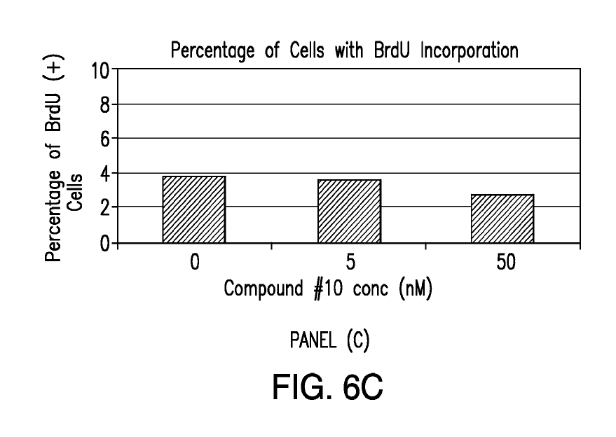

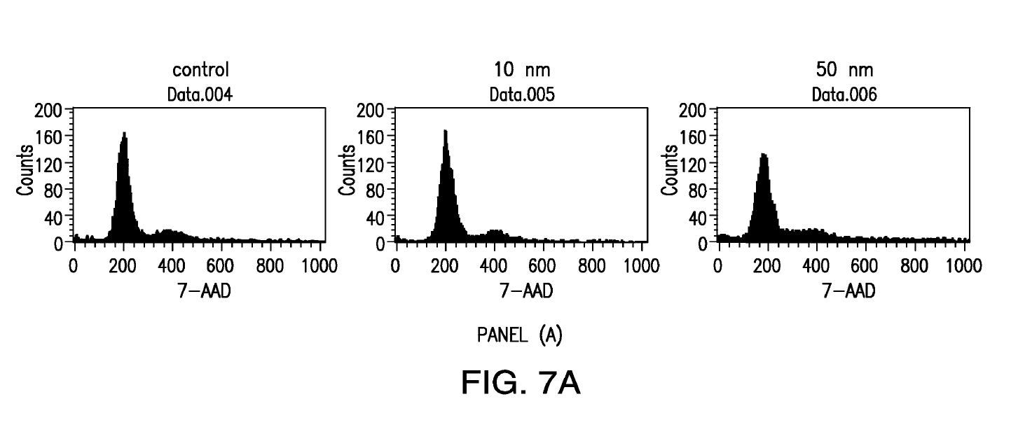

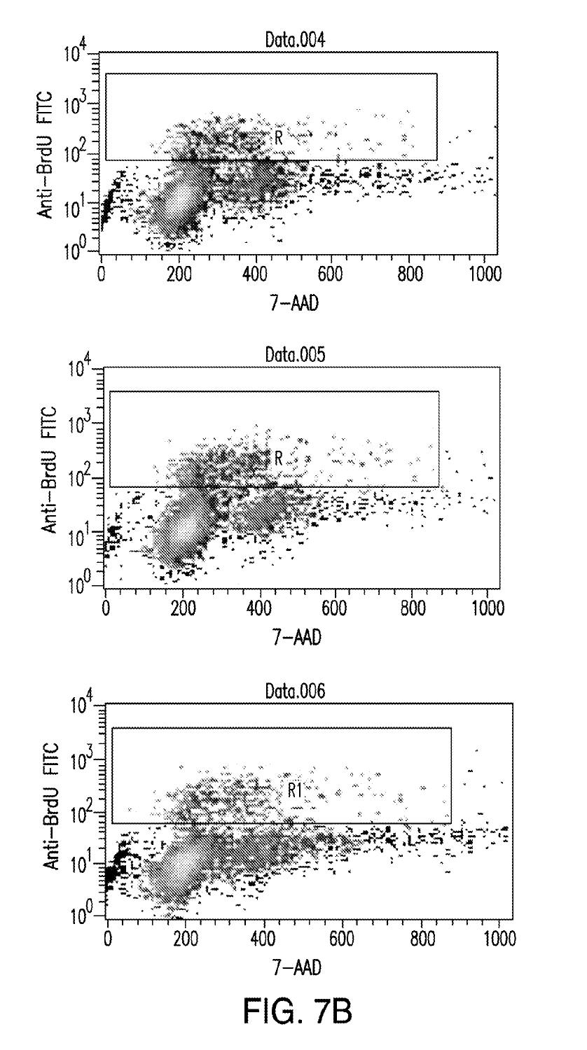

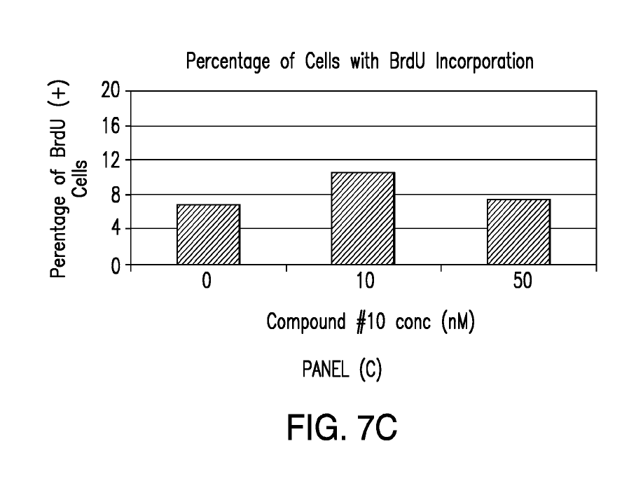

HT1080 spheroids prepared as above were treated with a Compound provided herein for 24 (FIG. 6) or 48 hours (FIG. 7). FIG. 6 and FIG. 7 show: (A) a histogram of DNA content demonstrating that the cell cycle distribution is not affected by exposure to the Compound provided herein; (B) BrdU quantification indicating the fraction of cells actively synthesizing DNA; and (C) a graphical representation of the percentage of cells that incorporated BrdU (i.e., the cells in S-phase), indicating that the percentage is not significantly altered by compound #10 treatment.

Spheroids, prepared as above, were treated with either vehicle alone (0.5% DMSO v/v final) added to the media or a Compounds provided herein (10 nM or 50 nM final concentration) in media to which vehicle has been added. The cells were photographed on day 5 of treatment to assess any gross morphological differences caused by exposure to Compound #10. Spheroids from all treatment groups looked indistinguishable from one another (data not shown). In addition, spheroids maintained in the presence of Compound #10 provided herein for three weeks also display no obvious morphological changes (data not shown).

9.2 Compound 1205 Provokes a Late G₁/Early S-Phase Cell Cycle Delay

This example demonstrates that Compound 1205 provokes a cell cycle delay at the G₁/S-phase border.

Experimental Design.

The in vitro evaluation of Compound 1205 effects on tumor cell cycling was performed. HT1080 cells were incubated under normoxic conditions (21% oxygen) for 18 hours with vehicle (0.5% DMSO) alone, or with 10 nM of Compound 1205. After treatment, cells were trypsinized, and stained with propidium iodide (PI) dye to measure DNA content of individual cells by flow cytometry. Output comprised histograms showing relative DNA content in 10,000 cells.

Results.

As shown in FIG. 5, Compound 1205 induced a redistribution of the cycling characteristics of the cell population.

### 10. EXAMPLE: INHIBITION OF VIRAL REPLICATION

Viral Replication Assays:

A person of ordinary skill in the art may test a Compound for antiviral activity using a variety of different approaches, with a representative number of selected examples as detailed below.

HCV Replicon Assay:

The lack of validated and readily accessible cell-culture whole virus infection systems and small animal models permissive for HCV replication has limited the development of new anti-HCV agents. Self-replicating genomic and subgenomic HCV systems, termed HCV replicons, have been described and have been widely used to assess the efficacy of anti-HCV inhibitors (see Blight K J, et al., 2000, Efficient initiation of HCV RNA replication in cell culture. Science 290:1972-1974; Blight K J, et al., 2002, Highly permissive cell lines for subgenomic and genomic hepatitis C virus RNA replication. J Virol 76:13001-13014; Ikeda M, et al., 2002. Selectable subgenomic and genome-length dicistronic RNAs derived from an infectious molecular clone of the HCV-N strain of hepatitis C virus replicate efficiently in cultured Huh7 cells. J Virol 76:2997-3006; Lohmann V, et al., 1999, Replication of subgenomic hepatitis C virus RNAs in a hepatoma cell line. Science 285:110-113; Pietschmann T, et al., 2002, Persistent and transient replication of full-length hepatitis C virus genomes in cell culture. J Virol 76:4008-4021; and, Pietschmann T, et al., 2001, Characterization of cell lines carrying self-replicating hepatitis C virus RNAs. J Virol 75:1252-1264).

U.S. Pat. No. 6,630,343 describes a bicistronic HCV 1b replicon and 2a replicon for use in testing a Compound by quantifying replicon RNA (GenBank Accession No. AJ242654) reduction and/or the Fluc reporter signal. The amount of HCV replicon RNA is determined by quantitative reverse transcription polymerase chain reaction (qRT-PCR). In some cases, a Compound is tested against the HCV replicon in a spheroid culture. Replicon-containing cells may be cultured with a Compound for up to 3 days. Interferon (IFN) α is used as a positive control.

Standard cell culture assays employing HCV subgenomic replicons showed that the Compound had an average IC₅₀ of 0.036 μM against the genotype 1b replicon and an IC₅₀ of <0.003 μM against the genotype 2a replicon. Performing a replicon assay under three-dimensional culture conditions (spheroid culture) resulted in an IC₅₀ of 0.001 μM against the genotype 1b replicon and >310 fold selectivity index. Notably, the R-enantiomer of the Compound failed to exhibit significant antiviral activity in parallel experiments.

Attempts to generate resistant HCV replicons using standard virological techniques were unsuccessful despite exposure of replicon cells to the Compound under various conditions for up to four months. Classical antivirals acting directly on viral targets typically generate robustly resistant variants within 3-4 weeks using this technique.

Poliovirus (PV) Assay:

Antiviral activity is tested against PV strain Mahoney (obtained from Dr. Eckard Wimmer, State University of New York as Stony Brook, Stony Brook, New York) in HeLa S3 cells by determining the viral RNA reduction using qRT-PCR. HeLa S3 cells are seeded onto 96 well plates at a density of 5000 cells per well and incubated in DMEM supplemented with 10% FBS and 1% penicillin-streptomycin at 37° C. under 5% CO₂ for 24 hours and then treated with a Compound at a series of test concentrations for 18 hours. The cells are then infected with PV at a multiplicity of infection of 0.1 in DMEM without FBS for 30 minutes, followed by treatment with a Compound at a series of concentrations in DMEM with 1% FBS for 20 hours. After removing supernatant and washing the cells with PBS, RNA is prepared by adding 50 μL of Cells-to-cDNA Cell Lysis Buffer (Ambion, Catalog #8723) to each well and then heating at 75° C. for 10 minutes. The cell lysate is then treated with DNase I (DNA-free™ Ambion, Catalog #1906) at 37° C. for 20 minutes and then heated at 75° C. for 5 minutes to inactivate DNase. cDNA is prepared using iScript RT kit (Bio-Rad, Catalog #170-8897). The viral cDNA is determined by qRT-PCR using a pair of primers and a probe complementary to the viral internal ribosome entry site. The IC₅₀ shown in Table 3 is calculated based on percentage of viral RNA reduction under treatment of a Compound using Prism nonlinear fit sigmoidal dose-response variable slope (GraphPad Prism Software).

Additionally, in a 24-hour assay of infected HeLa cells when the Compound was added about 16 hours pre-infection, PV was inhibited with an average IC₅₀ of 0.0006 μM. Adding the Compound at the time of infection, though, resulted in a 65-fold decrease in activity. In HT-1080 cells, the Compound inhibited PV with an average IC₅₀ of 0.0004 μM. A variant HT-1080 cell line which displayed resistance to the cell cycle effects of the Compound was generated through serial passage; in these cells the Compound inhibited PV with an average IC₅₀ of 4.7 μM, a 10.000-fold difference in activity from that observed in non-resistant cells.

Other Viral Assays

Antiviral activity of a Compound against WNV is tested in Vero cells by protection of virus induced cytopathic effects (i.e. cytoprotection measured as cell viability, IC₅₀). The effect of a Compound on inhibition of virus induced cytopathic effects is determined using MTS (CellTiter) assay.

Antiviral activity against vaccinia virus is determined in Vero E6 cells by a plaque reduction assay. For a plaque reduction assay, inhibition of viral replication is determined as a reduction in virus-induced plaque formation assessed by microscopic inspection following staining of the culture with crystal violet.

The activity against HIV-1 was tested in MT2 cells and PBMCs in cell culture by preventing virus induced cytopathic effect and measuring viral p24 protein in the culture supernatant.

TABLE 3
Activity of Compound #10 in Antiviral Assay Panel

| Virus | | Cell line | Assay | IC₅₀ (μM) | IC₉₀ (μM) | CC₅₀ (μM) |
|---|---|---|---|---|---|---|
| Vaccinia | DNA | Vero E6 | Plaque | 0.040 | 0.080 | 0.083 |
| Adenovirus | DNA | HeLa | CPE | >1 | >1 | >1 |
| | | | protection | | | |
| HSV-1 | DNA | Vero | CPE | >1 | >1 | >1 |
| | | | protection | | | |
| Influenza A | (−) RNA | MDCK | CPE | >1 | >1 | >1 |
| | | | protection | | | |
| Parainfluenza | (−) RNA | Vero | CPE | 0.029 | 0.044 | >1 |
| | | | protection | | | |
| RSV | (−) RNA | Vero | CPE | 0.25 | ≧0.16 | >1 |
| | | | protection | | | |
| Yellow Fever | (+) RNA | HeLa | CPE | >1 | >1 | >1 |
| | | | protection | | | |
| Dengue 2 | (+) RNA | Vero E6 | CPE | >1 | >1 | 0.70 |
| | | | protection | | | |
| WNV | (+) RNA | Vero | CPE | 0.067 | 0.28 | >1 |
| | | | protection | | | |
| PV | (+) RNA | HeLa | qRT-PCR | 0.00057 | 0.0028 | >1 |
| HIV-1 | Retro | MT-2 | CPE | 0.022 | NA | 0.0041 |
| | | | protection | | | |
| HIV-1 | Retro | PBMCs | p24 ELISA | >1 | NA | 0.68 |

Results.

As shown in Table 3, the Compound has inhibitory activity against a diverse panel of RNA viruses in vitro. At the doses tested in the human or monkey cell lines tested, the Compound did not inhibit the two DNA viruses adenovirus and herpes simplex virus-1 (HSV-1). At the doses tested in the human or monkey cell lines tested, the Compound was inactive against the two RNA viruses dengue and yellow fever. However, the Compound displayed potent activity against the three RNA viruses: parainfluenza virus, RSV and WNV in the cell lines tested. The Compound did not exhibit any selective inhibition of influenza virus when grown in the canine kidney cell line tested. The broad-spectrum activity of the Compound was demonstrated by its inhibition of both plus-strand (PV, HCV, WNV) and minus-strand (RSV, parainfluenza) RNA viruses. No antiviral activity was detected for the R-enantiomer of the Compound.

### 11. EXAMPLE: COMPOUND #10 FOR THE TREATMENT OF HCV

A Phase 2a Study to Assess the Activity, Safety, and Phamacokinetics of Compound #10 in Patients with Chronic Active Hepatitis C

Background: During nonclinical profiling of Compound #10, it has also been noted that the compound has potent in vitro inhibitory activity against a number of RNA viruses, including HCV, as determined in replicon systems. Evidence that Compound #10 may act via a cellular target to suppress HCV replication supports clinical assessment of Compound #10 as a novel therapy for patients with HCV infection.

Nonclinical safety testing further supports clinical development of Compound #10. Safety pharmacology studies show no adverse off-target effects. Toxicology studies in rats and dogs through 28 days indicate good tolerability at doses and exposures in excess of therapeutic nonclinical doses. Genotoxicity studies show no evidence of genotoxic effects.

Initial clinical evaluation of Compound #10 was performed in healthy volunteers. Results from a Phase 1a single-dose study indicate that Compound #10 can be administered with acceptable safety in healthy subjects at doses through 3 mg/kg (˜210 mg) (the highest single dose tested). Similarly, results from a subsequent Phase 1a, 7 day, multiple-dose study indicate that Compound #10 can be administered with acceptable safety in healthy subjects at doses through 1.2 mg/kg/dose 2 times per day (BID) (˜168 mg/day) and 1.6 mg/kg/dose 3 times per day (TID) (˜336 mg/day) (the highest dose tested).

In addition to the data available from evaluation of Compound #10 in healthy volunteers, additional safety data have been obtained from ongoing Phase 1b and Phase 2 studies of Compound #10 in patients with neoplasia, including a Phase 1b study in women with metastatic breast cancer, a Phase 1b study in patients with various advanced cancers, a Phase 1/2 study in patients with HIV-associated Kaposi sarcoma, and a Phase 2 study in patients with neurofibromatosis type 2. To date, doses of 0.3 mg/kg/dose (˜20 mg/dose) (n=6), 0.6 mg/kg/dose (˜40 mg/dose) (n=9), 1.2 mg/kg/dose (˜80 mg/dose) (n=12) BID have been tested for at least 4 weeks. In addition, 100 mg/dose BID (n=52), 100 mg/dose TID (n=6), 120 mg/dose TID (n=3), 160 mg/dose TID (N=3), and 200 mg/dose TID (n=3) have been tested for at least 6 weeks of continuous treatment. One patient has received Compound #10 treatment at 100 mg BID for >20 months. In general, the evaluation data has shown that Compound #10 has been generally well tolerated; adverse events have been infrequent, usually Grade 1 or 2 in severity, and not usually considered to be Compound #10-related.

Primary Objectives:

To determine whether Compound #10 provides pharmacological effect in HCV as measured by serum HCV-RNA viral load.

Primary Endpoint:

Change in serum HCV viral load during 14 days of Compound #10 treatment and subsequent 14-day follow-up period as determined by a quantitative assay.

Secondary Objectives:
1. To evaluate the effect of Compound #10 on additional markers of disease activity.
2. To assess Compound #10 effects on circulating angiogenic and inflammatory cytokines in subjects with HCV infection.
3. To assess Compound #10 plasma exposure in subjects with HCV infection.
4. To characterize the safety profile of Compound #10 in subjects with HCV infection.
5. To determine compliance with Compound #10 administration.

Secondary Endpoints:
1. Change in serum aminotransferase values during 14 days of Compound #10 treatment and subsequent 14-day follow-up period.
2. Change in serum and plasma values of circulating angiogenic/inflammatory cytokines (eg, VEGF, interleukin-6 [IL-6], osteopontin)
3. Pharmacokinetic (PK) parameters, eg, time to maximum plasma concentration (Tₘₐₓ), maximum concentration (Cₘₐₓ), concentration at 24 hours (C₂₄), area under the concentration-time curve (AUC), terminal elimination half-life (t_{1/2}) based on Compound #10 plasma concentrations as assessed by a validated bioanalytical method.
4. Overall safety profile characterized by type, frequency, severity, timing, and relationship to Compound #10 of any adverse events, laboratory abnormalities, or electrocardiogram (ECG) abnormalities.
5. Study drug compliance as assessed by a subject daily diary and quantification of used and unused study drug

Study Design:

This is a Phase 2a, single-center, open-label, multiple-dose, activity, safety, and PK study of Compound #10 in patients with chronic active HCV infection who have experienced a relapse to standard of care HCV treatment. This study will be conducted at a specialized research unit experienced in the conduct of clinical trials in patients with HCV infection.

Approximately 12 subjects will be enrolled over a projected recruitment period of ˜2 months. This study will enroll 2 cohorts, 1 comprising ˜6 subjects who have relapsed after a standard course of PEG-IFN/ribavirin treatment and 1 comprising ˜6 subjects who have received no prior anti-HCV treatment. Relapse is defined as the occurrence of a positive HCV-RNA test during or after a standard course of PEG-IFN/ribavirin treatment that was associated with a negative HCV-RNA test.

Subjects will receive Compound #10 200 mg/dose TID for 14 days.

The proposed trial will be performed in accordance with Good Clinical Practice (GCP) guidelines. Subjects will be monitored closely and results will be subject to review by regulatory authorities and an Institutional Review Board/Institutional Ethics Committee (IRB/IEC). Each subject must provide written informed consent prior to study drug administration.

Subjects must meet all of the following conditions to be eligible for enrollment into the study:

Males or females≧18 and ≦70 years of age.

Body mass index<30.

Capable of swallowing oral medication.

Chronic HCV infection, defined as a virological diagnosis of HCV for at months with persistent elevation of serum aminotransferase levels for at least 6 months.

Documentation of HCV genotype as determined by gene sequencing from an accredited laboratory. Patients with any HCV genotype may be enrolled.

Acute toxic effects as evaluated by Common Terminology Criteria for Adverse Events (CTCAE) Version 4.0 of any prior therapy resolved to Grade 1.

Confirmed screening laboratory values within the central laboratory ranges specified.

Exclusion Criteria:

Subjects meeting any of the following conditions will not be eligible for enrollment into the study:
1. History of drug-induced liver disease.
2. History of solid organ, bone marrow, or progenitor cell transplantation.
3. History of anti-HCV vaccination.
4. Treatment with PEG-IFN/ribavirin or any other HCV antiviral therapy within 4 weeks prior to start of study treatment.
5. Exposure to another investigational drug within 4 weeks prior to the start of study treatment.
6. History of major surgical procedure within 28 days prior to start of study treatment.
7. Donation of 1 unit (450 mL) whole blood in the last 8 weeks (56 days) of study treatment.
8. History of any malignancy except for nonmetastatic squamous or basal carcinoma of the skin.
9. Any of the following in the past 3 months: myocardial infarction, unstable angina, coronary/peripheral artery bypass graft, congestive heart failure (New York Heart Association Class III or IV), cerebrovascular accident, transient ischemic attack, other arterial thromboembolic event, or pulmonary embolism.
10. Severe liver disease (Child-Pugh score≧7).
11. Presence of encephalopathy or altered mental status of any etiology.
12. Known coagulopathy or bleeding diathesis.
13. Renal dysfunction requiring dialysis.
14. Evidence of ongoing systemic bacterial, fungal, or viral infection (including upper respiratory tract infections). Note: Subjects with localized cutaneous infections (including tinea versicolor), or with ungual fungal infections are eligible. Because Compound #10 will be used in patients with HIV infection, such patients with stable HIV infection and receiving highly active antiretroviral therapy will not be excluded.
15. Resting systolic blood pressure>180 mmHg or diastolic blood pressure>110 mmHg.
16. Pregnant or breast-feeding.
17. Concurrent participation in another therapeutic treatment trial.
18. Drug toxicology screen positive for any prohibited drugs, illicit substances, or alcohol.
19. Use of any drug that may alter Compound #10 metabolism due to the effects on cytochrome P450 enzyme 2C19 (including but not limited to carbamazepine, norethindrone, prednisone, rifampin, cimetidine, felbamate, fluoxetine, fluvoxamine, indomethacin, ketoconazole, lansoprazole, omeprazole, paroxetine, probenicid, ticlopidine, and topiramate). Use is precluded during the 14 days before the start of the study drug administration and during the treatment and follow-up period (ie, until collection of the final PK sample on the study).
20. Use of any drug the metabolism of which may be altered by Compound #10 because that drug is a substrate for cytochrome P450 2D6 (including but not limited to codeine, hydrocodone, oxycodone, lidocaine, flecamide, metoclopramide, chlorphenamine, chlorpheniramine, hydroxyzine, daunorubicin, doxorubicin, epirubicin, idarubicin, vincristine, aripiprazole, benztropine, clozapine, donepezil, haloperidol, mirtazapine, olanzapine, perphenazine, risperidone, thioridazine, zuclopenthixol, dextromethorphan, amphetamine, tamoxifen, duloxetine, fluoxetine, fluvoxamine, paroxetine, amitriptyline, clomipramine, desipramine, doxepin, imipramine, nortriptyline, carvedilol, metoprolol, penbutolol, propranolol, and timolol). Use is precluded during the 14 days prior to the start of study treatment and during the treatment and follow-up period (ie, until collection of the final PK sample on the study).
21. Anticipated need for alcohol and tobacco during the treatment and the follow-up period (ie, until collection of the final PK sample on the study).
22. Prior or ongoing clinically significant illness, medical condition, medical history, physical findings, ECG findings, or laboratory abnormality that, in the investigator's opinion, could affect the safety of the subject, or alter the absorption, distribution, metabolism, or excretion of the study drugs, or could impair the assessment of study results.

Study Drug Description:

Compound #10 will be provided in capsules for oral administration. The drug substance and product are manufactured and formulated following cGMP. Each capsule contains 20 mg of the active drug substance provided in hard gelatin, size 00 capsules. Compound #10 capsules will be provided in bulk bottles of 100 units for dispensing by the research unit pharmacist or designee. Compound #10 capsules should be stored at room temperature (˜15 to 30° C.).

Study Drug Administration:

In this study, treatment will comprise TID administration of Compound #10 for 14 days. Ideally, Compound #10 doses should be taken at ˜8-hour intervals (eg, at ˜7:00 AM, at ˜3:00 PM, and at ˜11:00 PM). If convenient for the patient, the drug may be taken during or within ˜30 minutes after a meal; however, administration with food is not required.

Safety Monitoring:

Subjects will be monitored closely for adverse events or laboratory abnormalities during the course of the study. For adverse events or laboratory abnormalities, the investigator should use his/her judgment in determining whether the event or abnormality is clinically significant, whether diagnostic evaluation is warranted, and whether potential interruption of study drug therapy is appropriate. In general, life-threatening (Grade 4) or severe (Grade 3) adverse events or laboratory abnormalities should be considered clinically significant, although recurrent or persistent moderate events (Grade 2) may also be considered clinically significant in certain circumstances. Reference should be made to the CTCAE, Version 4.0 (refer to http://ctep.cancer.gov/protocoldevelopment/electronic applications/docs/ctcaev4.pdf) for grading the severity of adverse events and laboratory abnormalities.

Pharmacokinetic Sampling:

Blood for trough Compound #10 concentrations will be collected at Days 2, 4, 7, 9 and 11, prior to the Compound #10 morning dose. The laboratory manual should be consulted for collection, processing, and shipping details.

Blood for Compound #10 concentrations over a 24-hour period will be collected from Day 1 to Day 2 and from Day 13 to Day 14. Blood should be collected immediately pre-dose and at approximately 1, 2, 3, and 4 hours after administration of the morning (˜7:00 AM) dose; immediately pre-dose and at approximately 1, 2, 3, and 4 hours after administration of the afternoon (˜3:00 PM) dose; and immediately pre-dose and at approximately 1, 2, 3, 4, and 8 hours after administration of the night (˜11:00 PM) dose (continuing into the next day). This means that blood will be collected at 0, 1, 2, 3, 4, 8, 9, 10, 11, 12, 16, 17, 18, 19, 20, and 24 hours relative to the morning Compound #10 dose on Days 1 and 13.

Samples will be stored at the bioanalytical lab for analysis of Compound #10 parent drug and metabolite(s) using a validated liquid chromatography with tandem mass spectrometry (LC-MS/MS) method. Thereafter, samples will be retained at the bioanalytical lab for potential follow-up analyses of Compound #10 and Compound #10 metabolites.

Blood Collection Summary:

The maximum amount of blood to be drawn on a single occasion is 41 mL and the total amount of blood to be drawn over the entire Screening and 28-day study period is 290 mL. These quantities of blood are within limits of 5 mL/kg of body weight for a single blood draw and 9.5 mL/kg of body weight for any 8 week period [National Institutes of Health 2009].

Statistical Considerations:

All subjects who receive≧1 dose of Compound #10 will be included in the analyses of safety and compliance. For anti-HCV activity, pharmacodynamic and PK parameters, evaluable populations of subjects will comprise all subjects who have sufficient baseline and on-study measurements to provide interpretable results for the test of interest.

Subject characteristics at study entry will be summarized with frequency tables for categorical variables, and with descriptive statistics such as the mean, standard deviation, median, and range, as appropriate, for quantitative variables.

Anti-HCV activity will be summarized will be characterized with appropriate descriptive statistics and graphical methods. Paired t-tests will be used to evaluate changes of HCV viral load over time relative to baseline. Number of patients with HCV viral load reduction by ≧1 log₁₀ will be summarized. Paired t-tests will also be used to evaluate changes of serum aminotransaminase values over time relative to baseline.

For each subject, the duration of treatment will be described. Dose modifications as implemented by the investigator will be listed. Reasons for such deviations from planned therapy will be listed and summarized. Compliance based on the subject daily diary will be described in terms of the proportion of drug actually taken relative to the amount that was prescribed (taking into account physician-prescribed reductions and interruptions). The type and timing of use of prior and concomitant medications will be listed and summarized.

Frequencies of adverse events will be tabulated by MedDRA System Organ Class, Preferred Term, worst severity, timing, outcome of the event, relationship to study drug, and seriousness.

Cytokine values will be characterized with appropriate descriptive statistics and graphical methods. Paired t-tests will be used to evaluate changes over time relative to baseline.

PK parameters for Compound #10 will be calculated using noncompartmental methods and summarized by visit.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed is:

1. A method for treating a viral infection in a human in need thereof, comprising administering to the human an effective amount of a compound having Formula (I): 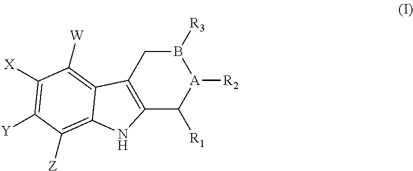
or a pharmaceutically acceptable salt, racemate, tautomer or stereoisomer thereof, wherein,
W is hydrogen; or halogen;
X is hydrogen; optionally substituted C₁ to C₈ alkyl; hydroxyl; halogen; thioether; sulfinyl; sulfonyl; cyano; or optionally substituted C₁ to C₈;
Y is hydrogen; optionally substituted C₁ to C₈ alkyl; or halogen;
Z is hydrogen; or C₁ to C₈ alkyl;
A is CH or N;
B is CH or N, with the proviso that at least one of A or B is N, and that when A is N, B is CH;
R₁ is hydroxyl; optionally substituted C₁ to C₈ alkyl; C₂ to C₈ alkenyl; C₂ to C₈ alkynyl; optionally substituted heterocyclyl; optionally substituted heteroaryl; or optionally substituted aryl;
R₂ is hydrogen; hydroxyl; halogen; optionally substituted heteroaryl; optionally substituted C₁ to C₈ alkyl; —C(O)—R_{c}; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; optionally substituted amino; optionally substituted aryl; —C(O)—Rₙ; optionally substituted heterocyclyl; heteroaryl; thiazoleamino; optionally substituted C₁ to C₈ alkyl; cycloalkyl; or optionally substituted C₂ to C₈ alkenyl;
R_{d} is at each occurrence independently hydrogen; C₂ to C₈ alkenyl; C₂ to C₈ alkynyl; optionally substituted aryl; cycloalkyl; or optionally substituted C₁ to C₈ alkyl;
Rₑ is hydrogen; optionally substituted C₁ to C₈ alkyl; optionally substituted cycloalkyl; or optionally substituted aryl;
R_{f} is optionally substituted C₁ to C₈ alkyl;
Rₙ is hydroxyl; C₁ to C₈ alkoxy; amino; optionally substituted aryl;
R₃ is hydrogen; or —C(O)—R_{g}; and
R_{g} is hydroxyl; optionally substituted amino; optionally substituted heteroaryl; or optionally substituted heterocyclyl.

2. The method of claim 1, wherein:
W is hydrogen; or halogen;
X is hydrogen; C₁ to C₈ alkyl optionally substituted with one or more halogen substituents; hydroxyl; halogen; thioether; sulfinyl; sulfonyl; cyano; or C₁ to C₈ alkoxy optionally substituted with aryl or C₁ to C₈ alkyl;
Y is hydrogen; C₁ to C₈ alkyl optionally substituted with one or more halogen substituents; or halogen;
Z is hydrogen; or C₁ to C₈ alkyl;
A is CH or N;
B is CH or N, with the proviso that at least one of A or B is N, and that when A is N, B is CH;
R₁ is hydroxyl; C₁ to C₈ alkyl optionally substituted with thioether, heteroaryl, or optionally substituted aryl; C₂ to C₈ alkenyl; C₂ to C₈ alkynyl; heterocyclyl optionally substituted with one or more substituents independently selected from halogen, oxo, amino, alkylamino, acetamino, thiol, or thioether; heteroaryl optionally substituted with one or more substituents independently selected from halogen, oxo, amino, alkylamino, acetamino, thiol, C₁ to C₈ alkoxy or thioether; or aryl optionally substituted with one or more independently selected Rₒ substituents;
Rₒ is a halogen; cyano; nitro; sulfonyl optionally substituted with C₁ to C₈ alkyl or heterocyclyl; amino optionally substituted with C₁ to C₆ alkyl, —C(O)—R_{b}, —C(O)O—R_{b}, sulfonyl, alkylsulfonyl or optionally substituted heterocyclyl; —C(O)—NH—R_{b}; heterocyclyl; heteroaryl; C₁ to C₈ alkyl optionally substituted with one or more substituents independently selected from hydroxyl, halogen, optionally substituted amino or optionally substituted heterocyclyl; —C(O)—Rₙ; or —ORₐ;
Rₐ is hydrogen; C₂ to C₈ alkenyl; C₂ to C₈ alkynyl; —C(O)—Rₙ; —C(O)O—R_{b}; —C(O)—NH—R_{b}; aryl; heteroaryl optionally substituted with halogen, amino, optionally substituted C₁ to C₈ alkyl; heterocyclyl optionally substituted with hydroxyl, optionally substituted C₁ to C₈ alkyl; C₁ to C₈ alkyl optionally substituted with one or more substituents independently selected from hydroxyl, halogen, cyano, optionally substituted C₁ to C₈ alkoxy, optionally substituted amino, optionally substituted monoalkylamino, optionally substituted dialkylamino, optionally substituted acetamino, sulfonyl, thioether, optionally substituted sulfonamide, —C(O)—R_{b}, —C(O)O—R_{b}, —OR_{g}, aryl, optionally substituted heterocyclyl, or optionally substituted heteroaryl;
R_{b} is hydroxyl; amino; or monoalkylamino or dialkylamino, wherein alkyl is independently optionally substituted with one or more substituents independently selected from hydroxyl, amino, alkylamino, C₁ to C₈ alkoxy, optionally substituted heterocyclyl; C₁ to C₈ alkoxy; C₂ to C₈ alkenyl; C₂ to C₈ alkynyl; aryl optionally substituted with one or more substituents independently selected from halogen or C₁ to C₈ alkoxy; heteroaryl; heterocyclyl optionally substituted with one or more substituents independently selected from acetamino, —C(O)O—Rₙ, heterocyclyl, or optionally substituted C₁ to C₈ alkyl; or C₁ to C₈ alkyl optionally substituted with one or more substituents independently selected from C₁ to C₈ alkoxy, aryl, optionally substituted amino, —C(O)O—Rₙ, or optionally substituted heterocyclyl;
R₂ is hydrogen; hydroxyl; halogen; heteroaryl optionally substituted with hydroxyl, optionally substituted C₁ to C₈ alkyl, optionally substituted aryl, heteroaryl, —C(O)O—R_{d}, —C(O)—N(R_{d}R_{d}); C₁ to C₈ alkyl optionally substituted with hydroxyl, C₁ to C₈ alkoxy, heterocyclyl, heteroaryl, or aryl; —C(O)—R_{c}; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; amino optionally substituted with one or more substituents independently selected from C₁ to C₈ alkyl or aryl; aryl optionally substituted with one or more substituents independently selected from halogen, haloalkyl, hydroxyl, C₁ to C₈ alkoxy, or C₁ to C₈ alkyl; —C(O)—Rₙ; heterocyclyl optionally substituted with —C(O)—Rₙ; heteroaryl; thiazoleamino; C₁ to C₈ alkyl optionally substituted with one or more substituents independently selected from halogen, C₁ to C₈ alkoxy, phenyloxy, aryl, —C(O)—Rₙ, —O—C(O)—Rₙ, hydroxyl, or optionally substituted amino; cycloalkyl; or C₂ to C₈ alkenyl optionally substituted with aryl;
R_{d} is at each occurrence independently hydrogen; C₂ to C₈ alkenyl; C₂ to C₈ alkynyl; aryl optionally substituted with one or more substituents independently selected from halogen, nitro, C₁ to C₈ alkyl, —C(O)O—Rₑ, or —ORₑ; cycloalkyl; or C₁ to C₈ alkyl optionally substituted with one or more substituents independently selected from halogen, C₁ to C₈ alkyl, C₁ to C₈ alkoxy, cycloalkyl, phenyloxy, optionally substituted aryl, heteroaryl, —C(O)—Rₙ, —C(O)O—Rₙ, or hydroxyl;
Rₑ is hydrogen; C₁ to C₈ alkyl optionally substituted with one or more substituents independently selected from halogen, —C(O)—Rₙ or C₁ to C₈ alkoxy; cycloalkyl optionally substituted with oxo; or aryl optionally substituted with one or more substituents independently selected from halogen or C₁ to C₈ alkoxy;
R_{f} is C₁ to C₈ alkyl optionally substituted with one or more substituents independently selected from halogen, hydroxyl, optionally substituted C₁ to C₈ alkoxy, cyano, optionally substituted aryl, or —C(O)—Rₙ;
Rₙ is hydroxyl; C₁ to C₈ alkoxy; amino; or aryl optionally substituted with halogen or C₁ to C₈ alkyl;
R₃ is hydrogen; or —C(O)—R_{g}; and
R_{g} is hydroxyl; amino optionally substituted with cycloalkyl or heteroaryl; heteroaryl optionally substituted with amino; or heterocyclyl optionally substituted with —C(O)—Rₙ.

3. The method of claim 1, wherein:
W is hydrogen; or halogen;
X is hydrogen; C₁ to C₈ alkyl optionally substituted with one or more halogen substituents; hydroxyl; halogen; thioether; sulfinyl; sulfonyl; cyano; or C₁ to C₈ alkoxy optionally substituted with aryl or C₁ to C₈ alkyl;
Y is hydrogen; C₁ to C₈ alkyl optionally substituted with one or more halogen substituents; or halogen;
Z is hydrogen; or C₁ to C₈ alkyl;
A is CH or N;
B is CH or N, with the proviso that at least one of A or B is N, and that when A is N, B is CH;
R₁ is hydroxyl; C₁ to C₈ alkyl optionally substituted with thioether, heteroaryl, or aryl optionally substituted with one or more independently selected Rₒ substituents; C₂ to C₈ alkenyl; C₂ to C₈ alkynyl; heterocyclyl optionally substituted with one or more substituents independently selected from halogen, oxo, amino, alkylamino, acetamino, thiol, or thioether; heteroaryl optionally substituted with one or more substituents independently selected from halogen, oxo, amino, alkylamino, acetamino, thiol, C₁ to C₈ alkoxy or thioether; or aryl optionally substituted with one or more independently selected Rₒ substituents;
Rₒ is a halogen; cyano; nitro; sulfonyl optionally substituted with C₁ to C₈ alkyl or heterocyclyl; amino optionally substituted with C₁ to C₆ alkyl, —C(O)—R_{b}, —C(O)O—R_{b}, sulfonyl, alkylsulfonyl or heterocyclyl optionally substituted with —C(O)O—Rₙ; —C(O)—NH—R_{b}; heterocyclyl; heteroaryl; C₁ to C₈ alkyl optionally substituted with one or more substituents independently selected from hydroxyl, halogen, amino or heterocyclyl, wherein amino and heterocyclyl are optionally substituted with one or more C₁ to C₈ alkyl substituents optionally substituted with one or more substituents independently selected from C₁ to C₈ alkoxy, amino, alkylamino, or heterocyclyl; —C(O)—Rₙ; or —ORₐ;
Rₐ is hydrogen; C₂ to C₈ alkenyl; C₂ to C₈ alkynyl; —C(O)—Rₙ; —C(O)O—R_{b}; —C(O)—NH—R_{b}; aryl; heteroaryl optionally substituted with halogen, amino, C₁ to C₈ alkyl optionally substituted with heterocyclyl or alkylsulfonyl, wherein heterocyclyl is optionally substituted with C₁ to C₈ alkyl; heterocyclyl optionally substituted with hydroxyl, C₁ to C₈ alkyl optionally substituted with hydroxyl, heterocyclyl, aryl, heteroaryl, —C(O)O—R_{b}, sulfonyl, C₁ to C₈ alkyl-sulfonyl or silyl-C₁ to C₈ alkyl-sulfonyl; C₁ to C₈ alkyl optionally substituted with one or more substituents independently selected from hydroxyl, halogen, cyano, C₁ to C₈ alkoxy, amino, monoalkylamino, dialkylamino, acetamino, sulfonyl, thioether, sulfonamide, —C(O)—R_{b}, —C(O)O—R_{b}, —OR_{g}, aryl, heterocyclyl, or heteroaryl, wherein the amino is optionally substituted with cycloalkyl, the alkyl of the monoalkylamino or dialkylamino is independently optionally substituted with one or more substituents independently selected from hydroxyl, C₁ to C₈ alkoxy, imino, amino, heterocyclyl, dialkylamino or heteroaryl optionally substituted with C₁ to C₈ alkyl, wherein the heteroaryl is optionally substituted with one or more substituents independently selected from —C(O)—NH—R_{b}, amino, cyano or heterocyclyl optionally substituted with one or more acetate or hydroxyl substituents, wherein the acetamino is optionally substituted with C₁ to C₈ alkyl, C₁ to C₈ alkoxy optionally substituted with C₁ to C₈ alkoxy, cycloalkyl, heteroaryl, sulfonyl, or alkylsulfonyl, wherein the heterocyclyl is optionally substituted with imino, —C(O)—Rₙ, —C(O)O—Rₙ, oxo or C₁ to C₈ alkyl optionally substituted with hydroxyl, further wherein the C₁ to C₈ alkoxy is optionally substituted with heterocyclyl, further wherein the sulfonamide is optionally substituted with C₁ to C₈ alkyl or cycloalkyl, further wherein the amino is optionally substituted with alkoxycarbonyl, alkylsulfonyl, cycloalkylsulfonyl, imidazole, isothiazole, pyrazole, pyridine, pyrazine, pyrimidine, pyrrole, thiazole, isoxazole, triazine or sulfonyl substituted with C₁ to C₈ alkyl, wherein pyridine, isoxazole, and thiazole are each optionally substituted with C₁ to C₈ alkyl;
R_{b} is hydroxyl; amino; or monoalkylamino or dialkylamino, wherein alkyl is independently optionally substituted with one or more substituents independently selected from hydroxyl, amino, alkylamino, C₁ to C₈ alkoxy, heterocyclyl optionally substituted with one or more substituents independently selected from C₁ to C₈ alkyl, oxo, —C(O)O—Rₙ, or heteroaryl optionally substituted with C₁ to C₈ alkyl; C₁ to C₈ alkoxy; C₂ to C₈ alkenyl; C₂ to C₈ alkynyl; aryl optionally substituted with one or more substituents independently selected from halogen or C₁ to C₈ alkoxy; heteroaryl; heterocyclyl optionally substituted with one or more substituents independently selected from acetamino, —C(O)O—Rₙ, heterocyclyl, or C₁ to C₈ alkyl optionally substituted with hydroxyl, C₁ to C₈ alkoxy, amino, monoalkylamino or dialkylamino; or C₁ to C₈ alkyl optionally substituted with one or more substituents independently selected from C₁ to C₈ alkoxy, aryl, amino, —C(O)O—Rₙ, or heterocyclyl, wherein the amino and heterocyclyl are optionally substituted with one or more substituents independently selected from C₁ to C₈ alkyl, oxo, or —C(O)O—Rₙ;
R₂ is hydrogen; hydroxyl; halogen; heteroaryl optionally substituted with hydroxyl, C₁ to C₈ alkyl optionally substituted with halogen, aryl or —C(O)—R_{c}, aryl optionally substituted with halogen or C₁ to C₈ alkoxy, heteroaryl, —C(O)O—R_{d}, —C(O)—N(R_{d}R_{d}); C₁ to C₈ alkyl optionally substituted with hydroxyl, C₁ to C₈ alkoxy, heterocyclyl, heteroaryl, or aryl; —C(O)—R_{c}; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
Rₑ is hydrogen; amino optionally substituted with one or more substituents independently selected from C₁ to C₈ alkyl or aryl; aryl optionally substituted with one or more substituents independently selected from halogen, haloalkyl, hydroxyl, C₁ to C₈ alkoxy, or C₁ to C₈ alkyl; —C(O)—Rₙ; heterocyclyl optionally substituted with —C(O)—Rₙ; heteroaryl; thiazoleamino; C₁ to C₈ alkyl optionally substituted with one or more substituents independently selected from halogen, C₁ to C₈ alkoxy, phenyloxy, aryl, —C(O)—Rₙ, —O—C(O)—Rₙ, hydroxyl, or amino optionally substituted with —C(O)O—Rₙ or —C(O)—Rₙ; cycloalkyl; or C₂ to C₈ alkenyl optionally substituted with aryl;
R_{d} is at each occurrence independently hydrogen; C₂ to C₈ alkenyl; C₂ to C₈ alkynyl; aryl optionally substituted with one or more substituents independently selected from halogen, nitro, C₁ to C₈ alkyl, —C(O)O—Rₑ, or —ORₑ; cycloalkyl; or C₁ to C₈ alkyl optionally substituted with one or more substituents independently selected from halogen, C₁ to C₈ alkyl, C₁ to C₈ alkoxy, cycloalkyl, phenyloxy, aryl, heteroaryl, —C(O)—Rₙ, —C(O)O—Rₙ, or hydroxyl, wherein the aryl is optionally substituted with one or more substituents independently selected from C₁ to C₈ alkyl, C₁ to C₈ alkoxy, cyano, halogen or haloalkyl;
Rₑ is hydrogen; C₁ to C₈ alkyl optionally substituted with one or more substituents independently selected from halogen, —C(O)—Rₙ or C₁ to C₈ alkoxy; cycloalkyl optionally substituted with oxo; or aryl optionally substituted with one or more substituents independently selected from halogen or C₁ to C₈ alkoxy;
R_{f} is C₁ to C₈ alkyl optionally substituted with one or more substituents independently selected from halogen, hydroxyl, C₁ to C₈ alkoxy, cyano, aryl, or —C(O)—Rₙ, wherein the C₁ to C₈ alkoxy is optionally substituted with one or more C₁ to C₈ alkoxy substituents and the aryl is optionally substituted with one or more substituents independently selected from halogen, hydroxyl, C₁ to C₈ alkoxy, cyano, or C₁ to C₈ alkyl;
Rₙ is hydroxyl; C₁ to C₈ alkoxy; amino; or aryl optionally substituted with halogen or C₁ to C₈ alkyl;
R₃ is hydrogen; or —C(O)—R_{g}; and
R_{g} is hydroxyl; amino optionally substituted with cycloalkyl or heteroaryl; heteroaryl optionally substituted with amino; or heterocyclyl, wherein the heterocyclyl is optionally substituted with —C(O)—Rₙ.

4. The method of claim 1, wherein the compound has the Formula (III): 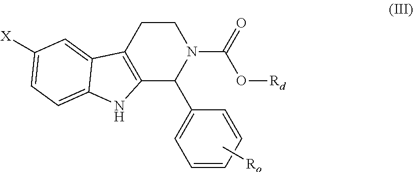
or a pharmaceutically acceptable salt, racemate, tautomer or stereoisomer thereof, wherein,
X is hydrogen; C₁ to C₈ alkyl optionally substituted with one or more halogen substituents; hydroxyl; halogen; thioether; sulfinyl, sulfonyl, cyano, or C₁ to C₈ alkoxy optionally substituted with aryl or C₁ to C₈ alkyl;
Rₒ is a halogen; cyano; nitro; sulfonyl optionally substituted with C₁ to C₈ alkyl or heterocyclyl; amino optionally substituted with C₁ to C₆ alkyl, —C(O)—R_{b}, —C(O)O—R_{b}, sulfonyl, alkylsulfonyl or heterocyclyl optionally substituted with —C(O)O—Rₙ; —C(O)—NH—R_{b}; heterocyclyl; heteroaryl; C₁ to C₈ alkyl optionally substituted with one or more substituents independently selected from hydroxyl, halogen, amino or heterocyclyl, wherein amino and heterocyclyl are optionally substituted with one or more C₁ to C₈ alkyl substituents optionally substituted with one or more substituents independently selected from C₁ to C₈ alkoxy, amino, alkylamino, or heterocyclyl; —C(O)—Rₙ; or —ORₐ;
Rₐ is hydrogen; C₂ to C₈ alkenyl; C₂ to C₈ alkynyl; —C(O)—Rₙ; —C(O)O—R_{b}; —C(O)—NH—R_{b}; aryl; heteroaryl optionally substituted with halogen, amino, C₁ to C₈ alkyl optionally substituted with heterocyclyl or alkylsulfonyl, wherein heterocyclyl is optionally substituted with C₁ to C₈ alkyl; heterocyclyl optionally substituted with hydroxyl, C₁ to C₈ alkyl optionally substituted with hydroxyl, heterocyclyl, aryl, heteroaryl, —C(O)O—R_{b}, sulfonyl, C₁ to C₈ alkyl-sulfonyl or silyl-C₁ to C₈ alkyl-sulfonyl; C₁ to C₈ alkyl optionally substituted with one or more substituents independently selected from hydroxyl, halogen, cyano, C₁ to C₈ alkoxy, amino, monoalkylamino, dialkylamino, acetamino, sulfonyl, thioether, sulfonamide, —C(O)—R_{b}, —C(O)O—R_{b}, —OR_{g}, aryl, heterocyclyl, or heteroaryl, wherein the amino is optionally substituted with cycloalkyl, the alkyl of the monoalkylamino or dialkylamino is independently optionally substituted with one or more substituents independently selected from hydroxyl, C₁ to C₈ alkoxy, imino, amino, heterocyclyl, dialkylamino or heteroaryl optionally substituted with C₁ to C₈ alkyl, wherein the heteroaryl is optionally substituted with one or more substituents independently selected from —C(O)—NH—R_{b}, amino, cyano or heterocyclyl optionally substituted with one or more acetate or hydroxyl substituents, wherein the acetamino is optionally substituted with C₁ to C₈ alkyl, C₁ to C₈ alkoxy optionally substituted with C₁ to C₈ alkoxy, cycloalkyl, heteroaryl, sulfonyl, or alkylsulfonyl, wherein the heterocyclyl is optionally substituted with imino, —C(O)—Rₙ, —C(O)O—Rₙ, oxo or C₁ to C₈ alkyl optionally substituted with hydroxyl, further wherein the C₁ to C₈ alkoxy is optionally substituted with heterocyclyl, further wherein the sulfonamide is optionally substituted with C₁ to C₈ alkyl or cycloalkyl, further wherein the amino is optionally substituted with alkoxycarbonyl, alkylsulfonyl, cycloalkylsulfonyl, imidazole, isothiazole, pyrazole, pyridine, pyrazine, pyrimidine, pyrrole, thiazole, isoxazole, triazine or sulfonyl substituted with C₁ to C₈ alkyl, wherein pyridine, isoxazole, and thiazole are each optionally substituted with C₁ to C₈ alkyl;
R_{b} is hydroxyl; amino; or monoalkylamino or dialkylamino, wherein alkyl is independently optionally substituted with one or more substituents independently selected from hydroxyl, amino, alkylamino, C₁ to C₈ alkoxy, heterocyclyl optionally substituted with one or more substituents independently selected from C₁ to C₈ alkyl, oxo, —C(O)O—Rₙ, or heteroaryl optionally substituted with C₁ to C₈ alkyl; C₁ to C₈ alkoxy; C₂ to C₈ alkenyl; C₂ to C₈ alkynyl; aryl optionally substituted with one or more substituents independently selected from halogen or C₁ to C₈ alkoxy; heteroaryl; heterocyclyl optionally substituted with one or more substituents independently selected from acetamino, —C(O)O—Rₙ, heterocyclyl, or C₁ to C₈ alkyl optionally substituted with hydroxyl, C₁ to C₈ alkoxy, amino, monoalkylamino or dialkylamino; or C₁ to C₈ alkyl optionally substituted with one or more substituents independently selected from C₁ to C₈ alkoxy, aryl, amino, —C(O)O—Rₙ, or heterocyclyl, wherein the amino and heterocyclyl are optionally substituted with one or more substituents independently selected from C₁ to C₈ alkyl, oxo, or —C(O)O—Rₙ;
Rₙ is hydroxyl; C₁ to C₈ alkoxy; amino; or aryl optionally substituted with halogen or C₁ to C₈ alkyl; and
R_{g} is hydroxyl; amino optionally substituted with cycloalkyl or heteroaryl; heteroaryl optionally substituted with amino; or heterocyclyl, wherein the heterocyclyl is optionally substituted with —C(O)—Rₙ.

5. The method of claim 1, wherein the compound has the Formula (IV): 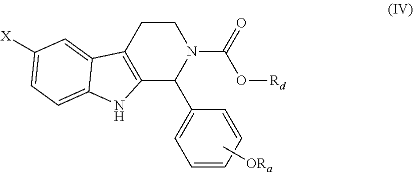
or a pharmaceutically acceptable salt, racemate, tautomer or stereoisomer thereof, wherein,
X is halogen;
Rₐ is H, C₁ to C₈ alkyl optionally substituted with one or more substituents independently selected from hydroxyl and halogen; and
R_{d} is phenyl substituted with one or more halogen substituents.

6. The method of claim 1, wherein the compound has the Formula (V): 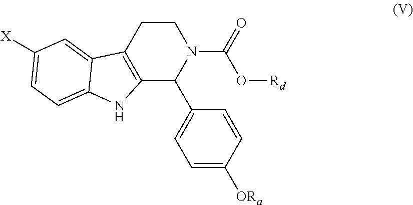
or a pharmaceutically acceptable salt, racemate, tautomer or stereoisomer thereof, wherein,
X is halogen;
Rₐ is H, C₁ to C₈ alkyl optionally substituted with one or more substituents independently selected from hydroxyl and halogen; and
R_{d} is phenyl substituted with one or more halogen substituents.

7. The method of claim 1, wherein the compound has the Formula (VI): 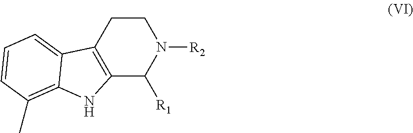
or a pharmaceutically acceptable salt, racemate, tautomer or stereoisomer thereof, wherein,
R₁ is hydroxyl; C₁ to C₈ alkyl optionally substituted with thioether, heteroaryl, or aryl optionally substituted with one or more independently selected Rₒ substituents; C₂ to C₈ alkenyl; C₂ to C₈ alkynyl; heterocyclyl optionally substituted with one or more substituents independently selected from halogen, oxo, amino, alkylamino, acetamino, thiol, or thioether; heteroaryl optionally substituted with one or more substituents independently selected from halogen, oxo, amino, alkylamino, acetamino, thiol, C₁ to C₈ alkoxy or thioether; or aryl optionally substituted with one or more independently selected Rₒ substituents;
R₂ is hydrogen; hydroxyl; halogen; heteroaryl optionally substituted with hydroxyl, C₁ to C₈ alkyl optionally substituted with halogen, aryl or —C(O)—R_{c}, aryl optionally substituted with halogen or C₁ to C₈ alkoxy, heteroaryl, —C(O)O—R_{d}, —C(O)—N(R_{d}R_{d}); C₁ to C₈ alkyl optionally substituted with hydroxyl, C₁ to C₈ alkoxy, heterocyclyl, heteroaryl, or aryl; —C(O)—R_{c}; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; amino optionally substituted with one or more substituents independently selected from C₁ to C₈ alkyl or aryl; aryl optionally substituted with one or more substituents independently selected from halogen, haloalkyl, hydroxyl, C₁ to C₈ alkoxy, or C₁ to C₈ alkyl; —C(O)—Rₙ; heterocyclyl optionally substituted with —C(O)—Rₙ; heteroaryl; thiazoleamino; C₁ to C₈ alkyl optionally substituted with one or more substituents independently selected from halogen, C₁ to C₈ alkoxy, phenyloxy, aryl, —C(O)—Rₙ, —O—C(O)—Rₙ, hydroxyl, or amino optionally substituted with —C(O)O—Rₙ or —C(O)—Rₙ; cycloalkyl; or C₂ to C₈ alkenyl optionally substituted with aryl;
R_{d} is at each occurrence independently hydrogen; C₂ to C₈ alkenyl; C₂ to C₈ alkynyl; aryl optionally substituted with one or more substituents independently selected from halogen, nitro, C₁ to C₈ alkyl, —C(O)O—Rₑ, or —ORₑ; cycloalkyl; or C₁ to C₈ alkyl optionally substituted with one or more substituents independently selected from halogen, C₁ to C₈ alkyl, C₁ to C₈ alkoxy, cycloalkyl, phenyloxy, aryl, heteroaryl, —C(O)—Rₙ, —C(O)O—Rₙ, or hydroxyl, wherein the aryl is optionally substituted with one or more substituents independently selected from C₁ to C₈ alkyl, C₁ to C₈ alkoxy, cyano, halogen or haloalkyl;
Rₑ is hydrogen; C₁ to C₈ alkyl optionally substituted with one or more substituents independently selected from halogen, —C(O)—Rₙ or C₁ to C₈ alkoxy; cycloalkyl optionally substituted with oxo; or aryl optionally substituted with one or more substituents independently selected from halogen or C₁ to C₈ alkoxy;
R_{f} is C₁ to C₈ alkyl optionally substituted with one or more substituents independently selected from halogen, hydroxyl, C₁ to C₈ alkoxy, cyano, aryl, or —C(O)—Rₙ, wherein the C₁ to C₈ alkoxy is optionally substituted with one or more C₁ to C₈ alkoxy substituents and the aryl is optionally substituted with one or more substituents independently selected from halogen, hydroxyl, C₁ to C₈ alkoxy, cyano, or C₁ to C₈ alkyl; and
Rₙ is hydroxyl; C₁ to C₈ alkoxy; amino; or aryl optionally substituted with halogen or C₁ to C₈ alkyl.

8. The method of claim 1, wherein the compound has the Formula (VII): 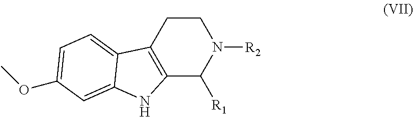
or a pharmaceutically acceptable salt, racemate, tautomer or stereoisomer thereof, wherein,
R₁ is hydroxyl; C₁ to C₈ alkyl optionally substituted with thioether, heteroaryl, or aryl optionally substituted with one or more independently selected Rₒ substituents; C₂ to C₈ alkenyl; C₂ to C₈ alkynyl; heterocyclyl optionally substituted with one or more substituents independently selected from halogen, oxo, amino, alkylamino, acetamino, thiol, or thioether; heteroaryl optionally substituted with one or more substituents independently selected from halogen, oxo, amino, alkylamino, acetamino, thiol, C₁ to C₈ alkoxy or thioether; or aryl optionally substituted with one or more independently selected Rₒ substituents;
R₂ is hydrogen; hydroxyl; halogen; heteroaryl optionally substituted with hydroxyl, C₁ to C₈ alkyl optionally substituted with halogen, aryl or —C(O)—R_{c}, aryl optionally substituted with halogen or C₁ to C₈ alkoxy, heteroaryl, —C(O)O—R_{d}, —C(O)—N(R_{d}R_{d}); C₁ to C₈ alkyl optionally substituted with hydroxyl, C₁ to C₈ alkoxy, heterocyclyl, heteroaryl, or aryl; —C(O)—R_{c}; —C(O)O—R_{d}; —C(O)C(O)—NH—R_{d}; —C(O)C(O)—O—R_{d}, —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; amino optionally substituted with one or more substituents independently selected from C₁ to C₈ alkyl or aryl; aryl optionally substituted with one or more substituents independently selected from halogen, haloalkyl, hydroxyl, C₁ to C₈ alkoxy, or C₁ to C₈ alkyl; —C(O)—Rₙ; heterocyclyl optionally substituted with —C(O)—Rₙ; heteroaryl; thiazoleamino; C₁ to C₈ alkyl optionally substituted with one or more substituents independently selected from halogen, C₁ to C₈ alkoxy, phenyloxy, aryl, —C(O)—Rₙ, —O—C(O)—Rₙ, hydroxyl, or amino optionally substituted with —C(O)O—Rₙ or —C(O)—Rₙ; cycloalkyl; or C₂ to C₈ alkenyl optionally substituted with aryl;
R_{d} is at each occurrence independently hydrogen; C₂ to C₈ alkenyl; C₂ to C₈ alkynyl; aryl optionally substituted with one or more substituents independently selected from halogen, nitro, C₁ to C₈ alkyl, —C(O)O—Rₑ, or —ORₑ; cycloalkyl; or C₁ to C₈ alkyl optionally substituted with one or more substituents independently selected from halogen, C₁ to C₈ alkyl, C₁ to C₈ alkoxy, cycloalkyl, phenyloxy, aryl, heteroaryl, —C(O)—Rₙ, —C(O)O—Rₙ, or hydroxyl, wherein the aryl is optionally substituted with one or more substituents independently selected from C₁ to C₈ alkyl, C₁ to C₈ alkoxy, cyano, halogen or haloalkyl;
Rₑ is hydrogen; C₁ to C₈ alkyl optionally substituted with one or more substituents independently selected from halogen, —C(O)—Rₙ or C₁ to C₈ alkoxy; cycloalkyl optionally substituted with oxo; or aryl optionally substituted with one or more substituents independently selected from halogen or C₁ to C₈ alkoxy;
R_{f} is C₁ to C₈ alkyl optionally substituted with one or more substituents independently selected from halogen, hydroxyl, C₁ to C₈ alkoxy, cyano, aryl, or —C(O)—Rₙ, wherein the C₁ to C₈ alkoxy is optionally substituted with one or more C₁ to C₈ alkoxy substituents and the aryl is optionally substituted with one or more substituents independently selected from halogen, hydroxyl, C₁ to C₈ alkoxy, cyano, or C₁ to C₈ alkyl; and
Rₙ is hydroxyl; C₁ to C₈ alkoxy; amino; or aryl optionally substituted with halogen or C₁ to C₈ alkyl

9. The method of claim 1, wherein the viral infection is selected from Bunyaviridae, Coronaviridae, Filoviridae, Flaviviridae, Paramyxoviridae, Picornaviridae, Orthomyxoviridae, Rhabdoviridae, Hepadnaviridae, Reoviridae, Retroviridae, Adenoviridae, Herpesviridae, Papillomaviridae or Papovaviridae.

10. The method of claim 9, wherein the Flaviviridae virus is selected from West Nile virus, hepatitis C virus, yellow fever virus and dengue virus; the Paramyxoviridae virus is selected from parainfluenza virus and respiratory syncytial virus; the Picornaviridae virus is selected from poliovirus, hepatitis A virus, Coxsackievirus and rhinovirus; the Coronaviridae virus is selected from severe acute respiratory syndrome coronavirus; the Orthomyxoviridae virus is selected from influenza virus; the Filoviridae virus is selected from Ebola and Marburg viruses; the Retroviridae virus is selected from human immunodeficiency virus and human T cell leukemia virus; the Hepadnaviridae virus is hepatitis B virus; or the virus is selected from herpes simplex virus, Kaposi's sarcoma-associated herpesvirus, adenovirus, vaccinia virus or human papilloma virus.

11. The method of claim 10, wherein the virus is West Nile virus, hepatitis C virus, dengue virus, respiratory syncytial virus, poliovirus, severe acute respiratory syndrome coronavirus, influenza virus, parainfluenza virus, human immunodeficiency virus, human T cell leukemia viruses, herpes simplex virus or vaccinia virus.

12. The method of claim 11, wherein the virus is West Nile virus, hepatitis C virus, dengue virus, respiratory syncytial virus, poliovirus, influenza virus, parainfluenza virus or human immunodeficiency virus.

13. The method of claim 12, wherein the hepatitis C virus is the hepatitis C virus genotype 1a, the hepatitis C virus genotype 1b or the hepatitis C virus genotype 2a.

14. The method of claim 1, further comprising the administration of a second active agent.

15. The method of claim 14, wherein the second active agent is a HCV protease inhibitor, a NS3 protease inhibitor, a NS4a protease cofactor inhibitor; a HCV NS5b polymerase inhibitor; a NS4b inhibitor, NS5a inhibitor, a IRES inhibitor, a p7 inhibitor, an entry inhibitor, a fusion inhibitor, a helicase inhibitor, ribavirin, a ribavirin analogue, ribavirin and a nonpegylated interferon or a pegylated interferon, a TLR agonist, a cyclophilin inhibitor, a caspase inhibitor, a pancaspase inhibitor, an immunomodulator, an antiinflammatory, a broad spectrum immune stimulator, an antifibrotic, an antioxidant, a hemopurifier, a IMPDH inhibitor, a glycosidase inhibitor, a glucosidase inhibitor, a HCV therapeutic vaccine, a A3 adenosine receptor agonist, a polypeptide eglin c analog inhibitor, a human pancreatic secretory trypsin and minibody repertoire inhibitor or a monoclonal antibody and fragment thereof; a HIV inhibitor, a HBV inhibitor, a RNA inhibitor, a RNAi, an anti-phospholipid therapy, a protein therapeutic, an interferon replacement agent, or a botanical or non-specific pharmaceutical.

16. The method of claim 1, wherein the effective amount of the compound is in a range of from about 0.001 mg per kg per day to about 1500 mg per kg per day.

17. A method for inhibiting or reducing viral replication or the production of viral RNA or DNA, viral protein or virus induced cytopathic effects, comprising contacting an effective amount of a compound having Formula (I) with a cell or a cell line that produces viral RNA or DNA, viral protein or a virus induced cytopathic effect or is induced to produce viral RNA or DNA, viral protein or a virus induced cytopathic effects: 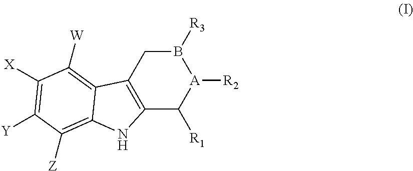
or a pharmaceutically acceptable salt, racemate, tautomer or stereoisomer thereof, wherein,
W is hydrogen; or halogen;
X is hydrogen; optionally substituted C₁ to C₈ alkyl; hydroxyl; halogen; thioether; sulfinyl; sulfonyl; cyano; or optionally substituted C₁ to C₈;
Y is hydrogen; optionally substituted C₁ to C₈ alkyl; or halogen;
Z is hydrogen; or C₁ to C₈ alkyl;
A is CH or N;
B is CH or N, with the proviso that at least one of A or B is N, and that when A is N, B is CH;
R₁ is hydroxyl; optionally substituted C₁ to C₈ alkyl; C₂ to C₈ alkenyl; C₂ to C₈ alkynyl; optionally substituted heterocyclyl; optionally substituted heteroaryl; or optionally substituted aryl;
R₂ is hydrogen; hydroxyl; halogen; optionally substituted heteroaryl; optionally substituted C₁ to C₈ alkyl; —C(O)—R_{c}; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; optionally substituted amino; optionally substituted aryl; —C(O)—Rₙ; optionally substituted heterocyclyl; heteroaryl; thiazoleamino; optionally substituted C₁ to C₈ alkyl; cycloalkyl; or optionally substituted C₂ to C₈ alkenyl;
R_{d} is at each occurrence independently hydrogen; C₂ to C₈ alkenyl; C₂ to C₈ alkynyl; optionally substituted aryl; cycloalkyl; or optionally substituted C₁ to C₈ alkyl;
Rₑ is hydrogen; optionally substituted C₁ to C₈ alkyl; optionally substituted cycloalkyl; or optionally substituted aryl;
R_{f} is optionally substituted C₁ to C₈ alkyl;
Rₙ is hydroxyl; C₁ to C₈ alkoxy; amino; optionally substituted aryl;
R₃ is hydrogen; or —C(O)—R_{g}; and
R_{g} is hydroxyl; optionally substituted amino; optionally substituted heteroaryl; or optionally substituted heterocyclyl.

18. A method for inhibiting or reducing the viral replication or the production of viral RNA or DNA, viral protein or virus induced cytopathic effects in a subject in need thereof, comprising administering to the subject an effective amount of a compound having Formula (I): 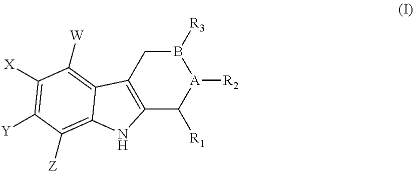
or a pharmaceutically acceptable salt, racemate, tautomer or stereoisomer thereof, wherein,
W is hydrogen; or halogen;
X is hydrogen; optionally substituted C₁ to C₈ alkyl; hydroxyl; halogen; thioether; sulfinyl; sulfonyl; cyano; or optionally substituted C₁ to C₈;
Y is hydrogen; optionally substituted C₁ to C₈ alkyl; or halogen;
Z is hydrogen; or C₁ to C₈ alkyl;
A is CH or N;
B is CH or N, with the proviso that at least one of A or B is N, and that when A is N, B is CH;
R₁ is hydroxyl; optionally substituted C₁ to C₈ alkyl; C₂ to C₈ alkenyl; C₂ to C₈ alkynyl; optionally substituted heterocyclyl; optionally substituted heteroaryl; or optionally substituted aryl;
R₂ is hydrogen; hydroxyl; halogen; optionally substituted heteroaryl; optionally substituted C₁ to C₈ alkyl; —C(O)—Rₑ; —C(O)O—R_{d}; —C(O)C(O)—NH—R_{d}; —C(O)C(O)—O—R_{d}; —C(O)—N(R_{d}R_{d}); —C(S)—N(R_{d}R_{d}); —C(S)—O—Rₑ; —S(O₂)—Rₑ; —C(NRₑ)—S—Rₑ; or —C(S)—S—R_{f};
R_{c} is hydrogen; optionally substituted amino; optionally substituted aryl; —C(O)—Rₙ; optionally substituted heterocyclyl; heteroaryl; thiazoleamino; optionally substituted C₁ to C₈ alkyl; cycloalkyl; or optionally substituted C₂ to C₈ alkenyl;
R_{d} is at each occurrence independently hydrogen; C₂ to C₈ alkenyl; C₂ to C₈ alkynyl; optionally substituted aryl; cycloalkyl; or optionally substituted C₁ to C₈ alkyl;
Rₑ is hydrogen; optionally substituted C₁ to C₈ alkyl; optionally substituted cycloalkyl; or optionally substituted aryl;
R_{f} is optionally substituted C₁ to C₈ alkyl;
Rₙ is hydroxyl; C₁ to C₈ alkoxy; amino; optionally substituted aryl;
R₃ is hydrogen; or —C(O)—R_{g}; and
R_{g} is hydroxyl; optionally substituted amino; optionally substituted heteroaryl; or optionally substituted heterocyclyl.
